SEQUENCE LISTING (17 sequences)

SEQ ID NO: 1 - protein, 624 aa, Homo Sapien
LSQRFPKYLYEIARYLYEIARFQNALLVRFQNALLVRQTALVELVKKYLYEIARLDELRDEGKYLYEIARRKQTALVELVKCCTESLVNRHPDYSVVLLLRAVMDDFAAFVEKAVMDDFAAFVEKAAFTECCQAADKYICENQDSISSKRHPDYSVVLLLRVPQVSTPTLVEVSRLKECCEKPLLEKCCAAADPHECYAKCCAAADPHECYAKADDKETCFAEEGQKKVPQVSTPTLVEVSRKVPQVSTPTLVEVSRAEFAEVSKLVTDLTKQNCELFEQLGEYKYICENQDSISSKLKQEPERNECFLQHKEQLKAVMDDFAAFVEKRPCFSALEVDETYVPKRPCFSALEVDETYVPKNECFLQHKDDNPNLPRVFDEFKPLVEEPQNLIKVFDEFKPLVEEPQNLIKAAFTECCQAADKAACLLPKEFNAETFTFHADICTLSEKEFNAETFTFHADICTLSEKERVHTECCHGDLLECADDRADLAKVHTECCHGDLLECADDRADLAKQNCELFEQLGEYKFQNALLVRLVRPEVDVMCTAFHDNEETFLKLVRPEVDVMCTAFHDNEETFLKLVRPEVDVMCTAFHDNEETFLKKLVRPEVDVMCTAFHDNEETFLKK

SEQ ID NO: 2 - protein, 193 aa, Homo Sapien
DYAEVGRGSFPVIQAKGSFPWCAKVGYVSGVIGRVGYVSGWGRVTSEQDWVQKDIAPILTLYVGKSCAVAEYGVYVKYVNILPVADQDQCRTMLPVADQDQCIRVMPICIPSKENADIGRVMPICIPSKDYABIGRSPVGVNLNEHTFCAGMSKSPVGVQPILNEHTFCAGMSKSPVGVQPLNEHTFCAGMSK

SEQ ID NO: 3 - protein, 622 aa, Homo Sapien
FQFLALLVRFQNALLVRQTALVELVKKYLYEIARLDELRDEGKCCTESLVNRLVIEVTEFAKHPDYSVVLLLRAVMDDFAAFVEKAVMDDFVFVEKFQNALLVRYTKAVMDDFIAFVEKAAFTECCQAADKYICENQDSISSIRHPDYSVVLLLRRHPDYSVVLLLRVPVSTPTLVEVSRLIECCEKPLLEICCAAADPHECYAKCCAAADPHECYAKDVFLGMFLYEYARADDKETCFAEEGQKKVPQVSTPTLVEVSRKVPQVSTPTLVEVSRAEFAEVSKLVTDLTKNCELFEQLGEYKYICEIQDSISSKLKHPYFYAPELLFFAKHPYFYAPELLFFAKRRHPYFYAPELLFFAIRPCFSALEVDETYVPKRPCFSLEVDETYVPKVPVSRPTLVEVSRNLGKVFDEFIPLVEEPQNLIIVFDEFKPLVEEPILIKVHTECCHGDLLECADDREFNAETFNHADICRLSEKEFNAETFTFHADICTLSEKERVHTECCHGDLLECADDRADLAKVHTECCHGDLLECADDRADLAKNCELFEQLGEYKFQNALLVRLVRPEVDVMCTIFHDNEELHKLVRPEVDVMCTAFHDNEETFLKKLVRPEVDVMCTIFHDNE

SEQ ID NO: 4 - protein, 71 aa, Homo Sapien
EGETKAVKLLELTGPKLLELRGPKSGLSTGWTQLSKSGLSTGWTLSKHQFLLTGDTQGRHQFLLTGDLTGR

SEQ ID NO: 5 - protein, 69 aa, Homo Sapien
NYYKLRNYYKLRGSFPWQAKLRTEGDGVYTLNNEKAVGDKLPECEAVCGKPKAVGDKLPECEAVCGKPK

SEQ ID NO: 6 - protein, 277 aa, Homo Sapien
DTLMISRALPFPIEKALPAPIEKDTLMSRNQVSLTCLVKNQVSLTCLVKGPSVFPLAPSSKEPQVYTLPPSREPQVYTLPPSRQTIPDYRRMIGQGAFIWYVDGVEVHIAKFTNVYVDGVEVHTAKEPQVYTLPPSRDELTKEPQYTLPPSRDELTKTPEVTCVVVDVSHEDPETVKTPEVTCVVVDVSHEDPEVKTPEVTCVVVDVSHEDPEVKGFYPSDIAVEWESNGQPENNYKWQQGIVFSCSIRVHEALHNHYTQKWQQGNVFSCSVMHEAL

SEQ ID NO: 7 - protein, 494 aa, Homo Sapien
RPCFSALEVDETYVPKRPCFSALEVDETYVPKNECFLQHKDDNPNLPRVFDEFKPLVEEPQNLIKVFDEFKPLVEEPQNLIKAFTECCQADKAACLLPKEFNAETFTFHADCTLSEKEFNAETFNHIDICRLSEKERVHRECCHGDLLECADDRADLAKVHTECCHGDLLECADDRADLAKQNCELFEQLGEYKFQNALLVRLVRPEVDVMCTAFHDNEETFLILVRPEVDVMCRAFHDNEETFLKLVRPEVDVMCTAFHDNEETFLIKLVRPEVDVMCRAFHDNEETFLKINQVSLTCLVKNQVSLTCLVKEPQVYTLFPSREPQVYTLPPSREPQYTLPPSRDELTKEPQVYTLPPSRDELTKTTPPVLDSDGSFFLYSKGFYPSDIAVEWESIGQPEIIYKGFYPSDIAVEWESIGQPENNYKWQGIVFSCSVMHEALHNHYTQKWQGNVFSCSVILHEALHNHYTQKWQQGRVFSCSVMHEALHRHYTQK

SEQ ID NO: 8 - protein, 899 aa, Homo Sapien
RPDPWAKDSAHGFLKSCHTFVGRAPNHAVITRASYLDCIRYLGEEYVKKSCHTAVGRKASYLDCIRHQTVPQTTGGKWCALSHHERDSGFQMNQLRDSGFQIVLNQLRSASDLTWDNLKSASDLTWDILKHSTIFENLANKEFQLFSSPHGKEGYYGYTGAFREGYRGYTGAFRWCAVSEHEATKKDSGFQMNQLRKDSGFQMIQLRDYELLCLDGTRKSASDLWVDNLKSVIPSDGPSVACVKMYLGYEYVTAIRSKEFQLFSSPHGKSKEFQLFSSPHGKMYLGYEVTAIRLKCDEWSVNSVGKCSTSSLLEACTFRCSTSSLLEACTFRDQYELLCLDITRDLLFRDDTVCLKDLLFRDDTVCLAKFDEFFSEGCAPGSKKPVEEYANCHLARKPVEEYANGHLARTAGWNIPMGLLYNKHSTIFENLNKADREDPTFYYAVAVVKKCSTSSLLEACTFRDCHLAQVPSHTVVARFDEFFSEGCAPGSIKLCMGSGLNLCEPNNIIEGVSAETTEDGIAKEGTCPEAPTDECKPVKADRDQYELLCLDITRADRDQYELLCLDTTRNLNEKDYELLCLDGTRKPVDEYIDCHLAQVPSHTVVARRPCFSALEVDETYVPKRPCFSALEVDETYVPKNECFLQHKDDNPNLPRVFDEFKPLVEEPQNLIKVFDEFKPLVEEPQNLIKAFTECCQADKAACLLPKEFNAETFTFHADCTLSEKEFNAETFNHIDICRLSEKERVHRECCHGDLLECADDRADLAKVHTECCHGDLLECADDRADLAKQNCELFEQLGEYKFQNALLVRLVRPEVDVMCTAFHDNEETFLILVRPEVDVMCRAFHDNEETFLKLVRPEVDVMCTAFHDNEETFLIKLVRPEVDVMCRAFHDNEETFLKI

SEQ ID NO: 9 - protein, 210 aa, Homo Sapien
ATVVYQGERATVVYQGEREHSSLAFWKEHSSLAFWKKATVVYQGERVCPFAGILENGAVRVCPFAGILENGAVRTCPKPDDLPFSTVVPLKTCPKPDDLPFSTVVPLKCSYTEDAQCIDGTIEVPKTFYEPGEEITYSGKPGYVSRTFYEPGEEITYSCKPGYVSRATFGCHDGYSLDGPEEIECTKGPFPSRPDNGFVNYPAKPTLYYK

SEQ ID NO: 10 - protein, 165 aa, Homo Sapien
ATVVYQGERATVIYQGEREHSSLAFWKEHSSLAFWKKATVVYQGERVCPFAGILENGAVRICPFAGILENGAVRTCPKPDDLPFSTVIPLKTCPKPDDLPFSTIVPLKCSYTEDAQCIDGTIEVPKTFYEPGEEITYSCKPGYSRTFYEPGEEITYSGKPGYVSR

SEQ ID NO: 11 - protein, 143 aa, Homo Sapien
GVGHPYRRYGIDWASGRYGIDWASGRYVSEAHKQDGSVDFFRQDGSVDFFRTFAHYATFRTFAHYATFRGEPGDPVNLLRRQDGSVDFFRRQDGSVDFFRLLGEVDHYQLALGKLLGEVDHYQLALGKMGPKGEPGDPVNLLR

SEQ ID NO: 12 - protein, 772 aa, Homo Sapein
TMMSVSRTMMSVSRNASKVANKGAASRFVKNALWEPKLDPLEQAKLDPLEQAKFVKNALWEPKEINFYKQKQQSAKQKQQSAKRTMFQKRTMFQKDEEILLKHHLEFGKMKVQEEKAFLNCDRMSLFMYRFHIPQEALVREPLDESGWMIKHHLEFGKTLLRHHLEFGKTLLREINFYKVIDYILHGKVKQMFRDSMLGDRMMSTRRVENTAKRRVENTAKVKQMFRVSGPGVRRSQKPGRRRTASAPTKVKQALGLKFLQVEQKSKSNPNLRLSHSLGLPGGTRRRLSHSLGLPGGTRYLMAGISDEDSLARTSSSTQLKSEGSGGTQLKMQKSEGSGGTQLKMQKSEGSGGTQLKTINPFGEQSRVPSAFAAIYSKTSSSTQLKSEGSGGTQLKMQKSEGSGGTQLKMQKSEGSGGTQLKTINPFGEQSRVPSAFAAIYSKTSSSTQLKSEGSGGTQLKMQKSEGSGGTQLKMQKSEGSGGTQLKTINPVHSDDEVFERVKQMFRDSMLGDRMMSTRRVENTAKRRVENTAKVKQMFRVSGPGVRRSQKPGRRRTASAPTKVKQALGLKFLQVEQKSKSNPNLRLSHSLGLPGGTRRRLSHSLGLPGGTRYLMAGISDEDSLARTIYLCRSSADSSRKKFASALSKVWTSQLKLPYEQWKDLRVWTSQLKYQKQQKSGRSPAPRVWFQNRMPSPRPRLTENQVRAQLAARLKAQLAARLKELDRERELDRERMGGTTSTRMGGTTSTRGGTTSTRRSEEERAKQAKELDR

SEQ ID NO: 13 - protein, 2360 aa, Homo Sapien
KSSMVALKYKLLESKNLEVEHRLRQQAEIKLRQQAEIKESSLSRQSKNYEALKQRQKGQLEDLEKQKGQLEDLEKETEVLQTDHKQASEYESLISKDLEDRYNQLLKDLEDRYNQLLKSVSGKTPGDFYDRSSSQENLLDEVMKTLVTLREDLVSEKLIEVERNNATLQAEKKTEDTYFISSAGKPTPGTQGKTLLEQNMESKDLFHVEQRTLLEQNMESKDLFHVEQRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRAAQIAGAVRATPAHRARGNMRSCRGNMRSCRLTDFGFGRSCRVLLHMRSCRVLLHMRGHQGGGPAASAPGLRGAPGHPLRPQEVRCENVLLSPDERRLEAGWFQPFLQPRALGQGGARLMALGPIRLMALGPIRFVTISGQKDFTELQKKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRQLNLLQKRFSNIDKFALTAETKRNFSNMKSLTGYLEKSLTGYLEKAELEHWKIPAWWKKRLELWLRRLELWLRVNDLIEFRSVAMMVPDRSVAMMVPDRTWREMDIRNMKLIESKPKNMKLIESKPKAVLLIGEQGTAKLLSDIFIPALRLLSDIFIPALRVDTLHYAWEKTSIIDFTVTMKLKEASESVAALSKRLLSDIFIPALRLPNPAYTPEISARLVSVLSTIINSTKKNFSNMKMMLAEYQRERWTEQSQEFAAQTKELLSHFNHQNMDALLKVTREILNNHGKDAAPGASKLRDAAPGASKLRGVVDHLLLRGVVDHLLLRQPWHGKAMQRNNEFPVFDEFNNEFPVFDEFGGSPAVTLLISEKASEAGATAPKASARISAPNEFDVMFKLSRDDISTAAGMVKDDVDMLRDYDALRKLEQEYSRRENGQLLRRENGQLLRAHGPEVQAHNKAHGPEVQAHNKKAANEEMEALRLADVEQELSFKKLADVEQELSFKKDDNSATKTLSAAARLQTEVELAESKLKAANEEMEALRQIKVQKGSEPLGISIVSGEKDLNPGVKKDLNPGVKKMSLGQLQSARGTCSGFEPHSWRGVACLGCKGTCSGFEPH

SEQ ID NO: 14 - protein, 2811 aa, Homo Sapien
ESEDQKRMGGTTSTRRKSSMVALKYKLLESKNLEVEHRLRQQAEIKLRQQAEIKESSLSRQSKNYEALKQRQKGQLEDLEKQKGQLEDLEKETEVLQTDHKQASEYESLISKDLEDRYNQLLKDLEDRYNQLLKSVSGKTPGDFYDRSSSQENLLDEVMKTLVTLREDLVSEKLIEVERNNATLQAEKKTEDTYFISSAGKPTPGTQGKTLLEQNMESKDLFHVEQRTLLEQNMESKDLFHVEQRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRAAQIAGAVRATPAHRARGNMRSCRGNMRSCRLTDFGFGRSCRVLLHMRSCRVLLHMRGHQGGGPAASAPGLRGAPGHPLRPQEVRCENVLLSPDERRLEAGWFQPFLQPRALGQGGARLMALGPIRLMALGPIRFVTISGQKDFTELQKKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRLMALGPIRLMALGPIRFVTISGQKDFTELQKYSEIQDRYSEIQDRKEVMEHRQQVQFMLKQQVQFMLKLEEEVEACKVVKAQIQEQKNCPISAKLERNCPISAKLERAFSIDIIRHKQISEQLNALNKQISEQLNALNKLLEVWIEFGREKTLLPEDSQKGDLRFVTISGQKQPVYDTTIRTGRQPVYDTTIRTGRARQEQLELTLGRTGSLEEMTQRLRNTISVKAVCDYRLNDALDRLEELKEETYNQLLDKGRLMLLSRDDSGSGSKQTTGEEVLLIQEKLLEVWIEFGRIKELNPEEGEMVEEKHMLEEEGTLDLLGLKKLLPQAEMFEHLSGKQEFIDGILASKFPTTKALIAEHQTFMEEMTRLLSDTVASDPGVLQEQLATTKMSELRVTLDPVQLESSLLREALAGLLVTYPNSQEAENWKEALAGLLVTYPNSQEAENWKEMFSQLADLDDELDGMGAIGRQLNLLQKRFSNIDKFALTAETKRNFSNMKSLTGYLEKSLTGYLEKAELEHWKIPAWWKKRLELWLRRLELWLRVNDLIEFRSVAMMVPDRSVAMMVPDRTWREMDIRNMKLIESKPKNMKLIESKPKAVLLIGEQGTAKLLSDIFIPALRLLSDIFIPALRVDTLHYAWEKTSIIDFTVTMKLKEASESVAALSKRLLSDIFIPALRLPNPAYTPEISARLVSVLSTIINSTKKNFSNMKMMLAEYQRERWTEQSQEFAAQTKELLSHFNHQNMDALLKVTREAALTLPREAALTLPREILNNHGKDAAPGASKLRDAAPGASKLRGVVDHLLLRGVVDHLLLRQPWHGKAMQRNNEFPVFDEFNNEFPVFDEFGGSPAVTLLISEKASEAGATAPKASARISAPNEFDVMFKLSRDDISTAAGMVKDDVDMLRDYDALRKLEQEYSRRENGQLLRRENGQLLRAHGPEVQAHNKAHGPEVQAHNKKAANEEMEALRLADVEQELSFKKLADVEQELSFKKDDNSATKTLSAAARLQTEVELAESKLKAANEEMEALRQIKVQKGSEPLGISIVSGEKDLNPGVKKDLNPGVKKMSLGQLQSARGTCSGFEPHSWRGVACLGCKGTCSGFEPHRQAVQDLRADVVNKALREKMELEMRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRSWCQELEKRGEHGRLLSLWRVNVELQLQGDSAQGQKLMALGPIR

SEQ ID NO: 15 - protein, 2374 aa, Homo Sapien
RQAVQDLRADVVNKALREKMELEMRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRLKNEEVESERSWCQELEKRIQVLEDQRTRLSALNEALALDKGEHGRLLSLWRQDYITRSAQTSRQDYITRSAQTSRQDLAEQLQGLSSAKRVQALEEVLGDLRELTTQRQLMQERERLQEMLMGLEAKHNVQLRSTLEQVERVNVELQLQGDSAQGQKRQAVQDLRADVVNKALRSWCQELEKRGEHGRLLSLWRVNVELQLQGDSAQGQKLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKSEQLMALGPIRDFTELQKYSEIQDRYSEIQDRKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKLMALGPIRDFTELQKKEVMEHRQQVQFMLKQQVQFMLKNHWEELSKKVVKAQIQEQKNCPISAKLERNCPISAKLERQQLEETSEIRLRLEEYEQRQISEQLNALNKQISEQLNALNKLLEVWIEFGRGDLRFVTISGQKALLELVPWRARQPVYDTTIRTGRQPVYDTTIRTGRARQEQLELTLGRTGSLEEMTQRLRNTISVKAVCDYRNTISVKAVCDYRLNDALDRLEELKEETYNQLLDKGRLMLLSRDDSGSGSKQTTGEEVLLIQEKLLEVWIEFGRIKELNPEEGEMVEEKEGLDKLVSDANEQYKHMLEEEGTLDLLGLKKLLPQAEMFEHLSGKALIAEHQTFMEEMTRKALIAEHQTFMEEMTRKIGPQLKELNPEEGEMVEEKLLSDTVASDPGVLQEQLATTKMSELRVTLDPVQLESSLLREIQDKLDQMVFFWEDIKMSELRVTLDPVQLESSLLRNGQALLKQTTGEEVLLIQEKTNFPYVRSDILGHLRSDILGHLRNIFKRPLGSRNIFKRPLGSRQSGVVPFIFQAKSGFPLLKEFVQRTLSGTPEVHSNKRTLSGTPEVHSNKRAAEGIPKLLVLITGGKYPPPGEMGASEVLLGAFSIKMKPLDGSALYTGSALDFVRSAGSRIEDGVLQFLVLLVAGRVDGPASNLKQSGVVPFIFQAKGNRVSPTMKDSLLELSPVERDSLLELSPVERYPGMFIALSKNGKYPGMFIALSKNGKRLYCNVGIGFHLQALPDGRRLYCNVGIGFHLQALPDGRMAGASPAVPHERARMAGASPAVPHERARLFEDMVTKLQALRALGVGGAGCGVQGLASLARTTGLSDQQVVCDLDHRAVEALLQAVRMNGEYRGRGGGNFSGKWRTSEQTPQFLLSTKELTEGGEVTNLIPDIATELRTLACMGLAIHQVLTKDLERDAPEKTLACMGLAIHQVL

SEQ ID NO: 16 - protein, 1858 aa, Homo Sapien
RQAVQDLRRQAVQDLREKMELEMRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLKNEEVESERIQVLEDQRTRLSALNEALALDKLHMEKADVVNKHFLEMKSATDRQDYITRSAQTSRQDYITRSAQTSRQDLAEQLQGLSSAKELTTQRQLMQERERLQEMLMGLEAKERVSLLETLLQTQKHNVQLRSTLEQVERVNVELQLQGDSAQGQKLSGSLLTCCLRLTVGAQSRRQAVQDLRRQAVQDLRLHMEKADVVNKHFLEMKSATDRLQSSQLQSCRVLKVNVELQLQGDSAQGQKLSGSLLTCCLRLTVGAQEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKSEQAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKSVAYMPYAEVKSVAYMPYAEVKSVAYMPYAEVKRSSTSPCGTSKSPNRSSTSPCGTSKSPNRRALEQEAQMHNTAARYSVPPVLQPAPHQVITNLPEWHVVSSKLMALGPIRYSEIQDRYSEIQDRQQVQFMLKQQVQFMLKVVKAQIQEQKNCPISAKLERQQLEETSEIRQISEQLNALNKQISEQLNALNKLLEVWIEFGRGDLRFVTISGQKQPVYDTTIRTGRQPVYDTTIRTGRARQEQLELTLGRTGSLEEMTQRLRNTISVKAVCDYRNTISVKAVCDYRLNDALDRLEELKFEQLCLQQQEKEETYNQLLDKGRLMLLSRDDSGSGSKQTTGEEVLLIQEKLLEVWIEFGRIKLLEVWIEFGRIKELNPEEGEMVEEKEIKFLDVLELAEKNDECVLEDNSQRTKEGLDKLVSDANEQYKHMLEEEGTLDLLGLKKLLPQAEMFEHLSGKLDQMVFFWEDIKARALIAEHQTFMEEMTRKALIAEHQTFMEEMTRKDENYYQLEELAFRVMRLLSDTVASDPGVLQEQLATTKMSELRVTLDPVQLESSLLRSTVMVRVGGGWMALDEFLVKFSQQYSTIVKDYELQLMTYKHLFCPDLLRHLFCPDLLRDKVAFITGGGSGIGFRIAEIFMRAEFFADVVPAVRDILFPYIEENVKMKAEFFADVVPAVRQLQGHMWRAAFTAGRLLFGHSTEGDILELVDGHFDTKIGHKVYIYSSGSVEAQKLLFGHSTEGDILELVDGHFDTKKLPASEGVGKKLPASEGVGKNVILMAHDGKFYMVSYYERFYMVSYYERWQDRCLEVRGPITAVAFAPDGRSLAALKNMAHHKGLQECFPAICRYSHNSLMVQAIKYSHNSLMVQAIKLPASCLPASDSFRVIIWTENGQSYIYKHTCKALTFLLLQPPSPKEHLLDDEEEDEEIMRQRYDQRYLISGGVDFSVIIWQGWSQLAAMHCVMLP

SEQ ID NO: 17 - protein, 2581 aa, Homo Sapien
KLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKKLPASEGVGKFYMVSYYERWQDRCLEVRWQDRCLEVRGPITAVAFAPDGRSLAALKNMAHHKSLAALKNMAHHKGLQECFPAICRYSHNSLMVQAIKYSHNSLMVQAIKLPASCLPASDSFRVIIWTENGQSYIYKEHLLDDEEEDEEIMRHTCKALTFLLLQPPSPKHALSLIATARPPAFITTIAKEHLLDDEEEDEEIMRQREHLLDDEEEDEEIMRQRCQTIHGHKGPITAVAFAPDGRYDQRYLISGGVDFSVIIWDIFSGEMKEVQLVESGAEVKSDDTAVYYCARSDDTAVYYCARASGYTFTGYYMHWVRVTMTRDTTISTAYMELSRIAAAGDAFDIWGQGTMVTVSSLMALGPIRLMALGPIRYSEIQDRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKWADHEVRAMESTATAAVAAELVSADKMAMESTATAAVAAELVSADKAMESTATAAVAAELVSADKMSGRLMALGPIRLMALGPIRYSEIQDRQQVQFMLKQQVQFMLKVVKAQIQEQKNCPISAKLERNCPISAKLERMPPLIPAEVDKQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKLMALGPIRLMALGPIRQQVQFMLKQQVQFMLKVVKAQIQEQKNCPISAKLERNCPISAKLERMPPLIPAEVDKQQLEETSEIRQISEQLNALNKLLEVWIEFGRLLEVWIEFGREKTLLPEDSQKGDLRFVTISGQKGDLRFVTISGQKQPVYDTTIRTGRQPVYDTTIRTGRARQEQLELTLGRTGSLEEMTQRLRNTISVKAVCDYRFEQLCLQQQEKEETYNQLLDKGRLMLLSRDDSGSGSKLLEVWIEFGRIKELNPEEGEMVEEKEIKFLDVLELAEKHMLEEEGTLDLLGLKQEFIDGILASKFPTTKALIAEHQTFMEEMTRALIAEHQTFMEEMTRKALIAEHQTFMEEMTRKWLKETEGSIPPTETSMSAKLLSDTVASDPGVLQEQLATTKLLSDTVASDPGVLQEQLATTKEIQDKLDQMVFFWEDIKEALAGLLVTYPNSQEAENWKEMFSQLADLDDELDGMGAIGRFSQQYSTIVKDYELQLMTYKSYAEELAKISYSLFTALRIVMNMNKSTRDKLDQVSSEIKDKLDQVSSEIKMININILSVCKMININILSVCKGVFVQSVLPYFVATKLAKMININILSVCKMTQLVLPGMVERMININILSVCKMTQLVLPGMVERYEDEINKEDLARLLRGFSSGSAVVSGGSRRSTSSFSCLSRHGGGGGGFGGGGFGSRHGGGGGGFGGGGFGSRGSSSGGGYSSGSSSYGSGGRQCKNVQDAIADAEQRSISISVAGGGGGFGAAGGFGGRDYQELMNVKLALDVEIATYRNPGSLRGRLETHPCRGSIGQSAIPRSPCPIRSPLPARASAPWASLSTRADSGLRMSPLETNKGSIGQSAIPRATSASLPQETPFALSVVWAPRR

(19) United States
(12) Patent Application Publication
(10) Pub. No.: US 2017/0198027 A1
(43) Pub. Date: Jul. 13, 2017

(54) PROCESS OF AFOD AND AFCC AND MANUFACTURING AND PURIFICATION PROCESSES OF PROTEINS

(71) Applicant: Kieu Hoang, Agoura Hills, CA (US)

(72) Inventor: Kieu Hoang, Agoura Hills, CA (US)

(21) Appl. No.: 15/239,388

(22) Filed: Aug. 17, 2016

Related U.S. Application Data
(62) Division of application No. 13/756,478, filed on Jan. 31, 2013, now abandoned.
(60) Provisional application No. 61/593,183, filed on Jan. 31, 2012.
(60) Provisional application No. 61/593,196, filed on Jan. 31, 2012.
(60) Provisional application No. 61/648,281, filed on May 17, 2012.
(60) Provisional application No. 61/692,273, filed on Aug. 23, 2012.
(60) Provisional application No. 61/710,930, filed on Oct. 8, 2012.
(60) Provisional application No. 61/593,164, filed on Jan. 31, 2012.

(51) Int. Cl. C07K 14/76; C07K 16/00; C07K 14/47

(52) U.S. Cl. CPC C07K 14/76; C07K 14/47; C07K 14/4716; C07K 16/00

(57) ABSTRACT

Manufacturing and purification processes of proteins, KH 1-through KH-52, and more KH proteins are being discovered in good healthy cells—named KH CELLS. KH CELLS are good healthy cells in which the RNA synthesizes good proteins that: 1) Send signal to the damaged, sick, and bad cells that triggers that synthesis of good proteins that transform these cells to become GOOD healthy cells; 2) Send signal to the other currently undamaged cells to synthesis of good proteins to protect them from being damaged, infected and prone to DNA and other cellular alterations; and 3) Send signal to the body to produce new cells that are healthy and forbid them from being affected by intra- and extracellular damaging signals. The mechanism that governs these processes is that the KH good healthy cells provide innate good signals that make good proteins to boost the immune system.

### CROSS REFERENCE TO RELATED APPLICATIONS

This application is a divisional application of U.S. patent application Ser. No. 13/756,478, filed Jan. 31, 2013, which claims the benefit under 35 USC 120 of the filing dates of provisional application No. 61/593,164, filed on Jan. 31, 2012, provisional application No. 61/593,183, filed on Jan. 31, 2012, provisional application No. 61/593,196, filed on Jan. 31, 2012, provisional application No. 61/648,281, filed on May 17, 2012, provisional application No. 61/692,273, filed on Aug. 23, 2012 and provisional application No. 61/710,930, filed on Oct. 8, 2012, all of which are hereby incorporated herein by reference in their entireties.

Process of AFOD and AFCC and Manufacturing and Purification processes of existing discovered and newly discovered proteins, KH 1-through KH-52, and more KH proteins are being discovered in GOOD HEALTHY CELLs—named KH CELLS. KH CELLS are GOOD HEALTHY CELLS in which the RNA synthesizes good proteins that:

1—Send signal to the DAMAGED, SICK, AND BAD CELLS that triggers that synthesis of good proteins that transform these cells to become GOOD healthy cells.

2—Send signal to the other currently undamaged cells to synthesis of good proteins to protect them from being DAMAGED, INFECTED and PRONE to DNA and other cellular alterations.

3—Send signal to the body to produce new cells that are healthy and forbid them from being affected by intra- and extracellular damaging signals.

The mechanisms that govern these processes is the KH good healthy cells provide innate good signals that make good proteins to boost the immune system in order to CURE, TO PROTECT, and TO PREVENT diseases, viruses infections, bacteria infections, auto immune disease, neurological disorder, all type of solid and blood cancer, coagulation, diabetic, inhibitor, immune deficiency, muscle and nerve repair and restoration from Human, animal or substances by the method of fractionation, purification, recombinant DNA, monoclonal antibody, transgenic and expression of cells from the cultured GOOD HEALTHY CELLS.

INVENTOR: Kieu Hoang

30423 Canwood St. #120

Agoura Hills, Calif. 91301

### BRIEF DESCRIPTION OF THE DRAWINGS

FIG. 1. Process flow chart of the manufacturing of tile AFOD RAAS 101® from pool of the plasma to fraction V for further process into a human albumin containing ALB Uncharacterized protein, HPR 31 kDa protein, ALB Uncharacterized protein, A1BG isoform 1 of Alpha-1B-glycoprotein, HPR Haptoglobin and KH51.

FIG. 2. Protein analysis of RAAS human albumin against human album import from other manufacturers. RAAS Albumin containing 1—ALB Uncharacterized protein, 2—HPR 31 kDa protein, 3—ALB Uncharacterized protein, 4—A1BG isoform 1 of Alpha-1B-glycoprotein, 5-HPR Haptoglobin and 6-KH51 proteins

FIG. 2.1 Protein analysis of RAAS Human albumin containing the protein ACTC1 Actin, alpha cardiac muscle 1.

FIG. 3. Protein analysis of International import Company 1 human albumin containing only HPR31 kDa protein.

FIG. 4. Protein analysis of International import Company 2 human albumin containing only HPR31 kDa and Albumin Uncharacterized proteins.

FIG. 5. Protein analysis of International import Company 3 human albumin containing only HPR31 kDa, Albumin Uncharacterized and A1BG isoform 1 of Alpha-1B-glycoprotein proteins.

FIG. 6. Process flow chart of the manufacturing of the AFOD RAAS 102® from Fraction II+III paste.

FIG. 7. Protein analysis of Immunoglobulin from fraction II+III. Beside Immunoglobulin there are two other proteins 120/E19 IGHV4-31; IGHG1 44 kDa protein and 191/H18 IGHV4-31; IGHG1 32 kDa.

FIG. 7.1 Process analysis of Immunoglobulin containing the protein IGHV4-31;IGHG1 Putative uncharacterized protein DKFZp686G11190.

FIG. 8. Process flow chart of the manufacturing of tile AFOO RAAS 103® from fraction III paste

FIG. 9. Protein analysis of Immunoglobulin from fraction III containing 193/H20 TF serotransferrin, 194/H21 APOH beta2-glycoprotein 1, 195/H22 eDNA FLJ5165, moderately similar to beta-2-glycoprotein, 196/H23 FCN3 isoform 1 of Ficolin-3.

FIG. 10. Process flow chart of the manufacturing of the AFOO RAAS 104® HBig purification process from Fraction II+III paste.

FIG. 11. Protein analysis of HBIG beside the Immunoglobulin proteins, containing the protein TF serotransferrin.

FIG. 12. Protein analysis comparison between Immunoglobulin from II+III paste vice versa immunoglobulin produced from fraction III paste and Hepatitis B immunoglobulin produced from fraction II+III paste showing the different protein in each of the product bedsides the main Immunoglobulin protein analysis.

FIG. 13. Protein analysis for AFOO RAAS 102®, AFOO RAAS 103® and AFOD RAAS 104®

FIG. 14. Process flow chart for AFOD RAAS 105®

FIG. 14a. Process flow chart for AFOO RAAS 105® FIG. 15. Process flow chart for AFOD RAAS 106®

FIG. 16. Process flow chart for purification process of AFOO RAAS 107® (CP98) FIG. 17. 20 electropherosis of plasma derived protein CP 98 kDa

FIG. 18. Process flow chart for purification process of AFOO RAAS 108® (A1AT) FIG. 19. 20 electropherosis of plasma derived protein A1AT

FIG. 20. Process flow chart for purification process of AFOO RAAS 109® (Transferrin)

FIG. 21. 20 electropherosis of plasma derived protein Transferrin

FIG. 22. Process flow chart for purification process of AFOO RAAS 110® (AntiThrombin III)

FIG. 22a. Process flow chart for purification process of AFOO RAAS 110® (AntiThrombin III from fraction III)

FIG. 23. 20 electropherosis of plasma derived protein AntiThrombin III

FIG. 24. Process flow chart for purification process of AFOO RAAS 111® (Human Albumin from fraction IV)

FIG. 25. 20 electropherosis of plasma derived protein Human Albumin from fraction IV

FIG. 26. Process flow chart for purification process of AFOO RAAS 112® (Human Albumin from Fraction III)

FIG. 27—Photograph of Cryopaste and FVIII

FIG. 28. Process flow chart for purification process of AFCC RAAS 101® (Human Coagulation Factor VIII)

FIG. 29. 20 electropherosis of plasma derived protein Human coagulation Factor VIII FIG. 30. Process flow chart for purification process of AFCC RAAS 102® (Human Fibrinogen)

FIG. 31. 20 electropherosis of plasma derived protein Human Fibrinogen

FIG. 32. Process flow chart for purification process of AFCC 103® (High Concentrate Human Fibrinogen)

FIG. 33. 20 electropherosis of plasma derived protein High Concentrate Human Fibrinogen

FIG. 34. Process flow chart for purification process of AFCC RAAS 104® (Human Thrombin)

FIG. 35. 20 electropherosis of plasma derived protein Human Thrombin

FIG. 36. Process flow chart for purification process of AFCC RAAS 105® (Human Prothrombin Complex)

FIG. 37. 20 electropherosis of plasma derived protein Human Prothrombin

FIG. 38. Process flowchart of AFCC RAAS 106@ Purification process from Fr. IV1+IV4 paste

FIG. 38a. 20 electropherosis of AFCC from fraction IV.

FIG. 38b. 20 electropherosis of Anti Thrombin III.

FIG. 38c. 20 electropherosis of CP98.

FIG. 38d. 20 electropherosis of Transferrin.

FIG. 38e, 20 electropherosis of Alpha 1 Antitrypsin.

FIG. 38f 20 electropherosis of Human Albumin.

FIG. 39. Process flowchart for Recombinant Factor VIII

FIG. 40. Process flowchart for Monoclonal Antibodies.

FIG. 41. Process flowchart for manufacturing of AFOD RAAS and AFCC RAAS products by using the direct cell from cell culture for expression to synthesize the desired already discovered or newly found proteins.

FIGS. 42-1 through 42-6 show Dose-dependent curves (by GraphPad Prism) showing AFCC KH has 100%.

percentage of inhibition of HIV virus like the reference compound.

FIG. 43. All products have shown a low percentage of inhibition.

FIGS. 44-1 through 44-18. Log compound ug/mL showing inhibition of HCV in AFOD KH 70% and AFCC RAAS 1 50%, AFCC RAAS 4 40% to compare with Ribavirin which reach only 50%

FIGS. 451 through 45-18-. Log compound ug/mL showing inhibition of HCV in AFOD KH 70% and AFCC RAAS 1 50%, AFCC RAAS 4 40% to compare with Ribavirin which reach only 50%;

FIG. 46. CCK8 testing method. In vitro testing for Lung Cancer cells in RAAS current plasma derived products.

FIG. 47. CCK8 testing method. In vitro testing for Lung Cancer cells in RAAS new plasma derived products.

FIG. 47a. In vitro studies of the different proteins vs Lung Cancer at 0%, 2%) and 10% concentration of the product

FIG. 48. High concentration of rONA products with lung cancer cell.

FIG. 49. High concentration of rONA products with lung cancer cell

FIG. 50. Recombinant and monoclonal products in inhibiting lung cancer cell

FIG. 50a. In vitro studies of the different recombinant products vs Lung Cancer at 0%, 2% and 10% concentration of the product.

FIG. 50b. In vitro studies of the different recombinant products vs Lung Cancer at 0%, 2% and 10% concentration of the product.

FIG. 51. 5% samples from animal source with feta bovine serum, bovine albumin, bovine IVIG, pig thrombin and pig fibrinogen.

FIG. 52. 5% sample from animal source with feta bovine serum, bovine albumin, bovine IVIG, pig thrombin and pig fibrinogen with lung cancer cell.

FIG. 53. KH101 medium alone, KH101 medium consist of 50 g of paste of rice in 1 liter of water for injection.

FIG. 54. KH101 medium alone, KH101 medium consist of 50 g of paste of rice in 1 liter of water for injection with cell count analysis showing nearly 20 million cells.

FIG. 55. Product AFCC alone showing nearly 8,000 cells.

FIG. 56. Product AFCC mixed with KH101 medium.

FIG. 57. Product AFCC mixed with KH101 medium after 5 days in bioreactor, which has reach 4.5 million cell count

FIG. 58. APOA1 product alone with cell count with nearly 20,000 cells.

FIG. 59. APOA1 product with KH101 medium.

FIG. 60. APOA1 with KH101 medium after 5 days in bioreactor which after cell analysis has reached 4 million cell count.

FIG. 61. AFOD Product alone with cell count with nearly 10,000 cells.

FIG. 62. AFOD Product with KH101 medium

FIG. 63. AFOD product with KH101 medium after 5 days in bioreactor which after cell analysis has reached 4.6 million cell count.

FIG. 64. Factor VIII alone with cell count with nearly 5,400 cells.

FIG. 65. Factor VIII with KH 101 medium.

FIG. 66. Factor VIII with KH101 medium after 5 days in bioreactor which after cell analysis has reached 3.4 million cell count.

FIG. 67. Liver fatty change of Rabbit after treatment with AFOD RAAS 101.

FIG. 68. Comparison of fat deposit on heart from vehicle rabbit and AFOD RAAS 101 treated rabbit.

FIG. 69. Comparison of atherosclerosis in aorta from vehicle rabbit and treated rabbit

FIG. 70. Pictures of aorta from vehicle control rabbit.

FIG. 71. Pictures of aorta from rabbit treated with a low dose of AFOD RAAS 101.

FIG. 72. Pictures of aorta from rabbit treated with a medium dose of AFOD RAAS 101.

FIG. 73. Pictures of aorta from rabbit treated with a high dose of AFOD RAAS 101.

FIG. 74. Pictures of aorta from rabbit treated with a positive control (Lipitor)

FIG. 75. Analysis of body weight in 18 aPOe MICE.

FIG. 76. Blood plasma lipid profile at three time points in 18 Apo E(−/−) mice.

FIG. 77. Illustration of Aorta.

FIG. 78. Oil red staining procedure.

FIG. 79. image analysis and procedure of aorta.

FIG. 80. Aorta photos of vehicle, control and treated mice.

FIG. 81. Graph showing results of the sum area of atherosclerotic plaque. (mm2).

FIG. 81a. Area of atherosclerotic plaque on aorta.

FIG. 81b. Photos of treated and control aortas.

FIG. 81c. Results of the atherosclerotic plaque

FIG. 81d. Results of the mean density.

FIG. 81e. Results of the area percent

FIG. 82. Effect of APOA1 on body weight

FIG. 83. Effect of APOA1 on food intake.

FIG. 84. Comparison of the lipid profile of ApoE mice fed with common diet and high fat diet.

FIG. 85. Effect of RAAS antibody on total cholesterol.

FIG. 86. Net change of plasma total cholesterol after 8 weeks.

FIG. 87. Effect of RAAS antibody on triglyceride.

FIG. 88. Effect of RAAS antibody on High Density Lipoprotein.

FIG. 89. Effect of RAAS antibody on Low Density Lipoprotein.

FIG. 90. Effect of RAAS antibody on Atherosclerosis plaque lesion area.

FIG. 91. Effect of RAAS antibody on the percent of plaque area.

FIG. 92. Effect of RAAS antibody on the percent of plaque area after 2 weeks

FIG. 93. Analysis area of the aortic plaque.

FIG. 94. Analysis of tile root plaque area.

FIG. 95. Analysis of tile percent of the root plaque area.

FIG. 96. Analysis area of the artery.

FIG. 97. Analysis of plaque area from root to right renal area.

FIG. 98. Analysis of plaque area percentage from root to right renal area.

FIG. 99. The effect of the aortic inner lumen area

FIG. 100. The mean density of the effect of the aortic lumen area.

FIG. 101. The effect of RAAS antibody on liver weight.

FIG. 102. The effect of RAAS antibody on liver weight index.

FIG. 103. The effect of RAAS antibody on fasting overnight blood glucose

FIG. 104. Image of aorta red oil staining.

FIG. 105. Image of aorta red oil staining in different groups.

FIG. 106. Images of red stained aorta in negative control.

FIG. 107. Images of red stained aorta in vehicle control.

FIG. 108. Images of red stained aorta treated with APOA1 high dose.

FIG. 109. Images of red stained aorta treated with APOA1 medium dose.

FIG. 110. Images of red stained aorta treated with APOA1 low dose.

FIG. 111. Images of red stained aorta in positive control (Atorvastatin).

FIG. 112. Effect of AFOD on body weight.

FIG. 113. Effect of products on blood glucose (fasting 6 hrs)

FIG. 114. Effect of products o fasting overnight of blood glucose.

FIG. 115. The effect of AFOD on plasma insulin.

FIG. 116. The effect of AFOD on HOMA-IR

FIG. 117. The effect of AFOD, AFCC, APOA1 on body weight.

FIG. 118. The effect of AFOD, AFCC and APOA1 on fasted 6 hours of blood glucose.

FIG. 119. The effect of AFOD, AFCC and APOA1 on overnight fasted blood glucose.

FIG. 120. The effect of AFOD, AFCC and APOA1 on plasma insulin

FIG. 121. The effect of AFOD, AFCC and APOA1 on plasma HOMA-IR

FIG. 122. The effect of AFOD, AFCC and APOA1 on plasma lipid.

FIG. 123. The effect of AFOD, AFCC and APOA1 on liver weight.

FIG. 124. Plasma insulin level in db/db mice during two periods of study.

FIG. 125. Breast cancer 4T1-Iuc orthotopic model growth curve

FIG. 126. Breast cancer 4T1-Iuc orthotopic model growth curve for AFOD RAAS 1, 2, 3 and 4.

FIG. 127. Breast cancer 4T1-Iuc orthotopic model growth curve for AFOD RAAS 5 and 6.

FIG. 128. Breast cancer 4T1-Iuc orthotopic model growth curve for AFOD RAAS 1, 2, 3, 4, 5 and 6 and AFOD KH and AFCC KH

FIG. 129. Breast cancer 4T1-Iuc orthotopic model growth curve for AFOD RAAS 1, 2, 3 and 4.

FIG. 130. Breast cancer 4T1-Iuc orthotopic model growth curve for AFOD RAAS 5 and 6 and AFOD KH and AFCC KH.

FIG. 131. Breast cancer 4T1-Iuc orthotopic model body weight change for AFOD RAAS 1, 2, 3 and 4.

FIG. 132. Breast cancer 4T1-Iuc orthotopic model body weight change for AFOD RAAS 1, 2, 3 and 4.

FIG. 133. Breast cancer 4T1-Iuc orthotopic model body weight change for AFOD RAAS 5 and 6 and AFOD KH and AFCC KH.

FIG. 134. Fluorescence images of the whole body for vehicle, Gemcitabine, AFOD RAAS 1/8, AFOD RAAS2 and AFOD RAAS 3.

FIG. 135. Fluorescence images of the whole body for AFOD RAAS 4, AFOD RAAS 5, AFOD RAAS 6, AFOD KH and AFCC KH

FIG. 136. Anti-tumor efficacy of FS+AFOD in POX model U-00-0117

FIG. 137. Weights of tumors on day 24 after treatment

FIG. 138. Photograph of each tumor for each group.

FIG. 139. Relative change of body weight (%) of different groups

FIG. 140. Photo of nude mice with MDA-MB-231-Luc tumor cells.

FIG. 141. Photo of 10 nude mice group 2-5 which have been implanted with tumor cells from the 2-5 mice positive control group using Docetaxel in another study done at another CRO lab.

FIG. 142. Photo of nude mice with MDA-MB-231-Luc tumor cells transferred from 2-5 positive control group using Docetaxel

FIG. 143. Graph showing the tumor volume of Mice #6-10 from the study done from Jul. until Nov. 11, 2011 when the dead body of mouse #6-10 was removed from one CRO lab to another one for further study.

FIG. 144. Pictures of mouse #6-10 taken from Aug. 23, 2011 to Nov. 3, 2011 showing the growth of the tumor which had been detached from the body was under recovery from breast cancer using AFCC proteins for treatment.

FIG. 145. The tissue from the area of mouse #6-10 where the tumor had been detached was used to implant in the 10 nude mice 66 days after re-implantations show no tumor growth.

FIG. 146. Table showing tumor growth of mouse #6-10 after second re-implantation.

FIG. 147. Graph showing tumor growth after re-implantation of various mice including 6-10.

FIG. 148. Photo of nude mice group #6-10 with mice $5-1 and #5-5 showing growth of the tumor.

FIG. 149. Photo of mice 6-10 after re-implantation, showing tumor growth which has been inhibited by using AFCC KH proteins from Feb. 29, 2012.

FIG. 150. Graph of mouse #4-6 recovery within 24 days.

FIG. 151. Mouse #4-6 grew the tumor on August 23rd and self-detached from the body Sep. 1, 2011.

FIG. 152. Photo of mouse #4-6 completely recovered.

FIG. 153. Photo of 10 mice in group #4-6

FIG. 154. Photo of nude mice #4-6 with no tumor growth.

FIG. 155. Photo of nude mice used as negative control with no tumor.

FIG. 156. Photo of nude mice C57BU6 used as negative control with no tumor.

FIG. 157. The percent of B cells in peripheral blood.

FIG. 158. The percent of activated B lymphocytes in peripheral blood.

FIG. 159. The percent of monocytes/macrophages in peripheral blood.

FIG. 160. The percent of mDC and pDC in peripheral blood.

FIG. 161. The percent of CD3 T cells in spleen.

FIG. 162. The percent of B cells in spleen.

FIG. 163. The percent of mDC and pDC in spleen.

FIG. 164. The percent of activated B lymphocytes in spleen.

FIG. 165. The percent of monocytes/macrophages in spleen.

FIG. 166. The percent of granulocytes in spleen.

FIG. 167. The percent of CD3 T cells in draining lymph nodes.

FIG. 168. The percent of B cells in draining lymph nodes.

FIG. 169. The percent of mDC and pDC in draining lymph nodes.

FIG. 170. The percent of granulocytes in draining lymph nodes.

FIG. 171. The percent of monocytes and macrophages in draining lymph nodes.

FIG. 172. The percent of activated B lymphocytes in draining lymph nodes.

FIG. 173. Effect of AFOD RAAS2 on H1N1 caused mortality.

FIG. 174. The average body weight change in mice infected with H1N1 influenza.

FIGS. 175A-D. Effects of pretreatment of AFOD on the behavioral performance.

FIGS. 176A-D. Effects of pretreatment+post treatment of AFOD on the behavioral performance.

FIGS. 177A-B. TH staining of the SN. Rats were perfused and the brains were fixed for IHC study.

FIGS. 178A-B. Effects of daily injection of AFOD on adjusting step test.

FIG. 179. Effects of daily injection of AFOD on rotation

FIG. 180. TH staining of the SN.

FIG. 181. Body weight changes caused with AFCC treatment in mice.

FIG. 182. Efficacy of AFCC on H1N1 WSN-caused mouse death.

FIG. 183. Body weight change caused by AFCC in mice infected with H1N1 (WSN) influenza.

FIG. 184. Body weight change caused with AFCC treatment in mice infected with H1N1 (WSN) influenza.

FIG. 185. Body weight change caused with vehicle treatment in mice infected with H1N1 (WSN) influenza.

FIG. 186. Effect of AFCC on H1N1-caused mouse mortality.

FIG. 187. The average body weight change in mice infected with H1N1 influenza.

FIG. 188. The efficacy of AFOD on H1N1 WSN-caused mouse death.

FIG. 189. The efficacy of AFCC on H1N1 WSN-caused mouse death.

FIG. 190. Body weight changes caused by AFOD or Oseltamivir treatment in mice infected with H1N1 (WSN) influenza.

FIG. 191. Body weight changes caused by AFCC or Oseltamivir treatment in mice infected with H1N1 (WSN) influenza.

FIG. 192. Photos of mouse organs dissected in the end of the study RAAS-201110170.

FIG. 193. Day 1 if HBsAg level.

FIG. 194. Day 3 of HBsAg level.

FIG. 195. Efficacy of therapeutic treatment of prophylactic treatment of RAAS-8 or ETV on in vivo HBV replication in HBV mouse HOI model.

FIG. 196. Effect of prophylactic treatment or therapeutic treatment of RAAS 8 or ETV on the HBsAg in mouse blood.

FIG. 197. Effect of prophylactic treatment or therapeutic treatment of RAAS 8 or ETV on the intermediate HBV replication in the mouse livers by qPCR

FIG. 198. HBV DNA level in plasma effect of treatment or therapeutic treatment of RAAS 8 or ETV.

FIG. 199. Southern blot determination of intermediate HBV DNA in mouse livers.

FIG. 200. The body weights of mice treated with vehicle or indicated compounds during the course of experiment.

FIG. 201. Picture of mouse 4-6 which grew hair on top of head.

FIG. 202. Picture of Fibrin Sealant inhibiting the growth of lung cancer cell.

FIG. 203. Picture of Lung cancer cell without Fibrin Sealant.

FIG. 204. Picture of Lung cancer cell with Fibrin Sealant.

FIG. 205. Picture of lung cancer cells in medium.

FIG. 206. Photos of peripheral nerve repair in Rhesus monkey.

FIG. 207. Photos of peripheral nerve repair in Rhesus monkey.

FIG. 208. Photos of peripheral nerve repair in Rhesus monkey.

FIG. 209. Peripheral nerve degradation and regeneration.

FIG. 210. Nerve conduit repair, goat common peroneal nerve.

FIG. 211. Goat distal nerve immunohistochemical staining.

FIG. 212. Pictures of goat after 7 days of operation and 16 months later.

FIG. 213. Pictures of nerve conduit group 16 months after operation and vehicle control.

FIG. 214. Picture of Goat after 7 days of operation and self graft group 16 months later.

FIG. 215. Picture of nerve conduit group 16 months later and vehicle control

FIG. 216—Picture of FRIII and AFCC KH

FIG. 217 APCC KH

FIG. 218 and FIGS. 219A-D—FRIII Process

FIG. 220—Flow chart OF AFCC 01 process FROM FrIII PASTE

FIG. 221—Flow chart of AFCC02 PROCESS FROM FrIII PASTE

FIG. 222—Flow chart of AFCC03 PROCSS FROM FrIII PASTE

FIG. 223—Flow chart OF AFCC04 FROM FrIII PASTE

FIG. 224—PROCESS OF AFCC05 FROM FrIII PASTE

FIG. 225—Flow chart of AFCC 06 PROCSS FROM FrIII PASTE

FIG. 226—Flow chart of AFCC 07 PROCSS FROM FrIII PASTE

FIG. 227—Flow chart of AFCC 08 PROCSS FROM FrIII PASTE

FIG. 228—Flow chart of AFCC 09 PROCSS FROM FrIII PASTE

FIG. 229—Flow chart of AFCC 10 PROCSS FROM FrIII PASTE

FIG. 230—Flow chart of AFCC 11 PROCSS FROM FrIII PASTE

FIGS. 231A&B—Flow chart of AFCC 12 PROCSS FROM FrIII PASTE

FIG. 232—Flow chart of AFCC 13 PROCSS FROM FrIII PASTE

FIG. 233—Flow chart of AFCC 14 PROCSS FROM FrIII PASTE

FIG. 234—Flow chart of AFCC 15 PROCSS FROM FrIII PASTE

FIG. 235—Flow chart of AFCC 16 PROCSS FROM FrIII PASTE

FIG. 236—AFOD KH & Fr. IV

FIG. 237—AFOD KH

FIGS. 238A-D—Flow chart of AFOD and PCC from FrIV1+IV4 ppt with chromatography method

FIG. 239—Flow chart of AFOD01 FROM FrIV1+IV4 PASTE

FIG. 240—Flow chart of AFOD02 FROM FrIV1+IV4 PASTE

FIG. 241—Flow chart of AFOD03 FROM FrIV1+IV4 PASTE

FIG. 242—Flow chart of AFOD 04 FROM FrIV1+IV4 PASTE

FIG. 243—Flow chart of AFOD 05 FROM FrIV1+IV4 PASTE

FIG. 244—Flow chart of AFOD 06 FROM FrIV1+IV4 PASTE

FIG. 245—Flow chart of AFOD 07 FROM FrIV1+IV4 PASTE

FIG. 246—Flow chart of AFOD 08 FROM FrIV1+IV4 PASTE

FIGS. 247A&B—Flow chart of AFOD 09 FROM FrIV1+IV4 PASTE

FIGS. 248A&B—Flow chart of AFOD 10 FROM FrIV1+IV4 PASTE

FIGS. 249A&B—Flow chart of AFOD 11 FROM FrIV1+IV4 PASTE

FIGS. 250A&B—Flow chart of AFOD 12 FROM FrIV1+IV4 PASTE

FIGS. 251A&B—Flow chart of AFOD 13 FROM FrIV1+IV4 PASTE

FIGS. 252A&B—Flow chart of AFOD 14 FROM FrIV1+IV4 PASTE

FIG. 253—Flow chart of AFOD 15 FROM FrIV1+IV4 PASTE

FIG. 254—Flow chart of AFOD 16 FROM FrIV1+IV4 PASTE

FIGS. 255-265—Photographs of Cryopaste and FVIII

### BACKGROUND

The discovery of the new proteins which are already in existence in all the plasma derived products from human source, animal source, recombinant DNA source, Monoclonal source, transgenic source, natural substance and the expression of cell from the cultured GOOD HEALTHY CELLS lead us to the discovery of a number of the following human plasma process:

HUMAN Blood Plasma

1) AFOD RAAS 101@ contain protein ALB Uncharacterized protein, HPR 31 kDa protein, Albumin Uncharacterized protein, AIBG isoform 1 of Alpha-1B-glycoprotein, all of these proteins can be found in the import human albumin from the three different manufacturers. but lack HPR haptoglobin, ACTC1 Actin, alpha cardiac muscle and KH51 protein which can only be found in AlbuRAAS® and the concentration of Human Albumin containing all these proteins must be equal to 30% or higher to be effective.

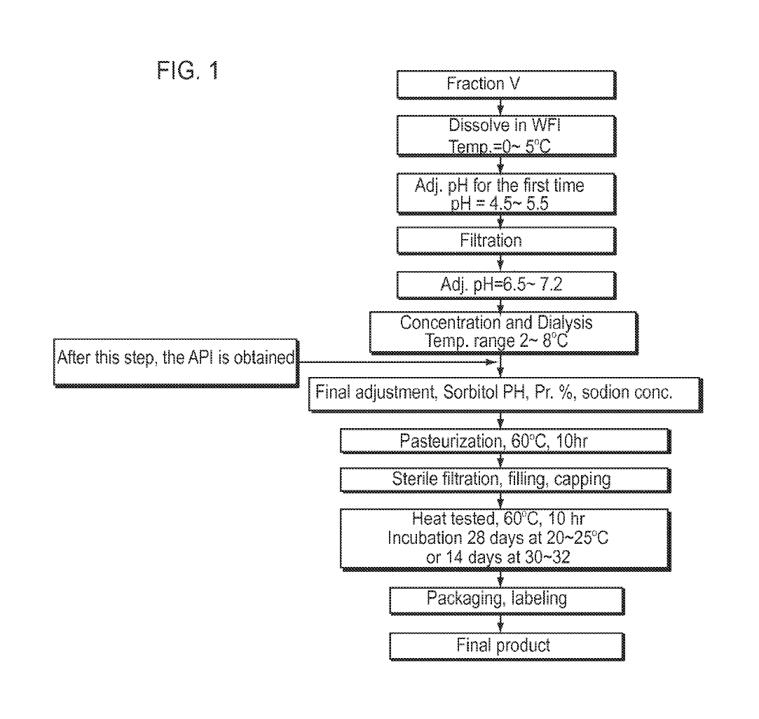

FIG. 1

Protein sequences of ALB Uncharacterized protein, HPR 31 kDa protein, Albumin

Uncharacterized protein, AIBG isoform 1 of Alpha-1B-glycoprotein HPR haptoglobin. Protein sequence of M1, M2, M7, M9, M1O

| | | Instr./Gel Origin | |
|---|---|---|---|
| | 299/m1 | Instrument Sample | |
| | [1] Sample Project | Name | |
| Accession | 20120517 | Protein | |
| No. | Protein Name | Pi | Protein MW |
| PI00022434 | Tax ld, 9606 Gene_Symboi ALB | 6.33 | 738814 |
| | Uncharacterized | | |
| | protein | | |

Peptide Information

| | Obsrv | | | Start | End | |
|---|---|---|---|---|---|---|
| Caic. Mass | Mass | ±da | ±ppm | Seq. | Seq. | Sequence |
| 875.5098 | 875.5258 | 0.016 | 18 | 243 | 249 | LSO.RFPK |
| 927.4934 | 927.5149 | 0.0215 | 23 | 162 | 168 | YLYEIAR |
| 927.4934 | 927.5149 | 0.0215 | 23 | 162 | 168 | YLYE1AR |
| 960.5625 | 960.5834 | 0.0209 | 22 | 427 | 434 | FQNALLVR |
| 960.5625 | 960.5834 | 0.0209 | 22 | 427 | 434 | FQNALLVR |
| 1000.6037 | 1000.612 | 0.0083 | 8 | 550 | 558 | QTALVELVK |
| 1055.5884 | 1055.6189 | 0.0305 | 29 | 161 | 168 | KYLYEAR |
| 1074.5426 | 1074.5758 | 0.0332 | 31 | 206 | 214 | LDELRDEGK |
| 1083.5946 | 1063.62 | 0.0254 | 23 | 162 | 169 | YLYE1ARRq |
| 1128.6987 | 1128.7164 | 0.0177 | 16 | 549 | 558 | KOTALVELVK |
| 1138.498 | 1138.5211 | 0.0231 | 20 | 500 | 508 | CCIESLVNR |
| 1311.7419 | 1311.7593 | 0.0174 | 13 | 362 | 372 | HPDYSVV::!R |
| 1358.6298 | 1358.6437 | 0.0139 | 10 | 570 | 581 | AVMDDFAAFVEK |
| 1358.6298 | 1358.6437 | 0.0139 | 10 | 570 | 581 | AVMDDFAAFVEK |
| 1371.5668 | 1371.5905 | 0.0237 | 17 | 187 | 198 | AAFTECCQAADK |
| 1443.6421 | 1443.6641 | 0.022 | 15 | 287 | 298 | YICENQDSESSK |
| 1467.8431 | 1467.8513 | 0.0082 | 6 | 361 | 372 | RHPDYSWLLLR |
| 1511.8429 | 1511.8691 | 0.0262 | 17 | 439 | 452 | VPQVSIPILVEVSR |
| 1546.7968 | 1546.8112 | 0.0144 | 9 | 299 | 310 | LKECCEKPLLEK |
| 1552.5978 | 1552.62 | 0.0222 | 14 | 384 | 396 | CCAAAD PH |
| | | | | | | ECYAK |
| 1552.5978 | 1552.62 | 0.0222 | 14 | 384 | 396 | CCAAADPHECY |
| | | | | | | AK |
| 1627.6904 | 1627.745 | 0.0546 | 34 | 585 | 598 | ADDKEICFAEEG |
| | | | | | | QK |
| 1639.9379 | 1639.9292 | −0.0087 | −5 | 433 | 452 | KVPQVSTPTLVE |
| | | | | | | VSR |
| 1639.9379 | 1639.9292 | −0.0087 | 5 | 438 | 452 | KVPQVSTPILVE |
| | | | | | | VSR |
| 1650.8949 | 1650.8706 | −0.0243 | −15 | 250 | 264 | AEFAEVSKLVTD |
| | | | | | | LIK |
| 1657.7527 | 1657.7756 | 0.0229 | 14 | 414 | 426 | QNCE I FE QL |
| | | | | | | GEYK |
| 1684.821 | 1684.9177 | 0.0967 | 57 | 287 | 300 | Y10ENQDSISSKLK |
| 1714.7966 | 1714.8048 | 0.0082 | 5 | 118 | 130 | QEPERNECFLQHK |
| 1856.9099 | 1856.8966 | −0.0133 | −7 | 566 | 581 | EQLKAVMDDFA |
| | | | | | | AFVEK |
| 1910.9318 | 1910.9406 | 0.0088 | 5 | 509 | 524 | RPCFSALEVDETYWK |
| 1910.9318 | 1910.9406 | 0.0088 | 5 | 509 | 524 | RPCFSALEVDETYVPK |
| 1996.9294 | 1996.942 | 0.0126 | 6 | 123 | 138 | NECFLQHKDDNPNLPR |
| 2045.0955 | 2045.0938 | −0.0017 | 397 | 413 | | VFDEFKPLVEEPQNLEK |
| 2045.0955 | 2045.0938 | −0.0017 | −1 | 397 | 413 | VEDEFKPLVEEPQNLIK |
| 2124.9875 | 2124.9539 | −0.0336 | 187 | 205 | | AAFTECCQAADKAACLLpK |
| 2260.0227 | 2260.0466 | 0.0239 | 525 | 543 | | EFNAETFTEHADICTLSEK |
| 2545.1665 | 2545.1492 | −0.0173 | 525 | 545 | | EFNAEIFITHADICILSEK |
| | | | | | | ER |
| 2585.1177 | 2585.0925 | −0.0252 | −10 | 265 | 286 | VHIECCHGDLLECADDR |
| | | | | | | ADLAK |
| 2585.1177 | 2585.0925 | −0.0252 | −10 | 265 | 286 | VHIECCHGDLLECADDR |
| | | | | | | ADLAK |
| 2599.2974 | 2599.1685 | −0.1289 | −50 | 414 | 434 | QNCELFEQLGEYKFONA |
| | | | | | | LLVR |
| 2650.2642 | 2650.1511 | −0.1131 | −43 | 139 | 160 | LVRPEVDVNICIAFFEDNE |
| | | | | | | ETFLK |
| 2666.259 | 2666.1682 | −0.0908 | −34 | 139 | 160 | LVRPEVDVMCIAFFEDNE |
| | | | | | | ETFLK |
| 2794.354 | 2794.2439 | −0.1101 | −39 | 139 | 161 | LVRPEVDVNICIAFFEDNE |
| | | | | | | ETFLKK |
| 2794.354 | 2794.2439 | −0.1101 | −39 | 139 | 161 | LVRPEVDVMCIAFFEDNE |
| | | | | | | ETFLKK |

Protein Sequence of M1, M2, M7, M9, M1O

| | | Instr./Gel Origin | |
|---|---|---|---|
| | 300/m2 | Instrument Sample | |
| | [1] Sample Project | Name | |
| Accession | 20120517 | Protein | |
| No. | Protein Name | Pi | Protein MW |
| IPI00431645 | Tax ld 9606 Gene_Symbol | 8.48 | 31673 |
| | f-IPR 31 kDa protein | | |

Peptide Information

| | Obsrv. | | | Start | End | |
|---|---|---|---|---|---|---|
| Calc. Mass | Mass | ±da | ±ppm | Seq. | Seq. | Sequence |
| 809.3788 | 809.368 | −0.0108 | −13 | 146 | 152 | DYAEVGR |
| 920.4625 | 920.4637 | 0.0012 | 1 | 46 | 53 | GSFPV\IQAK |
| 920.4625 | 920.4637 | 0.0012 | 1 | 46 | 53 | GSFPWC)AK |
| 980.4948 | 960.4968 | 0.002 | 2 | 153 | 161 | VGYVSGV\IGR |
| 980.4948 | 980.4968 | 0.002 | 2 | 153 | 161 | VGYVSGWGR |
| 1203.6368 | 1203.6545 | 0.0177 | 15 | 267 | 276 | VT.SEQDWVQK |
| 1290.7305 | 1290.6764 | −.0.0541 | −42 | 91 | 102 | DIAPILTLYVGK |
| 1345.6458 | 1345.6672 | 0.0214 | 16 | 255 | 266 | SCAVAEYGVYVK |
| 1723.8142 | 1723.8369 | 0.0227 | 13 | 173 | 186 | YVNILPVADQDQC!R |
| 1723.3142 | 1723.8369 | 0.0227 | 13 | 173 | 186 | 1 t/MLPVADQDQCIR |
| 1850.9139 | 1850.9366 | 0.0227 | 12 | 137 | 152 | VMPICI_PSKENADIGR |
| 1850.9139 | 1650.9366 | 0.0227 | 12 | 137 | 152 | VMPICIPSKDYABIGR |
| 2172.0576 | 2172.0862 | 0.0286 | 13 | 201 | 220 | SPVGVONLNEHTFCAG |
| | | | | | | MSK |
| 2172.0576 | 2172.0862 | 0.0286 | 13 | 201 | 220 | SPVGVQPILNEHTFCAG |
| | | | | | | MSK |
| 2188.0525 | 2188.0706 | 0.0181 | 8 | 201 | 220 | SPVGVQP1LNEHTFCAG |
| | | | | | | MSK |

| | | Instr./Gel Origin | |
|---|---|---|---|
| | 305/M7 | Instrument Sample | |
| | [1] Sample Project | Name | |
| Accession | 20120517 | Protein | |
| No. | Protein Name | Pi | Protein MW |
| IPI00022434 | Tax ld 9606 Gene_Symbol f\LB | 6.33 | 73881.4 |
| | protein | | |
| | Uncharacterized | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| 927.4934 | 927 4874 | −0.006 | −6 | 162 | 168 | YLYEIAR |
| 927.4934 | 927.48?4 | ··0.006 | ·6 | 162 | 168 | YLYEIAR |
| 960.5625 | 960.5604 | −0.002″1 | −2 | 427 | 434 | FQf\lALLVR |
| 950.5625 | 960.5604 | −0.002″1 | −2 | 427 | 434 | FQNALLVR |
| 1000.60:37 | 1000.5975 | −0.0062 | −6 | 550 | 558 | QTALVELVK |
| ″1055.5884 | 1055.5979 | 0.0095 | 9 | 161 | 168 | KYLYEiAR |
| ″10745426 | 1074.5447 | 0.0021 | 2 | 206 | 214 | LDELRDEGK |
| 1138.498 | 1138.5083 | O.Oi03 | 9 | 500 | 508 | CCTESLVNR |
| 1149.615 | 1149.6238 | 0.0088 | 8 | 66 | 75 | LVI\JEVTEFAK |
| 1311.7419 | 1:311.7579 | 0{116 | 12 | 362 | 372 | HPDYSVVLLLR |
| ″1342.6348 | 1342.6411 | 0.0063 | 5 | 510 | 581 | AVMDDFAAFVE:K |
| 1342.6348 | 1342.6411 | 0.0063 | 5 | 570 | 581 | AVMDDF/V\FVEK |
| 1352.″1686 | 1352.T79l | om·1·1 | 8 | 427 | 437 | FQNALL VRYTK |
| 1358.6298 | 1:358.6348 | 0{105 | 4 | 570 | 581 | AVMDDFi\AFVEK |
| ″137″1.5668 | 1371.5879 | 0.0211 | 15 | 181 | 198 | AAFTECCQAADK |
| 1443.6421 | 1443.6553 | 0.0132 | 9 | 287 | 298 | YICENQDSISSI< |
| 146″1.8431 | 1461.8514 | 0.0143 | ″1O | 36″1 | 31′2 | RHPDYSVVLLLR |
| 1467.84:31 | 14137.8574 | (1.(J143 | 10 | 361 | 372 | RHPDYSVVLLLR |
| ″15″1″1.8429 | 1511.8596 | O.o167 | 11 | 4:9 | 452 | VPOVS |
| | | | | | | TPTLVE:VSR |
| 1546.7968 | 1546.8142 | 0.0174 | 11 | 299 | :310 | LI<EC:CEKPLLEI< |
| 1552.5918 | 1552.6318 | 0.034 | 22 | 384 | 396 | CCAAADPHECYAK |
| 1552.5978 | 1552.13318 | (1.(J34 | 22 | 384 | 396 | CCAAADPHECYAK |
| ″1623.7876 | 1623.8319 | 0.0443 | 2l | :H8 | 360 | DVFLGMFLYE:YAR |
| 1627.6904 | 1627.7493 | 0.0589 | 36 | 585 | 598 | ADDKETC:FAEEG |
| | | | | | | QK |
| 15:9.9319 | 1639.9246 | −0.0133 | −8 | 438 | 452 | KVPQVSTPTLVE:V |
| | | | | | | SR |
| 1639.9:379 | 113:39.92413 | −0.CJ133 | −8 | 438 | 452 | KVPQVSTPTLVEV |
| | | | | | | SR |
| ″1650.8949 | 1650.8693 | 0.0256 | −16 | 250 | 264 | AEFAEVSKLVTDL |
| | | | | | | TK |
| 1657.7527 | 1657.7588 | 0.0061 | 4 | 414 | 4213 | ONCELFEQLGEYK |
| 1684.821 | 1684.8501 | 0.0291 | ″17 | 281′ | 300 | YICEI\JQDSISSKLK |
| 1742.8942 | 1742.91713 | (1.(J234 | 13 | 170 | 183 | HPYFYAPELLFFAK |
| 1898.9952 | 1899.0358 | 0.0406 | 21 | 110 | 184 | HPYFYAPE:LLFFA |
| | | | | | | KR |
| 1898.9952 | 1899.0358 | 0.0406 | 21 | 169 | 183 | RHPYFYAPELLFFA |
| | | | | | | I< |
| 1910.9318 | 1910.9614 | 0.0196 | ″10 | 509 | 524 | RPCFSALEVDE:TY |
| | | | | | | VPK |
| 1910.9:318 | 1910.9514 | 0{1196 | 10 | 509 | 524 | RPCFS,<\LEVDETY |
| | | | | | | VPK |
| ″1924.0863 | 1924.0873 | 0.001 | 1 | 4: 9 | 466 | VPOVSrPTLVE:VS |
| | | | | | | RNL GK |
| 2045.0955 | 2045.0996 | 0.0041 | 2 | 397 | 413 | VFDEFI<PLVEEPQ |
| | | | | | | NLII< |
| 204S.G955 | 2046.0996 | 0.004″1 | 2 | 391′ | 413 | VFDE:FKPLVE:EPO |
| | | | | | | I\JLIK |
| 2086.8:3713 | 20813.81394 | 0{1318 | 15 | 265 | 281 | VHTECC:HGDLLE |
| | | | | | | CADDR |
| 2260.0227 | 2260.0278 | 0.0051 | 2 | 525 | 643 | E:FNAE:TFn=HADI |
| | | | | | | CrL. SEK |
| 2545.1665 | 2545.1123 | −0.0542 | −21 | 525 | 545 | EFNAETFTFHADIC |
| | | | | | | :TLSEK |
| | | | | | | ER |
| 2585.1177 | 2585.1113 | −0{1064 | −2 | 265 | 286 | VHTECC:HGDLLE |
| | | | | | | CADDR |
| | | | | | | ADLAK |
| 2585.1177 | 2585.1113 | −0{1064 | −2 | 265 | 286 | VHTECC:HGDLLE |
| | | | | | | CADDR |
| | | | | | | ADLAK |
| 2599.2974 | 2599.0598 | −0.2376 | −91 | 414 | 4:34 | ONCELFEQL |
| | | | | | | GEYKFQNA |
| | | | | | | LLVR |
| 2650.21343 | 21350.21305 | −0.CJ037 | −1 | 139 | 160 | LVRPEVDVMCTi\F |
| | | | | | | HDNE |
| | | | | | | ElH.K |
| 2778.3589 | 2778.3564 | −0.0025 | −1 | 139 | 1131 | LVRPEVDVMCTAF |
| | | | | | | HDNE |
| | | | | | | ETFLKK |
| 2794.354 | 2794.3438 | −0{1102 | −4 | 139 | 161 | LVRPEVDVMCTi\F |
| | | | | | | HDNE |

| | | Instr./Gel Origin | |
|---|---|---|---|
| | 307/M9 | Instrument Sample | |
| | [1] Sample Project | Name | |
| Accession | 20120517 | Protein | Protein |
| No. | Protein Name | Pi | MW |
| IPI00022895 | Tax ld = 9606 Gene Symboi = A18G | 5.56 | 5478B.8 |
| | lsofoml1 of | | |
| | Alpha-1 B-glycoprotein | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| 861.46?6 | 86•1.4217 | −0.0459 | −53 | 437 | 444 | EGETKAVK |
| 870.5295 | 870.5177 | −0{1118 | −14 | 107 | 114 | LLELTGPK |
| 8705295 | 8l0.51T7 | −0.0118 | −14 | 10? | 114 | LLEL rGPK |
| 1264.6532 | 126413721 | 0.0189 | 15 | 95 | 106 | SGLSTGWTQ |
| | | | | | | LSK |
| 1264.65: 2 | •1264.6721 | O.o-!89 | 15 | 95 | 106 | SGLSTGWTO |
| | | | | | | LSK |
| 1372.6969 | 1372.7217 | (1.(J248 | 18 | 79 | 90 | HQFLLTGDT |
| | | | | | | QGR |
| “1372.6969 | 1:r12.1211 | 0.0248 | 18 | 79 | 90 | HQFLLTGDlT |
| | | | | | | GR |

| | | Instr./Gel Origin | |
|---|---|---|---|
| | 308/M10 | Instrument Sample | |
| | [1] Sample Project | Name | |
| Accession | 20120517 | Protein | |
| No. | Protein Name | Pi | Protein MW |
| IPI00641737 | Tax--ld = 9606 Gene | 6.13 | 45860.8 |
| | Symboi = HP; HPR Haptoglobin | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| 856.4675 | 856.4838 | 0.0163 | 19 | 113 | 118 | NYYKLR |
| 855.4615 | 856.4lB8 | 0.0163 | 19 | 54 | 59 | NYYKLR |
| 920.4625 | 920.4198 | −0.0427 | −46 | 171 | 178 | GSFPWQAK |
| ·1 708.850·1 | 1708.8895 | 0.0394 | 23 | 111 | “!3”! | LRTEGDGVYTLNN |
| | | | | | | EK |
| 1857.9198 | 1857.9403 | 0.0205 | 11 | 137 | 153 | AVGDKLPECEAVC |
| | | | | | | GKPK |
| 185?.9198 | 185l.94tn | 0.0205 | 11 | ″137 | 153 | AVGDKL |
| | | | | | | PECEAVCGKPK |

In the final comparison AFOD RAAS 101 product contains a total of six proteins ALB Uncharacterized protein, HPR 31 kDa protein, Albumin Uncharacterized protein, A1BG isoform 1 of Alpha-1B glycoprotein HPR haptoglobin and KH51. In this product it contains HPR Haptoglobulin, ACTC1 Actin, alpha cardiac muscle 1 and a newfound protein KH51 both of which are very crucial in the application for cancer and bacteria. These three proteins could not be found in any international imported human albumin.

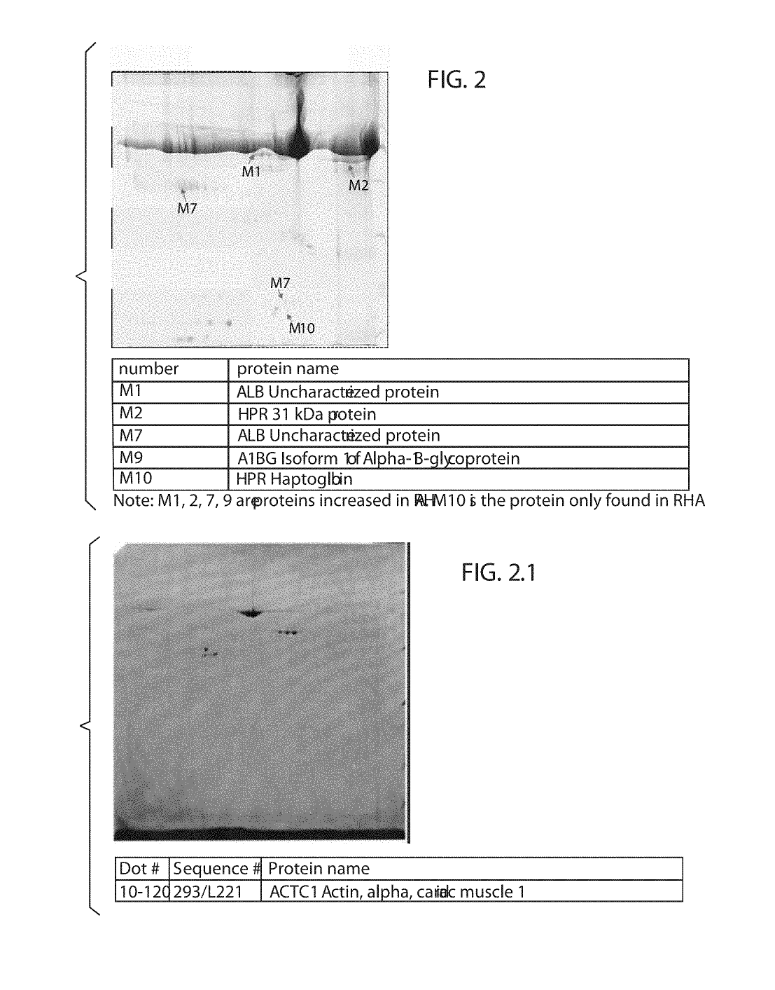

FIG. 2, 2.1

To compare with AFOD RAAS 101 international import company 1 has only one protein HPR 31 kDa

Protein vs 7 proteins in AFOD RAAS 101.

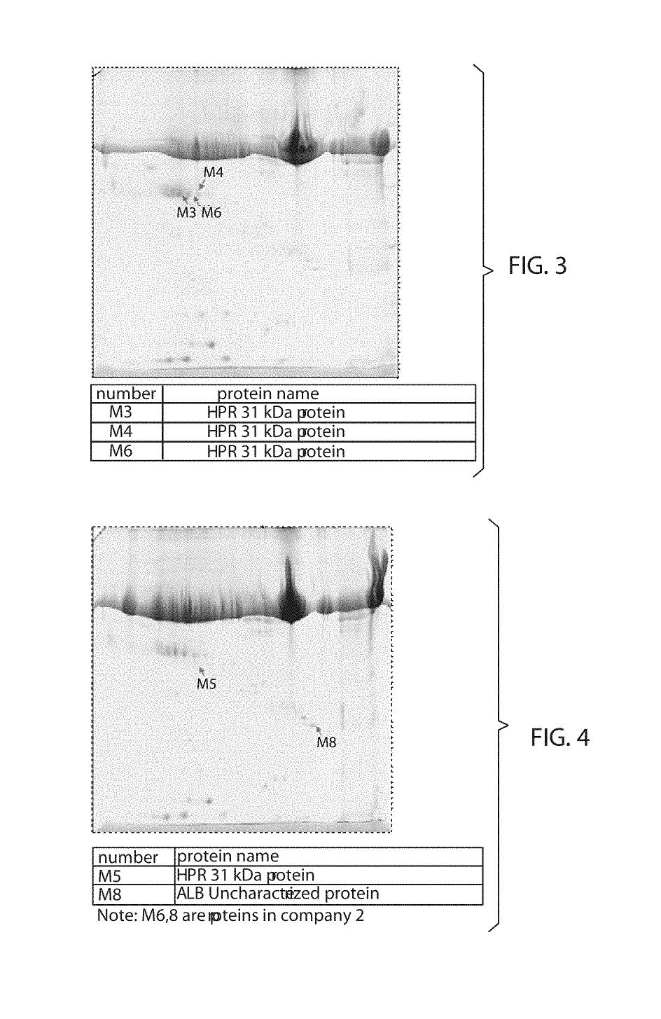

FIG. 3

Company 2 has two proteins HPR 31 kDa and Albumin uncharacterized proteins vs 7 proteins in AFOD RAAS 101.

FIG. 4

Company 3 has three proteins Albumin uncharacterized protein, HPR 31 kDa protein and, A1BG isoform

1 of Alpha-1B-glycoprotein vs 7 proteins in AFOD RAAS 101.

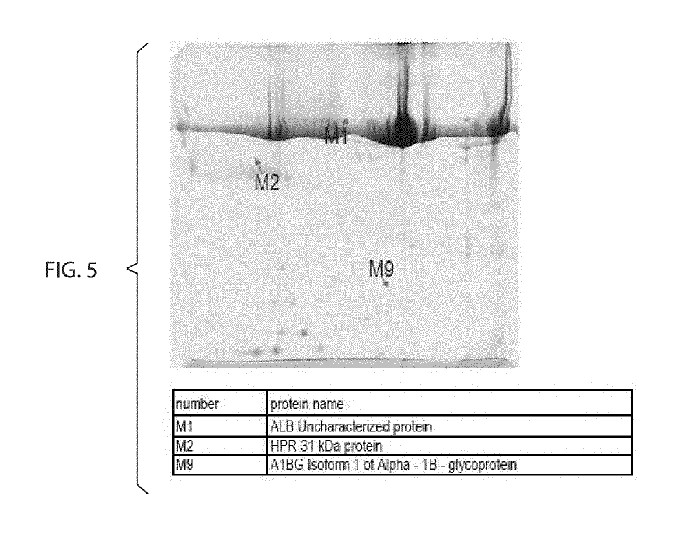

FIG. 5

In conclusion the maximum amount of proteins in the international import companies is three or 58% LESS compared to AFOD RAAS 101, and the minimum amount of proteins is one protein or 86% LESS. None of the international import companies contain the existing protein HPR Heptaglobulin, ACTC1

Actin, alpha cardiac muscle 1 and new discovered KH51 protein.

2) AFOD RAAS 102®: Beside the main component of Immunoglobulin AFOD RAAS 102 contains three existing proteins 120/E19 IGHV4-31; IGHG144 kDa protein and 191/H18 IGHV4•31; IGHG1

32 kDa and IGHV4•31; 1GHG1 Putative uncharacterized protein DKFZp686G11190 proteins including five newly discovered proteins KH33, KH34, KH35, KH36 and KH37. The combination of these five proteins with the concentration at 30% have been found to be very effective against the viruses like H1N1, H5N1, foot and mouth disease and specially changing the protein which causes the Hepatitis B virus to stop the DNA replication and cure the Hepatitis B within the three days in mice and as well as bacteria and solid and blood cancers.

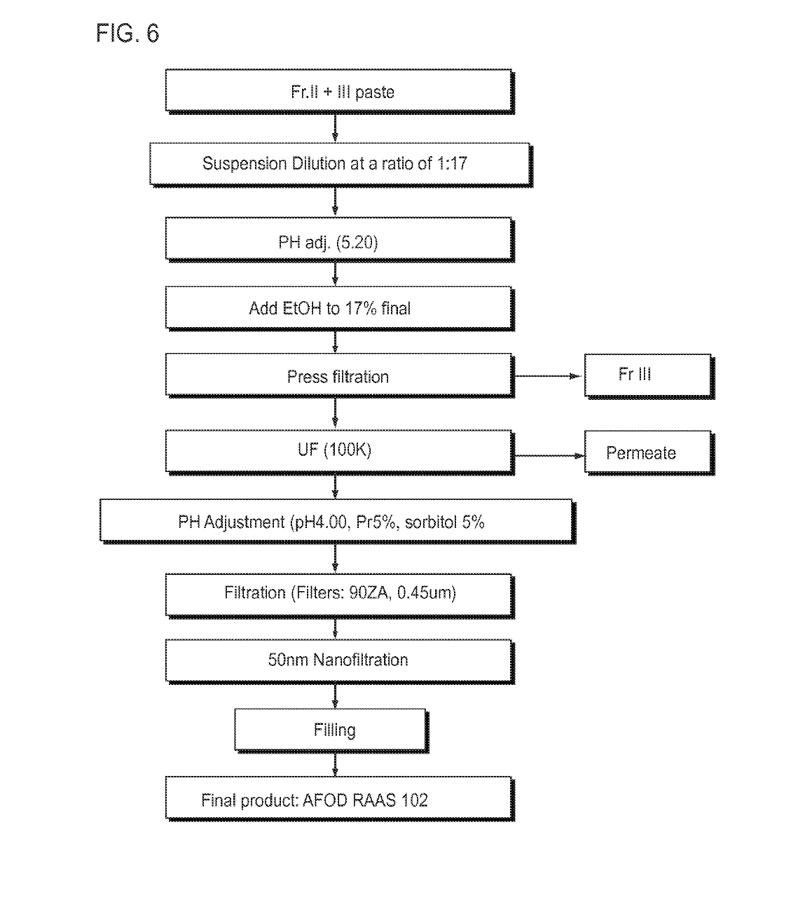

FIG. 6

Protein sequence

| | 120E19 | Instr./Gel Origin | |
|---|---|---|---|
| | [1] Sample Project | Instrument Sample Name | |
| Accession | 2012 Jun. 14 | Protein | Protein |
| No. | Protein Name | Pi | MW |
| IPI00448925 | Tax_!d, %06 | 6.55 | 44511.3 |
| | Gene_Syrnboi, IGHV4 · 31; | | |
| | 1GHG1 44 kDa | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| 835.4342 | 835.4091 | −0.02′\1 | −30 | 132 | 138 | DTLMISR |
| 838.5032 | 838.4759 | −0 0273 | ··33 | 210 | 217 | ALPf\PIEK |
| 838.5032 | 838.4759 | ··0.0273 | −33 | 210 | 217 | ALPAPIEK |
| 851.4291 | 851.4036 | −0.0255 | −30 | 132 | 138 | DTLM!SR |
| 1161.6296 | 1161.6327 | 0.0031 | 3 | 244 | 253 | NQVSLTCLVK |
| 1161.62% | 1161.6327 | o.orn1 | 3 | 244 | 253 | NQVSLTCLVK |
| 1186.6467 | 1186.5533 | −0 0934 | −79 | 5 | 1 t) | GPSVFPLAPSSK |
| 1266.674 | 1286.6965 | 0.0225 | 17 | 228 | 238 | EPQVYTLPPSR |
| 1286.674 | 1286.6965 | 0.0225 | 17 | 228 | 238 | EPQVYTLPPSR |
| 1676.8-125 | 1676.9005 | 0.058 | 35 | 385 | 399 | QT!IPDYRr |
| | | | | | | MIGQGA |
| 1677.802 | ″1677.8694 | 0.0674 | 40 | 158 | 171 | FI′JWYVDGVEVH |
| | | | | | | I′JAK |
| 1677.802 | 1677.8694 | 0.0674 | 40 | 158 | 171 | FtNv′YVDGVEVHt |
| | | | | | | AK |
| 1872.9702 | 18″130851 | 0.1149 | 61 | 228 | 243 | EPQVYTLPPSRDE |
| | | | | | | LTK |
| 1872.9702 | 1873.0851 | 0.1149 | 61 | 228 | 243 | EPQVYTLPPSRDE |
| | | | | | | LTK |
| 2139.027621 | 2139.0417213 | 0.01410.199 | 7 | 139 | 1571 | TPEVTCVVVDVS |
| | | | | | | HEDPET |
| | | | | | | VK |
| 9.0276 | 9.22″11 | 5 | 93 | 139 | 157 | TPEVTCVVVDVS |
| | | | | | | HEDPE |
| | | | | | | VK |
| 2139.0276 | 2139.22″11 | 0.1995 | B3 | 139 | 15″7 | TPEVTCVvVDVS |
| | | | | | | HEDPE |
| | | | | | | VK |
| 2544.1313 | 2544.37″16 | 0.2403 | 94 | 254 | 275 | GFYPSDIAVEWE |
| | | | | | | SNGQP |
| | | | | | | ENNYK |
| 2801.2671 | 2801.4607 | 0.1936 | 69 | 00 | 22 | WQQGI′JVFSCS\Ir |
| | | | | | | v1HEAL |
| | | | | | | HNHYTQK |
| 2817.622 | 2817.5144 | 0.2522 | 90 | 300 | 32 | WQQGNVFSCSV |
| | | | | | | MHEAL |

| | 191H18 | Instr./Gel Origin | |
|---|---|---|---|
| | [1] Sample Project | Instrument Sample Name | |
| Accession | 20120614 | Protein | |
| No. | Protein Name | Pi | Protein MW |
| IPI00892671 | Tax ld,9606 Gene_Symboi= | 8.3 | 32476.2 |
| | IGHV4-31;IGHG1 32kDa | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| ·J9W.9318 | 1910.9406 | 0.0088 | s | 5G9 | 524 | RPCFSAL.EVDETYVPK |
| 1910.9318 | 1910.9406 | 0.0088 | 5 | 509 | 524 | RPCFSALEVDETYVPK |
| 1996.9294 | 1996.942 | 0.0″126 | 6 | ″!23 | 138 | NECFLQHKDDNPNLPR |
| 2045.0955 | 2045{1938 | −0{1017 | −1 | 397 | 413 | VFDEFKPLVEEPQNLIK |
| 2045.0955 | 2045.0938 | −0.001? | −1 | : 91 | 413 | VFDEFKPLVEEPQNLIK |
| 2124.9875 | 2124.9539 | −0.0336 | −16 | 187 | 205 | A/>.FTECCQ,<\ADKAA |
| | | | | | | CLLP |
| | | | | | | K |
| 2260.0227 | 22130{1466 | (1.(J239 | 11 | 525 | 543 | EFNAETFTFHAD!CTLS |
| | | | | | | EK |
| 2545:1665 | 2545.1492 | −0.01?3 | −l | 525 | 545 | EFNAETFn=HJI.DiCrL. |
| | | | | | | SEK |
| | | | | | | ER |
| 2585.1111 | 2585.0925 | −0.0252 | -W | 265 | 286 | VHrECCHGDLLECAD |
| | | | | | | DR |
| | | | | | | ADLAK |
| 2585.″! | 25850925 | −0.0252 | −10 | 265 | 286 | VHT |
| 11′1′ | | | | | | ECCHGDLLECADDR |
| | | | | | | ADLAK |
| 2599.2914 | 2599.1685 | −0.1289 | −50 | 4″14 | 434 | QNCELFEQLGEYKFQ |
| | | | | | | NA |
| | | | | | | LLVR |
| 2650.2642 | 2650.1511 | −0.1131 | −4: | 1: 9 | 160 | LVRPEVDVMCTAFHD |
| | | | | | | NE |
| | | | | | | ETFLI< |
| 2666.259 | 2666.1682 | −0.0908 | −34 | ″!39 | 160 | LVRPEVDVMCrAFHD |
| | | | | | | NE |
| | | | | | | ETFLK |
| 2794.354 | 2794.2439 | −0.1101 | < 9 | 1: 9 | 161 | LVRPEVDVMCTAFHD |
| | | | | | | NE |
| | | | | | | ETFLI<K |
| 2?94.: 54 | 2194.2439 | −0.1″10″1 | −39 | ″!39 | 161 | LVRPEVDVMCrAFHD |
| | | | | | | NE |
| | | | | | | ETFLKI< |
| 1161.6296 | 1161.6295 | ··0.0001 | 0 | 209 | 218 | NQVSLTCLVK |
| 1161.6296 | ″1161.6295 | −0.0001 | 0 | 209 | 218 | NQVSLTCLVK |
| 1286674 | 1286.6779 | 0.0039 | 3 | 193 | 203 | EPQVYTLF′PSR |
| 1286.674 | 1286.6779 | 0.0039 | 3 | 193 | 203 | EPQVYTLPPSR |
| 18n.9″?02 | 1872.993″1 | 0.0 35 | 13 | 193 | 208 | EPQ\/YTLPPSRDELTK |
| 1872.9702 | 1872.9937 | 0.0235 | 13 | 193 | 208 | EPQVYTLPPSRDELTK |
| 18″?3.9219 | 1873.9736 | 0.0517 | 28 | 241 | 257 | TTPPVLDSDGSFFLYSK |
| 2544.1313 | 2544.1079 | −0.0234 | −9 | 219 | 240 | GFYPSDIAVEWESI′JG |
| | | | | | | QP EI′JI′JYK |
| 2544.B13 | 2544.10″?9 | −0.0234 | −9 | 219 | 240 | GFYPSDIAVEWESI′JG |
| | | | | | | QP |
| | | | | | | ENNYK |
| 2801.2671 | 2801.2739 | 0.0068 | 2 | 26 ) | 28? | WOQGI′JVFSCSVMHE |
| | | | | | | AL HNHYTQK |
| 2801.2671 | 2801.2739 | 0.0068 | 2 | 265 | 287 | WOQGNVFSCSVI\JI |
| | | | | | | HEAL |
| 2801.2739 | | | | | | |
| | | | | | | HNHYTQK |
| 2817.2622 | 2817.2522 | −0.01 | −4 | 265 | 287 | WQQGr |
| | | | | | | VFSCSVMHEAL Hr |
| | | | | | | HYTQK |

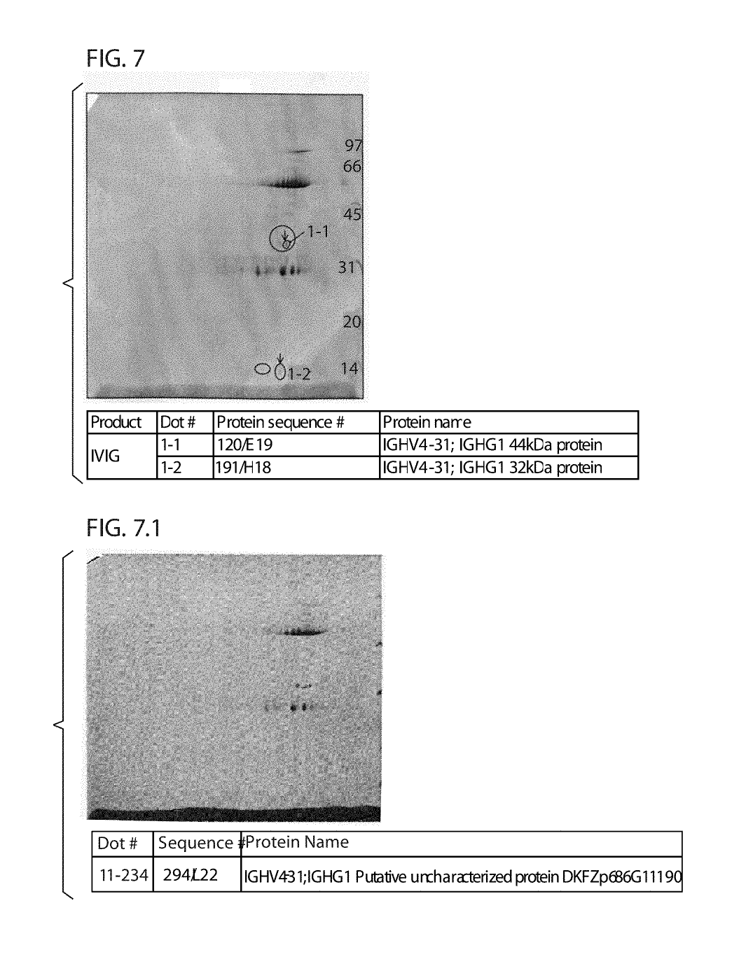

FIG. 7, 7.1

3) AFOD RAAS 103® Contains the four existing discovered proteins 193/H20 TF serotransferrin,

194/H21APOH beta2-glycoprotein 1, 195/H22 eDNA FU5165, moderately similar to beta-2-glycoprotein, 196/H23 FCN3 isoform 1 of Ficolin-3. In addition it may contain KH3, KH4, KHS, KH6, KH7, KH8, KH9, KH10, KH41, KH42 and KH43 proteins. This AFOD RAAS 103 has proven to change the bad protein of the HCV RNA virus into the good protein to cure Hepatitis C.

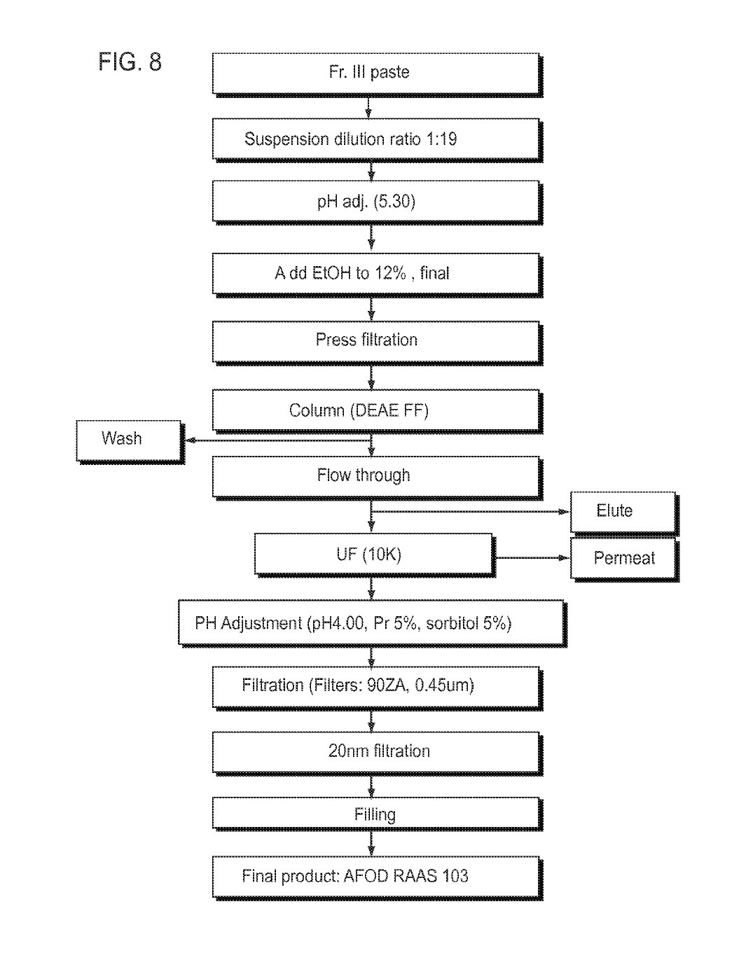

FIG. 8

Protein sequence

| | 193/H20 | Instr./Gel Origin | |
|---|---|---|---|
| | [1] Sample Project | Instrument Sample Name | |
| Accession | 20120614 | Protein | |
| No. | Protein Name | Pi | Protein MW |
| IP100022463 | Tax id=9606 Gene Symboi=TF | 6.81 | 79294.5 |
| | Serotransferrin | | |
| | protein | | |

Peptide Information

| | Obsrv. | | | Start | End | |
|---|---|---|---|---|---|---|
| Calc. Mass | Mass | ±da | ±pp, | Seq. | Seq | Sequence |
| 827.4046 | 827.4172 | 0.0′126 | 15 | 565 | 57′i | r PDPWAK |
| 8″?4.4417 | 874.446B | 0.0052 | 6 | 31 t) | 323 | DSAHGFLK |
| 887.4152 | 887.4246 | O.OOB4 | 11 | 468 | 475 | SCHTf\VGR |
| 964.5323 | 964.5367 | 0.0044 | 5 | 601 | 609 | APNHAV\ITR |
| 997.4771 | 997.4792 | 0.0021 | 2 | 6L″ | 69 | ASYLDCIR |
| 1000.4985 | 1000.4951 | −0 0034 | −3 | 669 | 676 | YLGEEYVK |
| 1015.5101 | 1015.5131 | 0.003 | 3 | 467 | 475 | KSCHT.AVGR |
| 1125.5721 | 1125.5751 | 0.003 | | 61 | 69 | KASYLDCIR |
| 1166.5913 | 1166.5861 | −0.0052 | −4 | 554 | 564 | HQTVPQt TGGK |
| 1195.542t) | 1195.5465 | 0.0039 | 3 | 363 | 3′71 | WCALSHHER |
| 1195.5525 | 1195.5465 | −0.006 | −5 | 123 | 132 | DSGFQMNQLR |
| 1211.5474 | 1211.5527 | 0.0053 | 4 | 123 | 132 | DSGFQIVlNQLR |
| 1249.606 | ″1249.6086 | 0.0026 | .″:. | −154 | −164 | SASDLTWDNLK |
| 1249.606 | 1249.6086 | 0.0026 | 2 | 454 | 464 | SASDLTWDI′JLK |
| 1273.65% | 1273.6465 | −0.0071 | −6 | 226 | 236 | HSTiFENLANK |
| 12″ Lactate | 1 76.6421 | 0.01 | 8 | 300 | 310 | EFQLFSSPHGK |
| dehydrogenase | | | | | | |
| 6321 | | | | | | |
| 1283.5692 | 1283.5695 | 0.0003 | 0 | 531 | 541 | EGYYGYTGAFR |
| 1283.5692 | 1283.5695 | 0.0003 | 0 | 531 | 541 | EGY′r″GYTGAFR |
| 1317.5892 | 1317.5931 | 0.0039 | 3 | 27 | 37 | WCAVSEHEATK |
| 1323.6475 | ″1323.6637 | 0.0162 | 1.″:. | 122 | 132 | KDSGFQMNQLR |
| 13 9.6423 | 1339.6395 | −0.0028 | −2 | 122 | 132 | KDSGFQMI′JQLR |
| 1354.6307 | 1354.6305 | −0.0002 | 0 | 577 | 587 | DYELLCLDGTR |
| 13″?1.7009 | 1377.699 | −0.0017 | −1 | 453 | 464 | KSASDLWVDNl.K |
| 1415.72 | 1415.7227 | 0.0027 | 2 | 47 | 60 | SVIPSDGPSVACVK |
| 1478.73-19 | ″1478.7483 | 0.0134 | g | 332 | 343 | MYLGYEYVTAIR |
| 1491″159 | 1491.7654 | 0.0064 | 4 | 298 | 3′10 | SKEFQLFSSPHGK |
| 1491.759 | 1491.7654 | 0.0064 | 4 | 298 | 10 | SKEFQLFSSPHGK |
| 1494.7297 | 1494.7448 | 0.0151 | 10 | 332 | 343 | MYLGYE′\″VTAIR |
| 1521.7367 | 1521.7344 | ··0.0023 | ·2 | 372 | 384 | LKCDEWSVNSVGK |
| 1531.688 | 1531.7039 | 0.0159 | 10 | 684 | 696 | CSTSSLLEACTFR |
| 1531.688 | 1531.7039 | 0.0159 | 10 | 684 | 696 | CSTSSLLEACTFR |
| 1539.7″108 | ″1539.7297 | 0.0189 | 1.″:. | 240 | 251 | DQYELLCLDI′JTR |
| 1565.7992 | 1565.8019 | 0.0027 | 2 | 647 | 659 | DLLFRDDTVCL!-\K |
| 1565.7992 | 1565.8019 | 0.0027 | 2 | 647 | 659 | DLLFRDDTVCLAK |
| 1577.6577 | 1577.699 | 0.0413 | 26 | 495 | 508 | FDEFFSEGCAPGSK |
| 1586.7744 | 1586.787 | 0.0126 | 8 | 588 | 600 | KPVEEYANCHLAR |
| 1 ′\86.?744 | 1 ′i86.l87 | 0.0126 | 8 | 588 | 600 | KPVEEYANGHLAR |
| 1593.8094 | 1593.7748 | −0.0346 | 22 | 47″t) | 489 | TAGWNIPMGLLYNK |
| 1615.8187 | 1615.8096 | −·0.0091 | −6 | 226 | 239 | HSTIFENL!-\NKADR |
| 162Sl.8159 | 162Sl.799 | −0.0169 | −10 | 108 | 121 | EDPOTFYYAVAVVK |
| 1659.783 | 1659.7869 | 0.0039 | 2 | 683 | 6Sl6 | KCSTSSLLEACTFR |
| 1689.849 | 1689.8651 | 0.0161 | 10 | 259 | 27, | DCHLAQVPSHTVVAR |
| | | | | | ′J, | |
| 1705.7″527 | 1?05.7793 | 0.0 66 | 16 | 4% | 509 | FDEFFSEGCAPGSi\K |
| 1?06.?659 | 1706.7622 | −0.003′7 | 2 | 516 | 530 | LCMGSGLNLCEPNNi\ |
| 1725.767 | 1725.7515 | −00155 | −9 | 385 | 399 | IEGVSAETTEDGIAK |
| 1817.8044 | 1817.7971 | −·0.0073 | −4 | 347 | 362 | EGTCPEAPTDECKPVK |
| 1881.876 | ″1881.88″12 | 0.0052 | 3 | 237 | 251 | ADRDQYELLCLDI′JTR |
| ·:sa·:.876 | 1881.8812 | 0.0052 | 3 | 237 | 251 | ADRDQYELLCLDt TR |
| 1952.9382 | 1952.9524 | 0.0142 | 7 | 572 | 587 | NLNEKDYELLCLDGTR |
| 2549 293 | 2549.3508 | 0.0578 | 3 | 252 | 273 | KPVDEYi\DCHL.AQVPSH |
| | | | | | | TVVAR |
| 19W.9318 | 1910.9406 | 0.0088 | s | SG9 | 524 | RPCFSAL.EVDETYVPK |
| 1910.9318 | 1910.9406 | 0.0088 | 5 | 509 | 524 | RPCFSALEVDETYVPK |
| 1996.9294 | 1996.942 | 0.0″126 | 6 | ″!23 | 138 | NECFLQHKDDNPNLPR |
| 2045.0955 | 2045{1938 | −0{1017 | −1 | 397 | 413 | VFDEFKPLVEEPQNLIK |
| 2045.0955 | 2045.0938 | −0.001? | −1 | : 91 | 413 | VFDEFKPLVEEPQNLIK |
| 2124.9875 | 2124.9539 | −0.0336 | −16 | 187 | 205 | A/>.FTECCQ,<\ADKAACL |
| | | | | | | LP K |
| 2260.0227 | 22130{1466 | (1.(J239 | 11 | 525 | 543 | EFNAETFTFHAD!CTLSEK |
| 2545:1665 | 2545.1492 | −0.01?3 | −l | 525 | 545 | EFNAETFn=HJI.DiCrL.SE |
| | | | | | | K ER |
| 2585.1111 | 2585.0925 | −0.0252 | -W | 265 | 286 | VHrECCHGDLLECADDR |
| | | | | | | ADLAK |
| 2585.″! 11′1′ | 25850925 | −0.0252 | −10 | 265 | 286 | VHT ECCHGDLLECADDR |
| | | | | | | ADLAK |
| 2599.2914 | 2599.1685 | −0.1289 | −50 | 4″14 | 434 | QNCELFEQLGEYKFQNA |
| | | | | | | LLVR |
| 2650.2642 | 2650.1511 | −0.1131 | −4: | 1: 9 | 160 | LVRPEVDVMCTAFHDNE |
| | | | | | | ETFLI< |
| 2666.259 | 2666.1682 | −0.0908 | −34 | ″!39 | 160 | LVRPEVDVMCrAFHDNE |
| | | | | | | ETFLK |
| 2794.354 | 2794.2439 | −0.1101 | < 9 | 1: 9 | 161 | LVRPEVDVMCTAFHDNE |
| | | | | | | ETFLI<K |
| 2794.: 54 | 2194.2439 | −0.1″10″1 | −39 | ″!39 | 161 | LVRPEVDVMCrAFHDNE |
| | | | | | | ETFLKI< |

Instr./Gel Origin

| | | Instr./Gel Origin | |
|---|---|---|---|
| | 194H21 | Instrument | |
| | [1] Sample Project | Sample Name | |
| Accession | 20120614 | Protein | Protein |
| No. | Protein Name | Pi | MW |
| IPI00298828 | Tax_id 9606 Gene_Symboi,APOH | 8.34 | 39584.1 |
| | Beta--2-giycoprolein | | |
| | protein | | |

Peptide Information

| Calc. | Obsrv. | | | Start | End | |
|---|---|---|---|---|---|---|
| Mass | Mass | ±da | ±pp, | Seq. | Seq | Sequence |
| 1022.5266 | 1022.5289 | 0.0023 | 2 | Seq. 271 | Seq. 279 | ATv′VYQGER |
| 1022.5266 | ″1022.528Sl | 0.0023 | ″— | 271 | 279 | ATVVYQGER |
| 1104.5472 | 1104.5469 | −O.OOOi | 0 | 328 | 3% | EHSSLAFWK |
| 1104.5472 | 1104.5469 | −0.0003 | 0 | i28 | 36 | EHSSLAFWK |
| 1150.6216 | 1150.61″?6 | −0.004 | −3 | 2′70 | 2′79 | KATVVYQGER |
| 1502.7784 | 1502.7891 | 0.0107 | 7 | 83 | 96 | VCPFAGILENGAVR |
| 1502.7784 | 1502.7891 | 0.0107 | 7 | 83 | 96 | VCPFAGILENGAVR |
| 1914.0042 | 1913.9966 | −0.0076 | −4 | 2L″ | 38 | TCPKPDDLPFSTVVPLK |
| 1914.0042 | ″1Sl13.9966 | −0.0076 | −4 | 22 | 38 | TCPKPDDLPFSTVVPLK |
| 2085.9104 | 2085.8286 | −0.0818 | −39 | 307 | 324 | CSYTEDAQCIDGTiEVPK |
| 2383.0911 | 2383.1409 | 0.0498 | 21 | 39 | 58 | TFYEPGEEITYSGKPGYV SR |
| 2383.0911 | 2383.1409 | 0.0498 | 21 | 39 | 58 | TFYEPGEEITYSCKPGYV |
| | | | | | | SR |
| 2385.9963 | 2386.1001 | 0.1038 | 44 | 230 | 250 | ATFGCHDGYSLDGPEEiE |
| | | | | | | CTK |
| 2731.3337 | 2731.426 | 0.0923 | 34 | 205 | 227 | GPFPSRPDNGFVNYPAK |
| | | | | | | PTLYYK |

| | | Instr./Gel Origin | |
|---|---|---|---|
| | 195/H22 | Instrument | |
| | [1] Sample Project | Sample Name | |
| Accession | 20120614 | Protein | Protein |
| No. | Protein Name | Pi | MW |
| PI00910625 | Tax id=9606 Gene Symbol=-eDNA | 8.19 | i1402.2 |
| | FLJ51265, | | |
| | moderately similar to Beta-2- | | |
| | glycoprotein | | |

Peptide Information

| Calc. | Obsrv. | | | Start | End | |
|---|---|---|---|---|---|---|
| Mass | Mass | ±da | ±pp, | Seq. | Seq | Sequence |
| 1022.5266 | 1022.5208 | −.0.0058 | −6 | 200 | 208 | ATVVYQGER |
| 1022.5266 | 1022.5208 | −0 0058 | −6 | 200 | 208 | ATV\IYQGER |
| 1104.5472 | 1104.5475 | 0.0003 | 0 | 257 | 265 | EHSSLAFWK |
| 1104.5472 | 1104.5475 | 0.0003 | 0 | 257 | 265 | EHSSLAFWK |
| 1150.6216 | 1150.6241 | 0.0025 | 2 | 199 | 208 | KATVVYQGER |
| 1′\02.″1784 | 1502.8273 | 0.0489 | 33 | 83 | 96 | VCPFAGILENGAVR |
| 1502.7784 | 1502.8273 | 0.0489 | 33 | 83 | 96 | \ICPFAGILENGAVR |
| 1914.0042 | 1914075 | 0.0708 | 37 | 22 | 38 | TCPKPDDLPFSTV\IPLK |
| 1914.0042 | 1914.075 | 0.0708 | 37 | 22 | 38 | TCPKPDDLPFST\IVPLK |
| 2085.Sl104 | 2085.9956 | 0.0852 | 4″1 | 236 | 253 | CSYTEDAQCIDGTiEVPK |
| 2383.0911 | 2383.2917 | 0.2006 | 84 | 39 | 58 | TFYEPGEEITYSCKPGY\1 |
| | | | | | | SR |
| 2383.0911 | 2383.2917 | 0.2006 | 84 | 39 | 58 | TFYEPGEEITYSGKPGYV |
| | | | | | | SR |

Ficoiin-3

| | | Instr./Gel Origin | |
|---|---|---|---|
| | 196/H23 | Instrument | |
| | [1] Sample Project | Sample Name | |
| Accession | 20120614 | Protein | |
| No. | Protein Name | Pi | Protein MW |
| IPI00293925 | Tax_id 9606 Gene_Symboi,FCN3 | 6.2 | 33395.2 |
| | lsoform 1 of | | |
| | Ficoiin-3 | | |

Peptide Information

| Calc. | Obsrv. | | | Start | End | |
|---|---|---|---|---|---|---|
| Mass | Mass | ±da | ±pp, | Seq. | Seq | Sequence |
| 941.5064 | Sl41.4953 | −0.0111 | −12 | 286 | 293 | GVGHPYRR |
| 1024.4846 | 1024.4824 | −0.0022 | −2 | 27? | 28) | YGIDWASGR |
| 1024.4846 | 1024.4824 | ..0.0022 | −2 | 277 | 285 | YGIDWASGR |
| 1046.5265 | 1046.5337 | 00072 | 7 | 267 | 276 | Y!−\VSE!−\1-\AHK |
| 1070.4902 | 1070.486 | −0.0042 | −4 | ″137 | ″145 | QDGSVDFFR |
| 1070.4902 | 1070.486 | −0.00-12 | --1 | 137 | 145 | QDGSVDFFR |
| 1113.5-176 | 111i.54i6 | −0.004 | −4 | 191 | 199 | TFAHYATFR |
| 1113.5476 | 1113.5436 | −0.004 | −4 | 1B1 | 1B9 | TFAHYATFR |
| 1166.6165 | 1166.5963 | −0.002 | −17 | (′″) | g,r) | GEPGDPVNLLR |
| 1226.5913 | 1226.5856 | ..0.0057 | ..5 | 136 | 145 | RQDGSVDFFR |
| 1226.5913 | 1226.5856 | −0 0057 | −5 | 136 | 145 | RQDGSVDFFR |
| 1555.8-179 | 1555.8181 | −0.0298 | −19 | 200 | 213 | LLGEVDHYQLALGK |
| 1555.8479 | 1555.8181 | −0.0298 | −.:S1 | 200 | 213 | LLGEVDHYQLALGK |
| 15B5.821 | 1595.7993 | −0.0217 | −14 | 71 | 85 | MGPKGEPGDPVNLLR |

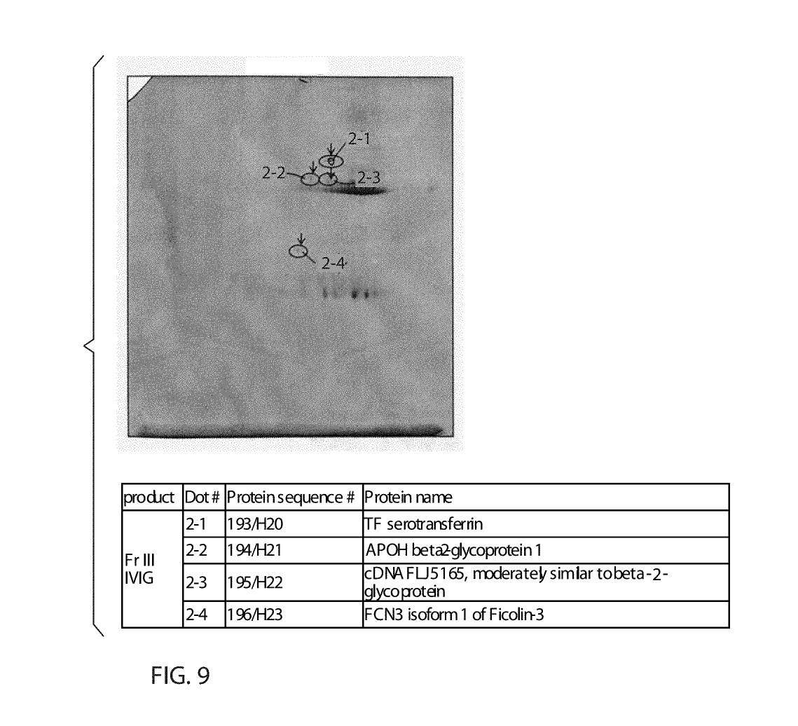

FIG. 9

4) AFOD RAAS 104 g. contains HEPATITIS B IMMUNEGLOBULIN with high titer of Hepatitis B antibody, in addition it contains TF protein sequence#197/H24 TF serotransferrin and may contain newly discovered proteins KH33, KH34, KH35, KH36 and KH37. The Hepatitis B antibody has been known to prevent the infection of the Hepatitis B virus in the health care worker, who may accidentally stick the contaminated needle from the Hepatitis B patient. In the product HepaRAAS® Hepatitis B immunoglobulin used to prevent the reoccurrence of the Hepatitis B virus in the liver transplant patient. In addition with the combination of one or many of these newly discovered proteins KH33, KH34, KH35, KH36 and KH37 the AFOD RAAS 104 can immediately stop the replication of the Hepatitis B virus in mice models and completely transform the Hepatitis B virus cell, which produces the sick protein that causes the Hepatitis B, into a good protein to eliminate the Hepatitis B virus in the mice within 4 days of 1 dose a day administration.

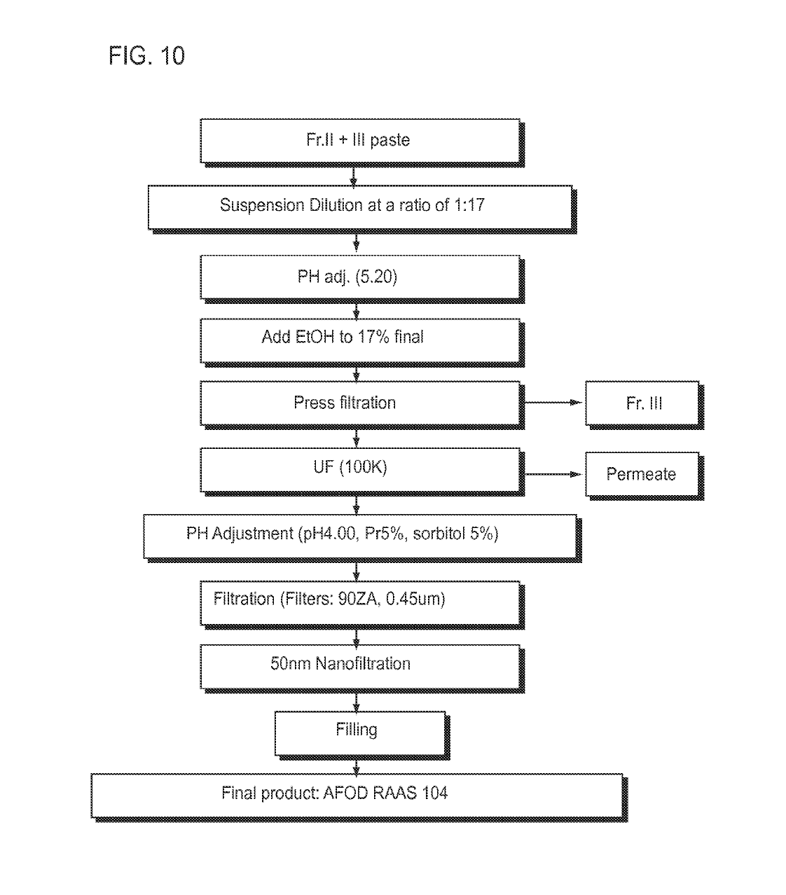

FIG. 10

Beside the main component of the Immunoglobulin in each of the three processes namely AFOD RAAS 102, AFOD RAAS 103 and AFOD RAAS 104 each product also has an additional proteins that differ from one another.

FIG. 11, 12.

Finally in the AFOD RAAS 102. we found the following proteins: IGHV4-31.; IGHG: 1. 44 kDa protein, IGHV4-31; IGHC1 32. kDa protein, IGHV4-31; 1GHG1. Putative uncharacterized protein DKFZp686G11190.

In AFOD RAAS 103 we found the following proteins: TF serotransferrin, APOH beta2-glycoprotein 1, eDNA FU5165, moderately similar to beta-2-glycoprotein, FCN3 isoform 1 of Ficolin-3.

In AFOD RAAS 104 we found the following protein: TF serotransferrin.

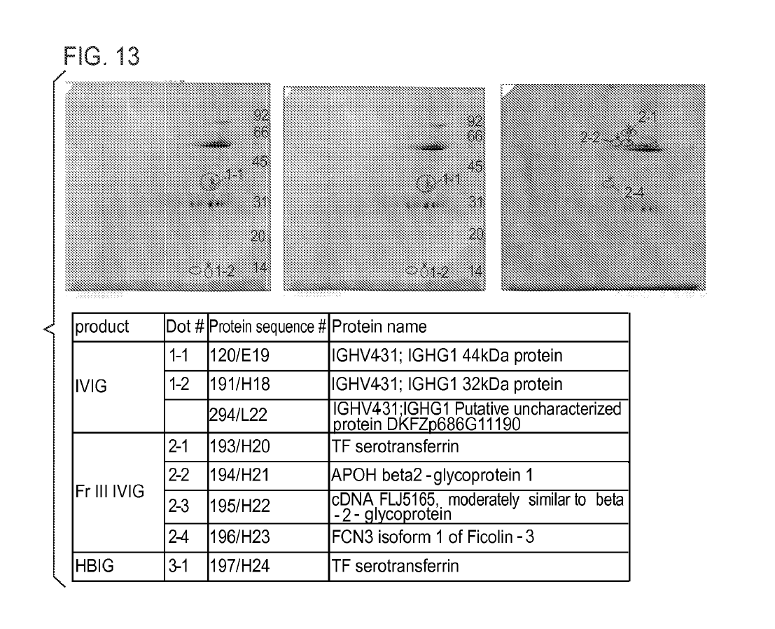

FIG. 13

5) AFOD RAAS 105® is formulated due to the scarcity of Hepatitis B antibody while the treatment for the Hepatitis B virus demands more of the product. AFOD RAAS 105 is the combination of

80% AFOD RAAS 102 and 20% AFOD RAAS 104. Both when combined will give more products

not only for Hepatitis B but also for the treatment of cancers, especially liver cancers or liver diseases, and other neurological diseases. Both of the products must have a concentration by ultra filtration up to 30%. This combination will provide the product of AFOD RAAS 105 with five newly discovered proteins KH33, KH34, KH35, KH36, KH37 and KH51 which may contain newly discovered GOOD HEALTHY CELLS which synthesize the new good proteins.

There are two methods of manufacturing AFOD RAAS 105®:

Method 1: Follow manufacturing protocol to separately manufacture normal Immunoglobulin and Hepatitis B antibody until the step of non-sterile final bulk for both products come, take 80% of the normal Immunoglobulin non-sterile final bulk and mix with

20% of Hepatitis B antibody non-sterile final bulk. Perform sterile filtration for filling for AFOD RAAS 105®

Method 2: Take 80% of normal immunoglobulin fraction II+III and 20% of Hepatitis B antibody fraction II+III then dissolve together in the process tank for production of the normal Immunoglobulin until the filling for AFOD RAAS 105@.

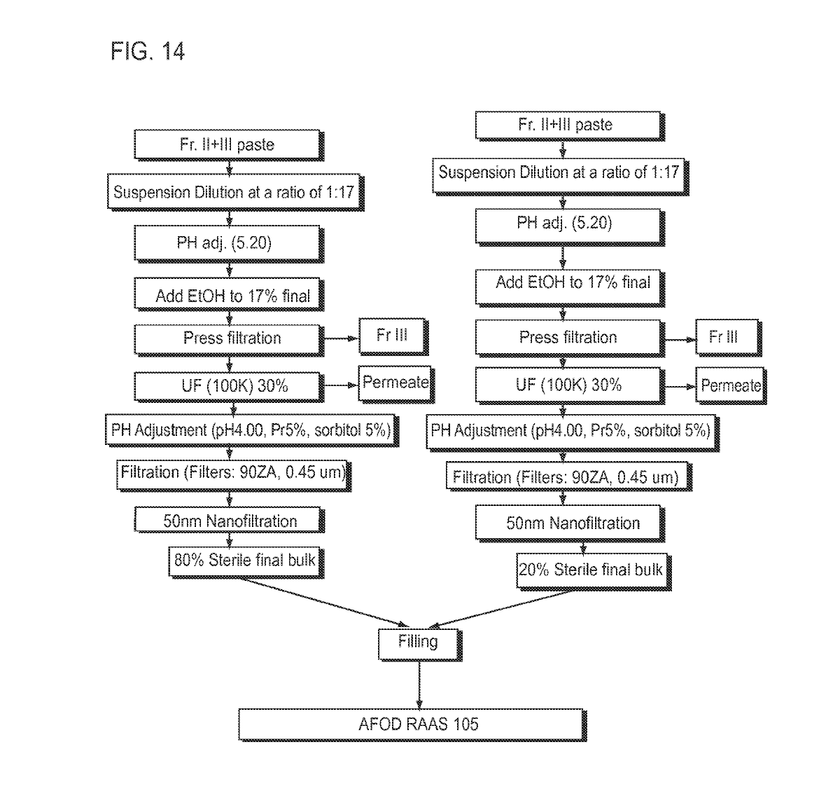

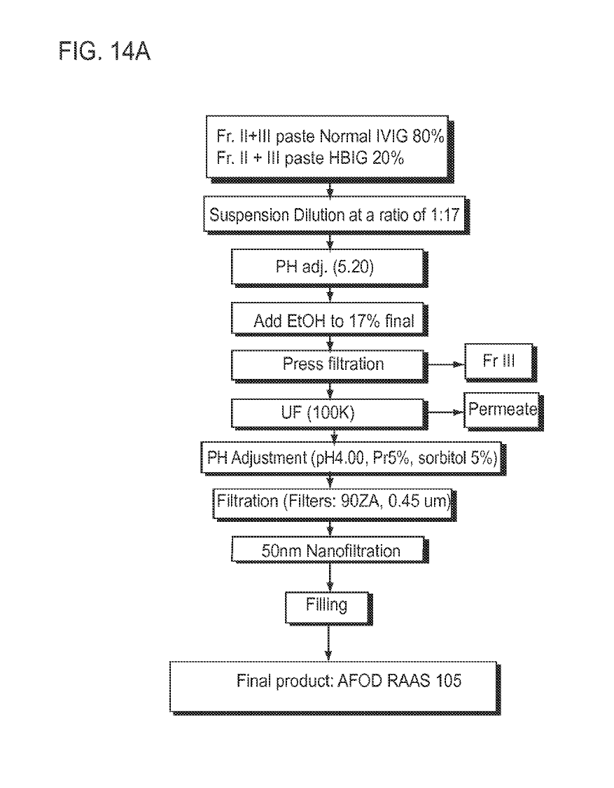

FIG. 14, 14a

6) AFOD RAAS 106@ is the combination of AFOD RAAS 101 with seven discovered proteins plus newly discovered KH51 and i\FOD RAAS 102 with a total of 8 proteins, including newly discovered protein KH33, KH34, Kh35, KH36 and KH37 has become a very potent combination of all this newly discovered proteins in Human Albumin and Immunoglobulin which enables this combination to work effectively against all cancers, bacteria, specially staphylococcus aureus which is resistant to the current antibiotics.

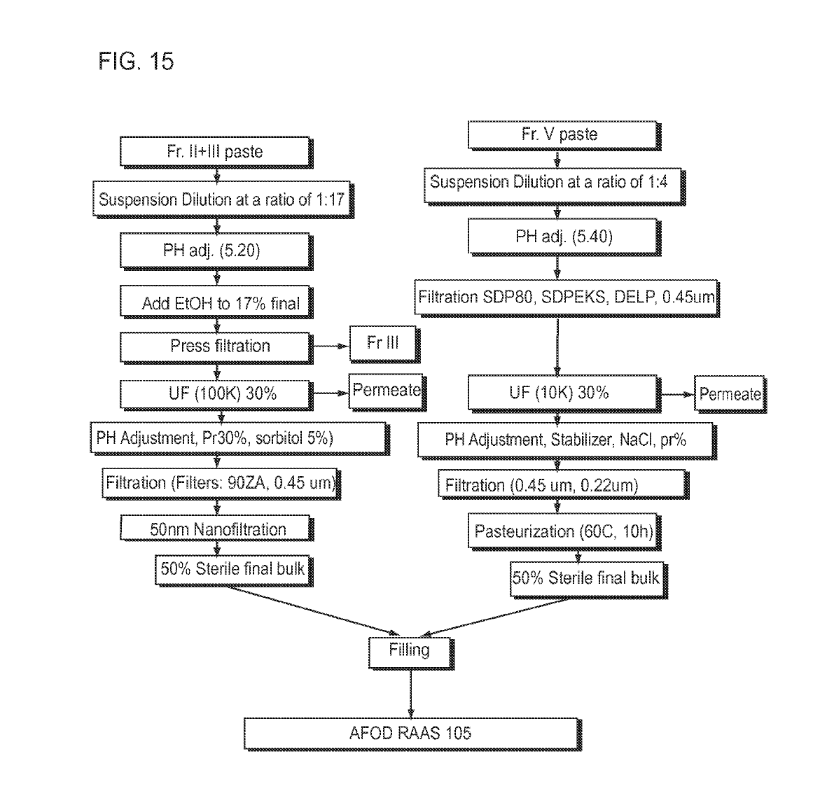

FIG. 15

7) AFOD RAAS 107® contains mainly the protein 1CP 98 kDa and possibly a lot more new proteins that are under investigation. Protein 1CP 98 kDa contain Nup98 and Nup96 play a role in the bidirectional transport. The 98 KD nucleoporin is generated through a biogenesis pathway that involves synthesis and proteolytic cleavage of a 186 KD precursor protein. The human gene has been shown to fuse to several genes following chromosome translocations in acute myelogenous leukemia (AML) and T-cell acute lymphocytic leukemia (T-ALL). This gene is of the several genes located in the imprinted gene domain of 11p15.5, an important tumor-suppressor gene region. Alterations in this region have been associated with the Beckwith-Wiedemann syndrome, Wilms tumor, rhabdomyosarcoma, adrenocortical carcinoma, and lung, ovarian and breast cancer.

This protein along with a lot more new proteins under investigation have proven efficacy against the breast cancer and other cancers as described above.

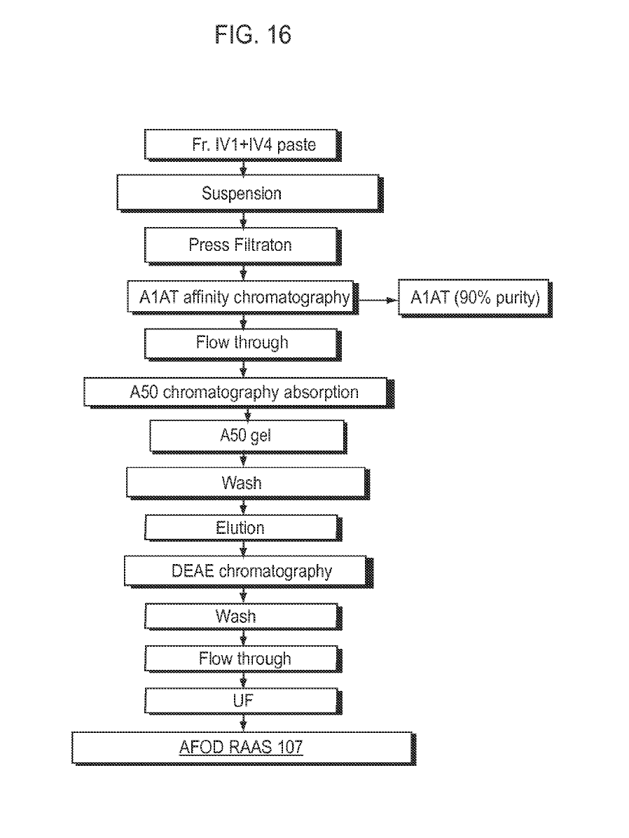

FIG. 16

20 electropherosis of plasma derived protein CP98 kOa shows numerous newly discovered KH proteins, more new proteins under investigation or already discovered proteins.

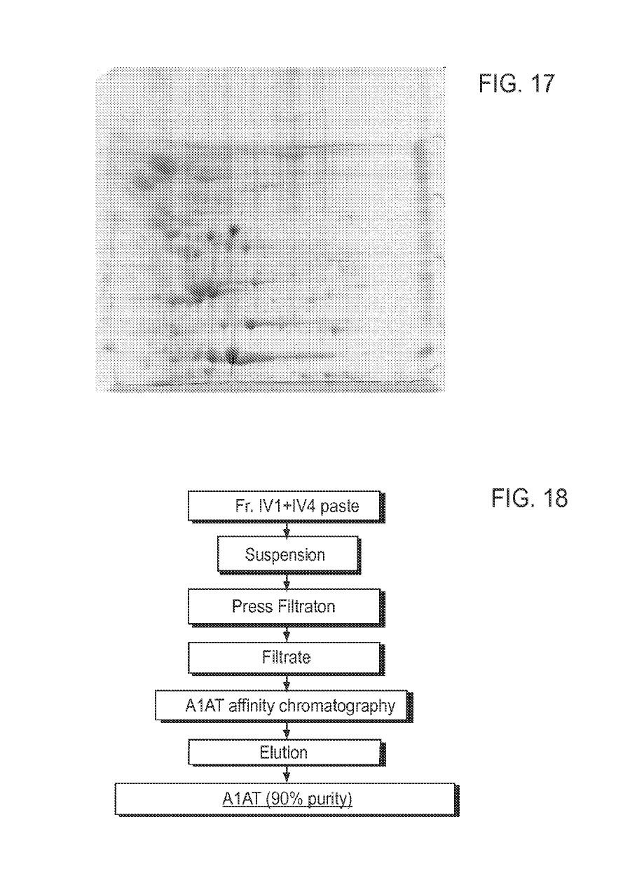

FIG. 17

8) AFOO RAAS 108 g. contains mainly Alpha 1 antitrypsin protein which has been used in the treatment of the Alpha 1 Antitrypsin deficiency and also for the treatment of emphysema. Currently it is also being used under trial for Diabetic patients. With the complex of the new found proteins like KH21, KH22, KH23, KH24, KH25, KH26, KH27, KH48, KH49 and KH50 the efficacy of AFOD RAAS 108 will be more effective in the treatment of cancers, diabetic and many other diseases or deficiencies.

FIG. 18

20 electropherosis of plasma derived protein A1AT shows numerous newly discovered KH proteins, more new proteins under investigation or already discovered proteins.

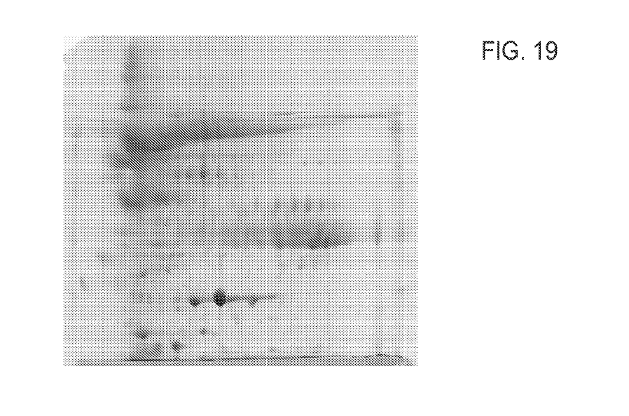

FIG. 19

9) AFOO RAAS 109® contains mainly Transferrin protein which has not been used for any clinical application however used for diagnostic purpose. With the complex of the new found proteins like KH2J, KH2.2, KH2.3, KH2.4, KH25, KH26, KH27, KH48, KH49 and KH50 the efficacy of AFOD RAAS 109 will be more effective in the treatment of cancers, diabetic, cardiovascular and many other diseases or deficiencies. The inventor believes that with enough dosage of AFOD RAAS

109 it will provide enough good healthy cells to synthesize the protein which produces insulin in the patient to certain point that the patient will no longer need to inject the insulin anymore.

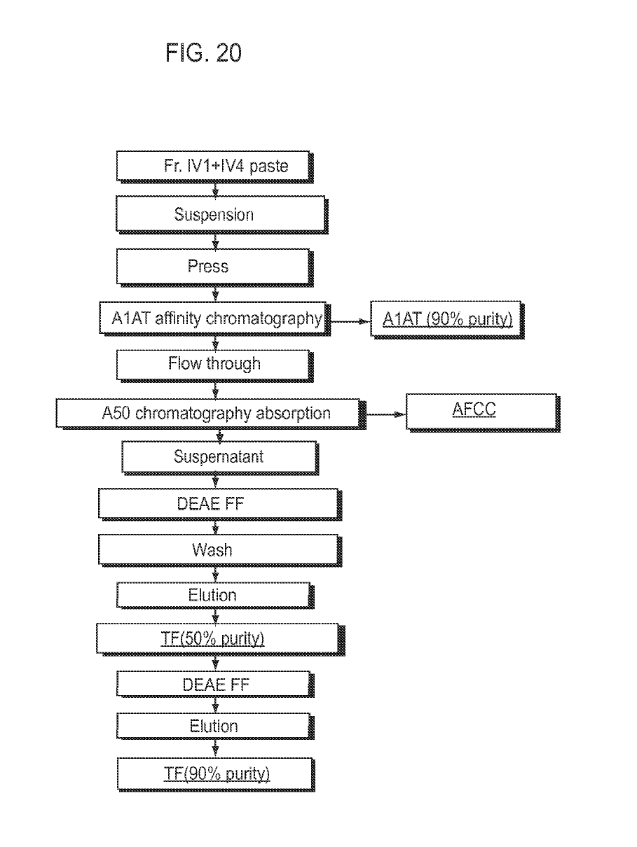

FIG. 20

20 electropherosis of plasma derived protein Transferrin shows numerous newly discovered KH proteins, more new proteins under investigation or already discovered proteins.

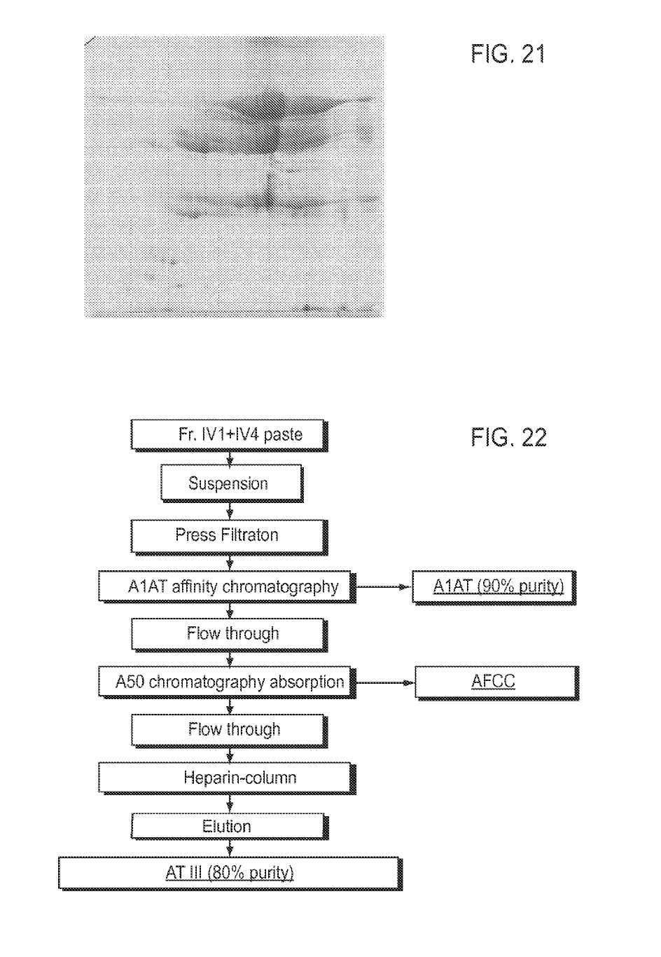

FIG. 21

10) AFOD RAAS 110 g. contains mainly AntiThrombin III protein commercially available but with no significant efficacy has been proven. With the complex of the new found proteins like KH21, KH22, KH23, KH24, KH25, KH26, KH27, KH48, KH49 and KH50 the efficacy of AFOD RAAS 110 will

be more effective in the treatment of thrombosis, stroke patients and cardia vascular diseases in combination with AFOD RAAS 1(APOA1)

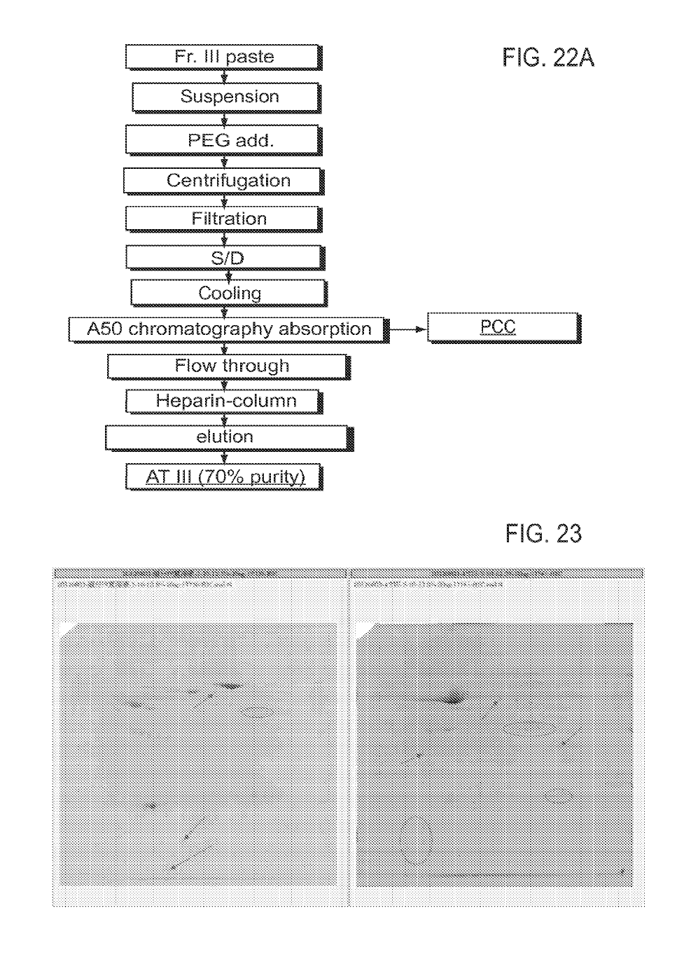

FIG. 22, 22a

11) AFOD RAAS 111 g. mainly beside Human Albumin, it also contains newly discovered proteins like

KH21, KH22, KH23, KH24, KH25, KH26, KH27, KH48, KH49 and KH50. The efficacy of AFOD RAAS

111\NilI be more effective. The inventor believes that with enough dosage of AFOD RAAS 111 it will provide enough good healthy cells to synthesize the protein which produces insulin in the patient to certain point that the patient will no longer need to inject the insulin anymore.

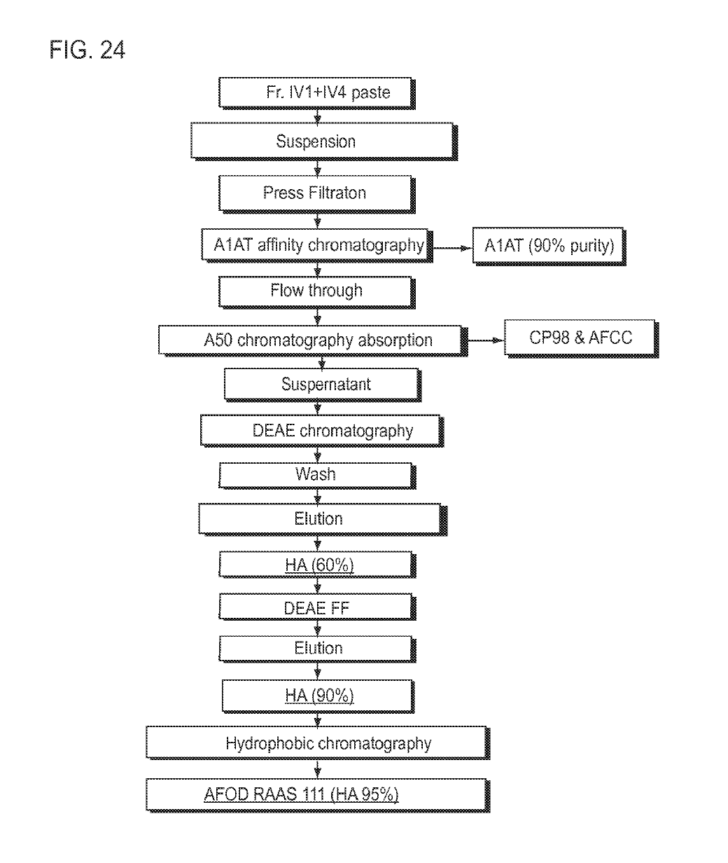

FIG. 24

12) AFOD RAAS 112® contains a small amount of the Human Albumin protein, however this Human Albumin together with the newly discovered protein KH3, KH4, KH5, KH6, KH7, KH8, KH9, KI-UO, KH19, KH20, KH38. KH39, KH40, KH41, KH42 and KH43 have been known through our animal studies, to prevent the death caused by H1N1 virus in the mice. It also has shown in vitro studies to eliminate the HIV virus. rv1ore proteins from AFOD RAAS 112 are under investigation. The inventor believes that with enough dosage of AFOD RAAS 112 it will provide enough good healthy cells to synthesize the protein which produces insulin in the patient to certain point that the patient will no longer need to inject the insulin anymore.

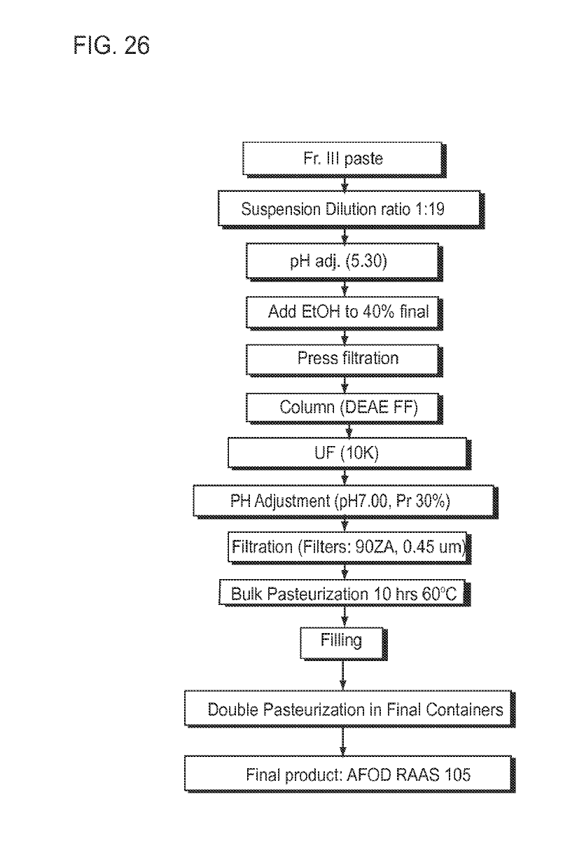

FIG. 26

:1.3) AFCC RAAS 101® contains mainly protein Human Coagulation Factor VIII mainly for use in the stop of the bleeding in patients with Hemophilia A. However AFCC RAAS 101 not only contains Coagulant Factor VIII but it also contains newly discovered proteins KH1, KH2, KH2.8 and KH29. With the addition of these newly found proteins which has shown in in-vitro studies to reduce the tumor growth of solid cancers. The inventor believes that with enough dosage of AFCC RAAS

101 it will provide enough good healthy cells to synthesize the Factor VIII protein in the patient to certain point that the patient will no longer need to inject coagulant factor VIII anymore.

FIG. 2.8

20 electropherosis of plasma derived protein Human Coagulation Factor VIII shows numerous newly discovered KH proteins, more new proteins under investigation or already discovered proteins.

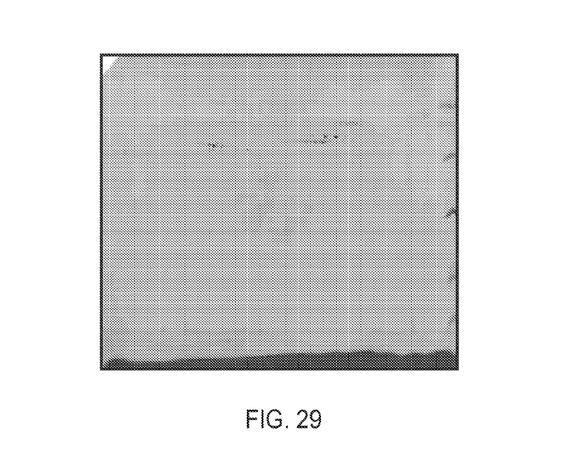

FIG. 29

14) AFCC RAAS 102® contains mainly Human Fibrinogen protein which is used mainly for the treatment of liver diseases and trauma. With the addition with our five newly discovered proteins KH1, KH2, KH30, KH31 and KH32 has shown in in-vitro studies to reduce the growth of solid tumors.

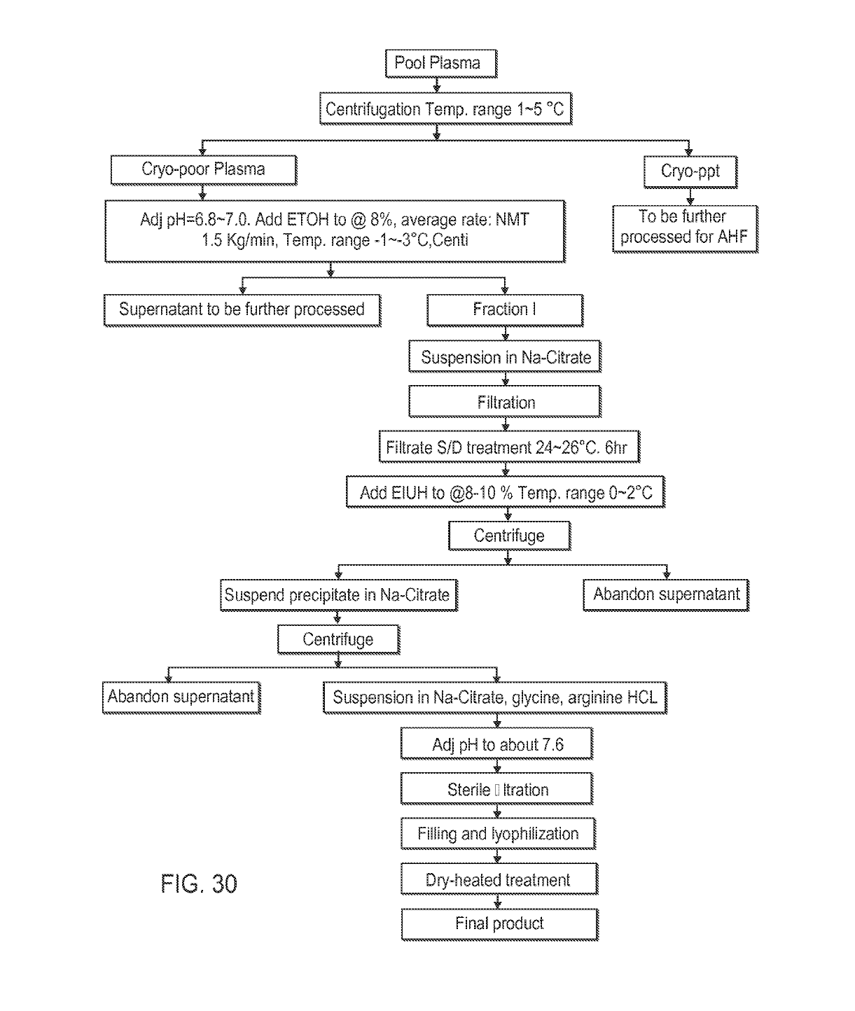

FIG. 30

20 electropherosis of plasma derived protein Human Fibrinogen shows numerous newly discovered KH

proteins, more new proteins under investigation or already discovered proteins.

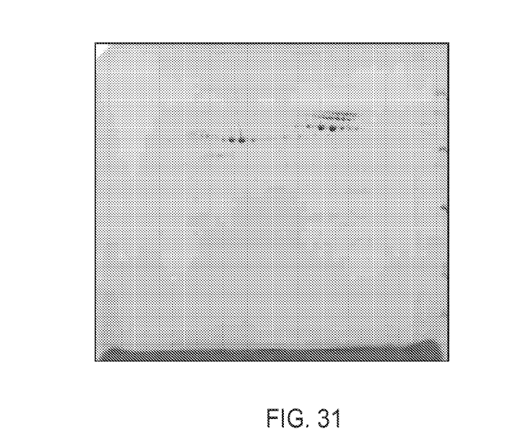

FIG. 31

15) AFCC RAAS 103® contains mainly High Concentrate Human Fibrinogen protein which is used in combination with Thrombin to create a Fibrin Glue membrane (as in FibringluRAAS®) in order to stop the bleeding during the surgical operations. With the addition of newly discovered proteins KH1, KH2, KH30, KH31, KH32 and specially KH52 AFCC RAAS 103® has been proven to be very effective in stopping the tumor growth in liver cancer, colon cancer and lung cancers in animal studies which are used for the submission of the application for licensing.

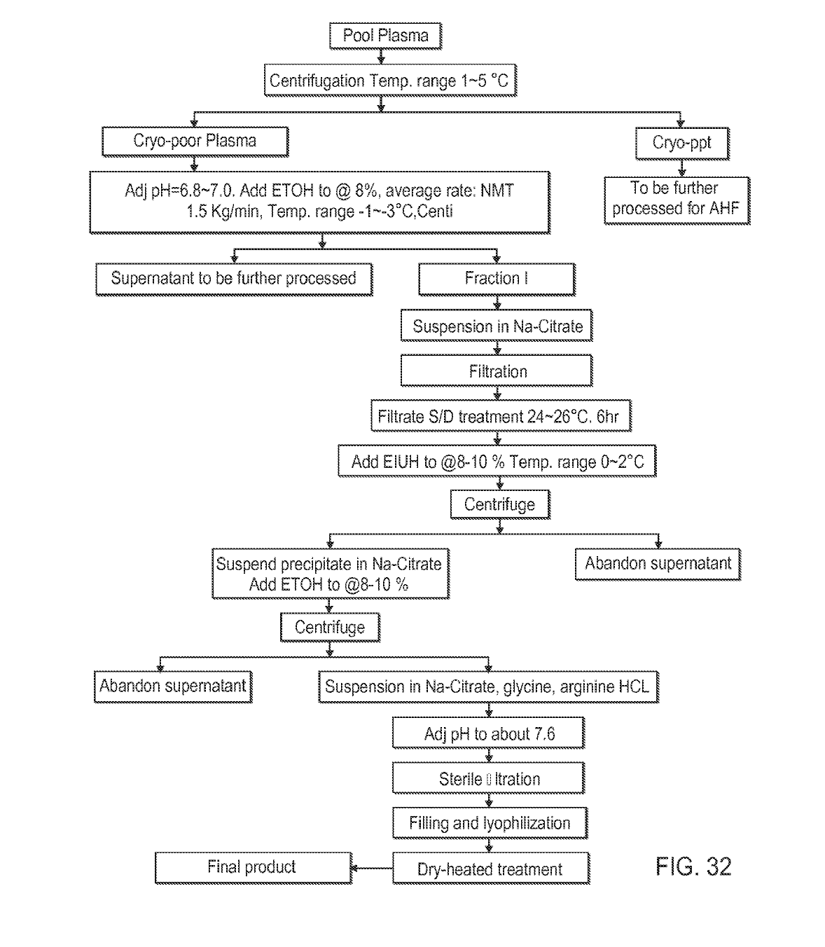

FIG. 32.

20 electropherosis of plasma derived protein High Concentrate Human Fibrinogen shows numerous newly discovered KH proteins, more new proteins under investigation or already discovered proteins.

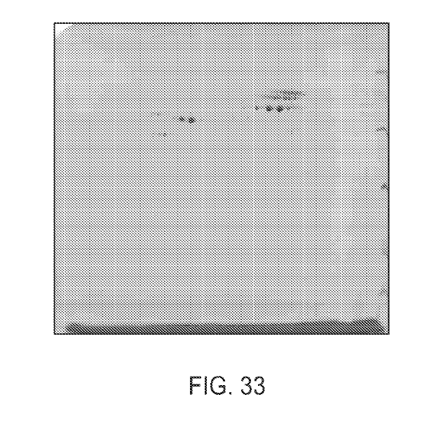

FIG. 33

16) AFCC RAAS 104® contains mainly Human Thrombin protein which is used in combination with High concentrate Human Fibrinogen protein to create a Fibrin Glue membrane (as in FibringluRAAS®) in order to stop the bleeding during the surgical operations. With the addition of newly discovered proteins KH44, KH45, KH46 and KH47 in our AFCC RAAS 104® has been proven to be very effective in stopping the tumor growth in liver cancer. colon cancer and lung cancers in animal studies which are used for the submission of the application for licensing.

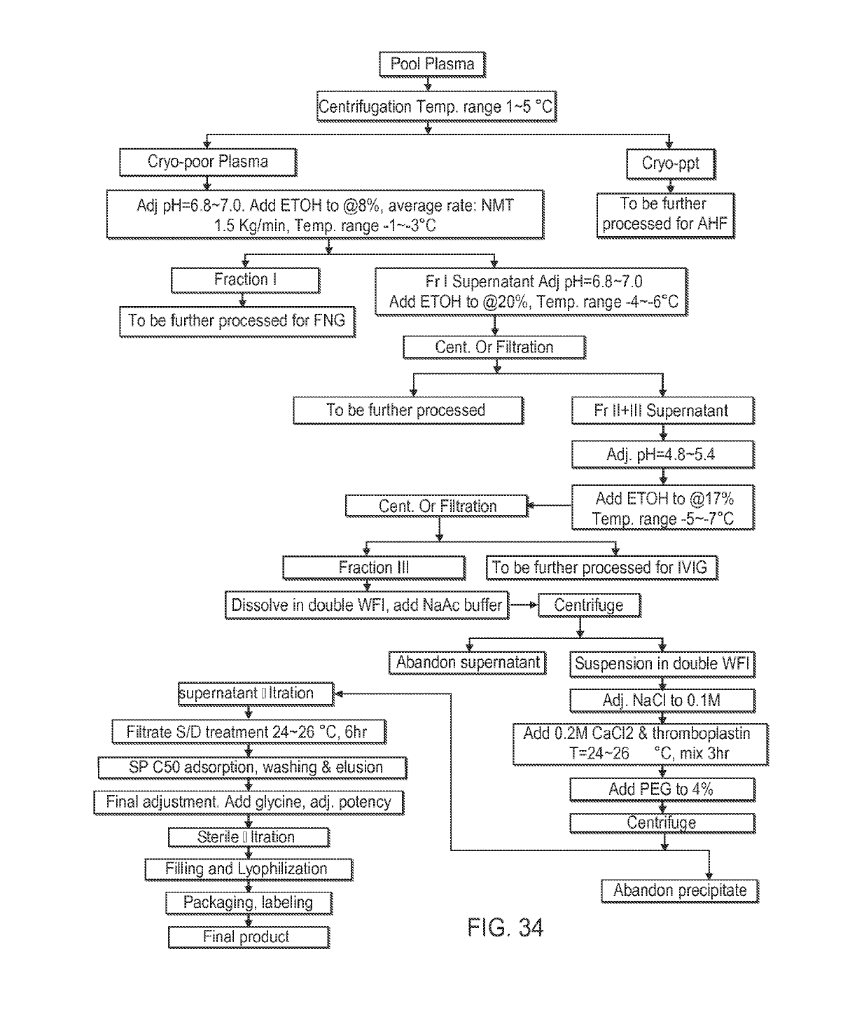

FIG. 34

2D electropherosis of plasma derived protein Human Thrombin shows numerous newly discovered KH

proteins, more new proteins under investigation or already discovered proteins.

FIG. 3.5

17) AFCC RAAS 105® contains mainly Human Prothrombin Complex protein which include Factor II, Factor VII, Factor IX and Factor X. In the world it is mainly used for the treatment of Hemophilia Bas a Factor IX or it can be used for Hemophilia A treatment with inhibitor. In China Prothrombin Complex is used mainly in the treatment of the liver disease. AFCC RAAS 105@ contains eight newly discovered proteins: Kf-111, Kf-112, KHB, Kf-114, KH15, KH16, KH17 and

KH18. The inventor has found that the HIV virus cannot be killed in PCC by solvent detergent method using TNBP and TWIN80, that led to the in-vitro testing of the original AFCC RAAS 105 (formerly AFCC RAAS 1) and has found that the HIV virus has been eliminated in enzyme also the viral load has become negative in the PCR testing. Confirmation of the HIV replication and the animal study is being done with the help of the National AIDS research center at Tsing Hua University in Beijing. This formulation can only be used for the Hemophilia A or B with HIV, but

for non hemophilia patients the dosage and prescription must be highly controlled from the physician, because if too much product is given then the patients could be fatal.

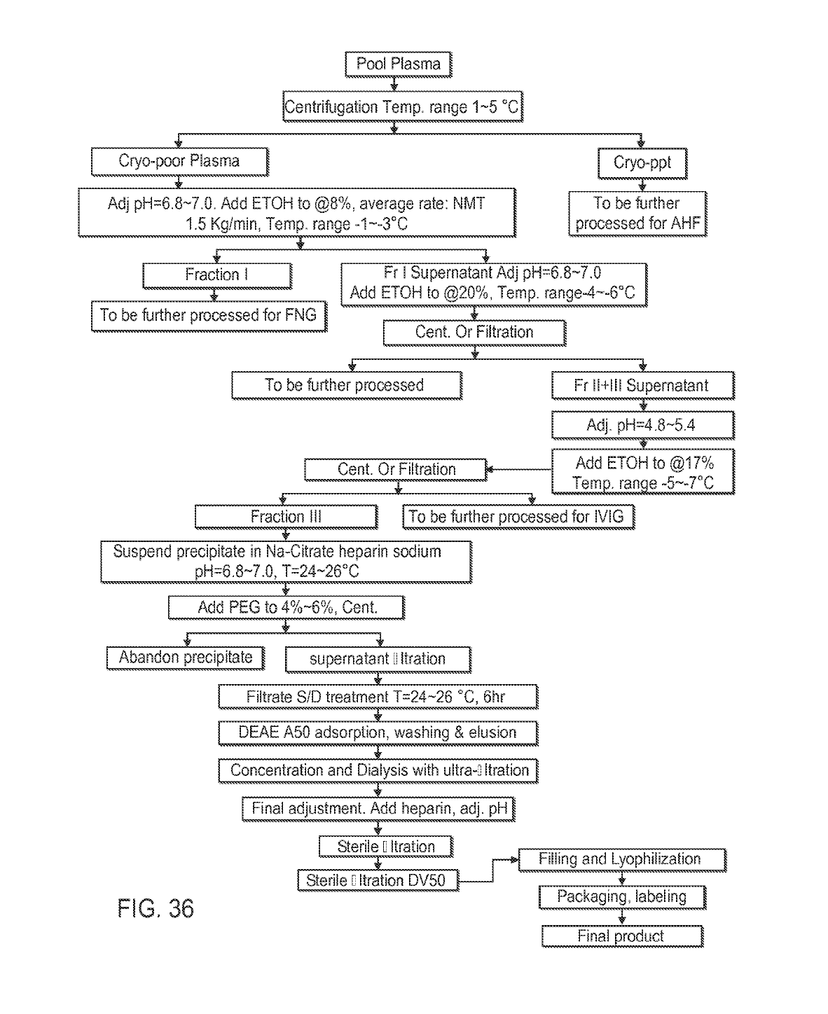

FIG. 36

2D electropherosis of plasma derived protein Human Prothrombin Complex shows numerous newly discovered KH proteins. more new proteins under investigation or already discovered proteins.

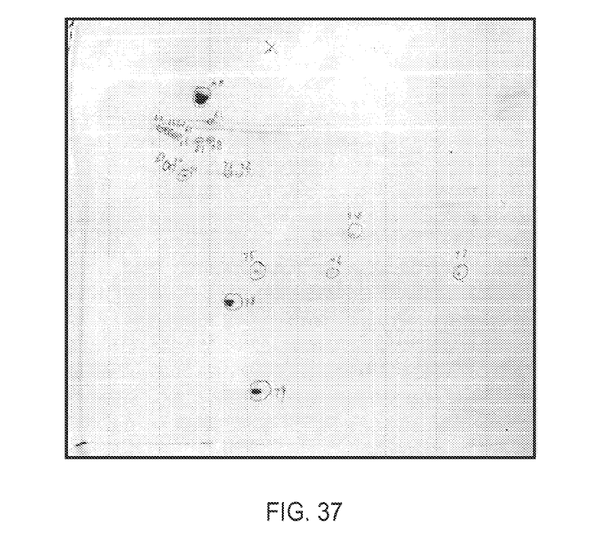

FIG. 37

:1.8) AFCC RAAS 106® mainly contains all newly discovered proteins KH2J, KH2.2, KH2.3, KH2.4, KH25, KH26, KH27, KH48, KH49 and KH.SO in fraction IV. The color of which is blue from pile, so we assume that it is PCC. But when we tested for the content of Factor IX, we were not able to find any factor IX. The Inventor see the problem associated with AFCC RAAS 10.5® as they are from fraction III and this is the most complicated complex of proteins which include Prothrombin and Thrombin therefore the inventor wants to have the same product of AFCC RAAS: 1.05® which can kill the HIV virus or others but will not cause harm to the NON hemophilia patients, therefore

this formulation was created.

2D electrophoresis of plasma derived proteins in i\FCC from fraction IV in the red circles and red arrows shows numerous newly discovered KH proteins, more new proteins under investigation or already discovered proteins.

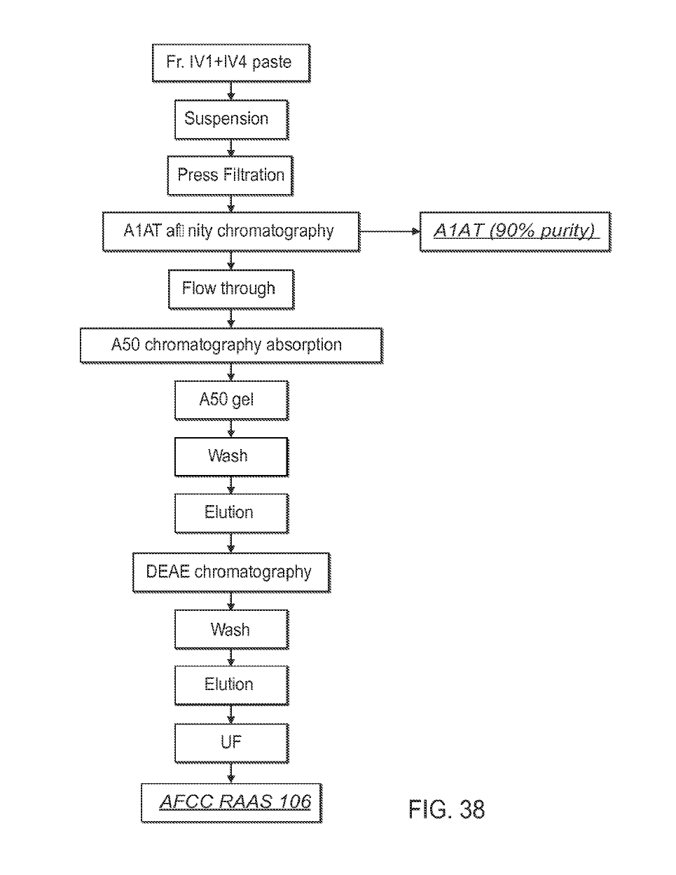

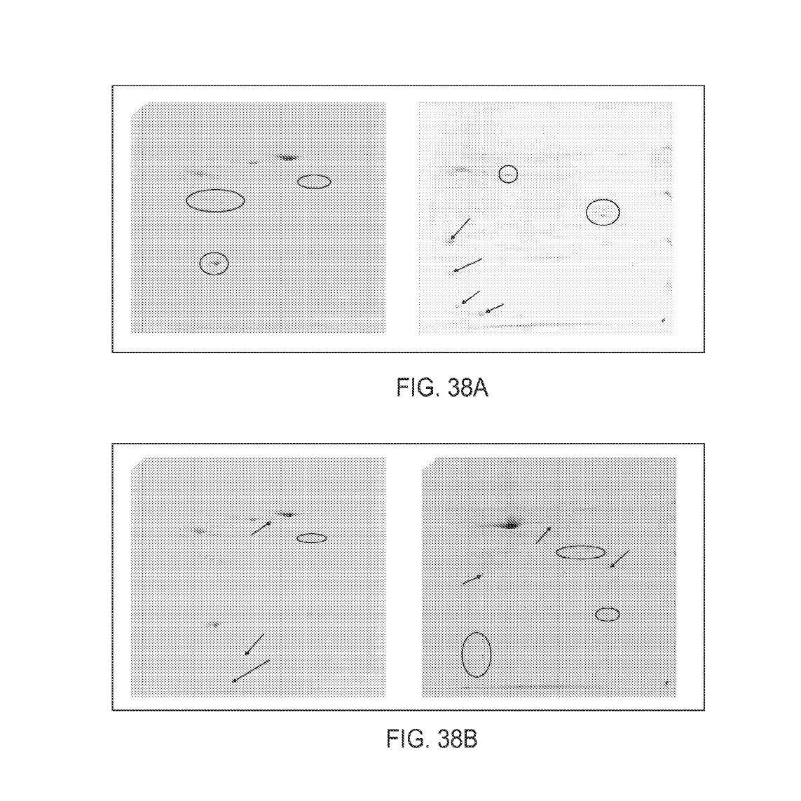

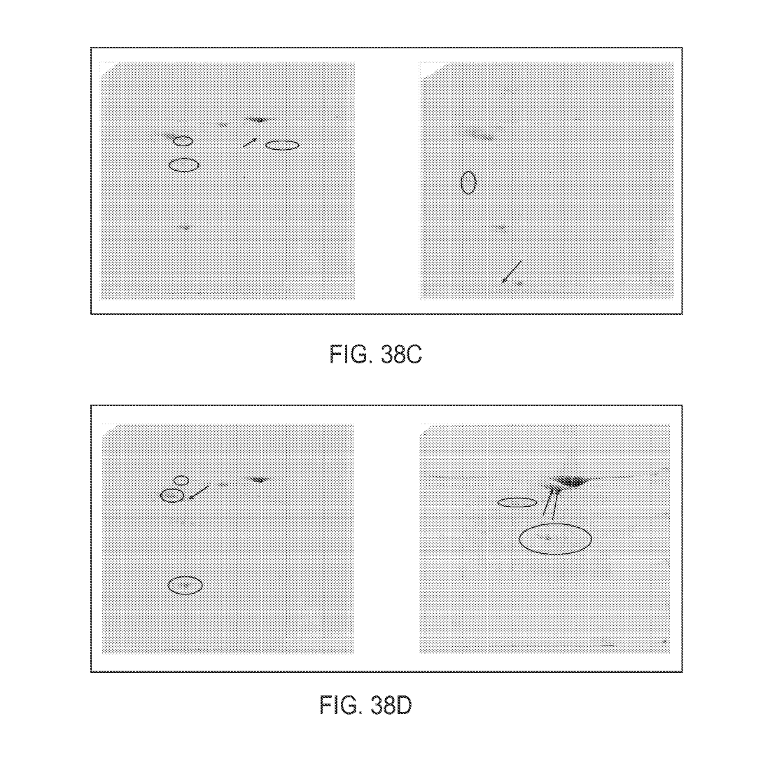

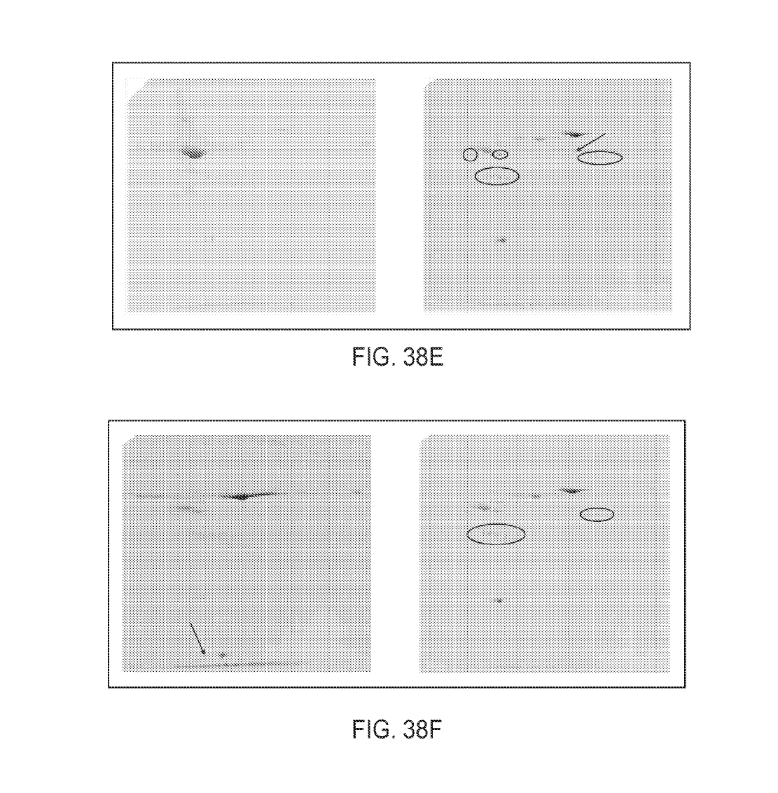

FIG. 38a

20 electrophoresis of plasma derived protein Anti Thrombin III from fraction IV in the red circles and red arrows shows numerous newly discovered KH proteins, more new proteins under investigation or already discovered proteins.

FIG. 38b

2D electrophoresis of plasma derived protein CP98 from fraction IV in the red circles and red arrows shmNs numerous newly discovered KH proteins, more new proteins under investigation or already discovered proteins.

FIG. 38c

2D electrophoresis of plasma derived protein Transferrin from fraction IV in the red circles and red arrows shows numerous newly discovered KH proteins, more new proteins under investigation or already discovered proteins.

FIG. 38d

20 electrophoresis of plasma derived protein Alpha 1 Antitrypsin from fraction IV in the red circles and red arrows shows numerous newly discovered KH proteins, more new proteins under investigation or already discovered proteins.

FIG. 38e

2D electrophoresis of plasma derived containing only pure protein Alpha 1 Antitrypsin from fraction IV.

FIG. 38f

ANIMAL Blood Plasma

In the animal study we have found the prevention of influenza H1N1 which can also affect the birds, therefore the inventor has discovered using the same process of AFOO RAAS 101 through AFOO RAAS

also utilized in the blood plasma of healthy animals to fractionate and further process into the product like Human Albumin and immunoglobulin, and others for the prevention of the infection of the virus like H1N1, SARS, H5N1, foot and mouth disease, mad cow disease and other epidemic unknown diseases.

FDA has recently forbidden the use of antibiotic in the cow as the antibiotic are resistant and It could get to the population.

In our study of the H1N1 for the prevention of the H1N1 virus after one week of injection, the mice has survived as the product has injected the good healthy cells that send the signal to the DNA to transform the RNA of these infected mice to produce a good protein against the H1N1 virus. The long term study

of how long this protection will last is still ongoing, so far the study has been going for 6 weeks. H1N1 is not as so important as the foot, hand and mouth disease that affects over 1 million people in China right now.

In addition to that we can test for mad cow disease but so far we have neither vaccine, nor product to take care of mad cow disease which has caused England not to allow their population to donate plasma and to import plasma from the United States of America.

In the USA we randomly check the cows and recently it was discovered some cases of mad cow disease. In Vietnam there are cases of Pigs with blue ear disease and in China H5N1 influenza has been found.

In brief there are still a lot of animals that are in as much danger as the human being for the virus infections and at any moment there could be an outbreak, if the animals are not vaccinated or treated with these products.

These products are not only for the prevention but to cure the diseases and to stop the disease from spreading, therefore meat eaters can feel safe about consuming any type of meat, since there is no use of hormones, antibiotic or chemical drugs in their bodies that can affect the consumer health.

AHC: RAAS 1 through AHC: RAAS 10 are under development to cure or prevent the any disease or outbreak in cows, pigs, chicken, lamb, goat. sheep.

This product can also prevent the death of animals such as Panda. When they are sick and there is no product to protect and treat them. Also the strongest and fierce animal such as the Tiger could be saved as in the incident in October 2004 in Thailand, the inventor has found that ninety tigers from Thai Zoo had died after eating the carcass of the bird flu chicken.

The investigation is undergoing for different kind of animals and of course we will discover more cells and proteins, like the case in human that we are doing.

With the good healthy cells of any animal to send the signal to the DNA to transform the RNA in order to synthesize the good healthy proteins to fight the disease and infections in any animal.

Recombinant DNA Proteins

Due to the shortage of plasma worldwide for the production of plasma derived products we have come up with also recombinant DNA proteins using the existing sequences of those existing proteins and specially the inventor has discovered 52 newly found proteins with their sequences and he has come up with different process following the process of making recombinant factor VIII. The plasmid construction for both mammalian yeast has been constructed, following the sequence of our newly found 52 proteins KH1, KH2, KH3, KH4. KH5, KH6, KH7, KH8, KH9, KH10, KH11, KH12, KH13, Kf-114,

KH15, KH1KH17, KH1KH1KH2KH2L KH2KH23, KH2KH25, KH26, KH27, KH28, KH2 KH30, KH31, KH32, KH33, KH34, KH35. KH36, KH37, Kf-138, Kf-139, Kf-140, Kf-141, Kf-142, KH43, KH44, KH45, KH46, Kf-147, Kf-148, Kf-149, KHSO, KH51 and Kf-152.

In addition to this new found proteins we have created a recombinant factor VIII which contain this new sequences. This recombinant factor VIII, factor VII or Von Willebrand can cure the Hemophilia patient with Hepatitis B, Hepatitis C, HIV and eventually build enough coagulant for the Hemophilia

A or Hemophilia B.

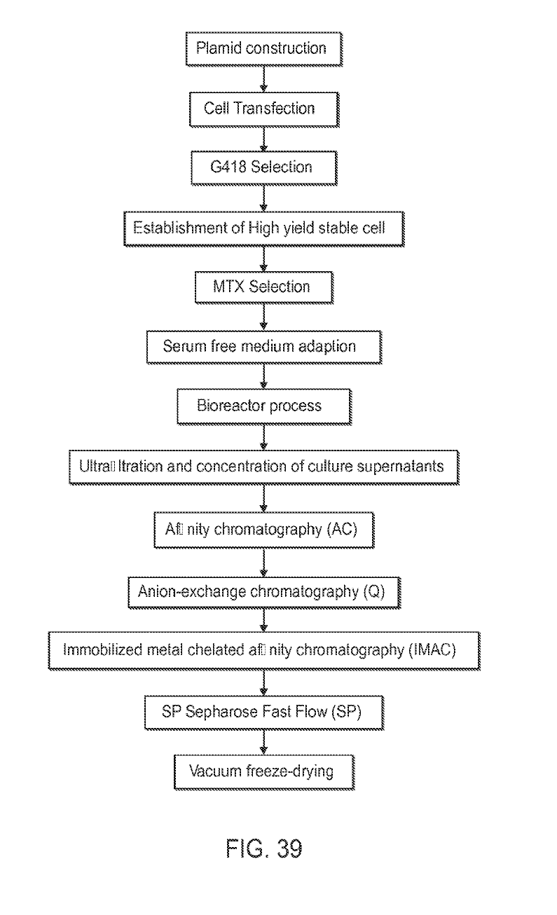

FIG. 39

Monoclonal Antibodies

In certain products like Hepatitis B antibody AFOD RAAS 104® with the new found proteins KH made from the high titer Hepatitis antibody from the human healthy donor are very short in supply. Monoclonal Antibodies can be created for such a major product, as they can cure Hepatitis B virus and liver cancer or any disease that is associated with the liver. In addition to this Hepatitis B monoclonal antibody. the plasmid construction of the following sequences of our newly found 52 proteins KH1, KH2, KH3, KH4, KHS, KH6, KH7, KH8, KH9, K1•110, KH11, KH12, KH13, KH14, KH15,

KH1KH1KH1KH19, KH2KH21, KH22, KH23, KH2KH25, KH2KH2KH28, KH2KH3 KH31, KH32, KH33, KH34, KH35, KH36, KH37, KH38, KH39, KH40, KH41, KH42, KH43, KH44, KH45, KH46, KH47, KH48, KH49, KH50, KH51 and KH52 to make the monoclonal antibodies with good proteins synthesized by the good healthy cells.

To cure diseases, viruses infections, bacteria infections, auto immune disease, neurological disorder, all type of solid and blood cancer, coagulation, diabetic, inhibitor, immune deficiency, muscle and nerve repair and restoration from Human or animal.

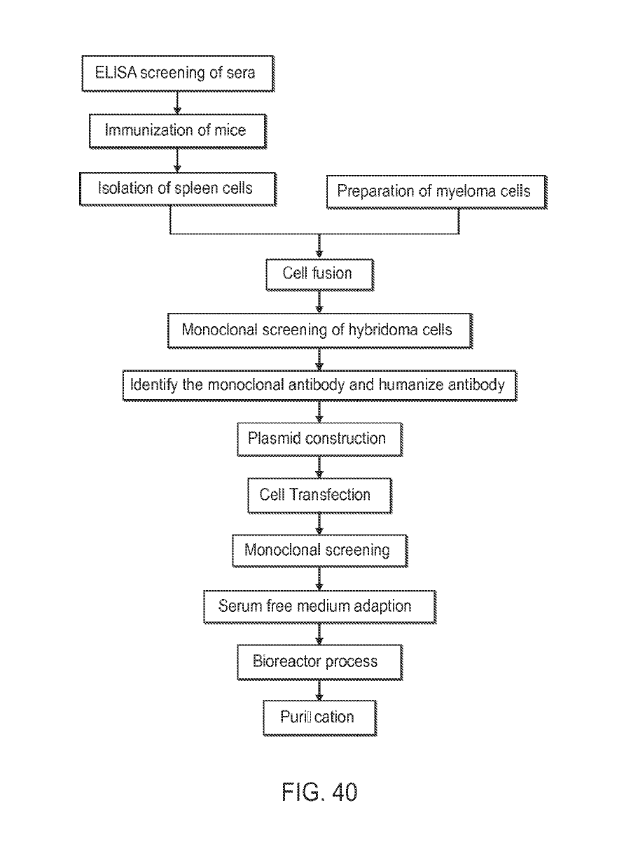

FIG. 40

The use of cultured cell from a product to express in order to obtain the desired proteins. The inventor has discovered a number of new cells under different patent. The discovery led to the use of existing products like AlbuRAAS®, GammaRAAS®, HemoRAAS®, ProthoRAAS®, FibroRAAS®, ThrombiRAAS®, FibringluRAAS® and HepaRAAS® to culture to obtain the desired cell for expression, in addition to the newly discovered cells.

The desired cells can be obtained through culture of the plasma or the fraction or the final products including the AFOD RAAS and AFCC RAAS products.

After harvesting the desired cells for a certain protein, the cell expression to increase the cell population to produce enough desired proteins for further process in the final product.

Such a method include the selection of various mediums or amino acids to help grow the cells.

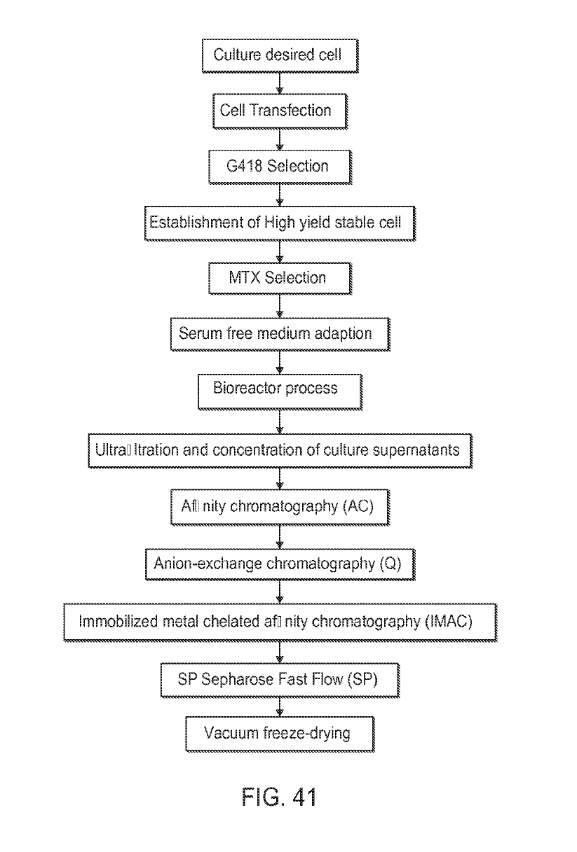

FIG. 41

The manufacture of AFOD RAAS and AFCC RAAS products by using the direct cell from cell culture for expression to synthesize the desired already discovered or newly found proteins.

In this study we also found a lot of cells from different mediums of plants, fruits, vegetables, rice, Oatmeal or any source of meat or seafood, it was amazing that we have found a lot of cells in these mediums which can generate the cells within seconds to get up to 20-30 million cells, while the CHO cell for our recombinant factor VIII it will take a week to grow up to 10 million cells.

We also use 50 g of rice to produce 5 liters of medium and instantly this medium has 2.0 million cells, using this medium to mix with our products of Human Albumin and Immunoglobulin to observe the growth of cells for expression.

The same process can apply for the existing products as stated above and the newly discovered proteins KH1, KH2, KH3, KH4, KH5, KH6, KH7, KH8, Kf-19, K1-110, KH11, KH12, KH13. KH14, KH15, KH16, KH17, KH1KH19, KH2KH2L KH22, KH2.3, KH2, KH2.5, KH2.6, KH2.7, KH28, KH29, KH3

Kf-131, KH32, KH33, KH34, KH35, KH36, KH37. KH38, KH39, KH40, Kf-141, Kf-142, Kf-143, KH44, KH45, KH46, KH47, KH48, KH49, Kf-150, Kf-151 and KH52.

Thrombin which contains good protein, synthesized by good healthy cells can be delivered by microscopy.

In order to have products for oral applications by metabolism the enzymes of all these products can be extracted formulated in powder form and put in a capsule.

In conclusion all these processes can provide all products for the following routes of applications

1. In liquid form for injection.

2. In powder form for topical applications

3. Enzyme in powder in capsule for oral application

Mechanism

KH 1-through KH-52, and more KH proteins are being discovered in GOOD HEALTHY CELLs—named KH CELLS. KH CELLS are GOOD HEALTHY CELLS in which the RNA synthesizes good proteins that:

1—Send signal to the DAMAGED, SICK, AND BAD CELLS that triggers that synthesis of good proteins that transform these cells to become GOOD healthy cells.

2—Send signal to the other currently undamaged cells to synthesis of good proteins to protect them from being DAMAGED, INFECTED and PRONE to DNA and other cellular alterations.

3—Send signal to the body to produce new cells that are healthy and forbid them from being affected by intra- and extracellular damaging signals.

The mechanisms that govern these processes is the KH good healthy cells provide innate good signals that make good proteins to boost the immune system in order to CURE, TO PROTECT, and TO PREVENT diseases, viruses infections, bacteria infections, auto immune disease, neurological disorder, all type of solid and blood cancer, coagulation, diabetic, inhibitor, immune deficiency, muscle and nerve repair and restoration from Human, animal or substances by the method of fractionation, purification, recombinant DNA, monoclonal antibody, transgenic and expression of cells from the cultured GOOD HEALTHY CELLS.

The following studies have been performed to provide critical evidence for the three mentioned above mechanisms:

1) The study of APOA1 protein in preventing atherosclerosis and related cardiovascular diseases

2) The lipid profile results and quantification of atherosclerosis plaque in 18 ApoE mice fix 4 weeks study.

3) RAAS AFOD RAAS 1(APOA1) in ApoE mice for 8 weeks.

4) RAAS AFOD RAAS 1(APOA1) in ApoE mice for 16 weeks.

5) Efficacy study of RAAS antibodies on Type 2 diabetic mouse model in db/db mice

6) In Vivo Efficacy Testing of eight RAAS compounds in 4T1-LUC Breast Cancer Cell Orthotopic Model

7) In Vivo Efficacy Testing of eight RAAS compounds in 4T1-LUC Breast Cancer Cell Orthotopic Model

8) Anti-tumor efficacy of high concentrated fibrinogen enriched alat thrombin and Afod (FS) in combination with Afod RAJ\.S 2 or Mod RA.AS 4 in patient-derived tumor xenograft (PDX) models in nude mice.

9) Characterization of lymphoid tissues and peripheral blood in nude mouse treated with and without AFCC.

10) Antiviral efficacy of AFOD RAAS-2 in an influenza H1N1-infected mouse model

11)″″″″″″ ″″>? of AFOD on 6-OHDA rat model of Parkinson's disease

12) Antiviral efficacy of AFCC in an influenza EI1N1-infected mouse model

13) Antiviral efficacy of AFOD and AFCC in an influenza H1N1-infected mouse model

14) Efficacy of AFOD RAAS 104C:8:) (formerly AFOD RAAS 8) in the EIBV Mouse Hydrodynamic Injection Model.

The recent tsunami and earthquake in Japan in March of 2011, caused panic and economy loss not only in Tokyo but around the world as people tried to escape from Tokyo due to the radiation caused by leaks in the country nuclear power plants. Such a fear of radiation that would spread into the ocean, plants, humans and animals which caused a great economic loss. The fear of radiation exposure

continues to haunt the people of Japan and around the world. In addition there was no protection for the workers in the plant to stop the radiation leaks in time to minimize the damage and economic loss. With this invention the workers now can be protected and can do their job under hardiest conditions as they will not develop any type of cancer.

In addition with this invention it is possible that the nuclear power industry with hundreds of billions at stake could be saved if the workers are protected then can operate the power plant. Not only the human beings can be protected from the radiation exposure, but also food and animals can be protected as well. (Under another patent application, internal number RAA025)

In vitro Studies have been performed for: Plasma Products

Animal derived products Recombinant Products Monoclonal Products Cell Expression products PLASMA PRODUCTS

IN VITRO STUDIES FOR HIV VIRUS 1 & 2

HIV Study Report

PROJECT ID: RAAS<201110178

STUDY TITLE: In vitro Anti HIV Activity of Human Plasma Derived Proteins on HIV RT Enzyme

STUDY PERIOD: Nov. 16-Nov. 21, 2011

REPORTING DATE: Nov. 24, 2011

The research service was conducted in accordance with sound scientific principles. This report accurately reflects the raw data from the assay.

I. Study Objective:

To analyze human plasma derived proteins for anti HIV activity on HIV RT enzyme

II. Study Protocols:

1. Materials:

1.1 Samples information: RMS provided the test articles in the form of dry powder or liquid (Table

1). Wuxi provided reference compound in Drv1SO solution.

TABLE 1
Sample information
AFCC RONA 0.00001% Lyophilized AFOD KH 10 ml

| Name | Protein conc. | Formulation | Diluents |
|---|---|---|---|
| AFOD KH | 10% | Liquid | |
| AFCC KH | 3.50% | Liquid | |
| AFCC RASS1 | 4% | Lyophilized | AFOD KH 10 mL |
| AFCC RASS4 | 0.0020% | Lyophilized | AFOD KH 10 mL |
| AFCC RONA | 0.00001% | AFOD KH 10 ml | AFCC RONA |
| | Lyophilized | | |

1.2 Reagents:

TABLE 2
List of reagents

| Reagents/Plates | Vendor | Cat. # |
|---|---|---|
| HIV-1 Reverse Transciptase | Merck | 38129-SOOU |
| Wild type enzyme | | |
| Avidin standard plates | | MSD-L15AA-6 |
| RNA template t500 syntheic | IB/GMBH | Cat. #89142N/S |
| piece of RNB | | |
| CHAPS | Pierce | Pirece-28300 |
| EGTA | Sigma | Sigma-E3889-10G |
| DTT | Sigma | Sigma-D43815-SG |
| d-ATP | Sigma | Sigma-D6500-10MG |
| d-GTP | Sigma | D4010-10MG |
| d-CTP-Na2 | Sigma | D4635-10MG |
| Water (DEPC treated) | Invitrogen | Invitrogen-750023 |
| dry bipD500 primer | Shanghai | |
| | Shenggong | |
| BSA | Sigma | Sigma-A3294 |
| 4-Read buffer T | MSD | MSD-R92TD-1 |
| Ru - d- UTP | MSD | Lot: DG2005245071 |
| 96-well round bottom | Costar | Costar-3365 |
| polypropylene plates | | |
| PCR tubes | AXYGEN | AXYGEN-PCR-0208-C |
| PCR tube covers | AXYGEN | AXYGEN-PCR-2CP-RT-C |

1.3 Instrument

Sector Imager 56000 (MesoScale Discovery MSD) Eprnotoin (Eppendorf)

Janus (perkinelrner)

Orbital shaker

2. Methods

2.1 !C50 measurement

2.2.1 Drug treatment: Human plasma derived protein dilutions are made by using EpMotion with 2-fold serial dilutions for 10 concentrations, each in duplicate.

a) Add 30 !JL of enzyme solution per well of the Costar 96 well plates. b) Add 5 !JL of test article or PBS or DMSO.

c) Seal plate and shake for 2 minutes on an orbital shaker

d) Incubate for 30 minutes on an orbital shaker at room temperature. e) Add •15 !JL of the Master Mix to initiate the reaction.

f) Seal plate and shake for 5-10 minutes.

g) Incubate at 37 degree for 90 minutes.

h) While this is incubating, add 100 iJL of 5% BSA in PBS to the wells of the avidin plates.

i) Seal the avidin plates and incubate for 1 hour at room temperature.

j) After the 90 minute incubation, add 60 pl of quenching buffer to the reaction wells. k)

Seal the plates and incubate for 5 minutes on the plate shaker.

I) Transfer 50 iJL of the well contents to MSD blocked plates (the blocking buffer is simply dumped off. No wash is needed).

m) Incubate MSD plates at RT for 60 minutes.

n) Freshly dilute the 4× read buffer T to 1× using distilled water (not DEPC-treated)

o) Wash rv1SD plates 3 times with 150 pl of PBS per well per wash. p) Add 150 iJL of 1× read buffer T to tile wells.

q) Read on the Sector Imager Instrument.

2.2.2 Sample or Compound addition

Test samples were diluted in PBS as 3.5×104 pg/ml stocks. Sample dilutions are made by using Epmotion with 2-fold serial dilutions for 10 concentrations plus PBS (see below for final compound concentrations in the HIV-RT enzyme assay). Reference compound were dissolved in DMSO as “iO mM stocks and dilutions are made by using Epmotion with 3-fold serial dilutions for 10 concentrations plus Drv1SO (see below for final compound concentrations).

TABLE 3
Sample or compound concentrations for !C50 measurement

| Name | | | | | | | | | | |
|---|---|---|---|---|---|---|---|---|---|---|
| | Concentration (ug/ml) | | | | | | | | | |
| AFOD KH | 400 | 2.00 | 100 | 50 | 2.5 | 12.5 | 6.25 | 3.1 | 1.6 | 0.8 |
| AFCC KH | 400 | 2.00 | 100 | 50 | 2.5 | 12.5 | 6.25 | 3.1 | 1.6 | 0.8 |
| AFCC RAASl | 400 | 2.00 | 1.00 | 50 | 2.5 | 12.5 | 6.25 | 3.1 | 1,6 | 0.8 |
| AFCC RAAS 4 | 400 | 2.00 | 1.00 | 50 | 2.5 | 12.5 | 6.25 | 3.1 | 1,6 | 0.8 |
| AFCC RDNA | 400 | 2.00 | 1.00 | 50 | 2.5 | 12.5 | 6.25 | 3.1 | 1,6 | 0.8 |
| | Concentration (nM) | | | | | | | | | |
| Reference | 100 | 33.3 | 11.1 | 3.7 | 1.2 | 0.4 | 0.1 | 0.05 | 0.02 | 0.01 |
| Compound | | | | | | | | | | |

2.2.3 Data analysis:

Percent of HIV-RT inhibition by protein or compound is calculated using the following equation:

% lnh.=[1−(Signal of sample−Signal of control)/(Signal of DMSO or PBS control−Signal of control)1*100.

Dose-response curves are plotted using Prism

III. Assay results:

3.1 Raw data from the HIV-RT enzyme assay.

3.1.1 HIV-RT enzyme assay Plate Map*:

| column | column | column | colurnn | column | colurnn | coiurnn | column | column | colurnn | column | colurnn |
|---|---|---|---|---|---|---|---|---|---|---|---|
| 1 | 2 | 3 | 4 | s | 6 | 7 | 8 | 9 | 10 | 11 | 12 |

Plate 1

| * | raw A | p | AFOD KH | B |
|---|---|---|---|---|
| | raw B | B | | G |
| | raw C | S | AFCC KH | |
| | raw D | | | |
| | raw E | p | AFCC RAAS 1 | p |
| | raw F | B | | B |
| | raw G | S | Reference Compound | S |
| | raw H | | | |

Plate 2

| raw A | p | AFCC RAAS 4 | B |
|---|---|---|---|
| raw B | B | | G |
| raw C | S | AFCC BONA | |
| raw D | | | |
| raw E | p | Reference Compound | p |
| raw F | B | | B |
| raw G | S | Dtv1SO | s |
| raw H | | | |

BG: background

* BG: background

3.1.2 Raw data

| column | coiumn | coiumn | column | column | column | column | column | column | column | column | coiumn |
|---|---|---|---|---|---|---|---|---|---|---|---|
| 1 | 2 | 3 | 4 | 5 | 6 | 7 | 8 | 9 | 10 | 11 | 12 |

Plate 1:

| | | | | | | | | | | | |
|---|---|---|---|---|---|---|---|---|---|---|---|
| 2439 | 1596 | 2113 | 2160 | 2304 | 2448 | 2214 | 2152 | 2307 | 2360 | 2357 | 60 |
| 2569 | 1866 | 2154 | 2343 | 2351 | 2371 | 2397 | 2317 | 2310 | 2454 | 2245 | 64 |
| 2571 | 281 | 329 | 393 | 563 | 805 | 1157 | 1683 | 2011 | 2304 | 2384 | 60 |
| 2361 | 267 | 306 | 376 | 518 | 762 | 1156 | 1600 | 1912 | 2158 | 2185 | 58 |
| 59 | 1238 | 1782 | 2097 | 2230 | 2299 | 2326 | 2374 | 2368 | 2329 | 2449 | 2267 |
| 52 | 1248 | 1812 | 2166 | 2300 | 2406 | 2462 | 2398 | 2369 | 2346 | 2353 | 2366 |
| 54 | 87 | 142 | 246 | 469 | 850 | 1241 | 1629 | 1791 | 1873 | 1851 | 2263 |
| 53 | 85 | 132 | 241 | 474 | 833 | 1349 | 1651 | 18B | 1924 | 1907 | 2438 |

Plate 2:

| | | | | | | | | | | | |
|---|---|---|---|---|---|---|---|---|---|---|---|
| 2491 | 1713 | 1940 | 2168 | 2411 | 2358 | 2378 | 2459 | 2289 | 2262 | 2038 | 43 |
| 2596 | 1674 | 2220 | 2344 | 2547 | 2491 | 2418 | 2541 | 2443 | 2476 | 2104 | 45 |
| 2539 | 1147 | 2176 | 2381 | 2522 | 2388 | 2433 | 2314 | 2459 | 2358 | 2369 | 44 |
| 2544 | 1689 | 2123 | 2305 | 2453 | 2385 | 2400 | 2426 | 2204 | 2049 | 2168 | 39 |
| 44 | 91 | 146 | 270 | 514 | 957 | 1429 | 1801 | 1807 | 1895 | 1880 | 2142 |
| 38 | 85 | 139 | 263 | 472 | 946 | 1377 | 1614 | 1708 | 1850 | 1853 | 2292 |
| 45 | 2119 | 2160 | 2084 | 2046 | 2069 | 1963 | 1975 | 2002 | 1961 | 1912 | 2343 |
| 43 | 2052 | 2038 | 2039 | 1975 | 1954 | 1860 | 1968 | 1972 | 1875 | 2042 | 2405 |

3.2 Activity of the Samples or compounds. IC50 values are summarized in Table 4.

GraphPad

Prism files containing dose-dependent curves are presented in this report, as shown in FIG. 1.

TABLE 4
!C50 Summary of the the human plasma derived
proteins and the reference compounds.

| Name | IC50 (ug/ml) |
|---|---|
| AFOD KH | >400 |
| AFCC KH | 9.89 |
| AFC RASS1 | 49% inhibition at 400 ug/ml |
| AFCC RASS4 | >400 |
| AFCC RDNA | >400 |
| | IC50 (nM) |

| | | |
|---|---|---|
| Reference | 0.9 | 1.2 |

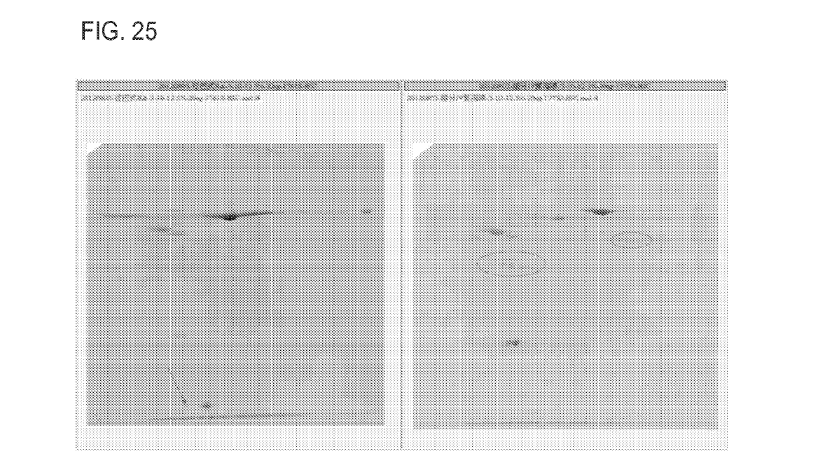

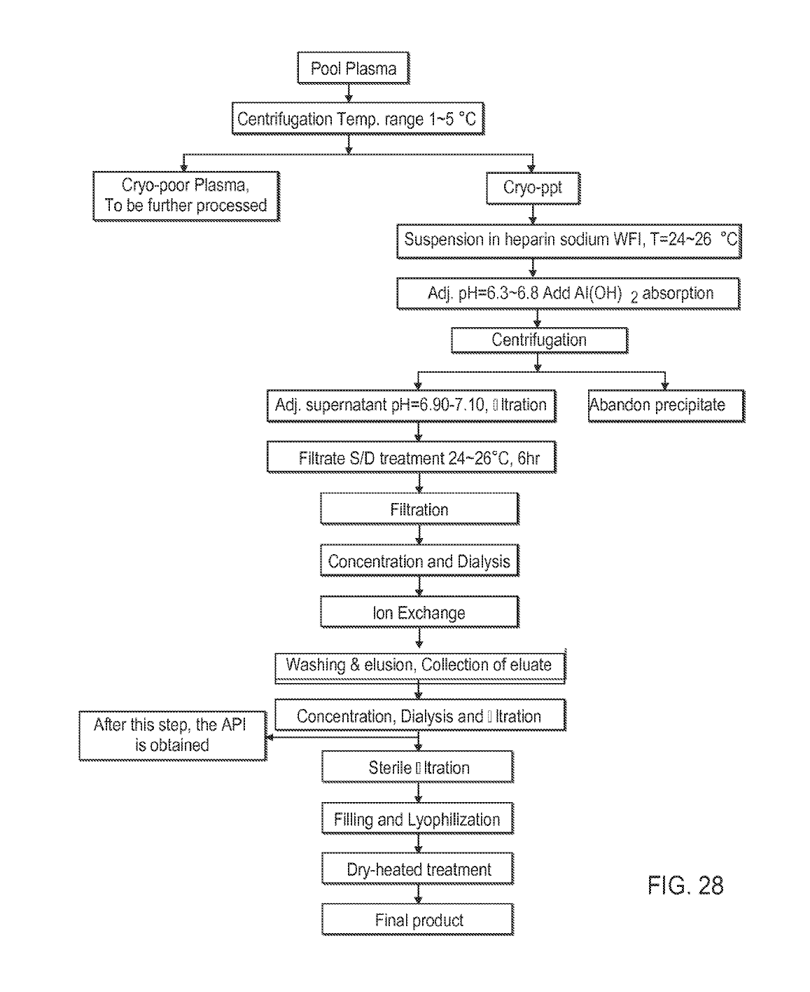

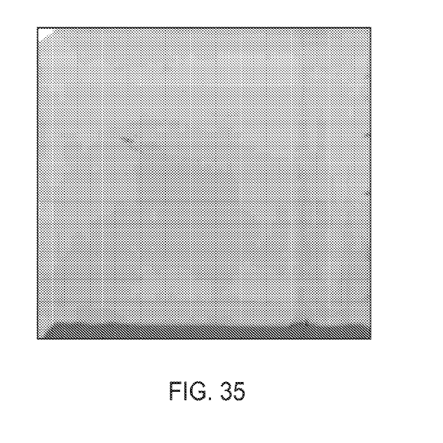

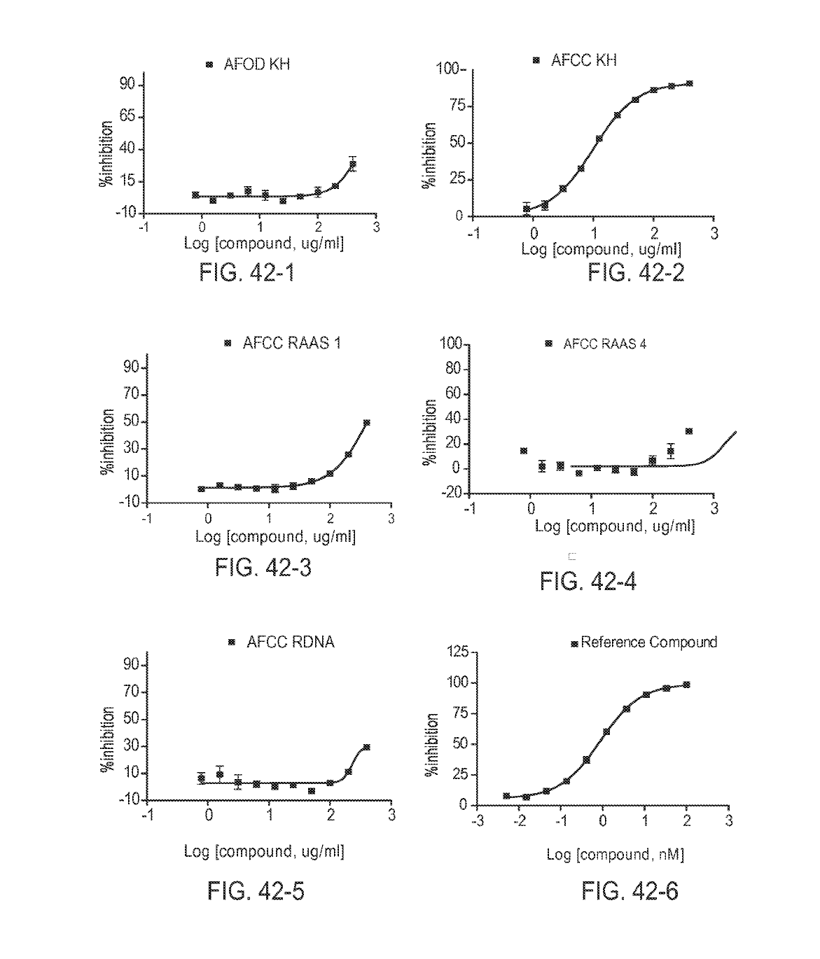

FIG. 42-1 through 42-6. Dose-dependent curves (by GraphPad Prism}

4. Conclusions

The Z factors of the two plate were 0.84 (plate 1), 0.80 (plate 2), which were much better than QC standard of OS Therefore, the assay data met our QC qualification.
The IC50 s of positive control in this study were 0.9 nM (plate 1), 1.2 n1\ !1 (plate 2) and these results are consistent with our previous data.

IN VITRO STUDIES OF HEPATITIS B VIRUS HBV Study Report

PROJECT CODE: RAAS 20110815C

STUDY TITLE: To analyze human plasma derived proteins for anti HBV activity in HepG2.2.15 cells

STUDY PERIOD: Nov. 24-Dec. 6, 2011

REPORTING DATE: Dec. 23, 2011

L Study Objective: To test human plasma derived proteins for anti-HBV potency and cytotoxicity in a stable HBV cell line

II. Study Protocols:

1. Materials:

Cell line: HepG2.2.15

1.2 Samples:

RAAS provided the test articles in the form of dry powder or liquid {Table 1pi::st samples were diluted in PBS as 3.5×1041Jg1 ml stocks. Sample dilutions are made by Janus with 2-fold serial dilutions for 8 concentrations plus PBS. Lamivudine is diluted with 3-fold for 9 concentrations.

TABLE 1
Sample information

| | Protein | | |
|---|---|---|---|
| Name | cone. | Formulation | Diluents |
| AFOD KH | 10% | 9 | |
| AFCC KH | −5.50% | Liquid | |
| AFCC RAAS 1 | 4% | Lyophilized | AFOD KH 10 mL |
| AFCC RAAS 4 | 0.0020% | Lyophilized | AFOD KH 10 mL |
| AFCC RDNA | 0.00001% | Lyophilized | AFOD KH 10 mL |

1.3 ECso and CCso measurement Test human plasma derived proteins in the stable HBV cell line

HepG2.2.15 for anti-HBV potency.

i) Cell culture medium: RPM 1640-4% FBS-1% PeniStrep-1% Glutamine

ii) HepG2.2.15 cell culture: Grow the cells in T75 flask. Incubated at 3TC, 950 ft, humidity, 5% C02. Perform 1:3 split every 2-3 days. iii) EC5o measurement:

1) Drug treatment

a) Human plasma derived protein dilutions are made by using Janus with 2-fold serial dilutions for 9 concentrations, each in duplicate.

b) Check cells under microscope.

c) Prepare cell suspension and count cell number. d) Seed the HepG2.2.15 cells into 96-well plates.

e) Treat the cells with cell culture medium containing individual human plasma derived protein 24 hours after cell seeding, the final concentrations of the samples are shown in Table 2.

Name

| | Concentration (ug/ml) | | | | | | | | | |
|---|---|---|---|---|---|---|---|---|---|---|
| AFOD KH | 400 | 2.00 | 100 | 50 | 25 | 12.5 | 6.25 | 3.1 | 1.6 | 0.8 |
| AFCC KH | 400 | 2.00 | 100 | 50 | 25 | 12.5 | 6.25 | 3.1 | 1.6 | 0.8 |
| AFCC RAAS 1 | 400 | 2.00 | 100 | 50 | 25 | 12.5 | 6.25 | 3.1 | 1.6 | 0.8 |
| AFCC RAAS 4 | 400 | 2.00 | 100 | 50 | 25 | 12.5 | 6.25 | 3.1 | 1.6 | 0.8 |
| !\FCC RDNA | 400 | 200 | 100 | 50 | 25 | 12.5 | 6.25 | 3.1 | 1.6 | 0.8 |
| | Concentration (uM) | | | | | | | | | |
| Lamivudine | 2 | o.6667 1 | o.nn 1 | o.o741 | 0.0247 | o.oo82 1 | oooon | o.ooog 1 | o.oo03 1 | o.oom i |

f) Refresh protein-containing medium on day 3 of drug treatment g) Collect culture media from the HepG2.2.15 plates on day 6 followed by HBV DNA extraction using QIAamp 96 DNA Blood Kit (QIAGEN #51161).

2) Real time PCR for HBV DNA quantification. a) Dilute HBV plasmid standard by 10-fold from 0.1 ng/ul to 0.000001 ng/ul. b) Prepare realtime PCR mix as shown blow.

| | | | Volume for 100 | |
|---|---|---|---|---|
| PCR reagents | Volume | | Reactions | |
| DEPC Water | 1.i | ul | •11O | ul |
| Taqman Universal Master | 12.5 | ul | 1250 | ul |
| Mix(2X) | | | | |
| HBV Primer ForNard(50 uM) | 0.2 | ul | 20 | ul |
| HBV Primer Reverse(50 uM) | 0.2 | ul | 20 | ul |
| HBV Probe(5 uM) | 1 | ul | “IOO | ul |
| Total | 15 | ul | i50 | ul |

c) Add 15 ul/well PCR mix to 96-well optical reaction plates.

d) Add 10 ul of the diluted plasmid standard to C12-H12. The amount of HBV DNA in each standard well is: ing, 0.1 ng, 0.01 ng, 0.001 ng, 0.0001 ng, and 0.00001 ng, respectively.

e) Transfer 10 ul of the extracted DNA to the other wells (from Row A-H to the corresponding wells in the optical reaction plates). f) Seal the plates with optical adhesive film. g) Mix and centrifuge. h) Place the plates into realtime PCR system and set up the program according to the

table blow.

| | | |
|---|---|---|
| 50′C. | 2 rnin | 1 cycle |
| 95′C. | 10 min | 1 cycle |
| | 15 s | 40 cycle |
| 60′C. | 60 s | |

3) Data analysis: A standard curve is generated by plotting Ct value vs. the amount of the HBV plasmid standard, and the quantity of each sample is estimated based on the Ct value projection on the standard curve; percent of HBV inhibition by protein or compound is calculated using the following equation: % lnh.=[1−(HBV quantity of sample−HBV quantity of HepG2 control)/(HBV quantity of 0% Inhibition control−HBV quantity of HepG2 control)]*100.

Test human plasma derived proteins in the stable HBV cell line HepG2.2.15 for cytotoxicity i)
Cell culture medium: RPM 1640-4% FBS-1% Pen/Strep-1% Glutamine

ii) HepG2.2.15 cell culture: Grow the cells in T75 flask. Incubated at 3TC, 95% humidity, 5% C02. Perform 1:3 split every 2-3 days. iii) CC5o measurement

a) Human plasma derived protein dilutions are made by using Janus with 2-fold serial dilutions for 9 concentrations, each in duplicate. b) Check cells under microscope.

c) Prepare cell suspension and count cell number. d) Seed the HepG2.2.15 cells into 96-well plates.

a) Treat the cells with cell culture medium containing individual human plasma derived protein 24 hours after cell seeding, the final concentrations of the samples are shown in Table 2.

e)

f) Refresh protein-containing medium on day 3 of drug treatment.

g) Test cell cytotoxicity on day 6 using CellTiter-Blue Cell Viability Assay KIT.

iii. Assay results:

TABLE 3
ECso raw data (Plate 1, DNA quantity, ng)

| | Sample final dose (ug/ml) | | | | | | | | | |
|---|---|---|---|---|---|---|---|---|---|---|
| | 400 | 200 | 100 | 50 | 255 | 12.5 | 6.25 | 3.13 | 1.56 | 0% |
| AFOD | 0.GOG | 0.005 | 0.005 | 0.006 | 0.007 | 0.006 | 0.006 | 0.007 | 0.007 | 0.007 |
| KH | | | | | | | | | | |
| AFCC KH | 0.006 | 0.008 | 0.007 | 0.007 | 0.007 | 0.OOG | 0.OOG | 0.002 | 0.007 | 0.002 |
| AFCC 1H | 0.009 | 0.009 | n.OO′i | n.OO′i | 0.006 | 0.006 | 0.006 | o.out. | (IUOi) | o.out. |
| AFCC | U.006 | 0.OOb | 0.OOb | 0.OOb | 0.OOti | 0.(ll)i> | 000;′ | (IUOG | 0.007 | (IUOG |
| RAAS 1 | | | | | | | | | | |
| AFCC | 0.00′3 | 0.OOG | 0.oo:: | 0.OOG | 0.(11)9 | 0.(Ilk | 0(lQ(i | (Iuo:; | 0.008 | (Iuo:; |
| RAAS 1 | | | | | | | | | | |

TABLE 4
EC5o raw data (Plate 2, DNA quantity, ng)

| | Sample final dose (ug/ml) | | | | | | | | | |
|---|---|---|---|---|---|---|---|---|---|---|
| | 400 | 200 | 100 | 50 | 255 | 12.5 | 6.25 | 3.13 | 1.56 | 0% |
| AFOD KH | 0.00!3 | 0.G08 | 0.G07 | 0.G07 | 0.G09 | 0.G09 | 0.G09 | 0.012 | 0.00!3 | 0.G08 |
| AFCC KH | 0.007 | 0.00′1 | 0.00′1 | 0.00′1 | 0.00′1 | 0.008 | 0.00′1 | 0.008 | 0 | 0.007! |
| | | | | | | | | | | 0.006 i |
| | | | | | | | | | | 0.006 |
| AFCC 1H | .007 | 0.007 | 0.GOG | 0.G07 | 0.G07 | 0.GOG | 0.G07 | 0.GOG | 0.G07 | .007 |
| AFCC | 0.001 | 0.0( )1 | 0.G01 | 0.G02 | 0.G03 | 0.G05 | 0.G07 | 0.G11 | 0.G10 | 0.001 |
| RAAS 1 | | | | | | | | | | |
| AFCC | 0.001 | 0,001_{—} | 0.00\ | 0.002 | U,004 | U,OO′l | U,010 | 0.012 | U,014 | 0.001 |
| RAAS 1 | | | | | | | | | | |

TABLE 5
CCso raw data (Plate 1)

| | | | | | | | | | | | | |
|---|---|---|---|---|---|---|---|---|---|---|---|---|
| 5,a:;;-lp le 1- | !\ | 400 | 200 | JOO | fiO | 2S | L2.50 | 6.25 | 3.t,) | 1.SG | | DMEM |
| LnoLclose· | | | | | | | | | | | | |
| (ug/ml) | | | | | | | | | | | | |
| .“>FOD KH | B | 5580:\ | 64r{9′l | ? l230 | rf2l, 9 | rnl39 | “/8!39 | ?Wt0 | ?9l6l | “!9i!·12 | 8l56l | llil8 |
| AFDD 1\H | \, | 56.S2:\ | 6(;:33 | ?063l | nl31 | r{f,( )(;8 | “/30 ll | Tf9!J6 | ?i!·120 | ?!J1Eo2 | XX8l68“1 | llil3 |
| ,L>,FCC KH | | 82ns | EA496 | g::S96 | 8m:n | 193.:J4 | ,s1008 | 809·? | E\089Eo | Tf356 | ?90:34 | ll 93 |
| AFCC KH | E | 815013 | 1′7561 | “{t1728 | 30·1OJ | 73910 | 82101 | 8:35fl7 | 1′601′7 | “lr!99l | 32662 | 1168 |
| AFCC RAr′\S1 | F | 66408 | 74141 | 78364 | 78223 | 76486 | 77972 | 75031 | 78457 | 66609 | 70886 | llGl |
| AFCC Rl\AS·j | 6T?46 | 17(!)\) | ?4032 | rfSl93 | “(8[”!9 | “/66′1 | 803130 | 19r{!)′l | 694?:3 | TI56.:J | ll“/(l | AFCCRl\AS·j |
| H | | | | | | | | | | | | |

Note:
DrvlEI\!1-′100·;; inhibition control

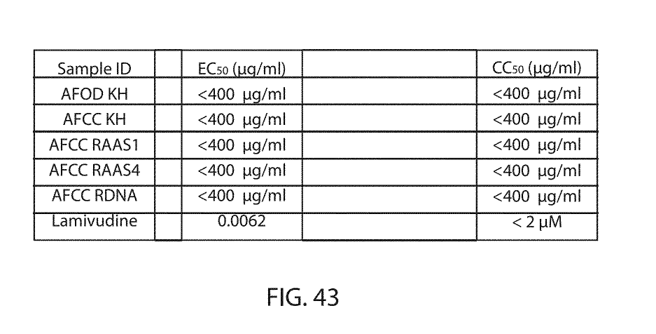

FIG. 43: Table 7. EC5o and CC5o summary

IV. Conclusions

The EC5D of the positive control lamivudine in this study is 0.0062 ul\! 1, which is consistent with our previous data.

IN VITRO STUDIES OF HEPATITIS C VIRUS

HCV Study Report

PROJECT CODE: RASSD20111017A

STUDY TITLE: Test human plasma derived proteins against HCV genotype 1a, 1b and 2a replicons for antiviral activity (EC50)

STUDY PERIOD: Nov. 16-Nov. 21, 2011

REPORTING DATE: Nov. 24, 2011

The research service was conducted in accordance with sound scientific principles. This report accurately reflects the raw data from the assay.

I. Study Objective:

To analyze human plasma derived proteins for anti HCV activity (EC50) and cytotoxicity (CC50) using HCV 1a, 1b and 2a replicon culture systems

II. Study Protocols:

3. Materials:

1.1 Cell Une:

Replicon cell lines 1a and 2a were established following published methods (1,2) using Huh? by G4″18 selection. The replicons were assembled using synthetic gene fragments. The GT 1a line is derived from H77 and contains PVIRES-Luciferase-Ubi-Neo, and two adaptive mutations: P1496L, 822041. The 2a line contains no adaptive mutations and encodes a Luciferase reporter. The 1b replicon plasmid is also assembled using synthetic gene fragments. The replicon genome contains PVIRE8-Luciferase Ubi-Neo gene segments and harbors 1 adaptive mutation (822041), and the backbone is Con1.

1.2 Compounds:

The test articles are supplied in the form of dry powder or 10 mM solution, and Ribavirin as control, in duplicate.

1.3 Reagents:

TABLE 1
List of reagents

| | | REAGENT |
|---|---|---|
| REAGENT | VENDOR | Catalog Number |
| ! Dimethyl sulfoxide (mv1SO) | Sigma | Cat#34869 |
| 1---o fEM---------------------------------------- | - --- | ---cai#T1-96o o-ii r- |
| ------------------------------------------------- | fr1v_i_tro | ----------- |
| : | _9_e_n | |
| ! Fetal Bovine Serum (FBS) | Gibco | Cat#16140 |
| ---------------------------------------------------- | ------------------------ | ------------------------- |
| ---------------------------------------------------- | -------- | ------------------------- |
| --- | Invitrogen | - |
| 1 Penicillin-Streptomycin | | Cat#15070063 |
| ! MEM non-essential amino acids | Invitrogen | cat#11140-050 |
| [---c=8iLiTa_m_iile---------------------------- | --TilvTtro_9_e_n | ---caw25o3o o-sT |
| -------------------------------------------------- | | - |
| i | | |
| ! Trypsin/EDTA | Invitrogen | Cat#25200-072 |
| ---------------------------------------------------- | ------------------------ | ------------------------- |
| ---------------------------------------------------- | -------- | ------------------------- |
| --- | Hyclone | - |
| 1 DPBS/Modified | | SH30028.01B |
| ! 96 well cell plate | Greiner | Cat#655090 |
| :--- | ---caw-i3-6os T------ | |
| | rro_m_e_{—} | |
| | 9_a | |
| ! Bright-Gio Promega | Cat#E2650 | |

1.4 Instrument

to Envision(Perkinelmer)

to Multidrop(Thermo)

to Janus (Perkinelmer)

4. Methods

2.1 Cell Addition

T150 flask containing 1a, 1b and 2a replicons cell monolayer is rinsed with 10 ml pre-warmed PBS. Add 3 ml of pre-warmed Trypsin 0.25% and incubate at 5% C02, 37 cC for 3 minutes.

Nine milliliters of DMEM complete media are added, and the cells are blown for 30 s by pipetting. The cells are counted using hemocytometer.

1a, 1b and 2a replicons cells are resuspended in medium containing 10% FBS to reach a cell density of 64,000 cells/ml (to obtain a final cell plating density of 8000 cells/125 ul/well). Plate cells in Greiner 96 black plate using Multidrop. Incubate plate at 5% C02, 37 t for 4 hours.

2.2 Compound addition

RAAS provided the test articles in the form of dry powder or liquid (Table 2). Test samples were diluted in PBS as 3.5×10\Jg/rnl stocks. Sample dilutions are made by Janus with 2-fold serial

dilutions for 10 concentrations plus PBS. Ribavirin is also diluted by Janus with 2-fold for 10 concentrations. The final sample concentrations of tile HCV replicon assay are described in Table 3.

TABLE 2
Sample information

| Name | Protein cone. | Formulation | Diluents |
|---|---|---|---|
| AFOD KH | 10% | Liquid | |
| AFCC KH | 3.50% | Liquid | |
| AFCC RAAS 1 | 4% | Lyophilized | AFOD KH 10 ml |
| AFCC RAAS 4 | 0.0020% | Lyophilized | AFOD KH 10 ml |
| AFCC RONA | 0.00001% | Lyophilized | AFOD KH 10 ml |

TABLE 3
Sample or compound concentrations for EC50 and CC50 measurement

| | HCV | | | | | | | | | | |
|---|---|---|---|---|---|---|---|---|---|---|---|
| Name | Genotype | | | | | | | | | | |
| | | Concentration (pg/ml) | | | | | | | | | |
| AFOD KH | 1a/1b/2a | 400 | 200 | 100 | 50 | 25 | 12.5 | 6.3 | 3.1 | 1.6 | 0.8 |
| AFCC KH | | 400 | 200 | 100 | 50 | 25 | 12.5 | 6.3 | 3.1 | 1.6 | 0.8 |
| AFCC RAAS 1 | | 400 | 200 | 100 | 50 | 25 | 12.5 | 6.3 | 3.1 | 1.6 | 0.8 |
| AFCC RAAS 4 | | 400 | 200 | 100 | 50 | 25 | 12.5 | 6.3 | 3.1 | 1.6 | 0.8 |
| AFCC RONA | | 400 | 200 | 100 | 50 | 25 | 12.5 | 6.3 | 3.1 | 1.6 | 0.8 |
| | | Concentration (IJM) | | | | | | | | | |
| Ribavirin | | 320 | 160 | 80 | 40 | 20 | 10 | 5 | 2.5 | 1.3 | 0.6 |

2.3 Detection (after 72 hours of incubation)

Bright-Gio Luiferase and CellTiter-Fluor′M are prepared and stored in dark while allowing to equilibrate to room temperature. Plates are removed from incubator to allow equilibration to room temperature. Multidrop is used to add 40 ul CellTiter-Fluor′″ to each well of compound-treated cells.

The plates are incubated for 0.5 hour, and then read on an Envision reader for cytotoxicity

calculation. The cytotoxicity is calculates using the equation below.

-

;O y O.OX1C1/

Cmpd—Background

D1\fS0—Background

xlOO

100 ul of Bright-Gio are added to each well, incubated for 2 minutes at room temperature, and chemi-luminescence (an indicator of HCV replication) is measured for EC50 calculation.

The anti-replicon activity (% inhibition) is calculated using the equation below ( )/( )Jnhibition===1 --- --- !!2 ::; . . . : - - !I_?

--- 100

D}vfS′O—back,ground

Dose-response curves are plotted using Prism.

III. Assay: results:

1 Assay Plate Map

plate •1

| | | |
|---|---|---|
| C | AFOD KH | P |
| T | AFCC KH | B |
| L | AFCC RAAS 1 | S |

plate 2 columncolumncoiumncolumncolumncolumnwlumn
coiumncolumncolumncolumn column

| | | |
|---|---|---|
| C | AFCC RAAS 4 | P |
| T | AFCC RONA | B |
| L | Ribavirin | S |

2 Raw data

2.1 Raw data of cytotoxicity assay

| | | | | | | | | | | | |
|---|---|---|---|---|---|---|---|---|---|---|---|
| 11788 | 3?82D | 7G241 | ?9783 | 8l′l094 | 89352 | 8G4?5 | 84132 | 79122 | 8231? | 78529 | 84888 |
| 10513 | 38733 | 73718 | 79841 | 90368 | 82949 | 84058 | 85256 | 86834 | 85378 | 81751 | 78143 |
| 11907 | 71545 | 83521 | 89′104 | 9183′1 | 87528 | 88304 | 89908 | 89782 | 81452 | 87404 | 80906 |
| 10873 | 82130 | 82349 | 86032 | 91782 | 13224 | 90052 | 88416 | 8502P | 87835 | 82113 | 80·121 |
| 1201; | G1801 | 825?4 | 7i31G | 91001 | i01iD | 94232 | 932D3 | i04W | 91lG4 | 85286 | 7′i43i |
| 10586 | 51803 | 75949 | 84140 | 89954 | 84298 | 85969 | 87016 | 87714 | 84577 | 81008 | 81025 |
| 12214 | 59805 | 68928 | 67259 | 68991 | 70963 | 70986 | 72721 | 80578 | 72648 | 86545 | 75138 |
| 10586 | 55271 | 62901 | 59758 | 63586 | 63753 | 510′14 | 64486 | 70755 | 74224 | 8488′1 | 74471 |
| 121f37 | 75390 | 86019 | 93902 | 94512 | 84075 | 78058 | 81G19 | 7841P | 813′11 | 8′1G04 | 83′171 |
| 10838 | 79348 | 85248 | 88417 | 90128 | i098l | 81205 | 87054 | 8037P | 82′154 | ?9328 | 84·191 |
| 1200G | 42127 | ′i6fr16 | 5S340 | 70tFG | ′133-m | 84894 | 85941 | 8?58′1 | 9W10 | 91748 | 7D542 |
| 10398 | 52814 | 54925 | 59760 | 72108 | 85112 | 88.015 | 84100 | 88429 | 87978 | 88712 | 79154 |

| | | | | | | | | | | | | |
|---|---|---|---|---|---|---|---|---|---|---|---|---|
| 11859 | 51104 | 57291 | 50533 | 71572 | 7·1590 | 7·.1590 | 72696 | 63905 | 67′104 | 54951 | 63293 | 68405 |
| 10705 | 46415 | 52869 | 63478 | 66044 | 76232 | 76232 | 75102 | 64′:101 | 70704 | f34733 | 73663 | 65861 |
| 11915 | 48782 | 62222 | 70988 | 7006·! | 72337 | 72337 | 70822 | 62570 | 61489 | f3′:1424f38 | 67863 | 62024 |
| 10fj98 | 54?87 | f$′7780 | 7′4332 | ′77817 | ?fj2( )f$ | ?fj2( )f$ | 71439 | 69920 | tm2oD | ′i73 | l10′i5 | 7l)183 |
| 11617 | 56776 | 72151 | 78099 | 73707 | 80133 | 80133 | 77881 | 71345 | 74569 | 75191 | 72729 | 67333 |
| ′10389 | 55289 | 73692 | 79149 | 720fJ8 | 79174 | 79174 | 80854 | 75314 | 7fJ363 | 74574 | 59452 | 70933 |
| 1F81 | 46220 | 70386 | 71631 | 740381OOP! | 70501 | 65402 | 59277 | 577′14 | 59415 | 60015 | 55776 | |
| | | | | | | | f349f38 | | | | | |
| | | | | | | | f3294B | | | | | |
| 10f359 | 50913 | 63077 | 71054 | ?0043fj5994 | 6627? | | 63481 | 68110 | 7H346 | 58898 | 58925 | |
| 11Sl0 | | 37580 | 66840 | 4859, | 1)6523 | | 62875 | | 67B81 | 1)9418 | | |
| 10463 | 59788 | 35505 | 38330 | 43076 | 75550 | G02f33 | 65543 | 64S91 | 64326 | 61607 | | |
| 112.15 | 31282 | 70386 | ,m247 | 740381OOP·? | 59252 | 68223 | 59277 | 63360 | 6681, | 58225 | 64260 | |
| | | | | | | | f349f38 | | | | | |
| | | | | | | | f3294B | | | | | |
| 10340 | 34855 | 63077 | 71631f33452 | ?0043fj5994 | 5620f1 | 61155 | 63481 | 64284 | 66557 | 56655 | 60285 | |
| 11260 | 62423 | 63994 | 60008 | 66320 | 63246 | 63076 | 62824 | 5422fj | 5422fj | 52388 | 56f;80 | 52388 |
| 10127 | 54433 | 51255 | 51,m7 | 55262 | 59280 | 558fJ0 | 50222 | 55138 | 55138 | 55625 | 575,2 | 56526 |
| 11453 | 52361 | 58693 | f32869 | 69429 | 56045 | 58716 | 5B284 | f30293 | f30293 | 637?8 | 5811? | 63778 |
| | | | | | 1′l34S1 | | | | | | | |
| 10728 | )f$90B | f3SS47 | fY7010 | 64930 | 60082 | G4533 | f33630 | | | 64781 | 1)4208 | G47B1 |
| | | | | | 1B244 | | | | | | | |
| 11424 | 50095 | 64112 | 61153 | 63665 | 63246 | 61140 | 62072 | | | 68446 | 61890 | 58446 |
| 10165 | 52406 | 60200 | 68101 | 64203 | 59280 | 61168 | 64479 | 66478 | 66478 | 64375 | 6130fj | 64375 |
| 12001 | 668fJ8 | 51275 | 50,53 | 63884 | 6·1264 | 60534 | 50138 | 50138 | 5546fl | 62475 | 68167 | 66469 |
| 10936 | 66043 | f30181 | 55?62 | 59218 | 56456 | 64f353 | 56607 | f31353 | 60143 | 60143 | 56251 | 61353 |
| | | | | | 1)2106 | | | | | | 1)0706 | |
| | | | | | 1)9f348 | | | | | | 1)69?5 | |
| 117S1 | G0500 | St1343 | Gf3462 | 644?0 | 6017f; | G33f34 | St1872 | 65B81 | 62280 | 62280 | 70185 | G58B1 |
| | G4051) | GH127 | | 60913 | 59597 | Gl701 | | 65950 | 64i31 | 64i31 | 5945fl | |
| 10f313 | 37011 | 43034 | 47350 | 54734 | 56456 | 68095 | f3?3S9 | 68319 | 70444 | 70444 | 56251 | |
| | | | | | 1)2106 | | | | | | 1)0706 | |
| | | | | | 1)6f348 | | | | | | 1)69?5 | |
| 1177fj | 38973 | 42537 | ,B897 | 5302, | 6017f; | 67739 | 70369 | 65506 | 65H3 | 65H3 | 70185 | 68319 |

2.2 Raw data of anti-replicon activity assay

1a plate 1
coiumcolum colum colum colum coium colum colum colum colum coium colum

| | | | | | | | | | | | |
|---|---|---|---|---|---|---|---|---|---|---|---|
| 8 | 732 | 3758 | 3795 | 4068 | 4308 | 3768 | 3932 | 3632 | 3,108 | 3540 | 3592 |
| 24 | 10GO | 3388 | 417f3 | 3104 | 3f372 | 38′:16 | 3340 | 3132 | 3468 | 3248 | 3236 |
| 28 | 3″172 | 39″i6 | 4364 | 415G | 3f3GO | 3384 | 3312 | 35H3 | 3380 | 3336 | 3G84 |
| 32 | 373l) | 4300 | 4028 | 4428 | 3840 | 3904 | 36f38 | 3828 | 3852 | 3812 | 3804 |
| 20 | 2120 | 4036 | | 4452 | 4276 | 3728 | 3708 | 4092 | 3676 | 3656 | 4148 |
| 28 | 2040 | 4080 | | 4·!56 | ,13″16 | 4084 | 4008 | 3fJ12 | 3992 | | 3844 |

1a plate 2
coiumcolum colum colum colum coium colum colum colum colum coium colum

| | | | | | | | | | | | |
|---|---|---|---|---|---|---|---|---|---|---|---|
| | 3312 | 41G8 | 3624 | 4348 | 3636 | 3592 | 3756 | 3188 | | 3488 | 3396 |
| 28 | 3552 | 4188 | 3480 | 4268 | 3f312 | 3580 | 3592 | 3832 | 3748 | 3384 | 33′:16 |
| 28 | 379 | 4396 | 47f3 | 4S04 | :f7t18 | 429 | 3688 | 3452 | | 3f300 | 3720 |
| 20 | 4112 | 728 | J508 | 2804 | 3524 | 40.12 | 4076 | | 3760 | 3856 | 4032 |
| 12 | 52 | 6 | | 1088 | 2800 | 3880 | 4000 | 4284 | 4360 | 3912 | 4188 |
| 24 | 341f3 | | 3304 | 3688 | 3620 | 3400 | 3400 | 3348 | 3048 | 309G | 3388 |
| 28 | 3464 | 3236 | 3852 | | 3400 | 3760 | 3316 | 321fj | 3048 | 3020 | 3338 |
| 24 | 2968 | 3176 | 347f; | | 3324 | 3440 | 3196 | 2748 | 2628 | 3108 | 3524 |
| 40 | 3″180 | 2932 | 3408 | 2956 | 3696 | 3264 | 2912 | 3480 | 2768 | 2776 | 3596 |
| 28 | 3″132 | 3760 | 3P32 | 3175 | 3548 | 3452 | 39f38 | 3172 | 319G | 3228 | 3740 |
| 20 | 3248 | 397t) | 3888 | 3724 | 40t10 | 3484 | 3440 | 3328 | 3028 | 309G | 3496 |
| 20 | 3?88 | 38S2 | 3f3t14 | 3728 | 3944 | 84 | 3436 | 3192 | 3348 | | 3′>88 |
| 36 | 3548 | 3964 | 341fj | 3352 | 3280 | 3232 | 3188 | 3200 | 3052 | | 3576 |
| 32 | 3856 | 3876 | 4044 | | 3364 | 3876 | 3600 | 3080 | | 3356 | 3524 |
| 24 | 4048 | 4036 | 3980 | 3124 | | 3704 | 3780 | 3388 | 3312 | 3504 | 3880 |
| 24 | 172 | 1180 | | 3318 | 3591 | 3591 | 3820 | 3208 | 3024 | | 4340 |
| 16 | 32 | 232 | 752 | 2216 | 3372 | 3668 | 4032 | 4116 | 3852 | 4208 | 4095 |

row H
2a plate 2

| | | | | | | | | | | | |
|---|---|---|---|---|---|---|---|---|---|---|---|
| 2,1 | 2844 | 2950 | 2856 | 2,112 | 25,14 | 2548 | 2388 | 2388 | 2304 | 2564 | 2352 |
| 32 | 3′172 | 2856 | 2708 | 2652 | 2388 | 2200 | 2428 | 205f3 | 2444 | 2328 | 2224 |
| 32 | 2″136 | 2504 | 2360 | 2268 | 2108 | 2156 | 2248 | 209f3 | 2304 | 2056 | 24′:12 |
| 20 | 2280 | 2720 | 2l)84 | 2260 | 2332 | 2244 | !304 | 2572 | 2208 | 1888 | 2S32 |
| 28 | 3068 | 2664 | 2908 | 2524 | 2804 | 3092 | 2484 | 2f;08 | 2380 | 2232 | 241fj |
| 15 | 2820 | 2984 | 3016 | 28fJ2 | 2944 | 2955 | 2804 | 2392 | 2752 | 2628 | 32.15 |

row ″\.J
row H
2a plate2t

| | | | | | | | | | | | |
|---|---|---|---|---|---|---|---|---|---|---|---|
| 20 | 2700 | 2812 | 2628 | 2572 | 2524 | 2504 | 2450 | 2450 | 2,184 | 2456 | 2596 |
| 20 | 2700 | 2812 | 2628 | 2572 | 2524 | 2504 | 2450 | 2450 | 2,184 | 2456 | 2596 |
| 28 | 2752 | 2768 | 24H3 | 2208 | 2804 | 2440 | 2188 | 2884 | 2204 | 2240 | 2548 |
| 24 | 2508 | 30·H.1 | 2S68 | 2S80 | 2′744 | 20 [symbol] .14 | 504 | 2288 | 2084 | 21( )8 | 2S04 |
| 36 | 2676 | 2740 | 2740 | 2404 | 2536 | 2632 | 2236 | 2016 | 2408 | 2228 | 2232 |
| 28 | 56 | 184 | 548 | 1024 | 1428 | 2435 | 2″.<′,.- | 28 | 56 | 184 | 548 |
| | | | | | | | 1,_0 | | | | |
| 20 | ,18 | 200 | 588 | 13fJ6 | 1856 | 2248 | 2712 | 2532 | 2284 | 2520 | 2820 |

3 Cytotoxicity and anti-replicon activity of the human plasma derived proteins. CC:;o and EC50 values are summarized in Table 4. GraphPad Prism files containing dose-dependent curves are presented in this report. CC50 and EC50 values are shown in FIG. 1 and FIG. 2 respectively.

TABLE 4
CC50 and EC50 Summary of the human plasma derived proteins
Ribavirin

| | | | | | 1c | |
|---|---|---|---|---|---|---|
| | 1a | | 1b | | | EC50 |
| Name | CC50 (ug/ml) | EC50 (ug/ml) | CC50 (ug/ml) | EC50 (ug/ml) | CC50 (ug/ml) | (ug/ml) |
| AFOD | 60.7% @ | 76.5% @ | >400 | >400 | >400 | >400 |
| KH | 400 ug/ml | 400 ug/ml | | | | |
| AFCC | >400 | >400 | >400 | >400 | >400 | >400 |
| KH | | | | | | |
| AFCC | 33.8% @ | 44.5% @ | >400 | >400 | >400 | >400 |
| RAAS 1 | 400 ug/ml | 400 ug/ml | | | | |
| AFCC | >400 | >400 | >400 | >400 | >400 | >400 |
| RAAS 4 | | | | | | |
| AFCC | >400 | >400 | >400 | >400 | >400 | >400 |
| RDNA | | | | | | |
| | CC50 (uM) | EC50 (uM) | CC50 (uM) | EC50 (uM) | CC50 (uM) | EC50 |
| | | | | | | (uM) |

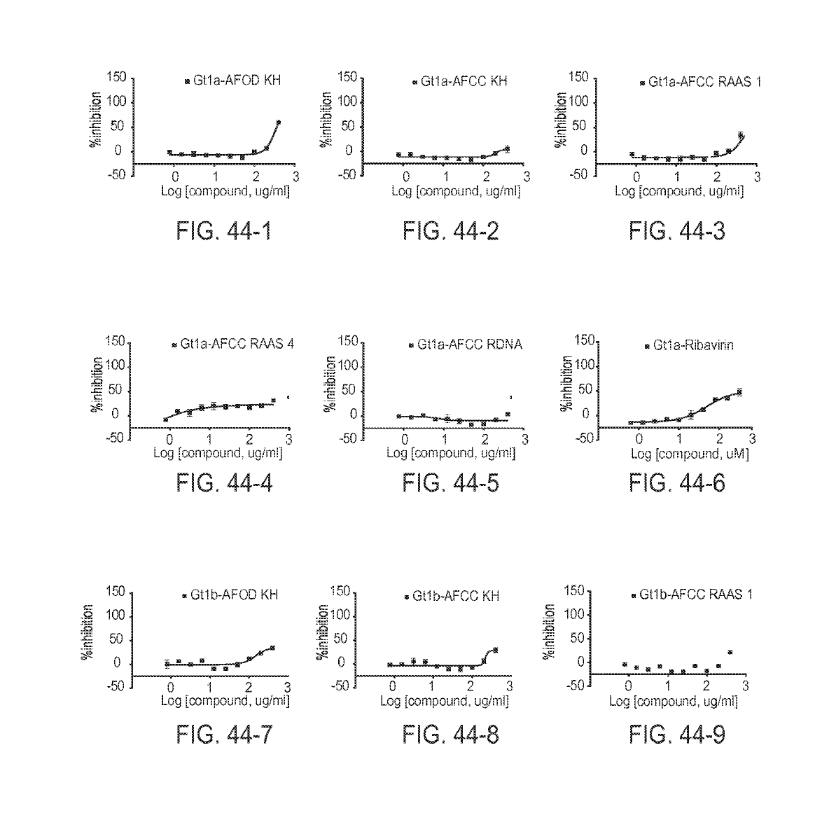

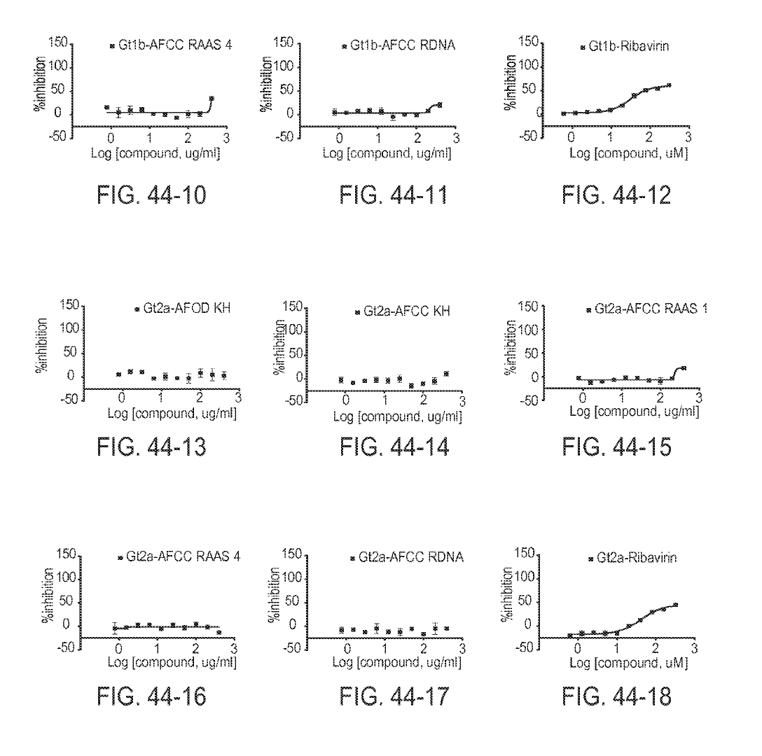

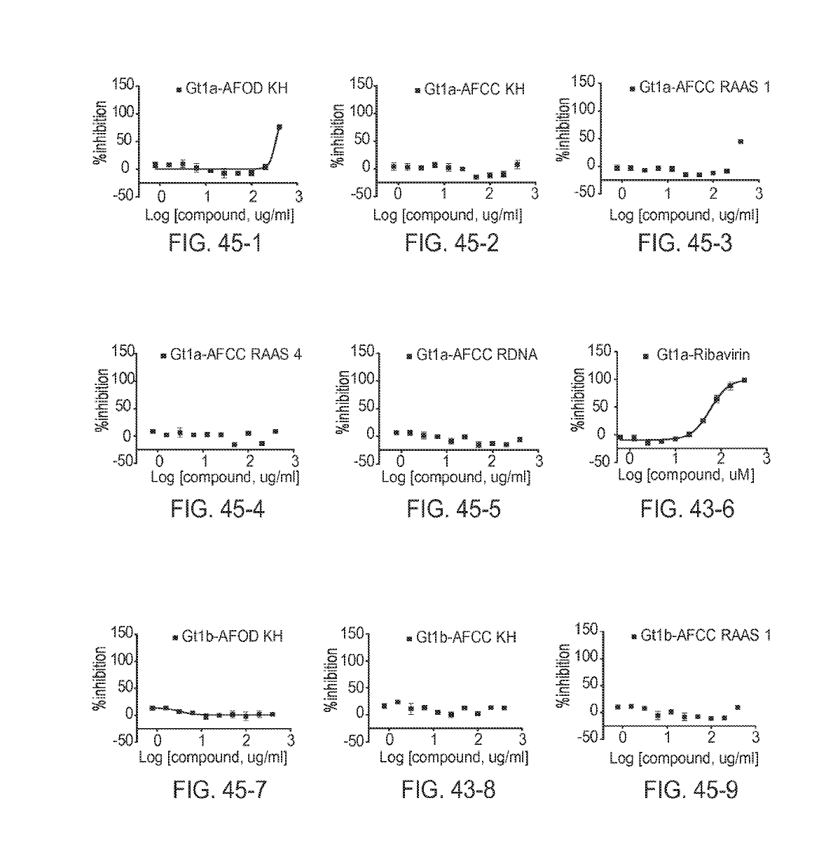

FIGS. 44-1 through 44-18. Dose-dependent curves (CC 50 values)

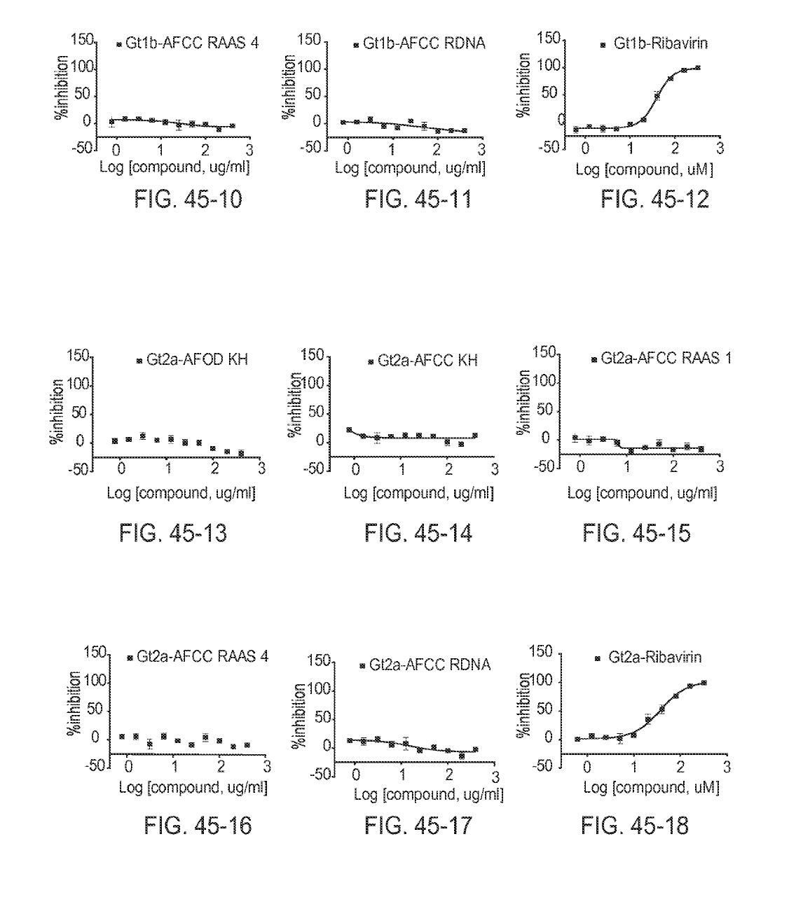

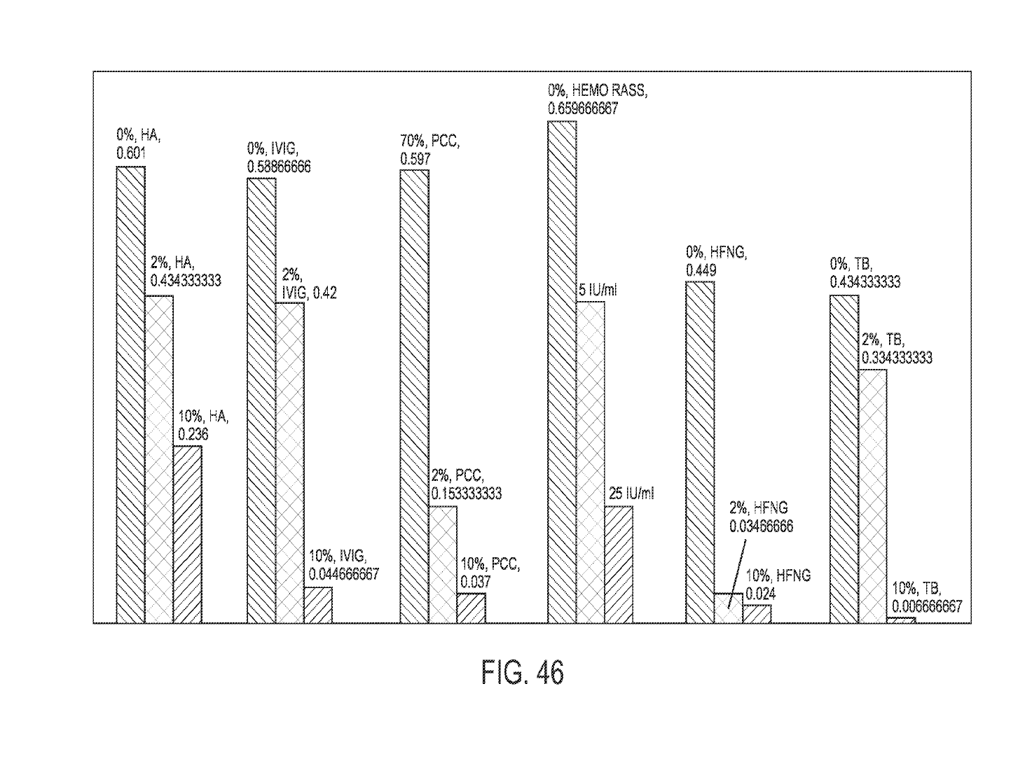

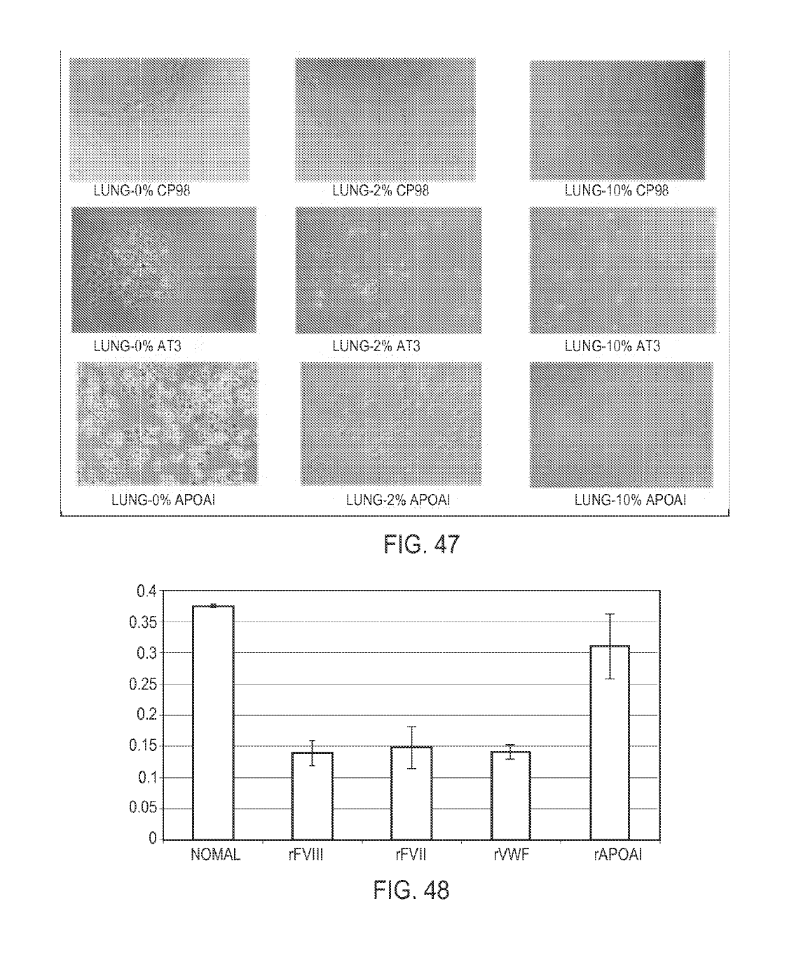

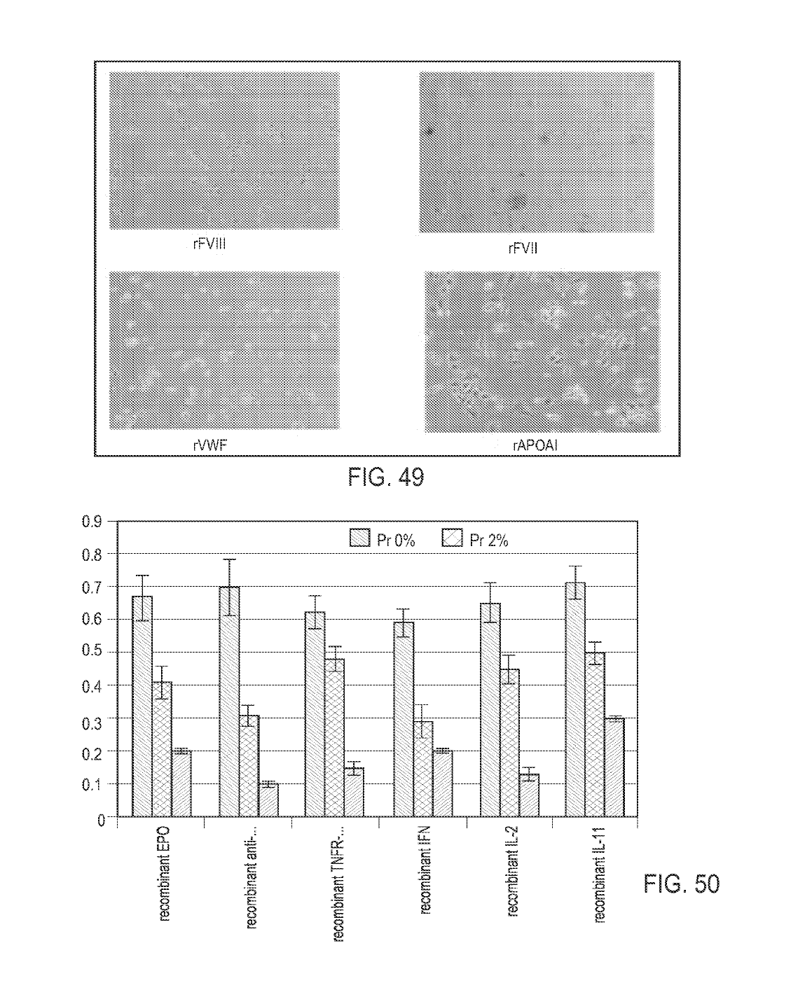

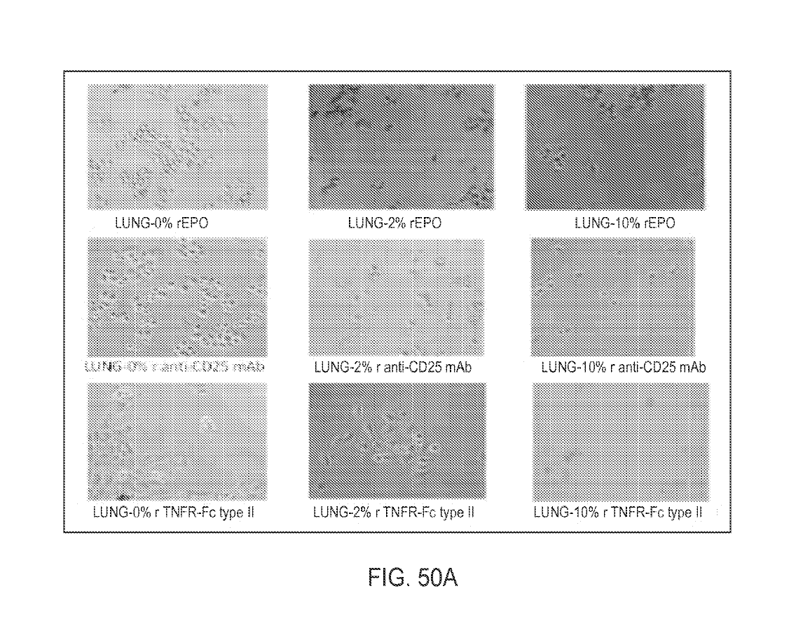

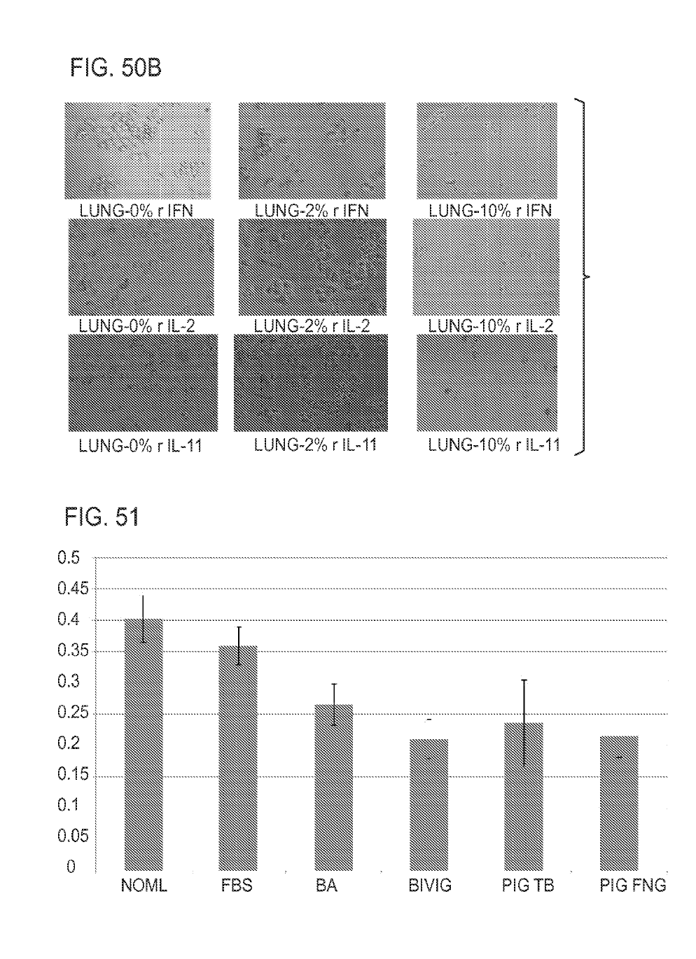

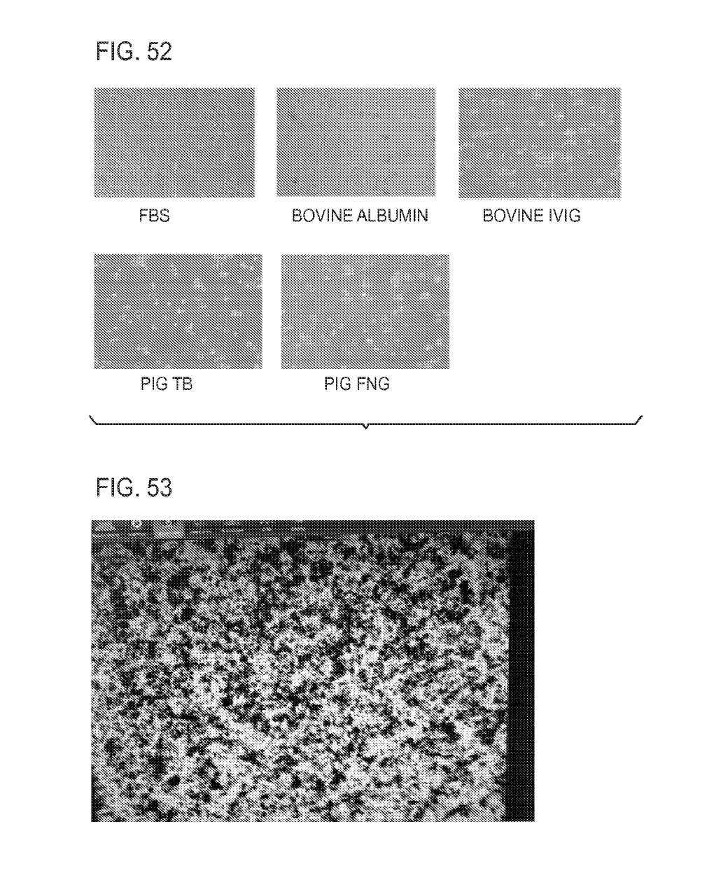

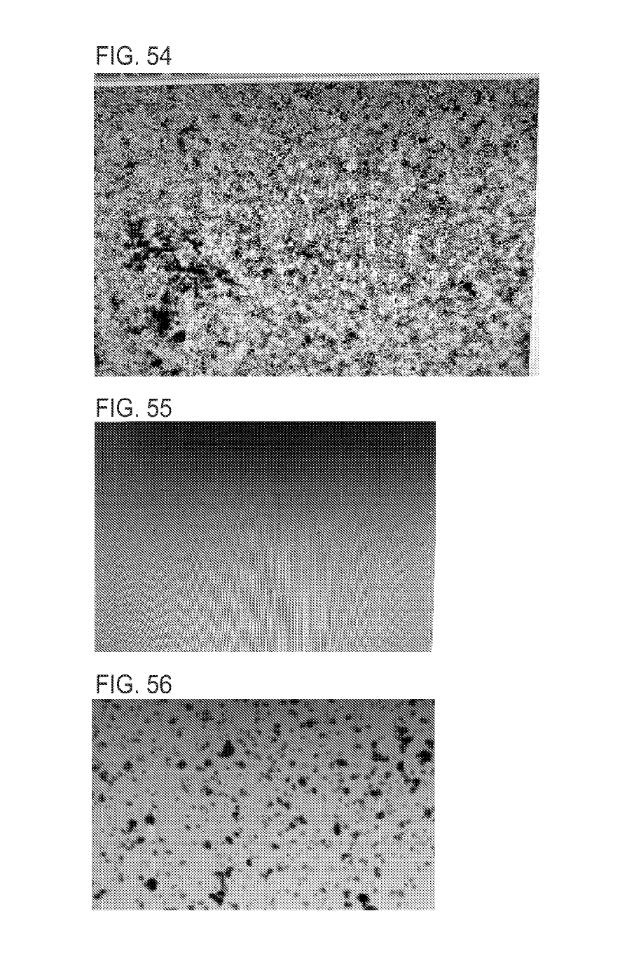

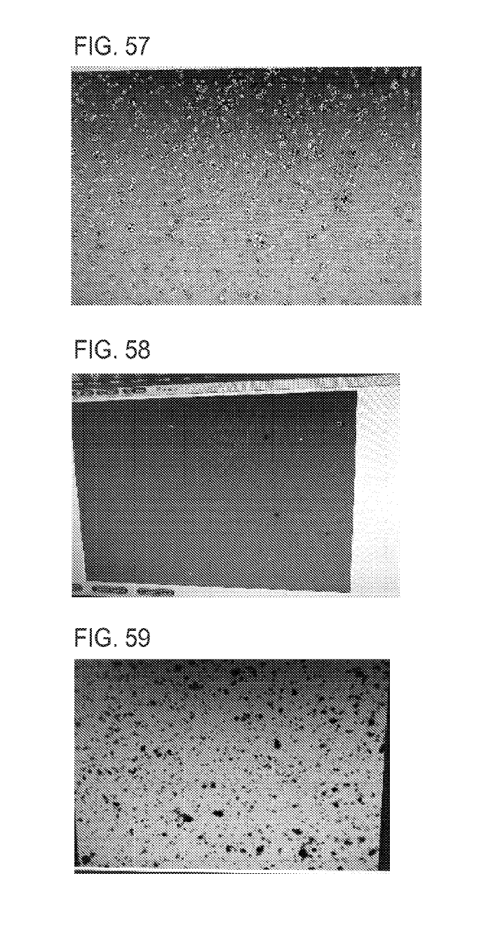

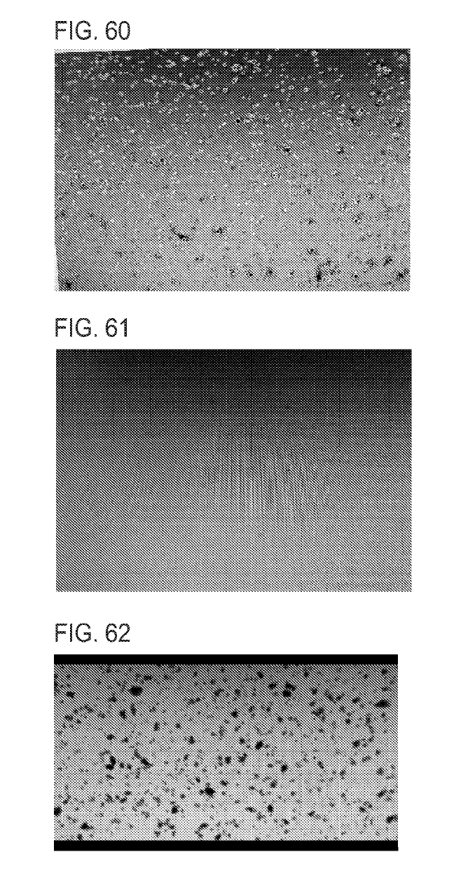

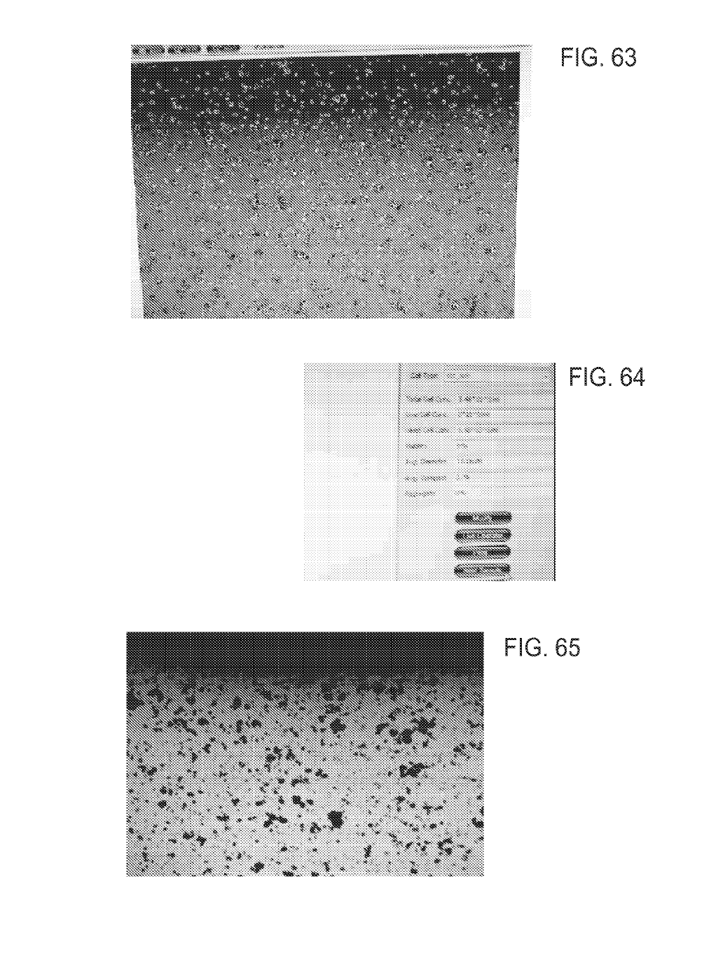

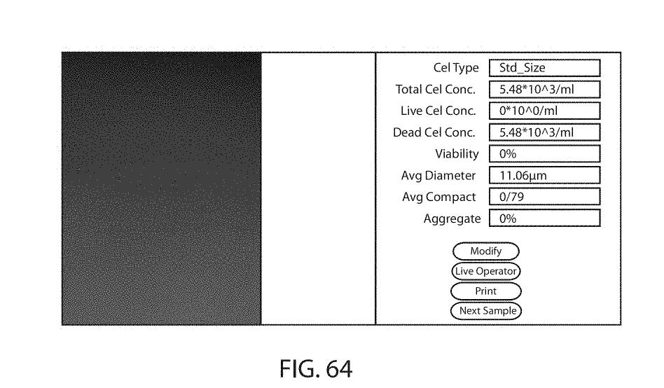

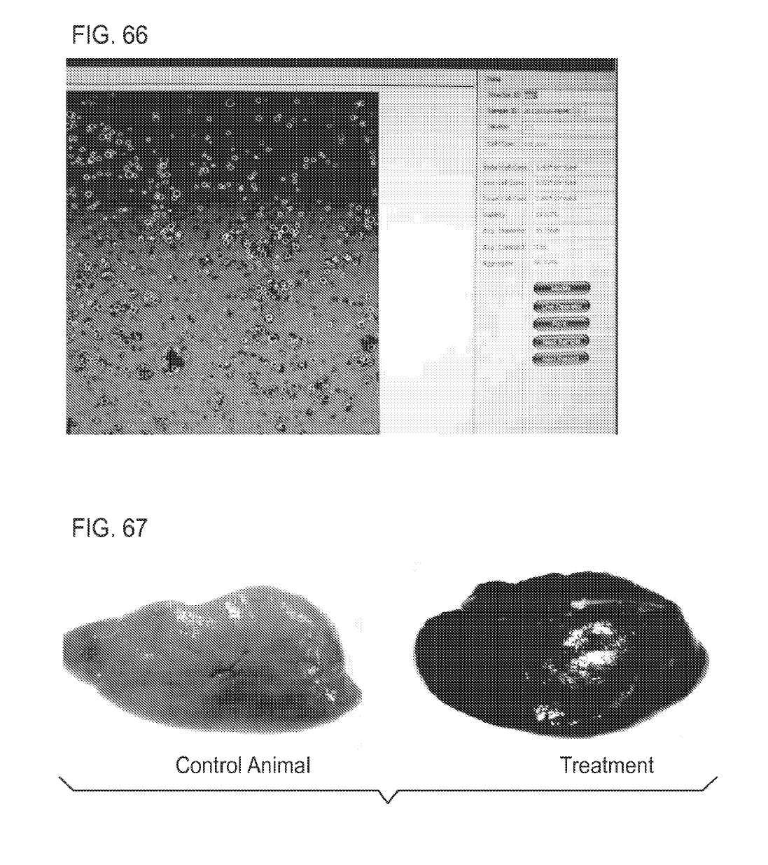

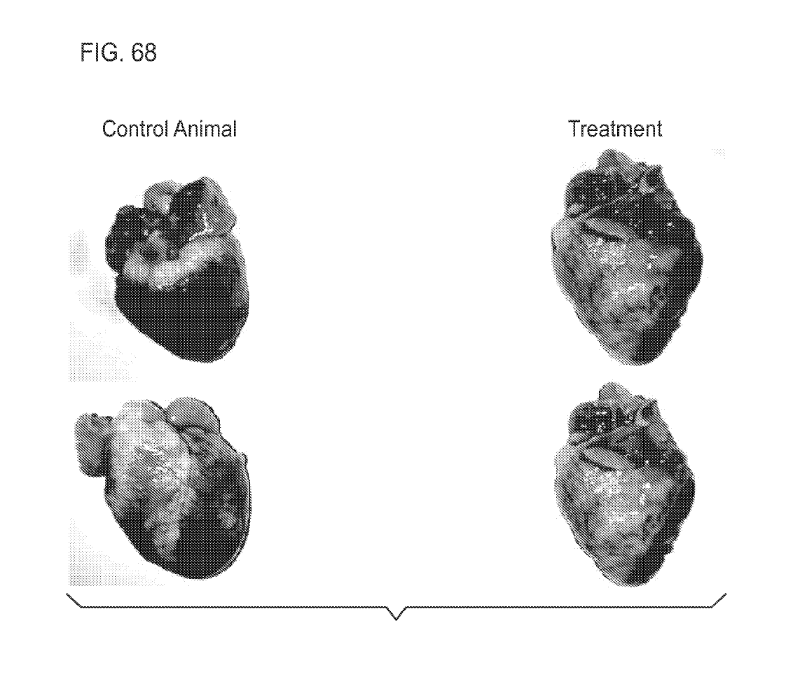

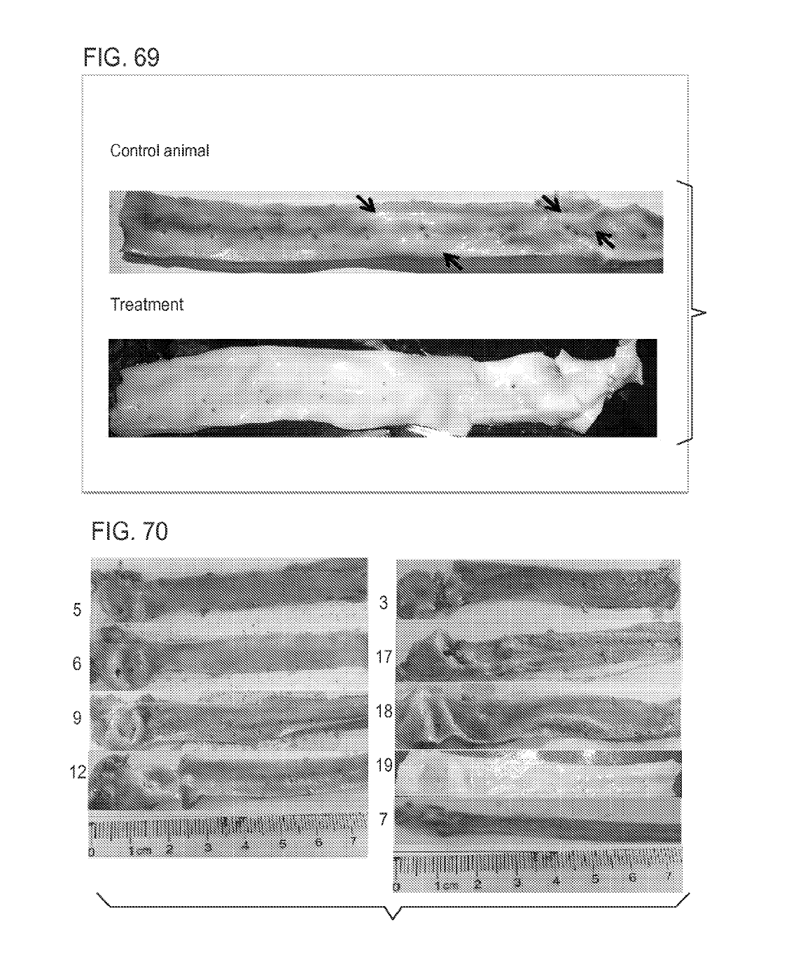

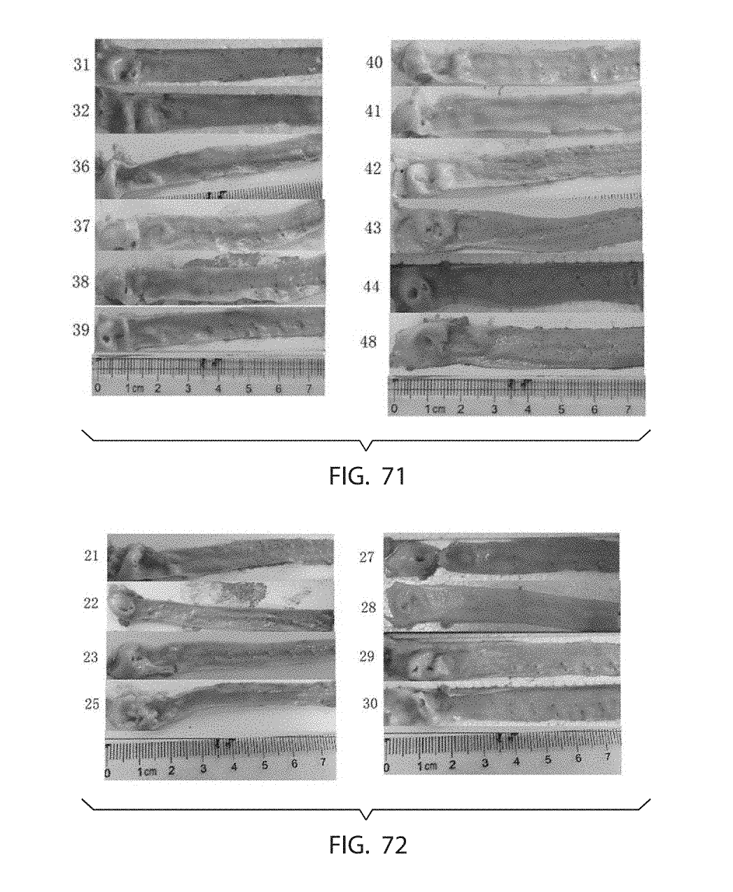

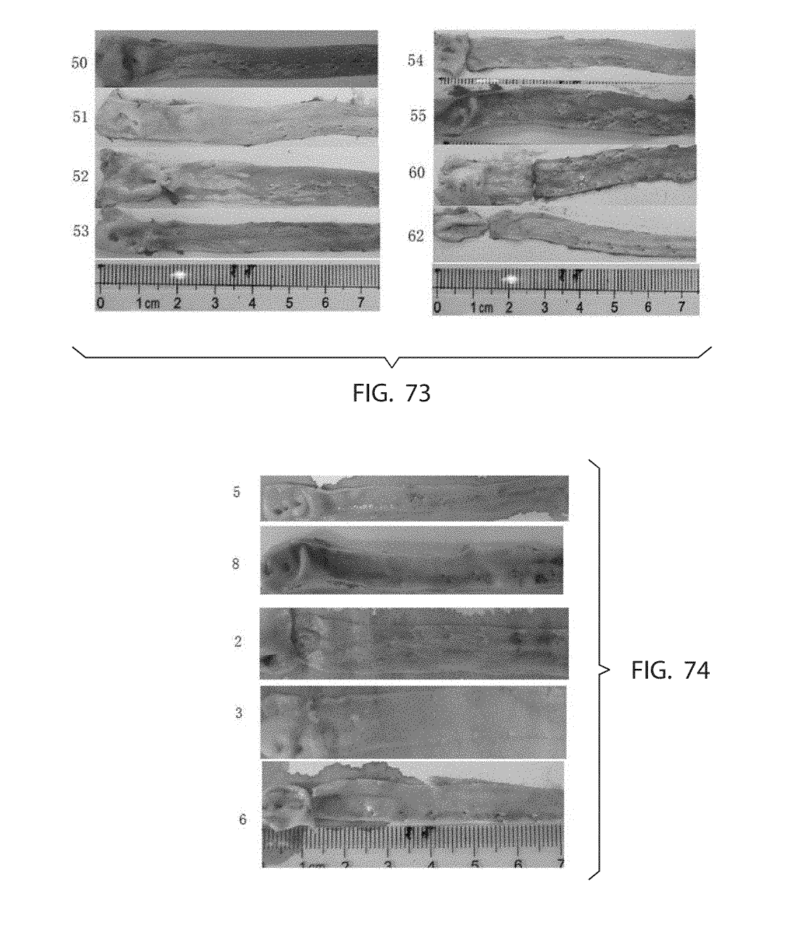

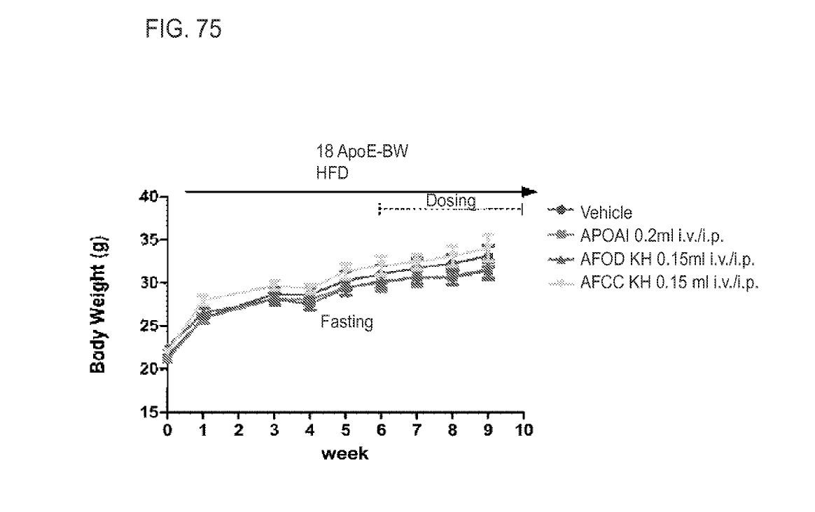

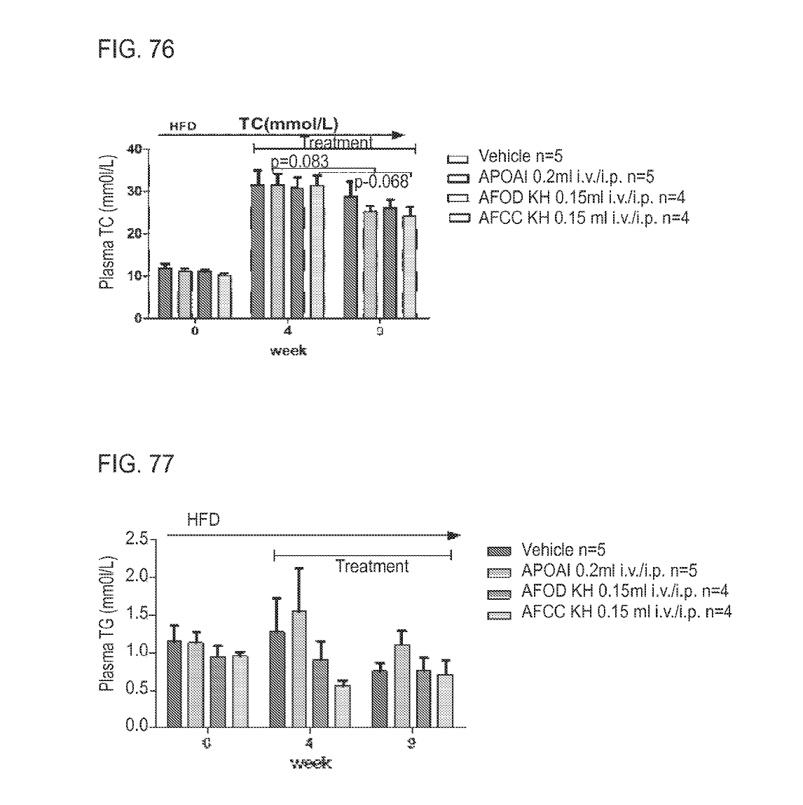

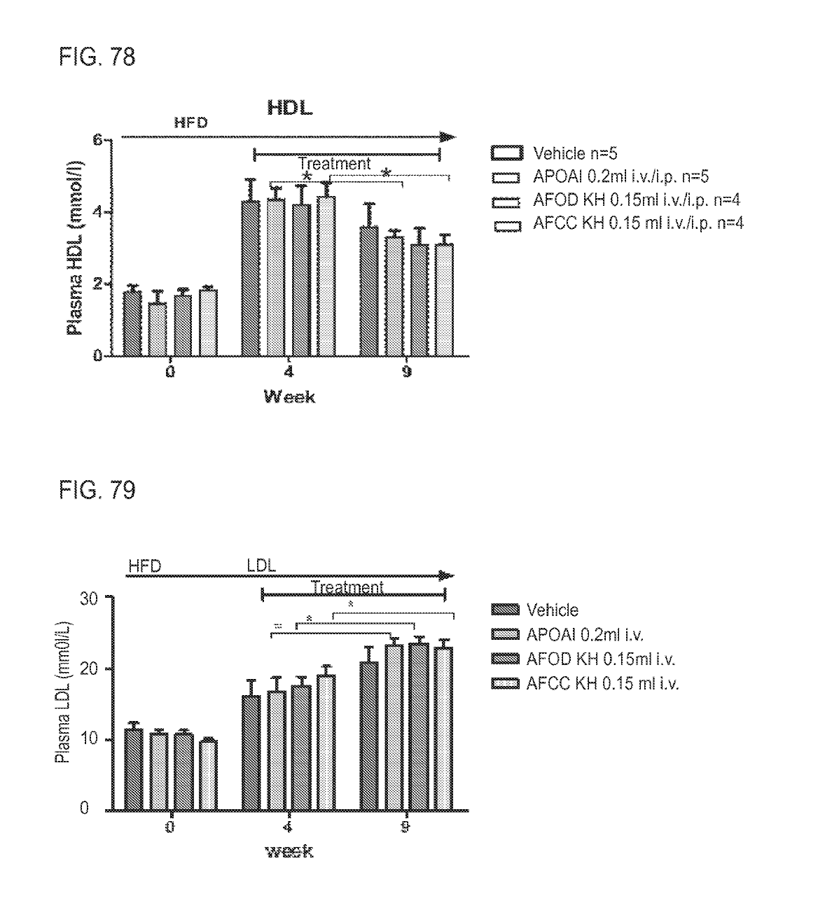

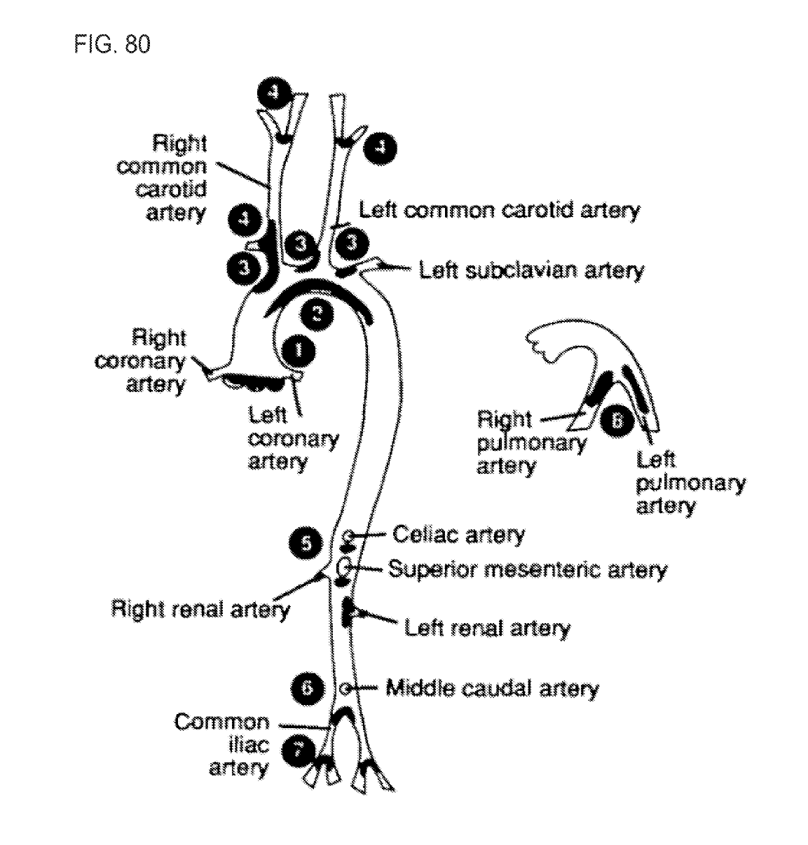

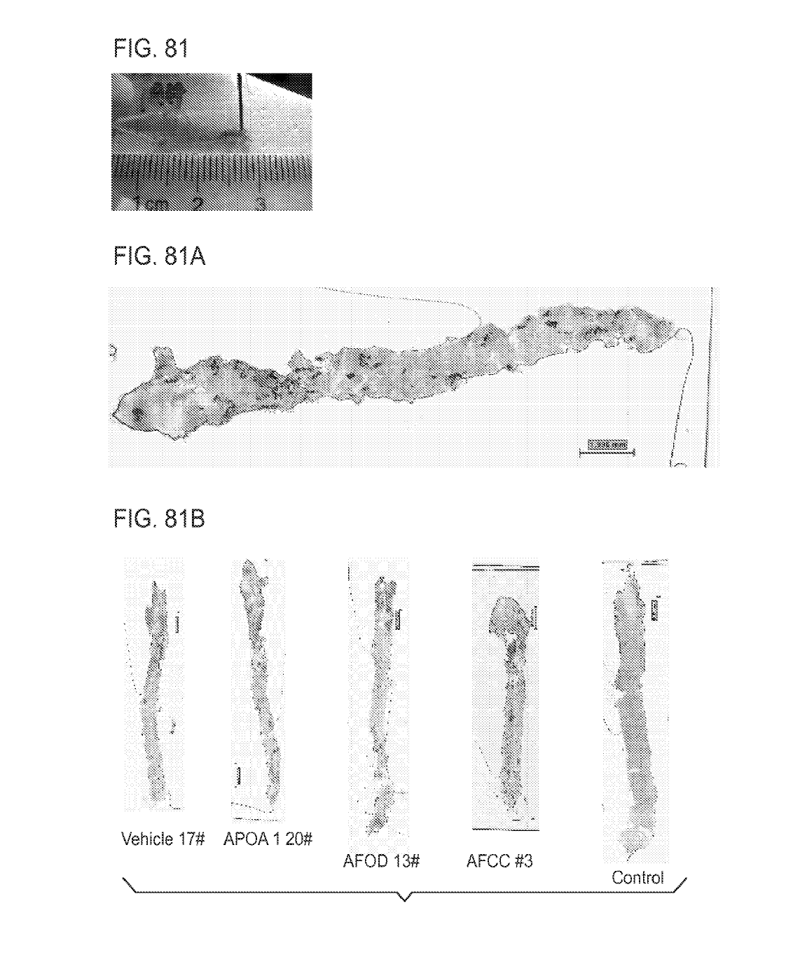

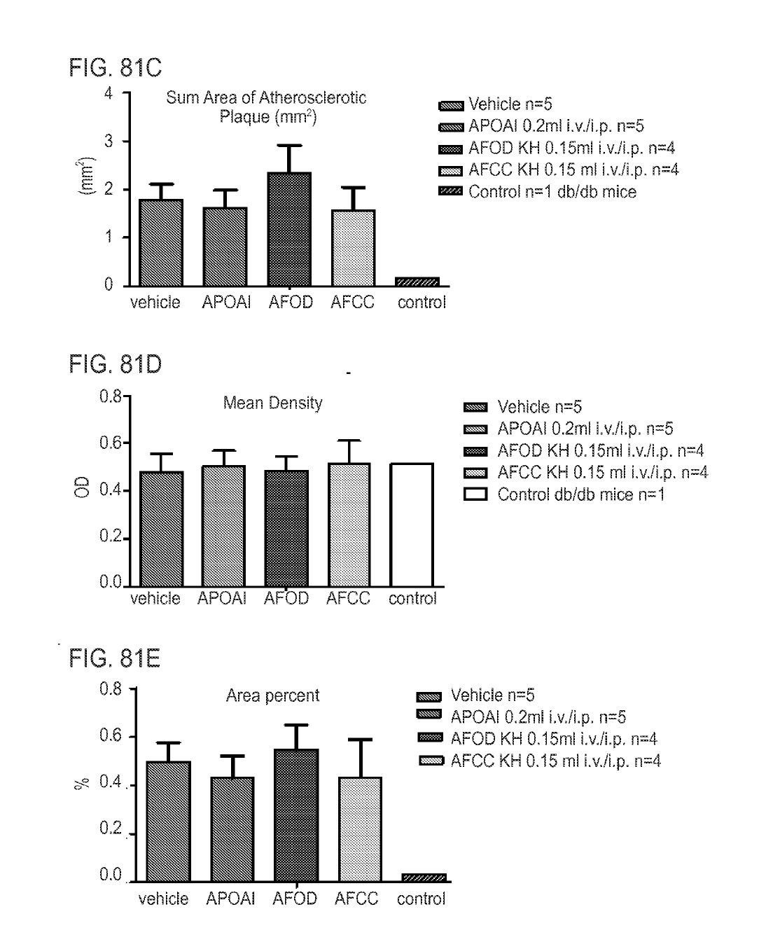

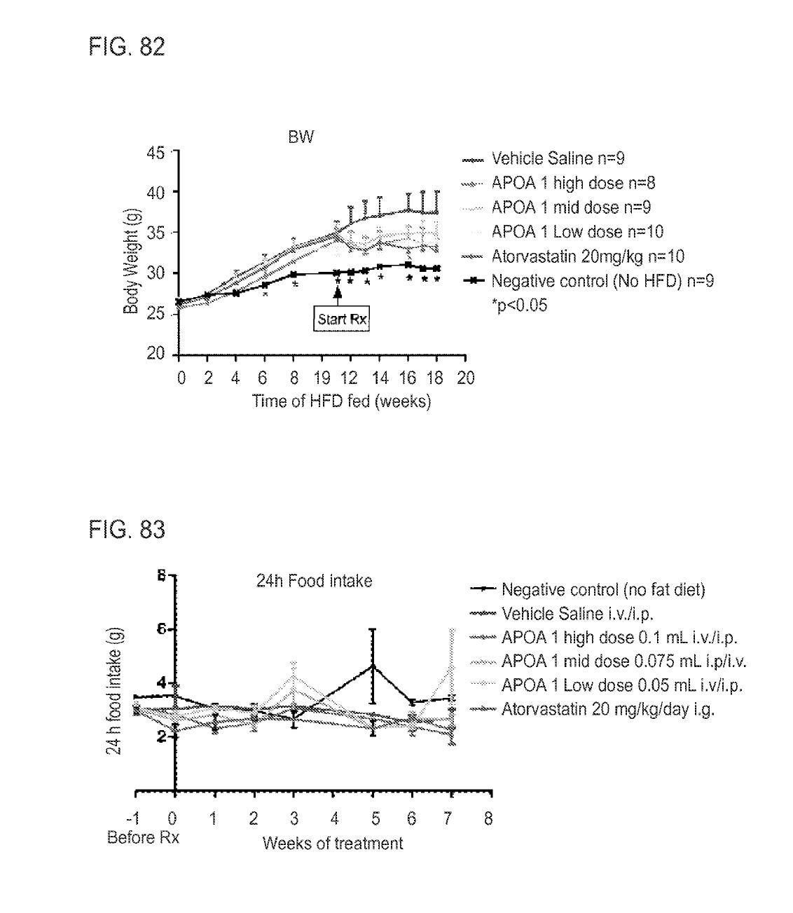

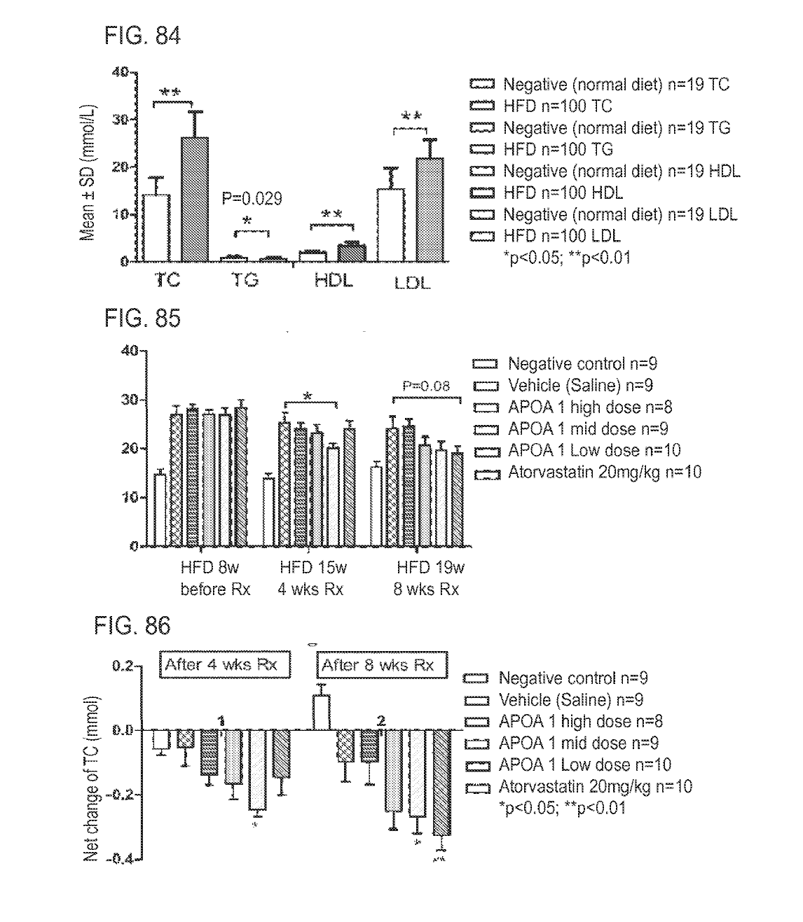

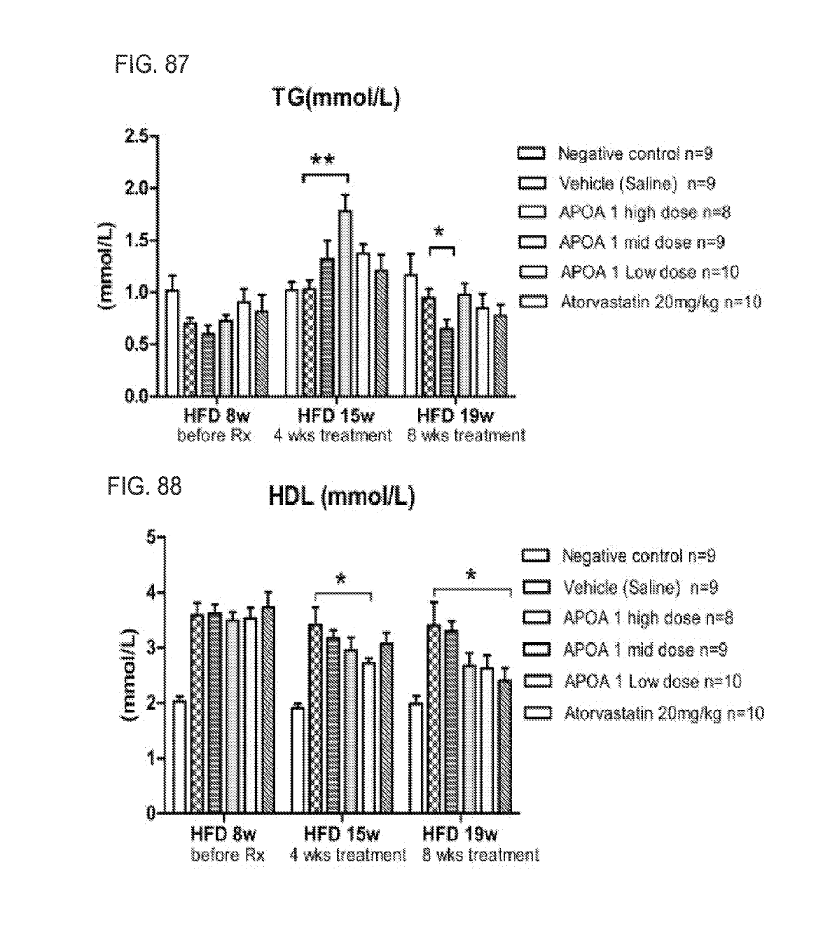

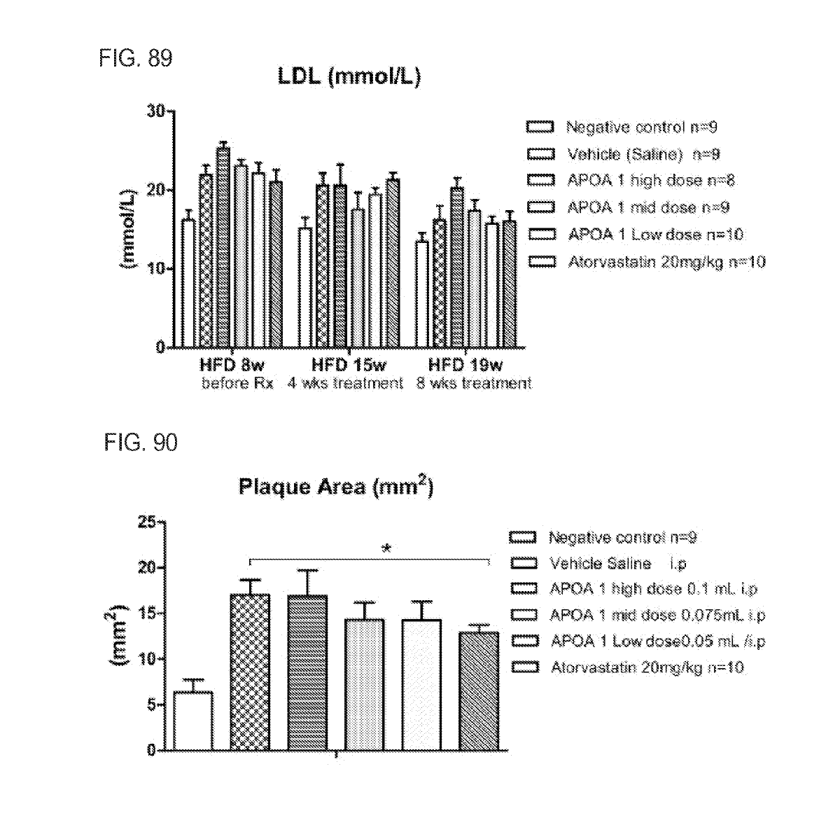

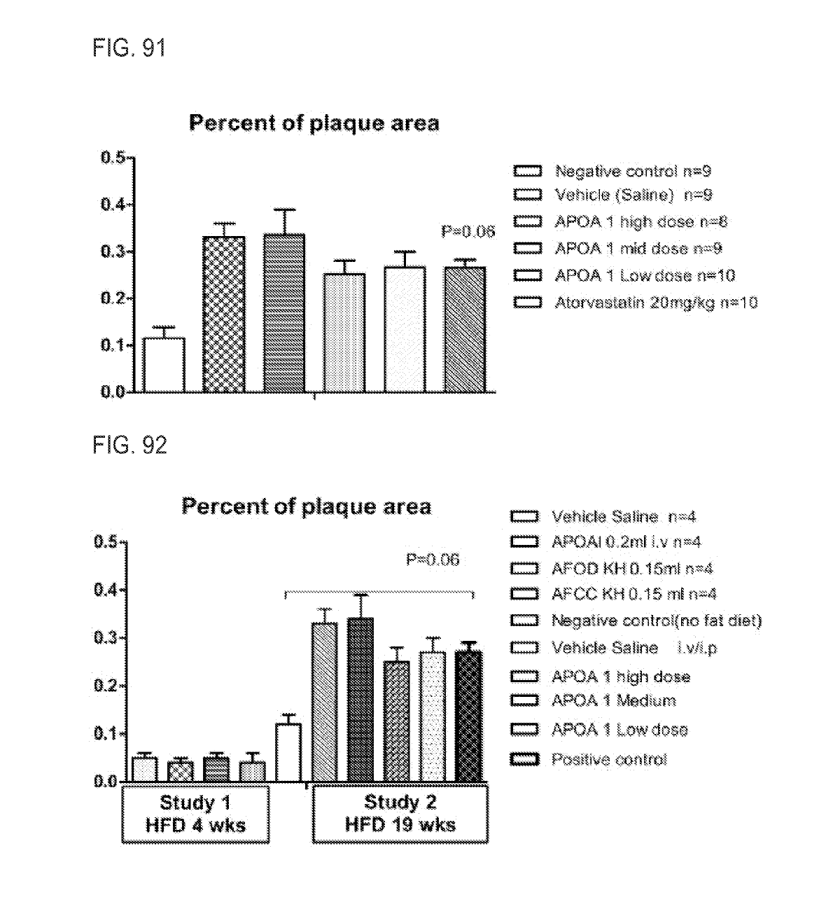

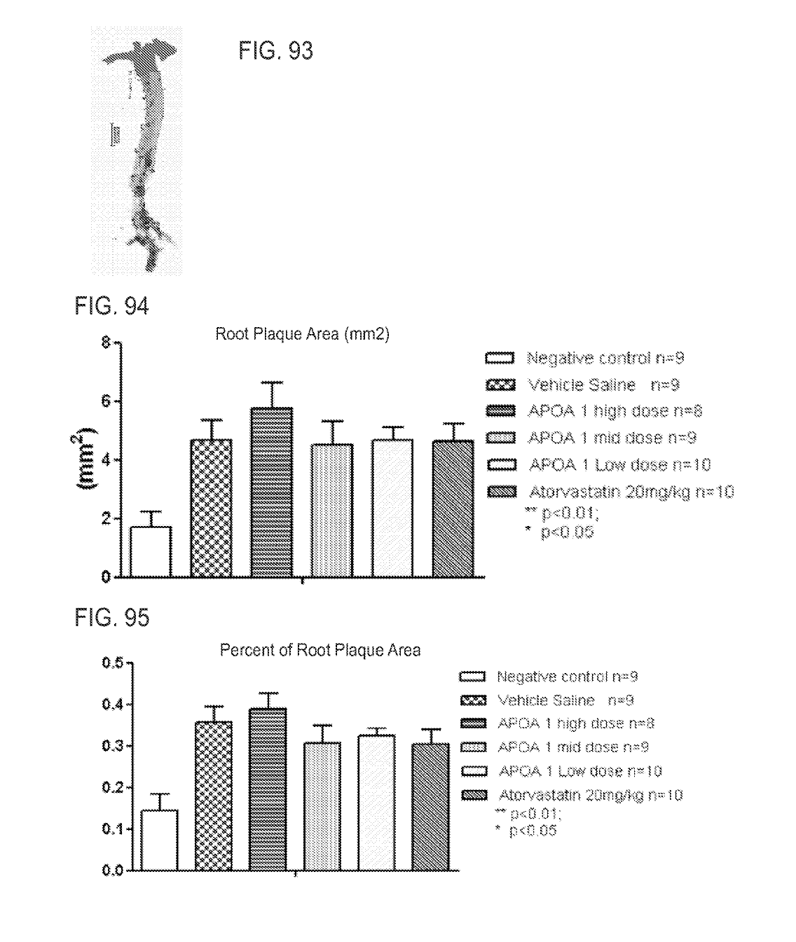

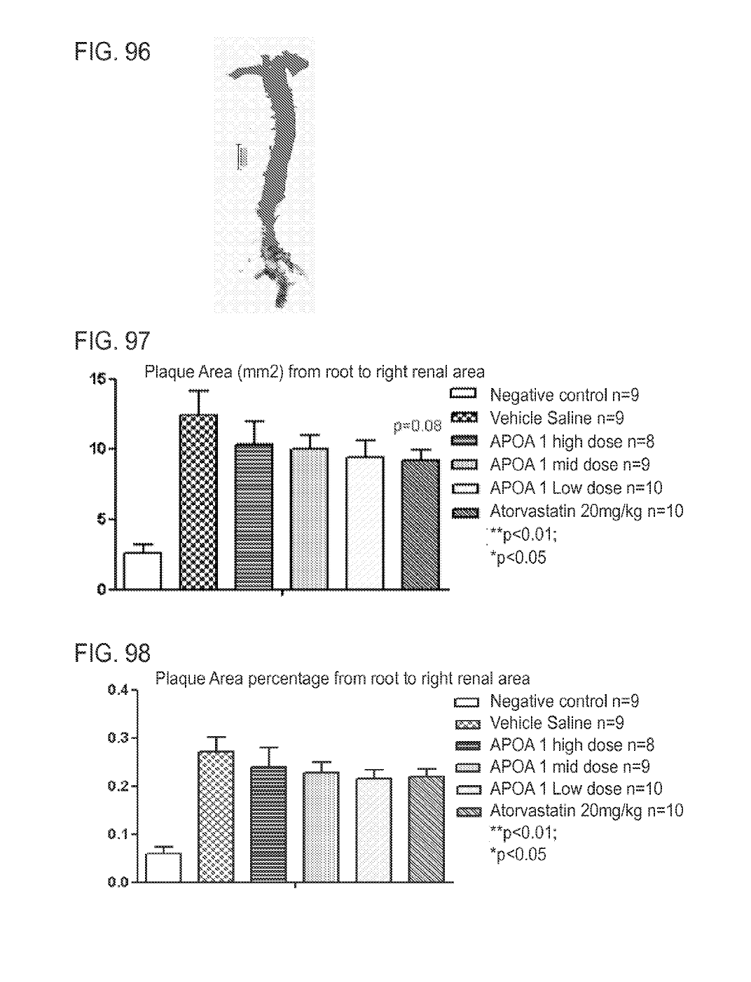

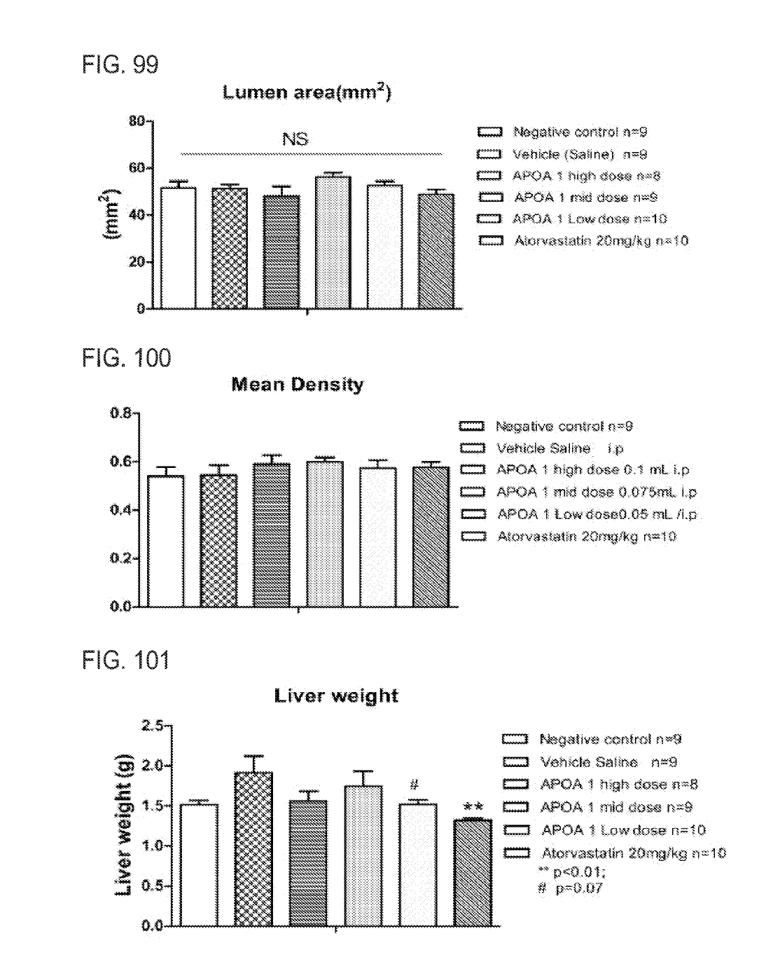

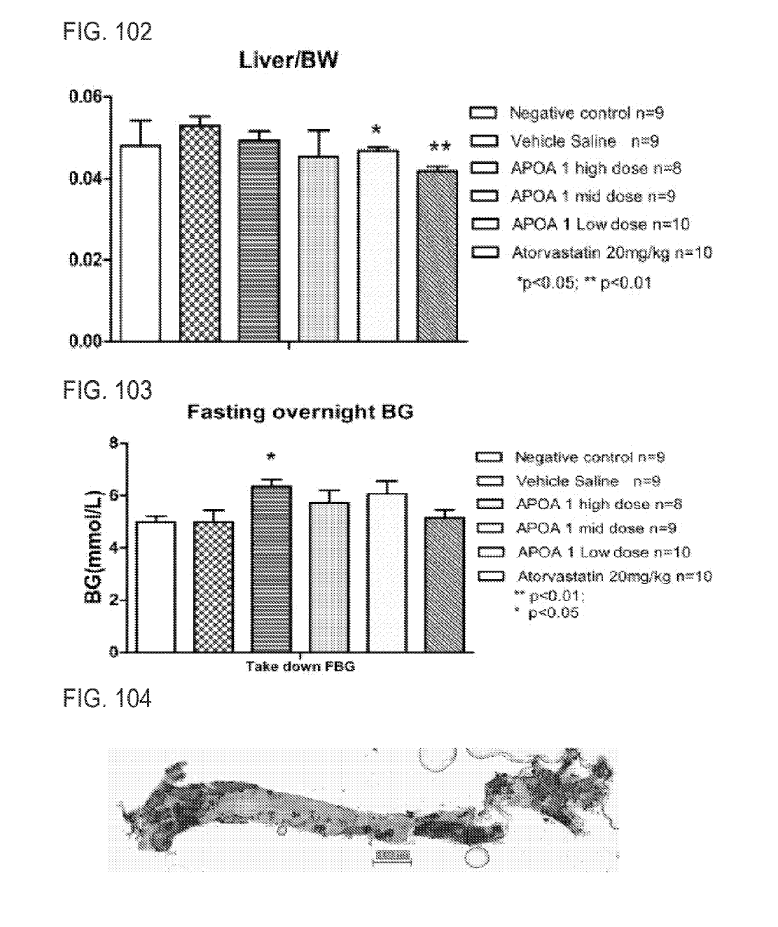

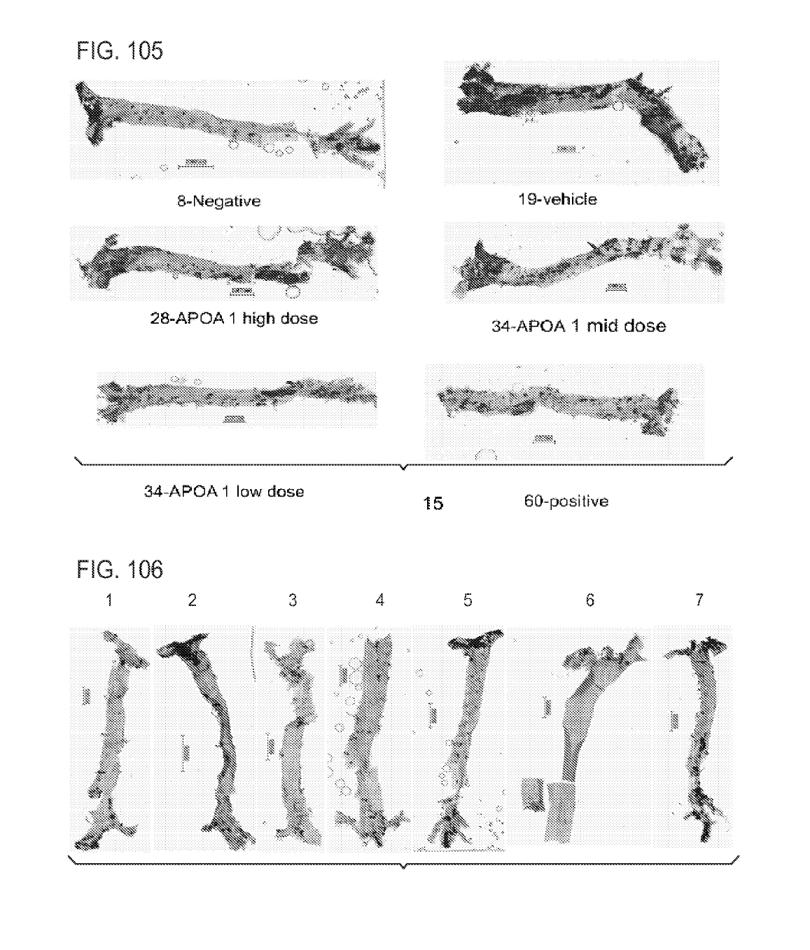

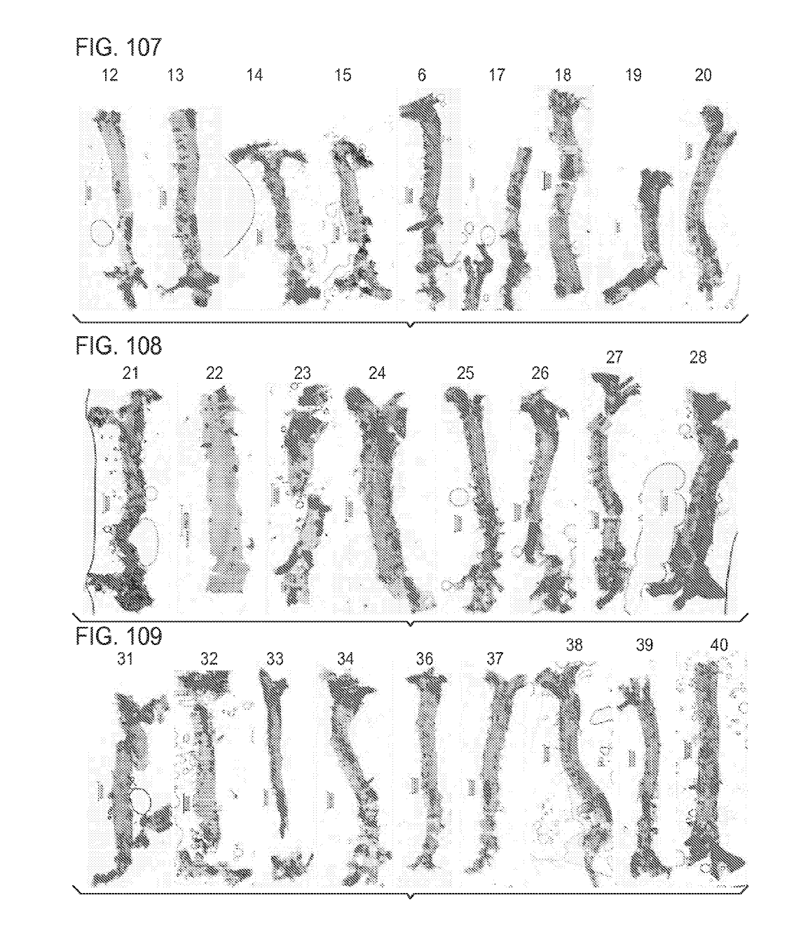

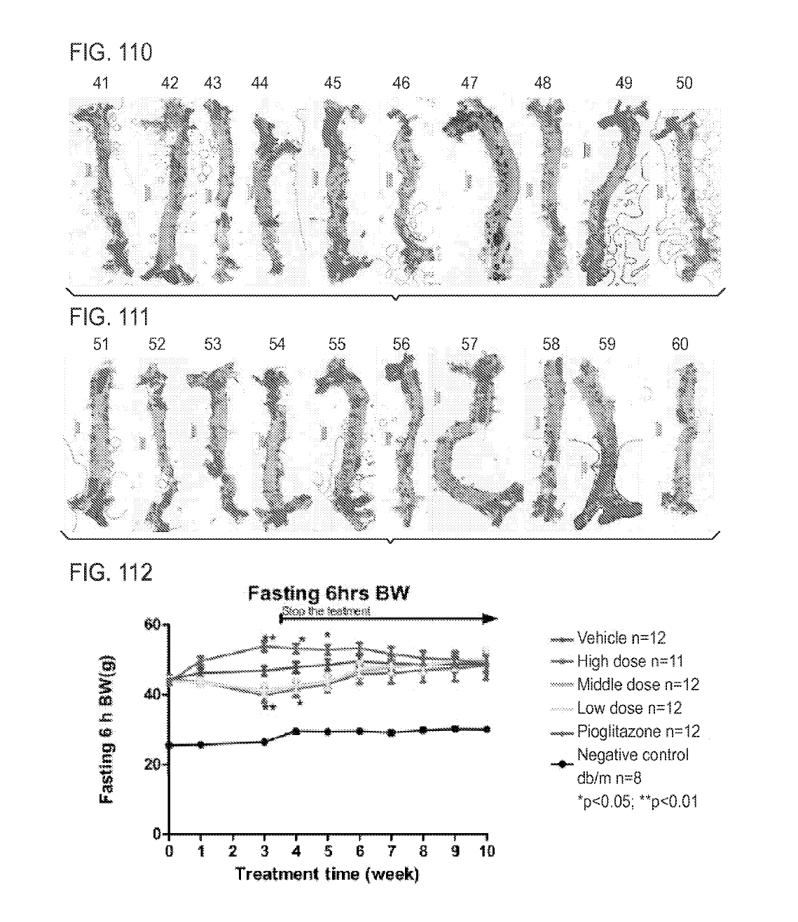

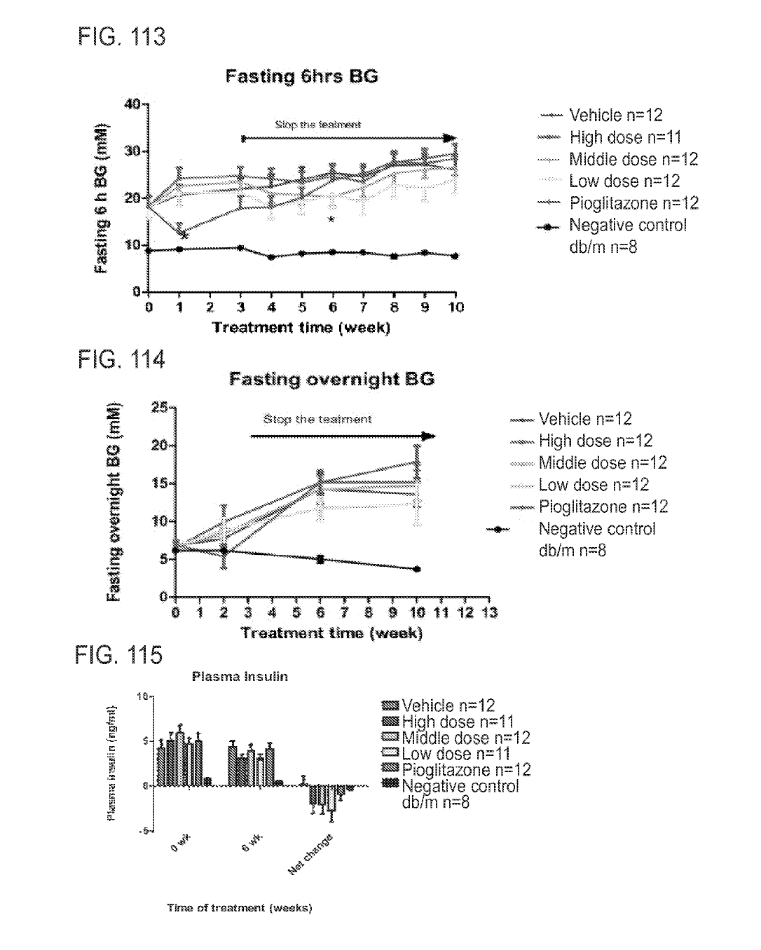

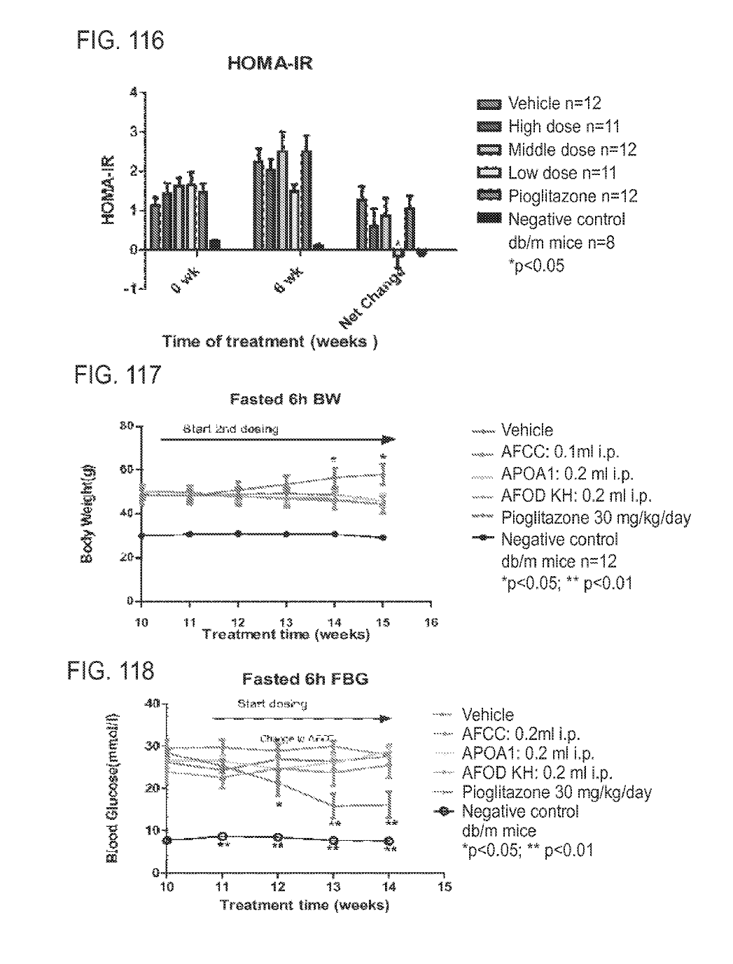

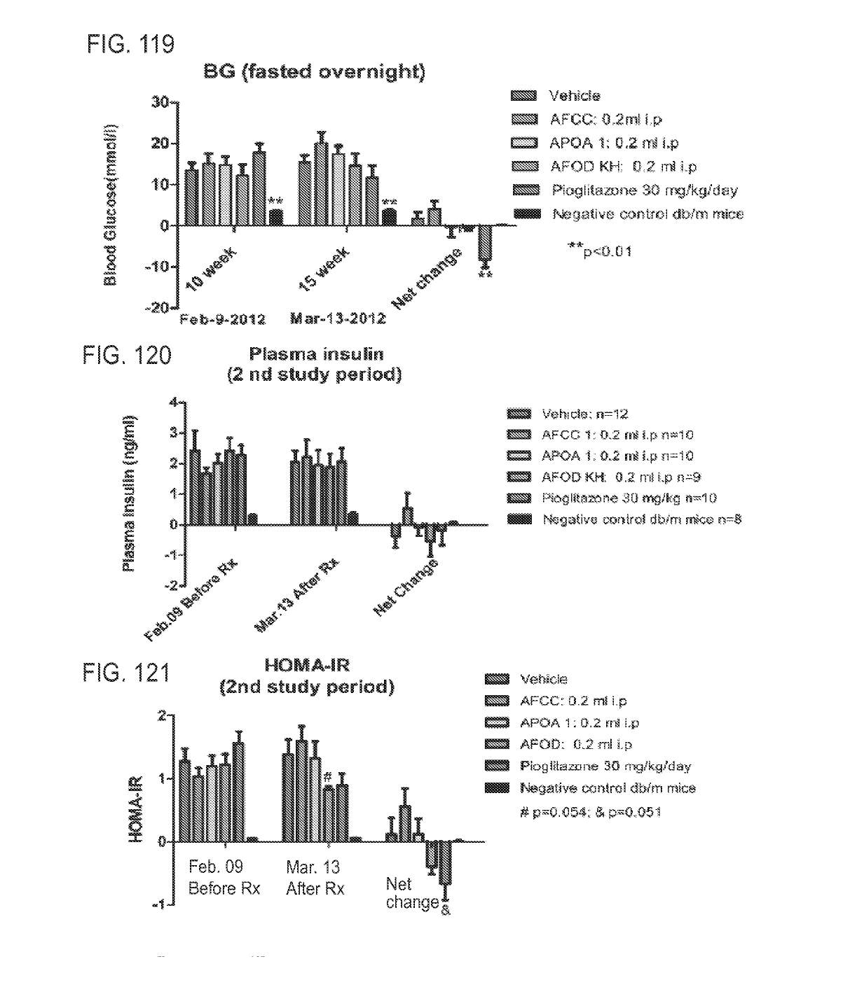

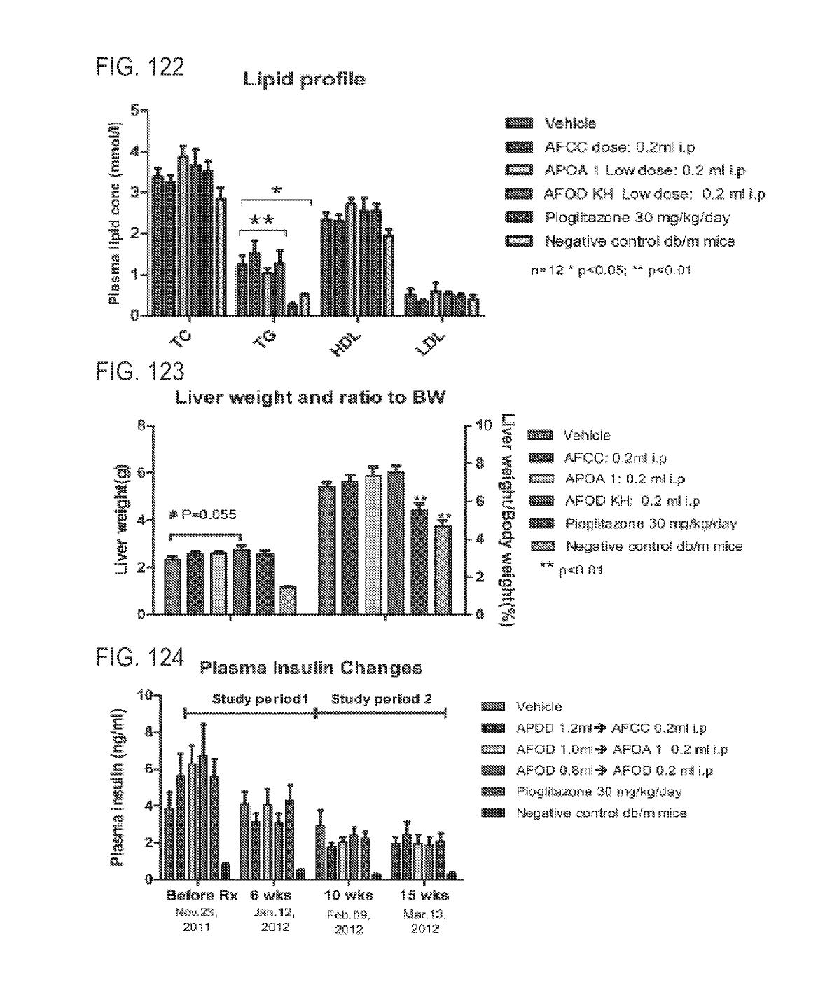

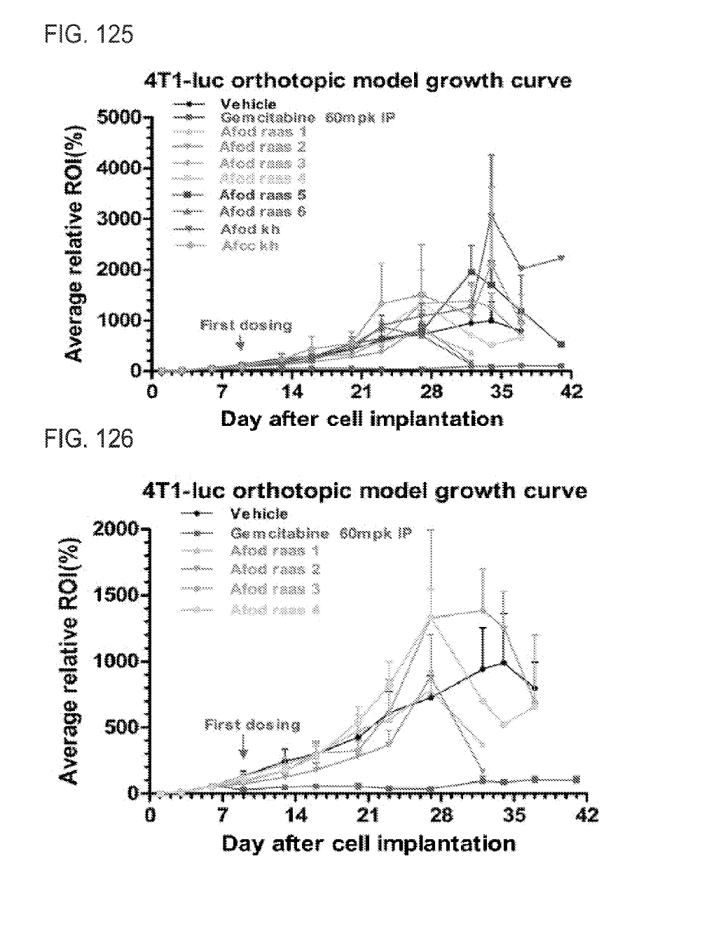

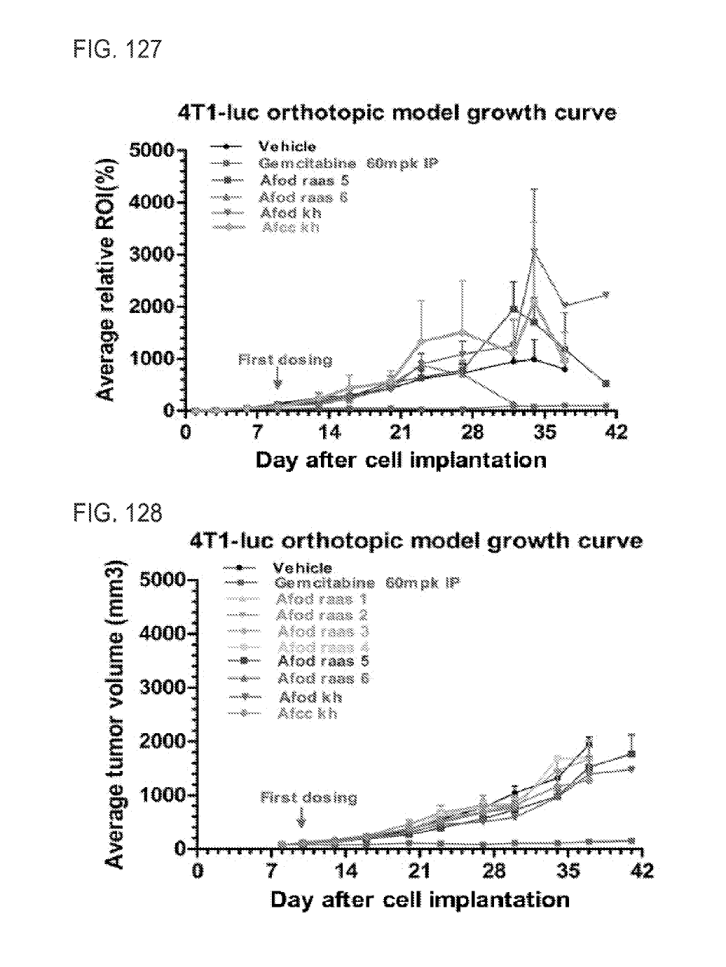

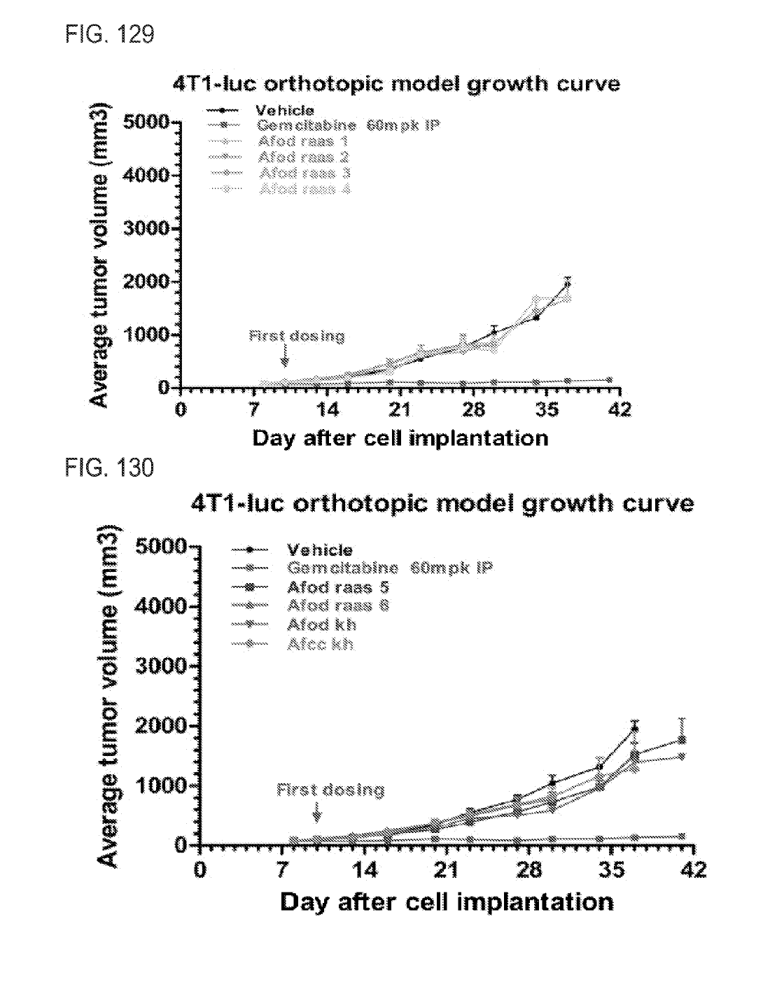

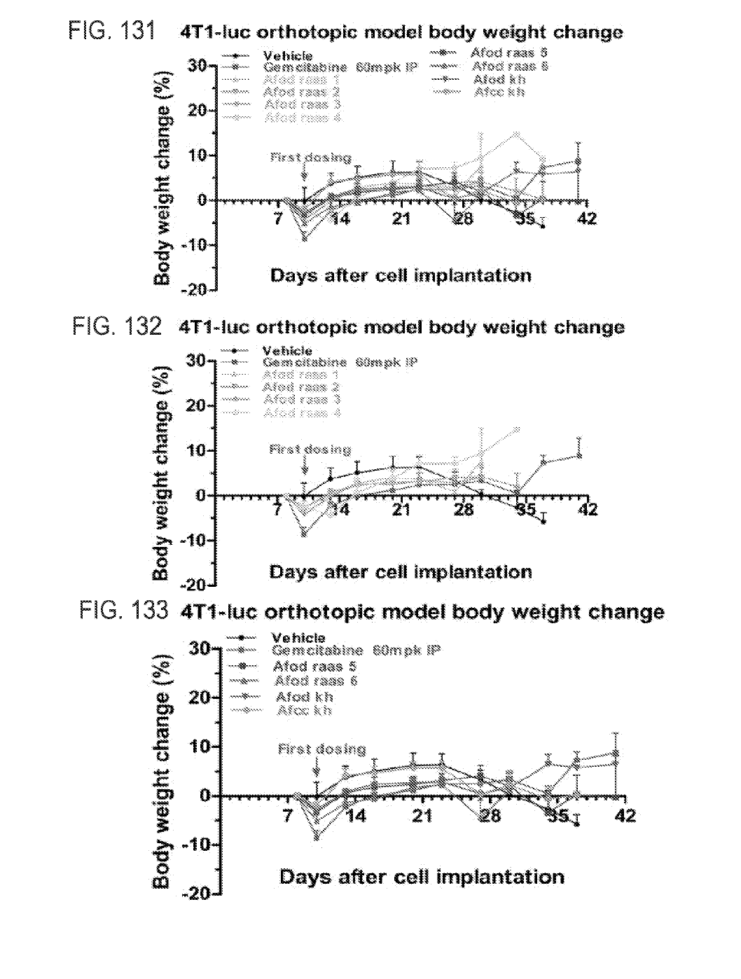

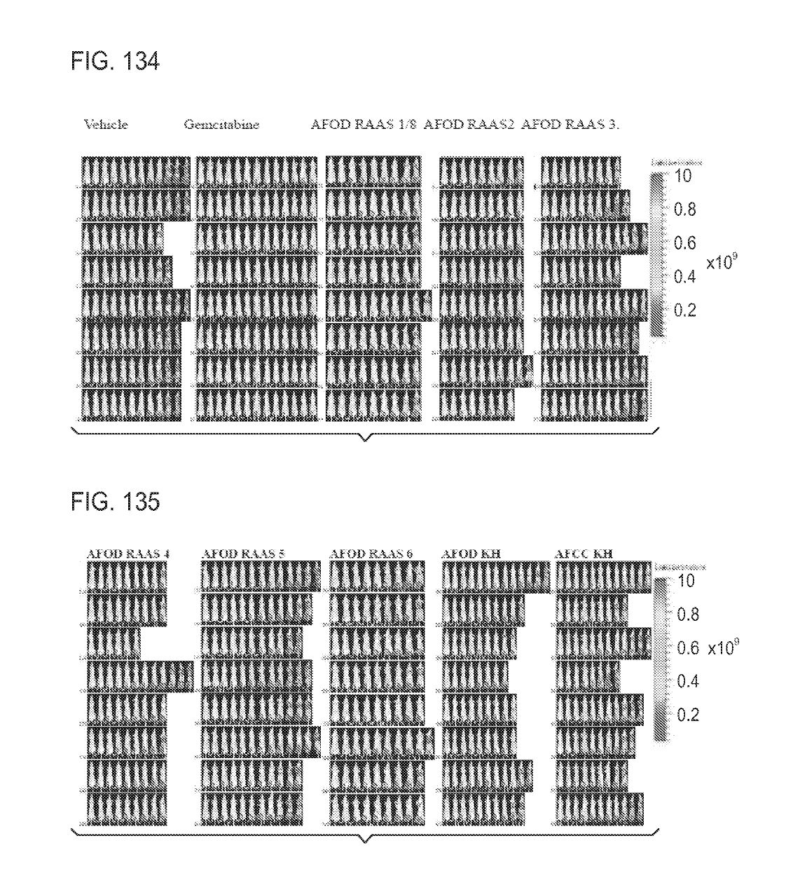

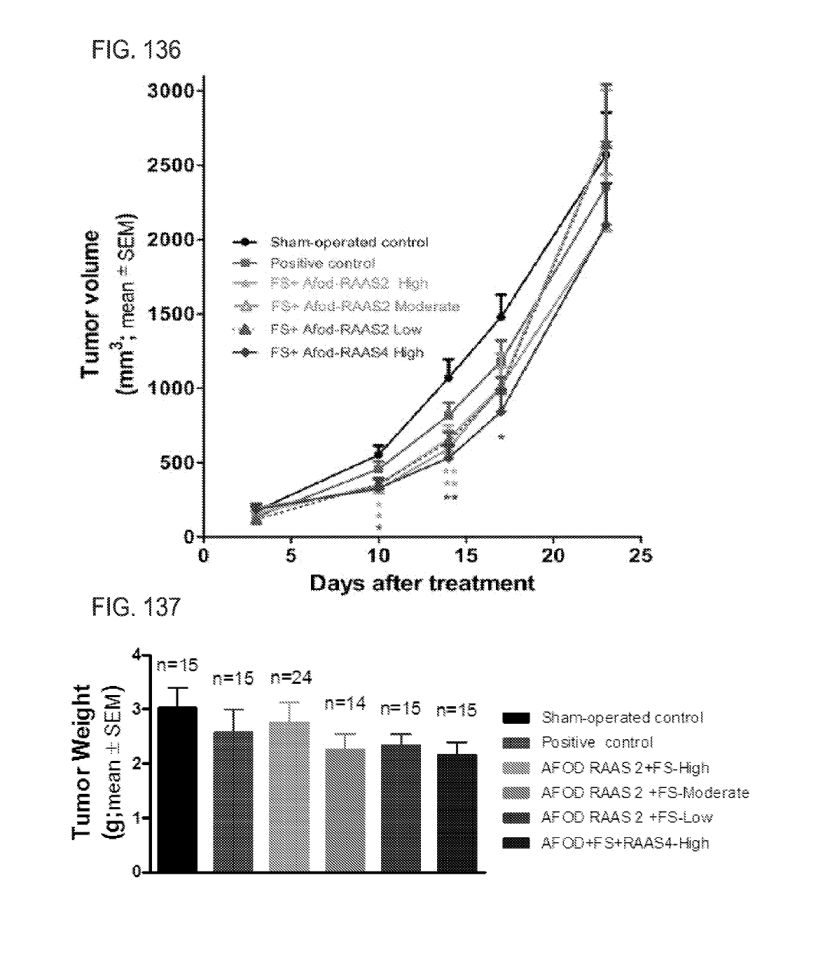

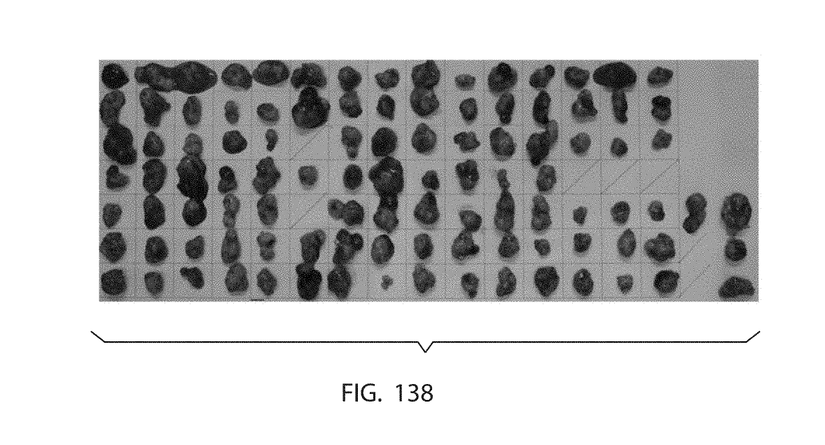

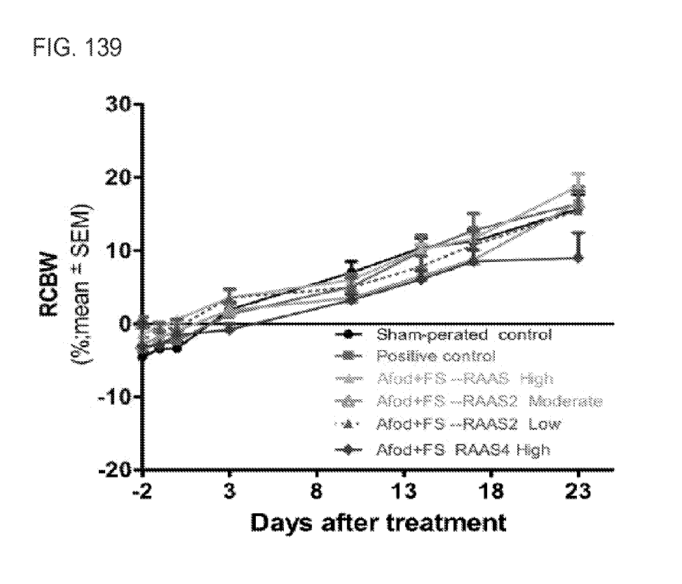

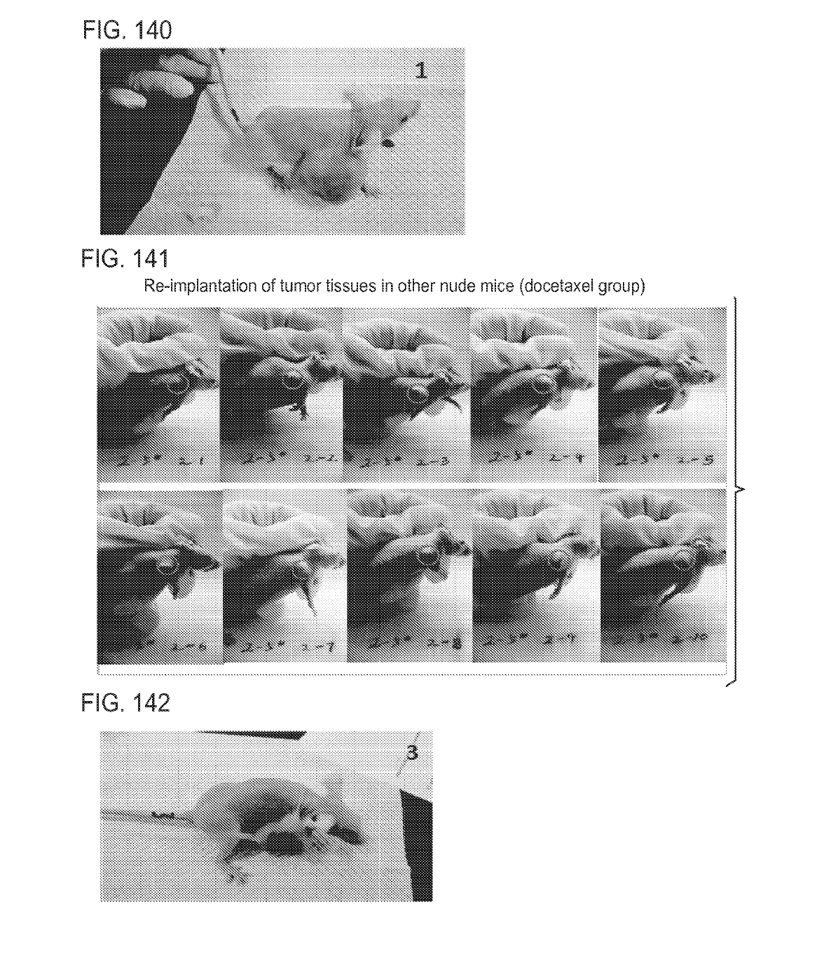

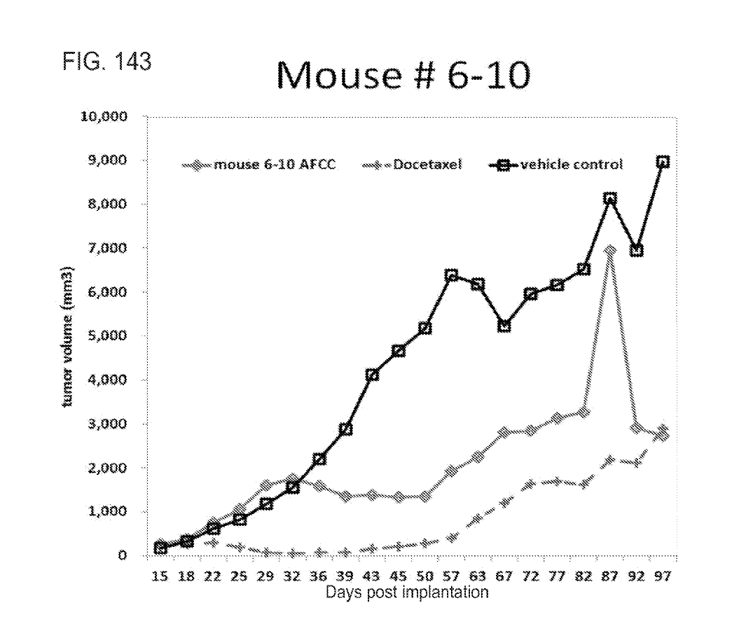

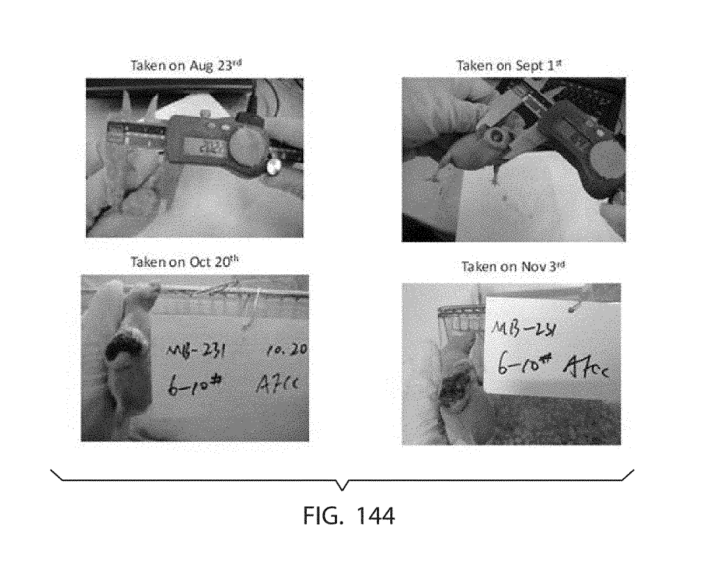

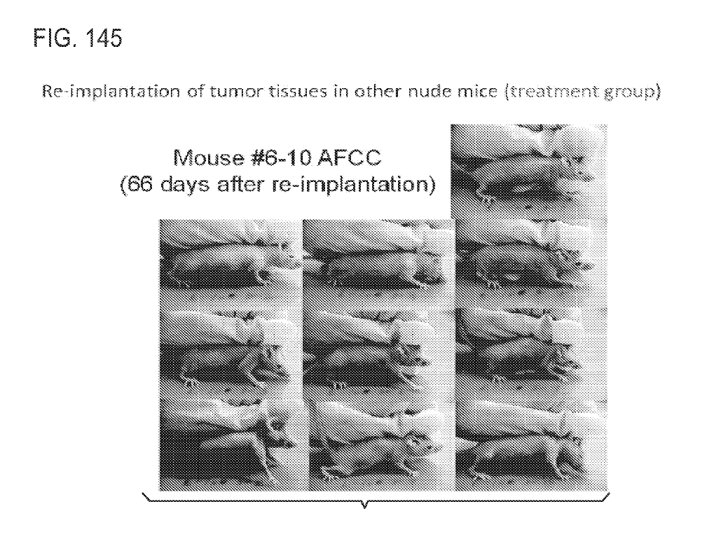

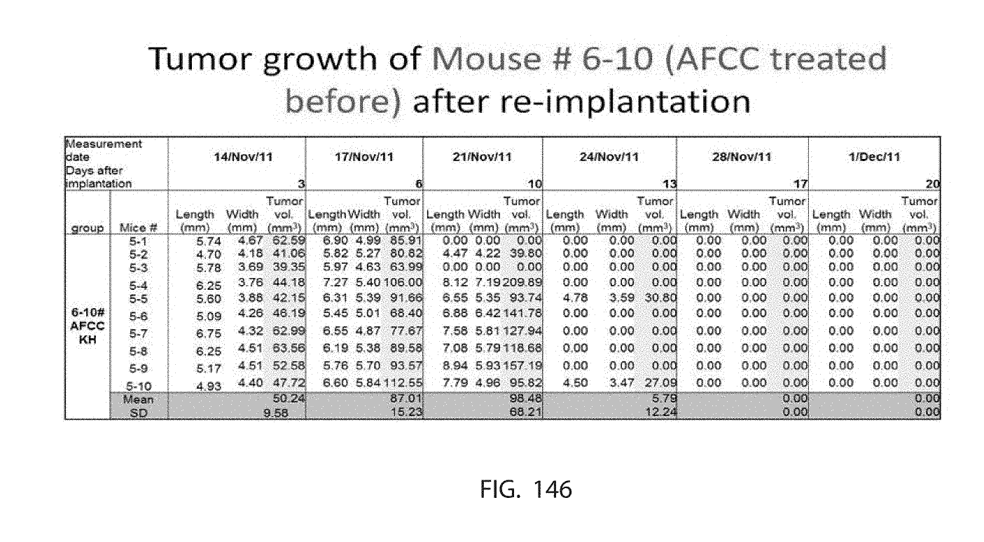

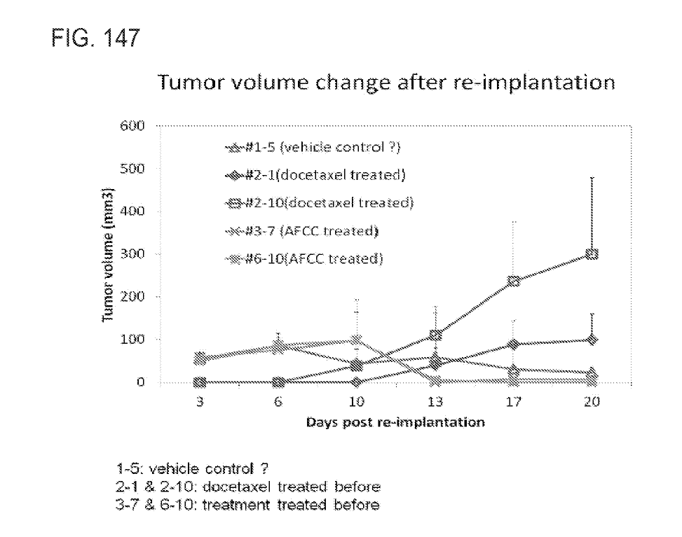

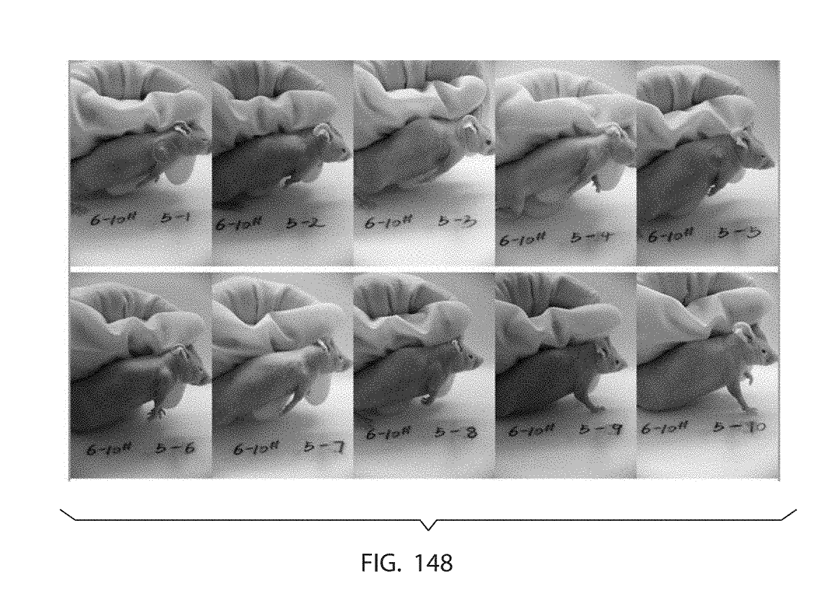

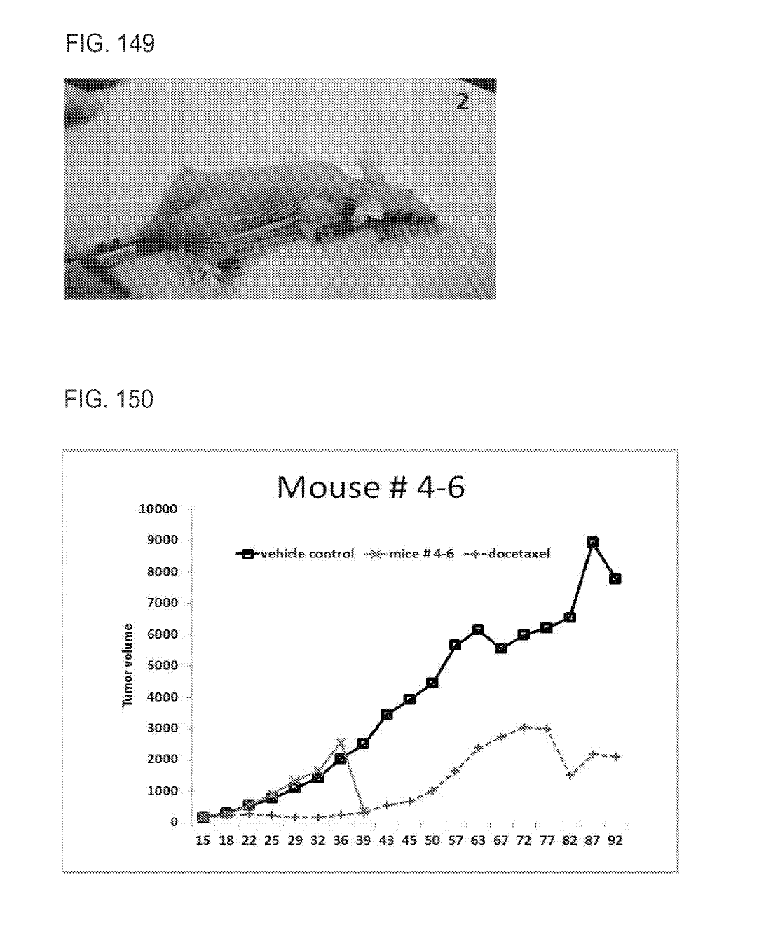

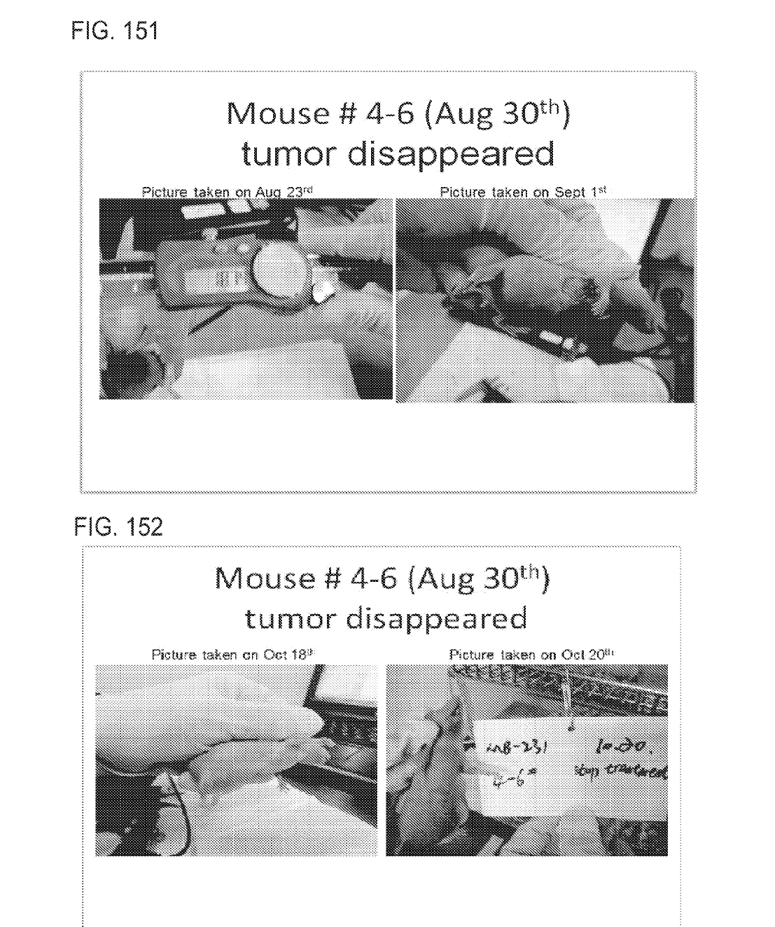

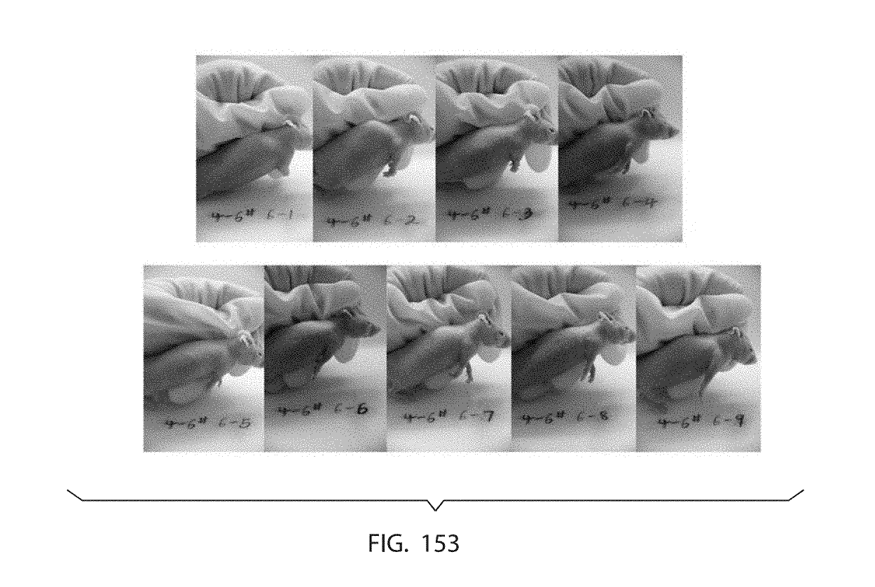

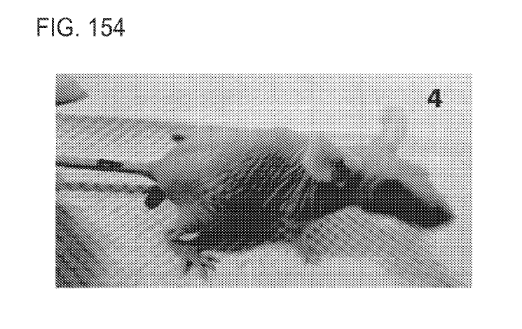

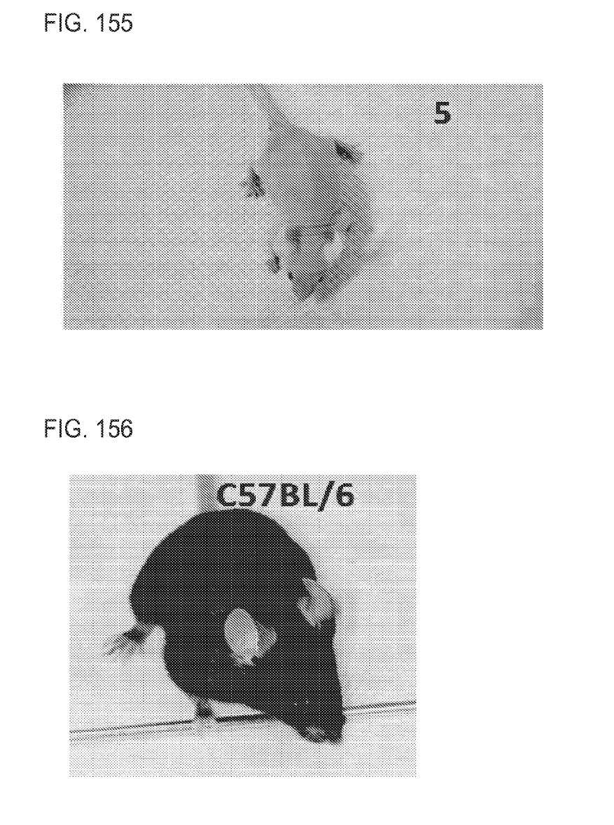

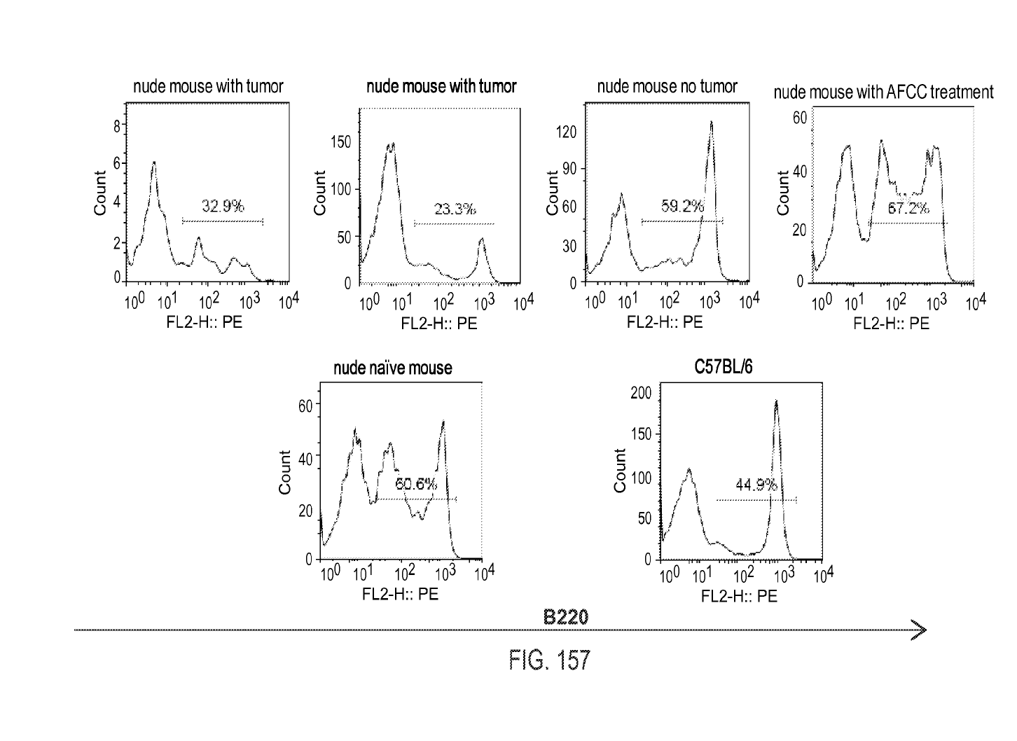

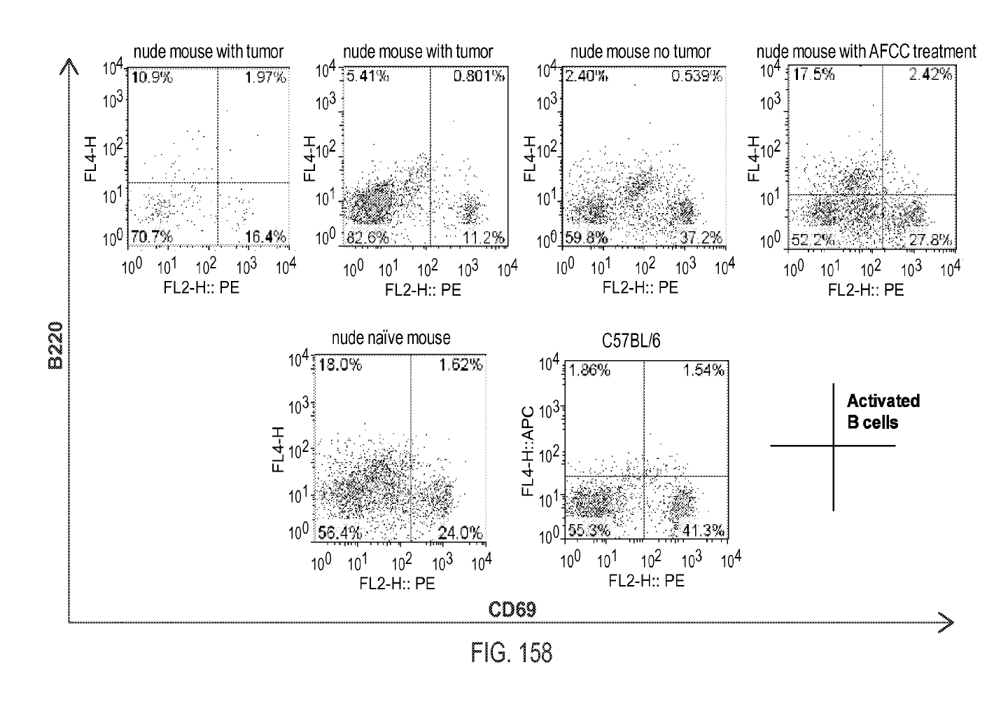

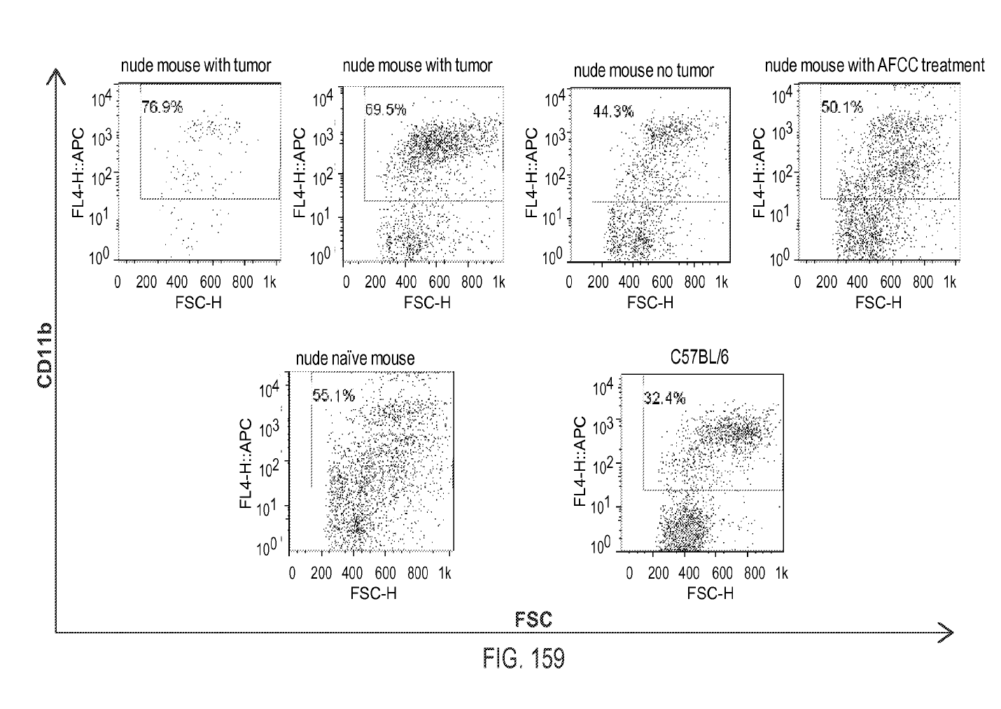

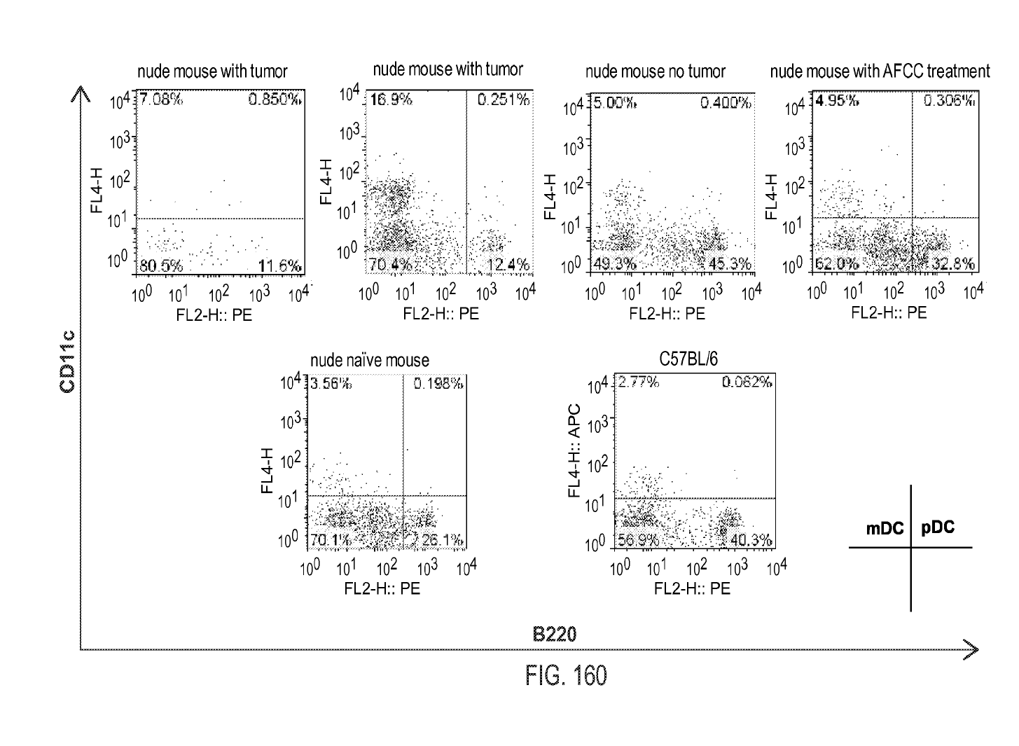

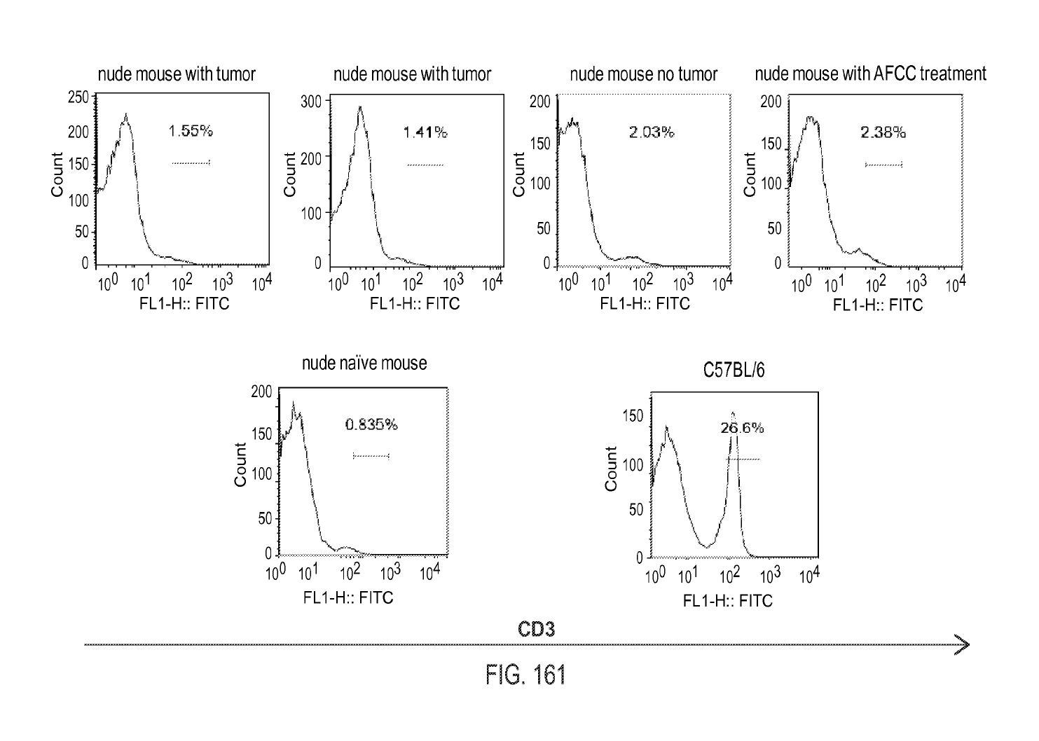

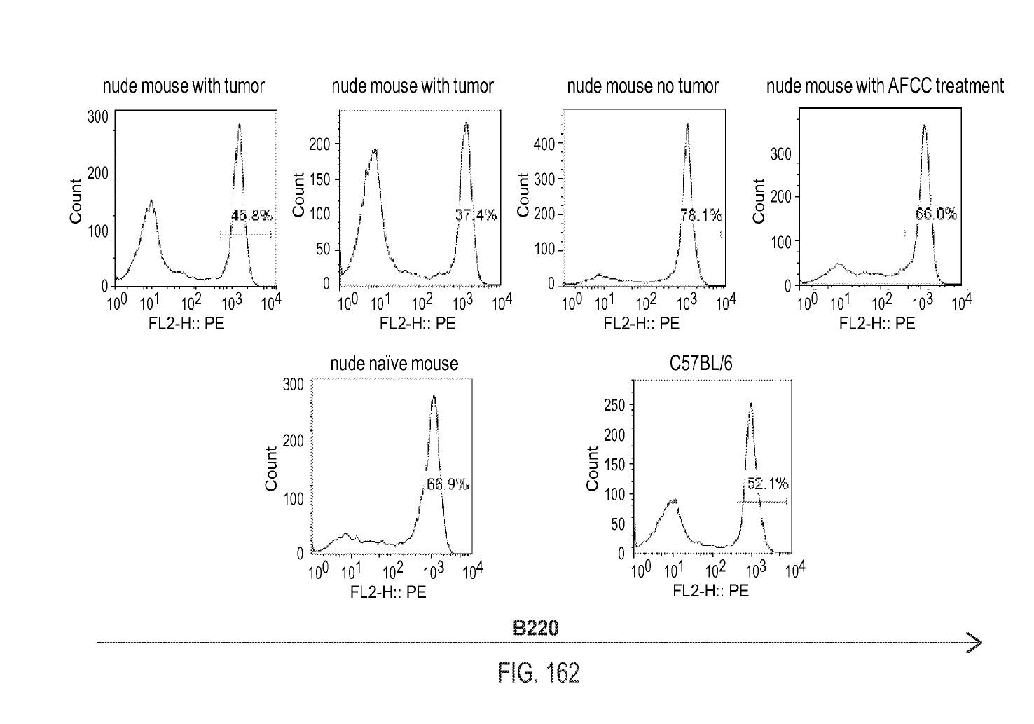

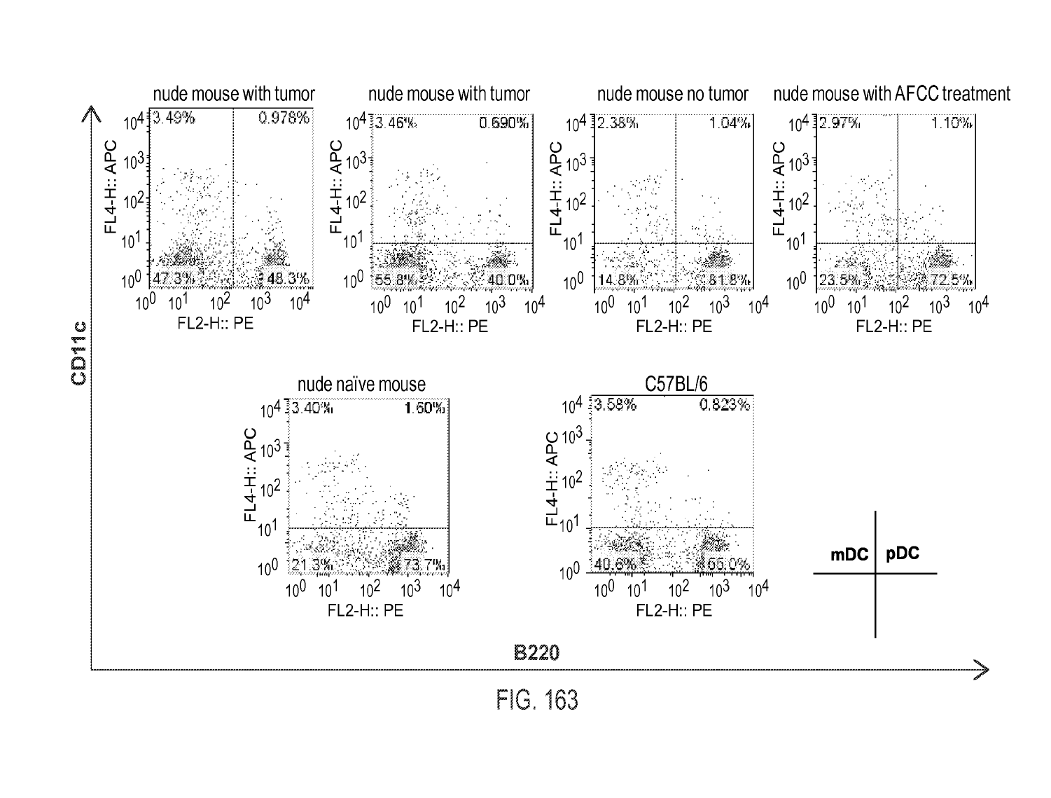

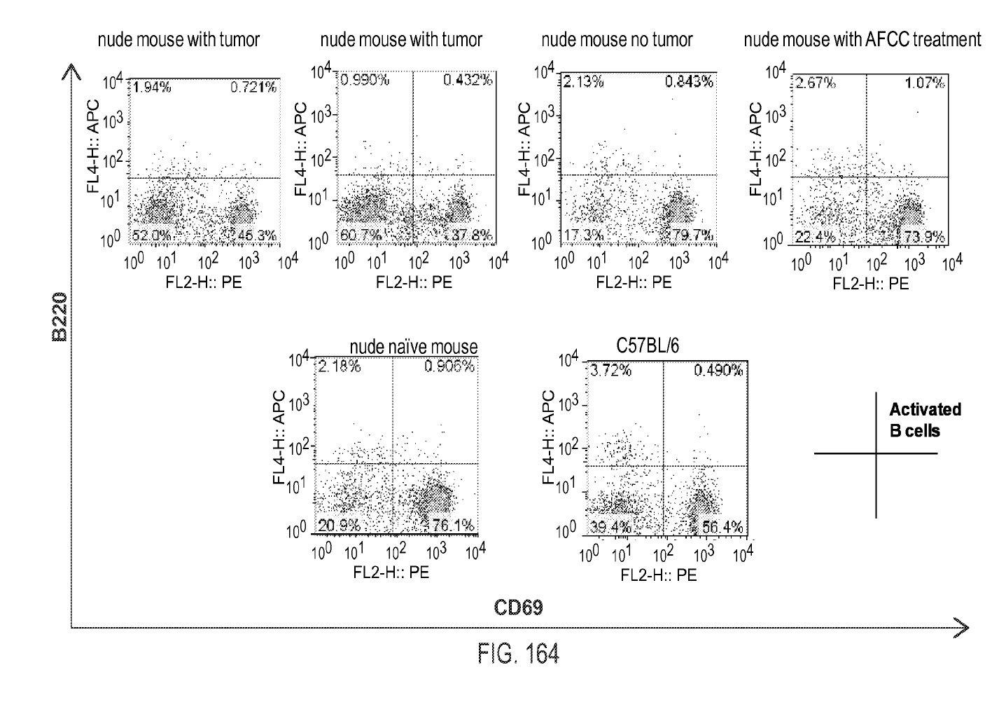

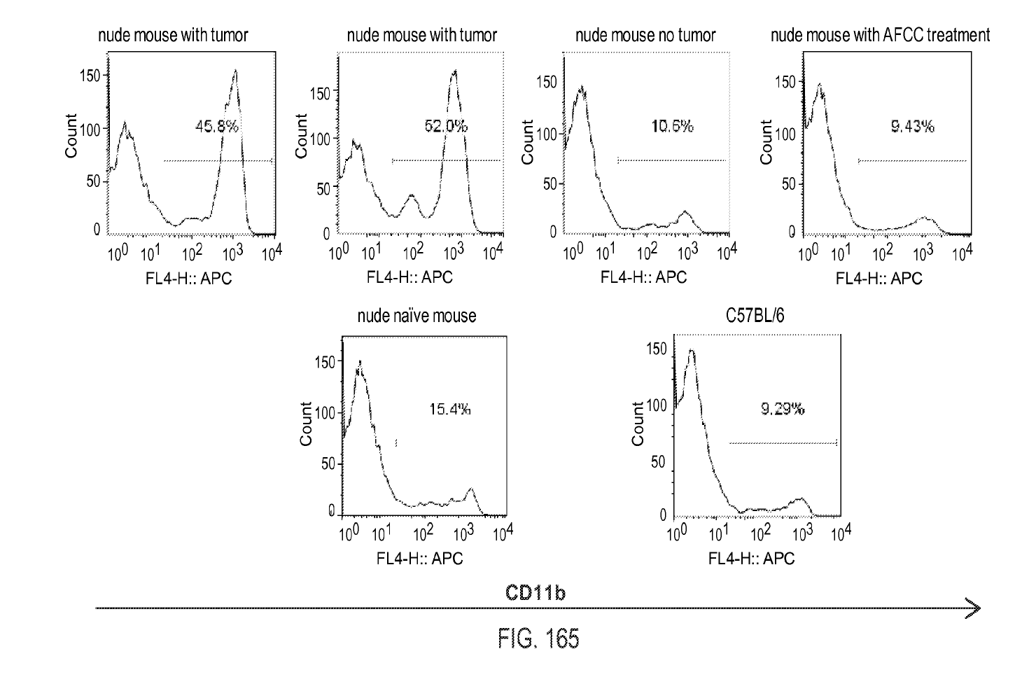

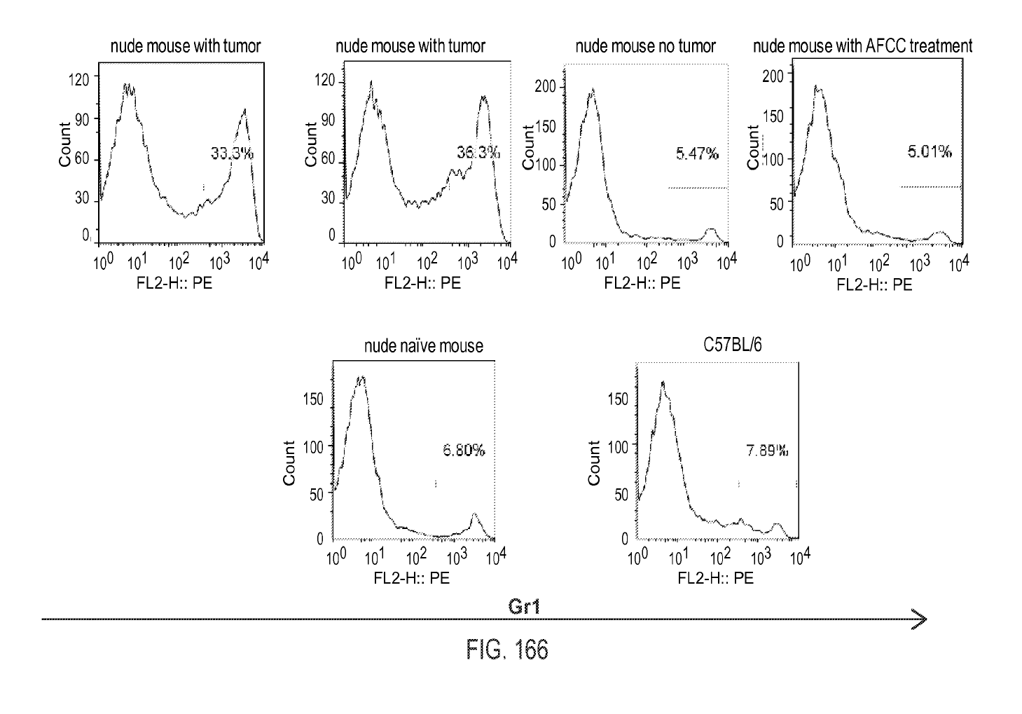

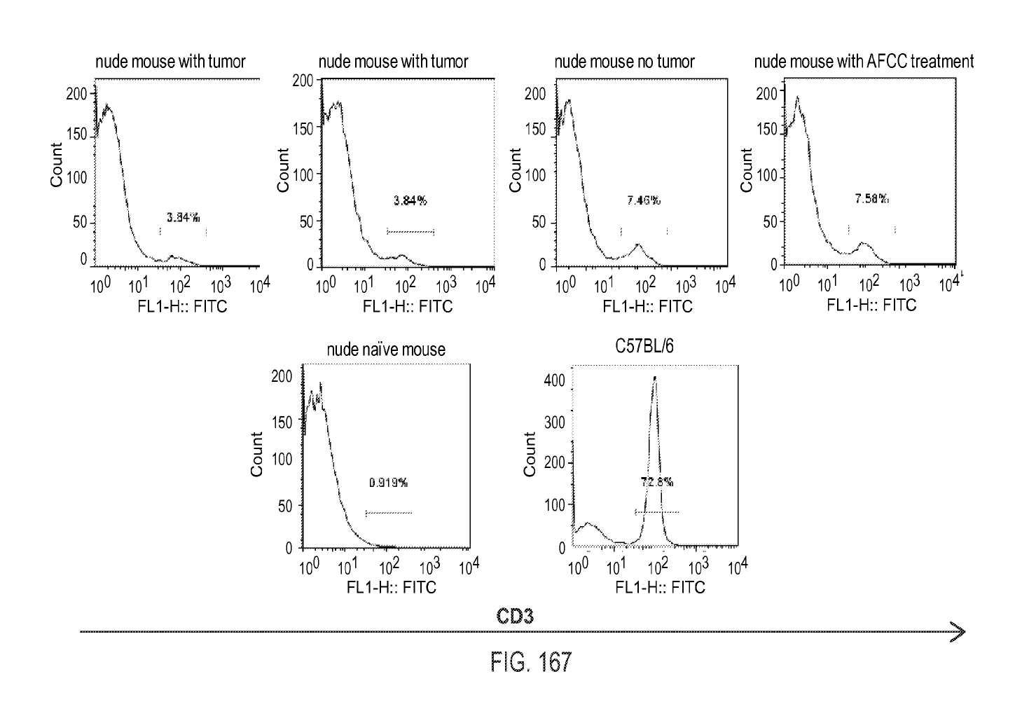

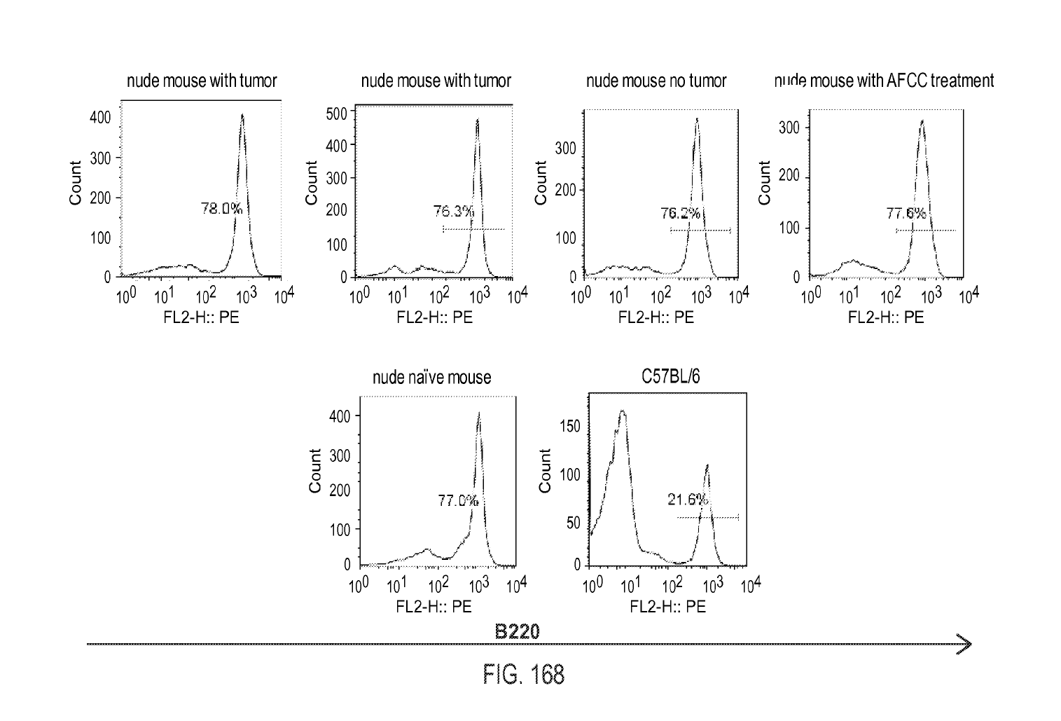

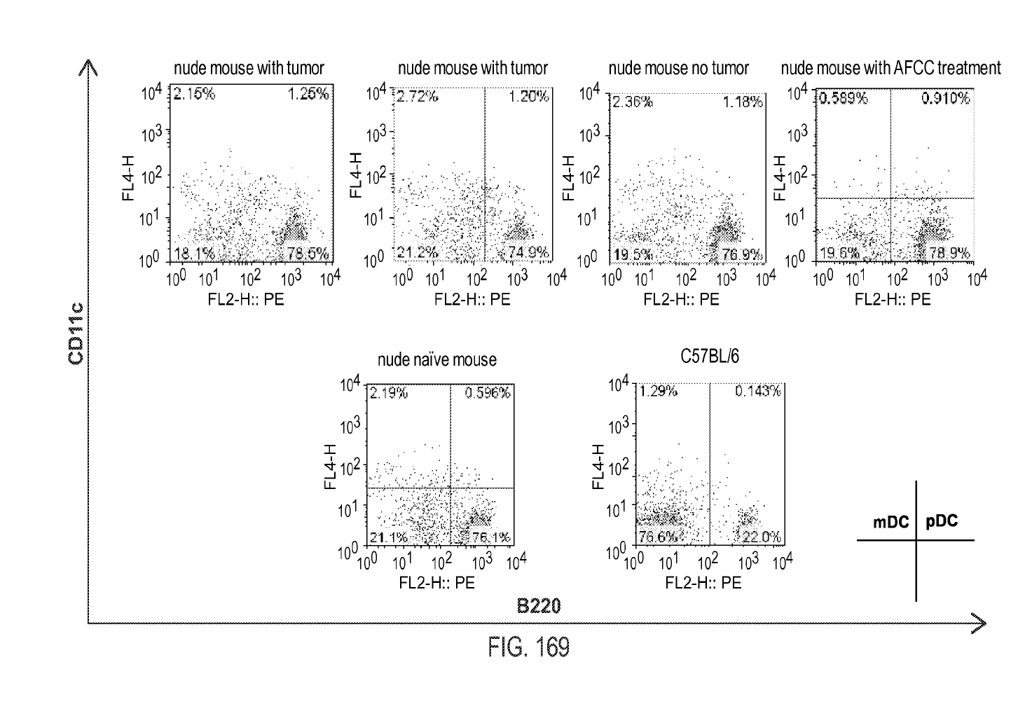

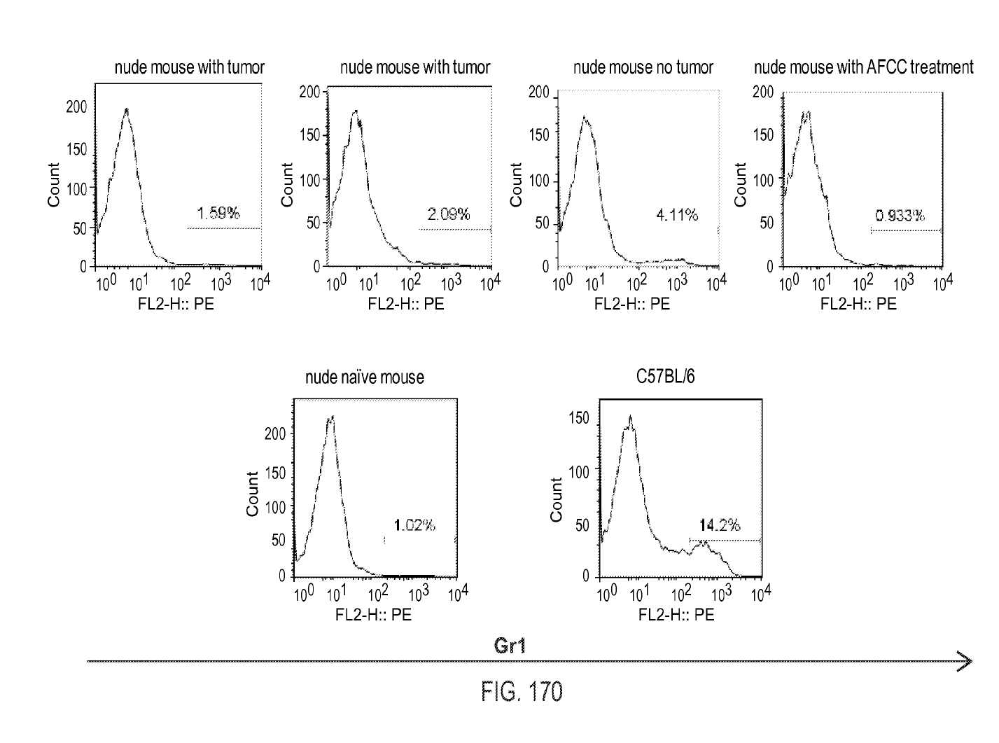

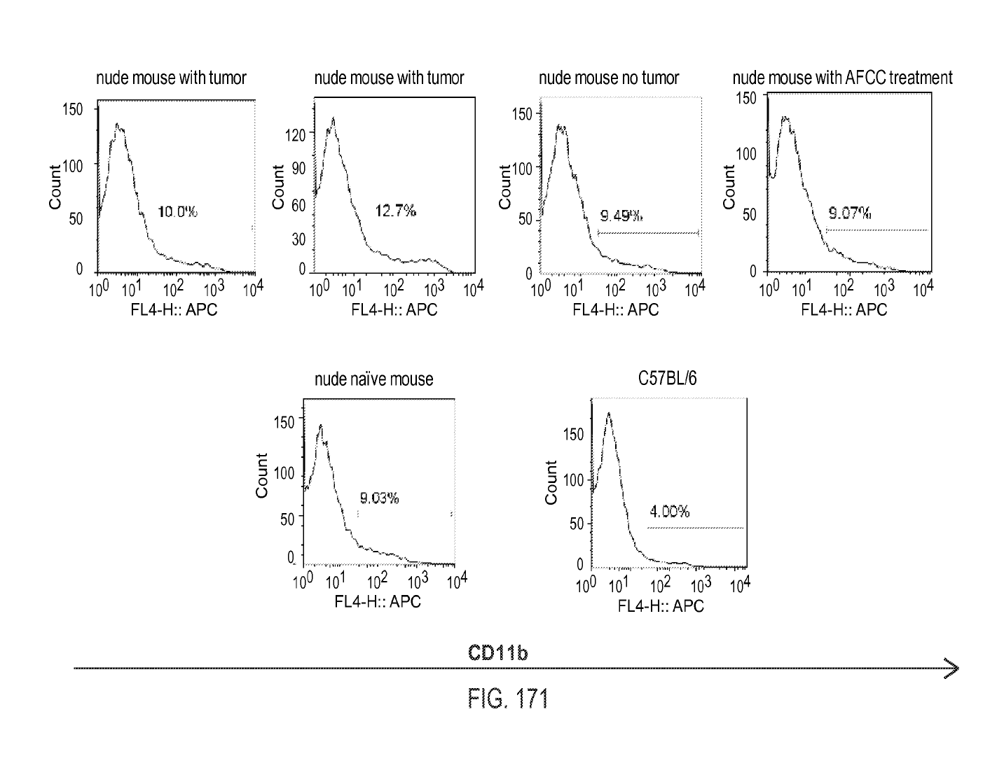

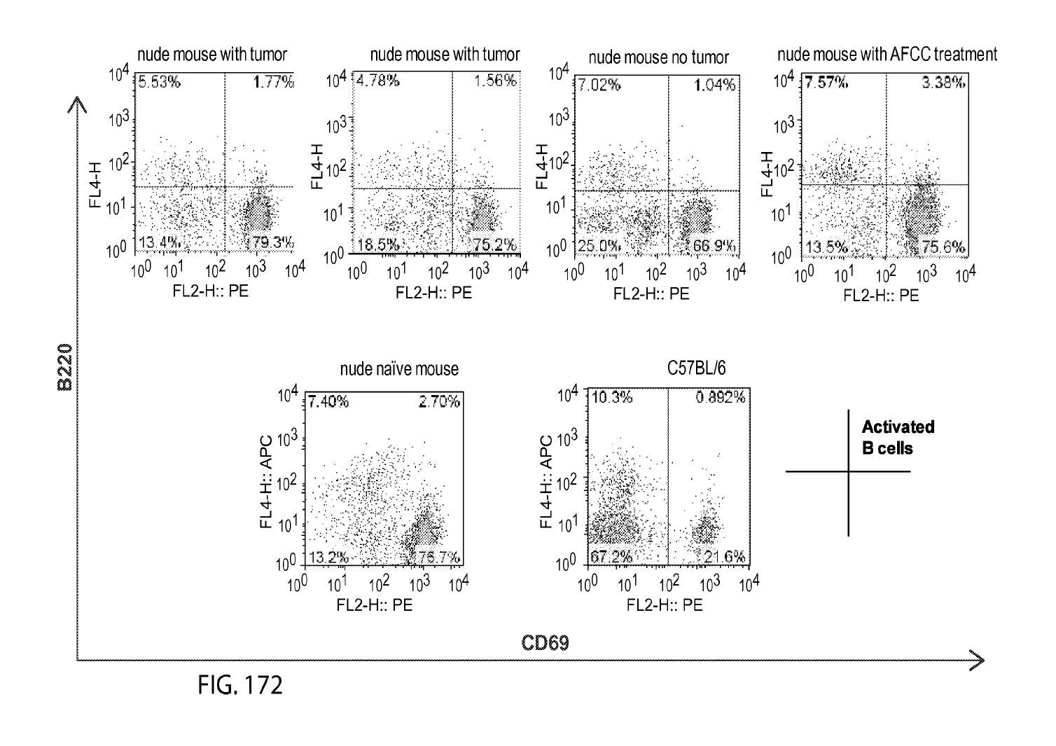

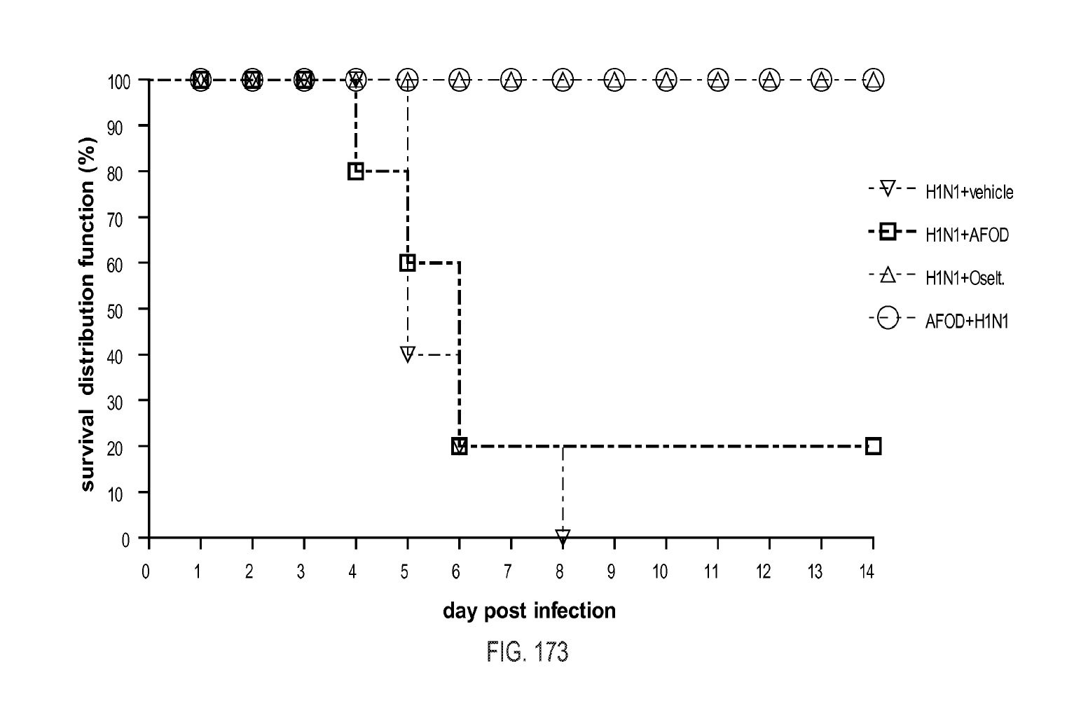

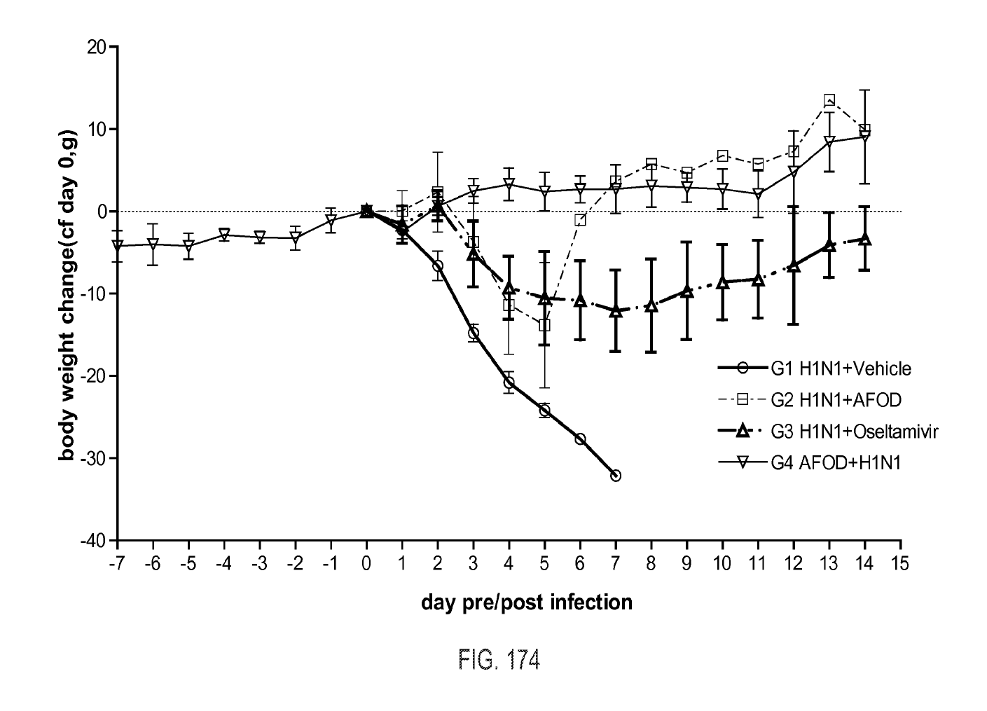

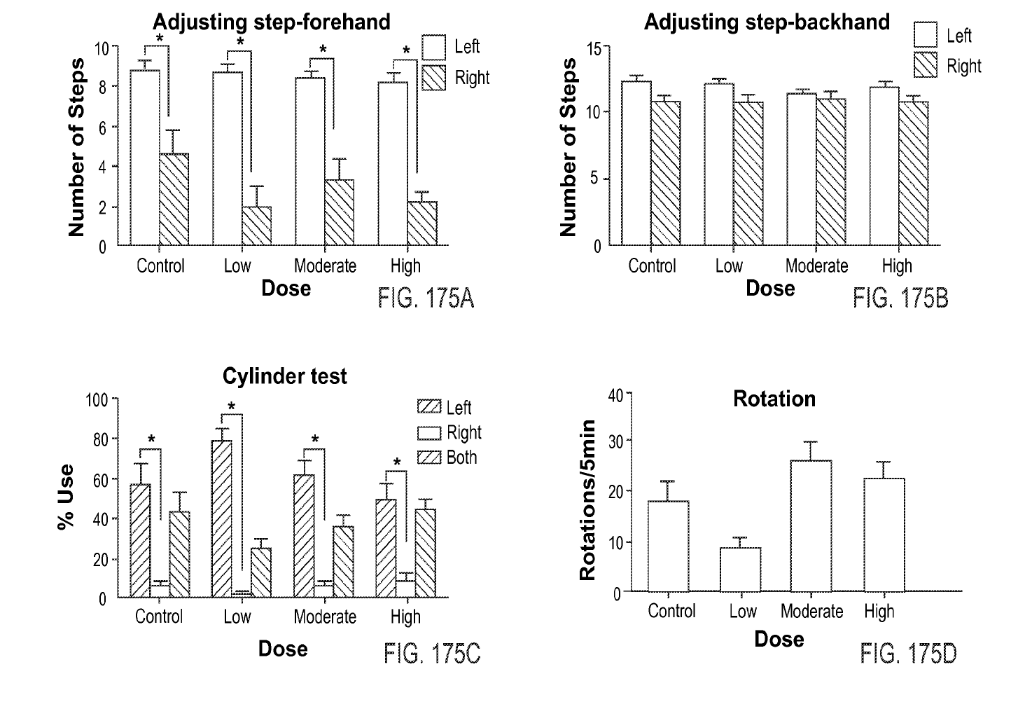

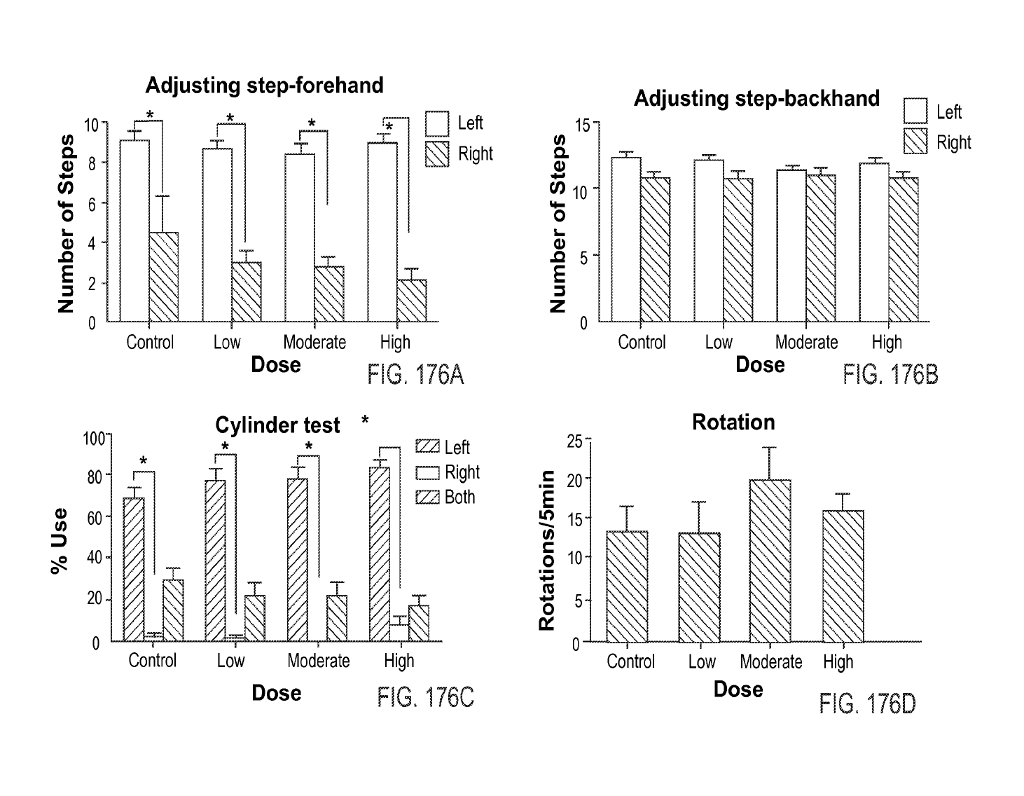

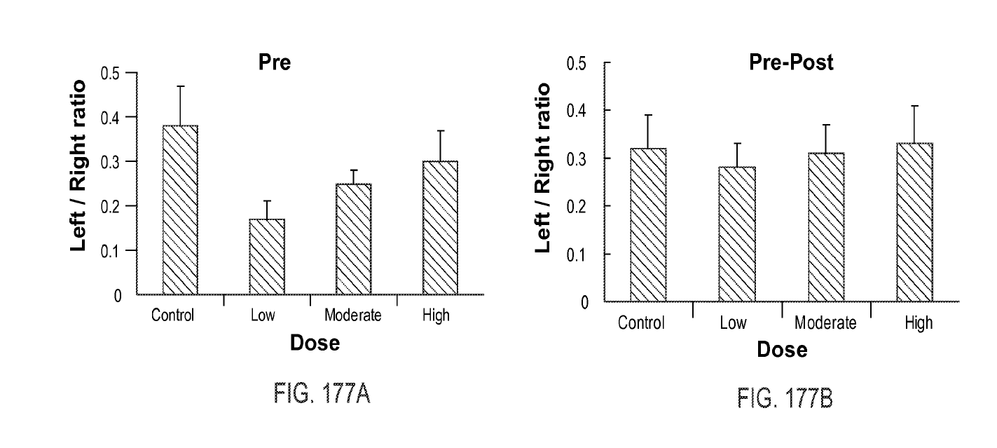

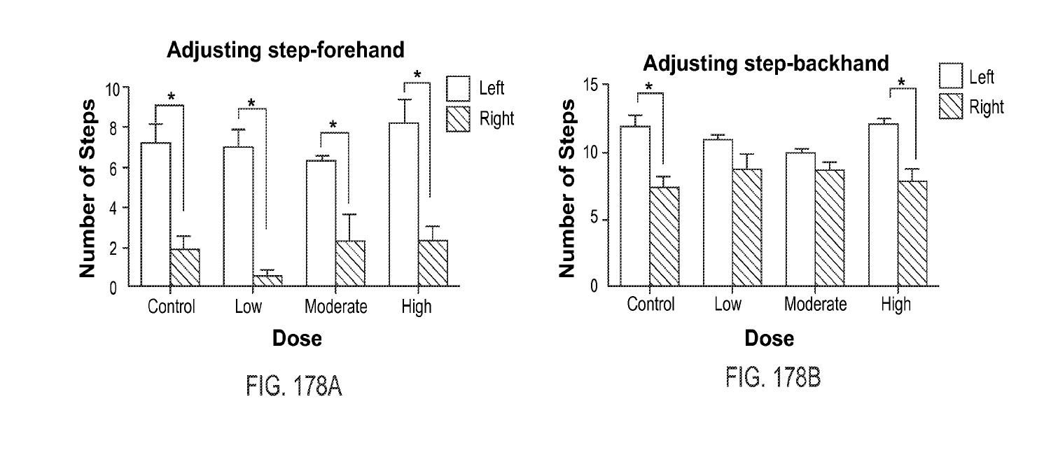

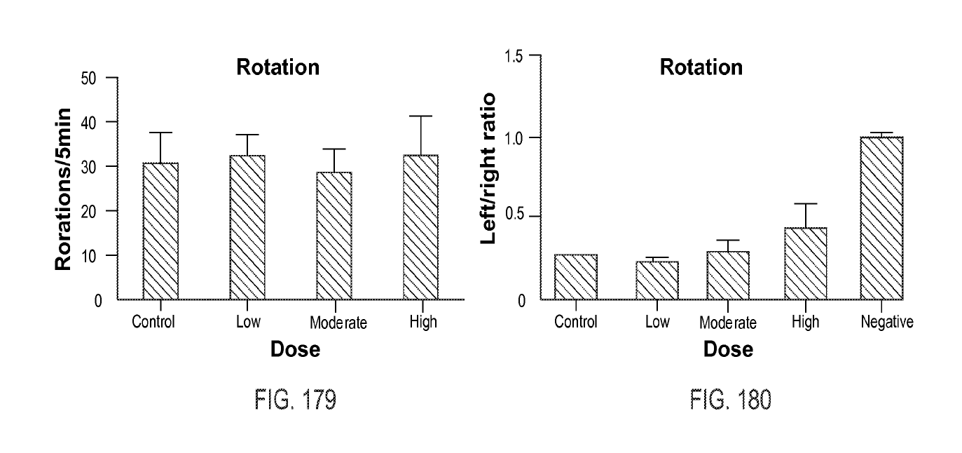

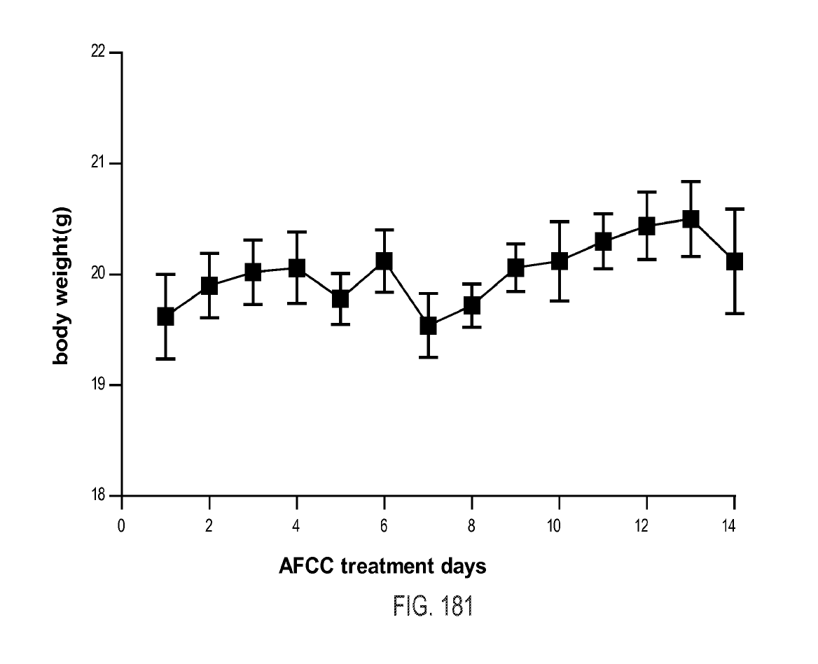

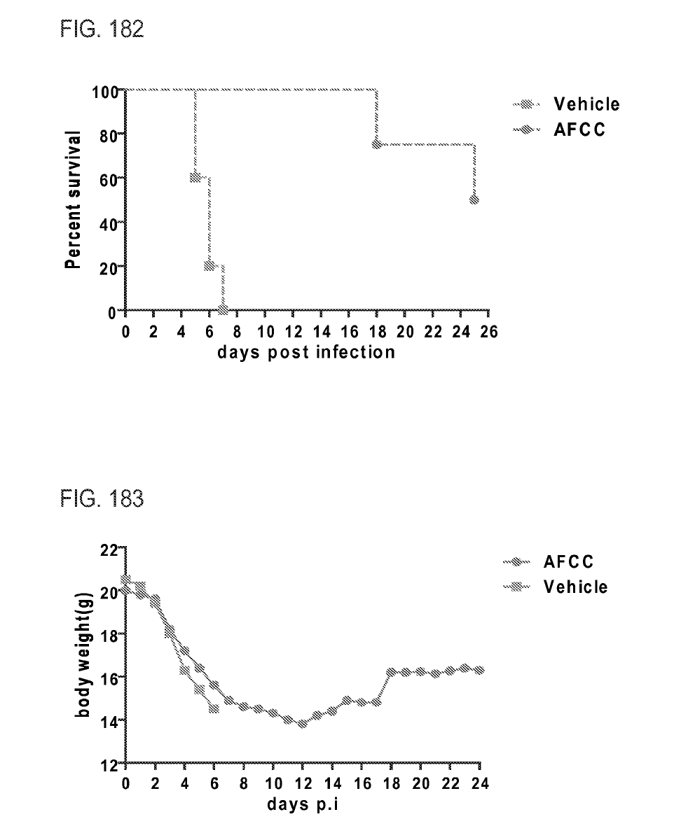

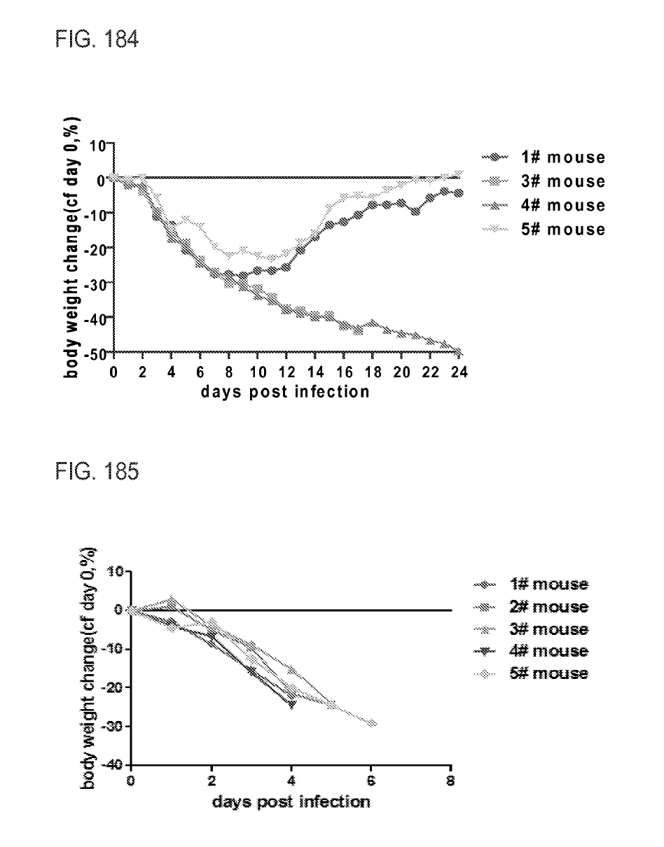

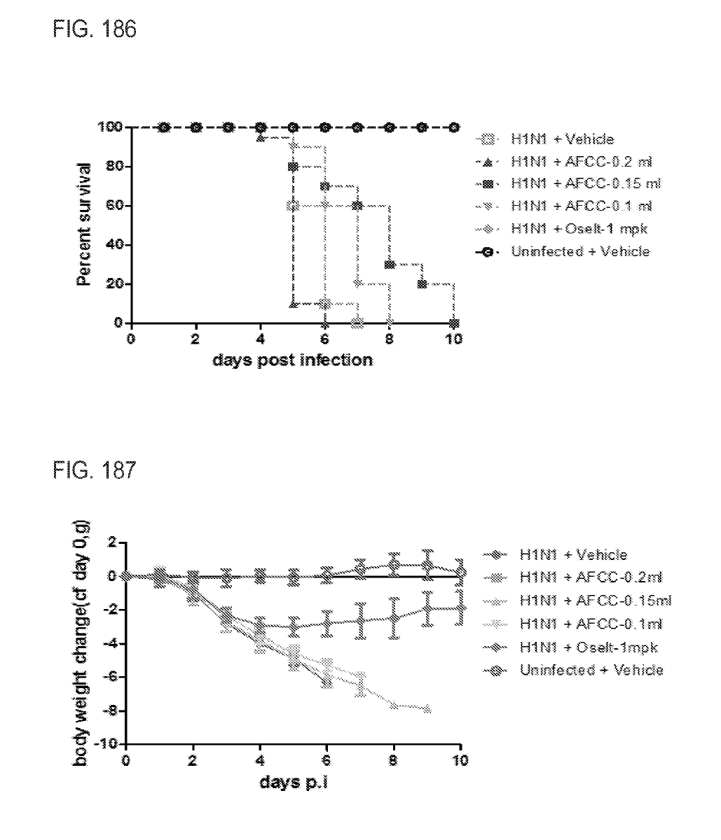

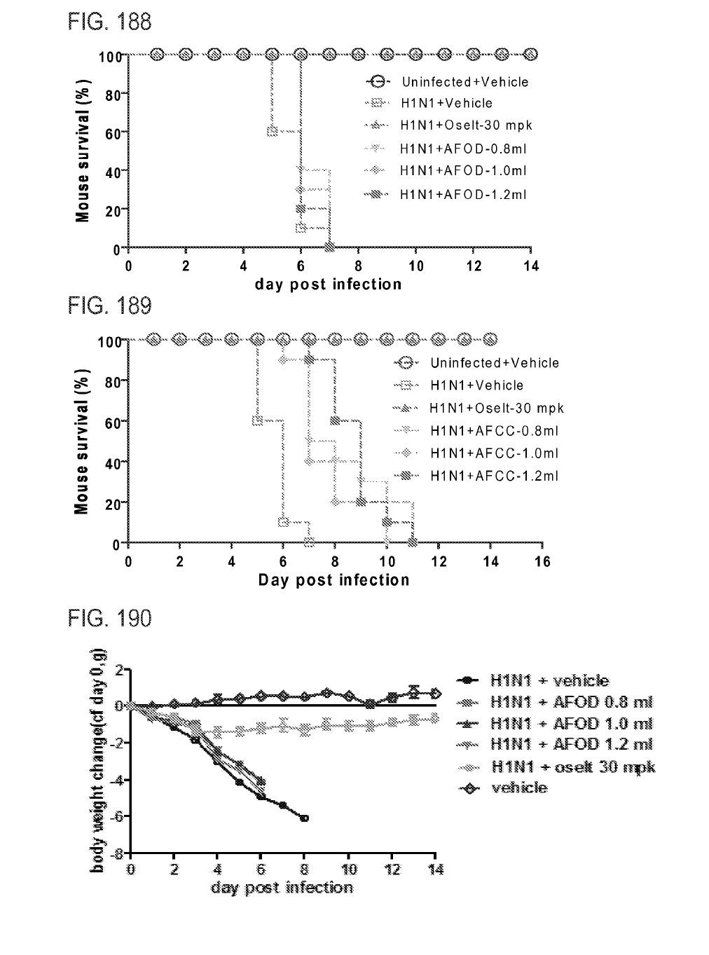

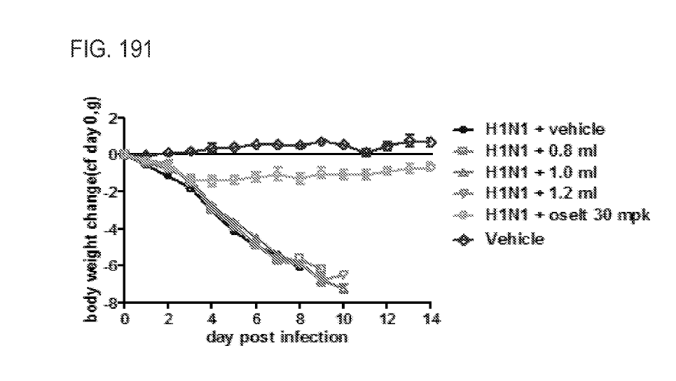

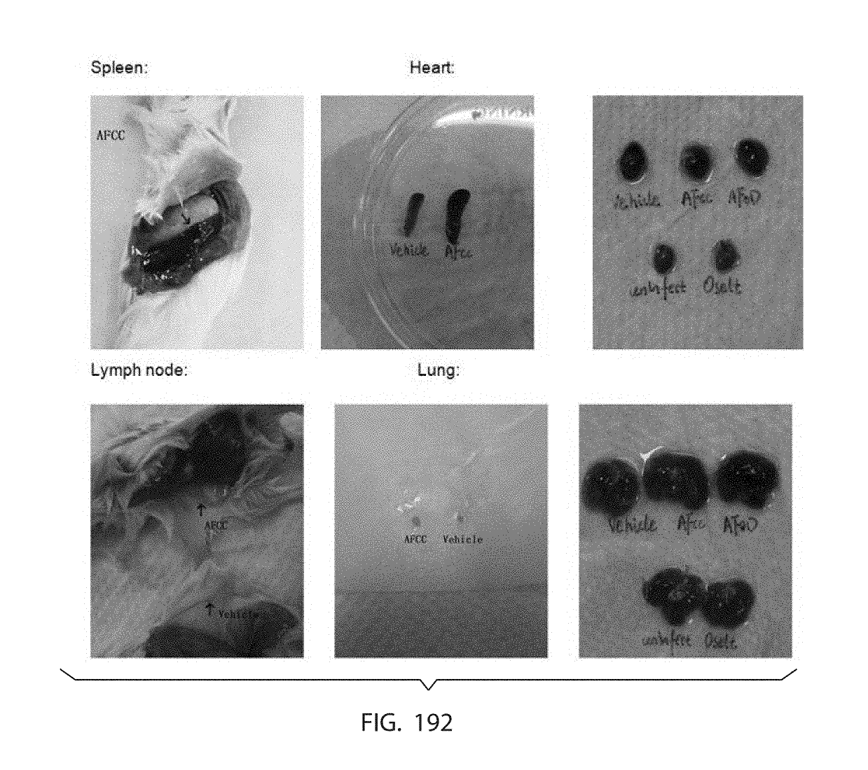

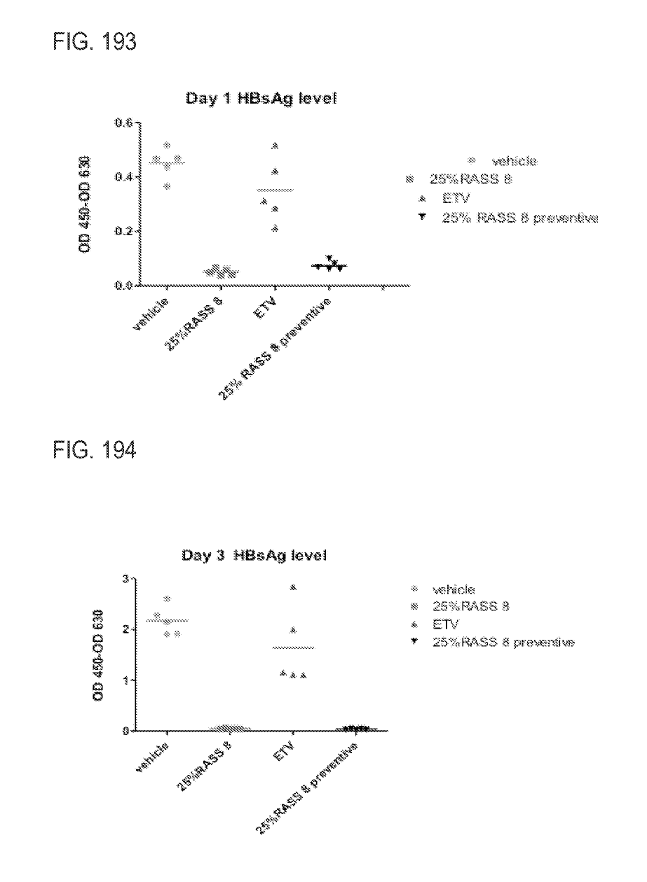

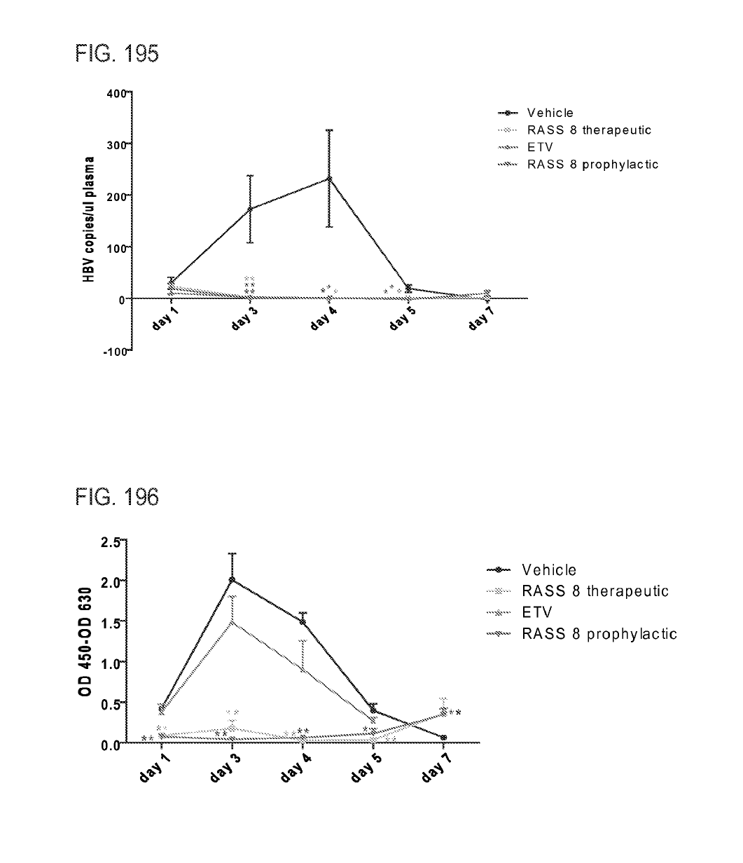

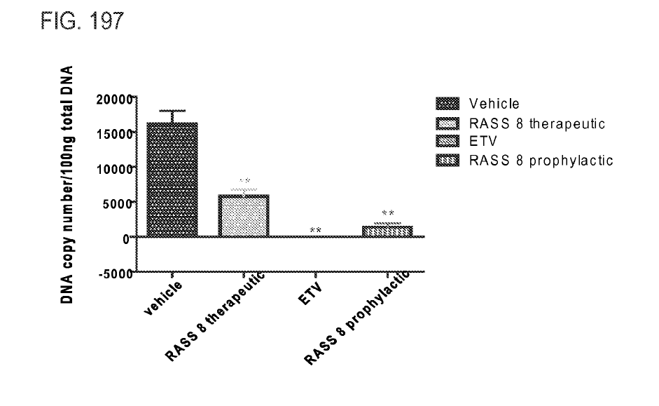

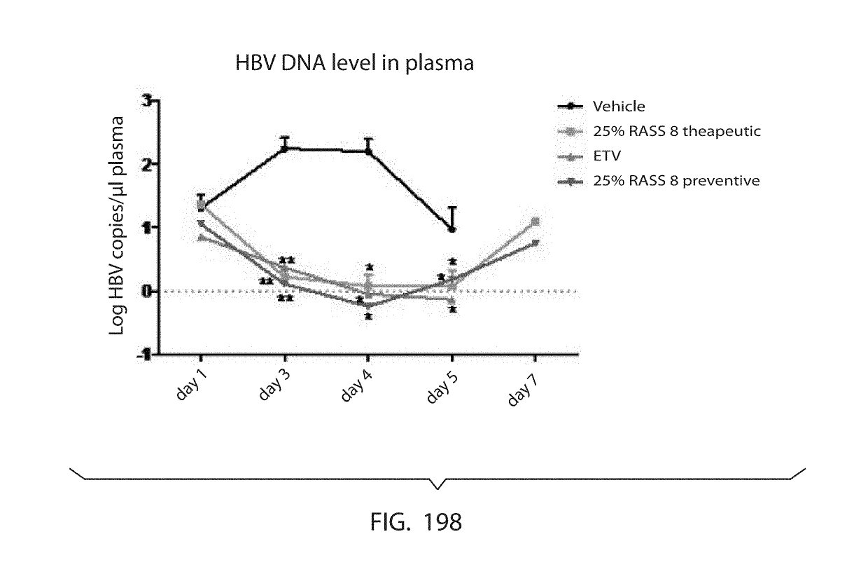

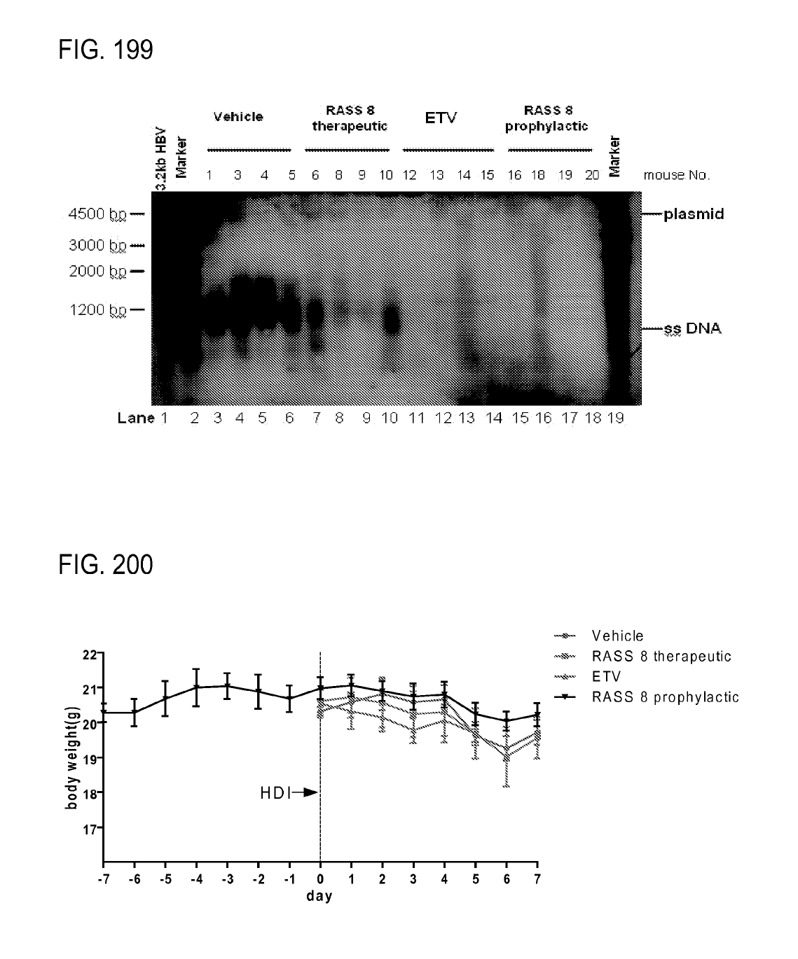

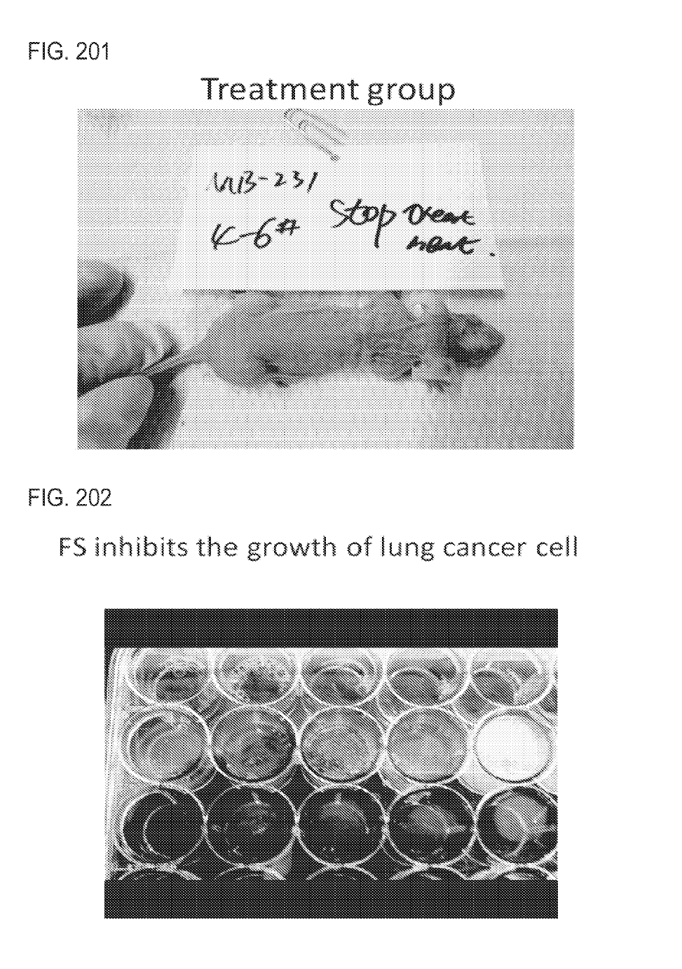

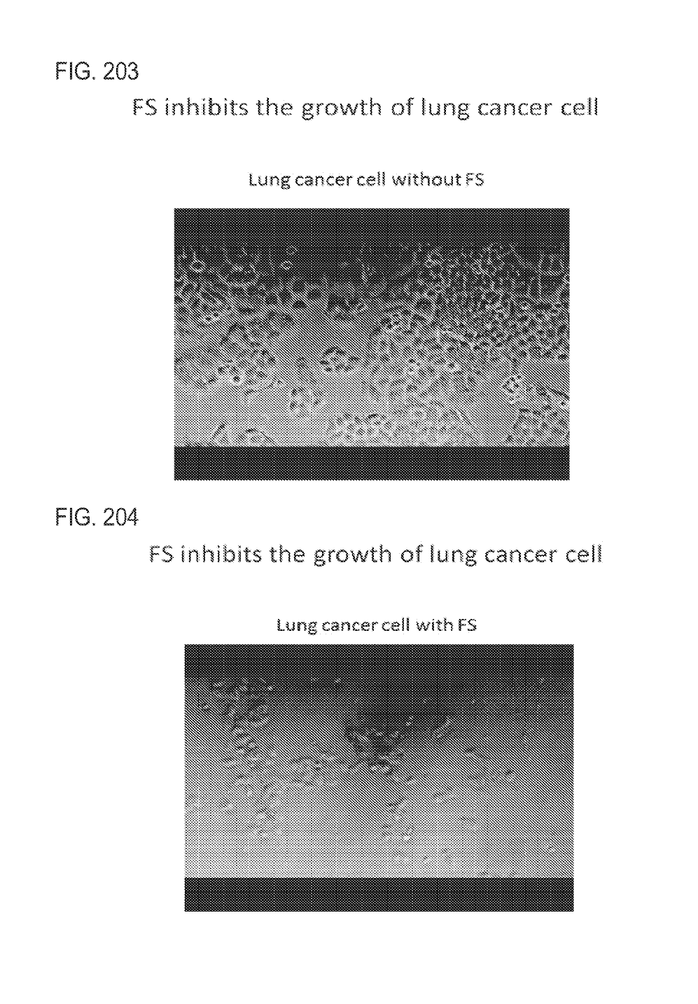

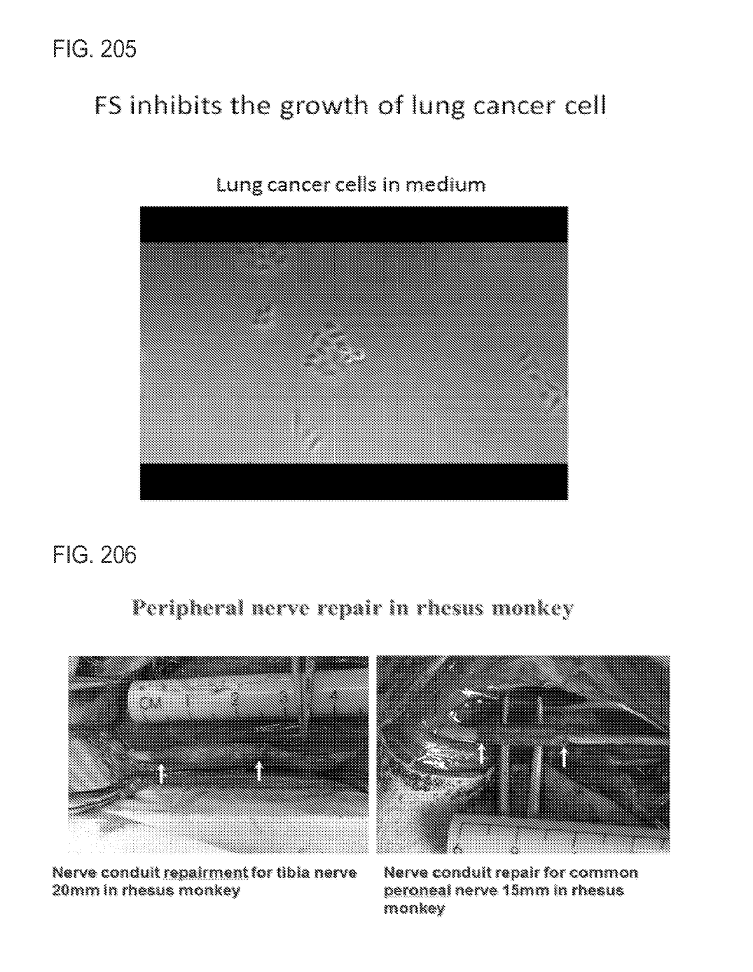

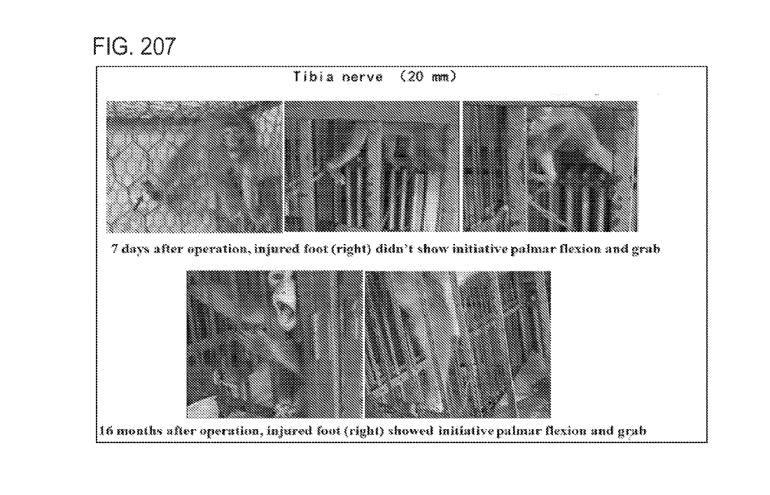

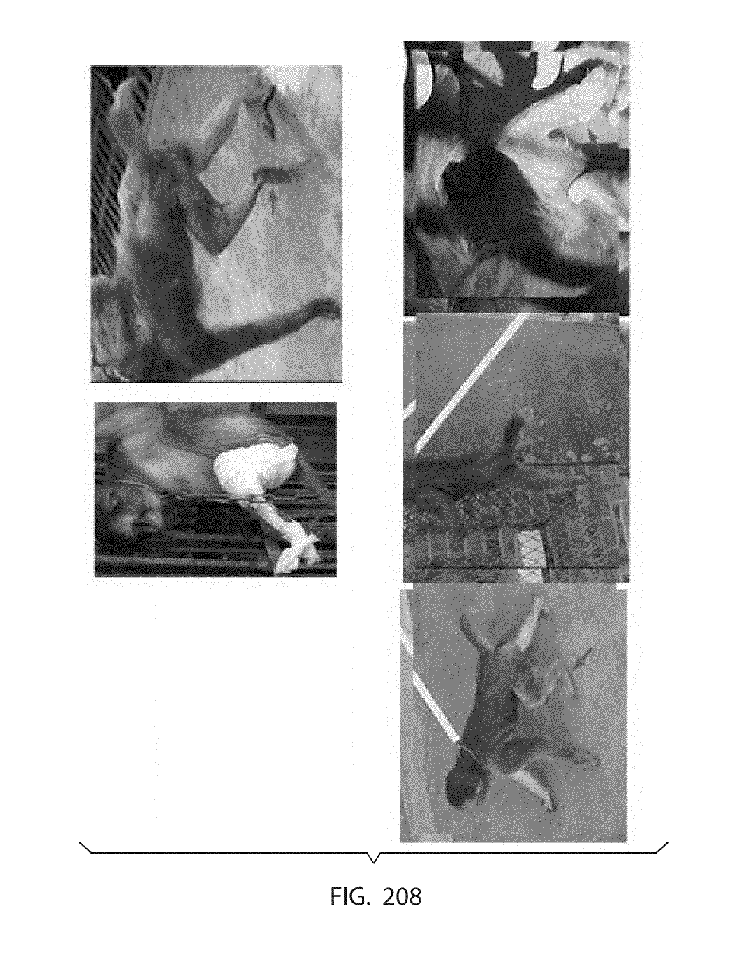

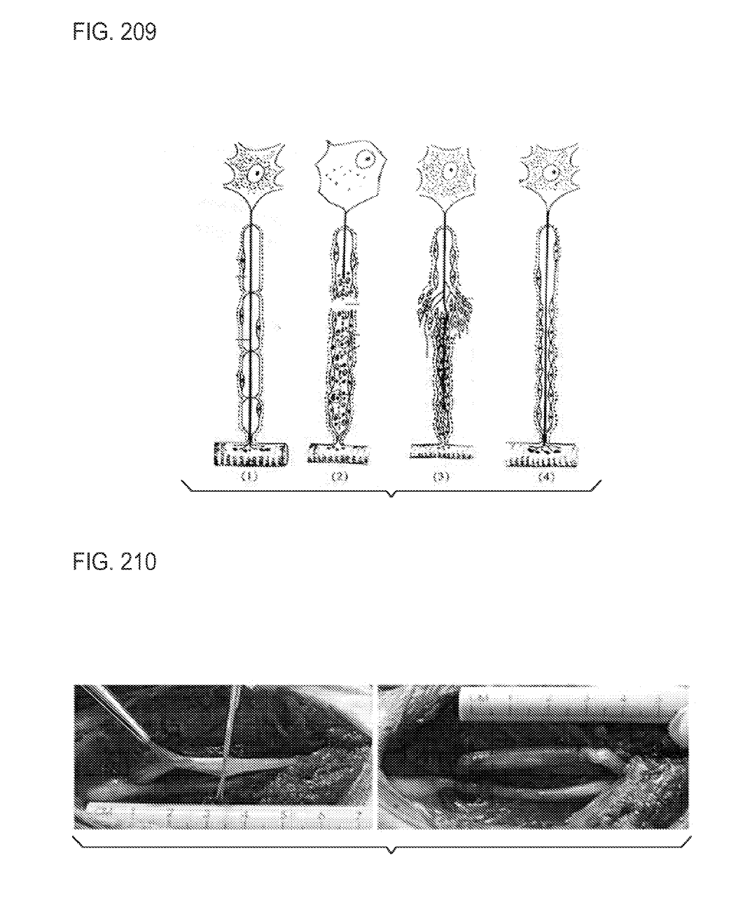

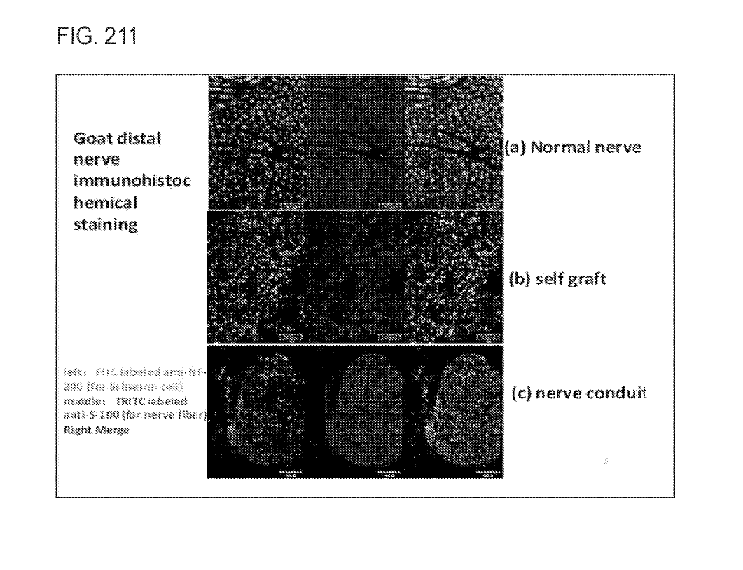

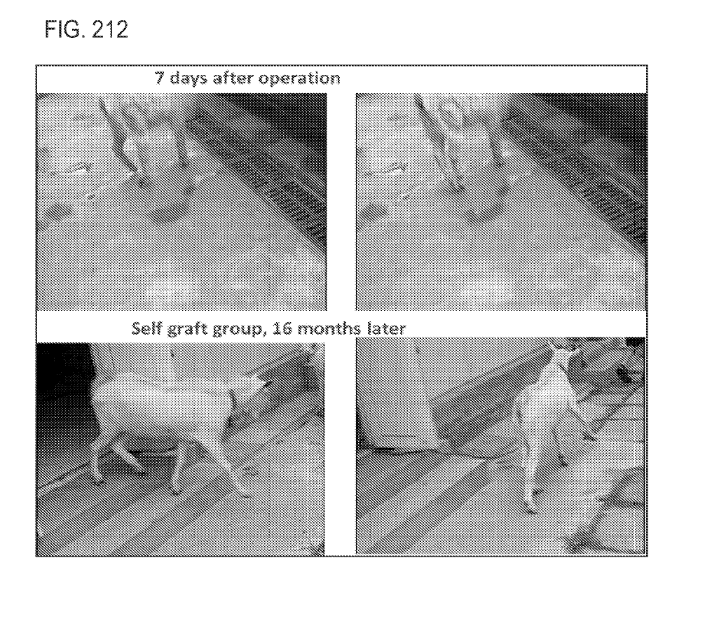

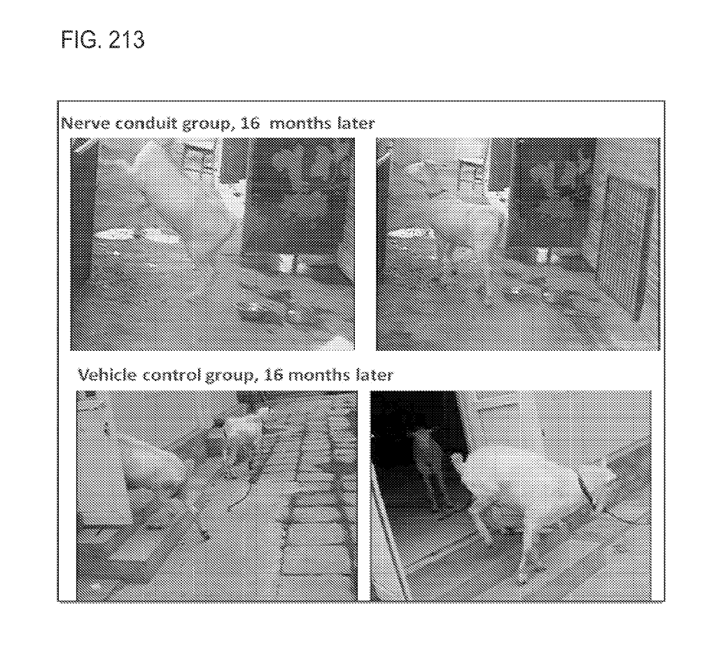

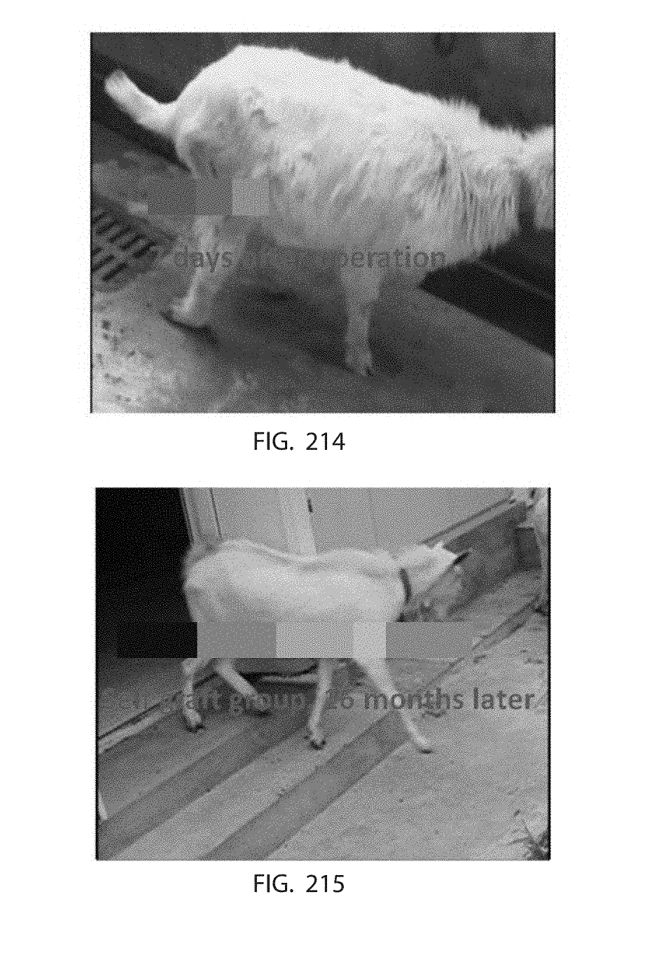

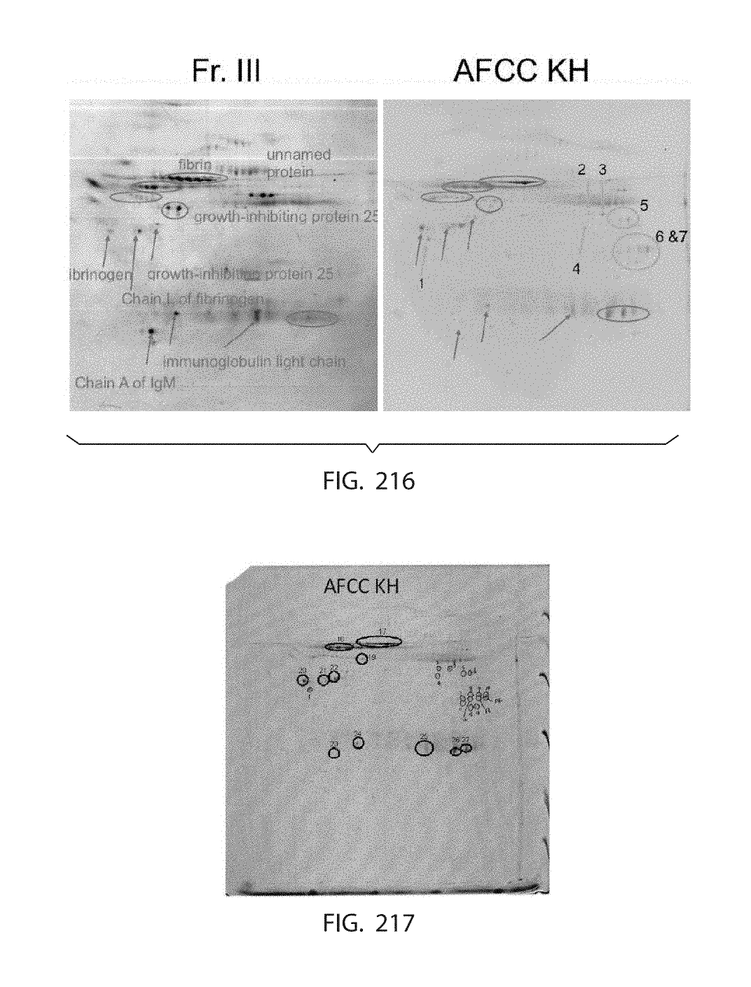

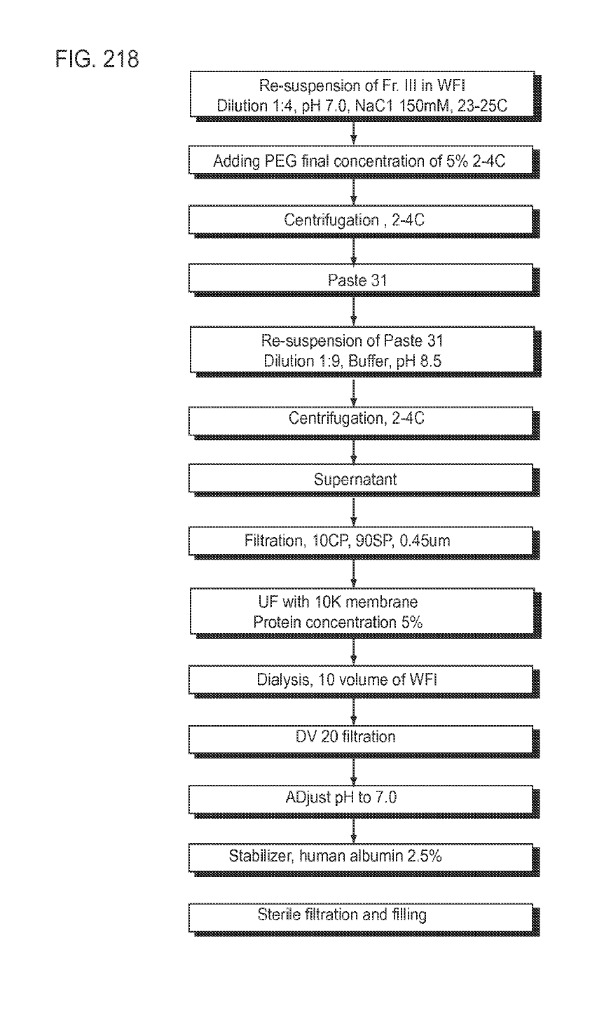

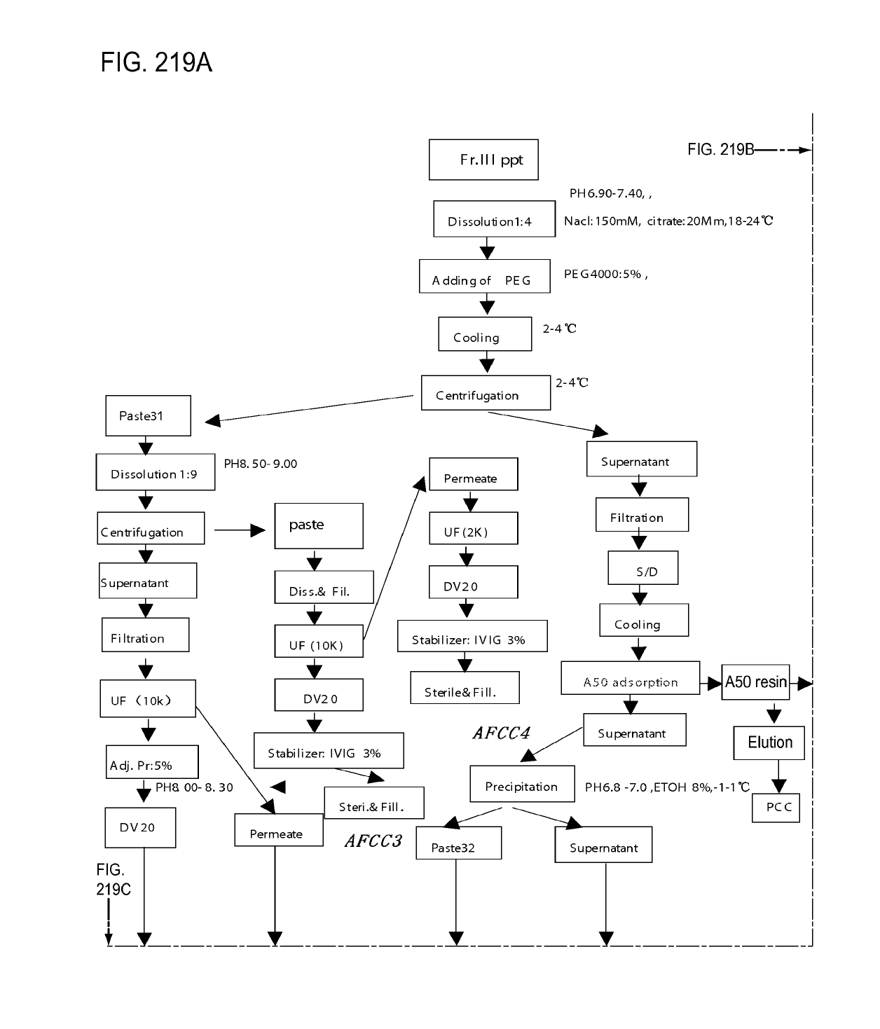

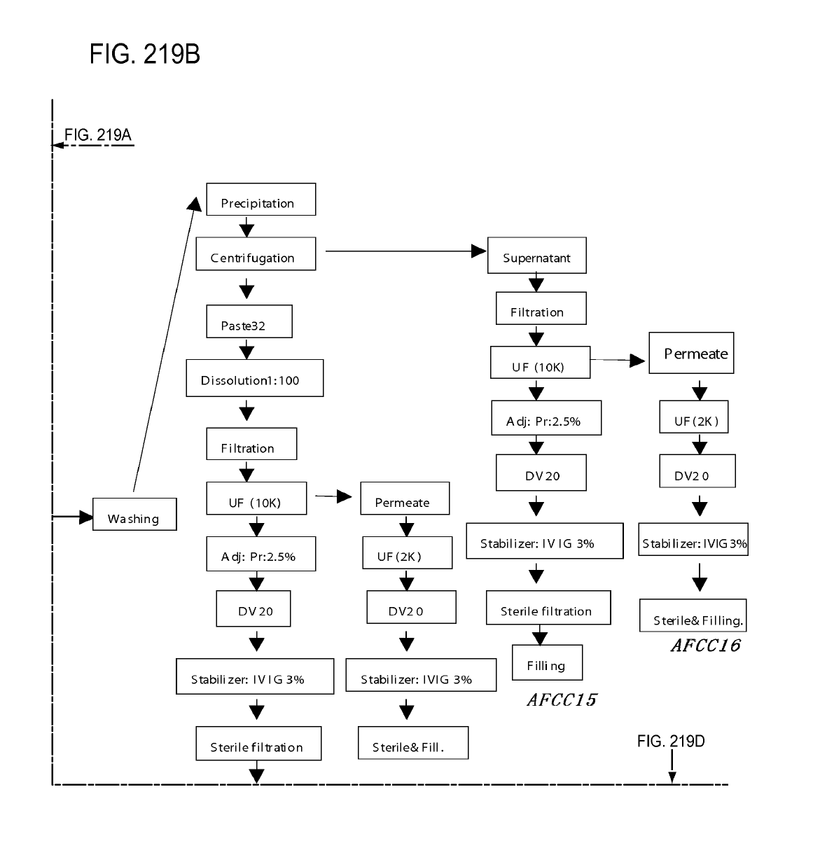

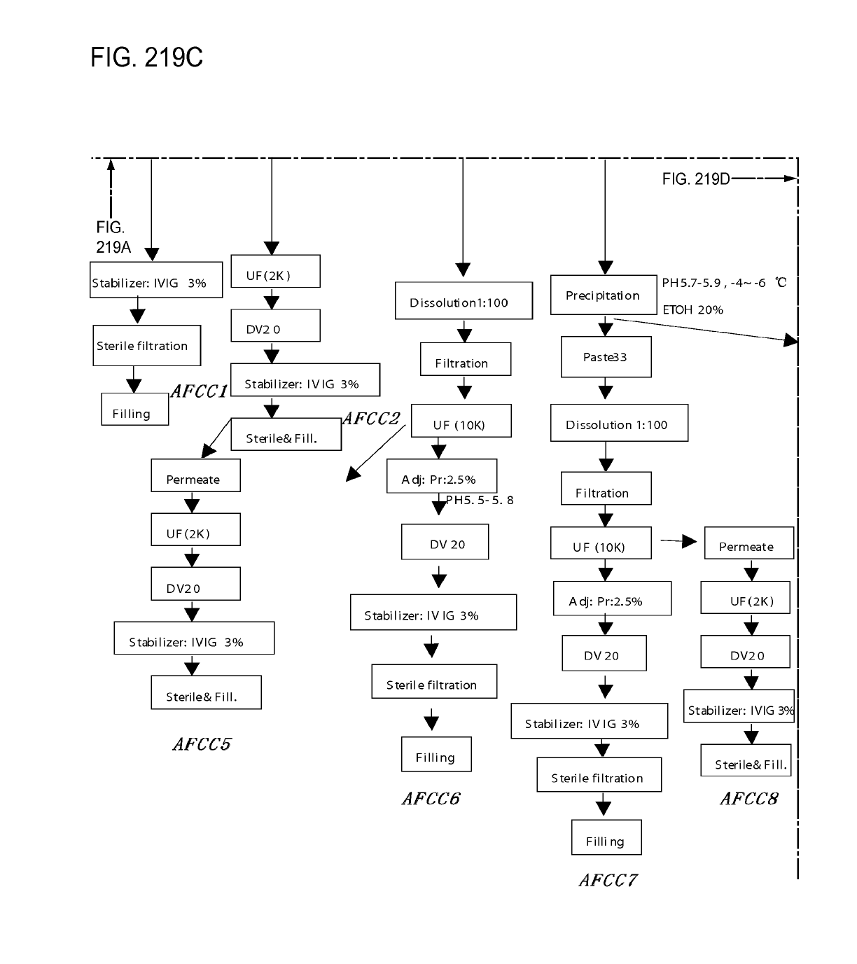

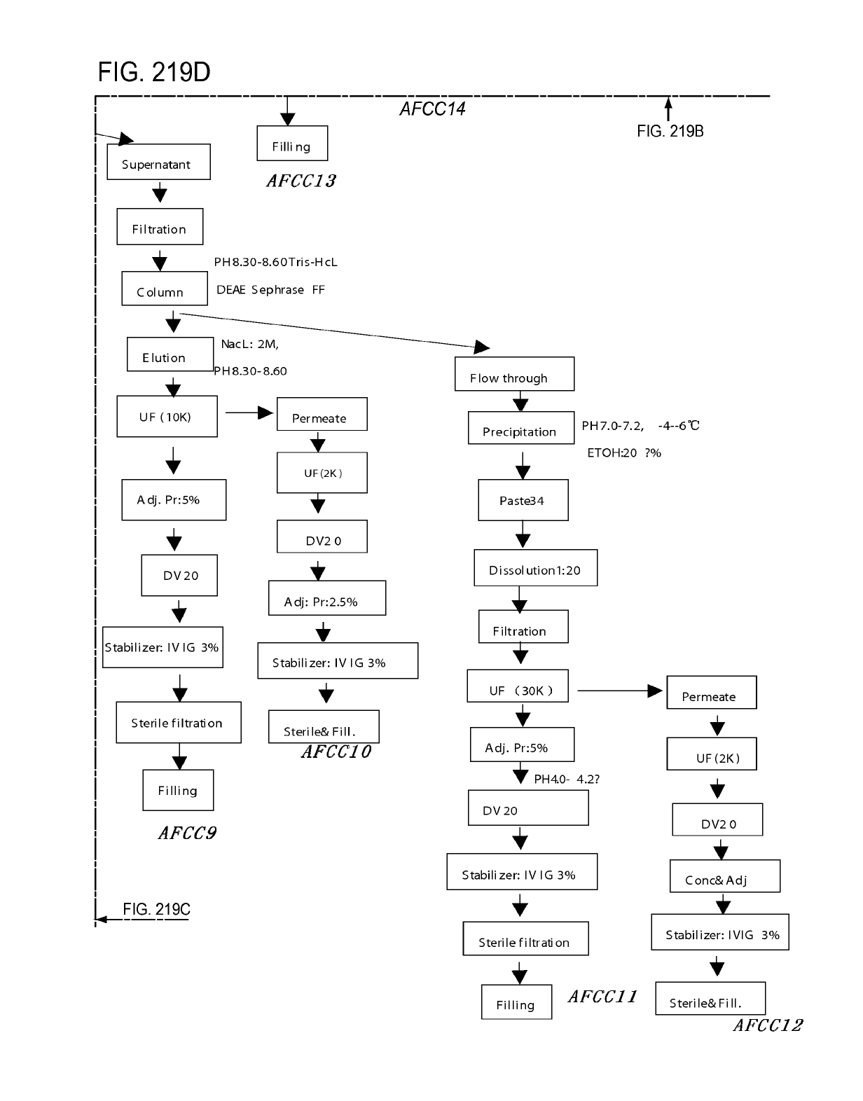

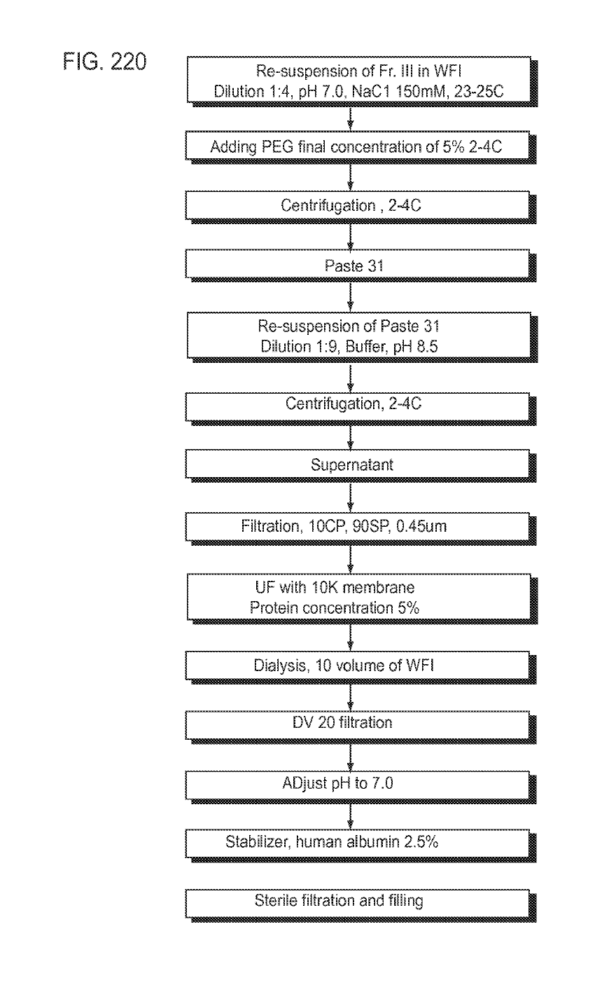

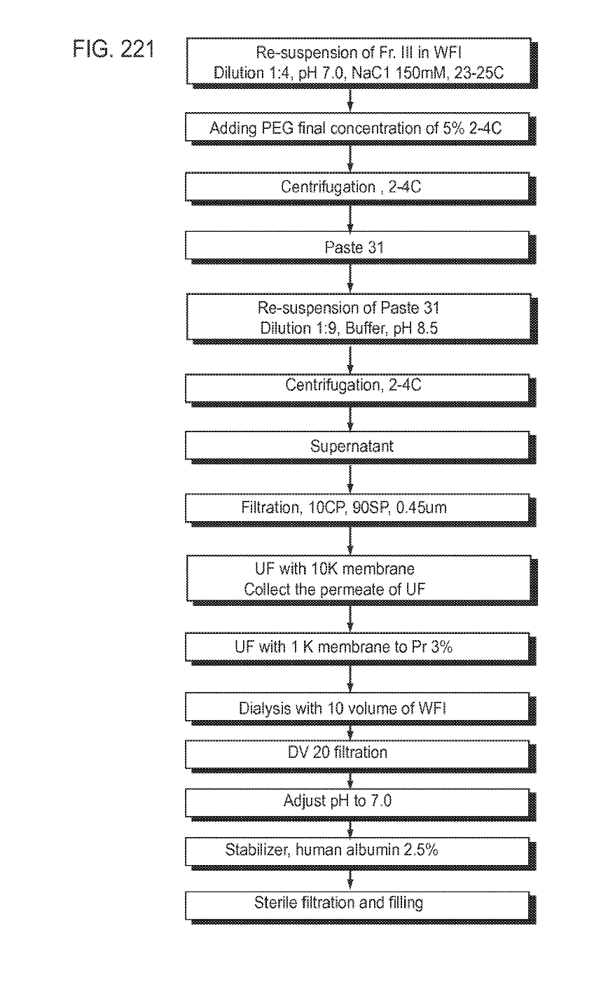

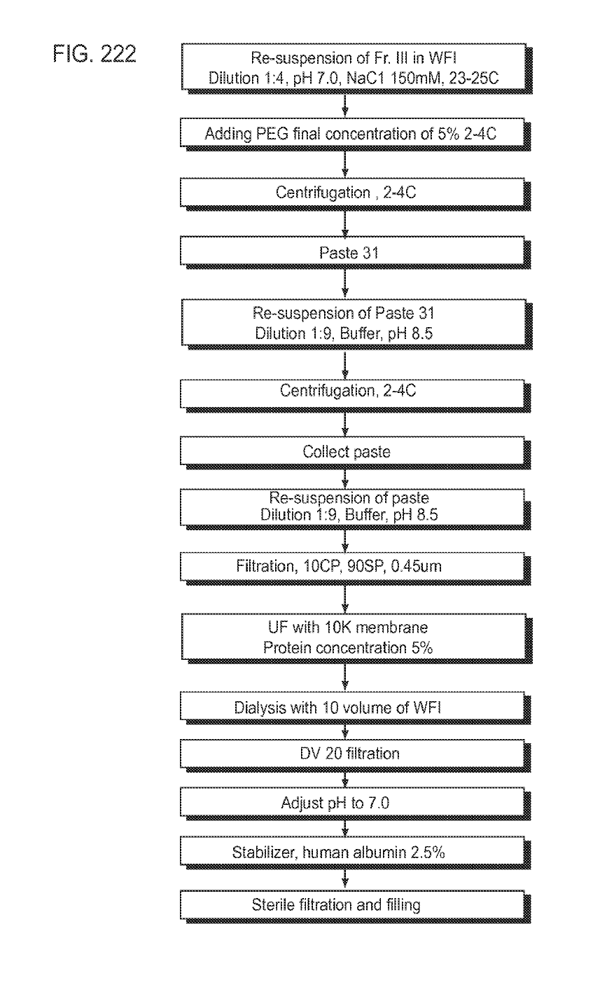

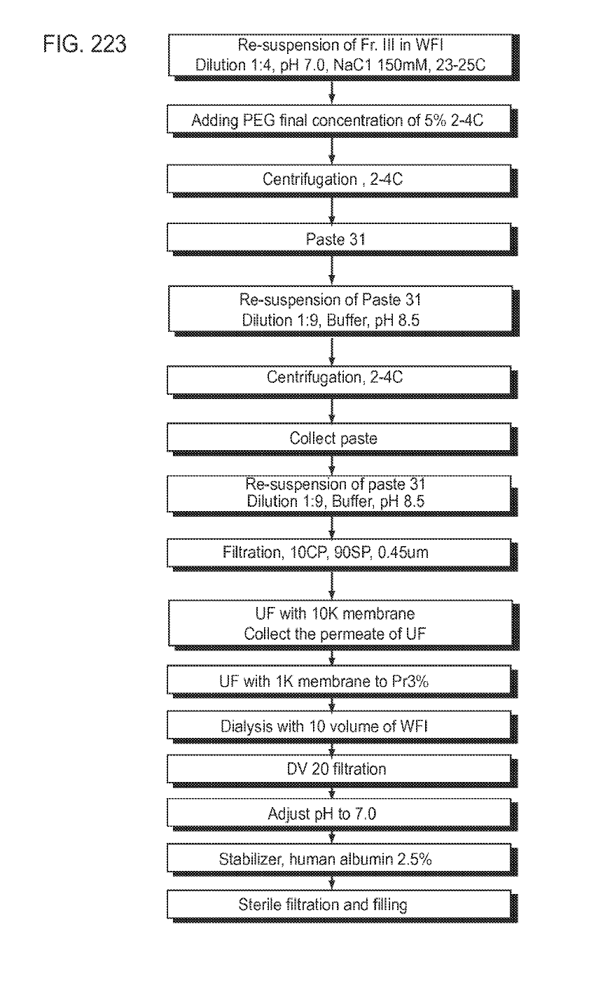

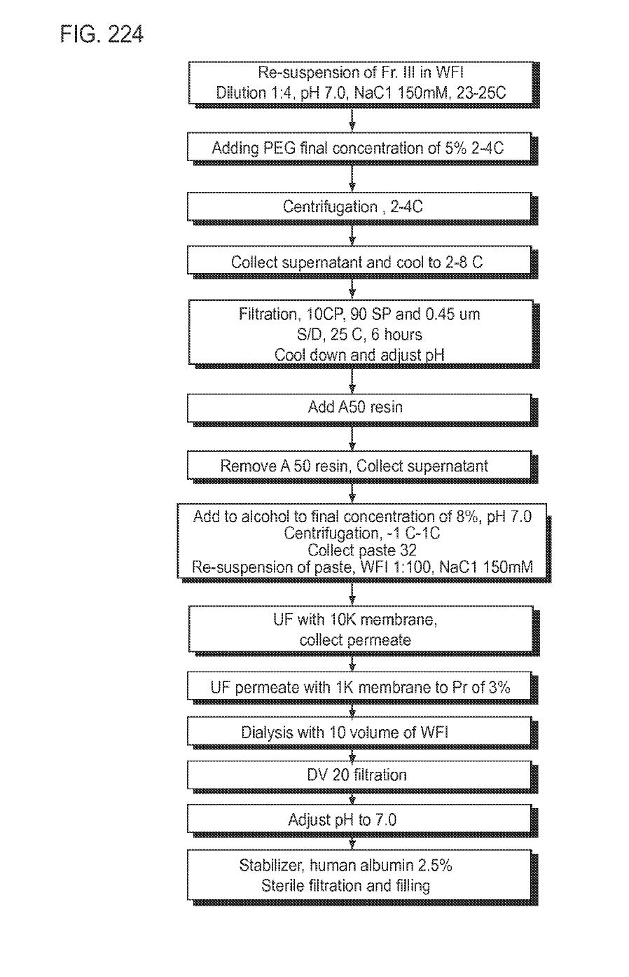

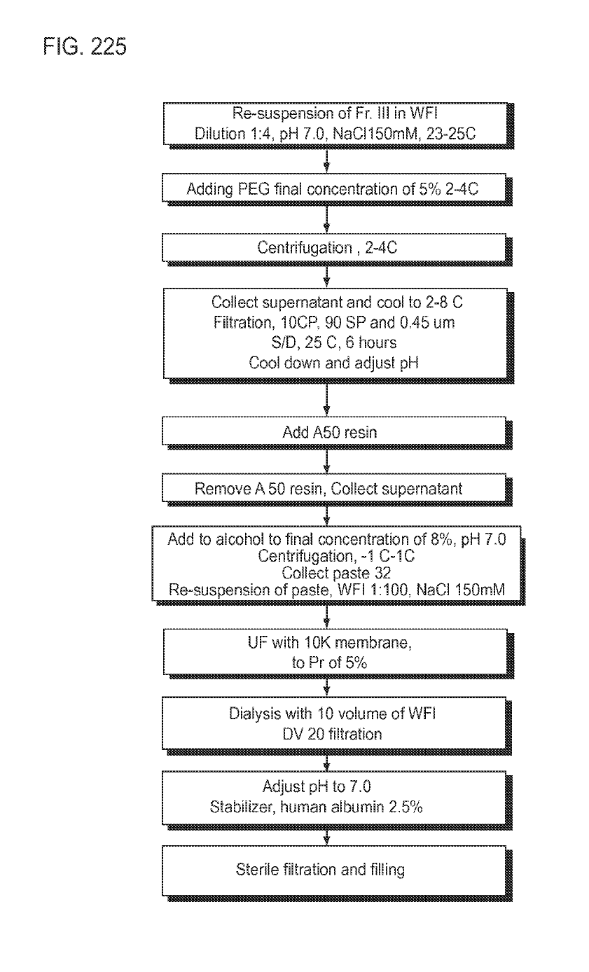

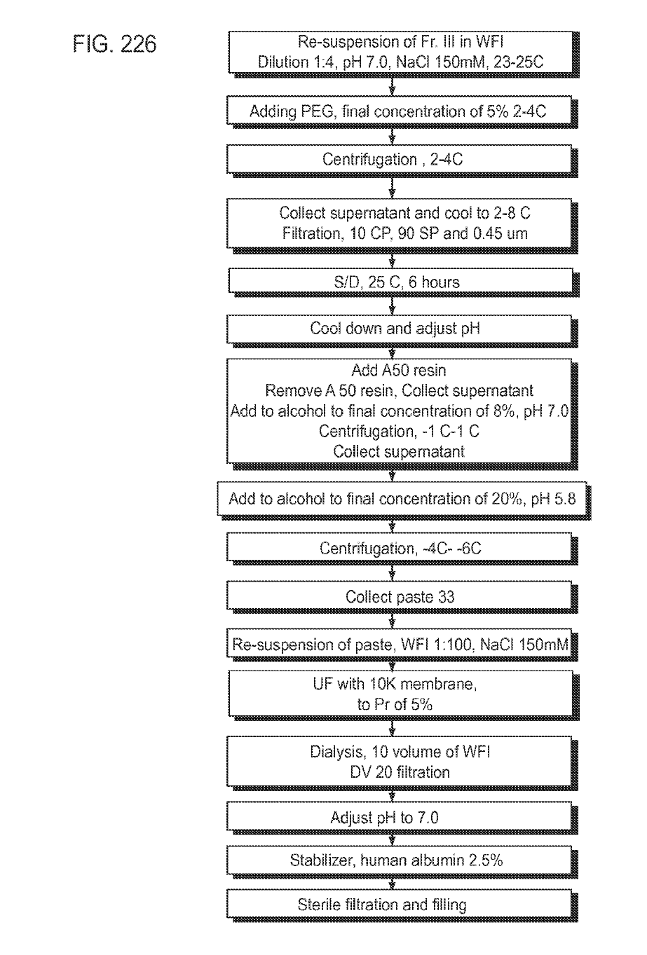

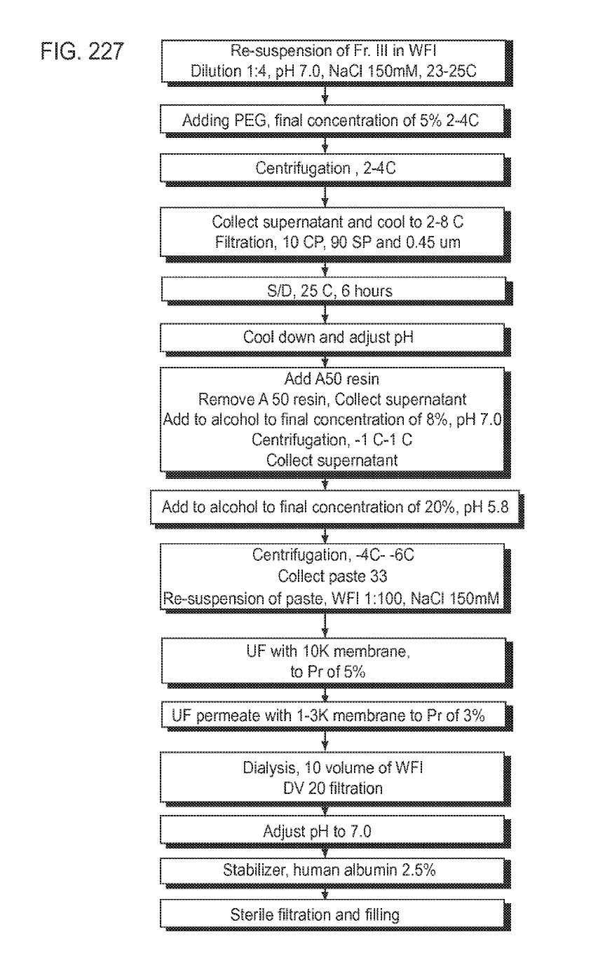

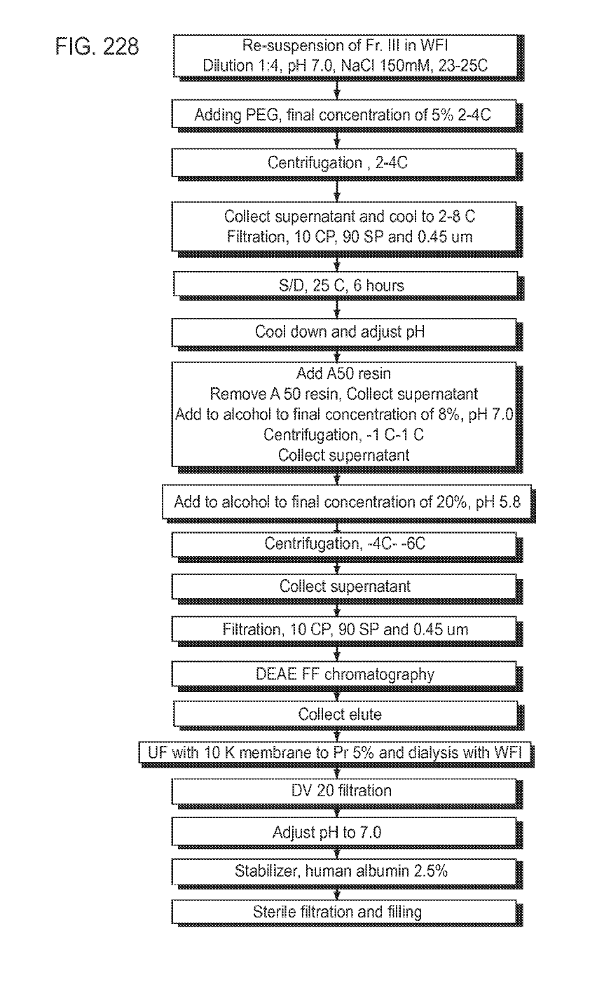

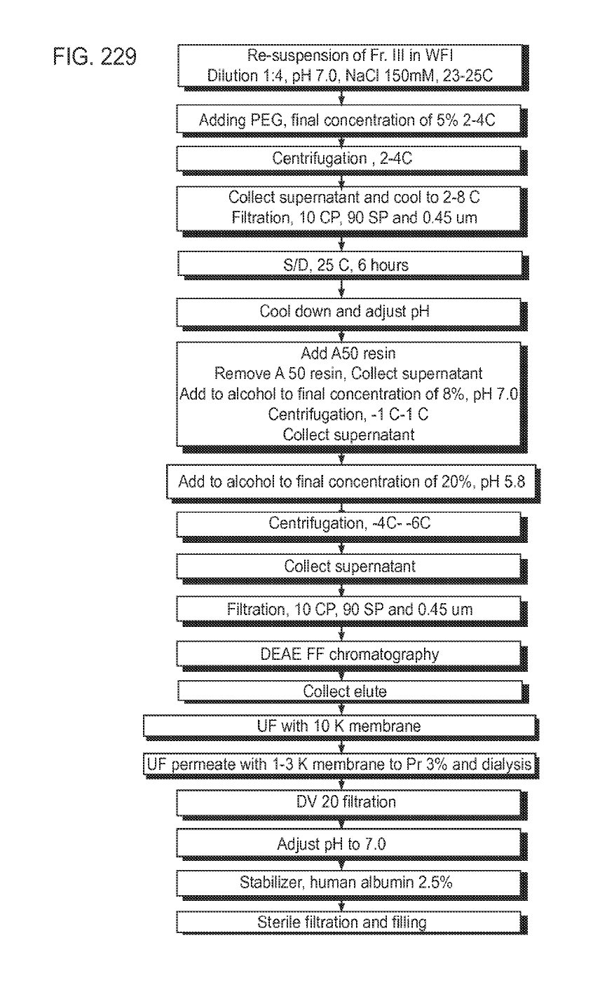

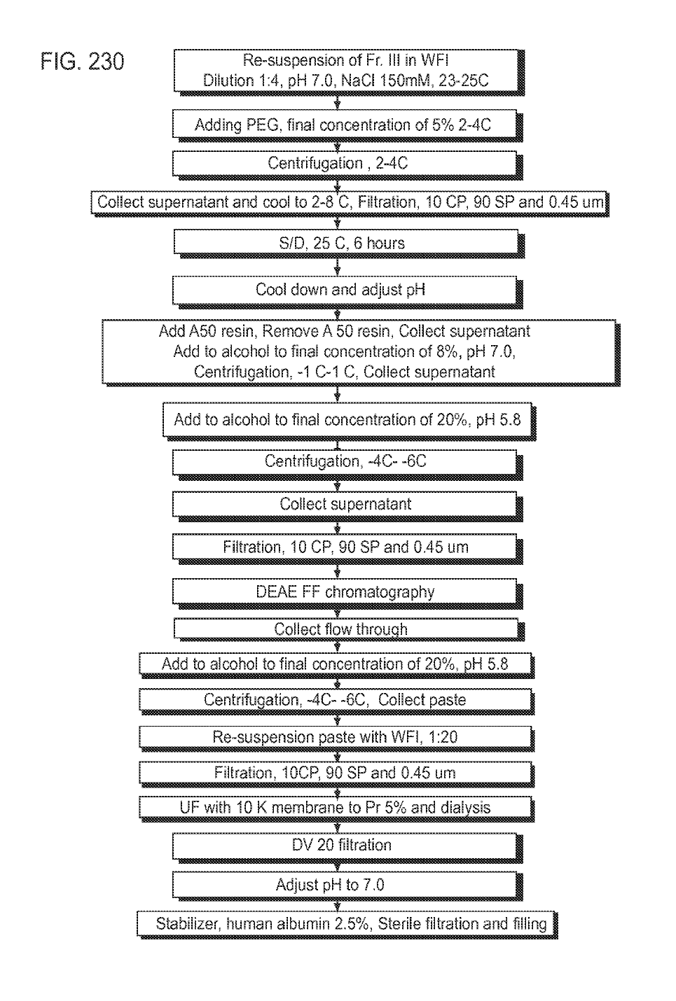

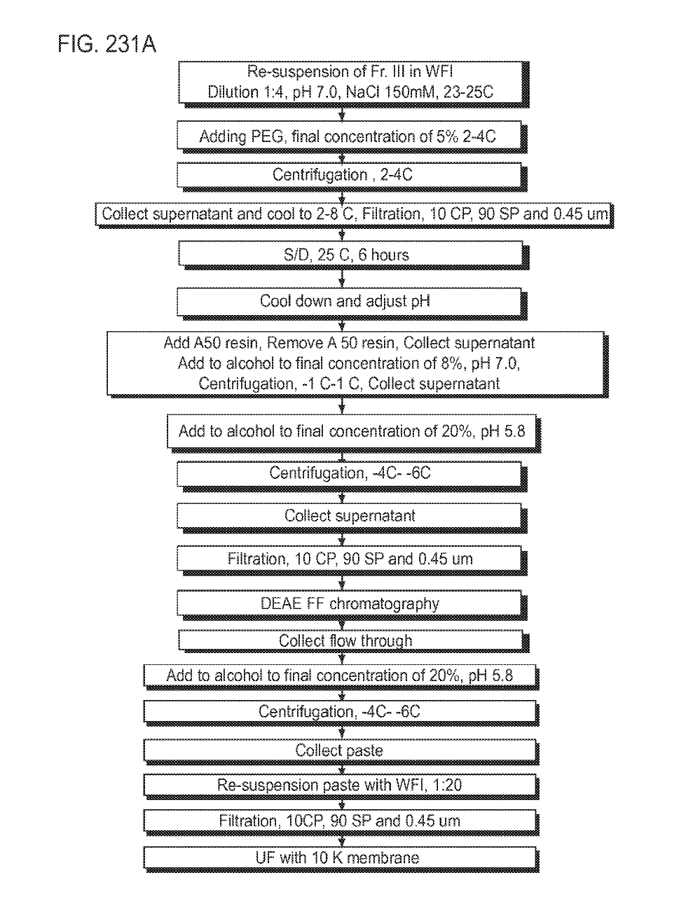

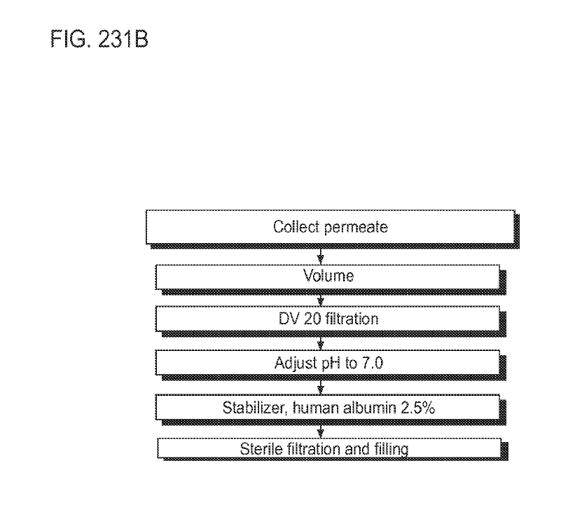

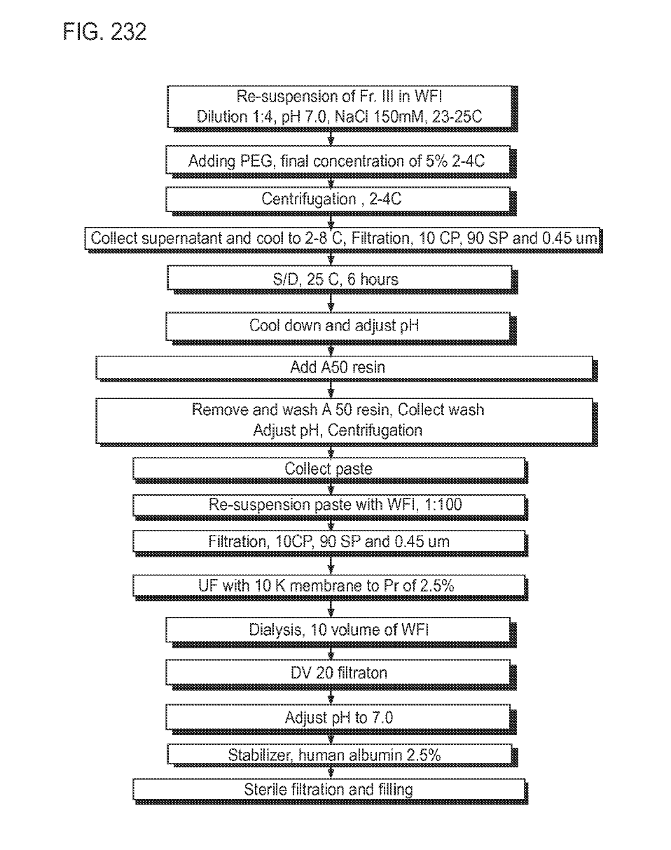

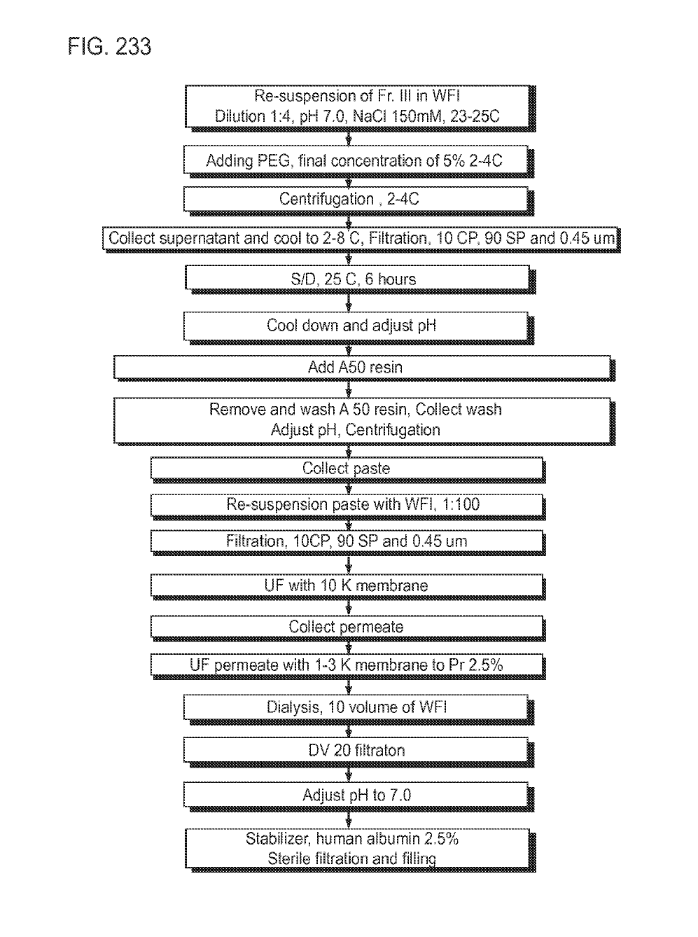

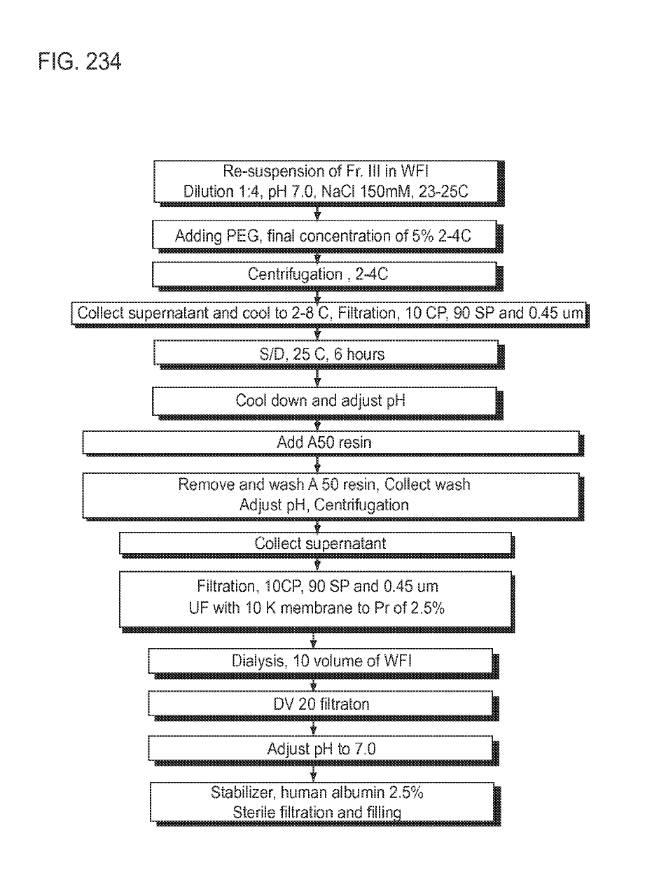

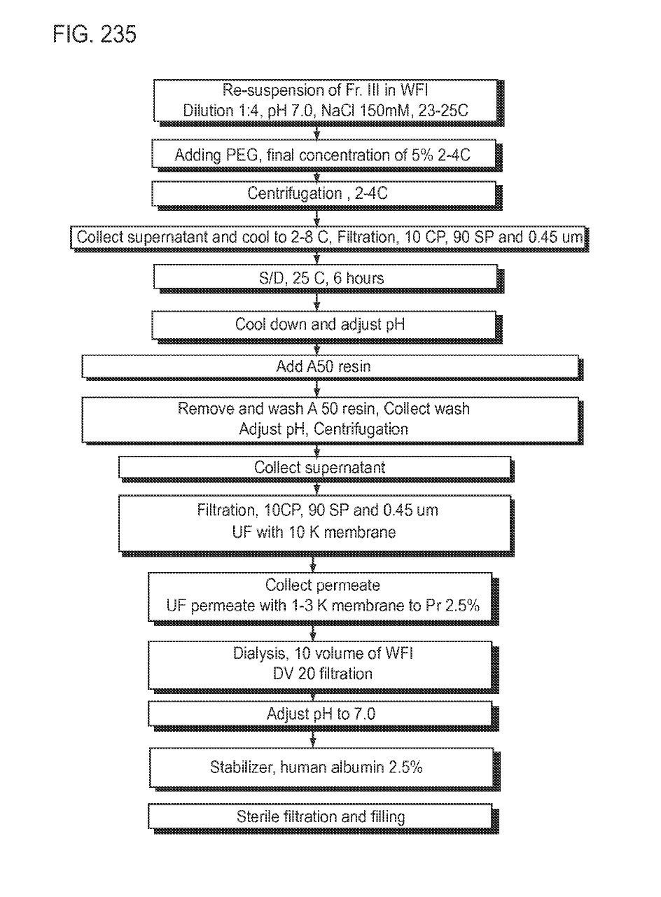

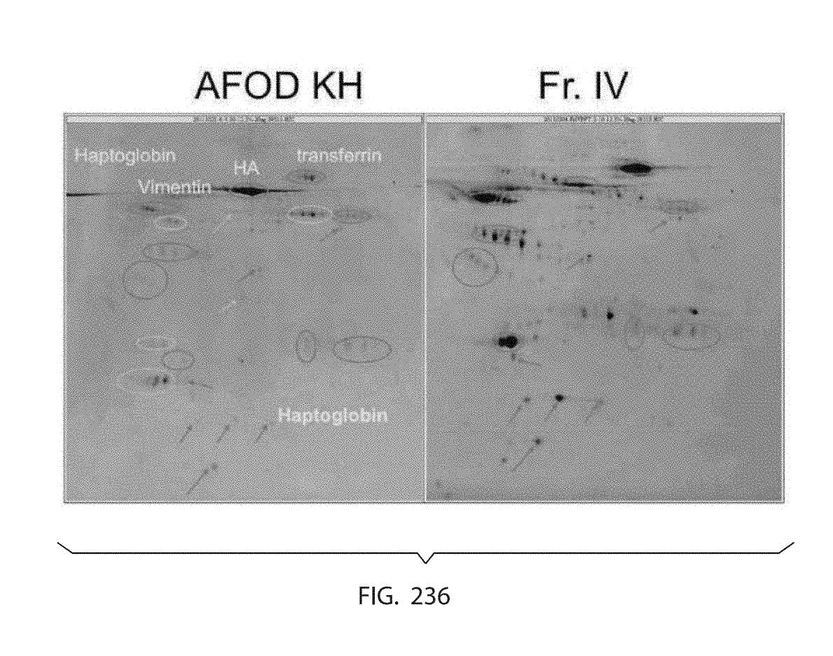

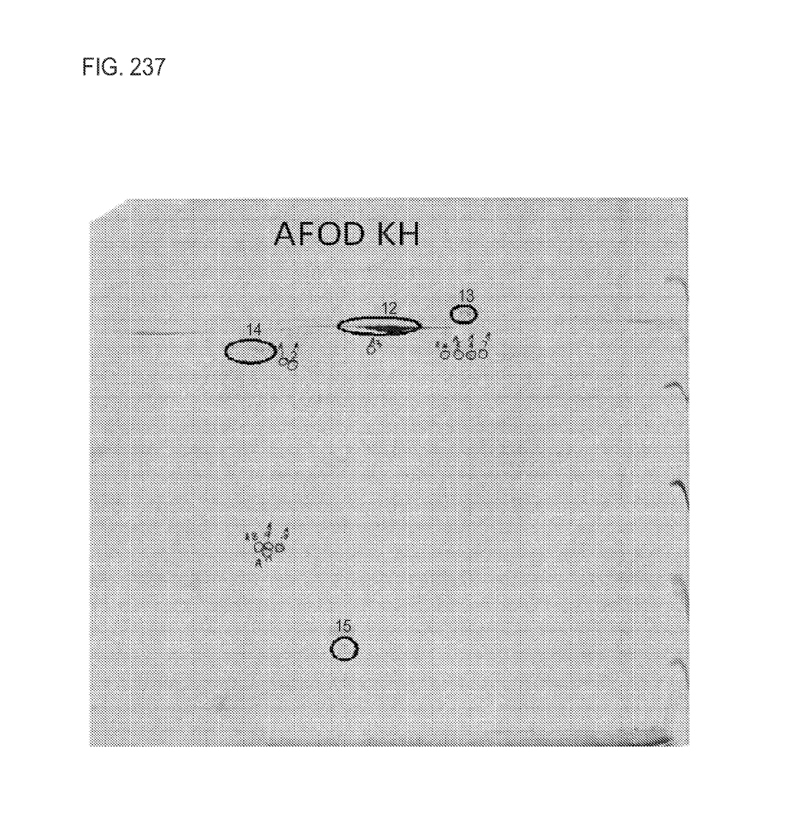

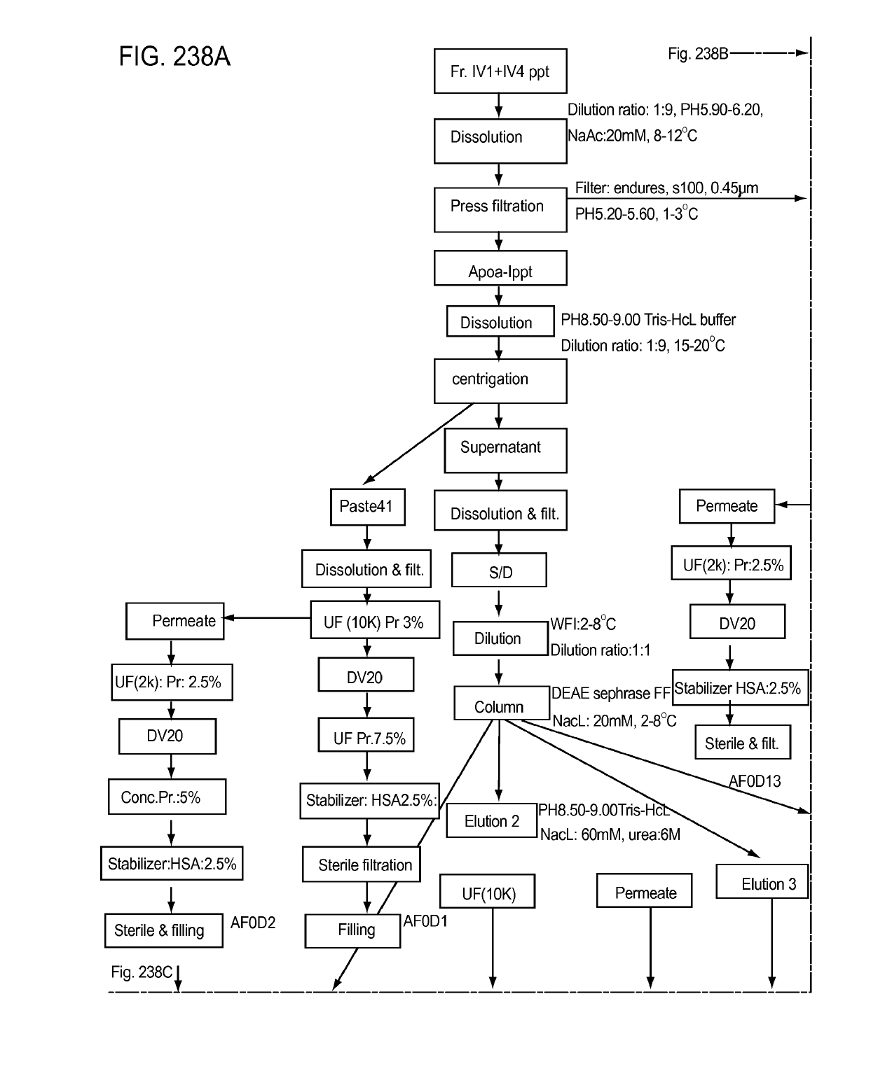

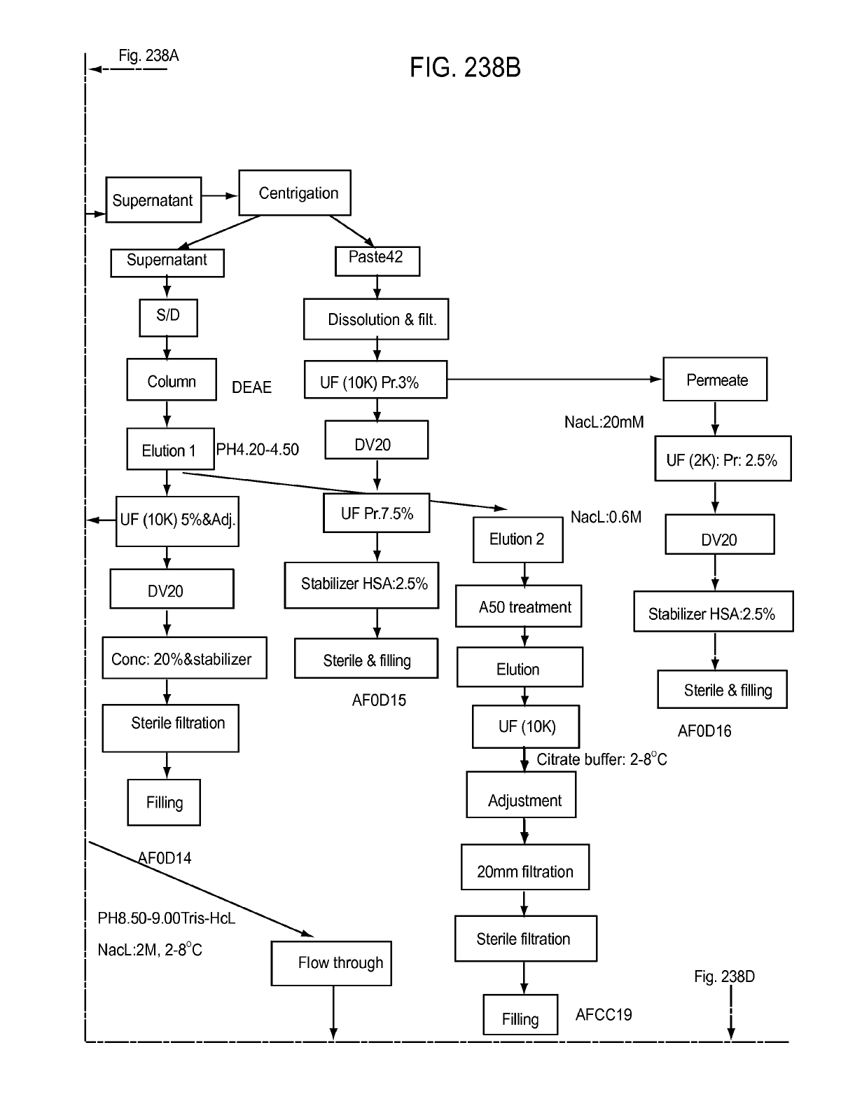

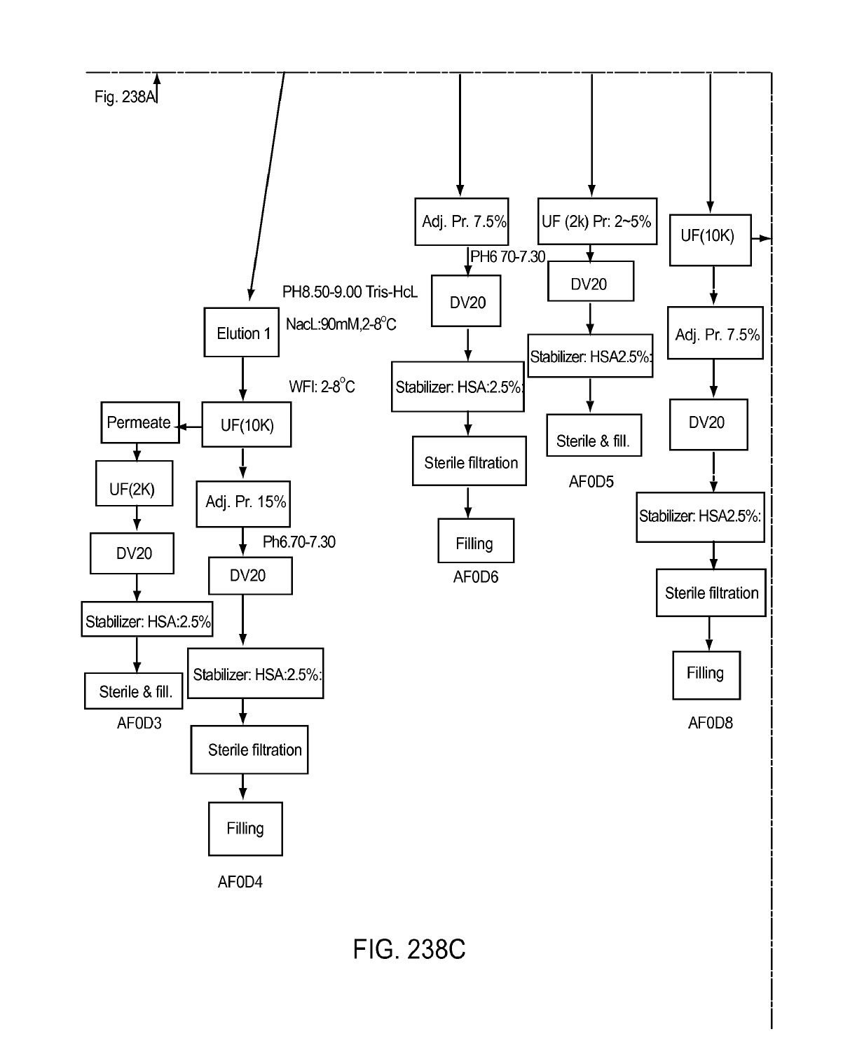

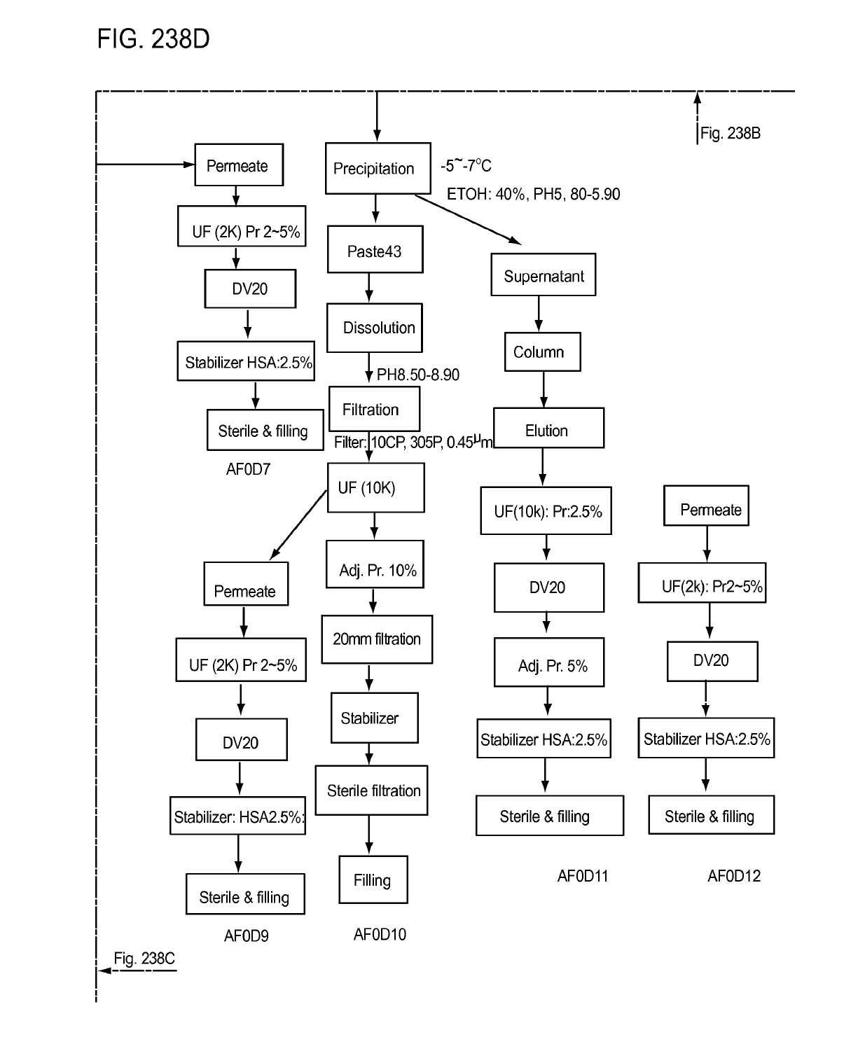

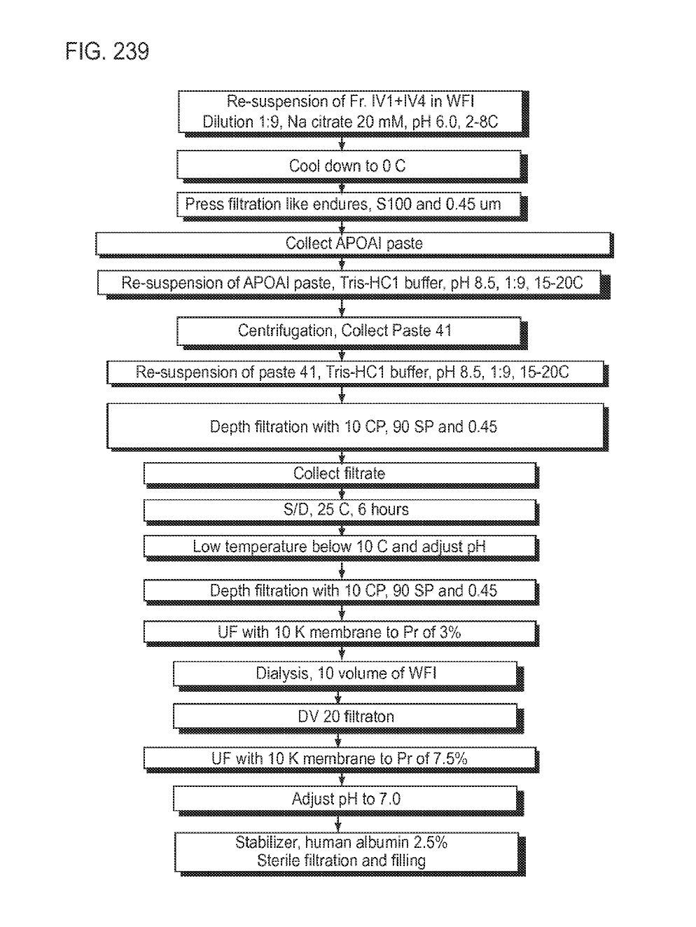

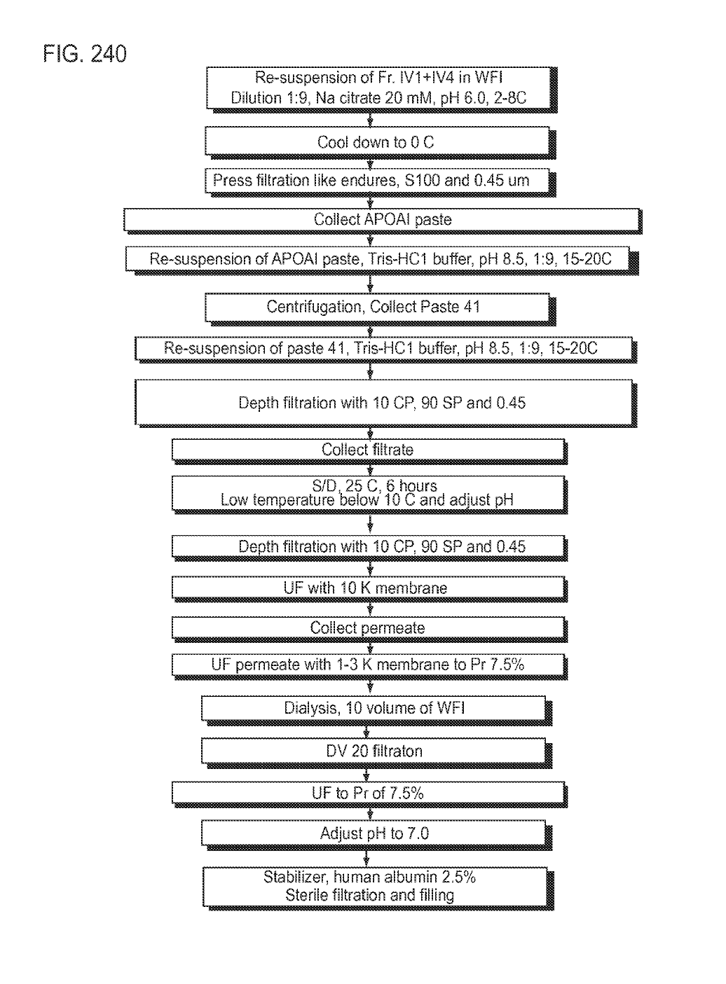

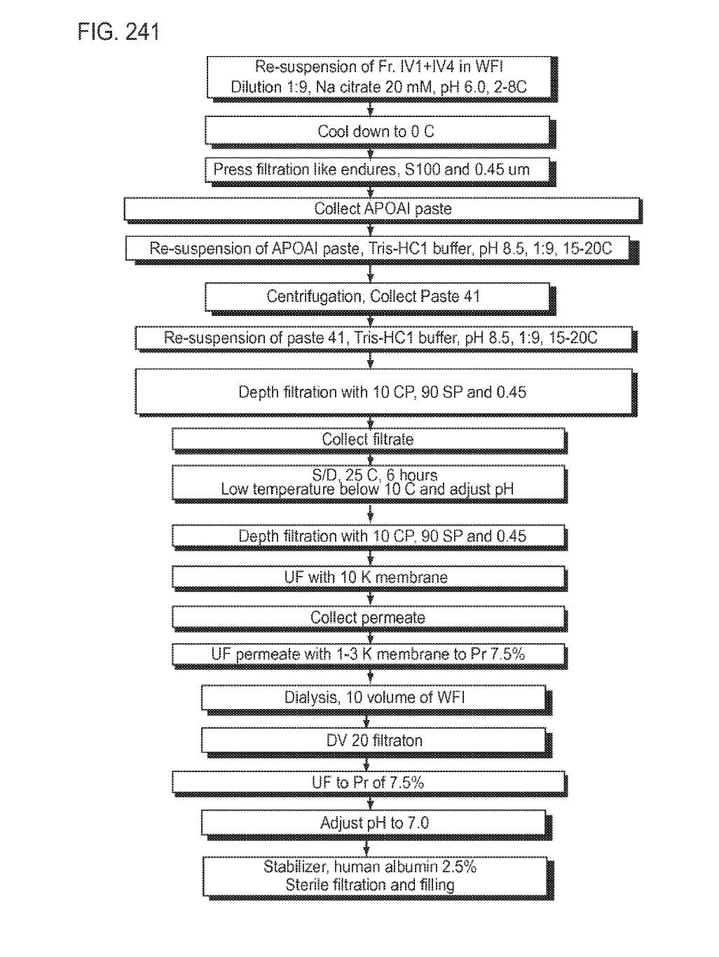

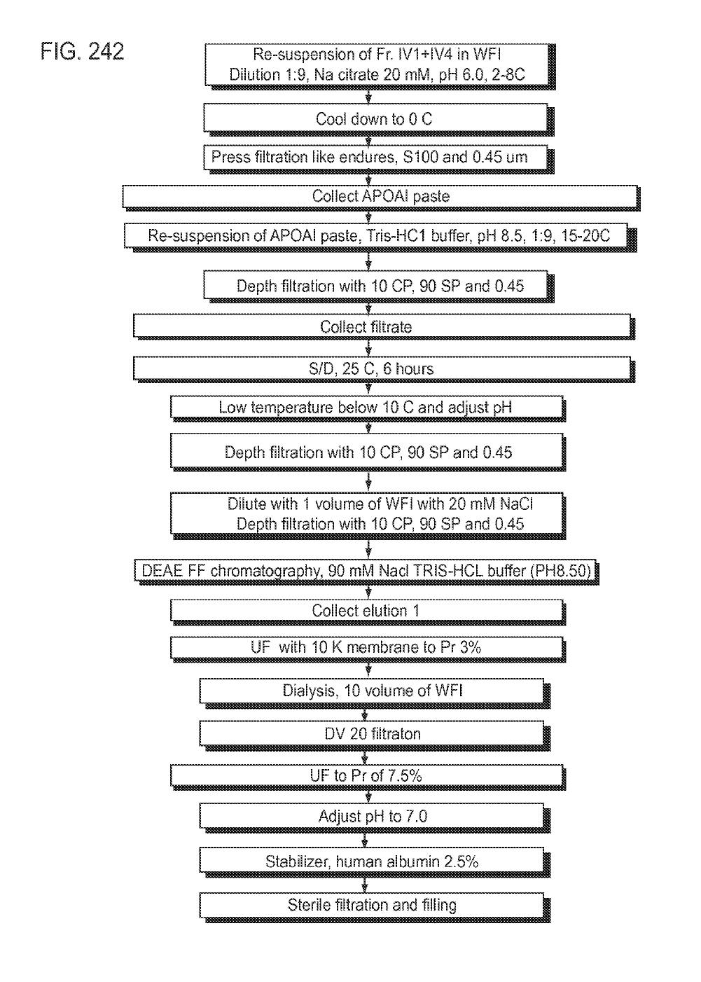

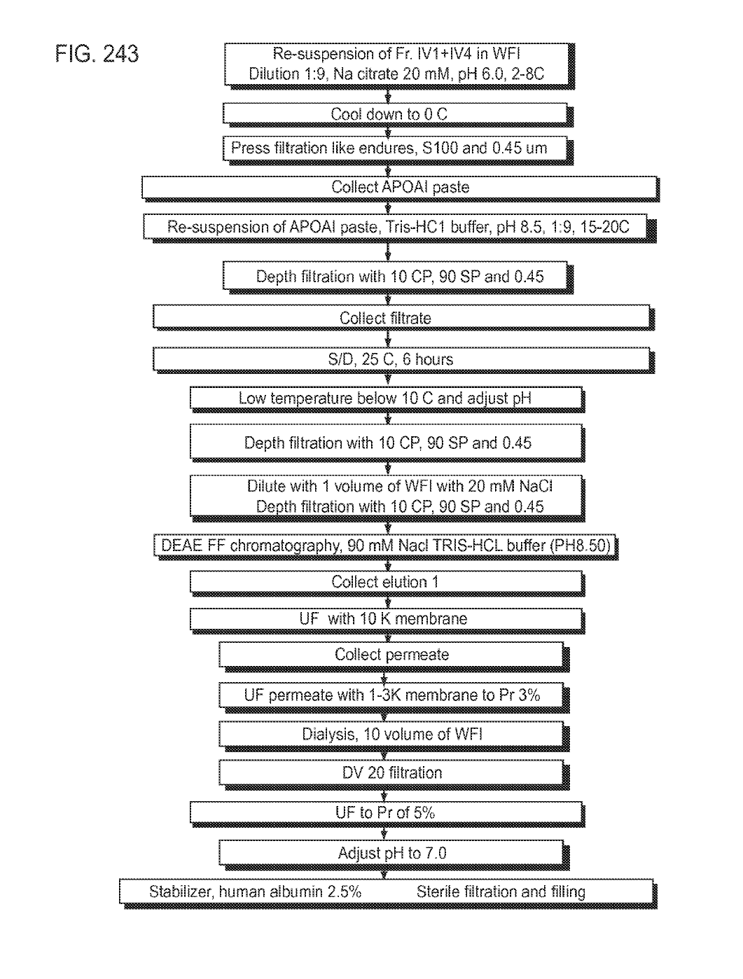

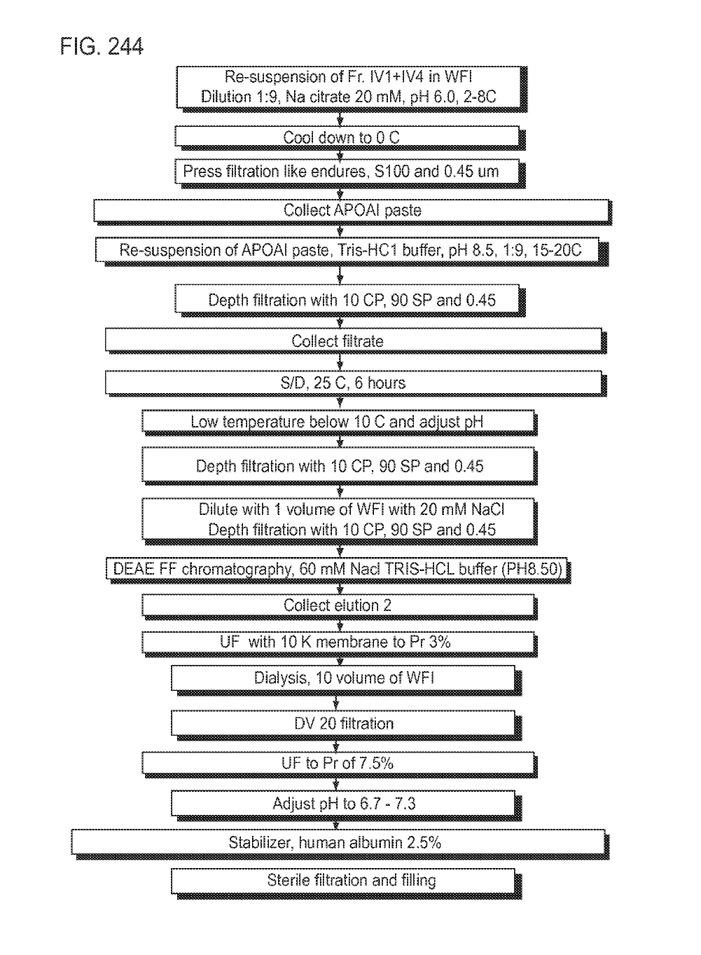

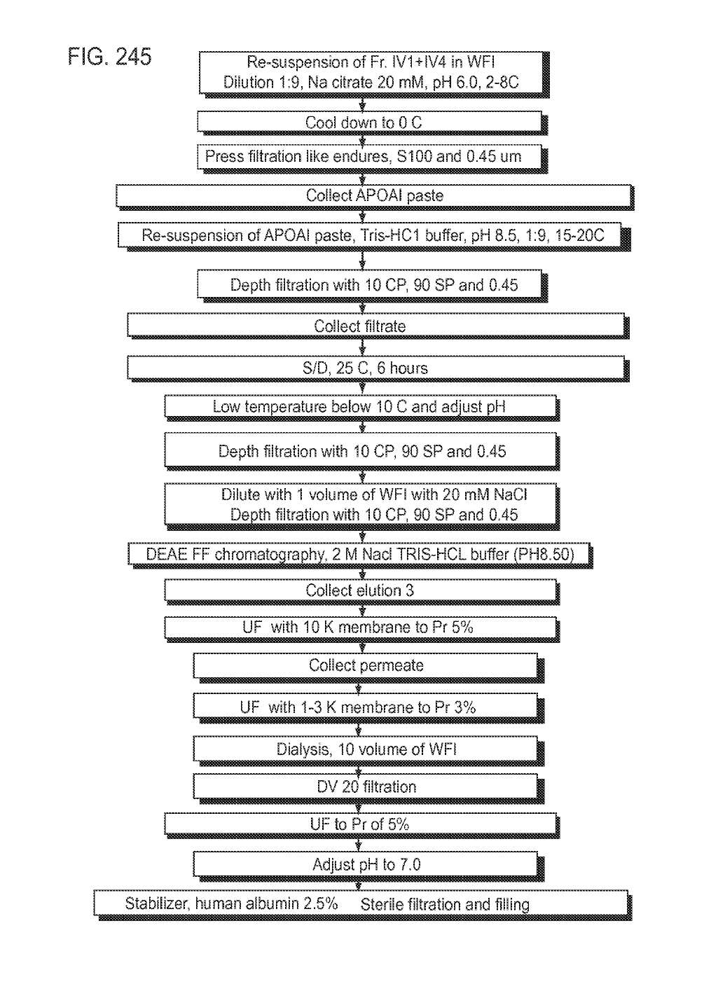

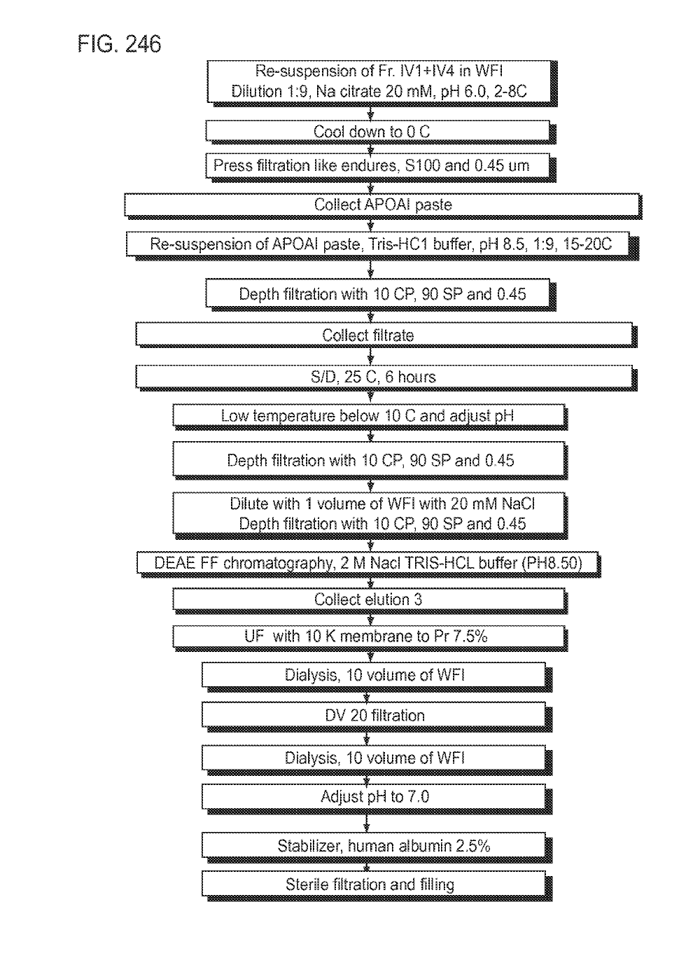

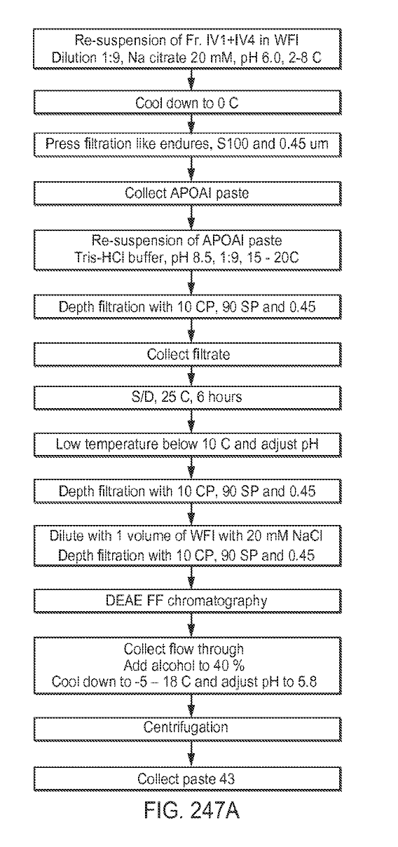

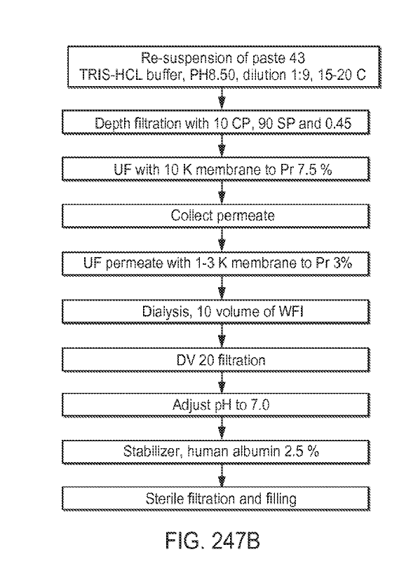

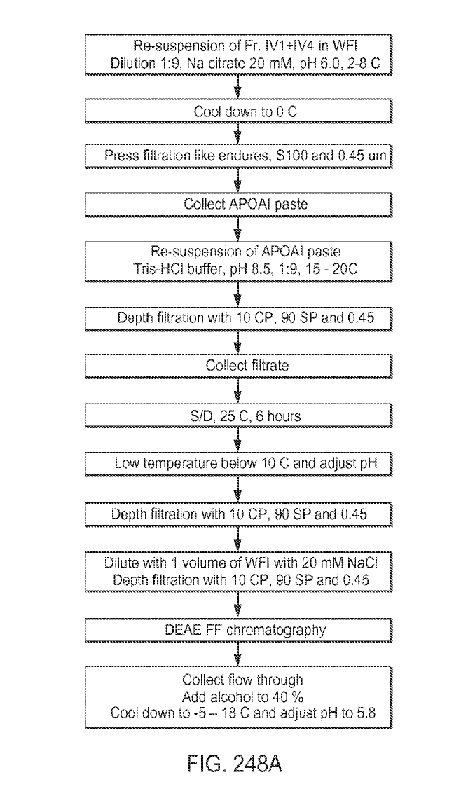

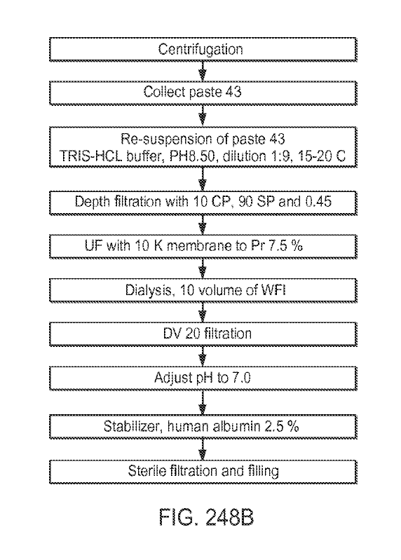

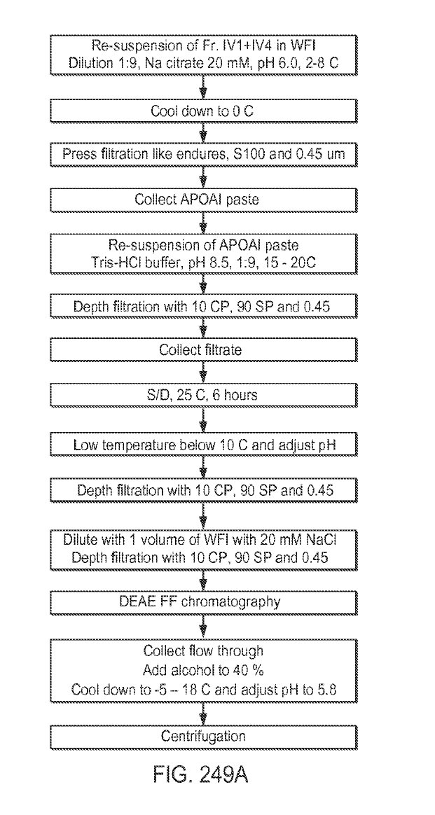

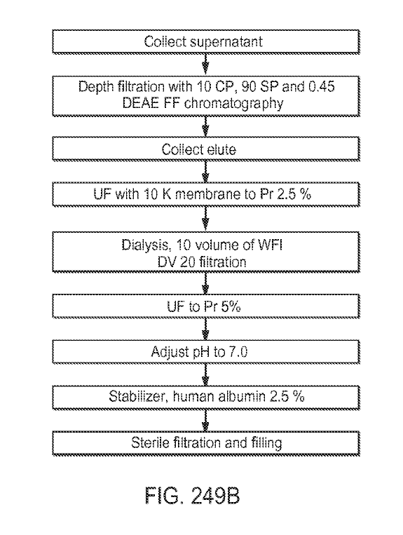

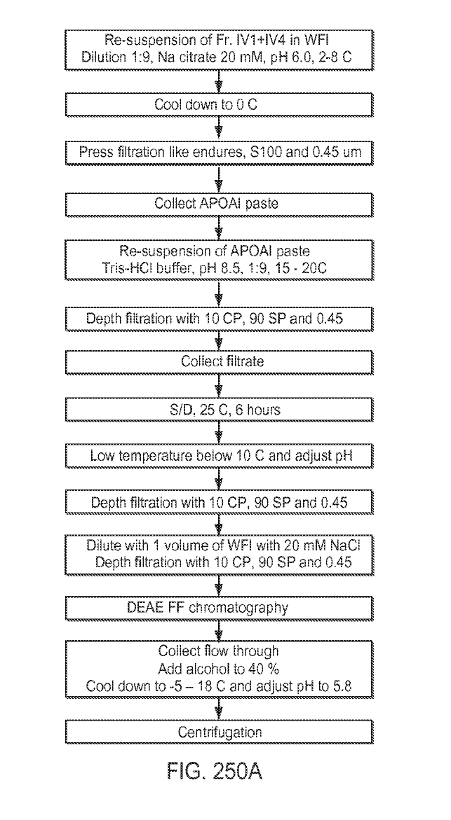

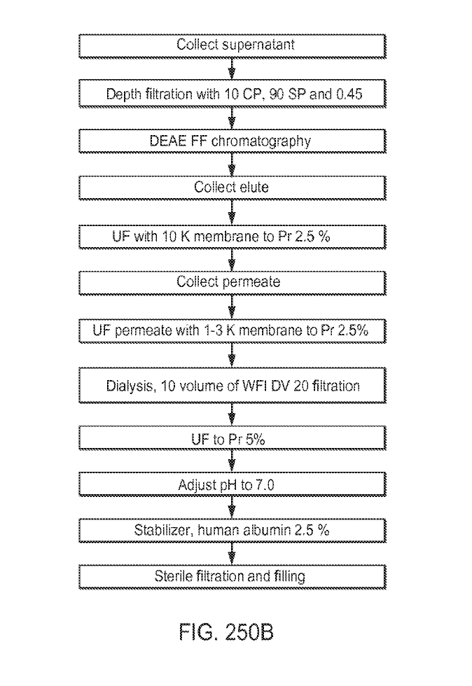

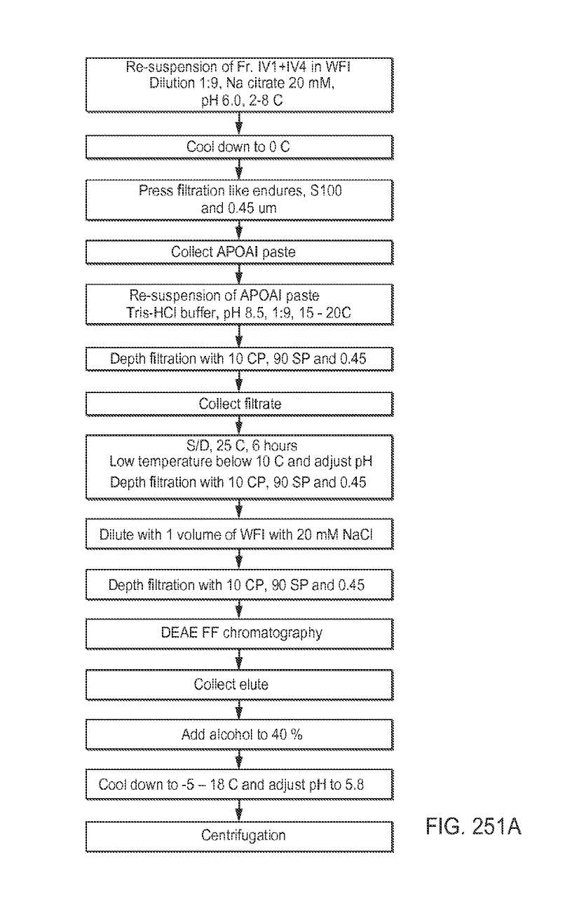

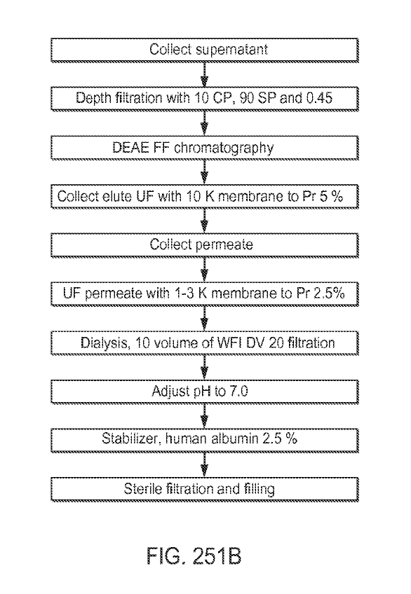

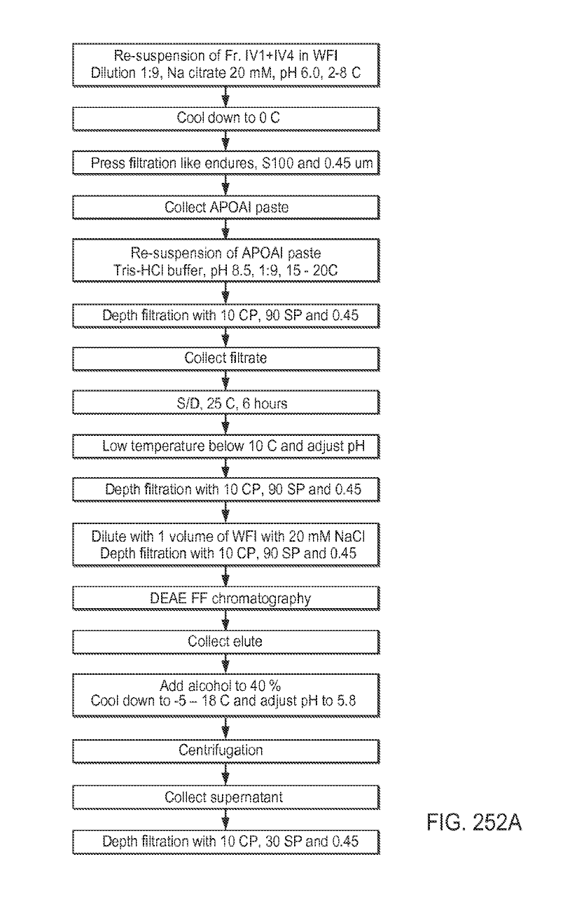

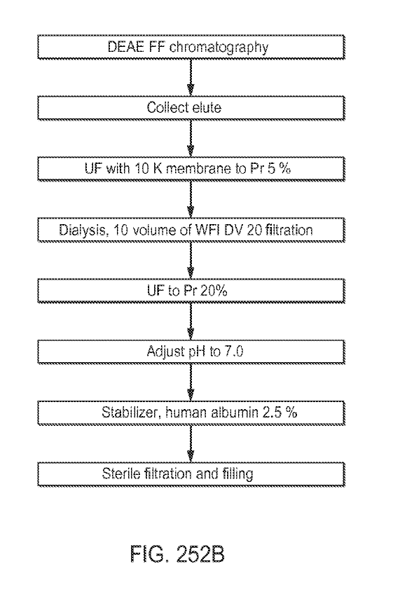

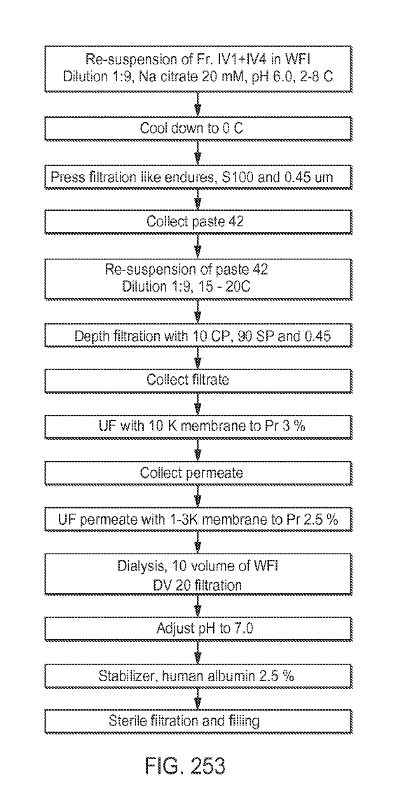

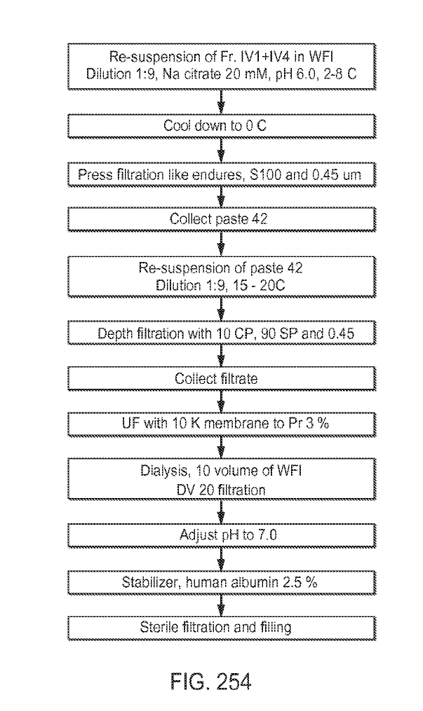

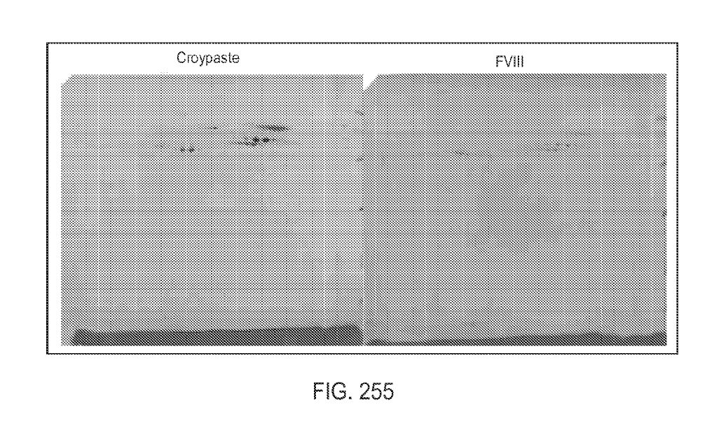

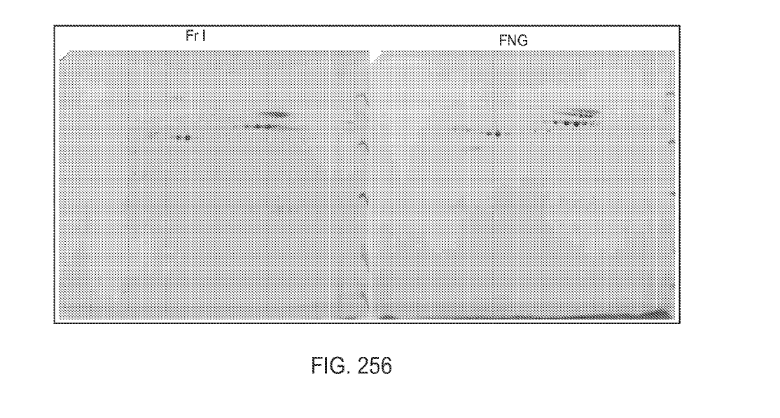

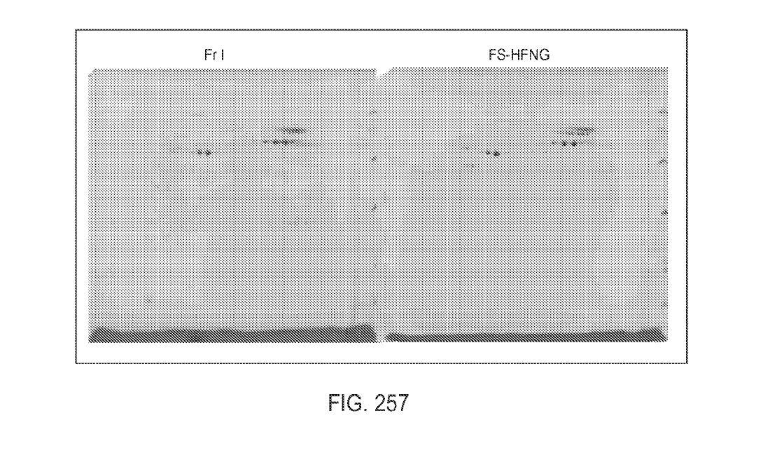

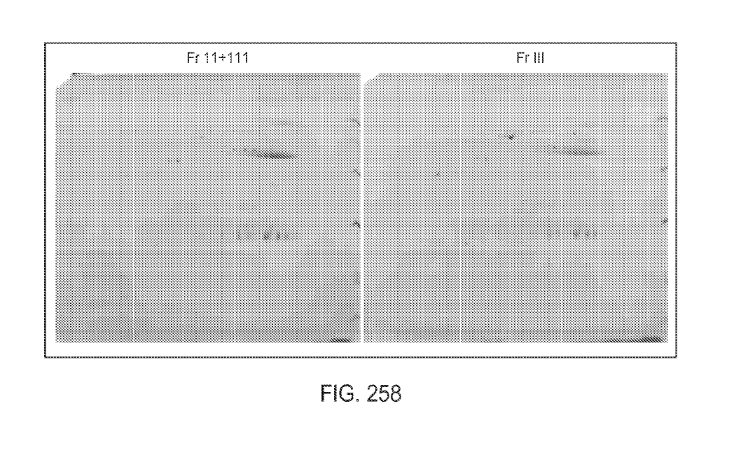

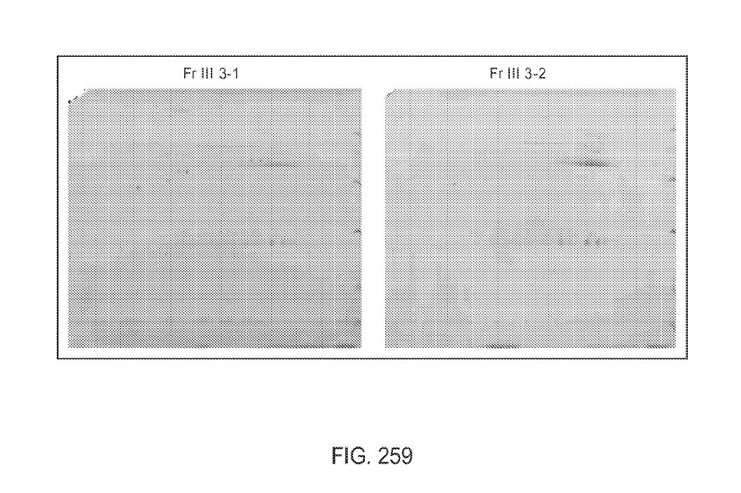

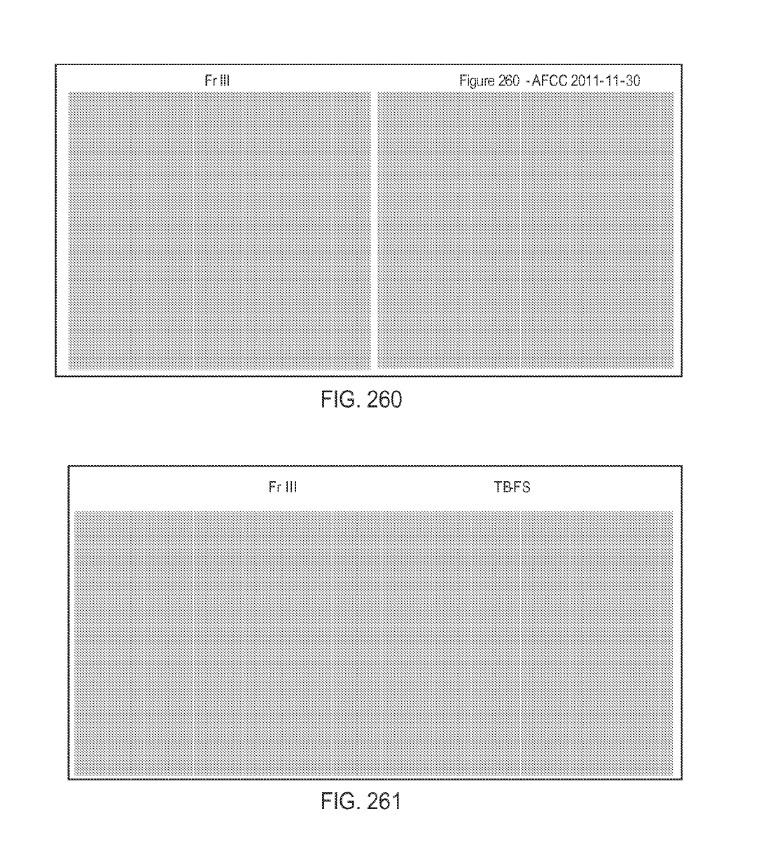

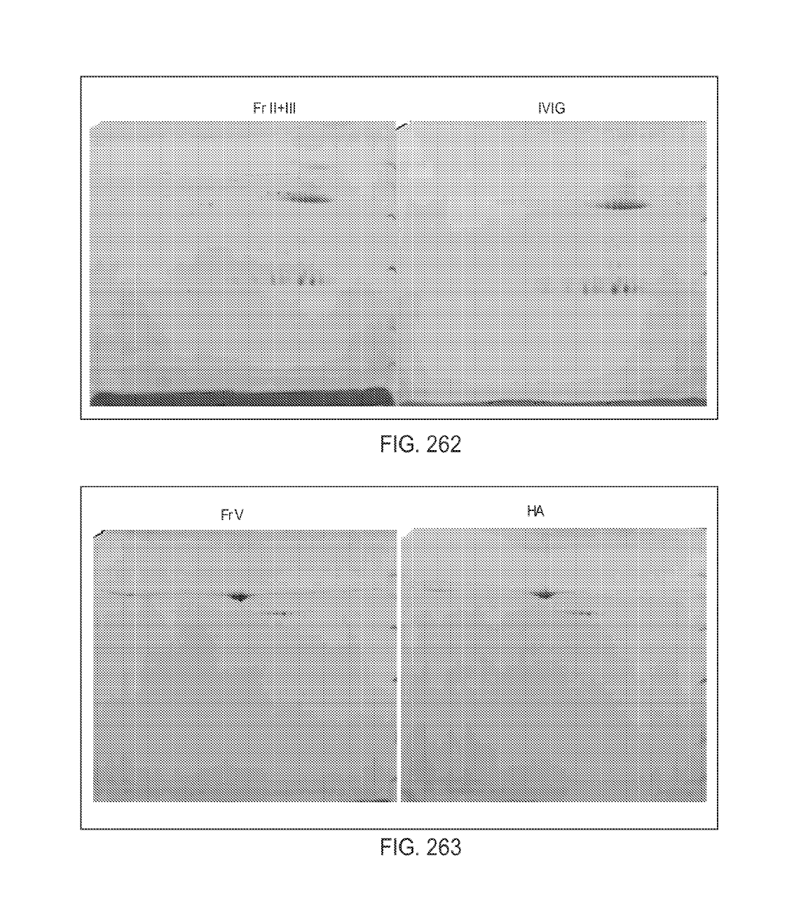

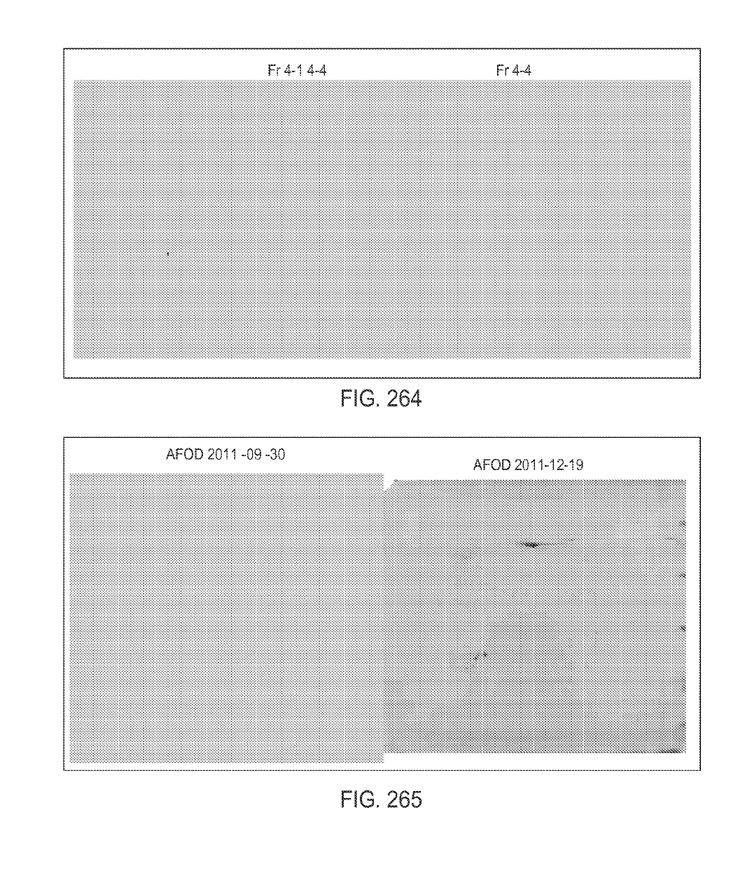

FIG. 45-1 through 45-18 Dose-dependent curves (EC50 values)

IV. Conclusions

e The Z factors of the cytotoxicity assay plates are 0.83(1a-plate!), 0.79(1a-plate2), 0.71(1b-plate1), 0.68(1b-plate2), 0.65(2a-plate1) and 0.83(2a-plate2), which are better than our QC standard.
The Z factors of the anti-replicon assay plates are 0.75 (1a-plate1), 0.70(1a-plate2),

0.87(1b-plate1), 0.75(1b-plate2), 0.58(2a-plate1) and 0.75(2a-plate2), which are better than our QC standard.
EC50 of the positive control Ribavirin in this study are 57.58 uM (1a), 39.04 uM (1b), and

:37.44 (2a), which are consistent with our previous data.

V. References
1. Mutations in Hepatitis C Virus RNAs Conferring Cell Culture Adaptation V. Lohmann et al., 2001 J. Virol.
2. Development of a replicon-based phenotypic assay for assessing the drug susceptibilities of HCV NS3 protease genes from clinical isolates. Qi X et al., Antiviral Res. 2009 February; 81(2:)166-73

IN Vitro Study—PCR Testing for HCV

| | undiluted |
|---|---|
| CT | 20.1 |
| Q | 2.98E+07 |

| Negative plasma | | | |
|---|---|---|---|
| [symbol] | 2.0 fold | 2.000 fold | Drug alone |
| CT | 2i .. 6 | 3o.e | N |
| Q | 2.55E+06 | 1.69E+04 | N |

| Drug dilution | 20 fold | 2000 foid | Drug alone |
|---|---|---|---|
| CT | 25.B | 3′1 ., | N |
| Q | 5.62E+05 | i .37E+04 | N |

[symbol]

indicates data missing or illegible when filed

Results: after 10 days incubation of samples diluted on 2012 Jun. 1 at 4 C refrigerators, the test was conducted again. It showed that Ct value was 2 Ct advanced in negative plasma than in drug diluted at

20 fold dilution. There is no difference at 2.000 fold dilution.

IN Vitro Study—PCR Testing for HIV

| | undiluted |
|---|---|
| CT | |
| | 2.30E+07 |

| Negative plasma | | | |
|---|---|---|---|
| [symbol] | 20 fold | 2000 fold | Drug alone |
| CT | 23.. 9 | 30.. 3 | N |
| 0 | 2.1.1E+06 | 2.32E+04 | N |

| Drug dilution | 20 fold | 2000 foid | Drug alone |
|---|---|---|---|
| CT | 2?.B | N | N |
| () | •1.34E+05 | N | N |

[symbol]

indicates data missing or illegible when filed

Results: after 10 days incubation of HIV samples diluted on 2012 Jun. 1 at 4 C refrigerators, the test was conducted again. It showed that Ct value was 4 Ct advanced in negative plasma than in drug diluted at

20 fold dilution. There is no detection at 2.000 fold dilution of drug dilution.

IN Vitro Study—PCR Testing for HBV

| | undiluted |
|---|---|
| CT | 27.91 27.7 |
| CT mean | 27.8 |
| Q | 1.21E+03 |
| | 11.38E+0 [symbol] |
| Qmean | 1.29E+03 |

| Drug dilution | 2 fold | 10 fold | Drug alone |
|---|---|---|---|
| CT | L.,.a_,:-f; | | |
| I | 29.9 | 30.61 N | N |
| | ; 9. ? | | |
| [symbol] | 3.84E+02 | 1.94E+021 N | N |
| | 13.15E+02 | | |
| Qmean | 3.. JOE+02 | 1.9.:-lE+02 | N |

| Negative plasma | | | Negative plasma |
|---|---|---|---|
| dilution | 2. fold | 10 fold | alone |
| CT | 2.9.31 28.6 | 32.51 30.4 | N |
| CT mean | 2B.9 | 31.5 | N |
| | 4.62E+02 | 5.2.9E+01 | N |
| | 17.56E+02 | 12.18E+02 | |
| Qmean | 6.09E+02 | .1 ,3. )[+0? | N |

[symbol]

indicates data missing or illegible when filed

Results: AFOD RAAS 104® (formerly AFOD RAAS 8) was diluted for 10 fold with normal saline and then the HBV positive plasma (1000) was diluted by this to 500 (2 fold dilution) and 100 (10 fold dilution). Negative plasma was also used as diluents for negative control. The CT value of 2 fold negative plasma diluted sample was 1CT advanced drug diluted. One of the duplicate in drug 10 fold dilution didn't detect virus. 10 fold dilution of negative plasma was not consistent in duplication.

Samples were kept at 4 C refrigerator for 3 days, 2012 Jun. 5

| | undiluted |
|---|---|
| CT | 28.51 28.3 |
| CT mean | 28.4 |
| Q | 9.46E+0211.10E+03 |
| Qmean | 1.02E+03 |

| Drug | 2 fold | 10 fold | Drug alone |
|---|---|---|---|
| CT | 30.21 31.0 | 3131 31.7 | N |
| CT mean | 30.6 | 31.S | N |
| Q | 3.04E+0211.72E+02 | 1.42E+0211.07E+02 | N |
| Qmean | 2.3SE+02 | | N |

| Negative plasma | | | Negative plasma |
|---|---|---|---|
| dilution | 2 fold | 10 fold | alone |
| CT | 29.91 30.7 | 33.21 33.1 | N |
| CT mean | 30.3 | 33.; | N |
| | 3.65E+0212.10E+02 | 3.84E+01 | N |
| | | 14.04E+01 | |
| Qmean | 2.B8E+02 | 3.94[+0.1 | N |

Result: after 3 days incubation, there was no difference between negative plasma dilution and drug dilution in CT value at 2 fold dilution. The CT value in negative plasma dilution at 10 fold dilution was 2

CT advanced than drug dilution.

In vitro anti-HBV efficacy test

Method and materials

1) Cell model: HepG2 cell infected with HBV virus, which is HepG2 2.2.15 cell

2) Cell viability is analyzed by MTT method

3) EIA test to detect the inhibition of HBsAg and HBeAg

4) Positive control drug: Lamivudine

5) RT-PCR detection of HBV-DNA

Procedure

1) Toxicity of drug to cell

HepG2 2.2.15 cells are seeded in 96-well plate. Fresh medium. With various concentration of drug is added 48 hour later. Cell viability is analyzed 9 days later by MTT method.

2) The inhibition of HBV virus

EiepG2 2.2.15 cells are seeded in 96-well plate. Fresh medium with various concentration of drug is added 48 hour later. The HBsAg and HBeAg are detected 5 days, 7 days, and 10 days later. RT-PCR detection of HBV-DNA

Results

| | |
|---|---|
| HBsAg | HBeAg |

| i\FOD | | Inhibition | | Inhibition |
|---|---|---|---|---|
| (!J · g/rnL) | OD | rate % | 00 | rate % |
| 10 | 0.611 | 47.6 | 1.020 | 17.6 |
| 5 | 0.695 | 40.4 | 1.059 | 14.5 |
| 2..5 | 0.7!5 | 33.5 | 1.115 | 10.0 |
| 1.2.5 | 0.897 | 23.1 | 1.165 | 5.9 |
| Negative control | 1.166 | I | 1.238 | I |

Quantification Test Results for HBV and HCV

| Sample Name | Quantification Test Results (IU/ml) |
|---|---|
| 105 HCV + AFOD--KH | 2.8E+04 |
| 105 HCV + AFCC-RAAS-2 | 8.1E+05 |
| 105 HCV + AFCC-RAAS--6 | <25.0 |
| 500 HBV + AFOD-KH | 8.18E+1 |
| 500 HBV + AFCC-RAAS-2 | <2.00E+1 |
| 500 HBV + AFCC-RAAS-6 | 5.04E+1 |
| 500 HBV + AFC:C-RAAS-8 | <2.00E+1 |
| 500 HBV + Negative Plasma | 4.41E+1 |

Note:
The detection limit for HBV quantification is 2.00E+11 U/mL

| | |
|---|---|
| 105 HCV + AFCC-RAi\S-8 | 2.4E+05 |
| 105 HCV + Negative Plasma | 2.11E+3 |

Note:
The detection limit for HBV quantification is 2.5 IU/ml.

FIG. 46

FIG. 47

FIG. 47a

FIG. 48

FIG. 49

FIG. 50

FIG. 50a FIG. 50b FIG. 51

FIG. 52.

In vitro studies of the KH mediums using to express the cultured cells in order to obtain a desired protein.

KH 101 Medium Alone KH1011 Medium alone FIG. 53.

KH101 medium alone—Nearly 20 million cells

FIG. 54

KH 101 Medium with AFCC product

AFC:C: alone—8,000 cell count

FIG. 55

AFCC with KH101 medium

FIG. 56

AFCC with KH101 medium after 5 days 4.5 million cell count

FIG. 57

KH 101 Medium with APOA1 product

APOAlalone—20,000 cell count

FIG. 58

APOA1 with KH101 Medium

FIG. 59

APOA1 with KH101 medium after 5 days 4 million cell count

FIG. 60

KH 101 Medium with AFOD Product

AFOD alone—10,000 cell count

FIG. 61

AFOD with KH101 medium

FIG. 62

AFOD with KH101 medium after 5 days—4.6 million cells

FIG. 63

KH 101 Medium with Factor VIII product

Factor VIII alone—5,400 cells

FIG. 64

Factor VIII with KH101 medium

FIG. 65

Factor VIII with KH101 medium after 5 days—3.4 million

FIG. 66

IN VIVO STUDIES

The study of APOAI protein in preventing atherosclerosis and related cardiovascular diseases

Study conducted h1: Fudan University, Zhang Jiang cmnpus

Department: School ofPhannacy, Fudan University

Original data kept in: School of Pharnlacy, Fudan University

The current study was designed to investigate the human serum APOAI protein in preventing the atherosclerosis. New Zealand rabbits were adopted in this animal study and divided into 5 groups. They were high dose, medium dose and low dose of treatment, positive and vehicle control. The treatment groups were given APOAJ via auricular vein once a week Vehicle controls received normal saline via auricular vein once a week. Positive controls were given Liptor daily by p.o. with a dose of 0.45 mg/kg body weight. The body weight of animal was determined every week and whole blood was drawn every three weeks. The study duration was 19 weeks. At the end of study, all animals were sacrificed. The important organs like liver, heart, kidney, aorta, and arteria carotis were observed in gross and pathological sections. Lipid content

was examined in liver and aorta. And liver index was also determined. Results showed that there was no significant change in body weight. The HDL-C was significantly high in ail treatment groups when compared with vehicle control. Although the liver index was lower in treatment group, but there's no statistical difference found. The area of atherosclerosis was significant less in medium group when compared with vehicle control. The pathological examination showed that there was no calcification found in either vehicle control or treatment group. However there was one animal with calcification in positive control group. The pathological change of aorta

was better in medium group when considering endothelium swelling, smooth muscle migrating and foam cell formation compared with vehicle control. But there is no significant improvement in low dose group. The cellular swelling and fat degeneration was better in the liver of medium than that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.

Purpose of the Experiments:

To investigate the human serum APOAI in in preventing atherosclerosis and related cardiovascular diseases and provide experimental basis for clinical application.

Methods and materials

1, Tested reagent

Product name: human Apoiipoprotein AI, injection Produced By: Shanghai RAAS Blood Products Co. Ltd. Lot number:

Size: 50 mg/mL

Appearance: colorless liquid

Positive control: Liptor

2. Animal

Strain: New Zealand white rabbit

Vendor: Shanghai JieSiJie Laboratory Animal Co., Ltd

Qualification number: Sex: male

Body weight: 1.8-LO kg

3 high fat diet recipe

1%) cholesterol+99 normal diet, provide by Shanghai SiLaiKe Laboratory Animal Center

4 Experimental Design

4.1 Model

Male New Zealand white rabbits were used in this study. The body weight was between 1.8-2.0 kg. The animals were quarantined fix 5-10 days With normal diet before study. Blood samples were taken 12 hour after fasting before study to determine the blood lipid parameters.

4.2 Group

Animals were randomly divided into 5 groups including vehicle control, high dose, medium dose, 1 mv dose and positive control group. Ten to 14 rabbits were in one group. Each rabbit was fed with 30 gram of high fat diet formed by 120 gram of normal diet with free access to water.

Housing condition: Ordinary Animal Lab with temperature of 24J-:2 OC and humidity of 55<%±10%.

4.3 Administration

First dose was given 1 week before high fat diet. The frequency of dosing was once a week Dose was 80, 40, 20 mg/kg body weight respectively. Drug was given by intravenous injection via auricular vein with the volume of 5 mL.

Liptor was given by intragastric administration

5 parameters tested:

5.1 body weight: body weight of each rabbit was determined once a Week.

5.2 blood lipid parameters: whole blood was drawn every three weeks. Animals were subject to 12 hour fast before taking blood. Resulted blood samples were kept still for 2 hours and then spin with 4,000 rpm for 10 min. The upper layer of serum was then separated and examined for total cholesterol (TC), total triglyceride (TG), low density lipoprotein cholesterin (LDL-C), and high density lipoprotein cholesterin (HDL-C). Test reagents were purchased from Shanghai

Rong Sheng Rio-pharmaceutical Co. Ltd.

5.3 Pathological examination

A: The atherosclerosis of aorta (plaque area lj)

B: Liver index

C: Aorta, liver, heart, arteria carotis, kidney

Results

1 The establishment of animal model

Animals were fed with high kd diet and treatment as described above. All blood lipid parameters significantly increased. There was no significant difference between vehicle control and

treatment groups (data shown below). After 12 weeks of high fat diet, 1 animal in vehicle control or treatment group was sacrificed respectively. The liver of animal in vehicle control showed cream white in color and there was no atherosclerosis observed in aorta. There was no abnormal change in the liver and aorta of animal in treatment group. After 16 weeks of high fat diet, 1 animal of vehicle control was sacrificed and found about 20% of plaque on the inner surface of aortic arch. Animal continued to be fed with high fat diet and treatment for 3 more weeks. After 19 weeks of high fat diet, all animals were sacrificed.

2 Animal procedures and tissue sampling

All animals were anesthetized by 20 of ethyl carbamate and then sacrificed with air injection. Abdomen cavity was opened. Whole blood was taken from heart. Heart was harvested along with 7 em of aorta. Then other organs like liver, kidney and arteria carotis were harvested. Connective tissue was stripped from resulted organs or tissues followed by washing in normal saline fix 3 times. Pictures were taken then.

Aorta was cut from aortic arch, opened longitudinally and taken picture. The aorta was dissected for 0.5 em from aortic arch, split longitudinally and then kept in cryo-preservation tube for later lipid analysis. One piece of this sample was fixed in fomlalin for further pathological analysis.

The weight of liver was determined immediately. Two pieces of specimen were cut from hepatic lobe. One was kept in cryo-preservation tube for lipid analysis and another one was fixed in formalin for further pathological analysis.

One piece of kidney sample was taken from renal pelvis and fixed in fomlalin for further pathological analysis.

Arteria carotis was dissected, cleaned and fixed in Formalin for further pathological examination.

The Formalin solution was replaced by fresh one about 4 hours and sent to pathological depmiment for pathological section.

3 Results

3.1 Change of body weight

The body weight of each animal was determined before high fat diet and once a week thereafter. The change of body weight in each group was shown in table 1.

The change of body weight in different groups

| Group | | | | |
|---|---|---|---|---|
| (animal | \VkO | ‘\Vk 19 | [ncrease | lncrease |
| number) | (kg) | (kg) | (kg) | (%) |
| Vehicle (n = 9) | 1.94 ± 0.231 | 3.23 ± 0.284 | 1.29 ± 0.361 | 66.5% |
| High dose | 1.68 ± 0.078 | 3.49 ± 0.221 | 1.81 ± 0.209 | 107.1%; |
| (n === 8) | | | | |
| Medium dose | 1.8 ± 0.22 | 2.99 ± 0.52 | 1.18 ± 0.286 | 65.5% |
| (n = 9) | | | | |
| Low dose | 2.Ll-AU74 | 3.19-.-i-:( ).278 | 1.09 .+:JL529 | 51.9% |
| (n === 12) | | | | |

3.2 Plasma lipid parameters

Animals were fast for 12 hours before taking blood samples via auricular vein. Resulted blood samples were kept sti ii f;x 2 hours. The upper layer of serum was then separated and examined ± or total cholesterol (TC), total triglyceride (TG), 1 mv density lipoprotein cholesterin (LDL-C), and high density lipoprotein cholesterin (HDL-C). Test reagents were purchased from Shanghai Rong Sheng Bio-pharmaceutical Co. Ltd.

TABLE 2
Change of total triglyceride (TG)

| Group | | | | |
|---|---|---|---|---|
| (animal | ‘\VkO | ‘\Vk 19 | Increase | Increase |
| number) | (mmol/L) | (mmol/L) | (mmol/L) | (%) |
| Vehicle | 0.823J:0.294 | 1.864-.-H).871 | 1.04H.-0.933 | 126.5 |
| (n = 9) | | | | |
| Medium | 0.656 ± 0.19 j | 2. j 44 ± 1.043 | 1.488 ± 0.988 | 226.8% |
| dose (n = 9) | | | | |
| l,ow dose | 0.786 ± 0.229 | 1.267 ± 0.772 | 0.482 ± 0.839 | 61.3lj |
| (n = 12) | | | | |

TABLE 3
Change of total cholesterol (TC)

| Group | \VkO | ‘\Vk 19 | Increase | Increase |
|---|---|---|---|---|
| (anim.al mnnber) | (mmol/L) | (mmol/1,) | (m.moliL) | (%) |
| Control(n = 9) | 1.15 ± 0.23 | 8.049 ± 2.99 | 6.896 ± 3.03 | 598.3% |
| High dose (n === 8) | 1.59 .t-J}.48 | 12.49 -t-2.81 | 10.90J:2.66 | 685.5% |
| Mediumdose(n = 9) | 1.77 ± 0.783 | 10.28 ± 5.82 | 8.505 ± 5.37 | 453.0% |
| l,ow dose (n = l2) | 1.06.-i-:0.27 | 9.07-.+:4.92 | 8.0Lt-A.87 | 755.6% |

TABLE 4
Change of high density lipoprotein cholesterin (HDL-C)

| ⁻Group | \VkO | \Vk19 | Increase | Increase | |
|---|---|---|---|---|---|
| (animal Iunnber} | (m.moliq | {m.moliq | (m.moliq | C %} | Sig |
| Control(n = 9) | 0.94 ± 0.262 | 3.527 ± 2.007 | 2.588 ± 1.918 | 275.3%) | |
| High dose (n = 8) | 1J 83 + 0.149 | 4.993 · −+ :2.018 | 3.8H2.025 | 322.1 − ‘0 | 0.( )35* |
| Mediumdose(n = 9) | 0.67 ± 0.207 | 4.343 ± 2.439 | 3.674 + 2.413 | 548.4% | o.ol |
| Low dose (n = 12) | 0.705 ± 0.246 | 3.744 ± 2.14 | 3.04 ± 2.019 | 431.2′% | 0.028* |

p < 0.05

TABLE 5
Change ofligh density lipoprotein cholesterin (LDL-C)

| Group | \VkO | Wk 19 | Increase | Increase |
|---|---|---|---|---|
| (anim.al mnnber) | (rnmol/I.) | (rnrnol/L) | (mm.ol/L) | (%) |
| Control(n = 9) | 0.872 ± 0.386 | 5.826 + 2.909 | 4.954 ± 2.953 | 568.1% |
| High dose (n = 8) | 0.92 ± 0.324 | 14.1 ± 4.188 | 13.18 + 4.053 | 1432.6% |
| M ediumdose(n ==== 9) | j .06 ± 0.298 | 6.357 ± 4.475 | 5.297 + 4.373 | 499.7%; |
| Low dose (n = 12) | 0.826 ± 0.279 | 7.298 + 4.60 | 6.472 ± 4.468 | 783.5 · |

TABLE 6
Liver index

| Group | Body weight | Liver weight | Uver index | |
|---|---|---|---|---|
| (animal number) | (kg) | (g) | (%) | Sig |
| Control(n === 9) | 3.083:1:.0.279 | 123.08 −+ .−22.31 | 3.984:1:.0.579 | |
| High dose (n = 8) | 3.565 ± 0.205 | 151.69 ± 18.49 | 4.257 ± 0.482 | 0.26 |
| Mediumdose(n = 9) | 3.009−.−i−:0.554 | 112.006− −+ .−25.79 | 3.708−.−i−:( ).391 | 0.267 |
| Low dose (n = 12) | 3.3 ± 0.329 | 128.096 ± 20.43 | 3.886 ± 0.489 | 0.571 |

3.3 Plaque area of aorta

The aorta was dissected and opened for 7.5 em from aortic arch longitudinally. Pictures were taken and atherosclerosis changing was analyzed. The area of atherosclerosis was graded by clinical standard according to its area to whole area of dissected aorta, by which grade I was less than 25 ?-), grade H was between 25% to 50%, grade HI was between 50% to 75% and Grade IV was greater than 75%.

TABLE 7
atherosclerosis change in vehicle control group

| Animal number | Plaque area/amia area | Grade |
|---|---|---|
| 5 | 8.62 | I |
| 6 | 16.67 | I |
| . . . , | | n |
| 9 | 39.47 | II |
| 11 | 1.67 | |
| 12 | 10 | I |
| 17 | 92.86 | IV |
| 18 | 70.91 | n |
| 19 | 25.17 | ll |

Grade I: 4 animals; Grade II: 4 animals; Grade HI: 0 animal; Grade IV: 1 anirnai.

TABLE 8
atherosclerosis change in low dose group

| Animal number | Plaque area/aorta area | Grade |
|---|---|---|
| 31 | 10 | I |
| 32 | 26 | II |
| 36 | 1.92 | I |
| 37 | 76.79 | III |
| 38 | 11.11 | I |
| 39 | 2.88 | I |
| 40 | 6.67 | I |
| 41 | 2 | I |
| 42 | 92 | IV |
| 43 | 6.67 | I |
| 44 | 0.18 | I |
| 48 | 23.36 | I |

Grade I: 9 animals; Grade II: 1 animal; Grade HI: 0 animal; Grade IV: 2 animals.
Statistical analysis of low dose group: Mann-Whitney test
I I I
I
Level sum in Vehicle controL 112.8
Level sum in lovv dose group: 116.5
To.os″′71 T > To.os no statistical difference

TABLE 9
atherosclerosis change in medium dose group

| Animal number | Plaque area/a01ia area | Grade |
|---|---|---|
| 21 | 36.53 | II |
| | 1.69 | |
| 23 | 18.75 | I |
| 25 | 19.17 | I |
| | 11.67 | I |
| 28 | 1.82 | I |
| 29 | 61.67 | II |
| 30 | 1.6 | I |

Grade I: 6 animals; Grade II: 2 anirna!s; Grade III: 0 animal; Grade IV: 0 animaL
tatistical analysis oflovv dose group: Mann-Whitney test
Grade 0 I I
Level 2 I I
Level sum in Vehicle control: 112.8
Level sum in low dose group: 46
To.os = 5 1 T < To.o.s statistical difference

TABLE 10
atherosclerosis change in high dose group

| Animal number | Plaque area/a01ia area | Grade |
|---|---|---|
| 50 | 62.5 | II |
| 51 | 100 | IV |
| 52 | 56.88 | II |
| 53 | 40.13 | II |
| 54 | 100 | IV |
| 55 | 27.19 | II |
| 60 | 68.03 | II |
| 62 | 95.00 | IV |

Grade I: 0 animal; Grade II: 5 animals; Grade III: 0 animal; Grade IV: 3 animals.

3A Pathological examination

3A.1 A01ia

Vehicle control

| | | Plaque | | | Smooth | |
|---|---|---|---|---|---|---|
| Animal | | (section) | | Endothelium | muscle | |
| number | Plaque (gross) | b | calcification | swellino | migrating | Foam cell |
| 5 | + | + | − | − | −+− | + |
| 6 | + | − | − | − | − | − |
| 7 | ++ | + | − | | − | + |

| | |
|---|---|
| 9 | ++ I + |
| 1l | − I − |

−− −=−−−−−−−−−−−−−−−−−−−−−−.−−− =−−−−−−−−−−−−−−−−−−−−−−−
−−−−−−−−−−−−−−−−−−−−−−−−−−−−−−t −−−−−−−−−−−−−−−−−−−−−−−−−−−
−−−−−−−1−−− −−−−−−−−−−−−−−−−−−−−−−−−−−−−−−−−− −−−−−−−−−−−−−−−−−−−−−−−−−−−−−−− −−−−−−−−−−−−−−−−−−−−−−−−−−−−−−−

| | |
|---|---|
| 13 | : −− : − : − : − − − |

−−−−−−−−−−−−−−−−−−−−−−−−−−−−−−1−−−−−−−−−−−−−−−−−−−−−−−−−−−−−−−−−−−−−−−−−−−−−−−−−−−−−−−−−−−−−t−−−−−−−−−−−−−−−−−−
−−−−−−−−−−−−1−−−−−−−−−−−−−−−−−−−−−−−−−−−−−−−−−−−−−−−−−−−−−−−−−−−−−−−−−−−−−−−−−−− −−−−−−−−−−−−−−−−−−−−−−−−−−−−−−

| | |
|---|---|
| 17 | !+++ ++ !− i++ + ++ |

−−−−−−−−−−−−−−−−−−−−−−−−−−−−−−1−−−−−−−−−−−−−−−−−−−−−−−−−−−−−−−−−−−−−−−−−−−−−−−−−−−−−−−−t−−−−−−−−−−−−−−−−−−−−−−
−−−−−−−−−−−−−1−−−−−−−−−−−−−−−−−−−−−−−−−−−−−−−−−−−−−−−−−−−−−−−−−−−−−−−−−−−−−− −−−−−−−−−−−−−−−−−−−−−−−−−−−−−−

| | |
|---|---|
| 18 | i +++ + i − i++ + |

−−−−−−−−−−−−−−−−−−−−−−−−−−−−−−−1−−−−−−−−−−−−−−−−−−−−−−−−−−−−−−−−−−−−−−− −−−−−−−−−−−−−−−−−−−−+−−−−−−−−−−−−−−−−
−−−−−−−−−−−−−−−1−−−−−−−−−−−−−−−−−−−−−−−−−−−−−−−− −−−−−−−−−−−−−−−−−−−−−−−−−−−−−− −−−−−−−−−−−−−−−−−−−−−−−−−−−−−−

| | |
|---|---|
| 19 | i ++ + i − i + |

i i i
Medium dose group

| 21 | ++ + |
|---|---|
| 22 | |
| −:− | + |
| + | |
| 25 | + |
| 27 | |
| 28 | |

| | |
|---|---|
| 29 | +++ : T |
| 30 | i − ! − i − |
| | 1 1 1 −−−−−−−−−−−−−−−−−−−−−−−−−−−−−−−−−−−−−−−−−−−−−−−−−−−−−−−−−−−−− |

Lmv dose group

| 32 | ++ +++ +++ + +++ |
|---|---|
| 37 | ++ |
| 38 | |
| 39 | |
| 40 | |
| 41 | |

The pathological change was better in medium group when considering endothelium swelling, smooth muscle migrating and foam cell formation compared with vehicle control. But there is no significant improvement in low dose group

3.4.2 Liver gross and pathological examination

| | Observation (color, texture and size) Sv,.relling Fatty change |
|---|---|
| Anima# | Vehicle control |
| 5 | dark red, white m some area, soft, ++ |
| | -+- |
| | --- |
| | left>right |
| | Pink, soft, left>right |
| | +-- |
| | ] ! |
| 9 | pink,, less soft, + ! |
| 11 | Pink, smooth, soft ++ + ! |
| | i |
| 12 | pink, rough |
| | +++ + |
| 13 | dark red, some area shovved pink, + |
| | smooth, soft |
| 17 | Pink, partial rough, less soft + |
| 18 | Partial pink, smooth, soft |
| 19 | Partial pink, smooth, soft |

Medium dose group

| | |
|---|---|
| 21 | dark red, partial pink, soft, less smooth + + |
| 22 | ++ |
| 23 | dark red smooth, soft, left>right |
| 25 | dark red, partial pink, soft, smooth |
| 29 | dark red, soft, smooth |
| 30 | dark red, soft, smooth |

Low dose group

| | |
|---|---|
| 31 | Partial pink, soft, less smooth ++ |
| | Pink, soft, less smooth + -+- |
| 36 | Partial ye!lo\v, rough, less soft |
| 37 | Partial white, less soft, smooth |
| 38 | |
| 39 | Pink-white color, rough, less smooth |
| | +++ + |
| | ++ |
| 40 | Pink at Hepatic portal, soft, less smooth + |
| 41 | dark red, soft, smooth ! |
| 42 | Partial pink, soft, smooth + ! |
| 43 | dark red, soft, smooth |
| 44 | dark red, soft, smooth + |
| 48 | dark red, soft, smooth |

High dose group

| | |
|---|---|
| 50 | Partial yellow, rough surface, less soft ++ ++ |
| 51 | Yeilmv, rough surface, less soft |
| | ++ ++ |
| 52 | dark red, pmiial pink, rough surface,soft |
| 53 | Pink, rough surface, less soft +++ |
| 54 | Pink, rough surface, soft ++ |
| 55 | dark red, pmiial pink, rough surface, soft +++ |
| 60 | Partial yellow, rough surface, less soft + |
| 62 | dark red, pmiial pink, rough surface, soft ++ |

Positive control group

| | |
|---|---|
| 65 | Yellow, rough surface, less soft ++ |
| 66 | Yellow-white color, rough surface, less soft +++ |
| 68 | Pink-v,.rhite color at hepatic portal, dark red - at outskirt, rough texture, les soft |
| | --+2---r:v: -il --- - -i - ---;t---i p ti- ---i -rt 1:--- hit- --- t-- --++_+ --- |
| I | outskirt, rough texture, less soft I |
| | i ! |
| +3 | !Yell ow, rough texture, less soft +++ |

The cellular swelling and fat degeneration was better in the liver of medium than that of vehicle control. Although the cellular swelling was same in low dose group and vehicle control but the fat degeneration was better in liver of low dose group than that of vehicle control.

3.4.3 Hemi, Arteria carotis and kidney

| Animal | Heart/Coronary Arteria carotis kidney |
|---|---|
| number | Lipid plaque Lipid plaque Perirenal adipose I Pathological |
| ----------- | infiltration infiltration capsule I change |
| ----------- | ---------------------------------------------------------------------- |
| -- | ----------------------------------------------1---------------------- |
| 5 | --- |
| | Full, thick |
| 6 | Full, thin |
| 7 | Full, relatively - thick |
| 9 | Full, relatively - thick |
| 11 | Full, thin |
| 12 | Full, relatively - thick |
| 13 | Full, a little thick - |
| 17 | Full, a little thick - |
| 18 | |
| 19 | |

Medium dose group
Full, a little thick -Full, relatively - thick

| | |
|---|---|
| 21 | Full, thin |
| 22 | |
| 23 | Spots, thin |
| 25 | Full, very thin |
| 27 | Full, very thin |
| 29 | |
| 30 | |

Low dose group
Full, very thin
Full, very thin

| | |
|---|---|
| 32 | Full, very thin!- |

---------------------------------------------------------------------------
------------------------------------------------------L-----------------------

| | |
|---|---|
| 36 | Full, very thin |
| 37 | Full, thin |
| 38 | Full a little thick |
| 39 | Full a little thick |
| 40 | FulL relatively - thick |

----------------------------------------------------------------------------
---------------------------------------------------------+--------------------

| | |
|---|---|
| 41 | Full, a little thick - |
| 42 | |
| 43 | |
| 44 | |

High dose group
Full, relatively - thick
Full, very thin

| | |
|---|---|
| 50 | Full thick |
| 51 | Full |

thick
relatively -
relatively -
------------------------------------------------------------------------- ------
---------------------------------------------------!---------------------------

| | |
|---|---|
| 52 | Full relatively - |
| thin | |
| 53 | |
| 54 | |
| 55 | |
| 60 | |
| 62 | |

Positive control group

| | | |
|---|---|---|
| Full, | relatively | - thin |
| Full, | relatively | - thick |
| Full, | relatively | - thin |
| Full, | relatively | - thin |
| Full, | relatively | - thin |

| | |
|---|---|
| 65 | Less full, thin |
| 66 | Full, thin |
| 68 | Full, thin |
| +2 | Full, thin |
| +3 | Less full, thin |

There was no pathological change found in heart and kidney either in vehicle control or treatment groups. There was no atherosclerosis change found in Arteria carotis.

3.4.3 Lipid content in tissues

1) Lipid content in liver

| Con- | | | | |
|---|---|---|---|---|
| trol | | | | |
| Mid- | Lmv dose | | | |
| dle | High | | | |
| TC | 3.056 ± 0.775 | 2.95 ± 0.809 | 2,214 ± 0.515 | 2.841 ± 0.298 |
| TG | | | | |
| HDL- | 1.817 ± 0.446 | 1.369 ± 0.251 | 1.081 ± 0.31 | 1.3 ± 0.171 |
| C | 0.712 ± 0.244 | 0.803 ± 0.236 | 0.815 ± 0.249 | 0.825 ± 0.129 |
| LDL- | 2.035 ± 0.328 | [857 ± 0.559 | 1.407 ± 0.418 | 2.302 ± 0.054 |
| C | | | | |

| | Lovv dose | Medium | High |
|---|---|---|---|
| TC | 0.775 | 0.(22 | 0.564 |
| TG | 0.022 | O,,Oi t | 0.009 |
| HDL-C | 0.81 | 0.74 | 0.684 |
| LDL-C | 0.436 | OJ)] 1 | 0.989 |

Statistics analysis oflipid content in liver

The lipid content in liver showed that TG in low and high dose group was significantly lower than that in vehicle control. The TC, TG and LDL-C in medium group were significantly lower than those in vehicle control.

2) Lipid content in amia

| | Control | Lmvdose | Middle | High |
|---|---|---|---|---|
| TC TG | 0.331 ± | 0.28 ± | 0.332 ± | 0.29 ± |
| | 0.097 | 0.047 | 0J35 | 0.098 |
| ElDL-C | 0.406 ± | 0.337 ± | 0.388 ± | 0.402 ± |
| | 0.078 | 0.055 | 0.124 | 0.101 |
| LDL-C | 0.065 ± | 0.092 ± | 0.128 ± | 0.111 ± |
| | 0.032 | 0.066 | 0.064 | 0.057 |
| | 0.323 ± | 0.254 ± | 0.307 ± | 0.318 ± |
| | 0.116 | 0.078 | 0.043 | 0.05 |

Statistics analysis of lipid content in aorta

| | Lovv dose | Medium | High |
|---|---|---|---|
| TC | 0.387 | 0.8′79 | 0.483 |
| TG | 0.341 | 0.80 | 0.952 |
| HDL-C | 0.416 | 0.065 | 0.171 |
| LDL-C | 0.138 | 0.73 | 0.9l2 |

The lipid content in aorta was lower in treatment groups than that in vehicle control but there was no statistical significance.

Summary:

This study was designed to investigate the prevention efficacy of APOA1 in atherosclerosis. The test article was given along with high fat diet which caused no significant decrease in blood lipid parameters. However the treatment significantly increased the HDL-C level in all treated groups. There was no dose escalation effect found in three treatment groups upon anatomic, pathological and biochemistry examination. It has been showed that the atherosclerosis in medium dose group was significantly less than that in vehicle control. The pathological change was better in medium group when considering endothelium swelling, smooth muscle migrating and foam cell formation in aorta compared with vehicle control. But there is no significant improvement in low dose group. The cellular swelling and fat degeneration was better in the liver of medium than

that of vehicle control. Although the cellular swelling was same in low dose group and vehicle control, but the fat degeneration was better in liver of low dose group than that of vehicle control. The lipid content in aorta was lower in treatment groups than that in vehicle control but there was no statistical significance. The lipid content in liver showed that TG in low and high dose group was significantly lower than that in vehicle control. The TC, TG and LDL-C in medium group were significantly lower than those in vehicle control.

FIG. 67

FIG. 68

FIG. 69

From vehicle and treated two rabbits, sacrificed and operated to determine the fat build up during the first 8 weeks of the study.

### APPENDIX 1: PICTURES OF AMIA

Vehicle control

Low dose group FIG. 70

[symbol]

Medium dose group

FIG. 71

[symbol]

FIG. 72

High dose group

FIG. 73

[symbol]

Positive control (Lipitor)

FIG. 74

The lipid profile results and quantification of atherosclerosis pla(JUe in 18 i\poE tnice for 4
‘veeks stduy ®
27-1\tlarch-2012
11-,Jan-2012 Owk 7-Feb-2012 13--Fet>-2012 4 wks 13_Mar-ZOiZ 9 wks 16-Mar-2012
9 wks [symbol] [symbol] [symbol] U t<J pr4f1le
t_; w:1 r.;•nitlt”’ L<piC prctl:e
m•osureme:1t HFD :‘,”’-et s,. ;, :;· nt Groupig and rr.eaouro.m nt All18 mie were
sacrificed
befrn-e HFD :. .. ... Nle14 doso(5 wk5) ren m n and Aortas 1;′\tere dissected
starting treatment
18 male Apo E (-/-)were fed with HFD/ High cholesterol diet starting on .hn.11, 2012 [symbol]
18 Apo E{-/-) mice were assigned to 4 groups based on the BW,TC, HDL level after fed with
HFD
for 4 weeks and all mice were treated with test articles starting nn Fdd 3, 2012.
Vehicle
ApoA1 0.2 ml iv/ip n=5
AFOD 0.2 ml iv/ip n=4
AFCC 0.2 ml iv/ip n=4
“Collected 300 ul of blood for lipid profile measurement on 13-Mar-2012 after 14dose(S wks)
treatment. AH the mice were sacrificed on March 16 and all AORTA were dissected for
atherosclerotic plaque analysis by oil red staining later.
Body weight in 18 ApoE mice

FIG. 75

. . . :-. t ooks Hk 2: thn$ ni: bods d -dn't dL:sturt3 th:3 ncr 3.: 3E3 Gf bt>dy ′N•3j lht ;n tho: 3=mk:3 aftr 6 . . . l'E3 k

tr tm •n

Blood plasma lipid profile at three time points in 18 Apo E(−/−} mice

FIG. 76

FIG. 77

FIG. 78

FIG. 79
18 Apo E(−/−) mice at 8 weeks old were fed with HFD/High Cholesterol diet for 4 weeks. Then were treated with AFCC, APOA1 and AFOD for 5 weeks. It looks like three antibodies didn't improve the lipid profile in those mice after 5 weeks treatment.
Three time points: 0 week: Before HFD; 4 week: Fed with HFD for 4 week; 8 week; After 4 weeks treatment

Illustration of AORTA

Sites of predilection for lesion development are indicated in black: (1) aortic root, at the base of the valves;

(2) lesser curvature of the aortic arch;

(3) principal branches of the thoracic aorta; (4) carotid artery;

(5) principal branches of the abdominal aorta; (6) aortic bifurcation;

(7) iliac artery; and

(8) pulmonary arteries.

Quote from Y Nakashirna, 1994

FIG. 80

Oil Red staining procedure:
Sacrificed the mice and heart, aorta, and arteries were dissected under the dissecting microscope.

Briefly wash with PBS and fixed in 4% paraformaldehyde (PFA) overnight at 4° C. Rinse with 60% isopropanol

Stain with freshly prepared Oil Red 0 working solution 10 mins.

Oil red 0 stock stain: 0.5% powder in isopropanol

Working solution: dilute with distilled water (3:2) and filter with membrane
Rinse with 60% isopropanollO second.

Dispel the adherent bit fat outside of the aorta under the dissecting microscope.
Cut the vascular wall softly and keep the integrated arteries using the microscissors.

Unfold the vascular inner wall with the cover and slides glass and fix it by water sealing tablet.

Image analysis procedure:
The unfolded vascular inner wall “Were scanned with Aperio ScanScope system and the area of atherosclerotic plaque was measured by Image-Proplus software after oil red 0 staining as follow picture shown.

FIG. 81a.

Photos:

FIG. 81b.

Results:

We measured the sum lesion areas and mean density using ipp software and calculated atherosclerotic percent.

Area percent (%)” Sum area of atherosclerotic plaque (mm2) I whole area of vascular inner wall

(mm2)

FIG. 81c FIG. 81d FIG. 81e

Summary:
The atherosclerotic plaques/lesions were obviously labeled in the luminal surface area of the aorta compared with the control. This results is consistent with the published literatures. The atherosclerotic animal model was established in Apo E(−/−) mice fed with the high fat diet for 9 weeks.
ApoA1 showed a trend on reducing the atherosclerotic plaques/lesions compared to the vehicle group after 14 dosing.

### REFERENCE

Y Nakashima et al. A poE-deficient mice develop lesions of all phases of atherosclerosis throughout the alierial tree. Arteriosclerosis and Thrombosis Vol 1 4, No 1 Jan. 1994

Initial Report of Efficacy Study on

RAAS AFOD RAAS 1 (APOA1) in ApoE mice for 8 weeks

Study Title: Efficacy study of 4FOD RAAS 1 (AP( ) 41) on atherosclerosis model in ApoE nlice

Study Number: CPB-Pll-2504-RAAS

Date: Jun. 29, 2012

### 1. Abbreviations and Definitions

| | |
|---|---|
| kg | kilogram |
| g | gram |
| Mg | milligram |
| ng | Nanogram |
| ml | Milliliter |
| !JL | microliter |
| h | hours |
| min | minutes |
| Cpd | Compound |
| BW | Body Weight |
| BG | Blood Glucose |
| FBG | Fasting Blood Glucose |
| DOB | Date of Birth |
| TC | Total Cholesterol |
| TG | Triglyceride |
| LDL | Low Density Lipoprotein |
| HDL | High Density Lipoprotein |
| FBW | Fasting Blood Glucose |
| so | Standard Deviation |
| SE | Standard error |
| i.p | Intraperitoneal injection |
| PFA | paraformaldehyde |

### 2. Introduction

The study described in this report evaluated in vivo efficacy of RAAS antibody

APOA1 in atherosclerotic model.

### 3. Purpose

To evaluate the efficacy effect of RAAS antibody APOA1 on plasma lipid profile, lesion plaque of inner aorta and related parameters in atheroslerotic model.

### 4. Materials

4.1. Test article: RAAS APOA I; Atorvastatin (reference compound)

4.2. Animal: ApoE knock out (ko) mouse

Sex: male

Strain: C57BLKS

Vender: Beijing Vitol River

Age: 8 weeks (arrived on 23 Dec. 2011) Number: 60

4.3. Upid profile test: Shanghai DaAn Medical laboratory, Roche Modular automatic biochemistry analyzer

4.4. Heparin Sodium Salt: TCI_, H0393

4.5. Capillary: 80 mm, 0.9-1.1 mm

4.6. Ophthalmic Tweezers and scissors: 66 vision-Tech Co,. LTD, Suzhou, China. Cat#53324A, 54264TM

4.7. High Fat diet: TestDiet, Cat#58v8(35% kcal fat 1% chol)

4.8. Glycerol Jelly Mounting Medium: Beyotime, Cat# C0187.

4.9. Glucose test strips: ACCU-CHEK Performa: ROCHE (Lot#470396)

4.10. Image analyse: Aperio ScanScope system; Image-Proplus 6.0 software; Aperio image scope version 11.0.2.725 software.

4.11. Aorta staining: Oil Red 0 (Alfa Aesar) Isopropanol (Lab partner)

### 5. Experiment Method

5.1. Grouping mice:

10 ApoE ko mice were fed with regular chow diet and used as negative control group. 50 ApoE ko mice were fed with high fat diet (35% kcal fat, 1% cholesterol) for 8 weeks, and then the plasma samples were collected for lipid profile measurement before the treatment. 50 ApoE ko mice were assigned into 5 groups based on the fasting overnight plasma TC and HDL level. The group information is shown in the table below.

TABLE 1
lnformatlon of groups

| Group | ApoE ko mice | Diet | Solution | Cone″ Of CPD | Formulation |
|---|---|---|---|---|---|
| r−−−N−egative−− | −−−−−−−−−−−−;:; − | −−−−−−N−;;;:;r;− | −−−−−−−−−−−−−−− | −−−−−−−−−−−−−−−−− | −−−−−−−−−−−−− |
| c;;;:;!;:c;l | 1 − o | aT−aTet | −−−−−−−−−−−−−−− | −−−−−−−−−−−−−−−−− | −−−−−−−−−−−−− |
| [:−− − −− − − : − −−−−− | −−−−−−−−−−− − − | | −−−−−− − −: −− −:− | −−−−−−−−−−−−−−−−− | −−−−−− |
| −−−−−−−−−−−−−−−−−−−−−−− | − −−−−−−−−−−−− | −−−−−− − − −− − − | − −−−−− | −−−−−−−−−−−−−−−−− | −−−−−−−−−−−−− |
| 1 High Dose: | n = 10 | − − − − −−−−− | −−−−−−−−−−−−−−− | − | −−−−−−−−−−−−− |
| 0.1 ml Lp q.o d | −−−−−−−−−−−−−−− | High fat diet | −−−−−−−−−−−−−−− | 5% Protein | −−−−−−− |
| −−−−−−−−−−−−−−−−−−−−−−−−− | −−−−−−−−−−−−−−− | −−−−−−−−−−−−−−− | | −−−−−−−−−−−−−−−−− | −−−−−−−−−−−−− |
| −−−−−−−−−−−−−−−−−−−−−−−−− | −−−−−− | −−−−−−−−−−−−−−− | | −−−−−−−−−−−−−−−−− | −−−−−−−−−−−−− |
| −−−−−−−−−−−−−−−−−−− | n = 10 | −−−−−− | | − | −−−−−− |
| ! Mid Dose: | | High fat diet | | 5% Protein | |
| 0.075 ml i.p q.o | | | | | |
| 1 d | | | | | |
| Low Dose: 0.0.05 rnl | n = 10 | High fat diet | | 5% Protein | |
| i.p q.o | | | | | |
| d | | | | | |
| Positive Control | n = 10 | High fat diet | 0.5% Gv1C | 2 rng/ml | 20 mg + 10 ml |
| (Atorvastatin) | | | | | 0.5% CMC |
| 20 mg/kg | | | | | |

5.2. Study timeline:

23 Dec. 2011: 60 ApoE mice arrived at chempartner and were housed in the animal facility in the building#3 for the acclimation.

6 Jan. 2012: Measured the body weight for each mouse” 50 mice were fed with high fat diet and 10 mice were fed with normal chow diet”

2 Mar. 2012: Ail mice were fasted over night and plasma samples (about

300 ul whole blood) were collected for lipid profile measurement before treatment with RAAS antibody,

19 Mar. 2012 to 6 Apr. 2012: Group the mice based on the TC and HDL level and start the treatment with 3 doses of antibody APOA1 by i.p daily on the weekday (The first dose was administered by iv injection

through the tail vein. The reference compound atorvastatin was administered by oral dosing every day.

7 Apr. 2012 to 12 Apr. 2012: Stop dosing for 5 days. After 15 doses treatment with the antibody, several mice died in the treatment groups. The client asked for stopping treatment for a while.

13 Apr. 2012-14 May 2012: The treatment with antibody APOA1 was changed to i.p injection every two days (Monday, Wednesday, and Friday) per client's instruction.

17 Apr. 2012: All mice were fasted over night and plasma sample for each mouse (about 300 ul whole blood) was collected for lipid profile measurement after 4 weeks treatment.

14 May 2012: Ali mice were fasted over night and plasma sample for each mouse (about 300 ul whole blood) was collected for lipid profile measurement after 8 weeks treatment. Blood glucose was also measured for each mouse.

17 May 2012: The study was terminated after 8 weeks treatment. Measure BW, sacrificed each mouse. dissected the aorta, heart, liver and kidney and fixed them in 4% PFA.

5.3. Route of compound administration:

Antibody products were administrated by intraperitoneal injection every two days (Monday, Wednesday, and Friday). and the positive compound was administered by p.o every day.

5.4. Body weight and blood glucose measurement: The body weight was weighed weekly during the period of treatment. The fasting overnight blood glucose was measured at the end of study by Roche glucometer.

5.5 24 h food intake measurement: 24 hours food intake for each cage was measured weekly

5.6. Plasma lipid profile measurement: About 300 ul of blood sample was collected from the orbital vein for each mouse and centrifuged at 7000 rpm for 5 min at 4° C. and the plasma lipid profile was measured by Roche Modular automatic biochemistry analyzer in DaAn Medical Laboratory

5.7. Study taken down:

After RAAS antibody products treatment for 8 weeks, all mice were sacrificed. Measured body weight and collected blood sample for each mouse. Weighed liver weight and saved a tiny piece of liver into 4% paraformaldehyde (PFA) fixation solution for further analysis. At same time, take the photos with heart, lung, aortas and two kidneys.

5.8. Oil Red staining procedure:

1. Sacrificed the mice and dissected the heart, aorta, and arteries under dissecting microscope.

2. Briefly wash with PBS and fixed in 4% paraformaldehyde (PFA) overnight at 40 C.

3. Rinse with 60% isopropanol

4. Stain with freshly prepared Oil Red 0 working solution 10 min.

1). Oil red 0 stock stain: 0.5% powder in isopropanol

2). Working solution: dilute with distilled water (3:2) and filter with membrane (0.22 um)

5. Rinse with 60% isopropanol 10 second.

6. Dispel the adherent bit fat outside of the aorta under the dissecting microscope.

7. Cut the vascular wall gently and keep the integrated arteries using the micro scissors.

8. Unfold the vascular inner wall with the cover slides and fix it by water sealing tablet.

5.9. Image scanning and analysis:

Scanning the glasses slides with the Aperio ScanScope system and analyze with the image proplus software to measure the area of atherosclerotic plaque session. The results were expressed as the percentage of the total aortic surface area covered by lesions. The operation procedure of software was briefly described as follow: Converted the sys version photos into JPG version, then calibrated it and subsequently selected the red regions and then calculate the total area automatically by image proplus software.

5.10. Clinic observation:

Atorvastatin significantly reduced the body weight after 5 weeks treatment. APOA1 showed a trend on reducing body weight but didn't reach statistic difference compared to the vehicle group. Total 5 mice from different groups died during the 5 months study period due to kidney infection or Lv injection or the accident while performing blood collection. The information of dead animals was

shown in the table below and the more detail information about dead mice was listed on the sheet of clinic observation of raw data file.

TABLE 2
The information of dead and wounded mice

| Group | Dead | Reason | Wounded | Reason |
|---|---|---|---|---|
| Negative control | 1 | No reason disappeared | 0 | fighting |
| Vehicle Saline | 1 | Died and the unclear | 2 | each |
| | | reason | | other |
| APOA 1high dose | 2 | Kidney infection & | 1 | |
| | | i.v injection | | |
| APOA 1mid dose | 1 | Blood collection | 1 | |

### 6. Data Analysis

The results were expressed as the Mean±SEM and statistically evaluated by student's t-test. Differences were considered statistically significant if the P value was <0.05 or <0.01.

### 7. Results

7.1. Effect of APOA1 on Body Weight

FIG. 82. Body Weight

The body weight in Apo E knockout mice fed with HFD significantly increased after 6 weeks treatment compared with the mice in negative control group that were fed with normal diet. Atorvastatin significantly reduced the body weight after 5 weeks treatment. APOA1 showed a trend on reducing body weight but didn't reach statistic difference compared to the vehicle group.

7.2. Effect of 24 food intake.

FIG. 83. 24 h food intake

As shown in FIG. 21 mice in the negative control group eat a little bit more than the mice fed with HFD but no statistic difference.

7.3. Effect of HFD on lipid profile in ApoE ko mice

FIG. 84. Compare the lipid profile of ApoE mice fed with common diet and high fat diet. The lipid profile was measured in Apo E ko mice fed with high fat diet for 8 weeks. As shown above, plasma TC, TG, LDL as well as HDL in Apo E ko mice fed with high fat/high cholesterol for 8 weeks were significantly increased compared to Apo E KO mice fed with normal chow diet.

7.4. Effect of RAAS antibody on total cholesterol (TC)

FIG. 85, Plasma TC

FIG. 86. Net change of plasma TC

As shown in the figure above, positive control atorvastatin and low dose of APOA1 can significantly lower total cholesterol level after 8 week treatment in ApoE ko mice after 8 week treatment.

7.5. The effect of RAAS antibody on Triglyceride (TG}

FIG. 87. Plasma TG

As shown in figure above, positive control atorvastatin and RAAS antibody had no effect on plasma TG level in Apo E ko mice fed with HFD after 8 weeks treatment.

7.6. The effect of RAAS antibody on High Density lipoprotein (HDl}

FIG. 88. Plasma HDL

As shown in FIG. 6, positive control atorvastatin can significantly lower high density lipoprotein in Apo E ko mice fed with HFD after 8 week treatment and RAAS antibody at low dose significantly decrease the HDL level in ApoE ko mice after 4 weeks treatment.

7.7. The effect of RAAS antibody on low density lipoprotein (lDl)

FIG. 89. Plasma LDL level

There is no significant difference on plasma LDL between groups.

7.8. The effect of RAAS antibody on Atherosclerosis plaque lesion area

FIG. 90. Atherosclerosis plaque area

FIG. 91. Percent of plaque area

As shown in figures above, Atorvastatin significant reduced the plaque lesion area in ApoE knockout mice after 8 weeks treatment. RAAS antibody APOA1 low dose showed a trend on reducing the plaque lesion area of aorta in ApoE knout mice after 8 weeks treatment.

FIG. 92. Comparison percent of plaque area in study 1 and study 2.

We also compared percent of plaque area in the study 1 and study 2. In study 1, all ApoE ko mice were fed with HFD for 4 weeks and mice were sacrificed at 14 weeks of age. In study 2, ail ApoE ko mice were fed with HFD for 19 weeks except the mice in negative control group and all mice were sacrificed at 29 weeks of age. Obviously the percentage of plaque lesion area in all groups of mice in study

2 significantly increased than the one in study 1. The model of atherosclerosis in aorta was established successfully.

We analyzed the aortic plaque in different regions as shown in below:

FIG. 93, illustration of analyzing artery regions

Because the total lumen area in arterial arch is very difficult to identify in en face vessel, we measured the total area at the length of about 2 mm from aortic root down to the thoracic artery.

FIG. 94, Root plaque area

FIG. 95, Percent of root plaque area

Atorvastatin and APOA1 mid dose and low dose showed a trend of reducing the arteriosclerosis plaque lesion in the region of thoracic aorta but didn't reach significant difference compared to the vehicle group

FIG. 96, illustration of artery analyzing regions

As shown in the above panel, the total area from the aortic root to the right renal artery was measured.

FIG. 97, results of plaque area from root to right renal

FIG. 98, percent results of plaque area from root to right renal

As shown in the figure above, Atorvastatin showed a trend of reducing the atherosclerosis plaque lesion in the region from the aortic root to right renal artery but didn't reach the significant difference (p=0.08). RAAS antibody APOA1 also showed a trend of reducing the atherosclerosis plaque lesion in a dose dependent manner in this region.

7.9. The effect of aortic inner lumen area and mean density

FIG. 99. Aortic inner lumen area

FIG. 100. Mean density

There is no significant difference on aortic inner lumen area and mean density between the groups.

7.10. The effect of RAAS antibody on liver weight

FIG. 101. Liver weight

FIG. 102. liver weight index

RAAS antibody at the low dose reduced the ratio of liver weight/body weight significantly in ApoE ko mice after 8 weeks treatment compared to the vehicle group. Atorvastatin at 20 mg/kg reduced liver weight and the ratio of liver/body weight significantly in ApoE ko mice after 8 weeks treatment compared to the vehicle group

7.11. The effect of RAAS antibody on fasting overnight blood glucose

FIG. 103. Fasting overnight blood glucose

Atorvastatin and RAAS antibody had no effect on fasting overnight blood glucose after 8 weeks treatment compared to the vehicle group.

7.12. Image of aorta red oil staining

We selected some image of aorta stained by oil red and presented as below. The branches of artery and the lipid plaques could be observed clearly and the plaques mainly distribute in the aortic root and principal branches of the abdominal aorta. It is consistent with the reference literatures.

FIG. 104, Aorta stained by oil red

FIG. 105, Aorta stained by oil red in different groups

Negative control

FIG. 106

Vehicle control

FIG. 107

APOAI high dose

FIG. 108

APOAI medium dose

FIG. 109

APOAI low dose

FIG. 110

Positive control

FIG. 111

### 8. Conclusion

1) Atorvastatin at 20 mg/kg significantly reduced body weight, plasma TC, liver weight and the ratio of liver/BW, the plaque lesion area of aorta in ApoE ko mice after 8 weeks treatment.

2) RAAS antibody APOA1 low dose significantly reduced plasma TC and the ratio of liver/BW in ApoE ko mice after 8 weeks treatment.

3) RAAS antibody APOA1 low dose showed a trend of reducing body weight, plasma TC level, liver weight, the plaque lesion area of aorta in ApoE ko mice fed with HFD continuously for 18 weeks after 8 weeks treatment.

Conclusion of 3 studies on lipid panel:

We have performed the above 3 studies for 4 weeks, 8 weeks and 16 weeks. According to all the previous published studies on ApoE knockout mice the HDL (good cholesterol) and LDL (bad cholesterol) have shown a very disturbing result in the vehicle group, which has higher HDL and lower LDL to compare with the treated groups. When the vehicle which have been fed a HIGH FAT DIET AND CHOLESTEROL for 8 weeks before the injection of the tested AFOD RAAS J (APOAI), and continue to be fed for another 4 weeks, and another 8 weeks and another 16 weeks.

However in comparison with the vehicle control it has shown a decrease in total cholesterol and triglycerides in tested groups.

Final Report of Efficacy Study on AFOD KH in db/db

mice

Study Title: Efficacy study of RL\i\S antibodies on ‘“fype 2 diabetic mouse model in db/db mice

Study Number: CPB-Pll-2504-RAAS

Date: Mar. 28, 2012

### 1. Abbreviations and Definitions

| | |
|---|---|
| kg | kilogram |
| g | gram |
| Mg | milligram |
| ng | Nanogram |
| ml | Milliliter |
| !JL | microliter |
| h | hours |
| n1in | minutes |
| Cpd | Compound |
| BW | Body \1\/eight |
| BG | Blood Glucose |
| FBG | Fasting Blood Glucose |
| DOB | Date of Birth |
| TC | Total Cholesterol |
| TG | Triglyceride |
| LDL | Low Density Lipoprotein |
| HDL | High Density Lipoprotein |
| FB\N | Fasting Blood Glucose |
| Standard Deviation | Standard error Intraperitoneal injection |
| | paraformaldehyde |

### 2. Introduction

The study described in this report evaluated in vivo efficacy of RAAS antibody

### 3. Purpose

To evaluate the efficacy effect of RAAS antibodies 0.1\FOD.′/\FCC and APOi\ ! on blood glucose and related parameters in db . . . 1. db mouse modeL

### 4. Materials

4.1 Compound: AFOD, AFCC, APOA

4.2 Animal: db/db and db/+C57 BLKS

Sex: male

Strain: C57BLKS

Vender: CP in house breeding

Age: 10 weeks (DOB: 26 Aug. 2011} Number: 60 db/db mice and 8 db/m mice

4.3. Glucose test strips: ACCU-CHEK Performa: ROCHE (Lot#470396 2012 Jun. 30)

4.4. CRYSTAL Mouse Insulin ELISA Kit (Cat#90080 Lot#

10NOUMI148, 11NOUM!200)

4.5. Microplate Reader: Spectra Max PLUS384 Molecular Devices

### 5 Experiment Method

5.1. Original Group:

Fasting 6 hours and overnight blood glucose were measured. 60 db/db mice were assigned into 5 grouped based on the fasting 6 h blood glucose and body weight. Two mice with very low body weight were excluded from group. 8 db/rn lean mice was used as negative control group

TABLE 1
the information of groups

| | |
|---|---|
| Vehicles | 12 db/db mice |
| High Dose: | 12 db/db mice |
| Mid Dose: | 12 db/db mice |
| Low Dose: | 12 db/db mice |
| Negativity Control (db/m lean mice) | 8 db/db mice |

5.2. Study duration: This study was conducted in two periods: Period 1: Oct. 13, 2011-Feb. 10, 2012: Test 3 doses of AFOD Period 2: Feb. 13-Mar. 16, 2012: Test 3 antibody products

TABLE 2
The introduction of 2 periods

| | Period 1 | | Period 2 | |
|---|---|---|---|---|
| Antibody | AFOD | | AFOD, AFCC, APOA I | |
| Duration | Nov. 18, 2011-Feb. 10, 2012 | | Feb. 13-Mar. 16, 2012 | |
| | (0-10 wks) | | (10-15 wks) | |
| Group | Vehicles | 12 db/db | Vehicles | 12 db/db |
| | Positive | 12 db/db | Positive | 12 db/db |
| | (Piogiltazone 30 | | (Piogiltazone 30 | |
| | mg/kd/day) | | mg/kd/day) | |
| | High Dose: | 12 db/db | High Dose: | 12 db/db |
| | AFOD | | AFOD | |
| | 1.2 ml l.p | | 0.2 ml l.p | |
| | Mid Dose: AFOD | 12 db/db | Mid Dose: AFOD | 12 db/db |
| | 1.0 ml I.p | | 0.2 ml I.p | |
| | Low Dose: | 12 db/db | Low Dose: | 12 db/db |
| | AFOD | | AFOD | |
| | 0.8 ml I.p | | 0.2 ml I.p | |
| | Negative Control | 8 db/+ | Negative Control | 8 db/+ |
| | (db/m lean mice) | | (db/m lean mice) | |

Treatment 8 dose
Note:
5 mice died during the 11 weeks study period and their BW decrease significantly after AFOD injection

Timeline

Period 1: Oct. 13, 2011-Feb. 10, 2012;

Nov. 18, 2011: Measure fasting overnight blood glucose and body weight

Nov. 21, 2011: Measure fasting 6 h blood glucose and body weight.

Nov. 23, 2011: Fasted overnight and co!lect the blood plasma for insulin test before the treatment.

Nov. 28, 2011: Group the mice based on the fasting 6 h blood glucose and fasting body weight and start the treatment with 3 doses of antibody AFOD by i.p every two days (Monday, Wednesday, and Friday).

Dec. 16, 2011-Feb. 10, 2012: Stop all the treatment including the positive control group.

Nov. 28, 2011-Feb. 10, 2012: Measure body weight and blood glucose weekly.

Jan. 13, 2012& Feb. 9, 2012: Weigh the body weight and collect blood p!asrna for insulin measurement (fasted overnight).

Period 2: Feb. 13-Mar. 16, 2012:

Feb. 13, 2012: Start the treatment with 3 antibodies by i.p every two days (Monday, Wednesday, Friday) after 8 weeks washout from previous treatment.

Feb. 13-Mar. 16, 2012: Measure body weight and blood glucose weekly.

Mar. 13, 2012: Weigh body weight and collect the fasting overnight blood plasma for insulin measurement.

Mar. 16, 2012: Sacrific the mice and collect the plasma for lipid profile measurement, measure the body and liver weight, and collected pancreas by fixing in the 4% paraformaldehyde.

5.3. Route of compound administration:

Antibody products were administrated by intraperitoneal injection and the positive compound was mixed into food at the dose 30 rng/kg/day.

5.4. Body weight and blood glucose measurement: Fasting 6 hours

body weight and blood glucose concentration were measured by Roche giucometer weekly.

5.5. Plasma insulin measurement: About 30 ul of blood sample was collected from the orbital vein for each mouse and centrifuged 7000 rpm

at 4° C. for 5 min. Plasma samples were saved in −70 l-::. The plasma insulin level was measured with EUSA kit (CRYSTAL, cat#90080),

5.6. Plasma lipid profile measurement: The plasma lipid profile were measured by the DaAn Clinic central lab.

5.7. Study taken down: After 14 dose antibody products treatment, all mice were sacrificed. Measure body weight and collect blood sample for each mouse. Measure liver weight and save one piece for pathology study and freeze one piece in liquid nitrogen for further analysis in the future. Save pancreas into 4% paraformaldehyde (PFA) fixation solution for future analysis.

5.7. Clinic observation: Several mice lost body weight significantly after AFOD treatment as shown in the results. Total 7 mice from different groups died during the 4 months study period due to kidney infection or skin ulcer or skin abscess. The information of dead animals was shown in the table below and the more detail information about dead mice was listed on the sheet of dinic observation of raw data file.

TABLE 3
The information of dead mice

| | Part 1 | | | | | | | |
|---|---|---|---|---|---|---|---|---|
| | low | | | | Part 2 | | | |
| | blood | kidney | lung | No | kidney | intestinal | kidney | Total |
| Group | glucose | infection | bleeding | reason | infection | bump | bleeding | 11 |
| Vehicle | | | | | | | | 0 |
| high dose | 1 | | | 1 | 2 | | | 4 |
| mid dose | | | | | 2 | | | 2 |
| low dose | | 1 | 1 | | | 1 | | 3 |
| Positive | | 1 | | | | | 1 | 2 |
| group | | | | | | | | |
| Negative | | | | | | | | 0 |
| control | | | | | | | | |

### 6. Data Analysis

The results were expressed as the Mean±SEM and statistically evaluated by student's t-test. Differences were considered statistically significant if the P value was <0.05 or <0.01.

### 7. Results

PART 1: Nov. 18, 2011-Feb. 10, 2012 (0-10 weeks)

7.1.1. Effect of AFOD on body weight

FIG. 112, Body weight

AFOD at 3 doses significantly reduced body weight in db/db mice after 3 weeks treatment compared with vehicle group but the difference disappeared after the treatment stopped from week 4. The Positive control Pioglitazone significantly increased body weight in db/db mice after 2 weeks treatment but lost difference after the treatment stopped.

7.1.2. Effect of products on blood glucose (Fasting 6 h).

FIG. 113. Blood glucose (Fasting 6 h)

As shown in FIG. 2, positive control Piog!itazone significantly reduced blood glucose in db/db mice after 1 week treatment and blood glucose level was back to vehicle group levels 10 days after treatment stop. AFOD at low dose showed the effect on lowering blood glucose after 8 doses treatment.

7.1.3. Effect of products on fasting overnight BG

FIG. 1.14. Fasting overnight BG

AFOD has no effect on fasting overnight BG in db/db mice but the positive control Pioglitazone can significantly lower blood glucose after 1 week treatment and blood glucose level back to the vehide control levels gradually after the treatment stopped.

7.1.4. The effect of AFOD on plasma insulin and HOMA-IR

FIG. 115. Plasma insulin

FIG. 116 HOMA-IR

As shown in FIGS. 4A and 4B, AFOD at low dose showed a trend on reducing plasma insulin level and improving insulin resistance in db/db mice after 8 doses treatment.

PART 2: Feb. 13-Mar. 16, 2012

7.2.1. The effect of AFODAFCCAPOA I on body weight

FIG. 117. The effect of AFOD, AFCC, APOA I on body weight

Three products have no effect on body weight in db/db mice compared to vehicle group but the positive control pioglitazone showed an effect on increasing body weight. 7.2.2. The effect of AFOD, AFCC, APOA I on fasted 6 h blood glucose

FIG. 118. Blood glucose (fasted 6 h)

There is significant difference on blood glucose between the pioglitazone group and vehide group but the three test artic!es” showed no effect on fasting 6 h blood glucose.

7.2.3. The effect of three products on overnight fasting blood glucose

FIG. 119. Blood glucose (fasted overnight}

Three antibody products had no effects on overnight fasting blood glucose in db/db mice compared to the vehicle group. but positive control piog!itazone significantly reduced the fasting overnight blood glucose level after 4 weeks treatment in db/db mice.

7.2.4. The effect of three products on plasma insulin and HOMA-IR

FIG. 120. Plasma insulin

FIG. 121. HOMA-IR

AFOD showed a trend on improving plasma insulin resistance in db/db mice after 14 doses treatment (p=0.054), the pioglitazone also showed an trend on improving insulin resistance after 5 weeks treatment in aging db/db mice at 6 months old (p=0.051).

7.2.5. The effect of AFOD, AFCC, APOA I on plasma lipid

FIG. 122. Plasma lipid profile

Three antibody products have no effects on plasma lipid profile in db/db mice after 14 doses treatment compared to the vehicle group; but positive control pioglitazone significantly lowered the p!asma triglyceride !evel in db/db mice after

5 weeks treatment.

7.2.6. The effect of AFOD, AFCC, APOA I on liver weight

FIG. 123. Liver weight

Three antibody products have no effect on liver weight and the ratio of liver/body weight compared to the vehicle group. The positive control pioglitazone showed the effect on reducing the ratio of !iver weight to body weight due to the increase of body weight.

7.2.7. Plasma insulin level in db/db mice during two periods of study

FIG. 124. Four measurements of plasma insulin

The plasma insulin level in db/db mice were gradually declined when mice are getting older.

### 8. Conclusion

Study period 1:

,;.; . . . Positive control piog!itazone significantly reduced the blood glucose !eve! and increased body weight after 1 week treatment in db/db mice compared to the vehicle group. Both b!ood glucose and body weight in this group of mice gradually went back to baseline after the treatment stopped.

Y AFOD at three doses reduced the body weight significantly after 3 weeks

treatment in db/db mice compared to the vehicle group. AFOD at low dose (0.8 ml i.p injection, q.o.d) showed a trend on lowering blood glucose and improving insulin resistance compared to the vehicle.

Study period 2:

? The positive control pioglitazone has follow effects in db/db mice after 4 weeks treatment:

../ lower blood glucose (Fasted 6 h and overnight)

../ increase body weight

./. reduce plasma triglyceride level

../ improve the insulin resistance

? RAAS product AFOD at low dose showed a trend on improving insulin resistance in db/db mice after 4 weeks treatment (14 doses i.p. injection) but didn't reach the statistic difference (p=0.054) compared to the vehicle group.

In Vivo Efficacy Testing of eight RAAS compounds in 411-1UC Breast Cancer Cell Orthotopic Model

Apr. 25, 2012-Jun. 28, 2012

Table of Contents

| 1, OBJECTIVE | 1 i2 |
|---|---|
| 2. l'v1ATERJALS AND tv1ETHOD | 1 i2 |
| 2.1. Animals, reagents and instruments | 112 |
| 2.1.1 Animal Specifications | 112 |
| 2.1.2 Animal Husbandry | 1.12 |
| 2.1..3 Animal procedure | 113 |
| 2.1.4 Reagents and instruments | 113 |
| 2.2. Procedure and n1ethod | 113 |
| 2.2.14 T1-i.UC cell culture | 113 |
| 2.2.1.1 4TJ-LUC cell1haw | 113 |
| 22.L2 Subcui1ure oftbe 4Tl-·luc cells | 114 |
| 2.2.1.3 Harvest of 4Tl-luc cells | 114 |
| 2.2.2 Animal model establishment | 114 |
| 2.2.3 rv1easurements | 1.15 |
| 2.2.4 Formulation preparation | 115 |
| 2.2.4.1 Compotmds preparation: | 115 |
| 2.2.4.3 Gemcitabine solution preparation: | 115 |
| 2.2.5 Animal experiment | 1.16 |
| 2.2.5.1 Random assignment of treatment groups | 116 |
| 2.2.5.2 Administration of the animals | 116 |
| 2.2..6 Experimental endpoint | 117 |
| 2..3 Statistical Analysis | 1.17 |
| 2.3.1 TGI (tumor grmNth inhibition, in percentage) | 117 |
| 2.3.2 T/C (!,)calculation | 117 |
| 2..3.3 ANOVA analysis | 1.17 |

.:.<- Jl1T.:i.!. 1::!.R..1.I.:;K;,h 0JQ L.<::.<:: .:: .::.::o.<!!.<!!.:: .::.::o.<!!.<!!.:: .::.::o.<!!.<!!
.:: .::.::o.<!!.<!!.:: .::.::o.<!!.<!!.:: .::.::o.<!!.<!!.:: .::.1.t .

| | |
|---|---|
| 3.1 Tumor growth curve based on relative ROI | 118 |
| 3.2 Tumor growth curve based on tumor volume | 118 |
| 3.3 Toxicity evaluation by body weight change(%) monitoring and daily | 1.19 |
| observation of 4Tl-l.UC-bearing Balb/c nude mice | |
| 3.4 TGI (%)calculation | 120 |
| 3.5 T/C (%)calculation | 121 |
| 4. CONCLUSION | l21 |
| APPENDICES | 122 |
| EXHIBIT 1: FLUORESCENCE IMAGES OF THE WHOLE BODY | 122 |
| EXHIBIT 2: RELATIVE ROl, TUMOR VOLUME AND BODY WEIGHT | 123 |
| EXHIBIT 3: DAILY TESTING ARTICLES RECORD | l47 |

EXECUTIVE SUMMARY

Effects of AFOD RAAS 1/8, AFOD RAAS 2, AFOD RAAS 3, AFOD RAAS 4, AFOD RAAS 5, AFOD RAAS 6, AFOD KH and AFCC KH on tumor growth in Balb/c nude mouse orthotopic model from 4T1-LUC cell line were investigated in this study. Toxicity was evaluated by body weight monitoring as well as daily observation. Bioluminescence was measured with !VIS Lumina !! machine. Mice treated with AFOD RAAS 1/8, AFOD RAAS 2, AFOD RAAS 3, AFOD RAAS 4, AFOD RAAS 5, AFOD RAAS 6, AFOD KH and AFCC KH exhibited a significant reduction of Relative ROI 6 and 9 days after compounds administration, as compared to vehicle control.

During the first 16 days post administration (Day 1 to Day 16L body weight of all of the testing article and gemcitabine treated mice, got increased stably, which indicated that both the testing compounds and control agent gemcitabine were well tolerated at this stage by current dosing schedule. However, significant body weight loss was found in testing article treated mice since Day 17 and the situation got even worse on Day 22 probably because dosing volume changed from 0.4 ml/mouse to 0.6 ml/mouse on that day. As the dosing schedule was changed to 1.0 ml/mouse BID on Day 23, dramatic body weight loss was continuously observed. Macroscopically, all the mice in the testing article treated groups suffered from serious abdomen swelling, so administration was halted for 4 days (Day 25 to Day 28L and the remaining mice were monitored closely. During the experimental period (Day 1 to Day 28) totally 42 mice died, significant body weight loss was found before death. On Day 29, the recovered mice in AFOD RAAS 3 and AFOD RAAS 5 treated groups were IP treated with 0.4 ml/mouse, while the other mice in AFOD RAAS 4, AFOD KH and AFCC KH groups were kept untreated due to bad status. In addition, mice in gemcitabine group were monitored by IVIS after stop

dosing. The results indicated that although the testing compounds might have potential anti-tumor effect, dose, schedule and route of administration were also Important for validation of such effect.

### 1. Objective

Detennine the effects of AFOD RAAS 1/8, AFOD RAAS 2, AFOD RAAS 3, AFOD RAAS 4, AFOD RAAS 5, AFOD RAAS 6, AFOD KH and AFCC KH on primary tumor growth and metastasis in Balb/c nude mouse orthotopic model established from 4T1-luc breast cancer cells.

### 2. Materials and Method

2.1. Animals, reagents and instruments

Species: Mus Musculus Strain: Balb/c nude mouse Age: 6-8 weeks

Sex: female

Body weight: 18•20 g

2.1.1 Animal Specifkations

Number of animals: 80 mice plus spare

2J 0.2 Animal Husbandry

The mice were kept in laminar flow rooms at constant temperature and humidity with 3 or 4 animals in each cage.
Temperature: 20 2.5 ′C.
Humidity: 40-70%.
   Light cycle: 12 hours light and 12 hours dark.

Cages: Made of polycarbonate. The size is 29 em×17.5 ern×12 cm (L×W×H). The bedding material is wood debris, which is changed once per week.

Diet: Animals had free access to irradiation sterilized dry granule food during the entire study period. Water: Animals had free access to sterile drinking water.

Cage identification: the identification labels for each cage contained the following information: number of animals, sex, strain, date received, treatment, study number, group number, and the starting date of the treatment.

Animal identification: Animals were marked by ear punch.

2.1.3 Animal procedure

i\11 the procedures related to animal handling, care, and the treatment in this study were performed according to guidelines approved by the Institutional Animal Care and Use Committee (IACUC) of WuXi AppTec, following the guidance of the Association for Assessment and Accreditation of Laboratory Animal Care (AAALAC). At the time of routine monitoring, the animals were checked and recorded for any effects of tumor growth on nonnal behavior such as mobility, food and water consumption (by looking only), body weight gain/loss, eye/hair matting and any other abnormal effect.

2. L4 Reagents and instruments

4T1-LUC cell line (Caliper, USA); RPIV11 1640 medium (Invitrogen, USA); FBS (Invitrogen, Australia); DPBS (Fisher, USA); PBS (Gibco, USA); Sodium-Heparin (Sigma, USA); l′v1C (Sigma, USA); Formaldehyde (Sinopharm, China); Twelve-hydrated isodiurn hydrogen phosphate (Sinopharm, China); Sodium dihydrogenphosphate (Sinopharm, China); C02 Incubator (Thermo Scientific, USA); Biological Safety Cabinet (BSC-A 2, Shanghai, China); Centrifuge (Eppendorf, USA); Centrifuge (Thermo Scientific, USA); Pipettor (Thermo Scientific, USA); Finnpipettor (Eppendorf Research, USA); Pipette (Corning, USA); Plastic Cell Culture Flask (Corning, USA); Tube

(Greiner Bio-one, Germany); Microscope (Nikon, Japan); Parafilm (Parafilm M, USA); Electronic

Analytical Balance (Sartorius, Germany); Barnstead Nanopure (Thermo Scientific, USA); Cryopreservation of refrigerator (Haier, China).

2.2. Prm educe and method

2.2. L14T1-LUC cell thaw

0.2.1 4T1-LUC cell culture

One tube of 4T1-1.UC (from Caliper) cells were thawed according to the following procedure:

1. Cells were thawed by gentle agitation of vial in a 37″C water bath. To reduce the possibility of contamination, the 0-ring and cap were kept out of the water. The whole process should be rapid (approximately 2 minutes);

2. Vials were removed from the water bath as soon as the contents were thawed, and was decontaminated by spraying with 7.5% ethanoL All the operations from this point on should be carried out under strict aseptic conditions;

3. The content of the vials was transferred into a centrifuge tube containing 10 ml of complete culture medium (RPMI1640+10% FBS) and was spin at 1000 rpm for 3 minutes. Supernatant was discarded;

4. Cell pellet was resuspended with the 5 ml of medium. The suspension was transferred into a 17.5 cm2

flask, 2.5 ml of complete culture medium was added and mixed;

5. Cells were incubated at 37° (, 5% C0 2.

2.2.1.2 Subculture of the 4T1-Iuc cells

4T1-Iuc cells were split according to the following procedure:

1. Cells were aspirated by gently pipetting;

2. 1 ml of the cell suspension was added into a new 175 en} flask, 30 ml of complete culture medium was added and the flask was gently shaked to spread the suspension throughout the bottom. The subculture ratio was 1:10;

3. Cells \Nere observed under an inverted microscope and were incubated at 3FC, 5% C02.

2.2.1.3 Harvest of 4T1-Iuc cells

4T1-luc cells were harvested according to the following procedure:

1. Cells were harvested in 90% confluence and viability was no less than 90%. 4T1..luc cells were transferred into a conical tube and centrifuged at 1000 rpm for 6 min, supernatant was discarded;

2. Cells were rinsed with 50 ml of PBS twice, the viable cells were counted on a counter, 14×10 7 cells were obtained;

3. 14 ml of PBS was added to make a cell suspension of 10×106 cells/ml and mixed.

2.2.2 Animal model establishment

A total number of 92 female Balb/c nude mice were purchased. These mice were allowed 3 days of acclimatization period before experiments start.

The cell suspension was carried to the animal room in an ice box. 100 fiL of 1×106 4T1-luc cells was implanted orthotopiclly into the right rear mammary fat pad lobe of each mouse. Totally 80 mice were selected and divided into 10 groups. All mice were monitored daily.

2.2.3 Mea:sun.'nH.'nts

Tumor growth status was monitored by both IVIS Lumina II and a digital caliper twice weekly since the day after cell implantation.

2.2.3.1ROI (region of interest) measurement.

For IVIS Lumina II measurement, bioluminescence intensity of primary tumor and metastatic tumor was obtained according to the following procedure:

1. Tumor-bearing mice were \Neighted and intra peritoneally administered luciferin at a dose of 150 mg/kg (10 ml/kg);

2. After 10 min, mice were pre-anesthetized with the mixture of oxygen and isoflurane. When the animals were in complete anesthetic state, move them into the imaging chamber and obtain bioluminescence images with IVIS machine (Lumina II);

3. ROI data was calculated with IVIS Lumina II software and relative ROI was calculated to express the tumor growth status.

Relative ROI::: ROit/ROI1, where ROI,--ROI value at day t ROI1 . . . ROI value at day 1

2.2.3.2 Tumor volume measurement

Tumor size was measured twice a week in two dimensions using a caliper. and the tumor volume (V) was expressed in mm3 using the formula: V=0.5 a×b2 where a and bare the long and short diameters

of the tumor, respectively.

2.2.4.1 Compounds preparation:

2.2.4 Formulation preparation

(1) AFOD RAAS 1/8, AFOD RAAS 2, AFOD RAAS 3, AFOD RAAS 4, AFOD RAAS 5, AFOD RAAS 6, AFCC KH

solutions were provided by client and stored at 4° C.

2.2.4.2 AFOD KH solutions were filtered with Millipore membrane filters before dosing.

2.2.4.3 Gemcitabine solution preparation:

200 mg gemcitabine was dissolved in 33.3 ml 0.9% NACL. and vortexed to obtain 60 mg/ml gemcitabine solution.

2.2.5 Animal experiment

2.2.5.1 Randorn assignment of treatment groups

8 days post 4Tlinoculation, when tumors reached an average volume of 79 mm 3 80 out of the 88 mice

were selected based on relative ROI and tumor volume. These animals were randomly assigned to 10 groups (n=8).

2.2.5.2 Administration of the animals

1. 1\/lice were treated with AFOD RAAS 1/8, AFOD RAAS 2, i\FOD RAAS 3, AFOD RAAS 4, AFOD RAAS 5, AFOD RM\S 6, AFOD KH, AFCC KH and gemcitabine since Random assignment according to Table 1. The first administration day was denoted as Day 1.

TABLE 1
Experimental design

| | Dosage | | Dosing | Animal |
|---|---|---|---|---|
| Treatment | (ml/mouse) | Dosing Route | Schedule | Number |
| Control | n/a | n/a | n/a | 8 |
| Gerncita bine | 60 mg/kg | IP | 2X/WK | 8 |
| AFOD RAAS 1 | 0.2/0.4 | 1V/IP | OD | |
| AFOD RAAS 2 | 0.2/0.4 | 1V/IP | OD | |
| AFOD RAAS 3 | 0.2/0.4 | 1V/IP | OD | |
| AFOD RAAS 4 | 0.2/0.4 | IV/IP | QD | |
| AFOD RAAS 5 | 0.2/0.4 | IV/IP | QD | |
| AFOD RAAS 6 | 0.2/0.4 | IV/IP | QD | |
| AFOD KH | 0.2/0.4 | IV/IP | QD | |
| AFCC KH | 0.2/0.4 | IV/IP | OD | |

Note: 1. Animals in vehicle group did not receive any treatment.

2. For every administration group, detailed dosing information could be found in Exhibit 3.

2. Mice were observed daily to identify any overt signs of adverse, treatment-related side effects of compounds, any upset and uncomfortable of mice were recorded. Body weights were measured and recorded twice weekly.

2.2.6 Experimental endpoint

1. On Day 31(39 days post inoculation), all animals in vehicle group died.

2. On Day 35 (43 days post inoculation), all AFOD RAAS: 1/8, AFOD Ri\AS 2, AFOD RAAS

3, i\FOD RAAS 4, AFOD RAAS 5, AFOD RAAS 6, AFOD KH, AFCC KH treated animals died.

3. Animals in gemcitabine group are monitored by IVIS after stop dosing.

2.3 Statistical Analysis

2.3.1 TGI (tumor growth inhibition, in percentage)

TGI (tumor growth inhibition, in percent) was calculated according to the following equation:

TGI (%)={1−(Tl−TO)/(C1 . . . CQ)}, where

Cl—median tumor volume of control mice at timet T:l—median tumor volume of treatment mice at timet CO—median tumor volume of control mice at time 0

TO—median tumor volume of treatment mice at time 0

2.3.2 T/C (%). alculation

T/C (%) was calculated based on the tumor volume data collected on Day 27.

2.3.3 AN OVA analysis

The difference between the mean values of tumor volume in treatment and vehicle groups was analyzed for significance using one way ANOVA test at each time point after log transformation.

### 3. Results and Discussion

3.1 Tumor growth curve based on relative ROJ

FIG. 1 showed the relative ROI changes after administration of vehicle, gemcita bine and AFOD RAAS

1/8, AFOD RAAS 2, AFOD RAAS 3. AFOD RAAS 4, AFOD RAAS 5, AFOD RAAS 6, AFOD KH, AFCC KH. As shown in Table 2. no significant changes in relative ROI were found in all AFOD RAAS 1/8, AFOD RAAS 2, AFOD RAAS 3, AFOD RAAS 4. AFOD RAAS 5, AFOD RAAS 6, AFOD KH, AFCC KH treated groups as compared to vehicle group. The bioluminescence graphs and the relative ROI values were displayed in Exhibit 1 and Exhibit 2.

FIG. 125

FIG. 126

FIG. 127

FIG. 1 Relative ROI changes of 4T1-LUC-bearing BALB/C nude mice after administration of vehicle, AFOD RAAS 1/8, AFOD RAAS 2, AFOD RAAS 3, AFOD RAAS 4, AFOD RAAS 5, AFOD RAAS 6, AFOD KH, AFCC KH, and Gemcitabine. Data were shown as mean±SEM. Mean value and SEM was calculated based on survived animals.

TABLE 2
Summary of one-way ANOVA analysis on relative ROI changes

| | | | AFOD | | | | | | | |
|---|---|---|---|---|---|---|---|---|---|---|
| | Genteitabine | | RAAS | AFOD | AFOD | AFOD | AFOD | AI OD | AFOD | |
| | 60 mpl (ip | | 1/8 | RAAS 2 | RAAS | RAAS 4 | RAAS 5 | RAAS 6 | KM | AFCC |
| Day | 2X/IWK | | QFS | QD | :3 QD ¢ | QD | QD | QD | QD | KM QD |
| | ** | NS | | NS | NS | NS | NS | NS | NS | NS |
| 16 | 13 | NS | | NS | NS | NS | NS | NS | NS | NS |
| | * | NS | | NS | NS | NS | NS | NS | NS | NS |
| | ** | | | | | | | | | |
| 20 | *** | NS | | Ts | NS | NS | NS | NS | NS | NS |
| 73 | *** | NS | | NS | NS | NS | NS | NS | NS | NS |
| 27 | *** | A | NS | NS | NS | NS | NS | NS | NS | NS |

3.2 Tumor growlb curve based on tumor volume

FIG. 2 showed the tumor volume changes of 4T1-LUC-bearing Balb/c nude mice after administration of vehicle, AFOD RAAS 1i8, AFOD RAAS 2, AFOD RAAS 3, AFOD RAAS 4, AFOD RAAS 5, AFOD RAAS 6, AFOD KH, AFCC KH, and gerncitabine.

No significant tumor volume reduction was observed in all AFOD RAAS 1/8, AFOD RAAS 2, AFOD RAAS 3, AFOD RAAS 4, AFOD RAAS 5, AFOD RAAS 6, AFOD KH, AFCC KH treated groups when compared to vehicle group, while gerncitabine exhibited significant tumor volume reduction role since day 13 after administration as compared to vehicle control. (Table 3).

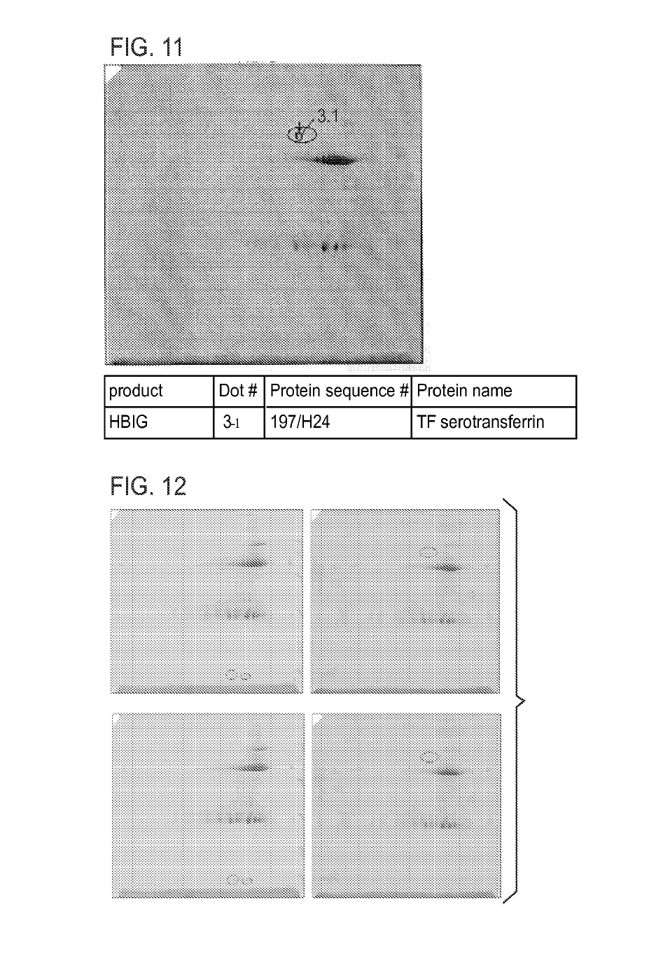

FIG. 12.8

FIG. 129

FIG. 130

FIG. 2 Tumor volume changes of 411-LUC-bearing Ba!b/c nude mice after administration of vehide, AFOD RAAS 1/8, AFOD RAAS 2, AFOD RAAS 3, AFOD RAAS 4, AFOD RAAS 5, AFOD RAAS 6, AFOD KH, AFCC KH, and Gemdtabine. Data were shown as mean±SEM. Mean value and SEM was calculated based on survived animals.

TABLE 3
Summary of one-way ANOVA analysis on tumor volume changes

| | Gemcitabine | | AFOD | AFOD | AFOD | AFOD | AFOD | AFOD | AFOD | AFCC |
|---|---|---|---|---|---|---|---|---|---|---|
| | 60mpkip | | R1\AS | Rr\AS 2 | Riv\S 3 | RAAS4 | Rr\i\S5 | RAAS6 | KH | KH |
| Day | 2X/WK | | 1/8 QD | QD | QD | QD | QD | QD | QD | QD |
| 10 | NS | NS | | NS | NS | NS | NS | NS | NS | NS |
| 13 | ** | NS | | NS | NS | NS | NS | NS | NS | NS |
| 16 | *** | NS | | NS | NS | NS | NS | NS | NS | NS |
| 20 | *′** | NS | | NS | NS | NS | NS | NS | NS | NS |
| ′) | *** | NS | | NS | NS | NS | NS | NS | NS | NS |
| .,;_.) | | | | | | | | | | |
| 27 | *** | NS | | NS | NS | NS | NS | NS | NS | NS |

3,3 Toxidty evaluation by body weight change (′;) monitoring and daily observation of 4T1-LUC-

bearing Balb/c nude mice

Body weight change (%) is one of the important indicators to exhibit the toxicity of the testing materials. FIG. 3 showed the body weight change (%) during the whole study period

(Exhibit 2.). During the first

16 days post administration (Day 1 to Day 16), body weight of mice in all of the testing article and gemcitabine treated groups increased normally, implying that the compounds were well tolerated via current dosing schedule. However, the body weight loss was found since Day 17 and the situation got even worse on Day 22 by changing dosing volume from 0.4 mlimouse to 0.6 ml/mouse and then to 1.0 ml/mouse BID on Day 23. Macroscopically, all the mice in the testing article treated groups suffered from serious abdomen swelling, so administration was halted for 4 days (Day 25 to Day 28), and the remaining mice were monitored closely. During the experimental period totally 42 mice died, significant body weight losses were found before mouse death. On Day 29, the recovered mice in AFOD RAAS 3, AFOD RAAS 5 were IP treated with dosing volume of OAml/mouse, while the other mice In AFOD RAAS 41 AFOD KH and AFCC KH groups were kept untreated due to bad status.

Furthermore, mice in gemcitabine group were monitored by IVIS after stop dosing. It seemed that both

the dosing concentration and volume of AFOD RAAS 1/8, i\FOD RAAS 2, AFOD RAAS 3, AFOD RAAS 4,

AFOD RAAS 5, AFOD RAAS 6, AFOD KH, AFCC KH contributed to the deaths. All of the primary tumors of dead mice were removed and weighed.

FIG. 131; FIG. 132; FIG. 133

FIG. 3 Body weight change (%) of 4T1-LUC-bearing Baib/c nude mice following administration of vehicle, gemcitabine and AFOD RAAS 1/8, AFOD RAAS 2. AFOD RAAS 3, AFOD RAAS 4, AFOD RAAS S, AFOD RAAS 6, AFOD KH, AFCC KH. Data were shown as mean±SEM. Mean value and SEM was calculated based on survived animals.

3.4 TGI (%) (alculation

Table 5 showed the tumor grmNth inhibition (TGI) ratio of treatment groups.

TABLE 5
Tumor growth inhibition of four treatment groups

| | | | | Day | Day | Day |
|---|---|---|---|---|---|---|
| TGI (%) | Day 10 | Day 13 | Day 16 | 20 | 23 | 27 |
| Ger eitabine 60mpk ip | 1.09 | 1.14 | 0.95 | 0.88 | 0.96 | 0.99 |
| vs Vehicle | | | | | | |
| AFOD RAAS 1/8 ip | Vs | −0.52 | −0.35 | −.39 | −0.20 | −0.08 |
| | Vehicle, | | | | | |
| | −0.38 | | | | | |
| AFOD RAAS 2 ip | 0.23 | −0.38 | −0.35 | −0.45 | −0.26 | −0.06 |
| Vs Vehicle | | | | | | |
| AFOD RAAS 3 ip | −0.59 | −0.72 | −0.36 | −0.07 | −0.11 | 0.12 |
| Vs Vehicle | | | | | | |
| AFOD RAAS 4 ip | −0.04 | −0.08 | 0.11 | −0.03 | −0.16 | 0.00 |
| Vs Vehicle | | | | | | |
| AFOD RAAS 5 ip | 0.45 | 0.02 | 0.16 | 0.29 | 0.35 | 0.30 |
| Vs Vehicle | | | | | | |
| AFOD RAAS 6 ip | −0.22 | −0.39 | −0.34 | −0.09 | 0.11 | 0.14 |
| Vs Vehicle | | | | | | |
| AFOD kh ip | 0.05 | 0.27 | −0.07 | 0.15 | 0.21 | 0.38 |
| Vs ′Vehicle | | | | | | |
| AFCC kh ip | | | | | | |

3.5 T/C (%) cakulation

T/C (%) was calculated based on the tumor volume data collected on Day 27.

AFOD RAAS 1/8 IP, QD group: T=824.09 mm 3

C=768A7 mm3. T/C (%)=1.07

AFOD RAAS 2. IP, QD group: T=mm 3

C::: 768.47 mm3. T/C (%)=1.06

AFOD RAAS 3 IP, QD group: T::: 686.52 mm 3, C=768.47 mm3. T/C (%)::: 0.89

AFOD RAAS 4 IP, QD group: T=770.20 mm 3

C=768.47 rnm3. T/C (%)::: 1.00

AFOD RAAS 5 IP, QD group: T=564.66 mm 3

C::: 768.47 mm3. T/C (%)=0.73

AFOD RAAS 6 IP, QD group: T=672.66 mm 3, C=768.47 mm3. T/C (%)=0.88

AFOD KH IP, QD group: T 506.57 mm 3 C::: 768A7 mm3. T/C (%) 0.66

AFC:C: KH IP, QD group: T=690.57 mm3

C::: 768.47 mm3. T/C: (%)=0.90

### 4. Conclusion

Effects of AFOD RAAS 1/8, AFOD RAAS 2, AFOD RAAS 3, AFOD RAAS 4, AFOD RAAS 5, AFOD RAAS 5, AFOD KH, AFCC KH on tumor growth in Balb/c nude mouse orthotopic model from 411-LUC cell line were investigated in this study. Toxicity was evaluated by body weight monitoring as well as daily observation. Bioluminescence was measured with IVIS Lumina II machine. The results indicated that no significant change in relative ROI as well as in tumor volume was found in all test treated groups as compared with vehicle group.

In this study, we found out that continuous administration of all of the testing articles, including AFOD RAAS: 1/8, AFOD RAAS 2, AFOD RAAS 3, AFOD RAAS 4, AFOD RAAS 5, AFOD RAAS 6, AFOD KH and AFCC KH could render dramatic weight loss, although this is not obvious during the first 16 days post

treatment, Notably, all the testing article treated mice suffered from serious abdomen swelling. Take together, the results indicated that although the testing compounds might have potential anti-tumor effect, dose, schedule and route of administration were also important for validation of such effect.

### APPENDICES

Exhibit 1: fluorescence images of the whole body

FIG. 134

FIG. 135

Exhibit 2: Relative ROI, tumor volume and body welrght

| | | 4T1-hue Relative ROI | | | | | | | | | | | | |
|---|---|---|---|---|---|---|---|---|---|---|---|---|---|---|
| | | (photosisecond) | | | | | | | | | | | | |
| | | 2012 | 2012 | | | | | | | | | | | |
| | | May | May | | | 2012 | 2012 | 2012 | 2012 | 2012 | 2012 | 2012 | 2012 | 2012 |
| | An | 9 | 11 | 2012 | 2012 | May 21 | May 24 | May 28 | May 31 | Jun. 4 | Jun. 9 | Jun. 11 | Jun. 14 | Jun. 18 |
| Gra | mai | Day | Day | May 14 | May 17 | Day | Day | Day | Day | Day | Day | Day | Day | Day |
| up | ID | 1 | 3 | Day 6 | Day 9 | 13 | 16 | 20 | 23 | 27 | 32 | 34 | 37 | 41 |
| | 1 | 1.00 | 6.52 | 15.68 | 26.4 | 68.68 | 103.90 | 145.84 | 126.93 | 263.97 | 250.672 | | 496.030 | |
| | | | | | 43.41 | | | | | | | | | |
| | 2 | 1.00 | 6.25 | 66.33 | 211. | 360.04 | 405.269 | 821.37 | 1054.4 | 2518.04 | 2510.341 | | 1169.20100 | |
| | | | | | 308.08 | | | | | | | | | |
| | 3 | 1.00 | 29.04 | 131.01 | 354. | 838.92 | 1155.19 | 1516.47 | 1154.69 | 20 | | | | |
| | | | | | 787.96 | | | | | | | | | |
| a= | 4 | 1.00 | 4.63 | 23.94 | 36.9 | 82.76 | 161.37 | 291.25 | 589.70 | 8 | | | | |
| | | | | | 40.03 | 72.00 | | | | | | | | |
| t> | 5 | 100 | 13.63 | 52.99 | 179. | 487.58 | 518.686 | 663.41 | 1345.3 | 1583.06 | 1681.674 | 34 | | |
| | | | | | 408.85 | | | | | | | | | |
| | 6 | 1.00 | 18.43 | 79.41 | 117. | 219.28 | 380.460 | 702.60 | 867.35 | 764.11 | 574.298 | 732.047 | 8 | |
| | | | | | 210.51 | | | | | | | | | |
| | 7 | 1.00 | 2.20 | 29.94 | 33.4 | 65.36 | 159.55 | 225.54 | 185.33 | 278.01 | 219.400 | 3 | | |
| | | | | | 47.18 | | | | | | | | | |
| | 8 | 1.00 | 20.20 | 79.39 | 111. | 280.99 | 607.567 | 668.72 | 784.90 | 598.26 | 698.548 | 9 | | |
| | | | | | 122.46 | | | | | | | | | |
| Mean | | ′ 00 | 12.61 | 59.84 | 133. | 300. | 425.31447 | 613.14 | 726.19 | 942.33 | 3 | | 799.09 | |
| | | | | | 246.96 | 989.06 | | | | | | | | |
| Std. | | 0.35 | 3.56 | 21.09 | 40.4 | 88.4 | 124.2823 | 235.4. | 292.08 | 226.13 | | | 422.65 | |
| Err. | | | | | 58.98 | 259.4 | | | | | | | | |
| | 1 | 1.00 | 10.69 | 18.02 | 10.0 | 23.0 | 16.588 | 27.01 | 90.68 | 73.238 | | | 81.27 | |
| | | | | | 24.00 | 20.031 | | | | | | | 55.3427 | |
| | 2 | 1.00 | 2.07 | 15.37 | 5.24 | 14.0 | 8.437 | 10.00 | 11.41 | 29.854 | | | 20.75 | |
| | | | | | | 12.8 | | | | | | | 43.0606 | |
| | | | | | | 8.293 | | | | | | | | |
| | 3 | 1.00 | 13.51 | 105.03 | 46.1 | 70.6 | 30.618 | 37.61 | 51 17 | 73.462 | | | 120.153 | 60.246 |
| | | | | | 65.58 | 22 743 | | | | | | | | |
| | 4 | 1.00 | 9 91 | 63.82 | 36.97 | 93.03 | 145.52 | 195.24 | 126.34 | 7019 | 309.32 | 178.555 | 244.522 | 164.988 |
| | 5 | 1.00 | 3.77 | 87.50 | 43.31 | 77.75 | 109.15 | 98.30 | 75.23 | 70.61 | 169.68 | 158.36 | 135.396 | 244.728 |
| | | 1.00 | 15.98 | 55.67 | 41.19 | 45.97 | 25.42 | 15.18 | 16.39 | 17.27 42.20 | 38.484 | 34.941 | 20.993 | |
| | | 1.00 | 11.15 | 66.60 | 13.14 | 37.79 | 24.4 | 21.29 | 14.54 | 23.49 | 21.67 | 43.404 | 85.106 | 73.265 |
| | 8 | 1.00 | 24.42 | 68.77 | 55.12 | 51.72 | 45.5 | 65 17 | 38.61 | 48.31 | 84 16 | 105.570 | 127.619 | 142.828 |
| Mean | | 1 00 | 11.44 | 60.10 | 9 | 51.2 | 57.08 | 55.78 | 40.84 | 38.06 | 97.54 | 87.62 | 105.77 | 101.14 |
| Sid. | | 0.00 | | 10.9 | | | 16.92 | 22.68 | 14.35 | 8.18 | 35.02 | 19.69 | 24.18 | 27.44 |
| Err. | | | | | | | | | | | | | | |

Note:
Day 9 shows individual and mean relative ROI sizes and their SEM on the day of randomization:

41-1-lee Relative ROI (photosisecond)

| Ani- | | | 2012 | 2012 | 2012 | | | | | | |
|---|---|---|---|---|---|---|---|---|---|---|---|
| mal | 2012 | 2012 May | May | May | May | 2012 May | 2012 May | 2012 May | | 2012 | 2012 Jun |
| 2C | May 9 | 11 | 14 | 17 | 21 | 24 | 28 | 31 | 2012 Jun. 4 | Jun. 9 | 11 |
| ID | Day 1 | Day 3 | Day 6 | Day 9 | Day 13 | Day 16 | Day 20 | Day 23 | Day 27 | Day 32 | Day 34 D |
| | 1.00 | 7.05 | 83.13 | 234.52 | 455.03 | 198.08 | | 435.86 | 276.3 | 21265.58 | |
| | 1.00 | 2.57 | 27.60 | 209.99 | 289.60 | 282.12 | | 550.19 | 1209.61 | | 966.69 |
| 3 | 1.00 | 5.86 | 20.92 | 51.84 | 81.32 | 140.65 | | 306.53 | 248.90 | 372.24 | |
| 4 | 1.00 | 2.91 | 17.73 | 48.05 | 95.56 | 70.71 | 112.81 | 315.24 | 246.82 | | |
| 5 | 1.00 | 8.13 | 43.82 | 138.11 | 164.26 | 565.40 | | 411.17 | 509.32 | 749.23 | 367.66 |
| 6 | 1.00 | 29 20 | 93.87 | 142.68 | 381 01 | 680 14 | | 1243.37 | | 853 54 | 652.76 |
| 7 | 1.00 | 8.13 | 46.88 | 105.68 | 163.22 | 185.17 | | 227.63 | 246.69 | 585.85 | |
| 8 | 1.00 | 19.27 | 132.74 | 134.32 | 151.18 | 282.34 | | 525.46 | 823.02 | 1414.83 | |
| Mean | 1.00 | 10.39 | 58.34 | 133.15 | 222.65 | | 300.58 | 476.63 | 560.33 | 781.75 | 367.66 |
| Std. | 0.004 | 3.251 | 14.486 | 23.494 | 48.506 | 75.2509 | 121 3788 | 127 84.38 | 145.10184 | | |
| Err, | 1.00 | 2,14 | 15.03 | 15.54 | 78.52 | 159.6 [symbol] 3 | 198.48 | 200.96 | 285.50 | 184.64 | 208.641 188.277 |
| | .00 | 13,87 | 79.43 | 107.55 | 110.24 | 162.10 | 374.88 | 224.89 | 817.71 | 1555.18 | 1518.328 334.654 |
| | 1.00 | 5,52 | 47.26 | 93.14 | 109.12 | 179.37 | 725.2$ | 943.05 | 905.55 | 1860.64 1309.003 | 1300.180 467.616 |
| | 1.00 | 39,19 | 113.88 | 225.08 | 281.13 | 440.03 | 380.70 | 344.54 | 627.20 | 1929.46 | 2966.641 |
| | .00 | 6.20 | 68.53 | 138.3 | 285 709 | 325.02 | 103278 | 1124.40 | 592.60 | 1500.42 | 534.043 |
| | 11.48 | 58.43 | 107 4 | 162.52 | 264.12 | 548.9 | 636.82 | 786.93 | 195690 | 1696.55 | 1182.22 523.83 |
| | 4 20 | 12.25 | 23.29 | 34.49 | 54.35 | 117.23 | 175.28 | 135.37 | 521.26 | 470.79 | 706.03 56 21 |
| | 1.00 | 10.89 35 | 84.79 | 25.82 | 81 39 | 727.00 | 1324.00 | 724.90 | | | |
| | 1.00 | 11.69 | 33.97 | 154.84 | 274.69 | 276.63 | 672.65 | 978.40 | 420.04 | | |
| | 1.00 | 4.51 | 53.38 | 89.13 | 72.30 | 142.35 | 354.52 | 507.65 | 161.04 | | |
| | 1.00 | 11.27 | 19.55 | 97.68 | 107.04 | 210.78 | 370.01 | 740.02 | 457.31 | | |
| | .00 | 12.99 | 43.02 | 67.98 | 19.15 | 13.82 | 97.98 | 155.39 | 246.13 | | |
| | 1.00 | 993 | 107.76 | 119.57 | 278.6 | 355.05 | 651.81 | 590.37 | 642.81 | 132.39 | |
| | .00 | 14.42 | 126.62 | 162.80 | 137.05 | 351.26 | 776.36 | 2226.72 | 2544.82 | | |
| | .00 | 3.08 | 30.39 | 75.22 | 173.33 | 282.35 | 348.43 | 472.28 | 403.87 | | |
| Mee | 1.00 | 9.85 | 56.32 | 104.0 | 135.9 | 214.2 | 499,8 | 874.3 | 700.12 | 132.39 | |
| Std. | 0.00 | 1 | 13.83 | 13.50 | 35,78 | 44.35 | 84.88 | . | 53 | | |
| Err. | | | | | | | | 229 66 | | | |

Note:
Day 9 shows individual and mean relative ROI and their SEM on the day of randomization.

4-11-Inc Relative ROI

(photos/second)

| | | 2012 | | | | 2012 | | | | | | | | |
|---|---|---|---|---|---|---|---|---|---|---|---|---|---|---|
| | | May | | | | May | 2012 | 2012 | 2012 | 2012 | 2012 | 2012 | 2012 | 2012 |
| | Ani | 9 | 2012 | 2012 | 2012 | 21 | May 24 | May 28 | May 31 | Jun. 4 | Jun. 9 | Jun. 11 | Jun. 14 | Jun. 18 |
| | mai | Day | May 11 | May 14 | May 17 | Day | Day | Day | Day | Day | Day | Day | Day | Day |
| Group | ID | 1 | Day 3 | Day 6 | Day 9 | 13 | 16 | 20 | 23 | 27 | 32 | 34 | 37 | 41 |
| AFOD | 1 | 1.00 | 8 60 | 90.88 | 108.08 | 65.00 | 127.56 | 317.21 | 1049.91 | 795.47 | 2400.18 | 4255.971 | 2019.902 | 2219.535 |
| KH | 2 | 1.00 | 13.69 | 47.87 | 186.20 | 304.58 | 518.16 | 683.84 | 986.38 | 613.03 | 242.40 | | | |
| IVI1P | 3 | 1.00 | 6.96 | 21.55 | 45.89 | 71.77 | 90.65 | 92.74 | 158.19 | 162.23 | | | | |
| OD | 4 | 1.00 | 2.13 | 14.68 | 55.67 | 108.18 | 119.14 | 266.14 | 1637.39 | | | | | |
| | 5 | 1.00 | 19.04 | 36.84 | 59.18 | 17336 | 270.65 | 288.30 | 490 65 | 1316.10 | | | | |
| | 6 | 1.00 | 18.22 | 132.18 | 198.33 | 518.13 | 599.94 | 862.62 | 1660.89 | 2273.51 | | | | |
| | 7 | 1.00 | 12.61 | 38.048 | 134.4 | 349.31 | 273.41 | 402.15 | 734.56 | 1165.41 | 1672.95 | 1858.510 | | |
| | | 1.00 | 25.46 | 92.55 | 105.7 | | 125.54 | 402.95 | 510.03 | 1268.63 | 716.23 | | | |
| Mean | | 1.00 | 13.34 | 59.32 | 96.67 | 210 8 | 265.63 | 414.49 | 903 49 | 1084.91 | 1257.94 | 3057.24 | 2019.90 | 2219.53 |
| | | | | | 111.6 | | | | | | | | | |
| Std. | | 0.00 | | 2.65 | 14 59 | 20.64 | 57.94 | 68 97 | 87.07 | 191.33 | 251.86 | 483.20 | 1198. | |
| Err, | | | | | | | | | | | | | | |
| | 1 | 1.00 | 5.34 | 112.03 | 129.10 | 110.9 | 217.65 | 271.51 | 357.68 | 734.78 | 652.75 | 1055.072 | 1506.957 | |
| | 2 | 1.00 | 7.22 | 40.72 | 159.1 | | 151.8 | 230.47 | 441.00 | 640.17 | | | | |
| | | | | | 67.93 | | | | | | | | | |
| | 3 | 1.00 | 4.22 | 26.49 | 38.50 | 81.71 | 218.99 | 248.98 | 440 55 | 222.01 | 424.13 | 202.307 | 392.014 | |
| | | 1.00 | 9.96 | 20.76 | 111 86 | | 257.74 | 888.58 | 1201.32 | | | | | |
| | | | | | 27.95 | | | | | | | | | |
| | 5 | 1.00 | 41.08 | 174.54 | 300.88 | 1071.99 | 2117.65 | 2030.73 | 6750.66 | 7402.11 | 3659.35 | 6625.988 | | |
| | 6 | 1.00 | 5.09 | 35.15 | 139.97 | | 280.91 | 340.25 | 619.85 | 348.79 | 296.14 | | | |
| | | | | | 43.83 | | | | | | | | | |
| | 7 | 1.00 | 4.68 | 16.58 | 38.64 | 56.04 | 120.04 | 158.52 | 321.38 | 286.26 | | | | |
| | 8 | 1.00 | 8.81 | 35.97 | 103.50 | | 120.33 | 249.31 | 530.70 | 897.66 | 513.39 | 15.4453 | | |
| | | | | | 84.00 | | | | | | | | | |
| Mean | | 1.00 | 10.80 | 57 78 | 229.4 | | 435.65 | 552.29 | 1332.89 | 1504 | 1101.15 | 2099.71 | 949.4 | |
| | | | | | 91.35 | | | | | | | | | |
| Std. | | 0.00 | 4.39 | 19.78 | 120.89 | | 241.23 | 226 19 | 780.19 | 987.47 | 640.19 | 151900 | 557 4 | |
| Err. | | | | | 32.12 | | | | | | | | | |

Note:
Day 9 shows individual and mean relative ROI and theft SEM on the day of randomization.

4T1-leer tumor volume {mm3)

| | | | | | | | 2012 | 2012 | | | |
|---|---|---|---|---|---|---|---|---|---|---|---|
| | 2012 May | 2012 May | 2012 May | 2012 May | 2012 May | 2012 May | Jun. 4 | Jun. 7 | 2012 Jun. | 2012 Jun. | 2012 Jun. |
| An rn | 16 | 18 | 21 | 24 | 28 | 31 | Day | Day | 11 | 14 | 18 |
| al ID | Day 8 | Day 10 | Day 13 | Day16 | Day 20 | Day 23 | 27 | 30 | Day 34 | Day 37 | Day 41 |
| | 94.06 | 148.52 | 149.87 | 264.28 | 391.14 | 666.10 | 704.38 | 962.95 | 1428.20 | 2083.21 | |
| 2 | 49 82 | 69 18 | 82.06 | 130.57 | 218.58 | 290.91 | 567.45 | 674.52 | 1022.65 | 1812.16 | |
| 3 | 79 98 | 75 26 | 95.27 | 160.48 | 239.42 | 442.24 | 677.66 | | | | |
| 4 | 107.45 | 231.83 | 251.62 | 319.99 | 602.52 | 894.39 | 1156.20 | 1632.56 | | | |
| 5 | 59.78 | 72.68 | 85.96 | 111.42 | 204.13 | 312.86 | 481.29 | 689.84 | 901.84 | | |
| 6 | 66.87 | 67.94 | 146.27 | 207.62 | 402.59 | 475.52 | 752.00 | 876.70 | 1428 20 | | |
| 7 | 88.17 | 94.63 | 136.95 | 211.74 | 408.97 | 642.66 | 954.46 | 126931 | ′164784 | | |
| 8 | 87.86 | 118.59 | 148.27 | 225.68 | 299.81 | 685 22 | 854.33 | 1194.28 | 1580.54 | | |
| Mean | 79.25 | 109.83 | 137.03 | 203.97 | 345.90 | 551 24 | 768.4 | 104238 | 1334 88 | 1947 68 | |
| Std. Err. | 29.67 | 30.03 | 50.07 | 74.13 | 122.14 | | 230.27 | 363.9197 | | 1377.22 | |
| | | | | | | | | | 67583.066 | | |
| 1 | 47.44 | 75.20 | 73.38 | 100.41 | 107.46 | 134.12 | 93.78 | 130.20 | 160.29 | 154.73 | 183.10 |
| 2 | 65.34 | 44.39 | 34.19 | 45.46 | 61.69 | 40.34 | 37 02 | 37.99 | 36.69 | 65.63 | 64.05 |
| 3 | 98.69 | 91.44 | 74.01 | 77.72 | 147.11 | 98.69 | 89.03 | 116.35 | 131.73 | 93.82 | 125.39 |
| 4 | 71.32 | 61.86 | 92.01 | 61.93 | 97.76 | 94.59 | 71.32 | 107.3 | 84.08 | 137.60 | 149.44 |
| 5 | 75.57 | 83.20 | 56.97 | 78.68 | 145.49 | 86.47 | 56.79 | 90.47 | 84.66 | 142.37 | 171.13 |
| 6 | 92 22 | 75 70 | 79.10 | 98.97 | 111.31 | 111.73 | 128.97 | 166.81 | 134.73 | 202.16 | 192.95 |
| | 103.11 | 74 76 | 88.75 | 92.67 | 124.28 | 125.84 | 101.69 | 110.06 | 126.75 | 148.88 | 168.27 |
| 8 | 85.76 | 111.65 | 77.86 | 132.40 | 94.05 | 108.34 | 100.16 | 136.35 | 128.34 | 145.86 | 169.50 |
| Mean | 79 93 | 77 28 | 72 03 | 86 03 | 111.14 | 100.01 | 84.84 | 111.95 | 110 91 | 136.38 | 152.98 |
| Sid.E | 6.60 | 7.01 | 6 58 | 97 | 9.98 | 10.19 | 10.19 | 13.31 | 13 96 | 14.49 | 14.65 |
| Cr. | | | | | | | | | | | |
| 1 | 70.68 | 101.64 | 166.51 | 279.97 | 641.22 | 804.75 | 1165.28 | | | | |
| 2 | 38.65 | 104.57 | 136.78 | 238.52 | 500.00 | 605.78 | 935.72 | | | | |
| 3 | 88.12 | 153.09 | 265.85 | 329.21 | 542.07 | 945.23 | 848.23 | | | | |
| 4 | 99.53 | 96.39 | 136.98 | 173.38 | 333.89 | 422.25 | 345.16 | 721.49 | | | |
| 5 | 65 15 | 108.77 | 102.75 | 160.88 | 253.52 | 367.82 | 570.45 | 953.79 | | | |
| 6 | 77 32 | 153.65 | 216.71 | 291.02 | 466.91 | 652.43 | 744.91 | | | | |
| | 103.13 | 120.34 | 147.85 | 224.17 | 357.51 | 519.52 | 772.79 | 852.01 | | | |
| 8 | 89.44 | 130.93 | 162.21 | 280.40 | 486.46 | 863.94 | 1210.15 | | | | |
| Mean | 79.00 | 121.17 | 166.95 | 247.19 | 450.20 | 647.72 | 824.0 | 842.4 | | | |
| | 101.8 | 7.42 | 8.01 | 18 21 | 20 84 | 44.50 | 74.08 | 87.23 | | | |

Note:
Day 8 shows individual and mean tumor sizes and their SEM on the day of randomization.

T1-leer tumor volume {mm3)

| | | | | | | | 2012 | 2012 | | | |
|---|---|---|---|---|---|---|---|---|---|---|---|
| | 2012 May | 2012 May | 2012 May | 2012 May | 2012 May | 2012 May | Jun. 4 | Jun. 7 | 2012 Jun. | 2012 Jun. | 2012 Jun. |
| Anirn | 16 | 18 | 21 | 24 | 28 | 31 | Day | Day | 11 | 14 | 18 |
| al ID | Day 8 | Day 10 | Day 13 | Day16 | Day 20 | Day 23 | 27 | 30 | Day 34 | Day 37 | Day 41 |
| | 85.44 | 96.16 | 214.61 | 390.68 | 757.32 | 436.60 | 1692.86 | 1415.40 | | | |
| 2 | 50 92 | 61 73 | 74.41 | 105.74 | 285.91 | 478.56 | 359.49 | 453.66 | | | |
| 3 | 55 33 | 73 99 | 161.55 | 190.13 | 335.16 | 482.09 | 500.95 | 654.37 | | | |
| 4 | 91.37 | 129.10 | 138.32 | 193.99 | 369.41 | 761.75 | 1299.40 | | | | |
| 5 | 67.07 | 68.01 | 144.85 | 193.75 | 297.65 | 418.71 | 603.64 | 739.64 | | | |
| 6 | 75.63 | 98.70 | 148.80 | 197.82 | 317.43 | 1062.34 | 577.84 | 610.28 | | | |
| 7 | 92.39 | 111.45 | 139.15 | 231.08 | 486.04 | 745.32 | 650.59 | 783.8 | | | |
| | 117.05 | 184.96 | 251.72 | 483.16 | 873.42 | 431.16 | | | | | |
| Mean | 79.46 | 103.01 | 159.18 | 248.29 | 465.29 | 602.00 | 812.11 | 776.1 | | | |
| St.d.Err. | 7.72 | 14.21 | 18.89 | 43.90 | 80.24 | 82.15 | 185.1 | 136.18 | | | |
| 1 | 89.27 | 102.60 | 137.52 | 192.25 | 293.61 | 436.60 | 543.08 | 668.17 | | | |
| 2 | 69.01 | 101.54 | 156.62 | 243.22 | 210.42 | 478.56 | 397.0 | 623.84 | | | |
| 3 | 47.72 | 88.29 | 154.80 | 189.42 | 275.10 | 482.09 | 807.94 | 785.60 | 1436.66 | 2022.16 | |
| 4 | 92.63 | 159.19 | 201.76 | 264.42 | 534.08 | 761.75 | 688.16 | | | | |
| 5 | 64.66 | 109.75 | 135.06 | 171.45 | 218.67 | 418 21 | 419.83 | 499.54 | 965.26 | | |
| 6 | 102.70 | 200.06 | 306.12 | 470.97 | 677.31 | 1062.34 | 1068.65 | 1068.70 | 2097.45 | | |
| 7 | 86.20 | 127.22 | 192.00 | 264.93 | 410.83 | 745.32 | 1017.62 | 1584.84 | 1783.55 | | |
| | 79.86 | 132.14 | 141.68 | 191.32 | 287.86 | 431.16 | 549.78 | 773.03 | 1004.48 | 1304.1 | |
| Mean | 79.01 | 127.60 | 178.20 | 248.50 | 363.49 | 602.00 | 686.5 | 857.6 | 1457.49 | 1663.13 | |
| Std. Err. | 6.25 | 12.98 | 20.25 | 34.31 | 58.65 | 82.15 | 91.08 | 138.40 | 2′19.52 | 359.03 | |
| 1 | 54.62 | 90.36 | 115.03 | 152.81 | 243.32 | 382 69 | 517.45 | | | | |
| 2 | 91.81 | 105.45 | 112.06 | 157.00 | 222.99 | 374.34 | 442.28 | 684.1 | | | |
| 3 | 55.82 | 66.57 | 96.65 | 115.62 | | | | | | | |
| 4 | 81.47 | 118.18 | 160.72 | 233.40 | 375.67 | 853.53 | 1028.22 | 1056.02 | 1684.53 | 1697.99 | |
| 5 | 109.61 | 148.72 | 231.72 | 185.33 | 364.62 | 613.45 | 733.15 | | | | |
| 6 | 73.04 | 95.68 | 110.36 | 245.62 | 238.13 | 272.40 | 408.16 | 384.97 | | | |
| 7 | 98.07 | 128.34 | 164.49 | 236.79 | 601.59 | 953 12 | 1256.16 | | | | |
| 8 | 65.51 | 131.63 | 139.20 | 191.93 | 419.47 | 937.66 | 1005.99 | | | | |
| Mean | 78.74 | 110.62 | 141.28 | 189.81 | 352.26 | 770.20 | 708.3 | 1684;3 | 169799 | | |
| | | | | | | | 626 74 | | | | |
| | 125.56 | 194.09 | 7.10 | 9.32 | 15 58 | 16 47 | 50.94 | 109.50 | #ONV0! | #001103 | |

Note:
Day 8 shows individual and mean tumor sizes and their SEM on the day of randomization.

41-1-11. lc tumor volume (arn3)

| | 2012 May | 2012 May | 2012 May | 2012 May | 2012 May | 2012 May | 2012 | 2012 | 2012 Jun. | 2012 Jun. | 2012 Jun. |
|---|---|---|---|---|---|---|---|---|---|---|---|
| Animal | 16 | 18 | 21 | 24 | 28 | 31 | Jun. 4 | Jun. 7 | 11 | 14 | 18 |
| ID | Day 8 | Day 10 | Day 13 | Day16 | Day 20 | Day 23 | Day 27 | Day 30 | Day 34 | Day 37 | Day 41 |
| 1 | 85.67 | 121.25 | 134.90 | 189.80 | 274.60 | 352 71 | 587.58 | 687.49 | 1074.62 | 1703.09 | 1405.32 |
| 2 | 38.03 | 50.69 | 73.72 | 132.97 | 189.33 | 283.84 | 438.56 | 555.07 | 803.03 | 1006.66 | |
| 3 | 66.10 | 104.29 | 112.50 | 193.20 | 298.69 | 339.44 | 423.00 | 636.13 | 1123.49 | | |
| 4 | 97.30 | 108.71 | 167.92 | 271.07 | 448.46 | 603.40 | 776.75 | 1111.93 | 1491.19 | | |
| 5 | 58 53 | 85.49 | 104.26 | 174.74 | 201.29 | 342.92 | 493.89 | 607.62 | 1062.24 | 1504.87 | |
| 6 | 129.44 | 99.09 | 244.94 | 209.93 | 309.84 | 397.43 | 655.51 | 1041.71 | 1050.49 | 1880.77 | 2122.77 |
| | 78.52 | 106.50 | 128.01 | 157.84 | 207.89 | 328.74 | 616.15 | 797.94 | 765.60 | | |
| 8 | 90.54 | 103.68 | 130.30 | 157.12 | 220.84 | 464.76 | 525.82 | 378.23 | 442.39 | | |
| rr, | 9.73 | | 18.13 | 14.91 | 30.41 | 35.95 | 42.02 | 87.37 | 109.28 | 188.72 | 358.72 |
| 1 | 64.81 | 125.84 | 217.66 | 296.50 | 324.40 | 520.83 | 1043.53 | 1026.79 | | | |
| 2 | 70.12 | 116.78 | 168.37 | 190.65 | 294.56 | 299.06 | 450.37 | 591.80 | | | |
| 3 | 52 51 | 79 80 | 87.22 | 174.60 | 421.15 | 773.26 | 875.45 | | | | |
| 4 | 102.90 | 103.73 | 152.28 | 212.99 | 294.02 | 352.00 | 344.68 | | | | |
| 5 | 72.98 | 131.41 | 176.23 | 321.87 | 259.88 | 211.03 | 387.83 | 489.54 | | | |
| 6 | 87.79 | 106.50 | 111.85 | 189.54 | 240.60 | 316.31 | 451.18 | 625.43 | | | |
| 7 | 94.31 | 152.02 | 252.37 | 359.40 | 561.72 | 686.31 | 745.77 | 691.84 | | | |
| 8 | 90.41 | 117.12 | 114.57 | 225.74 | 574.43 | 827.98 | 1082.50 | 1213.19 | | | |
| Mean | 79.48 | 116.65 | 160.07 | 246.41 | 371.34 | 498.35 | 672.66 | 773.10 | | | |
| Std. Err. | 6.02 | 7.54 | 19.79 | 24.65 | 46.97 | 84.19 | 106.83 | 115.43 | | | |
| 1 | 57.29 | 87.98 | 107.10 | 201.40 | 194.49 | 297.17 | 441.28 | 609.09 | 902.44 | 1395.06 | 1477.22 |
| 2 | 77.40 | 95.98 | 114.74 | 204.71 | 256.07 | 278.82 | 330.39 | 465.16 | | | |
| 3 | 46.53 | ‘108.84 | 102.24 | 185.95 | 296.26 | 626.97 | 666.49 | | | | |
| 4 | 85.98 | 121.93 | 148.80 | 307.80 | 586.48 | 850.37 | | | | | |
| 5 | 70 34 | 101.03 | 111.66 | 170.63 | 247.20 | 407.80 | 510.44 | | | | |
| 6 | 95.60 | 108.22 | 113.64 | 228.91 | 300.02 | 493.32 | 610.80 | 618.20 | | | |
| 7 | 112.01 | 123.93 | 147.77 | 225.34 | 315.49 | 342.33 | 546.38 | 699.19 | 1014.43 | | |
| 8 | 89.99 | 120.02 | 125.47 | 174.62 | 259.91 | 325.49 | 440.22 | 559.10 | | | |
| Mean | 79.39 | 108.49 | 121.43 | 212.42 | 306.99 | 452.78 | 506.5 | 590.1 | 958.43 | 1395 06 | 1477.2 |
| Std. Err, | 7.43 | 4.60 | 6.32 | 15.58 | 42.13 | 70.07 | 43.02 | 38.49 | 55.99 | | |

Note:
Day 8 shows individual and mean tumor sizes and their SEM on the day of randomization.

4T1-leer tumor volume (mm3)

| | 2012 May | 2012 May | 2012 May | 2012 May | 2012 May | 2012 May | 2012 | 2012 | 2012 Jun. | 2012 Jun. | 2012 Jun. |
|---|---|---|---|---|---|---|---|---|---|---|---|
| Anirnal | 16 | 18 | 21 | 24 | 28 | 31 | Jun. 4 | Jun. 7 | 11 | 14 | 18 |
| ID | Day 8 | Day 10 | Day 13 | Day16 | Day 20 | Day 23 | Day 27 | Day 30 | Day 34 | Day 37 | Day 41 |
| 1 | 52.18 | 78.55 | 90.74 | 160.68 | 156.58 | 173.80 | 266.84 | 354.94 | 423.54 | 655.31 | |
| 2 | 65 15 | 92 83 | 112.49 | 223.63 | 220.17 | 405.91 | 511.79 | | | | |
| 3 | 105.02 | 167.48 | 179.33 | 194.44 | 495.33 | 774.57 | 1329.96 | 1147.06 | 1871.70 | 1899.26 | |
| 4 | 66.99 | 79.18 | 123.42 | 208.09 | 253.02139 | 364.76 | 512.4 | | | | |
| | 82.62 | 91.23 | 116.35 | 203.29 | 590.36 | 663 61 | 1027.12 | 1061.84 | 1222.18 | | |
| 6 | 92.37 | 83.34 | 95.94 | 201.32 | 607.67 | 717.20 | 586.31 | 608.77 | | | |
| 7 | 73.42 | 90.51 | 131.17 | 214.25 | 358.34 | 551.89 | 583.5 | | | | |
| | 100.53 | 127.98 | 189.03 | 261.30 | 364.77 | 515.14 | 706.55 | 913.28 | 1116.28 | | |
| Mean | 79.79 | 101.39 | 129.81 | 208.33 | 330.78 | 520.86 | 690.5 | 817.13 | 1158.48 | 1277.2 | |
| St.d. Err. | 6.57 | 10.95 | 12.79 | 10.01 | 60.01 | 71.00 | 118.6 | 147.4 | 296.46 | 621.97 | |

Note:
Day 8 shows individual and mean umor sizes and their SEM on the day of randomization.

41-1-lec orthotopic Body weight (g)

| | 2012 May | 2012 May | 2012 May | 2012 May | 2012 May | 2012 May | 2012 | 2012 | 2012 Jun. | 2012 Jun. | 2012 Jun. |
|---|---|---|---|---|---|---|---|---|---|---|---|
| | 16 | 18 | 21 | 24 | 28 | 31 | Jun. 4 | Jun. 7 | 11 | 14 | 18 |
| NO. | Day 8 | Day 10 | Day 13 | Day16 | Day 20 | Day 23 | Day 27 | Day 30 | Day 34 | Day 37 | Day 41 |
| 1 | 23 64 | 22 47 | 23.76 | 23.09 | 23.62 | 24.03 | 23.13 | 23.09 | 19 72 | 21.30 | |
| | 19.93 | 19.43 | 20.20 | 20.61 | 20.76 | 21.17 | 20 90 | 19.65 | 19.17 | 19.18 | |
| 3 | 20.80 | 20.25 | 21.18 | 21.43 | 21.56 | 21.80 | 19.66 | | | | |
| 4 | 21.22 | 20.89 | 22.10 | 22.13 | 22.85 | 22.46 | 22.05 | 20.84 | | | |
| 5 | 20.95 | 22.00 | 21.15 | 21.78 | 22.30 | 22.05 | 22.89 | 21.25 | 20.47 | | |
| 6 | 22.58 | 20.28 | 22.89 | 23.59 | 23.76 | 24.13 | 24.50 | 22.71 | 19.72 | | |
| 7 | 20.42 | 20.22 | 20.48 | 20.95 | 20.44 | 20.27 | 20.19 | 20.52 | 22.56 | | |
| 8 | 20.98 | 24.59 | 25.14 | 25.47 | 25.75 | 25.34 | 22.83 | 22.16 | 19.94 | | |
| Mean | 21.32 | 21.27 | 22.11 | 22.38 | 22.63 | 22.66 | 22.02 | 21.46 | 20.26 | 20.49 | |
| Std. Err. | 7.41 | 7.28 | 7.65 | 7.76 | 7.98 | 7.87 | 7.10 | 8.03 | 8.10 | 14.49 | 24.46 |
| 1 | 23.17 | 21.39 | 22.73 | 23.14 | 21.08 | 24.56 | 24.02 | 24.07 | 23.69 | 24.69 | |
| 2 | 21.11 | 19.86 | 21.03 | 21.20 | 23.13 | 21.46 | 21.72 | 22.19 | 21.44 | 22.53 | 23.17 |
| 3 | 22.41 | 20.16 | 21.76 | 22.43 | 22.75 | 22.56 | 23.13 | 22.84 | 22.54 | 24.43 | 21.47 |
| 4 | 22.47 | 20.89 | 21.96 | 21.86 | 21.69 | 21.69 | 21.78 | 22.58 | 21.70 | 22.86 | 22.54 |
| 5 | 22 33 | 20 93 | 21.63 | 21.76 | 21.87 | 21.71 | 22.27 | 22.07 | 21 68 | 22.63 | 22.62 |
| | 19.21 | 15.57 | 17.85 | 19.68 | 20.28 | 20.19 | 18 60 | 19.32 | 18.98 | 20.67 | 20.8 |
| 7 | 23.08 | 21.94 | 23.21 | 23.69 | 22.34 | 24.93 | 25.64 | 25.44 | 24.96 | 26.92 | 26.8 |
| 8 | 22.00 | 20.24 | 21.86 | 22.09 | 24.36 | 22.93 | 23.27 | 23.21 | 21.85 | 23.93 | 23.1 |
| Mean | 21.97 | 20.12 | 21.50 | 21.98 | 22.19 | 22.50 | 22 55 | 22.72 | 22.11 | 23.58 | 23.12 |
| Std. Err. | 0.46 | 0.69 | 0.57 | 0.43 | 0.45 | 0.57 | 0.73 | 0.62 | 0.62 | 0.66 | 0.66 |
| 1 | 20.45 | 19.71 | 20.28 | 20.51 | 20.88 | 19.58 | 18 87 | | | | |
| 2 | 24.26 | 22.93 | 23.30 | 23.91 | 24.24 | 23.15 | 22.50 | | | | |
| | 21.30 | 21.09 | 21.82 | 22.51 | 22.49 | 23.02 | 20.38 | | | | |
| 4 | 20.01 | 19.20 | 19.80 | 19.95 | 20.23 | 20.16 | 20.31 | 19.62 | | | |
| | 20.67 | 20.06 | 21.02 | 21.97 | 22.31 | 22.29 | 22.73 | 24.59 | | | |
| 6 | 20 44 | 20 08 | 20.36 | 20.54 | 20.02 | 20.64 | 19.65 | | | | |
| 7 | 22.53 | 22.02 | 23.04 | 23.72 | 24.17 | 24.29 | 25 07 | 23.89 | | | |
| | 20.62 | 20.37 | 21.09 | 21.95 | 22.70 | 23.27 | 22.22 | | | | |
| Mean | 21.29 | 20.68 | 21.34 | 21.88 | 22.13 | 22.05 | 21.47 | 22.70 | | | |
| Std. E | 0.50 | 0.44 | 0.45 | 0.52 | 0.58 | 0.60 | 0.72 | 1.55 | | | |

Note:
Day 8 shows individual and mean body weight and their SEM on the day of randomization.

41-1-luc orthotopic Body weight (g)

| | 2012 | 2012 | 2012 | 2012 | 2012 | 2012 | 2012 | 2012 | 2012 | 2012 | 2012 |
|---|---|---|---|---|---|---|---|---|---|---|---|
| | May 16 | May 18 | May 21 | May 24 | May 28 | May 31 | Jun. 4 | Jun. 7 | Jun. 11 | Jun. 14 | Jun. 18 |
| NO. | Day 8 | Day 10 | Day 13 | Day 16 | Day 20 | Day 23 | Day 27 | Day 30 | Day 34 | Day 37 | Day 41 |
| | 20 41 | 20 09 | 20.96 | 22.09 | 21.68 | 22.55 | 21.62 | 22.44 | | | |
| | 22.04 | 20.52 | 21.93 | 22.51 | 21.76 | 22.23 | 22 82 | 23.23 | | | |
| 3 | 19.83 | 19.58 | 20.75 | 20.91 | 21.10 | 20.64 | 21.44 | 21.45 | | | |
| 4 | 23.98 | 21.56 | 22.82 | 23.17 | 23.55 | 23.68 | 23.57 | | | | |
| 5 | 22.04 | 21.26 | 21.08 | 22.23 | 22.57 | 23.81 | 22.40 | 21.09 | | | |
| 6 | 21.60 | 20.89 | 21.57 | 22.25 | 22.77 | 23.03 | 20.98 | 21.41 | | | |
| 7 | 21.33 | 20.50 | 21.67 | 22.02 | 21.58 | 21.82 | 22.63 | 22.56 | | | |
| 8 | 23.19 | 22.44 | 23.16 | 93.59 | 23.55 | 21.89 | | | | | |
| Mean | 21.80 | 20.86 | 21.74 | 22.35 | 22.32 | 22.46 | 22.21 | 22.03 | | | |
| Std. Err. | 0.48 | 0.32 | 0.31 | 0.28 | 0.33 | 0.37 | 0.34 | 0.34 | | | |
| 1 | 24.04 | 23.45 | 24.15 | 24.04 | 24.52 | 24.33 | 23.49 | 22.45 | | | |
| 2 | 21.14 | 20.75 | 21.51 | 22.07 | 22.05 | 21.12 | 20.33 | 20.77 | | | |
| 3 | 22.07 | 22.11 | 23.09 | 23.40 | 23.30 | 24.24 | 24.69 | 25.39 | 24.39 | 23.78 | |
| 4 | 22.00 | 20.85 | 21.84 | 22.15 | 22.23 | 22.60 | 23.36 | | | | |
| 5 | 22 89 | 22 08 | 22.67 | 22.89 | 23.01 | 23.34 | 23.59 | 23.26 | 22 00 | | |
| | 21.72 | 21.21 | 21.46 | 21.77 | 21.83 | 22.34 | 23 55 | 23.25 | 21.79 | | |
| -r | 23.99 | 22.69 | 24.13 | 24.51 | 25.22 | 24.68 | 25.18 | 25.88 | 24.82 | | |
| 8 | 21.74 | 21.39 | 22.25 | 22.9 | 22.85 | 22.5 | 22.65 | 23.10 | 21.24 | 20.17 | |
| Mean | 22.45 | 21.82 | 22.64 | 22.97 | 23.13 | 23.14 | 23 36 | 23.44 | 22.95 | 21.98 | |
| Std. Err. | 0.38 | 0.33 | 0.38 | 0.34 | 0.42 | 0.43 | 0.52 | 0.66 | 0.79 | 1.81 | |
| 1 | 22.06 | 20.26 | 20.12 | 19.55 | 20.94 | 22.07 | 22 76 | | | | |
| 2 | 20.70 | 20.16 | 19.74 | 21.58 | 22.38 | 23.04 | 22.84 | 23.24 | | | |
| 3 | 19.98 | 19.90 | 19.36 | 20.15 | | | | | | | |
| 4 | 21.89 | 22.86 | 23.08 | 20.8 | 25.06 | 24.71 | 23.90 | 25.70 | 25.13 | 73.93 | |
| | 23.61 | 23.03 | 22.22 | 25.1 | 25.07 | 25.83 | 24.38 | | | | |
| 6 | 21 42 | 20 34 | 20.36 | 24.2 | 21.47 | 21.89 | 22.53 | 21.14 | | | |
| 7 | 24.50 | 24.07 | 20.93 | 22.12 | 24.53 | 25.54 | 26 55 | | | | |
| | 21.11 | 20.34 | 21.13 | 22.69 | 22.63 | 23.13 | 23.48 | | | | |
| Mean | 21.91 | 71.37 | 20.93 | 22.02 | 23.16 | 23.74 | 23.78 | 23.36 | 25.13 | 23.93 | |
| Std. E | 0.53 | 0.59 | 0.44 | 0.68 | 0.65 | 0.61 | 0.53 | 1.32 | | | |

Note:
Day 8 shows individual and mean body weight and theft SEM on the day of randomization.

411-113c orthotopic Body weight (g)

| | 2012 | 2012 | 2012 | 2012 | 2012 | 2012 | 2012 | 2012 | 2012 | 2012 | 2012 |
|---|---|---|---|---|---|---|---|---|---|---|---|
| Animal | May 16 | May 18 | May 21 | May 24 | May 28 | May 31 | Jun. 4 | Jun. 7 | Jun. 11 | Jun. 14 | Jun. 18 |
| ID | Day 8 | Day 10 | Day 13 | Day 16 | Day 20 | Day 23 | Day 27 | Day 30 | Day 34 | Day 37 | Day 41 |
| 1 | 21.28 | 20.50 | 21.41 | 21.06 | 21.02 | 21.15 | 20.53 | 20.76 | 19.36 | 20.29 | 20.12 |
| 2 | 21.00 | 20.02 | 21.47 | 21.15 | 21.5 | 21.32 | 21.32 | 21.59 | 21.19 | 19.46 | |
| 3 | 22.01 | 21.58 | 21.99 | 21.95 | 21.75 | 22.18 | 22.79 | 22.61 | 20.29 | | |
| 4 | 21 27 | 19 68 | 20.77 | 21.44 | 20.96 | 20.77 | 20.41 | 20.80 | 20 04 | | |
| | 19.73 | 19.24 | 19.86 | 20.59 | 20.52 | 20.73 | 20 96 | 20.94 | 21.30 | 20.18 | |
| | 21.69 | 21.74 | 21.33 | 22.27 | 22.76 | 23.91 | 24.39 | 24.45 | 22.79 | 24.01 | 23.18 |
| 8 | 21.53 | 20.51 | 22.29 | 22.14 | 22.37 | 22.36 | 23.56 | 20.54 | 19.30 | | |
| | y | | | | | | | | | | |
| Mean | 21.44 | 2′0.74 | 2 | 21.82 | 21.95 | 22.11 | 22.33 | 21.86 | 20.67 | 2 | 21.65 |
| Std. E | 0.33 | 0.41 | 038 | 0 40 | 0.48 | 0.49 | 0.62 | 0.50 | 0.41 | 1 02 | 1 53 |
| Cr. | | | | | | | | | | | |
| 1 | 21.20 | 20.84 | 21.25 | 22.55 | 22.2 | 21.38 | 19.59 | 22.15 | | | |
| 2 | 19.73 | 19.16 | 20.04 | 20.84 | 20.78 | 20.32 | 20.41 | 19.33 | | | |
| 3 | 21.72 | 20.85 | 21.46 | 21.27 | 21.57 | 22.19 | 18 07 | | | | |
| 4 | 22 13 | 21 70 | 22.66 | 23.2 | 23.27 | 23.99 | 19.77 | | | | |
| | 22.57 | 21.57 | 22.50 | 20.47 | 21.27 | 22.3 | 23.00 | 22.27 | | | |
| | 21.01 | 20.92 | 22.19 | 22.44 | 22.83 | 23.67 | 23.65 | 24.81 | | | |
| 7 | 22.52 | 21.04 | 21.57 | 21.55 | 23.71 | 22.98 | 21.14 | 21.64 | | | |
| | 24.79 | 19.99 | 21.02 | 21.65 | 22.59 | 22.36 | 21.94 | 22.52 | | | |
| Mean | 21.96 | 20.76 | 21.59 | 21.75 | 22.28 | 22.40 | 20 95 | 22.12 | | | |
| Std. E | 0.2 | 0.29 | 0 30 | 0 33 | 0.36 | 0.42 | 0.66 | 0.72 | | | |
| Cr. | | | | | | | | | | | |
| 1 | 23.21 | 22.05 | 23.24 | 23.13 | 23.58 | 24.39 | 24.24 | 24.20 | 25.18 | 24.55 | 22.56 |
| 2 | 21.23 | 20.79 | 21.65 | 21.70 | 21.31 | 20.49 | 18.73 | 19.37 | | | |
| 3 | 23.23 | 22.72 | 23.54 | 23.46 | 23.58 | 22.92 | 20.88 | | | | |
| 4 | 21.61 | 20.50 | 21.88 | 21.89 | 21.98 | 21.71 | | | | | |
| | 20.47 | 19.86 | 20.61 | 21.05 | 21.06 | 21.62 | 19 59 | | | | |
| 6 | 20 83 | 20 74 | 20.68 | 21.83 | 22.37 | 22.75 | 23.22 | 21.49 | | | |
| 7 | 20.78 | 20.92 | 21.57 | 23.00 | 22.58 | 22.47 | 22.94 | 23.15 | 21.71 | | |
| | 22.40 | 21.30 | 22.20 | 21.65 | 21.9 | 22.23 | 23.11 | 21.98 | | | |
| Mean | 21.72 | 21.11 | 21.92 | 22.21 | 22.30 | 22.32 | 21 82 | 22.04 | 23.45 | 24.55 | 22.56 |
| Std. E | 0.39 | 0.32 | 0 38 | 0 30 | 0 33 | 0 40 | 0.79 | 0.82 | 1.73 | | |

Note:
Day 8 shows individual and mean body weight and their SEM on the day of randomization.
Note:
Day 8 shows individual and mean body weight and their SEM on the day of randomization.

Exhibit 3: Daily testin articles record.

| Group | Vehicie | Gerncitabine | Afod raas 1 | Mod raas 2 | Mod raas 3 |
|---|---|---|---|---|---|
| 2012 May 16 | Day 1 - | Gerncitabin | Afod raas | Afod raas 2(15%) | Afod raas 3(20%) |
| | + | 0 | 1(10%)iv0.2mi | iv0.2m1 | iv0.2m1 |
| | | | + | | |
| 2012 May 17 | Day 2 .. | | Afod raas 1(10%) | Afod raas 2(15%) | Afod raas 3(20%) |
| | | | iv0.2m1 | iv0.2mi | iy0.2m1 |
| 2012 May 18 | Gaya - | | Afod raas 1(10%) | Afod raas 2(15%) | Afod raas 3(20%) |
| | | | ip0.4rni | if″0.4mi | ip0.4m1 |
| 2012 May 19 | Day 4 - | | Afod raas 1(10%) | Afod raas 2(15%) | Afod raas 3(20%) |
| | | | iv0.2m1 | iv0.2rni | iv0.2rni |
| 2012-5- | Day 5 .. | Gemcitabine | Afod raas 1(10%) | Afod raas 2(15%) | Afod raas 3(20%) |
| -)f- | | | iv0.2m1 | iv0.2rni | iv0.2rni |
| -_” | | | | | |
| 2012 May 21 | Day 6 - | | Afod raas 1(10%) | Afod raas 2(15%) | Afod raas |
| | | | ip0.4m1 | if-‘0.4mi | 3(20%)1130.4rni |
| 2012 May 22 | Day 7 - | | Atod raas 1(10%) | Afod raas 2(15%) | Afod raas 3(20%) |
| | + | | iv0.2m1 | iv0.2m1 | iv0.2m1 |
| 2012 May 23 | Day 8 .. | Gemcitabin | IMMAt.‘014gORMME | Afod raas 2(25%) | Afod raas |
| | | | iMMMMMMMMMMM | iv0.2m1 | 3(20%)iy0.2m11 |
| | | | .................................................... | | |
| | | | e hgggEMROCEgggg | | |
| 2012 May 24 | Day 9 - | | Afod raas 8(25%) | Afod raas 2(25%) | Afod raas |
| | | | ip0.4rni | if:‘0.4m( | 3(20%)ip0.4m1 |
| 2012 May 25 | Day - | | Afod raas 8(25%) | Atod raas 2(25%) | AG£13′: s |
| | 10 | | ip0.4m1 | iP0.4mi | 3f3 ME £14f: 1 |
| 2012 May 26 | Day .. | | Afod raas 8(25%) | ,,,,,,,,,,,,,,,,,,,,,,,,,,,,,,,,,,,,,, | ,,,,,,,,,,,,,,,,,,,,,,,,,,,,, |
| | 11 | | ip0.4rril | ,,,,,,,,,,,,,_{—} | ,,,,,,,,,,,,,,,,,,,,,,, |
| | | | | Eggggggggggggggn | Afod raas |
| | | | | --A-foow2m.0004te | 3(40%)ip0.4mi |
| 2012 May 27 | Day - | Gernoitabina | Afod raas 8(25%) | Afod raas 2(29%)iv0 | Mod raas |
| | 12 | | iy0.2ra1 | 2rni | 3(40%)iv0 2rn1 |
| 2012 May 28 | Day - | | Atod raas 8(25%) | Afod raas | Afod raas |
| | 13 | | ip0.4m1 | 2(29′34)0.4mi | 3(40%)1130.4rni |
| 2012-5- | Day - | ,\,.·x\-,k.″4 | :,.* .-, | ,,.x\-:., x ' §, | drAfo s |
| 90 | 14 | | | | 3(40%)00.4m1 |
| -, | | | | | |
| 2012 May 30 | Day - | Gemoitabin | Afodraas 8(2%) | Afod raas 2)..21 | Afod raas |
| | 15 | 0 | iv0.2m1 | | 3(40′30v0.2m1 |
| 2012 May 31 | Day - | | Afod raas 8(2%) | Afod raas | raas |
| | 16 | | ip0.4rni | 2(%)ip0.4m1Afod | 3(40%)00.4rni |
| | + | | + | | |
| 2012 Jun. 1 | Day .. | | Afod raas 8(2%) | Afo, raas 2(%)00.4w | Afod raas |
| | 17 | | ip0.4i | | 3(40%)00.4rni |
| 2012 Jun. 2 | Day - | | Afod raas 8(2%) | Afod raas 2(%)00.46 | Afod raas |
| | 18 | | ip0.4l | | 3(40%)ip0.4nil |

Note:
Day las the first dosing day.

| Group | Vehicle | Gerneitabirta | Afod raas 1 | Afod raas 2 | Afod raas 3 |
|---|---|---|---|---|---|
| 2012 Jun. 3 | Day-19 | Gerneitabine | Afod raas 8(? %) | Afod raas 2(%) | Afod raas 3(40° 1) |
| | | | ip0.4 m1 | 00.4 m1 | 00.4 ml |
| 2012 Jun. 4 | Day-20 | | Afod raas 8(?%) | Afod raas 2(%) | Afod rads 3(40%) |
| | | | ip0.4 rn1 | 00.4 rn1 | ip0.4 rni |
| 2012 Jun. 5 | Day-21 | | Afod raas 8(? %) | Afod raas 2(%) | Afod raas 3(40%) |
| | | | ip0.4 rni | 00.4 rni | 00.4 m1 |
| 2012 Jun. 6 | Day-22 | Genicita(7in0 | Afod raas 8(25%) | W %.1 − 0 W−−gg9.−, −, %) | iiiOARIMPA0ii:iAf0VOi:i0g |
| | | | ip0.6 m1 | 10 fMiriii | |
| 2012 Jun. 7 | Day-23 | | Afod raas 8(25%) | Afod raas 2(29%) | Afod raas 3(20%) |
| | | | ipB1D1.0 rol | ipBID1 m1 | ipSID1 ni1 |
| 2012 Jun. 8 | Day-24 | | Afod raas 8(25%) | Afod raas 2(29%) | Afod raas 3(20%) |
| | | | ipl.0 rn1 | iplml | iplrol |
| 2012 Jun. 9 | Day-25 | | | | |
| 2012 Jun. 10 | Day 96 | Geractabine | | | |
| 2012 Jun. 11 | Day 27 | | | | |
| 2012 Jun. 12 | Day 23 | | | | |
| 2012 Jun. 13 | Day-29 | Gerncitabin0 | | −−−−Afod | raas 3(20%) |
| | | | | | 00.4 m1 |
| 2012 Jun. 14 | Day-30 | | | | Afod raas 3(20%) |
| | | | | | 00.4 m1 |
| 2012 Jun. 15 | Day-31 | | | | Afod raas 3(20°, 1) |
| | | | | | 00.8 ml |
| 2012 Jun. 16 | Day-32 | | | | |
| 2012 Jun. 17 | Day-33 | Gemcitabine | | | Afod raas 3(20%) |
| | | | | | 00.8 m1 |
| 2012 Jun. 18 | Day-34 | | | | |
| 2012 Jun. 19 | Day 35 | | | | |
| 2012 Jun. 20 | Day 36 | Gernoitabine | | | |

Note:
Day las the first dosing day.

| | Group | Afod raas 4 | Afod raas 5 | Afod raas 6 | Mod kh | Moe kh |
|---|---|---|---|---|---|---|
| 2012 May 16 | Day 1 | Afod raas 4(10%) | Afod raas 5(5%) | Afod raas 6(5%) | Afod kh(20%) | Afcc kh(18%) |
| | | iv0.2 m1 | iv0.2 m1 | iv0.2 m1 | iv0.2 m1 | iv0.2 m1 |
| 2012 May 17 | Day 2 | Afod raas 4(10%) | Afod raas 5(5%) | Afod raas 6(5%) | Afod kh(20%) | Afcc kh(18%) |
| | | iv0.2 m1 | iv0.2 m1 | iv0.2 m1 | iv0.2 m1 | iv0.2 m1 |
| 2012 May 18 | Day 3 | Afod raas 4(10%) | Afod raas 5(5%) | Mod raas 6(5%) | Mod kh(20%) | Afcc kh(18%) |
| | | ip0.4 m1 | ip0.4 m1 | ip0.4 m1 | ip0.4 m1 | ip0.4 m1 |
| 2012 May 19 | Day 4 | Afod raas 4(10%) | Afod raas 5(5%) | Afod raas 6(5%) | Afod kh(20%) | Afcc kh(18%) |
| | | iv0.2 m1 | iv0.2 m1 | iv0.2 m1 | iv0.2 m1 | iv0.2 m1 |
| 2012 May 20 | Day 5 | Afod raas 4(10%) | Afod raas 5(5%) | Afod raas 6(5%) | Afod kh(20%) | Afcc kh(18%) |
| | | iv0.2 m1 | iv0.2 m1 | iv0.2 m1+ | iv0.2 m1 | iv0.2 m1 |
| 2012 May 21 | Day 6 | Afod raas | Mod raas 5(5%) | Afod raas 6(5%) | Mod kh(20%) | Afcc kh(18%) |
| | | 4(10%)ip0.4 m1 | 00.4 rni | 00.4 m1 | 1p0.4 m1 | ip0.4 m1 |
| 2012 May 22 | Day 7 | Afod raas 4(10%) | Afod raas 5(5%) | Afod raas 6(5%) | Afod kh(20%) | Afcc kh(18%) |
| | | iv0.2 m1 | iv0.2 m1 | iv0.2 m1 | iv0.2 m1 | iv0.2 m1 |
| 2012 May 23 | Day 8 | Afod raas 4(10%) | Afod raas 5(5%) | Afod raas 6(5c %) | Afod kh(20%) | Afcc kh(18%) |
| | | iv0.2 m11 | iv0.2 m1 | iv0.2 m1 | iv0.2 m1 | iv0.2 m1 |
| 2012 May 24 | Day 9 | Afod raas 4(10′34) | Afod raas 5(5%) | gg0* − Atb11, f0fa.MEN Afod | | Afcc kh(18%) |
| | | 10.4 m1 | ip0.4 m1 | MMMMMEME1 iiiiniNW*, | | 00.4 m1 |
| | | | | ?iiMi.4.Mignil kh(20%)ip0.4 m1 | | |
| 2012 May 25 | Day 10 | Mod raas 4(10%) | Afod raas 5(5%) | Afod raas 6(8%) | Afod kh(20%) | Afcc kh(18%) |
| | | 00.4 m1 | 00.4 m11 | ip0.4 m1 | ip0.4 m1 | 00.4 m1 |
| 2012 May 26 | Day 11 | Afod raas 4(10%) | Afod raas 5(5%) | Afod raas 6(8%) | Afod kh(20%) | Afcc kh(18%) |
| | | ip0.4 m1 | ip0.4 m1 | ip0.4 mi+ | ip0.4 m1 | ip0.4 m1 |
| 2012 May 27 | Day 12 | Afod raas 4(10%) | Mod raas 5(5%) | Afod raas 6(8%) | Mod kh(20%) | Afcc kh(18%) |
| | | iv0.2 m1 | 1v0.2 m1 | iv0.2 m1 | iv0.2 m1 | iv0.2 m1 |
| 2012 May 28 | Day 13 | Afod raas 4(10%) | Afod raas 5(59/0) | Afod raas 6(8%) | Afod kh(20%) | Afcc kh(18%) |
| | | ip0.4 m1 | 00.4 mi | 00.4 m1 | 1p0.4 m1 | ip0.4 m1 |
| 2012 May 29 | Day 14 | Afod raas 4(10%) | Mod raas 5(5%) | Afod raas 6(8%) | Mod kh(20%) | Afcc kh(18%) |
| | | 1p0.4 m1 | 00.4 m1 | 00.4 rn1 | ip0.4 m1 | ip0.4 m1 |
| 2012 May 30 | Day 15 | Afod raas 4(10%) | Mod raas 5(5%) | ..−*, AfcAraas | 1 > 1111 > 1111 > E | Afcc kh(18%) |
| | | iv0.2 m1 | iv0.2 m1 | Afod li*V*MOW | kh(20%)iv0.2 m1 | iv0.2 mi |
| 2012 May 31 | Day 16 | Afod raas 4(10%) | Afod raas 5(5%) | Afod raas 6(25%) | Afod kh(20%) | Afcc kh(18%) |
| | | 00.4 m1 | 00.4 rril | 00.4 m1 | ip0.4 m1 | 00.4 m1 |
| 2012 Jun. 1 | Day 17 | Mod raas 4(10′34) | Afod raas 5(5%) | Mod raas 6(25%) | Afod kh(20%) | Afcc kh(18%) |
| | | 430.4 rd | 00.4 m1 | 00.4 rni | ip0.4 m1 | 00.4 m1 |
| 2012 Jun. 2 | Day 18 | Afod raas 4(10%) | Afod raas 5(5°1) | Mod raas 6(25%) | Afod kh(20%) | Afcc kh(18%) |
| | | 00.4 rni | 00.4 ml | 00.4 mi | ip0.4 m1 | 00.4 m1 |

Note:
Day las the first dosing day.

| | | *k oup | | | | |
|---|---|---|---|---|---|---|
| Date | | Afod raas 4 | Afod raas 5 | Afod raas 6 | Afod kh | Afcc kh |
| 2012 Jun. 3 | Day 19 | Afod raas 4(10%) | Afod raas 5(5%) | Afod raas 6(25%) | Afod k11(20%) | Afcc kh(18%) |
| | | ip0.4 rri1+ | 00.4 mi | 00.4 m1 | 00.4 m1 | ip0.4 m1 |
| 2012 Jun. 4 | Day 20 | Afod raas 4(10%) | Afod raas 5(5%) | Afod raas | Afod kh(20%) | Ma; kh(18%) |
| | | ip0.4 m1 | 1130.4 rni | 6(25′300.4 rd | 1p0.4 rril | 00.4 m1 |
| 2012 Jun. 5 | Day 21 | Afod raas 4(10%) | Afod raas 5(5%) | Afod raas 6(25%) | Afod ith(20%) | Afcc kh(18°, 1) |
| | | ip0.4 m1 | ip0.4 rni | ip0.4 rni | 00.4 rni | 1p0.4 m1 |
| 2012 Jun. 6 | Day 22 | Afod raas 4(10%) | Afod raas 5(5%) | Afod raas 6(25%) | Afod ich(203f,) | Afcc kh(18%) |
| | | ip0.6 m1 | 00.6 rrii | 00.6 rr31 | ip0.6 rill | 00.6 m1 |
| 2012 Jun. 7 | Day 23 | Afod raas 4(10%) | Afod raas 5(5%) | Afod raas 6(25%) | Afod kh(20%) | Moe kh(18%) |
| | | ipB1D1 mi+ | ipB 1Di rni | ipB 1Di rni | ipB1D1 nil | ipEil D1 mi |
| 2012 Jun. 8 | Day 24 | Afod raas 4(10%) | Afod raas 5(5%) | Afod raas 6(25%) | Afod kh(20%) | Ma; kh(18%) |
| | | iplrni | ip 1 ml | ip 1 ml | ipl ml | p1 nil |
| 2012 Jun. 9 | Day 25 | | | | | |
| 2012 Jun. 10 | Day 26 | | | | | |
| 2012 Jun. 11 | Day 27 | | | | | |
| 2012 Jun. 12 | Day 28 | | | | | |
| 2012 Jun. 13 | Day 29 | + | Afod raas 5(5%) | | | |
| | | | 00.4 mi | | | |
| 2012 Jun. 14 | Day 30 | | Afod raas −− 5(5%) | | | |
| | | | 1130.4 rni | | | |
| 2012 Jun. 15 | Day 31 | | Afod raas 5(5%) | | Nod kh(20%) | Mee kh(18%) |
| | | | 00.8 m1 | | 00.81 [symbol] | 00.8 r [symbol] |
| 2012 Jun. 16 | Day 32 | | | | | |
| 2012 Jun. 17 | Day 33 | | Afod raas 5(5%) | | Afod kh(20%) | Afcc kh(18%) |
| | | | 00.8 rni | | 00.8 [symbol] | 00.8 rr [symbol] |
| 2012 Jun. 18 | Day 34 | | Afod raas 5(5′34) | | Afod kh(20%) | −− |
| | | | 00.4 mi | | ip0.4 | |
| 2012 Jun. 19 | Day 35 | | | | | |
| 2012 Jun. 20 | Day 36 | | | | | |

Note:
Day las the first dosing day. [symbol]
indicates data missing or illegible when filed

RAAS

Title: Anti-tumor efficacy of high concentrated fibrinogen enriched al at

thrombin and Afod (FS) in combination with Afod RAAS 2 or Afod RAAS 4 in patient-derived tumor xenograft (PDX) models in nude mice.

Description: Patient-derived liver tumor xenograft (PDX) partial removal model was used to evaluate the anti-cancer efficacy of high concentrated fibrinogen enriched al at thrombin and Afod (FS) in combination with Mod RAAS 2 at different 3 doses or with RAAS 4 at one dose. The results showed FS in combination with Afod RAAS 2 at all dosed or with RAAS 4 significantly inhibited the growth of remaining tumor at the beginning of treatment, but the duration was not long. On day 24 after dosing, the tumor sizes and tumor weights in FS in combination with Mod RAAS 2 groups or with RAAS 4 group were not significantly inhibited compared with sham-operated control group. In summary,

FS in combination with Afod RAAS 2 or RAAS 4 inhibited the liver PDX tumor growth temporarily.

Subjecthigh concentrated fibrinogen enriched al at thrombin and Afod (FS),

Afod RAAS, patient-derived tumor xenograft model, liver cancer

Summary

Patient-derived liver tumor xenograft (POX) partial removal model was used to evaluate the anti-tumor efficacy of high concentrated fibrinogen enriched al at thrombin (FS) in combination with RAAS 2 at 3 doses or with Mod RAAS 4 at one dose. The mice were

implanted subcutaneously with L1-03-0117 P6 tumors fragments of about 30 mm3. When xenograft tumors reached 200 mm3

a portion of tumor was removed by surgery, and a

portion of tumor of 20 mm3 in size was left, and FS or a control agent was applied to wound surfaces of both sides after tumor removal. Injection of Afod RAAS 2 or Mod RAAS 4 was conducted 2 days after the surgery, and lasted for 24 days. Tumor size and body weight were measured once per week. 24 days after injection of test agents, the mice were sacrificed and tumors were dissected and weighed. The tumor volumes and final tumor weights for all groups were statistically analyzed by one-way ANOVA with the significance level set at 0.05. The data showed that FS in combination with Mod RAAS 2 at all doses or with RAAS 4 significantly inhibited the growth of remaining tumor, but anti-tumor efficacy lasted less than 3 weeks. On day 24 after dosing, the tumor sizes and tumor weights in FS in cmnbination with Mod RAAS 2 at all dosed or with RAAS4 group were not significantly inhibited compared with sham-operated control group. In summary, FS in combination with Mod RAAS 2 or RAAS 4 inhibited the liver POX tumor growth temporarily.

TABLE OF CONTENTS
DETAILS OF FACILITY, PERSONNEL AND DATA

| | |
|---|---|
| LOCATION96,66Q9968Q996,86996,66Q1.9686996,66Q9968Q996,86996,66Q9968Q996,8699 | 157 |
| 6,66Q9968Q996,86996,66Q996 | |
| 2. INTRODUCTION6,P11,68Q99,5691.S66.P11,68Q99S66.P11,68Q99,5691.S6,P1.968.POOSSO.P11,68Q99,5691. | 157 |
| S6,/ | |
| 3, METHODS699968Q996,86996,66Q9968Q996,86996,66Q996,86996,66Q9968Q996,86 | 157 |
| 996,66Q9968Q996,66Q9968Q996,86996, | |
| 3.1.1. Animal preparation | 157 |
| 3.1.2. Tumor tissue preparation | 158 |
| 3.1.3. Formulation: | 158 |
| 3.2. Ex | |
| 3.2.1. Establishment of Xenograft Model and Treatment | 158 |
| 3.2.2. Evaluation of the Anti-Tumor Activity | 160 |
| 3.3. DRUGS, AND MATERTMs | 161 |
| 3.4. DATA ANALYSIS | 161 |
| 3.4.1. Relative Chage of Body Weight (RCBW) | 161 |
| 3.4.2. Tumor weight | 161 |
| 3.4.3. Statistical analysis | 161 |
| RESULTSevsaatesseatitsaatitsaatessaatitsaatesseatitsaatitsaatesseatitsaatesseatitsaatitsaatesseatit | 161 |
| saat | |
| 4′TUMOR GROWTH INHIBITION | 161 |
| ‘FELT ON BODY WEIGHT | 161 |
| 1 ISCUSSIONee4x.oe4*.ae44″ae44x.oe4*.ae44″ae44x.oe4*.ae44x.oe4*.ae44″ae44x.oe4*.ae44″a | 161 |
| e44x.e | |
| REFERENCESee4″aeo.x.oe.4″aeo.x.oe.*.aeo4″aeo.x.oe.*.aeo4″aeo.x.oe.*.aeo4″aeo.x.oe.*.aeo. | 163 |
| FIGURESaeo4″aeo.*.aeo4″aeo.x.oe.*.aeo4″aeo.x.oe.4″aeo.x.oe.*.aeo4″aeo.x.oe.*.aeo.x.oe.*.ae | 164 |
| o*aa | |
| FIG. L AiNT§.--TUMOR EFI-,1CACY OE FS+ AIoD EN′ PDX momi.11:1404117 | 164 |
| FIG. 2 ON HAY 24 V,TERTRIF-'s,TMENT | 164 |
| FIG. 3. PHOTOGRAPHS OF TUMORS EACH GPXX | 164 |
| FIG. 4. RELATIVE CHANG-17.. OF BOOY Vs/OF DIFFER | 164 |
| TABLESes..*.aeo4″aeo.x.oe.*.aeo4″aeo.x.oe.*.aeo4″aeo.x.oe.*.aeo4″aeo.x.oe.*.aeo4″aeo.x.oe.* | 165 |
| .aeo. | |

### 1. Details of Facility, Personnel and Data Location

The studies described in this report were carried out on behalf of RAAS at external laboratories:

### 2. Introduction

The aim of the study was to test anti-tumor efficacy of FS in combination with Afod RAAS 2 or Afod RAAS 4 in patient-derived liver tumor xenograft (PDX) partial removal model in nude mice.

The model used in the study was derived from surgically resected, fresh patient tumor tissues. The first generation of the xenograft tumors in mice was termed passage 0 (PO), and so on during continual implantation in mice. The passage of xenograft tumors at P7 (LI-03-0117) were used in this study.

All the experiments were conducted in the AAALAC-accrediated animal facility in compliance with the protocol approved by the Institutional Animal Care and Use Committee (IACUC).

### 3. Methods

3.1. Experimental Preparations

3.LL Animal preparation

Female Balb/c nude mice, with a body weight of approximately 20 grams, were obtained from an approved vendor (Sino-British SIPPR/BK Lab. Animal Co. Ltd., Shanghai, China). Acclimation/Quarantine: Upon arrival, animals were assessed as to their general health by a member of a veterinary staff or authorized personnel. Animals were acclimated for at least 3 days (upon arrival at the experiment room) before being used for the study.

Animal Husbandry: Animals \Nere housed in groups during acclimation and individually housed during in-life. The animal room environment was adjusted to the following target conditions: temperature 20 to 25° C., relative humidity 40 to 70%, 12 hours artificial light and 12 hours dark. Temperature and relative humidity was monitored daily.

All animals had access to Certified Rodent Diet (Sino-British SIPPR/BK Lab. Animal Co. Ltd., Shanghai, China) ad libitum. Animals were not fasted prior to the study. Water was autoclaved before provided to the animals ad libitum. Periodic analyses of the water were performed and the results were archived at WuXi AppTec. There were no known contaminants in the diet or water which, at the levels detected expected to interfere with the purpose, conduct or outcmne of the study.

3.L2. Tumor tissue preparation

The liver xenograft tumor models were established from surgically resected clinical tumor samples. The first generation of the xenograft tumors in mice is termed passage 0 (PO), and so on during continual implantation in mice. The tumor tissues at passage 7 (LI-03-0117) were used in this study.

3J 0.3. Formulation

High concentrated fibrinogen enriched alat thrombin and Mod were provide by RAAS and prepared by RAAS scientist during experiment before use. Matrigel (BD Biosciences; cat. #356234).

3.2. Experimental Protocol

3.2.1. Establishment of Xenograft Model and T:reatmenl

Grouping and treatment

Nude mice were assigned to 6 different groups with •15 or 25 mice/group and each group received different treatment as shown in Table i.

TABLE 1
Grouping and the treatment

| Group | Treatment | N | Surgery |
|---|---|---|---|
| 2 | Sham-operated | 15 | Remove 90% of tumor to keep 20 mm3, and close by |
| | control: | | suturing (no treatment). |
| | Positive control | 15 | Remove 90% of tumor to keep 20 mm3, treat the wound |
| | | | surfaces with Matrigel, and close by suturing. |
| 3 | AFOD RAAS2− | 25 | 6 Remove 90% of tumor to keep 20 mm3, treated the |
| | FFS --- high | | wound surfaces of both sides with Afod RAAS 2 × 10 |
| | | | (once every 1 minute for 10 times), and then with 3 times |
| | | | of FS (about 0.4m1), and close by suturing. |
| | | | 6 After 2 days, treat with Afod RAAS 2 (400 ul, QD |
| | | | x30, iv). |
| 4. | AFOD RAAS 2+ | 15 | Remove 90% of tumor to keep 20 mm3, treated the wound |
| | FS - moderate | | surfaces of both sides with Afod RAAS 2 × 8 (once every |
| | | | 1 minute for 8 times), and then with 2 times of FS (about |
| | | | 0.3 ml), and close by suturing. |
| | | | * After 2 days, treat with Afod RAAS 2 (300 ul, QD |
| | | | x30, iv). |
| 6 | AFOD RAAS 2+ | 15 | Remove 90% of tumor to keep 20 mm3, treated the |
| | FS - low | | wound surfaces of both sides with Afod RAAS 2 × 6 (once |
| | | | every 1 minute for 6 times), and then with 1 times of FS |
| | | | (about 0.2 ml), and close by suturing. |
| | | | 0 After 2 days, treat with Afod RAAS 2 (200 ul, QD |
| | | | x30, iv). |
| | AFOD RAAS2+ | 15 | Remove 90% of tumor to keep 20 mm3, treated the |
| | FS + RAAS 4 | | wound surfaces of both sides with Afod RAAS 2 × 10 |
| | | | (once every 1 minute for 10 times), and then with 3 times |
| | | | of FS (about 0.431), and close by suturing. |
| | | 100 | After 2 days, treat with Afod RAAS 4 (400 ul, QD x30, |
| | | | iv). |

Experiment procedures

A Xenograft tumors were collected and cut into pieces of 30 mm3 and implanted into 120 mice subcutaneously (with 30%) extra).

B. When xenograft tumors reach 200 mm3, the animal was anesthetized by i.p. injection of sodium pentobarbital at 60-70 mgikg. The animal skin was sterilized with ethanol solution. Skin was opened.

C. A portion of tumor was removed by surgery, and a portion of tumor of 20 mm3 in size was left for further growth.

D. Apply test agents or positive control agent locally following the study design.

OB gel shouldn't be used to avoid potential side effects. E. The skin was closed and sutured.

F. Pictures were taken in representative animals in each group, before and after surgical removal of tumor, and after completion of surgery.

G. Postoperative care was conducted by following SOP-BE0-0016-1.0.

H. Injection of AFOD RAAS 2 or AFOD RAAS 4 was conducted 2 days after the surgery, and lasted for 24 days.

I. During the period of the experiment, health conditions of mice were observed daily. Body weight of mice was monitored once per week.

J. Turnor sizes were measured once per week. Turnor volumes (mm3

were obtained

by using the following formula: volume=(W2×L)/2 (W, width; L, length in mm of the tumor).

K. Mice, which showed a significant loss of body weight (>20%), or which were unable to eat or drink, or exhibit ulceration on the skin/tumor, or the tumor size reached 2,000 mm3

were euthanized immediately to minimize the pain and distress. Such

actions need to notify the sponsor within 24 hrs (48 hrs during the weekends).

L. Mice were scarified at the end point (24 dafter injection of test agents).

a) Dissemination of cancer was identified macroscopically. The tissue surrounding tumor was also checked for the invasion of cancers.

b) Tumors were collected and their weights will be measured.

c) Pictures of collected tumors were taken.

3.2.2. Evaluation of the Anti-Tumor Activity

Health conditions of mice were observed daily. Body weights were measured once a week during the treatment. Tumor sizes were measured weekly. Tumor volumes (mm3 were obtained by using the following formula: volume:::: (W2×L)/2 (W, width; L, length in mm of the tumor). On day 14 after treatment, one mouse in Mod RAAS 2+FS--- high group was sacrificed due to tumor size reached more than 2,000 mm:3. On day 20 after dosing, one mouse in Afod RAAS 2+FS-moderate group died. On day 24 after treatment, all mice were sacrificed. Routine necropsy was performed to detect any abnormal signs of each internal organ with specific attention to metastases. Each tumor was removed and weighted.

3.3. Drugs and Materials

High concentrated fibrinogen enriched alat thrombin and Afod (FS), Afod RAAS2 and Mod RAAS 4 were provided by RAAS; Matrigel was from BD Biosciences (San Jose, Calif., cat. #356234).

Digital caliper was from Sylvac, Switzerland.

3.4. Data Analysis

3.4.1. Relative Chage of Body Weight (RCBW)

Relative change of body weight (RCBW) was calculated based on the following formula: RCBW (%)=(BWi−BWO)/BWO×100%; BWi was the body weight on the day of weighing and BWO was the body weight before surgery.

3.4.2. Tumor weight

Tumors weighed after sacrificing mice.

3.4.3. Statistkal analysis

Data were expressed as mean±SEM; the difference between the groups was analyzed for significance using one-way ANOVA and Dunnett's test

### 4. Results

4.1. Tumor growth inhibition

On 14 days after treatment, the tumor volume in vehicle group reached 1070 nHn3 on average, while tumor volume on average in Afod RAAS 2+FS-high, Afod RAAS 2+FS-moderate, Mod RAAS 2+FS-low and, Mod RAAS 4+FS groups was 663 mm3,596 mm3

640 mm3 and 531 mm3 respectively. On day 24 after dosing, the tumor size and tumor weight in FS combination with Afod RAAS 2 at all dosed or RAAS 4 groups was not significantly inhibited compared with sham-operated control group.

The inhibition on tumor growth were shown in FIG. 1-3.

4.2. Effect on Body weight

RAAS 2 groups or vvith RAAS 4 groupindicatinq tht:test a t: nt has no/Htt!e side eh\\: cts. The effect on body weight was shown in FIG. 4 and table 2.

### 5. Discussion

Patient-derived liver tumor xenograft (POX) partial removal model was used to evaluate the anti-cancer efficacy of FS in combination with Afod RAAS 2 at 3 doses or with Mod RAAS 4 at one dose. When xenograft tumors reached 200 mm:3, a portion of tumor was removed by surger and a pOliion of tumor of 20 mm3 in size was left for fU!iher growth, and FS or a control agent was applied to wound surfaces of both sides after tumor removaL The mice were treated 2 days after the surgery, and lasted for 24 days. On 14 days after treatment, the tumor volume in vehicle group reached 1070 mrn3 on average, while tumor volume on average in AFOD RAAS 2+FS-high, AFOD RAAS 2+FS-moderate, AFOD RAAS 2+FS-low and, AFOD RAAS 4+FS groups was 663 mm: \ 596 mm:\ 640 mm3 and 531 mm3 respectively, which demonstrated Afod RAAS 2+FS or Afod RAAS

4+FS significantly inhibited the tumor growth. But anti-tumor efficacy did not last long, after about a week (on day 24 after dosing) the tumor size and tumor weight in FS combination with Afod RAAS 2 at all dosed or RAAS 4 groups reached more than

2000 mm3 and exhibited no significant difference with sham-operated control group, indicating no significant inhibitory effects on tumor growth.

In summary, high concentrated fibrinogen enriched alat thrombin (FS) in combination with Afod RAAS 2 or RAAS 4 inhibited the liver POX tumor growth temporarily.

### 6. References

N/A

### 7. Figures

FIG. 136

Data are expressed as mean±SEM. *<0.05, **<O.o•1 vs sham group (one-way ANOVA and Dunnett's test).

FIG. 137

FIG. 138

Tumor was from each mouse of model L1-03-0117 and weighed. Scale bar, 1 em.

FIG. 139

Data are expressed as mean±SEM. Relative change of body weight (RCBW) was calculated based on the following formula: RCBW (%)=(BWi−BWO)iBWO×100%; BWi was the body weight on the day of weighing and BWO was the body weight before surgery.

### 8. Tables

TABLE 2
Relative change of body weight (‘.’41,

| | | | | −2 | −1 | 0 | 3 | 10 | 14 | 17 | 23 |
|---|---|---|---|---|---|---|---|---|---|---|---|
| | Days after | | RC | RC | RC | RC | RC | RC | RC | RC | RC |
| | treatment | | BW | BW | BW | BW | BW | BW | BW | BW | BW |
| Compounds | Group | | (%) | (%) | (%) | (%) | (%) | (c../c) | (%) | (%) | (9/0) |
| Sham− | 1 | Mean | 0.00 | −4.59 | 3.42 | −3.37 | 1.91 | 7.01 | 10.35 | 11.22 | 15, 66 |
| operated | | SD | 0.00 | 2.65 | 3.57 | 3.34 | 4, 13 | 5.79 | 5.24 | 6.25 | 7.94 |
| control | | SE | 0.00 | | | | | | 1.35 | | |
| Positive | | art | + | | | 0.86 | | −.7 | 99:′ | | |
| control | | h | | | | −) | 1037 | 1.49 | | 1.61 | 2.05 |
| | | | 0 | 0.68 | 0.92 | | 1.27 | , :i | | ri | 16.4 0 |
| | | | 0.0u | 6.07 | ,, I | i | 6 | 6 | | 8.84 | 9.1 |
| | | | 0.00 | 1, | I | | 1 | | | 9.28 | 2.35 |
| 2−FFS−−− high | 3 | Mean | AFOD | 1.52 | − | 0.37 | 0.7873, 27 | 5.66 | 9.65 | 11.0 | 18.29 |
| | | | RAAS | | | | | | | | |
| | | | 0.00 | | | | | | | | |
| | | SD | 0.00 | 2.64 | 2.52 | 2.77 | 2.99 | 2.85 | 3, 85 | 4.30 | 8.08 |
| | | SE | 0.00 | 0.51 | 0.48 | 0.53 | 0.58 | 0.55 | 0, 74 | 0.84 | 1.58 |
| AFOD RAAS | | | 0.00 | 2.71 | | | 1 35 | | h | 9.69 | 17.8 |
| 2 +FS− | | | 0.00 | 1.50 | .9− | . | 1 | 3.51 | 437 | | 6.14 |
| moderate | | | 0.00 | | 1 | 4 | 0.42 | 0.85 | 1.06 | 5 | 1.5 |
| AFOD RAAS | 5 | an | 0.00 | ls, le− | 0.90 | 0.53 | 3.43 | 5.18 | 8.30 | 11.07 | 15, 78 |
| 2 +FS−−−iow | | | | 0.50 | | | | | | | |
| | | SD | 0.00 | 4.30 | 3.63 | 4.22 | 4.38 | 4.94 | 5.48 | 6.95 | 10, 10 |
| | | SEM | 0.00 | 1.04 | 0.88 | 1.02 | 1.06 | 1.20 | 1.33 | 1.74 | 2.53 |
| AFC | 6 | an | , | 3.11 | −. | 4 | t | 3.23 | 6.036 | 8.50 | 8.98 |
| RAAS2 +FS+ | | | | | | | | | −03 | | 13.9 |
| RAAS 4 | | | | | | | | | 4 | | |
| | | | | | | | i − s, | | | ‘−! | |
| | | SD | (3.i−.) | 2 1 | ‘−’ | 1 | | | − | | |
| | | SE | −.) | 0.56 | ). | 4 | (3.84 | 0.82 | 1.17 | ..56 | 3.48 |

Relative change of body weight (RCBW) was calculated based on the following formula: RCBW (′Yo)=(BWi−BWO)/BWO×100%; BWi was the body weight on the day of weighing and BWO was the body weight before surgery.

FINAL REPORT

Characterization of lymphoid tissues and peripheral blood in nude mouse treated With and “\′vithout A FCC

TABLE OF CONTENTS

| | |
|---|---|
| 1. ABBREVIATIONS AND Dl!:FlNlTIONS | 97 |
| 2. INTRODlJCTION | 98 |
| 3. l)lJ ll.I)()Sl | 98 |
| 4. J\ili\TERii\LS | 98 |
| 5 I XPERl I\1EN′f l′\-tE′fl-1()1) | 99 |
| 6. DATi\ i\Ni\LYSIS | 103 |
| 7. I ESlJL′fS | 103 |
| 8. CONCLlTSION | 106 |
| OBJFCTJVE | ll 2 |
| 2.1′vfATEHlAI,S AND l′v1ETHOD | []2 |
| 2.1. Animals, reagents and instruments | 112 |
| 2.1.1 Animal Specifications | 112 |
| 2.1.2 Animal Husbandry | 112 |
| 2.1.3 Animal procedure | 113 |
| 2.1.4 Reagents and instruments | 113 |
| 2.2. Procedure and n1ethod | 113 |
| 2.2.1 4T1-1.UC cell culture | 113 |
| 2.2.2 Animal model establishment | 114 |
| 2.2.3 i′v1easurements | 115 |
| 2.2.4 Formulation preparation | 115 |
| 2.2.5 Animal experin1ent | 116 |
| 2.2.6 Experimental endpoint | 117 |
| 2.3 Statistical Analysis | 117 |
| 2.3.1 TGI (tumor growth inhibition, in percentage) | 117 |
| 2.3.2 T/C (?lo) calculation | 117 |
| 2.3.3 ANOVA analysis | 117 |
| 3. R.ESUJ.TS AND DlSCFSSlON | 11 X |
| 3.1 Turnor growth curve based on relative ROI | 118 |
| 3.2 Tumor growth curve based on tumor volume | 118 |
| 3.3 Toxicity evaluation by body weight change(%) monitoring and | 119 |
| daily observation of 4T1 • LUC-bearing Balb/c nude mice | |
| 3.4 TGT (′l) calculation | 120 |
| 3.5 T/C (%) calculation | 121 |
| 4. CONCLUSION | 121 |
| APPENDICES | 122 |
| EXHIBIT 1: FLUORESCENCE IMAGES OF THE WHOLE | 122 |
| BODY | |
| EXHIBIT 3: DAILY TESTING ARTICLES | 147 |
| RECORD | |

Executive Stnnmary

The purpose of this study was to investigate the effect of AFCC on curing tumor through characterizing distinct cell lineage in lymphoid tissues and peripheral blood in nude mouse treated with and without AFCC. Distinct cell lineage was differentiated by cell surface marker proteins. T cells, B cells, activated B cells, myeloid dendritic cell (mDC), plasmacytoid dendritic cell (pDC), granulocytes, and monocytes/macrophages were characterized.

In spleen and lymph nodes except in peripheral blood, AFCC treatment resulted in increased CD3+T cell population compared with that in nude mouse with tumor (FIG. 3, 9, 15). In spleen, lymph nodes. and peripheral blood, with AFCC treatment, B cell population together with activated B cells also increased compared with those in nude mouse with tumor (FIGS. 4, 10, 16, 5, 10, and 20). In spite of the increased cell number of B cells and T cells after AFCC treatment, granulocytes decreased (FIG. 7, 14, 18). Macrophages were found to decrease after AFCC treatment In peripheral blood and spleen but not in draining lymph nodes (FIG. 6, 13, 19). mDC and pDC percentages were not greatly affected in nude mouse in the presence of AFCC (FIG. 8, 11, 17).

### List of Abbreviations

| | | |
|---|---|---|
| FACTS | Flow Cytometry mDC | Myeloid dendritic cell pDC |

Plasmacytoid dendritic cell

Materials and Methods

Materials

Reagents

FITC, Rat Anti-Mouse CD4, BD, Cat: 557307

FITC, Rat Anti-MouseCD3 molecular complex, BD, Cat: 561798

PerCP-Cy5.5, Rat Anti--Mouse CD4, BD, Cat: 550954

PE, Rat Anti-MouseB220/CD45R, BD, Cat: 553089

APC, Rat Anti-MouseCD: Ub, BD, Cat: 553312

APC, Ar Ham Anti-MouseCD11c, BD, Cat: 550261

PE, Rat Anti-MouseGR-1(Ly-6G and Ly-6C), BD, Cat: 553128

Purified, Rat Anti-MouseFc blocker CD16/32, BD, Cat: 553141

APC, Ar Ham Rat Anti--MouseCD69, BD, Cat: 560689

7-AAD, BD. Cat: 559925

ACK Lysing buffer, Invitrogen, Cat: A10492-01

PBS, Dycent Biotech (Shanghai) CO., Ltd. Cat: BJ141. FBS, Invitrogen Gibco, Cat: 10099141 l'Vlaterials

Cell strainer (70flm), BD, Cat: 352350

BD Falcon tubes (12×75 mm, 5 ml), BD, Cat: 352054

Equipments

Vi-CELL Cell Viability Analyzer, Beckman Coulter, Cat: 731050

FACSCalibur flow cytometer, BD, Cat: TY1218

Methods

Cell isolation and staining

Peripheral blood was collected through cardiac puncture. After removing red blood cells with lysis buffer followed by two rounds of washing using 1×PBS, mononuclear cells (monocytes, macrophages, dendritic cells, and lymphocytes) and granulocytes were obtained. Spleen and lymph nodes cell suspension were also obtained after filtering through 70flrn cell strainer. Cell viability and number were analyzed by Vi-CELL Cell Viability Analyzer. Cell surface labeling was performed after that. Blocked with Fe blocker CD16/CD32 at 49 C for 15 min, cells were centrifuged and resuspended in staining buffer (0.08% NaN3/PBS+1% FBS). Fluorescent-conjugated antibodies were then added into

the suspension at the indicated dilution according to the antibody usage protocol from the company. After 30 min incubation at 4 Q (for 30 min in the dark, cells were washed twice with 0.08% NaN3/PBS (200 fll per sample}, and resuspended with 400 fll 0.08% NaNjPBS in BD Falcon tubes (12×75 mm, 5 rnl) followed by FACS analysis.

Data analysis

FACS data were analyzed by flowjo softvvare.

Study Summary

Study initiation date and completion date

The study was initiated and finished on Apr. 13, 2012.

Study purpose

The purpose of this study was to investigate the effect of AFCC on curing tumor through characterizing distinct cell lineage in lymphoid tissues and peripheral blood in nude mouse tTeated vvitb and \vithout AFCC.

Study results

l′Vlice information

All the mice were transferred from oncology team from vVuxi Apptec. FIG. 1 and FIG. 2 contained the treatment and age information of the mice.

1: Nude m.ice with tumor: nude mice grafted vvith MDA-MB-231-Luc tmnor cells as vehicle for the study.

FIG. 140

10 nude mice from group 2-5 which have been implanted with tumor cells from the 2-5 mice positive control group using Docetaxel in another study done at another CRO lab.

FIG. 141

3: One of the 10 nude mice with MDA-MB-231-Luc tumor cells transferred from 2-5 positive control group using Docetaxel and it is used as positive control for the re-implantation study,

FIG. 142

Graph showing the tumor volume of Mice #6-10 from the study done from Jul. until Nov. 11, 2011 when the dead body of mouse #6-10 was removed from one CRO lab to another one for further study.

FIG. 143

Mouse #6-10 taken from Aug. 23, 2011 to November 3n1 2011 showing the growth of the tumor which had been detached from the body was under recovery from breast cancer using AFCC proteins for treatment.

FIG. 144

The tissue from the area of mouse #6-1 0 vvhere the tumor had been detached \vas used to implant in the 10 nude mice 66 days after re-implantations show no tumor growth.

FIG. 145

After 66 days lvith no growth, then we implanted the cancer tumor for a second time. The growth of the tumor in mice 6-10 which had been treated prior with AFCC at another CRO lab after re-implantation on Nov. 11, 2011.

FIG. 146

Graph showing 5 groups of nude mice after tumor volume change atler the second re-implantation with the breast tumor cancer, including mice #6-10 and mice #2-10 treated with Docetaxel.

FIG. 147

The picture of the 1 0 mice in group #6-10 showing mice #5-1 and mice #5-3 growing the tumor after second re-implantation both had been treated with AFCC on Feb. 29, 2012.

FIG. 148

2: Nude mice with AFCC treatm.ent:

Grafted with tumor cells numbered #6-10 starting at Nov. 11, 2011; received \vith AFCC provided by RAAS though I.V. or J.P. injection from Feb. 29, 2012. In April mice #6-10 with the second re-implantation has been completely recovered due to the AFCC proteins 'lvhich contain good healthy cells which sent signal to the DNA of the infected mice with breast cancer tumor, to transform the RNA to synthesize good proteins against the breast cancer eel L

FIG. 149.

Among the groups in the study for breast cancer from mid-Jul. to Nov. 11, 2011 nude mouse #4-6 has shown the quickest recovery period within 24 days. From day 15 when the tumor started to grow to day 39 when the tumor detached from the body.

FIG. 150

Mouse #4-6 grew the tumor on August 23rd and self-detached from the body September 18\2011.

FIG. 151

Mouse #4-6 on October 18th completely recovered from breast cancer due to the i\FCC KH protein which contains good healthy cells which sent signal to the DNA of the infected mice with breast cancer tumor, to transfonn the RNA to synthesize good proteins against the breast cancer celL

FIG. 152

The 9 mice from the #4-6 group first re-implantation of the tumor which had never grown and one of these mice #4 was used in this study for analysis of the cells.

FIG. 153

4: Nude mouse with no tumor: grafted with tumor cells numbered #4-6 starting at Nov. 18, 2011, no further treatment needed due to failure of the tumor grmvth because good healthy cells fi•orn the AFCC treated, which contains good healthy cells which sent signal to the DNA of the infected mice with breast cancer tumor, to transform the RNA to synthesize good proteins against the breast cancer cell.

FIG. 154

5: Nude na″ive mouse at 8 weeks old was used as a negative normal control to determine the normal nude mice cells.

FIG. 155

6: C57BL/6 mouse at 8 weeks old was used as a negative normal control to determine the normal nude mice cells.

FIG. 156

Cell population in peripheral blood

After whole blood withdrawal, distinct cell lineage was differentiated by cell surface marker proteins. T cells, B cells, activated B cells, mDC, pDC, granulocytes, and monocytes/macrophages were characterized {FIG. 3 to FIG. 8).

As shown by FIG. 3, AFCC treatment didn't affect CD3+T cell population compared with that In nude mouse with tumor and without tumor. After AFCC treatment, B cell population, on the other hand, increased to the similar percentage as seen in nude

mouse no tumor and nude na′ive mouse, suggesting the potential effect of AFCC on B cell lineage (FIG. 4). Activated B cells also increased with AFCC treatment, which was illustrated in FIG. 5. Macrophages and granulocytes decreased after AFCC treatment compared with those in nude mouse with tumor (FIG. 6 and FIG. 7). Nude mouse no tumor and nude mouse with AFCC treatment had similar mDC and pDC percentage shown in FIG. 8.

FIG. 157

FIG. 158

FIG. 159

FIG. 160

Cell population in spleen

Distinct cell lineage in spleen cell suspension was further characterized by cell surface marker proteins. T cells, B cells, activated B cells, mDC, pDC, granulocytes, and monocytes/macrophages were included (FIG. 9 to FIG. 14).

As shown by FIG. 9, AFCC treatment slightly increased CDJ′T cell population compared with that in nude mouse with tumor and nude mouse without tumor. After AFCC treatment, B cell population, on the other hand, increased to the similar percentage as seen in nude mouse no tumor, suggesting the potential effect of AFCC on B cell lineage (FIG. 10). Activated B cells also increased with AFCC treatment, which was illustrated

in FIG. 12. Macrophages and granulocytes dramatically decreased after AFCC treatment compared with those in nude mouse with tumor (FIG. 13 and FIG. 14}. Nude mouse no tumor and nude mouse with AFCC treatment had similar mDC and pDC percentage shown in

FIG. 11.

FIG. 161

FIG. 162. FIG. 163

FIG. 164

FIG. 165

FIG. 166

Cell population in draining lymJlh nodes

Distinct cell lineage in draining lymph nodes suspension was further characterized by cell surface marker proteins. T cells, B cells, activated B cells, mDC, pDC, granulocytes, and monocytes/macrophages were included (FIG. 15 to FIG. 20).

As shown by FIG. 15, AFCC treatment dramatically increased CD3_,_T cell population compared with that in nude mouse with tumor. T cells in nude mouse with AFCC treatment and mouse no tumor had the similar percentage (FIG. 15). After AFCC treatment, B cell population, on the other hand, increased to the similar percentage as seen in nude mouse no tumor, suggesting the potential effect of AFCC on B cell lineage (FIG. 16). Activated B cells also increased with AFCC treatment, which was illustrated in FIG. 20. Granulocytes dramatically decreased after AFCC treatment compared with those in nude mouse with tumor and na'ive nude mouse (FIG. 18). mDC and pDC also decreased in the presence of AFCC compared to those in nude mouse with or without tumor (FIG. 17). Macrophages still maintained the similar percentage with and without AFCC treatment (FIG. 19}.

FIG. 167

FIG. 168

FIG. 169

FIG. 170

FIG. 171

FIG. 172

### 7 Conclusions

The effect of AFCC on curing tumor through characterizing different cell lineage in lymphoid tissues and peripheral blood in nude mouse was investigated using staining with different marker proteins for distinct cell lineages followed by FACS. T cells, B cells, activated B cells, mDC, pDC, granulocytes, and monocytes/macrophages were characterized in 6 mice illustrated in FIG. 1 and FIG. 2.

FACS analysis showed that AFCC treatment had the effect on the population of major cell lineages in immune system. Increased CDJ′T cell population was found in nude mouse treated with AFCC compared with that in nude mouse with tumor in spleen and lymph nodes (FIG. 9, 15). B cells including activated B cells also increased compared with that in nude mice with tumor in spleen, lymph nodes, and peripheral blood (FIG. 4, 10, 1.6.

5, 10, 20}. Granulocytes and macrophages, however, were found to decrease after AFCC treatment in peripheral blood and spleen (FIGS. 7, 14, 18, 6, 1.3, and 19). The decrease as one of the lymphocytes, white blood cells. which are present in the peripheral blood of the nude mice with the breast cancer cell proves that the vehicle and positive control mice when the breast tumor grew the cancer cell have affected the peripheral blood.

Even though the mice has not been metastasized. This make the inventor to believe that any cancer tumor grow the cancer cells are already in the peripheral blood.

KH good healthy cells 1—Send signals to the DAMAGED, SICK, AND BAD CELLS that triggers

that synthesis of good proteins that transform these cells to become GOOD healthy cells; 2-Send signals to the other currently undamaged cells to synthesis of good proteins to protect them from being DAMAGED, INFECTED and PRONE to DNA and other cellular alterations; 3 Send signals to the body to produce new cells that are healthy and forbid them from being affected by intra- and extracellular damaging signals in order to cure diseases, viruses infections, bacteria infections, auto immune disease. neurological disorder, all type of solid and blood cancer, coagulation, diabetic, inhibitor, immune deficiency, muscle and nerve repair and restoration.

Macrophage have been found to decrease after AFCC treatment in peripheral blood and spleen. But it has not decreased in the vehicle and positive control mice. According to the text books Macrophage is the big eater which consumes all bad and damaged cells and because of this they become sick or damaged. The level of Macrophage In the vehicle or positive control increase as they RNA of the bad damaged cells are synthesizing a bad protein that causes cancer. While KH good healthy cells synthesize good proteins against the breast cancer.

Taken together, this study suggests the effect of AFCC on curing tumor through changing the population of major cell lineages in immune system, including spleen, lymph nodes and peripheral blood.

Report: Antiviral efficacy of AFOD RAAS!R2 in an influenza H1N1 . . . infected mouse model

Report No: WX IFV05222012

Issue Date: Jun. 13, 2012

Study No: RAAS 05222012

Study Period: May″ 221 2012 to Jun. 8, 2012

Content

Summary of the report

Objective

Infection with human influenza virus (IFV) causes respiratory tract illness in human and animals including mice. Mouse model intranasally infected with IFV H1N1 is well recognized for antiviral compound screening against IFV infection. This study is designed to evaluate the compound AFOD RAAS2 from RAAS for its in vivo anti-IFV efficacy.

Study Method

This study was peliormed in the following steps:

1) Infect mice with IFV by intranasal inoculation.

2) Treat the mice pre or post INF infection by iv/ip dosing of the AFOD RAAS2. 3) Daily record body weight of the mice.

4) Sacrifice survived mice and inspect their major organs in the end of the study. Result

Summary

One-week preventive treatment with RAAS-2 fully protected H1N1-challenged mice from death and body weight loss although one-week therapeutic treatment with RAAS-2 led to one mouse, out of 5 mice survived in this group to the end of the experiment. In the H1N1-challenged vehicle control group all mice died and their body weights dramatically dropped by 20% to 30% within 4-7 days post-IFV H1N1 challenge. In contrast with the vehicle group, all mice treated therapeutically with oseltamivir survived although their body weights dropped and recovered to some extent. This indicated that the mouse model worked successfully in current study.

For Study Protocol: RAAS 20120428.v.2

I. Method

Animals:

Female BALB/c mice (6-8 weeks, 17-22 g) were divided into defined study groups after a visual examination and a 3 to 5-day acclimation upon arrival.

Solution preparation:

1. Sodium Pentobarbital: Freshly dissolved in saline for injection at 7.5 mg/ml prior to using.

2. Test article: human plasma derived protein 29% AFOD RAAS2 in sterile solutions for vein injection provided by the client.

3. Vehicle: PBS

4. Oseltamivir phosphate (prodrug): aqueous solution in PBS, 0.1 mg/ml

Experimental Procedure:

IFV infection and test article administration:

1, From day −7 through day −1, 5 mice from group 4 are intravenously or intraperitoneally (iv/ip) administrated daily for 7 days.

2. On the day of Influenza administration, mice are anesthetized by intraperitoneal injection of sodium pentobarbital (80 mg/kg).

3. Anesthetized mice are inoculated with 5×10″3 pfu/mouse of Influenza H1N1 A/WSN/33 via the intranasal route in SFM medium.

4. Test article or vehicle is intravenously or intraperitoneally (iv/ip) administrated daily for 7 days. Oseltarnivir (1 mg/kg) is orally given twice daily for 8 days. First dosing for oseltarnivir or test article is executed 4 h pre H1N1 inoculation.

5. From day 1 through day 14 the infected mice are observed two times a day. Mortality and body weight are recorded daily.

6. On day 14, all living mice are sacrificed and dissected for the inspection of organ appearances.

II. Groups and schedules:

Table 1 Action summary of the Study

TABLE 1
Action summary of the Study

| | | | 1FV | AFOD, | | | |
|---|---|---|---|---|---|---|---|
| | | | challenge, | iylip, | Oseltamivir, | po | |
| Study | | | 14:00- | 10:00- | 10:00- | 19:50- | mouse |
| Day | Date | Weighing | 16:00 | 12:00 | 10:20 | 20:10 | sacrifice |
| Day-7 | May 22, 2012 | −\/ | | N1+ | | | I |
| Day-6 | May 23, 2012 | | , j | | | | |
| Day-5 | May 24, 2012 | −\, / | | .., 1 | | | |
| Day-4 | May 25, 2012 | −\/ | | Ni+ | | | I |
| Day-3 | May 26, 2012 | , j | | ′ | | | |
| Day-2 | May 27, 2012 | | −\/ | | | | |
| Day-1 | May 28, 2012 | −\/ | | Ni | | | |
| Day 0 | May 29, 2012 | , j | Ni | ‘+ | Ni | | I |
| Day 1 | May 30, 2012 | −\/ | | | −, / | | |
| Day 2 | May 31, 2012 | −\/ | | v′ | | NI | |
| Day 3 | Jun. 1, 2012 | , j | | ‘+ | Ni | | I |
| Day 4 | Jun. 2, 2012 | −\/ | | .., 1 | −, / | ,, j | |
| Day 5 | Jun. 3, 2012 | −\/ | | v′ | | NI | |
| Day 6 | Jun. 4, 2012 | , j | | +I | Ni | | |
| Day 7 | Jun. 5, 2012 | −\/ | | | | | |
| Day 8 | Jun. 6, 2012 | −\/ | | | | | |
| Day 9 | Jun. 7, 2012 | ,, i | | + | | | I |
| Day 10 | Jun. 8, 2012 | −\/ | | | | | |
| Day 11 | Jun. 9, 2012 | −\/ | | | | | |
| Day 12 | Jun. 10, 2012 | | | d+ | | | I |
| Day 12 | Jun. 11, 2012 | −\/ | | | | | |
| Day 13 | Jun. 12, 2012 | −\/ | | + | | | I |
| Day 14 | Jun. 13, 2012 | d | | | | | −v, |

indicates ti−)at the action was taken.

TABLE 2
ExperimentalDesign for the efficacy study

| | | | | | | 1st | |
|---|---|---|---|---|---|---|---|
| | | | | Vol | Treatment | treatment | H1N1 |
| Group | Mice | Compound | Dose | (ml/kg) | Schedule | time | (PFU/mouse) |
| | 5 | Vehicle | 0.2/0.4 | −− | Iv/ip, QD* | 4 hrs pre- | 5x1_0A3 |
| | | | mi/mouse | | | infection | |
| 2 | 5 | AFOD RAAS 2 | nil/mouse | −− | 0.2/0044 | hrs | 5x10A3 |
| | | | | | lviip, QUA | pre- | |
| | | | | | | infection | |
| 3 | 5 | Oseltamivir | 1 nig/kg | phosphatepre- | p0, BID″ | 4 | 5x 1 0A3 |
| 4 | 5+ | AFOD RAAS | 0.2/0.4 | infection | Iv/ip, QD* | 7 days | |
| | | | milmouse | 10 | 5x10″′32 | pre- | |
| | | | hrs | −− | | infection | |

lv/ip, OD*: Iv/ip means that iv injection is carried out with the volume indicated in “dose” column on day 0, 1, 2, 4 and ip injection is carried out on day 3; QD: daily (QD) for 4 days after H1N1 inoculation;
**BID, twice daily.
Vehicle: PBS

BI Adverse Events and Tolerability of Compounds:

1. On day 5 post H1N1 infection, hematuria occurred in group 2 of AFOD RAAS2 treatment.

We stopped AFOD RAAS2 medication on the sixth day post H1N1 infection.

2. One mouse in the oseltamivir group died day 3 post H1N1 challenge. Its body dissection indicated that its esophagus was damaged probably due to harsh oral gavage.

Therefore this mouse was ruled out from the experiment

Result and discussion

In the H1N1-challenged vehicle control group all 5 mice died and their body weights dramatically dropped by 20% to 30% within 4-8 days post-IFV H1N1 challenge (FIG. 1, FIG. 2, and Table 3). In contrast with the vehicle group, 4 out of 5 mice in the oseltamivir group survived to the end of experiment (FIG. 1, FIG. 2, and Table 3) although one mouse died accidentally of harsh oral gavage, which should be ruled out from the experiment as suggested early (see Part Ill, 2

in this report). The body weights in this group dropped by <15% days 5 to 8 post HI N1 challenge and recovered thereafter to some extent (FIG. 2). This indicated that the mouse model worked successfully in current study.

Impressively one-week preventive treatment with 0.2 ml/0.4 ml/mouse iv/ip QD of RAAS-2 totally protected HI N1-challenged mice from death and body weight loss till the end of this study (Fig I, FIG. 2 and Table 3). The protection of body weight loss by the preventive treatment of RAAS-2 is even better than that by oseltamivir treatment (FIG. 2). However the therapeutic treatment with 0.2 ml/0.4 ml iv/ip QD of RAAS-2 only protected one mouse out of 5 mice in the group from death and partial body weight loss of all 5 mice days 2 to 5 post H1N1 infection. Other 4 mice in this group died days 4 to 6 post H1N•1 infection. In addition, some of the mice in

status.

We don't understand why the RAAS-2 displayed such significant preventive efficacy on mouse death and body weight loss caused by H1N•1 challenge. We have a number of suggestions to fully establish and understand this efficacy. First, we need to expand the efficacy experiment using a few more mice each group to confirm the data due to the small experiment scale (5 mice each group only) in the current study. In addition, a longer term study should be designed to fully know how long the preventive efficacy of the blood-derived product RAAS-2 could last For example the mice should be challenged with H1N1 two weeks, three weeks, four weeks and even longer, respectively, post one week of preventive treatment of the RAAS-2. Some well designed mechanism studies should be carried out, such as in vivo H1N1 replication in infected mouse lungs in the preventive treatment and control groups, detection of immunological markers to reflect immune system activation and other biomarker assays post preventive treatment and H1N1 challenge. Finally a dose-dependent observation should be carried out for the RAAS-2 preventive treatment.

FIG. 173. Effect of AFOD RAAS2 on H1N1M caused mouse mortality

TABLE 3
Effect of AFOD RAAS2 or Oseltamivir on mean day to death (MOD) of
mice infected with H1N1 A/WSN/33

| | | Survivor/ | Mean day to |
|---|---|---|---|
| Treatment | Dose | total | death ± S.D. |
| H1N1 + Vehicle | 0.2/0.4 nil/mouse | 0/5 | 4.8 ± 1.3 |
| H1N1 + AFOD | 1 mg/kg | 1/5 | 6.2 ± 4.4 |
| RAAS2 | | | |
| H1N1 + Oseltarnivir | 0.2/0.4 ml/mouse | 4/4 | >14 ± 0.0*** |
| AFOD RAAS2-4- | 0.2/0.4 ramouse | 5/5 | >14 ± 0.0* |
| H1N1 | | | |

***P <:0.001 compared to the H1N1 + vehicle control

FIG. 174. The average body weight change in mice infected with H1N1 influenza

### APPENDIXES

The scanned primary in vivo experiment records of study RAAS 04242012 are attached. File name: Primary in vivo Experiment Record of Study RAAS 04242012

Effects of AFOD on 6-OHDA rat model of Parkinson's disease

I. General Information
Experiment requested by: Mr. Kieu Hoang from Shanghai RAAS Project ID I code: RAAS/PD2k′11-01

Experimental objective: To study the effects of AFOD on 6-OHDA lesioned rat model of Parkinson's disease

Target start date: Jul. 18, 2011

II. Sample Information

Sample description: AFOD: Liquid, the concentration is 5%, store at 4° C.

Ill. Introduction

The objective of this study was to determine if there were any neuroprotective or regeneration effects of AFOD on 6-OHDA lesioned rat model of Parkinson's disease. Behavioral tests (cylinder test, adjusting step test and rotation test) and tyrosine hydroxylase (TH) staining were used for evaluating the locomotive performance of the animals and survival of dopaminergic neurons.

IV. Experimental Design

| | | | | | | | | Behavioral | |
|---|---|---|---|---|---|---|---|---|---|
| | | | | Frequency | Drug tax in | | | tests | |
| | No. | | | (every 3 | relation | 6-OH DA | Dose | &Video | IHO |
| GrouP | Animal | Sample | Route | days) | with lesion | lesion | (glkg) | Recordings | (TH) |
| A | 10 rats | diluents | IV | Day 1, | Pre | Day 15 | 09/kg | 2 weeks | After |
| | | | | 4, 7, 10, 13 | | | | after 6- | riehavioral |
| | | | | | | | | OHDA | tests |
| B | 10 rats | AFOD | IV | Day 1, | Pre | Day 15 | 0.5 g/kg | 2 weeks | After |
| | | | | 4, 7, 10, 13 | | | | after 6- | behavioral |
| | | | | | | | | OHDA | tests |
| C | 10 rats | AFOD | IV | Day 1, | Pre | Day 15 | 0.259/kg | 2 weeks | After |
| | | | | 4.7, 10, 13 | | | | after 6- | behavioral |
| | | | | | | | | OHDA | tests |
| D | 10 rats | AFOD | IV | Day 1, | Pre | Day 15 | 0.125 g/kg | 2 weeks | After |
| | | | | 4, 7, 10, 13 | | | | after 6- | behavioral |
| | | | | | | | | OHDA | tests |
| E | 10 rats | diluents | IV | Day 1, | post | Day 1 | O g/kg | 2 weeks | After |
| | | | | 4, 7, 10, 13 | | | | after 6- | behavioral |
| | | | | | | | | OHDA | tests |
| F | 10 rats | AFOD | IV | Day 1, | post | Day 1 | 0.5 g/kg | 2 weeks | After |
| | | | | 4, 7, 10, 13 | | | | after 6- | behavioral |
| | | | | | | | | OHDA | tests |
| | 10 rats | AFOD | IV | Day 1, | post | Day 1 | 0.25 g/kg | 2 weeks | After |
| | | | | 4, 7, 10, 13 | | | | after 6- | behavioral |
| | | | | | | | | OHDA | tests |
| H | 10 rats | AFOD | IV | Day 1, | post | Day 1 | 0.1259/kg | 2 weeks | After |
| | | | | 4, 7, 10.13 | | | | after 6- | behavioral |
| | | | | | | | | OHDA | tests |
| | 10 rats | diluents | IV | Day 1, | Pre + post | Day 15 | 09/kg | 2 weeks | After |
| | | | | 4, 7.10, 13.16, | | | | after 6- | behavioral |
| | | | | 19, 22, 25, 28 | | | | OHDA | tests |
| J | 10 rats | AFOD | IV | Day 1, | Pre + post | Day 15 | 0.59/kg | 2 weeks | After |
| | | | | 4, 7, 10, 13, 16, | | | | after 6- | behavioral |
| | | | | 19, 22, 25, 28 | | | | OHDA | tests |
| K | 10 rats | AFOD | IV | Day 1, | Pre + post | Day 15 | 0.25 g/kg | 2 weeks | After |
| | | | | 4, 7, 10, 13, 16, | | | | after 6- | behavioral |
| | | | | 19, 22, 25, 28 | | | | OHDA | tests |
| L | 10 rats | AFOD | IV | Day 1, | Pre + post | Day 15 | 0.125 g/kg | 2 weeks | After |
| | | | | 4, 7, 10, 13, 16, | | | | after 6- | behavioral |
| | | | | 19.22, 25.28 | | | | OHDA | tests |

V. Methods

1. Animals: male SO rats were purchased from Shanghai Laboratory Animal Center (SLAC). They were housed under 21-23 OC, with 12 h light-dark life cycle. Food and water were given ad libitum.

2. 6uOHDA lesion: Rats were anesthetized with 60 mg/kg sodium pentobarbital. They were stereotaxic injected with total dose of 20 pg of fresh prepared 6-OHDA (dissolved in saline containing 0.05% ascorbic acid, calculated as free base) into tvvo sites of the left striatum, using the following coordinates (in mm relative to Bregma): AP+i 0.0, L −2.5, DV −5.0; AP −0.4, L −4.0, DV −5.5. The injection rate was i pi/min and a total of 2 iJI was injected at each site. The needle was left in place for 3 min before retracting.

3. Cylinder test: Rats were placed in a transparent cylinder (22 cm in diameter and 30 cm height). Animal would rear and support its body with one or both of its forelimbs. Numbers of left, right or both forelimb(s) wall contacts were countered until total number of wall contact reached 20. Each behavioral was expressed as percent use of left, right or both limb(s) relative to the total number.

4. Adjusting step test The rats were held by the experimenter fixing the hindlimbs and slightly raising the hind pal oi f the body. The forelimb not to be tested was also fixed, with only the other forepaw touching the table. The rat was moved slowly sideways (90 cm in 5 s), first in the forehand (defined as right paw to the left and left paw to the right) then in the backhand (defined as right paw to the right and left paw to left) direction. The number of adjusting steps of each left and right forelimbs on both directions was recorded individually.

5. Apomorphine induced rotation test After completing the above two tests, rats were placed in a round container of 40-cm diameter. After 10-min acclimation, they were injected s.c. with 0.25 mg/kg apomorphine which induced spontaneous contralateral rotations. The number of contralateral rotation was countered for 5 min.

6. TH staining: After the completion of behavioral tests, animals were sacrificed with an over dose of pentobarbital and transcardiac perfusion fixed with 4% paraformaldehyde in 0.1M phosphate buffer (pH?0.4). Brains were removed and further fixed in the same fixative overnight at 4° C., they were transferred to 30% sucrose solution till sunk and then cut into 301Jm coronal sections on a cryostat microtome. Three sections of caudal, center and rostral part of the SN (bregma −5.5, −5.25 and -5.0 mm) were used for staining. The sections were incubated with primary antibody (TH, 1:1000, from Millipore) overnight at 4° C. followed by HRP-conjugated secondary antibody (Jackson lmmnoresearch). The sections were developed using diaminobenzidine as the chromogen. Sections were digitally captured through an Olympus DP72 camera connected to the microscope. Number of positively stained cells in the left and right sides of SN in each section was counted to make the summation. The ratio of left/right was calculated.

7. Statistic analysis: Data were expressed as mean±SEI\tl and analyzed with ANOVA followed by Tukey test. Significance level was set at p<0.05.

VI. Results

The study of post groups was stopped after three injections following the sponsor's request. There were one rat in pre control group, one in pre low dose group and two in pre-post control group died during lesion surgery. Other animals recovered well after lesion and continuous injection did not cause any obviously abnormal activities by normal clinical observation.

1. Effects of pretreatment of AFOD on the behavioral performance

Rats were treated with vehicle or AFOD of three different doses for 2 weeks before the 6-0HDA lesion. Behavioral tests were performed 2 weeks after lesion. All the four groups showed significant decline of right forepaw step in forehand direction (FIG. 1A). In cylinder test, they also showed significant declined right forepaw use (FIG. 1C). Injection of apomorphine induced obvious rotation in control, moderate and high dose groups, however the rotation of low dose group \Nas slightly less (FIG. 1D).

Data of the three tests were analyzed by ANOVA, there was no significant difference among groups.

FIGS. 175A-D. Effects of pretreatment of AFOD on the behavioral performance. Rats were treated with vehicle or AFOD of three different doses for 2 weeks before the 6-OHDA lesion. Behavioral tests were performed 2 weeks after lesion. A. Adjusting step test forehand direction. B. Adjusting step test backhand direction. Number of steps was counted when the rats were moved sideways. C. Cylinder test. Rats were placed in a cylinder and number of left, right or both forelimb wall contacts was countered. The behavioral results were expressed as percent use relative to the total number. D. Apomorphine induced rotation. Rats were injected s.c. with 0.25 mg/kg apomorphine and rotation was counted for 5 min. Data were expressed as mean±SEM. *p<0.05.

2. Effects of pretreatment+posHreatment of AFOD on the behavioral performance

Rats were treated with vehicle or AFOD of three different doses for 2 weeks before the 6-OHDA lesion. They were further treated for 2 weeks after lesion, and then behavioral tests were performed. All the four groups showed significant decline of right forepmN step in forehand direction (FIG. 2A). In cylinder test, they also showed significant declined right forepaw use (FIG. 2C). Injection of apomorphine induced obvious rotation in all the four groups (FIG. 2D). Data of the three tests were analyzed by ANOVA, there was no significant difference among groups.

FIGS. 176A-D. Effects of pretreatment+post-treatment of AFOD on the behavioral performance.

Rats were treated with vehicle or AFOD of three different doses for 2 weeks before the 6-OHDA lesion. They were further treated for 2 weeks after lesion, and then behavioral tests were performed. A Adjusting step test forehand direction. B. Adjusting step test backhand direction. Rats were held and let one forelimb touch the table. Number of steps was counted when the rats were moved sideways. C. Cylinder test. Rats were placed in a cylinder and number of left, right or both forelimb wall contacts was countered. The behavioral results were expressed as percent use relative to the total number. D. Apomorphine induced rotation. Rats were injected s.c. with 025 mg/kg apomorphine and rotation was counted for 5 min. Data \Nere expressed as mean±SEM. *p<0.05.

3. TH staining

To verify the neuron survival in the SN, five rats from each group (except pre low dose group that all the nine rats were sacrificed) were perfused for fixation after the behavioral tests and IHC staining of TH was performed. In control group, there was 30%-40% neurons survival in the lesion side (left side). Pre low dose group had less neurons remained in the lesion side, however there was no significant difference by ANOVA analysis.

FIGS. 177A-B. TH staining of the SN. Rats were perfused and the brains \Nere fixed for IHC study.

Three sections from caudal, center and rostral part of the SN (bregma −5.5, −5.25 and -5.0 mm) of each brain were used for staining. Cell number of each side was counted and the ratio of left/right was calculated. Data were expressed as mean±SEM.

4. Results from daily injected rats

The rest of the rats of pre and pre/post groups were selected for further treatment of AFOD. The treatment protocol was shown in table •1:

TABLE 1
Protocol for daily injection

Cage I Rat No. I Dose
-----------------------------------------------------------------------------------------
---------

| | |
|---|---|
| 4̅′̅ | AFOD: 1O ml/kg iv + 1O ml/kg |
| f-----f----:::--- | i |
| 5 | sc. daily from Aug 20 to Sept |
| 1--:B:::-::------f----- | i 1, 201·1 |
| :::--- | |
| C1 | |
| 2̅ | AFOD: 8.3rn1/kg iv + B.3rnl/kg sc. daily from |
| | Aug 20 to Sept |
| ------------------------- | -j 1, 2011 |
| --.----------------------- | |
| . | |
| 3 | |
| J1 | |

--------------------------+----------------------
J2 1
AFOD: 6.7 ml/kg iv + 6.7rnl/kg
1------;----,----;
2 sc. daily from Aug 20 to Sept

Behavioral tests were conducted on October 8 and 9. After that, rat# A2-3, B1-2, B2-3, C1-1, C1-2, J1-1 and J2-5 were perfused for IHC staining of DA neurons. Ten negative control rats were also used for IHC staining.

4.1 Cylinder test: Since the rats were too big for cylinder test, they were not active and the number of wall contact was small, only raw data were shown here (Table 2).

TABLE 2
Number of wall contact in cylinder test

| | | | | of | | | | |
|---|---|---|---|---|---|---|---|---|
| | | | Number | contact | | | | |
| Dose | Group | No. | Left | Right | Both | Left | % Right | Both′ |
| 10 m1/iv | B1 | 2 | 4 | 1 | 0 | 80.0 | 20.0 | 0.0 |
| 4 1 O mlikg | | | | 0 | 0 | 100.0 | 0.0 | 0.0 |
| sc | 2 | 5 | 11 | 0 | 2 | 100.0 | 0.0 | 0.0 |
| | | 2 | 6 | +kg | | 75.0 | 0.0 | |
| | | | | 25.0 | | | | |
| | | 3 | 3 | 2 | 1 | 50.0 | 33.3 | 16.7 |
| 8.3 m1/kg | Cl | 1 | 12 | 3 | 10 | 60.0 | 15.0 | 25.0 |
| iv −4− | | 2 | 5 | 5 | | 25.0 | 25.0 | + |
| 8.3 rnl/kg | | | | | | | | 50.0 |
| sc | 02 | 1 | 5 | 2 | 1 | 62, 5 | 25.0 | 12.5 |
| | | 2 | 10 | 0 | 0 | 100.0 | 0.0 | 0.0 |
| | | 3 | ′ | 2 | 1 | 66.7 | 22.2 | 11.1 |
| 6.7 m1/kg | J1 | | | | | | | |
| iv+ | J2 | 1 | 1 | 0 | 0 | 100.0 | 0.0 | 0.0 |
| 6.7 mIlkg | | 2 | 0 | ., | 0 | | | |
| sc | | 4 | 0 | 0 | | | , | |
| | | 5 | 7 | 0 | 0 | 100.0 | 0.0 | 0.0 |
| control | 11 | 3 | 1 | 1 | 2 | 25.0 | 25.0 | 50.0 |
| | | 4 | | | 2 | 77.8 | 0.0 | 22.2 |
| | 12 | 2 | | | | | | |
| control | | 1 | 0 | 0 | 0 | | | |
| | | 2 | 2 | 0 | 1 | 66.7 | 0.0 | 33.3 |
| | | 3 | 12 | 1 | 1 | 85.7 | 7.1 | 7.1 |
| | | 4 | 2 | 0 | 0 | 100.0 | 0.0 | 0.0 |

4.2 Adjusting step test

All the four groups showed significant declined right forepaw step in forehand direction, furthermore, control and high dose group had significant step decline in backhand direction (FIG. 4). There was no significant difference among groups analyzed by ANOVA.

FIGS. 178A-B. Effects of daily injection of AFOD on adjusting step test. A. Forehand direction. 8. Backhand direction. Data were expressed as mean±SEM. *p<0.05.

4.3 Rotation test

Number of apomorphine induced rotation was shown in FIG. 5. All the rats had obvious rotation after injection of apomorphine. There was no significant difference among groups.

FIG. 179. Effects of daily injection of AFOD on rotation. Rats were injected s.c. with 0.25 mgikg apomorphine and rotation was counted for 5 min. Data were expressed as mean 1 SEM.

4.4 TH staining

Rats were perfused for fixation and brain sections of SN were stained with TH antibody to show dopaminergic neurons. Data were shmNn in table 3 and FIG. 6.

TABLE 3
Number of TH positive cell counting

| | | Left | | | | Neuron counting Right | | | | |
|---|---|---|---|---|---|---|---|---|---|---|
| Group | # | 1 | 2 | 3 | Sum | 1 | 2 | 3 | [symbol] | , LIR ratio |
| Control | A2-3 | 32 | 43 | 47 | 122 | 126 | 170 | 152 | [symbol] | 0.27 |
| | | | | | | | | | [symbol] | |
| | | | | | | | | | [symbol] | |
| Low | J1-1, | 15 | 24 | 24 | 63 | 97 | 101 | 123 | [symbol] | 0, 20 |
| | J2-5 | 27 | 28 | 38 | 93 | 117 | 139 | 108 | [symbol] | 0.26 |
| | | | | | | | | | [symbol] | |
| | | | | | | | | | [symbol] | |
| | | | | | | | | | [symbol] | |
| Moderate | 01-1 | 25 | 25 | 45 | 95 | 129 | 156 | 149 | 434 | 0.22 |
| | C1-2 | 74 | 45 | 85 | 204 | 169 | 182 | 221 | 572 | 0.36 |
| High | B1-2 | 91 | 63 | 111 | 265 | 141 | 133 | 179 | 453 | 0.58 |
| −i− | B2-3 | 59 | 25 | 50 | 134 | 129 | 163 | 178 | 470 | 0, 29 |
| Negative | 1 | 149 | 100 | 191 | 440 | 133 | 81 | 203 | 417 | 106 |
| | 2 | 96 | 79 | 217 | 392 | 125 | 107 | 170 | 402 | 0.98 |
| | 3 | 71 | 88 | 153 | 312 | 91 | 78 | 125 | 294 | 1, 06 |
| | 4 | 127 | 207 | 151 | 485 | 102 | 154 | 140 | 396 | 1.22 |
| | 5 | 76 | 112 | 118 | 306 | 61 | 120 | 110 | 291 | 1.05 |
| | 6 | 124 | 126 | 99 | 349 | 119 | 156 | 124 | 399 | 0, 87 |
| | . . . | 116 | 114 | 195 | 425 | 101 | 148 | 204 | 453 | 0.94 |
| | 8 | 134 | 160 | 131 | 425 | 137 | | 170 | + | 0.93 |
| | | | | | | | + | | | |
| | | | | | | | 152 | | 459 | |
| | 9 | 150 | 120 | 168 | 438 | 157 | 103 | 182 | 442 | 0.99 |
| | 10 | 112 | 135 | 193 | 440 | 154 | 187 | 141 | 482 | 0.91 |

[symbol]

indicates data missing or illegible when filed

FIG. 180. TH staining of the SN.

Rats were perfused and the brains were fixed for IHC study. Three sections from caudal, center and rostral part of the SN (bregma −55, −525 and -5.0 mm) of each brain were used for staining. Cell number of each side was counted and the ratio of left/right was calculated. Data were expressed as mean 1 SEM

5. Rotation test for post groups

The rats in post groups were tested with apormorphine induced rotation on Oct. 10, 2011. The number of rotation was shown in Table 4. No further experiment was done on these rats.

TABLE 4
Number of rotation of post groups

| | | control | high | moderate | low |
|---|---|---|---|---|---|
| | rat # | E | F | G | H |
| cage 1 | 1 | 0 | 20 | 10 | 50 |
| | 2 | 30 | 4 | 11 | 0 |
| | 3 | 17 | 11 | 0 | |
| | | 0 | 16 | 11 | 14 |
| | 5 | 5 | 17 | 0 | 16 |
| cage 2 | 1 | 12 | 15 | 0 | 71 |
| | 2 | 20 | 11 | 6 | 8 |
| | 3 | 19 | 19 | 0 | 23 |
| | 4 | 16 | 0 | 10 | 11 |
| | 5 | 2 | 8 | 4 | 14 |

All the left rats were sacrificed on Nov. 22, 2011.

Conclusion:

The inventor ordered to abort the study for therapeutic as there was no statistical data to support a valid vehicle group before the surgical operation to remove the brain in order to count the neurons. The result of the cylinder test and the rotation test on the rat did not give a very convincing result for the controL However after the operation ofthe brain to count the neurons in the vehicle control, negative control and tested prophylactic group it showed the trend that using AFOD RAAS 1 reduce the damage caused by 6-OHDA lesion in the high and moderate groups to compare with the vehicle. Other studies are being conducted using 6-OHDA lethal dose in the rat

KH good healthy cells 1—Send signals to the DAMAGED, SICK, AND BAD CELLS that triggers that synthesis of good proteins that transform these cells to become GOOD healthy cells: 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being aff(cted by intra- and extracellular damaging signals.

Report Title: Antiviral efficacy of AFCC in an influenza

H1N1 infected mouse model

Report No: WX IFV02162012

Issue Date: Apr. 11, 2012

Study No:

Study Period: Feb. 16! 201:2 to Apr. 8! 201:2

Part 1 Pilot Study

Content

Summary of the report

Objective

Infection with human influenza virus (IFV) causes respiratory tract illness in human and animals including mice. Mouse model infected Intranasally with IFV H1N1 is well recognized for anti-IFV compound screening. This study is designed to evaluate in vivo anti-IFV activity of a blood-derived product AFCC from RAAS in the mouse modeiJ L \L1 1 . . . 1\ i ml t′L.i h DL9b LE1.\ U 1QS.m g Ø.JL.. tt LfLLU.\ \?

Study method

Study RAAS-201202168 was executed in the following steps:

1) Treat mice with RAAS blood product AFCC-KH.

1) Infect mice with IFV by intranasal inoculation.

2) Observe mice for 26 days.

3) Sacrifice mice in the end of the study. Result summary

Report for RAAS 20120216B L Method

Animals:

Female BALB/c mice (6-8 weeks, 17-22 g) \Nere divided into defined study groups after a visual examination and a 3 to 5-day acclimation upon arrivaL

Solution preparation:

1. Sodium Pentobarbital: Freshly dissolved in saline for injection at 8 mg/ml prior to using.

2. Test article: human plasma derived protein AFCC in sterile solutions for vein injection provided by the client

Experimental Procedure:

IFV infection and test article administration:

1. From day 1 to day 14, AFCC KH 1 is intravenously and/or intraperitoneally administrated for 14 days.

2. On day 15, mice are anesthetized by intraperitoneal injection of sodium pentobarbital (80 mg/kg). Mice are inoculated with 5×1QA3 pfu of Influenza H1N1 AiWSN/33 via the intranasal route in SFM medium.

3. From day 1 through day 40 mice are observed two times a day. Mortality and body weight are recorded daily”

4. On day 40, the experiment is terminated by sacrificing survived mice.

II. Groups and schedules:

TABLE 1
Action summary of Study WX IFV02162012

| | | | | | |
|---|---|---|---|---|---|
| | | | IFV | ,FCC, | mouse |
| Study Day | Date | Weighing | challenge | iviip | sacrifice |

| | | | |
|---|---|---|---|
| Day 1 | Feb. 16, 2012 | ′ | ., |
| Day 2 | Feb. 17, 2012 | Ni | |
| Day 3 | Feb. 18, 2012 | | |
| Day 4 | Feb. 19, 2012 | .′ | |
| Day 5 | Feb. 20, 2012 | Ni | N |
| Day 6 | Feb. 21, 2012 | | |
| Day 7 | Feb. 22, 2012 | .′ | ., |
| Day 8 | Feb. 23, 2012 | Ni | |
| Day 9 | Feb. 24, 2012 | | |
| Day 10 | Feb. 25, 2012 | .′ | |
| Day 11 | Feb. 26, 2012 | Ni | N |
| Day 12 | Feb. 27, 2012 | | |
| Day 13 | Feb. 28, 2012 | .′ | ., |
| Day 14 | Feb. 29, 2012 | Ni | |
| Day 15 | Mar. 1, 2012 | | |
| Day 16 | Mar. 2, 2012 | .′ | |
| Day 17 | Mar. 3, 2012 | Ni | |
| Day 18 | Mar. 4, 2012 | | |
| Day 19 | Mar. 5, 2012 | .′ | |
| Day 20 | Mar. 6, 2012 | Ni | |
| Day 21 | Mar. 7, 2012 | | |
| Day 22 | Mar. 8, 2012 | .′ | |
| Day 23 | Mar. 9, 2012 | Ni | |
| Day 24 | Mar. 10, 2012 | .1 | |
| Day 25 | Mar. 11, 2012, | , | |
| | | , | |
| Day 26 | Mar. 12, 2012 | ,I | |
| Day 27 | Mar. 13, 2012 | .1 | |
| Day 28 | + | , | |
| | Mar. 14, 2012 | , | |
| Day 29 | Mar. 15, 2012 | ,I | |
| Day 30 | Mar. 16, 2012 | .1 | |
| Day 31 | + | , | |
| | Mar. 17, 2012 | , | |
| Day 32 | Mar. 18, 2012 | ,I | |
| Day 33 | Mar. 19, 2012 | .1 | |
| Day 34 | + | , | |
| | Mar. 20, 2012 | , | |
| Day 35 | Mar. 21, 2012 | ,I | |
| Day 36 | Mar. 22, 2012 | .1 | |
| Day 37 | + | , | |
| | Mar. 23, 2012 | , | |
| Day 38 | Mar. 24, 2012 | ,I | |
| Day 39 | Mar. 25, 2012 | .1 | |
| Day 40 | Mar. 26, 2012 | , | |

indicates that the action was taken.

TABLE 2
Experimental Design for the pilot experiment

| Day | AFCC-Kil | animal number | (m-_/iiouse) | | H1N1 WSN |
|---|---|---|---|---|---|
| 1 | iv, | 0.2 | 5 | | |
| 3 | ip, | 0.6 | 5 | | |
| 5 | iv, | 0.2 | 5 | | |
| 7 | ip, | 0.6 | 5 | | |
| 9 | iv, | 0.2 | 5 | | |
| 11 | ip, | 0.6 | 5 | | |
| 13 | iv, | 0.2 | 5 | | |
| 15 | ip, | 0.6* | 5 | 5 | in, 5 × 10−3 |
| 17 | | | | 5 | |
| 19 | | | | 5 | |
| 21 | | | | 5 | |
| 23 | | | | 5 | |
| 25 | | | | 5 | |
| 27 | | | | 5 | |
| 29 | | | | 5 | |
| 31 | | | 5 | 2,-;- | |
| 33 | | | 5 | 2,-;- | |
| 35 | | | 5 | 2,-;- | |
| 37 | | | 5 | 2,-;- | |
| 39 | | | 5 | 2,-;- | |
| 40 | | | 5 | :-.,,-;- | |

ill Adverse Events and Tolerability of Compounds:

1. In the AFCC treatmentgroup,--t4t--.t--1-ae-t--.t-4, one mouse w;-,:,6 1, 2.012-the e--died of severe face end aeck demees on Ma /,2012 fexoerimenta de:117) due seHous fieht .e:miong mice. This mouse was eliminated for final datass-s-ceeivais.

Results and discussion

FIG. 181. Body weight changes caused with AFCC treatment in mice

TABLE 3
Effect of AFCC on mean day to death of mice infected with H1N1
A/WSN/33

| Treatment | [symbol] |
|---|---|
| [symbol] | [symbol] ** |
| [symbol] | [symbol] |

**P < 0.0i compared to the H1N1 + vehicle control [symbol]
indicates data missing or illegible when filed

FIG. 182. Efficacy of AFCC on H1N1 WSNacaused mouse death

FIG. 183. Body weight changes caused by AFCC in mice infected with H1N1 (WSN) influenza

FIG. 184. Body weight change caused with AFCC treatment in mice infected with H1N1 (WSN) influenza

FIG. 185. Body weight change caused with Vehicle treatment in mice infected with H1N1 (WSN) influenza

### APPENDIX

The experimental raw data

Dose Adminstratiou Tahl

Part 2 Efficacy Study

Content

Summary of the report

Objective

Infection with human influenza virus (IFV) causes respiratory tract illness in human and animals including mice. Mouse model lntranasally infected with IFV H1N1 is a well recognized for antiviral compound screening against IFV infection. This study is designed to evaluate the compounc! AFCC from RAAS for anti-IFV activity in the mouse model.

Study method

This study was peliormed in the following steps:

1) Infect mice with IFV by intranasal inoculation.

2) Treat the infected mice with RAAS blood products AFCC; reference compound Oseltamivir or vehicle, starting 4 h prior to IFV inoculation.

3) Sacrifice survived mice in the end of the study. Result summary

In the H1N1-challenged vehicle control group all 10 rnice died and their body weights dramatically dropped by 20 to 30% within 4-6 days post-IFV H1N1 challenge. In comparison to the vehicle group. the mice treated po/bid with Oseltamivir survived completely and their body weights dropped by <20% JLL L Lt X1 LP9. -L-a “F”>‘-l’->:t-IFV H1N1 challenge;: mLL Wit:}Etm:

.L L ?Y -LmLnt: \ iLi:: E:L:LE!ll:LtNJf} --: m- LLtt: -- r:;mtL L :gL t. These indicate that the mouse model worked successfully in current study. Treatment with 0. •15, or 0. •1 ml/mouse of AFCC significantly prolonged the infected mouse survival time by 1.9, or 1.0 days, respectively, compared with H1N1+vehicle group, although the treatment with any AFCC dose d:dn.:t••m. !L:.m: decrease (t the animal mortality rate an i•rK L.prevent Ei. mouse body weight loss caused by the IFV H1N1 infection, compared with Oseltamivir treatment The lD::l\ni ::!YL pJreatment with 0.2 ml/mouse of AFCC ,:1!--neither ““itl”rl-li l,::<:>;:-,tl:,<-prolonmxt the infected mouse survival time nor decreasej the mouse mortality rate. .:.q,F “′”: --Il!.t;i_observations suggest. %? that tile AFCC may t.“k•′)><LktLLa limited :: :>k --L:iTL \ t t LmJ J.Lit.l:LLbtl.inklt !LLlLLlH--<:>;:-,tl--H:+N-′l--,lF\Lin the current study.

Report for RAASM20120216B I. Method

Animals:

Female BALB/c mice (6-8 weeks. 17-22 g) were divided into defined study groups after a visual examination and a 3 to 5-day acclimation upon arrivaL

Solution preparation:

1. Sodium Pentobarbital: Freshly dissolved in saline for injection at 8 rng/ml prior to using.

2. Test article: human plasma derived protein AFCC in sterile solutions for vein injection provided by the client.

3. Vehicle: PBS

4. Oseltamivir phosphate (prodrug): aqueous solution in PBS, 0.1 mg/ml

Experimental Procedure:

IFV infection and test article administration:

1. On the day of Influenza administration. mice ;*“;′”Y: ‘.L’ anesthetized by intraperitoneal injection of sodium pentobarbital (80 mg/kg).

2. Mice “′”<;′-:O::i. L-‘′’-inoculated with 5×10′″3 pfu of Influenza H1N1A/WSN/33 via the intranasal route in

SFM medium.

3. T′″>i:-,′H′i:ld“′ r_:;::or vehicle i- ′-YL; _intravenously administrated daily L L>+i:h′″ 4 days after H1N1 infection. Oseltamivir (1 mg/kg/day) c•:,:i; ; _orally given twice daily for 8 days. First dosing for oseltamivir or test article 1--)t!A -executed 4 h pre H1N1 inoculation.

4. From day 1 through day 10 the infected mice; +i″-•Y\ U c.observed two times a day. Mortality and body weight ,;H+•Y:.:-“′iLiUecorded daily.

5. On day 10, the experiment. Y}.” terminated by sacrificing survived mice.

II. Groups and schedules:

Table 4 Action summary of Study WX IFV02162012

3. On day 4 post H1N1 infection, LK Ch.t!ELt..w,:rJLLL tLLD.Jl•>k><;_H n--AFCC-0.2 rnl treatment group

0.15 ml treatment group also had hematuria. We stopped AFCC medication on the fifth day post H1N1 infection.

Results and discussion

In the H1N1-challenged vehicle control group all 10 mice died and their body weights dramatically dropped by 20 to 30% within 4-6 days post-IFV H1N1 challenge (FIG. 6, FIG. 7, and Table 4). In comparison to the vehicle group, the mice treated po/bid \Nith Oseltamivir survived cmnpletely and their body weights dropped by <20% against IFV H1N1 challenge (FIG. 6, FIG. 7, and Table 4). These indicate that the mouse model worked successfully in current study.

Treatment with 0.15, or 0.1 ml/mouse of AFCC significantly prolonged the infected mouse survival time by 1.9, or 1.0 days, respectively, compared \Nith H1N1+vehicle group (Table 4), although the treatment with any AFCC dose di<ci,ci::t.n:]lfIE..decrease.t the animal mortality rate

nnd•LE?LPrevent: 11 mouse body weight loss caused by the IFV H1N1 infection, compared with Oseltamivir treatment (FIG. 6, FIG. 7). The treatment with 0.2 rnl/mouse of AFCC: i4-neither &t:\′lf,,lf,4_-,,, , ′tly:-prolongs2_t the infected mouse survival time nor decreasej the mouse mortality rate

FIG. 186. Effect of AFCC on H1N1 g caused mouse mortality

TABLE 4
Effect of AFCC or Oseltamivir on mean day to death of mice
infected with H1N1 A/WSN/33

| | | Survivor/ | Mean day |
|---|---|---|---|
| Treatment | Dose | total | to death ± S.D. |
| H1N1 + AFCC | 0.2 ml | 0/20 | 5.1 ± 0.38 |
| | 0.15_ml | 0/10 | 7.6 ± 1.74** |
| | 0.1_rn! | 0/10 | 6.7 ± 0.9* |
| | 1 mg/kg | 10/1( ) | >10 ± 0.0** |
| | 0.2 ml | 0/1( ) | 5.7 ± 0.64 |

| | |
|---|---|
| H1N1 + Oseltamivir | |
| | --- .:;.- ----------------..r.. ------------ ; |
| | ----------------- ---- |
| H1N1 + Vehicle | ------------------------- - - -- -- --- ==. |
| | ------------------- |
| -- - - :.. ------------------- | ------ 1 j |
| -------------------- | |
| ---------------------------- | |
| ---------------------------- | |
| -- | |

*P <:0.05,
**P <:0.01 compared to the H1N1 + vehicle control

FIG. 187. The average body weight change in mice infected with H1N1 influenza

### APPENDIX

The experimental raw data for Study RAASw20120216B

Report Title: Antiviral efficacy of AFOD and AFCC in an influenza

H1N1 infected mouse model

Report No: WX-IFV01152012

Issue Date: Jan. 20, 2012

Study No: RAAS-201110170

Study Period: Jan. 1, 2012 to Jan. 15, 2012

Summary of the report

Objective

Infection with human influenza virus (IFV) causes respiratory tract illness in human and animals including mice. Mouse model lntranasally infected with IFV H1N1 is a well recognized for antiviral compound screening against IFV infection. This study is designed to evaluate the compounds AFOD and AFCC from RAAS for anti-IFV activity in the mouse model.

Study method

Study RAAS-201110170 was peliormed in the following steps:

1) Infect mice with IFV by intranasal inoculation.

2) Treat the infected mice with RAAS blood products AFOD or AFCC, reference compound Oseltamivir or vehicle, starting 4 h prior to IFV inoculation.

3) Dissect mice for organ observations by an immunologist in the end of the study. Result summary

In the H1N1-challenged vehicle control group all 10 mice died and their body weights dramatically dropped by 20 to 30% within 4-7 days post-IFV H1N1 challenge. In comparison to the vehicle group, the mice treated po/bid with Oseltamivir survived completely and their body weights dropped by <“101o against IFV H1N•1 challenge. These indicate that the mouse model worked successfully in the current study. Treatment with 0.8, or 1.2 ml/mouse of AFCC significantly prolonged the infected mouse survival time by 1.8, or 2.1 days, respectively, although the treatment with any AFCC dose didn't decrease the animal mortality rate, compared with the Oseltamivir treatment. The treatment with 1.0 ml/mouse of AFCC and with 0.8, 1.0 and 1.2 ml/mouse of AFOD did neither significantly prolong the infected mouse survival time nor decrease the mouse mortality rate.

In comparison to the vehicle group, spleens and lymph nodes of the mice in AFCC treatment group showed significantly swollen and enlargement In addition, significant intumescence and hemorrhage of mouse healis and lungs occurred in the AFOD and AFCC groups, compared with unchallenged vehicle group (photos of the organs included in the following straight matter).

Report for RAASw201110170

L Method

Animals:

Female BALB/c mice (6-8 weeks, 17-22 g) were divided into defined study groups after a visual examination and a 3 to 5-day acclimation upon arrival.

Solution preparation:

1. Vehicle: 0.9% saline

2. Ose!tarnivir phosphate (prodrug): aqueous solution in PBS, 3 mg/rnl

3. Sodium Pentobarbital: Freshly dissolved in saline for injection at 8 mg/ml prior to using.

4. Test article: human plasma derived proteins AFOD and AFCC in sterile solutions for vein injection provided by the client

Experimental Procedure:

IFV infection and test article administration:

1. On the day of IFV challenge, mice \Nere anesthetized by intraperitoneal injection of sodium pentobarbital (80 mg/kg).

2. Mice were intranasally inoculated with 5×10″3 pfu of Influenza H1N1 A/WSN/33 in SFM medium.

3. Test articles AFOD or AFCC or vehicle was iv/ip administrated every other day for first 4 days. every third day for days 5 to 7 and was suspended for dosing from days 8 to 14 following the client instructions. The reference compound Oseltamivir (30 mg/kg/day) was orally given tbid for first 8 days of the study. First dosing for the test articles or oseltamivir was executed 4 h pre

WSN H1N1 challenge.

4. From day 1 through day14 the infected mice were observed two times daily. Mortality and body weight were recorded daily.

5. On day 14, the experiment was terminated by sacrificing survivors. Mice were dissected for organs observation by an immunologist invited from WX NPII Department.

II. Groups and schedules:

TABLE 1
Action summary of Study WX IFV01152012

Study
Date
Weighi
!FV chal! enge,
AFOD/AFCC,
10:00-
7:40-
mouse sacrifice and organ
Day
ng 2:00-4:00
iv!ip,
10:00-10:20
am
8:00pm
dissection,
2:00-4:00 pm
pm 12:00 am
Day 0 01012012 ,, ′ - -.,; If i
i
Day 1
01022012 \j
Day 3 01042012 ,, -.,; If i
i
Day4
01052012 \j
Day 6 01072012 ,, -.,; If i
i
Day 7
01082012 \j
Day9 01102012 ,, i
i
Day 10
01112012 \j
Day12 01132012 ,,
Day 13
01142012 \j
Day 14 \j \j

TABLE 2
Experimental regimen for day 0 to day 5

| | | | | | |
|---|---|---|---|---|---|
| 3 | 10 | H1N1 + AFOD | 0.2 | | iv, every third day |
| | | | rnlirnouse | | |
| 4 | 10 | H1N1 + AFOD | 0.3 | | iv, every third |
| | | | rnlirnouse | | day iv, |
| 5 | ′10 | H1N1 + AFCC | ( ).′1 | | every third day |
| | | | ml/rnouse | | |
| 6 | 10 | H1N1 + AFCC | 0.2 ml/ | | iv, every third day |
| | | | rnouse | | |
| 7 | 10 | H1N1 + AFCC | 0.3 | | iv, every third day |
| | | | milmouse | | |
| 8 | 10 | H·lN·l + Oseitarnivir | 30 mg/kg/ | 10 | p.o, BID |
| | | | day | | |
| 9 | 6 | ve ;icle | 0.3 ml/ | | iv, every third day |
| | | | mouse | | |

ill Adverse Events and Tolerability of Compounds:

2. In the HiN1+1.2 mlimouse AFOD treatment group, 1 mouse died during anesthesia and IFV infection on Jan. 1, 20•12. This mouse was eliminated for final data process.

3. In the H1N•1+0.8 ml/mouse AFCC treatment group, 2 mice died after IV dosing on Jan. 3, 2012. These 2 mice were eliminated for final data analysis.

Results and discussion

In the H1N1-challenged vehicle control group all 10 mice died and their body weights dramatically dropped by 20 to 30% within 4-7 days post-IFV H1N1 challenge (FIG. 1, FIG. 2, FIG. 3, FIG. 4, Table 4). In comparison to the vehicle group, the mice treated po/bid with Oseltamivir survived completely and their body weights dropped by <10% against IFV H1N1 challenge (FIG. 1, FIG. 2, FIG. 3, FIG. 4, Table 4). These indicate that the mouse model worked successfully in current study. Treatment with 0.8, or 1.2 ml/mouse of AFCC significantly prolonged the infected mouse survival time by i 0.8, or 2.1 days, respectively, compared with HiN1+vehicle group (Table 4), although the treatment with any AFCC dose didn't decrease the animal mortality rate and prevent mouse body weight loss caused by the IFV H1N1 infection, compared with Oseltamivir treatment (FIG. 1, FIG. 3, FIG. 4). The treatment with 1.0 ml/mouse of AFCC and with 0.8, 1.0 and 1.2 ml/mouse of AFOD did neither significantly prolong the infected mouse survival time nor decrease the mouse mortality rate (FIG. 1, FIG. 2, FIG. 3, FIG. 4, Table 4). These observations suggest that the AFCC but not AFOD may play a limited role in anti-H•1 Ni IFV in the current study.

We didn't really know the toxicity data of the human plasma derived products AFOD and AFCC in both in vitro and in vivo experiments before we started this study although it was said that the products had no toxicity because they are from human blood. It is possible that the doses of AFOD and AFCC that were taken in the first 5 days in the study were beyond mouse tolerance due to in vivo toxicity including hyper-immune reaction. Indeed, in the apparent inspection of the

mouse organs in the study swollen and enlarged spleens and lymph notes were observed in the AFCC treatment group, suggesting that those mice had experienced certain toxicity probably owing to overdoses of the test article.

Taken all above together it is worth to suggest that in any future confirmative study for the anti-influenza efficacy of AFCC and AFOD, a maximum tolerated or lower dose of either the plasma derived product should be used to decrease their potential in vivo toxicities and appropriately H1N1(WSN} influenza

FIG. 191. Body weight change caused with AFCC or Oseltamivir treatment in mice infected with H1N1(WSN) influenza

### APPENDIX 1

FIG. 192. Photos of mouse organs dissected in the end of the study RAAS 201110170

### APPENDIX 2: THE EXPERIMENTAL RAW DATA FOR STUDY RAASW201110170

HBV Study Report

Efficacy of AFOD RAAS 104® (formerly AFOD RAAS 8) in the HBV Mouse Hydrodynamic Injection Model

PROJECT CODE: RASS HBV 06012012

STUDY PERIOD: Jun. 19, 2012 to Jul. 3, 2012

### 1 Introduction

Hydrodynamic injection (HOI) is an in vivo gene delivery technology. It refers to transiently transfect the mouse liver cells with a foreign gene via tail vein injection of a large volume saline containing plasmid within a few seconds. Taking the advantage of the liver-targeting manner of hydrodynamic injection, a single hydrodynamic injection of a replication-competent HBV DNA, could result in HBV replication in mouse liver shortly. This HBV hydrodynamic injection model on immunocompetent mice is a convenient and reproducible animal model for anti-HBV compound screening in vivo, which has been successfully established in WuXi ID department.

The purpose of this study is to evaluate in vivo anti-HBV efficacy of RASS 8 using the mouse hydrodynamic injection model.

### 2 Materials and Reagents

2.1. Animal: Female BALB/c mice, age 6-8 weeks, between 18-22 g.

2.2. Test article:

Vehicle: normal saline.

Entecavir (ETV): supplied as powder by ;ft′l•H %: k fK tf;′ft . . . : .L;tffR ′:- t>j, dissolved in normal saline prior to dosing.

AFOD-RAAS 8 (RAAS 8): provided by RAAS, 25% (blood-derived proteins) solution.

2.3. Reagent:

HBV plasmid DNA: pcDNA3.1/HBV, prepared with Qiagen EndoFree Plasmid Giga Kit; QIAamp 96 DNA Kit, Qiagen 51162; Universal PCR Master Mix, ABI 4324020; HBV DIG DNA

probe, prepared by PCR DIG Probe Synthesis Kit, Roche “116360909”10; DIG Wash and Block Buffer Set, Roche 11585762001; HBsAg ELISA kit, Kehua.

### 3 Experimental Procedure

3.1 Hydrodynamic injection and compound administration

3.1.1. From day −7 to day 0, all 5 mice in group 4 were administrated i.p./i.v. with test article daily for 8

days according to Table 2.

3″ 1.2″ On day 0, all groups of mice were hydrodynamicly injected via tail vein with pcDNA3.1/HBV plasmid DNA in a volume of normal saline equal to 8% of a mouse body weight. The plasmid DNA solution for injections was prepared one day before injection and then stored in 4GC until injection”

3″ 1.3″ From day 0 to day 5, mice in groups 1-3 were weighed and treated with compounds or vehicle according to the regimen in Table 2. For groups 1 and 3, the first dosing was executed 4 hours pre HDL For groups 2, the first dosing was executed 4 hours post HDI. For group 4, the last dosing was carried out 4 hours post HOI.

3.1.4. All mice were submandibularly blec! for plasma preparation according to the design in Table 1.

3.1.5. All mice were sacrificed and c!issectec! to obtain livers (two pieces of left lobe, one piece of middle lobe and one piece of right lobe) according to the regimen in table 1. Isolated livers were snap frozen in liquid nitrogen immec!iately upon collected.

Table 1. Experimental Design for the pilot experiment

Mice CPD

Dose Vol (ml/kg) Treatment Schedule 1st treatment time Injection treatme j.ig/nt bleeding liver dissect ion mouse schedule 5 Vehicle 11 See Tab2 See Table 2 4 hrs pre-injection day 7 4 tail vein day 7 1,′,,, 2 5 RAAS 8 T e2 See Table 2 hrs post-injection HDI of days pcDNA 0.′1 3 5 ETV 10 mg/kg PO, QD*, 4 hrs days 0-4 pre-injection last dosing, 20 1, 3, 3.1 HBV, 4, 5, day 0, 7 q.d. day 5 4 5 RAAS8 See Tab2 See Table 2 4 hrs post-injection day 7 QD*: once a day; Vehicle”″: normal saline Day 1 HBsAg level, in order to detect the presence of Hepatitis B surface antigen and DNA replication has been performed using ELISA method. The results show that on day one after the injection of the HBV DNA into the mouse AFOD RAAS 104@ (formerly AFOD RAAS 8) has begin to eliminate Hepatitis virus down to the n; gative control lev; 1.

FIG. 193—Day 1 of HBsAg level

Day 3—HBsAg level, in order to detect th;presence of Hepatitis B surface antigen and DNA replication has been performed using ELISA method. The results show that on day three after the injection of the HBV DNA into the mouse AFOD RAAS 104® (formerly AFOD RAAS 8) has been completely eliminated the Hepatitis B virus. AFOD RAAS 104® contains GOOD healthy cells in which the DNA sends the signal to the DNA of the bad/damaged/infected with hepatitis B virus cell to transform the RNA of the bad damaged cell to synthesize the GOOD protein against Hepatitis B virus.

FIG. 194—Day: 1 of HBsAg level

TABLE 2
Schedule for Compound administration

HOI*, IV
0.5 ml

| | | | | | | | | | | | | |
|---|---|---|---|---|---|---|---|---|---|---|---|---|
| 2 | 3 | 4 | 5 | 6 | 7 | | | | | | | |
| No | No | No | No | No | No | No | | | | | | |
| pm | No | No | No | No | No | No | No | No | No | No | No | No |
| | No | No | | | | | | | | | | |
| IP | | | | | | | | | | | | |
| am | No | No | No | No | No | No | No | | | | | |

HOI, IV
0.2 rnl IV
0.5 rnl IP
0.2 rnl IV
0.5 ml IP

| No | No | No | | | | | | | | | | |
|---|---|---|---|---|---|---|---|---|---|---|---|---|
| _2 | _:_:±:::: I−=−2 | | | | | | | | | | | |
| : | :_:_::_{—} | | | | | | | | | | | |
| 1 | am | No | No 1 | | No | No | No 1 | No | No | IP | rnl | ml |
| ml | rnl | No | No 1 | No | | | | | | | | |
| i | i | IV | IP ! | IV | IP | i | | | | | | |
| i | I | i | I | | | | | | | | | |
| pm | No | No | No | No | No | No | No | | | | | |

HOI, 0″3 0.3

| | | | | | | |
|---|---|---|---|---|---|---|
| rnl | No | ml | No | No | No | No |
| IV | | | | | | |
| −. | .′ | | | | | |

HLW: hydrodynam; c InJeCtion

3.2 Sample analysis

3.2.1 Detect HBV DNA replication level in plasma

IP IP

3.2.1.1 Isolate DNA from 50 pi plasma using QIAamp 96 DNA Blood Kit. DNA was eluted with

120 pi ddH20.

3.2.12 Run qPCR for HBV DNA quantification.

a) Dilute HBV plasmid standard by •1 0-fold from 107 copies/!JI to 10 copies/!JI. b) Prepare qPCR mix as shown below.

| PCR reagents | Volume | Volume for 100 Reactions |
|---|---|---|
| DEPC Water | !.11JI | 1101-JI |
| Taqman Universal Master Mix(2X) | 12.5fjl | 1250fjl |
| HBV Primer Forvvard(501JM) | 0.21-JI | 201JI |
| HBV Primer Reverse(50f.JM1 | 0.21-JI | 201JI |
| HBV Probe(51JM) | 1f.JI | 1001-JI |
| Total | 15fjl | 1500fjl |

c) Add 15 pi/well PCR mix to 96-well optical reaction plates. d) Add W !JI ofthe diluted plasmid standard.

e) Transfer 10 pi of the extracted DNA to the other wells” Seal the plates with optical adhesive film. Mix and centrifuge.

f) Place the p1Ia tes 1.n to q1PCR mach. 1ne amirun the program accord.lnQ t0tile t:(.: ble blow.

To eliminate the influence of input HBV plasmid, primers and probe targeting HBV sequence which detect newly replicated HBV DNA and input HBV plasmid DNA and targeting pcDNA3.1 plasmid backbone sequence which only detect the input plasmid DNA were used to do real-time PCR, respectively”

HBV DNA quantity=DNA determined by HBV primer-DNA determined by plasmid primer.

3.2.2 Detect HBsAg level in plasma

Dilute the plasma 500 fold;

Detect HBsAg level in 50 pl diluted plasma by using HBsAg ELISA kit.

3.2.3 Detect HBV intermediate DNA level in livers

3.2.3.1 Liver DNA isolation

a) Homogenize the liver tissue with Qiagen Tissue Lyser in 10 mM Tris.HCI, 10 mM EDTA, pH7.5.

b) Spin samples. Transfer the supernatant to a new tube containing equal volume of 2× proteinase K digestion buffer. Incubate at 50° C. for 3 hours. c) Extract with phenol: choroform: Isoamyl alcohol.

d) Transfer the upper phase to new tubes, add RNase A and incubate at 37° C. for 0 min.

e) Extract with phenol: choroform: Isoamyl alcohol.

f) Transfer the upper phase to new microfuge tubes, add 0.7-1 volume of isopropanol, add GlycoBiue Coprecipitant to 50 !Jg/mL, incubate at −20° C. for 30 min.

g) Centrifuge (′12000 g, 10 min) to precipitate DNA.

h) Wash the precipitate with 70°/o ethanol. Dissolve it in 25 !JI ddH20. Store DNA at −20″C until use.

3.2.3.2 qPCR for HBV DNA quantification with total liver DNA.

The total liver DNA was diluted to 10 ng/pl. Use 10 iJI diluted sample to run real-time PCR. HBV DNA quantity=DNA determined by HBV primer-DNA determined by plasmid primer.

:3.2.3.3 Southern blot to detect HBV intermediate DNA level in livers.

a) Load 50 pg DNA for each sample. Run •1.2% agarose gel in 1×TAE.

b) After denaturing the gel with 0.25 M HCI at RT, neutralize the gel with neutralizing buffer.

c) Transfer the DNA form the gel to a pre-wet positively charged nylon membrane by upward capillaty transfer overnight.

d) Remove the nylon membrane from the gel transfer assembly, UV cross--link the membrane (700 Microjoules/crr?), then wash it in 2×SSC for 5 min. Place the membrane at RT until dry.

e) Prehybridize membrane for 1 hour with hybridization buffer.

f) Pour off hybridization solution, and add the hybridization/pre-heated probe mixture, overnight

g) After hybridization and stringency washes, rinse membrane briefly in washing buffer. h) Incubate the membrane in blocking solution, then in Antibody solution.

i) After wash in washing buffer, equilibrate in Detection buffer.

j) Place membrane with DNA side facing up on a development folder (or hybridization bag) and apply COP-Star, until the membrane is evenly soaked. Immediately cover the membrane with the second sheet of the folder to spread the substrate evenly and without air bubbles over the membrane.

k) Squeeze out excess liquid and seal the edges of the development folder. Expose to X-ray film.

I) Expose to X-ray film at ′15-25” C.

### 4 Results and Discussion

To investigate the effect of tested compounds on HBV replication in hydrodynamic model, the level of HBV DNA in plasma was analyzed by real-time PCR method (FIG. 1). Because the injected HBV plasmid DNA can also be detected by the primers targeting to HBV sequence, the primers and probe targeting the backbone sequence of pcDNA3.1 vector were designed and usee! for real-time PCR to eliminate the influence of residual plasmic! in blood. The HBV quantity was calculated by the quantity determined by primers targeting HBV sequence subtracted by quantity determined by primers targeting the plasmic! backbone sequence.

The results indicated that RASS 8 significantly inhibited the HBV replication by therapeutic or prophylactic treatment in a time-dependent manner post HOI. On day 1, RASS 8 therapeutic treatment showed 23% inhibition and RASS 8 prophylactic treatment showed 37% inhibition to HBV replication. On day 3 and day 4, the inhibition percentage to HBV replication by RASS 8 therapeutic, or prophylactic treatment was >99%, which is statistically significant. On day 5, RASS 8 therapeutic treatment caused 93% inhibition while its prophylactic treatment made almost 100% inhibition. The HBV level in both RN\S 8 prophylactic and therapeutic groups recovered a little on day 7 compared to the data on day 5. As a reference compounc! for the HBV HOI model, entecavir had significant inhibition to the HBV replication in the therapeutically-

treated mice from day 3 post HOI to the end of experiment.

FIG. 195. Efficacy of therapeutic treatment or prophylactic treatment of RAAS 8 or ETV on in vivo HBV replication in HBV mouse HDi modeL The total DNA was isolated from plasma by QIAamp 96 DNA Blood Kit. The HBV viral load in plasma during the course of the experiment was quantified by real-time PCR. Data is expressed as mean±SE. * P<0.05, ** P<0.01 by Student's Hest.

Secreted HBV surface proteins are also important index for HBV replication. HBsAg level in plasma was

detected by ELISA method (FIG. 2). Both RASS 8 therapeutic and prophylactic treatment had a significant inhibitory effect on HBsAg level in plasma within 5 days post HBV HOT while ETV didn't have significant inhibition to the HBsAg generation, suggesting that the in vivo effect of RAAS 8 on the in vivo HBV replication may be through a different mechanism from the entecavir.

FIG. 196. Effect of prophylactic treatment or therapeutic treatment of RAAS 8 or ETV on the HBsAg in mouse blood. The HBsAg !eve! in plasma during the course of the experiment was determined by HBsAg ELISA kit. Data is expressed as mean±SE. * P<0.05, ** P<0.01 by Student's t-test.

Hepatitis B virus is a member of the hepadnavirus family, which replicates in livers and depends on liver specific factors. Thus, the existence of intermediate DNA in livers is a direct evidence

for HBV replication in livers. To quantify the intermediate HBV DNA in livers, the total DNA was isolated from liver and HBV DNA level was determined by real-time PCR (FIG. 3). ETV, as a positive control, significantly decreased the HBV intermediate DNA in liver on day 5. Similar to ETV, RASS 8 prophylactic treatment had a significant inhibition on the replication of HBV intermediate DNA in livers on day 7. In comparison to the prophylactic treatment of RAAS 8, its therapeutic treatment caused significant but to less extent inhibition to the liver HBV replication by real time PCR (FIG. 3).

The HBV quantity determined by real-time PCR is total copy number of rcDNA, dsDNA and ssDNA. To separate and visualize rcDNA, dsDNA and ssDNA, southern blot was performed (FIG. 4). The major form of HBV replication intermediate DNA was ssDNA, which was consistent

with report in literatures. Due to the limitation of DIG DNA probe sensitivity, we were not able to detect rcDNA or dsDNA. ssDNA decreased dramatically after RASS 8 prophylactic treatment or ETV treatment (FIG. 4), which confirms the result by real-time PCR (FIG. 3).

FIG. 197. Effect of prophylactic treatment or therapeutic treatment of RAAS 8 or ETV on the intermediate HBV replication in the mouse livers by qPCR

FIG. 198. HBV DNA level in plasma effect of treatment or therapeutic treatent of RAAS 8 or ETV.

1\ !lice in ETV group were sacrificed on day 5 and mice in the other three groups were sacrificed on day 7 post HOI. Liver DNA was isolated and subjected to real-time PCR to quantify the level of HBV replication intermediate DNA. Data is expressed as mean±SE. **P<0.01 by Student's t-test

FIG. 199. Southern blot determination of intermediate HBV DNA in mouse livers. 50 !JQ total

DNA each was subjected to southern blot. Lane 1 is 3.2 kb fragment of HBV plasmid (100 pg).

Lane

2 and lane 19 are DNA makers. Lanes 3 to 18 are samples.

FIG. 200. The body weights of mice treated with vehicle or indicated compounds during the course of experiment

In summary, the RAAS 8 significantly inhibited HBV DNA replication by prophylactic or therapeutic treatment in the current study with the mouse HOI model. Impressively the prophylactic treatment with RAAS 8 displayed stronger inhibition to the HBV replication than its therapeutic treatment although •we need more experiment to understand this phenomenon. In this study only 5 mice were used in each group. Thus the result may need to be confinned by using more animals. In addition a well-designed mechanism study may be required to clarify how the RAAS 8 protein functions against HBV infection.

IN VIVO Study of Nude Mice with Hair Growth

In our In-Vivo study for the breast cancer of nude mouse 4-6, in the first period of the study when the mice were completely treated and the tumor had disappeared the mice grew hair on the top of the head. FACS analysis showed that AFCC treatment had the effect on the population of major cell lineages in immune system. The inventor believes that the good healthy KH cells

which were used to treat mouse 4-6 has helped to build the immune system and help the hair to grow as the nude mice has no hair.

FIG. 201

IN VIVO Pilot Study of Nerve Repair in Goat, Monkey and Rat at

Tsinghua University of Beijing

In the pilot study at the Tsinghua University of Beijing two centimeters of the goat's leg nerve have been cut and repaired by using the FibringlueRAAS® (under different patent application) in combination with the powder form of Human Albumin and lrnrnunoglobulin (process AFOD RAAS 101® and AFOD RAAS

102®). The good healthy KH cells seem to helped restore the nerve function within a few months period, in which the RNA synthesizes good proteins that: 1—Send signal to the DAMAGED, SICK, AND BAD CELLS that triggers that synthesis of good proteins that transform these cells to become GOOD healthy cells. 2—

Send signal to the other currently undamaged cells to synthesis of good proteins to protect them from being DAMAGED, INFECTED and PRONE to DNA and other cellular alterations. 3 . . . Send signal to the body to produce new cells that are healthy and forbid them from being affected by intra- and extracellular damaging signals.

The same result was observed in Rats and Monkeys. Full study for health authority application is being carried out at the Tsinghua University of Beijing.

FIGS. 202, 203, 204 & 205

FIGS. 206, 207 & 208

Peripheral nerve injury and repair cooperated with Dr Ao Qiang of 2″″d affiliated hospital to Tsinghua university

FIGS. 209, 210 & 211

FIG. 211. The goat has recovered from the nerve damage thank to the good healthy Schwann cell.

FIGS. 212, 213, 214 and 215

HEALTHY CELLS.

Process of AFOD and AFCC

Description of figures of Manufacturing of AFOD RAAS and AFCC RAAS process.

SEE BRIEF DESCRIPTION OF THE DRAWINGS

| Cryopaste protein | |
|---|---|
| analisys | |
| 1/A1 | nitric oxide synthase 1 (neuronal), isoform CRA_b |
| 2/A2 | Chain L, Crystal Structure Of Human Fibrinogen |
| 3/A3 | Chain A, Structure Of Human Serum Albumin With |
| 4/A4 | Chain A, Human Serum Albumin In A Complex With Myristic Acid |
| | And Tri- |
| | Iodobenzoic Acid |
| 5/A5 | Chain A, Structure Of Human Serum Albumin With S-Naproxen |
| | And The Ga |
| | Module |
| 6/A6 | Chain G, Crystal Structure Of Human Fibrinogen |
| 7/A7 | Chain G, Crystal Structure Of Human Fibrinogen |
| 8/A8 | Chain G, Crystal Structure Of Human Fibrinogen |
| 9/A9 | Chain G, Crystal Structure Of Human Fibrinogen |
| 10/A10 | Chain G, Crystal Structure Of Human Fibrinogen |
| 11/A11 | fibrin beta |
| 12/A12 | fibrin beta |
| 13/A13 | fibrin beta |
| 14/A14 | fibrin beta |
| 15/A15 | fibrin beta |
| 16/A16 | Chain L, Crystal Structure Of Human Fibrinogen |
| 17/A17 | Chain I, Crystal Structure Of Human Fibrinogen |
| 18/A18 | Chain I, Crystal Structure Of Human Fibrinogen |
| 19/A19 | Chain I, Crystal Structure Of Human Fibrinogen |
| 20/A20 | fibrinogen gamma |
| 21/A21 | fibrinogen gamma |
| 22/A22 | Chain L, Crystal Structure Of Human Fibrinogen |
| 23/A23 | Chain A, Crystal Structure Of A1pi-Pittsburgh In The Native |
| | Conformation |
| 24/A24 | Keratin, Thype II cytoskeletal |
| 25 | N |
| Frac. III protein | |
| analysis | |
| 26 | N |
| 27 | N |
| 28/B1 | unnamed protein product |
| 29/B2 | unnamed protein product |
| 30/B3 | vinculin, isoform CRA_a |
| 31/B4 | unnamed protein product |
| 32/B5 | unnamed protein product |
| 33/B6 | unnamed protein product |
| 34/B7 | Chain A, Crystal Structure Of Complement C3b In Complex With |
| | Factors B And D |
| 35/B8 | fibrin beta |
| 36/B9 | fibrin beta |
| 37/B10 | fibrin beta |
| 38/B11 | Chain A, Human Serum Albumin In A Complex With Myristic Acid |
| | And Tri- |
| | Iodobenzoic Acid |
| 39/B12 | unnamed protein product |
| 40/B13 | unnamed protein product |
| 41/B14 | unnamed protein product |
| 42/B15 | unnamed protein product |
| 43/B16 | unnamed protein product |
| 44/B17 | Chain I, P14-Fluorescein-N135q-S380c-Antithrombin-Iii |
| 45/B18 | Chain I, P14-Fluorescein-N135q-S380c-Antithrombin-Iii |
| 46/B19 | growth-inhibiting protein 25 |
| 47/B20 | growth-inhibiting protein 25 |
| 48/B21 | Chain L, Crystal Structure Of Human Fibrinogen |
| 49/B22 | fibrinogen gamma |
| 50/B23 | CD5 antigen-like |
| 51/B24 | apolipoprotein A-IV precursor |
| 52/C1 | Chain C, Molecular Basis For Complement Recognition |
| 53/C2 | Chain B, H-Ficolin |
| 54/C3 | complement C4-B-like isoform 2 |
| 55/C4 | immunoglobulin light chain |
| 56/C5 | Chain A, Crystal Structure Of The Fab Fragment Of A Human |
| | Monoclonal Igm |
| | Cold Agglutinin |
| 57/C6 | immunoglobulin light chain |
| 58/C7 | PR domain containing 8, isoform CRA_b |
| 59/C8 | Chain D, The Structure Of Serum Amyloid P Component |
| | Bound To |
| | Phosphoethanolamine |
| PCC protein | |
| analysis | |
| 60/C9 | unnamed protein product |
| 61/C10 | retinol binding protein 4, plasma, isoform CRA_a |
| 62/C11 | Chain A, Crystal Structure Of Transthyretin |
| | In Complex With |
| | Iododiflunisal-Betaalaoh |
| 63/C12 | unnamed protein product |
| 64/C13 | complement component 9, isoform CRA_a |
| 65/C14 | unnamed protein product |
| 66/C15 | unnamed protein product |
| 67/C16 | unnamed protein product |
| 68/C17 | unnamed protein product |
| 69/C18 | kininogen 1, isoform CRA_a |
| 70/C19 | beta-tubulin |
| 71/C20 | vimentin, isoform CRA_a |
| 72/C21 | complement component C4B |
| 73/C22 | complement component C4B |
| 74/C23 | Chain C, Molecular Basis For Complement Recognition |
| | And Inhibition |
| | Determined By Crystallographic Studies Of The Staphylococcal |
| | Complement |
| | Inhibitor (Scin) Bound To C3c And C3b |
| 75/C24 | unnamed protein product |
| 76/D1 | unnamed protein product |
| 78/D3 | Chain D, The Structure Of Serum Amyloid P Component |
| | Bound To |
| | Phosphoethanolamine |
| 79/D4 | 24-kDa subunit of Complex I |
| Fraction | |
| IV | |
| 1 | Transferrin |
| 2 | HA |
| 3 | A1AT |
| 4 | A1AT |
| 5 | vitamin D-binding protein precursor |
| 6 | Semenogelin-1 |
| 7 | Haptoglobin |
| 8 | Vimentin |
| 9 | Not identified |
| 10 | Not identified |
| 11 | Nesprin-2 |
| 12 | Not identified |
| 13 | APOAI |
| 14 | APOAI |
| 15 | Haptoglobin |
| AFCC | |
| KH | |
| 1 | C3 Complement C3 |
| 2 | ENO1 Isoform |
| 3 | ENO1 Isoform |
| 4 | TUFM elongation factor |
| 5 | ASS1 Argininosuccinate |
| 6 | ASS1 Argininosuccinate |
| 7 | ANXA2 Isoform 2 of Annexin A2 |
| 8 | Glyceraldehyde-3-phosphate dehydrogenase |
| 9 | Glyceraldehyde-3-phosphate dehydrogenase |
| 10 | Glyceraldehyde-3-phosphate dehydrogenase |
| 11 | ANXA2 Isoform 2 of Annexin A2 |
| 12 | KRT86 Keratin, type II cuticular Hb6 |
| 13 | Glyceraldehyde-3-phosphate dehydrogenase |
| 14 | Glyceraldehyde-3-phosphate dehydrogenase |
| 15 | no matched protein found |
| 16 | LDHA Isoform 1 of L-lactate dehydrogenase A chain |
| 17 | Fibrin beta |
| 18 | Unnamed protein |
| 19 | growth-inhibiting protein 25 |
| 20 | fibrinogen gamma |
| 21 | Chain L, Crystal Structure Of Human Fibrinogen |
| 22 | growth-inhibiting protein 25 |
| 23 | Chain A of IgM |
| | Chain A, Crystal Structure Of The Fab Fragment Of A Human |
| 24 | Monoclonal Igm |
| | Cold Agglutinin |
| 25 | immunoglobulin light chain |
| 26 | Chain C, Molecular Basis For Complement Recognition |
| 15 | no matched protein found |
| 16 | LDHA Isoform 1 of L-lactate dehydrogenase A chain |
| 17 | Fibrin beta |
| AFOD | |
| KH | |
| 1 | CP 98 kDa protein |
| 2 | CP Ceruloplasmin |
| 3 | KRT2 Keratin, type II cytoskeletal 2 epidermal |
| 4 | no matched protein found |
| 5 | no matched protein found |
| 6 | no matched protein found |
| 7 | no matched protein found |
| 8 | APOA1 Apolipoprotein A-I |
| 9 | APOA1 Apolipoprotein A-I |
| 10 | APOA1 Apolipoprotein A-I |
| 11 | APOA1 Apolipoprotein A-I |
| 12 | Human albumin |
| 13 | Transferrin |
| 14 | Vimentin |
| 15 | Haptoglobin |
| APO | |
| AI | |
| 1 | APOAI |
| 2 | APOAI |
| 3 | APOAI |

FIG. 216—FR III, APCC KH

FIG. 217 APCC KH

1 C3 Complement C3

Complement component 3, often simply called C3, is a protein of the immune system. It plays a central role in the complement system and contributes to innate immunity.C3 plays a central role in the activation of complement system.[3] Its activation is required for both classical and alternative complement activation pathways. People with C3 deficiency are susceptible to bacterial infection.

2 ENO1 Isoform

ENO1 is a homodimeric soluble protein that encodes a smaller monomeric structural lens protein, tau-crystallin. ENO1 is a glycolytic enzyme expressed in mainly all tissues. ENO1 isoenzyme full length protein is found in the cytoplasm. The shorter protein is formed from another translation start that is restricted to the nucleus, and binds to a component in the c-myc promoter. ENO1 is involved in anaerobic metabolism under hypoxic conditions and plays a role as a cell surface plasminogen receptor during tissue invasion. Irregular expression of Enolase-1 is linked with tumor progression in several cases of breast and lung cancer. Enolase-1 is as an auto antigen associated with Hashimoto's encephalopathy and severe asthma. ENO1 is the target protein of serum anti-endothelial antibody in Behcet's disease.

3 ENO1 Isoform

See above

4 TUFM elongation factor

Defects in TUFM are the cause of combined oxidative phosphorylation deficiency type 4 (COXPD4). COXPD4 is characterized by neonatal lactic acidosis, rapidly progressive encephalopathy, severely decreased mitochondrial protein synthesis, and combined deficiency of mtDNA-related mitochondrial respiratory chain complexes.

5 ASS1 Argininosuccinate

The ASS1 gene provides instructions for making an enzyme called argininosuccinate synthase 1. This enzyme participates in the urea cycle, which is a sequence of chemical reactions that takes place in liver cells. The urea cycle processes excess nitrogen that is generated as the body uses proteins. The excess nitrogen is used to make a compound called urea, which is excreted from the body in urine. Argininosuccinate synthase 1 is responsible for the third step of the urea cycle. This step combines two protein building blocks (amino acids), citrulline and aspartate, to form a molecule called argininosuccinic acid. A series of additional chemical reactions uses argininosuccinic acid to form urea.

At least 50 mutations that cause type I citrullinemia have been identified in the ASS1 gene. Most of these mutations change single amino acids in the argininosuccinate synthase 1 enzyme. These genetic changes likely alter the structure of the enzyme, impairing its ability to bind to molecules such as citrulline and aspartate. A few mutations lead to the production of an abnormally short version of the enzyme that cannot effectively play its role in the urea cycle.

Defects in argininosuccinate synthase 1 disrupt the third step of the urea cycle, preventing the liver from processing excess nitrogen into urea. As a result, nitrogen (in the form of ammonia) and other byproducts of the urea cycle (such as citrulline) build up in the bloodstream. Ammonia is toxic, particularly to the nervous system. An accumulation of ammonia during the first few days of life leads to poor feeding, vomiting, seizures, and the other signs and symptoms of type I citrullinemia.

6 ASS1 Argininosuccinate

As above

7 ANXA2 Isoform 2 of Annexin A2

Annexin 2 is involved in diverse cellular processes such as cell motility (especially that of the epithelial cells), linkage of membrane-associated protein complexes to the actin cytoskeleton, endocytosis, fibrinolysis, ion channel formation, and cell matrix interactions. It is a calcium-dependent phospholipid-binding protein whose function is to help organize exocytosis of intracellular proteins to the extracellular domain. Annexin II is a pleiotropic protein meaning that its function is dependent on place and time in the body. This protein is a member of the annexin family. Members of this calcium-dependent phospholipid-binding protein family play a role in the regulation of cellular growth and in signal transduction pathways. This protein functions as an autocrine factor which heightens osteoclast formation and bone resorption. 8 Glyceraldehyde-3-phosphate dehydrogenase

Glyceraldehyde 3-phosphate dehydrogenase (GAPDH) catalyses the conversion of glyceraldehyde 3-phosphate as the name indicates. This is the 6th step of the breakdown of glucose (glycolysis), an important pathway of energy and carbon molecule supply located in the cytosol of eukaryotic cells. Glyceraldehyde 3-phosphate is converted to D-glycerate 1,3-bisphosphate in two coupled steps. The first is favourable and allows the second unfavourable step to occur.

Testis-specific: May play an important role in regulating the switch between different pathways for energy production during spermiogenesis and in the spermatozoon. Required for sperm motility and male fertility

9 Glyceraldehyde-3-phosphate dehydrogenase

As above

10 Glyceraldehyde-3-phosphate dehydrogenase

As above

11 ANXA2 Isoform 2 of Annexin A2

Please refer to Nr 7

12 KRT86 Keratin, type II cuticular Hb6

Keratin, type II cuticular Hb6 is a protein that in humans is encoded by the KRT86 gene.

The protein encoded by this gene is a member of the keratin gene family. As a type II hair keratin, it is a basic protein which heterodimerizes with type I keratins to form hair and nails. The type II hair keratins are clustered in a region of chromosome 12q13 and are grouped into two distinct subfamilies based on structure similarity. One subfamily, consisting of KRTHB1, KRTHB3, and KRTHB6, is highly related. The other less-related subfamily includes KRTHB2, KRTHB4, and KRTHB5. All hair keratins are expressed in the hair follicle; this hair keratin, as well as KRTHB1 and KRTHB3, is found primarily in the hair cortex. Mutations in this gene and KRTHB1 have been observed in patients with a rare dominant hair disease, monilethrix.

13 Glyceraldehyde-3-phosphate dehydrogenase

Please Refer to Nr 8

14 Glyceraldehyde-3-phosphate dehydrogenase

Please Refer to Nr 8

15 KH1 Protein—No matched protein found, now named KH1 Protein

IPI0089369

9

Peptide Information

Tax_Id=9606 Gene_Symbol=C1D 20 kDa protein

| | | | | Start | End | |
|---|---|---|---|---|---|---|
| Calc. Mass | Obsrv. Mass | ±da | ±ppm | Seq. | Seq. | Sequence |
| 811.3801 | 811.4196 | 0.0395 | 49 | 73 | 79 | TMMSVSR |
| 827.3749 | 827.4133 | 0.0384 | 46 | 73 | 79 | TMMSVSR |
| 831.4683 | 831.4334 | -0.0349 | -42 | 168 | 175 | NASKVANK |
| 835.4785 | 835.4451 | -0.0334 | -40 | 151 | 158 | GAASRFVK |
| 857.4515 | 857.4796 | 0.0281 | 33 | 159 | 165 | NALWEPK |
| 913.4989 | 913.5715 | 0.0726 | 79 | 86 | 93 | LDPLEQAK |
| 913.4989 | 913.5715 | 0.0726 | 79 | 86 | 93 | LDPLEQAK |
| 1231.6833 | 1231.7903 | 0.107 | 87 | 156 | 165 | FVKNALWEPK |
| IPI00218730 | | | | | | |

Peptide Information

Tax_Id = 9606 Gene_Symbol = PDE6A Rod
cGMP-specific 3′,5′-cyclic phosphodiesterase subunit
alpha

| | | | | Start | End | |
|---|---|---|---|---|---|---|
| Calc. Mass | Obsrv. Mass | ±da | ±ppm | Seq. | Seq. | Sequence |
| 813.4141 | 813.4094 | −0.0047 | −6 | 312 | 317 | EINFYK |
| 817.4526 | 817.4706 | 0.018 | 22 | 828 | 834 | QKQQSAK |
| 817.4526 | 817.4706 | 0.018 | 22 | 828 | 834 | QKQQSAK |
| 826.424 | 826.3994 | −0.0246 | −30 | 678 | 683 | RTMFQK |
| 826.424 | 826.3994 | −0.0246 | −30 | 678 | 683 | RTMFQK |
| 859.4771 | 859.498 | 0.0209 | 24 | 207 | 213 | DEEILLK |
| 867.4471 | 867.4705 | 0.0234 | 27 | 630 | 636 | HHLEFGK |
| 891.4604 | 891.451 | −0.0094 | −11 | 820 | 826 | MKVQEEK |
| 895.4091 | 895.3915 | −0.0176 | −20 | 268 | 274 | AFLNCDR |
| 963.4426 | 963.4947 | 0.0521 | 54 | 94 | 100 | MSLFMYR |
| 1209.674 | 1209.7273 | 0.0533 | 44 | 535 | 544 | FHIPQEALVR |
| 1320.614 | 1320.6102 | −0.0038 | −3 | 373 | 383 | EPLDESGWMIK |
| 1350.7642 | 1350.7184 | −0.0458 | −34 | 630 | 640 | HHLEFGKTLLR |
| 1350.7642 | 1350.7184 | −0.0458 | −34 | 630 | 640 | HHLEFGKTLLR |
| 1852.0004 | 1852.0034 | 0.003 | 2 | 312 | 326 | EINFYKVIDYI |
| | | | | | | LHGK |

Peptide Information

Tax_Id = 9606 Gene_Symbol = PLCH2 Isoform 3 of
1-phosphatidylinositol-4,5-bisphosphate phosphodiesterase eta-2

| Calc. Mass | Obsrv. Mass | ±da | ±ppm | Start | End | Sequence |
|---|---|---|---|---|---|---|
| Calc. Mass | Obsrv. Mass | ±da | ±ppm | Seq. | Seq. | Sequence |
| 808.4498 | 808.402 | −0.0478 | −59 | 210 | 215 | VKQMFR |
| 809.3458 | 809.4174 | 0.0716 | 88 | 739 | 745 | DSMLGDR |
| 813.3705 | 813.4094 | 0.0389 | 48 | 237 | 242 | MMSTRR |
| 817.4526 | 817.4706 | 0.018 | 22 | 513 | 519 | VENTAKR |
| 817.4526 | 817.4706 | 0.018 | 22 | 512 | 518 | RVENTAK |
| 824.4447 | 824.4203 | −0.0244 | −30 | 210 | 215 | VKQMFR |
| 827.4846 | 827.4133 | −0.0713 | −86 | 1253 | 1260 | VSGPGVRR |
| 828.4799 | 828.4109 | −0.069 | −83 | 900 | 906 | SQKPGRR |
| 831.4683 | 831.4334 | −0.0349 | −42 | 892 | 899 | RTASAPTK |
| 856.5615 | 856.5053 | −0.0562 | −66 | 852 | 859 | VKQALGLK |
| 891.4934 | 891.451 | −0.0424 | −48 | 266 | 272 | FLQVEQK |
| 915.5006 | 915.4615 | −0.0391 | −43 | 1164 | 1171 | SKSNPNLR |
| 1350.7601 | 1350.7184 | −0.0417 | −31 | 1240 | 1252 | LSHSLGLPGGTRR |
| 1350.7601 | 1350.7184 | −0.0417 | −31 | 1239 | 1251 | RLSHSLGLPGGTR |
| 1556.7261 | 1556.8342 | 0.1081 | 69 | 152 | 165 | YLMAGISDEDSLAR |

Peptide Information

Tax_Id = 9606 Gene_Symbol = PACRGL Uncharacterized
protein

| | | | | Start | End | |
|---|---|---|---|---|---|---|
| Calc. Mass | Obsrv. Mass | ±da | ±ppm | Seq. | Seq. | Sequence |
| 851.4469 | 851.4418 | −0.0051 | −6 | 24 | 31 | TSSSTQLK |
| 963.4741 | 963.4947 | 0.0206 | 21 | 4 | 13 | SEGSGGTQLK |
| 1350.6682 | 1350.7184 | 0.0502 | 37 | 1 | 13 | MQKSEGSGGTQLK |
| 1350.6682 | 1350.7184 | 0.0502 | 37 | 1 | 13 | MQKSEGSGGTQLK |
| 2283.1768 | 2283.4019 | 0.2251 | 99 | 66 | 86 | TINPFGEQSRVPSA |
| | | | | | | FA |
| | | | | | | AT |
| | | | | | | YSK |

Peptide Information

Tax_Id = 9606 Gene_Symbol = PACRGL
Uncharacterized protein

| | | | | Start | End | |
|---|---|---|---|---|---|---|
| Calc. Mass | Obsrv. Mass | ±da | ±ppm | Seq. | Seq. | Sequence |
| 851.4469 | 851.4418 | −0.0051 | −6 | 24 | 31 | TSSSTQLK |
| 963.4741 | 963.4947 | 0.0206 | 21 | 4 | 13 | SEGSGGTQLK |
| 1350.6682 | 1350.7184 | 0.0502 | 37 | 1 | 13 | MQKSEGSGGTQ |
| | | | | | | LK |
| 1350.6682 | 1350.7184 | 0.0502 | 37 | 1 | 13 | MQKSEGSGGTQLK |
| 2283.1768 | 2283.4019 | 0.2251 | 99 | 66 | 86 | TINPFGEQSRVPSAF |
| | | | | | | AT |
| | | | | | | YSK |

Peptide Information

Tax_Id = 9606 Gene_Symbol = PLCH2 Isoform 1 of
1-phosphatidylinositol-4,5-bisphosphate phosphodiesterase eta-2

| | | | | Start | End | |
|---|---|---|---|---|---|---|
| Calc. Mass | Obsrv. Mass | ±da | ±ppm | Seq. | Seq. | Sequence |
| 808.4498 | 808.402 | −0.0478 | −59 | 210 | 215 | VKQMFR |
| 809.3458 | 809.4174 | 0.0716 | 88 | 775 | 781 | DSMLGDR |
| 813.3705 | 813.4094 | 0.0389 | 48 | 237 | 242 | MMSTRR |
| 817.4526 | 817.4706 | 0.018 | 22 | 513 | 519 | VENTAKR |
| 817.4526 | 817.4706 | 0.018 | 22 | 512 | 518 | RVENTAK |
| 824.4447 | 824.4203 | −0.0244 | −30 | 210 | 215 | VKQMFR |
| 827.4846 | 827.4133 | −0.0713 | −86 | 1289 | 1296 | VSGPGVRR |
| 828.4799 | 828.4109 | −0.069 | −83 | 936 | 942 | SQKPGRR |
| 831.4683 | 831.4334 | −0.0349 | −42 | 928 | 935 | RTASAPTK |
| 856.5615 | 856.5053 | −0.0562 | −66 | 888 | 895 | VKQALGLK |
| 891.4934 | 891.451 | −0.0424 | −48 | 266 | 272 | FLQVEQK |
| 915.5006 | 915.4615 | −0.0391 | −43 | 1200 | 1207 | SKSNPNLR |
| 1350.7601 | 1350.7184 | −0.0417 | −31 | 1276 | 1288 | LSHSLGLPGGTRR |
| 1350.7601 | 1350.7184 | −0.0417 | −31 | 1275 | 1287 | RLSHSLGLPGGTR |
| 1556.7261 | 1556.8342 | 0.1081 | 69 | 152 | 165 | YLMAGISDEDSLAR |

Peptide Information

| | | | | Start | End | |
|---|---|---|---|---|---|---|
| Calc. Mass | Obsrv. Mass | ±da | ±ppm | Seq. | Seq. | Sequence |
| 825.4287 | 825.4099 | −0.0188 | −23 | 9 | 14 | TIYLCR |
| 837.4061 | 837.4302 | 0.0241 | 29 | 180 | 187 | SSADSSRK |
| 851.4985 | 851.4418 | −0.0567 | −67 | 38 | 45 | KFASALSK |
| 861.4828 | 861.4011 | −0.0817 | −95 | 57 | 63 | VWTSQLK |
| 963.4934 | 963.4947 | 0.0013 | 1 | 74 | 80 | LPYEQWK |
| 1245.6951 | 1245.7703 | 0.0752 | 60 | 54 | 63 | DLRVWTSQLK |

Peptide Information

Tax_Id = 9606 Gene_Symbol = NOTO Homeobox
protein notochord

| Calc. | Obsrv. | | | Start | End | |
|---|---|---|---|---|---|---|
| Mass | Mass | ±da | ±ppm | Seq. | Seq. | Sequence |
| 822.4468 | 822.3993 | −0.0475 | −58 | 211 | 216 | YQKQQK |
| 827.4482 | 827.4133 | −0.0349 | −42 | 24 | 31 | SGRSPAPR |
| 849.4366 | 849.4159 | −0.0207 | −24 | 202 | 207 | VWFQNR |
| 856.4457 | 856.5053 | 0.0596 | 70 | 1 | 7 | MPSPRPR |
| 859.4632 | 859.498 | 0.0348 | 40 | 195 | 201 | LTENQVR |
| 870.5519 | 870.5197 | −0.0322 | −37 | 187 | 194 | AQLAARLK |
| 870.5519 | 870.5197 | −0.0322 | −37 | 187 | 194 | AQLAARLK |

Peptide

Information

| Calc. | Obsrv. | | | Start | End | |
|---|---|---|---|---|---|---|
| Mass | Mass | ±da | ±ppm | Seq. | Seq. | Sequence |
| 817.4162 | 817.4706 | 0.0544 | 67 | 90 | 95 | ELDRER |
| 817.4162 | 817.4706 | 0.0544 | 67 | 90 | 95 | ELDRER |
| 826.3724 | 826.3994 | 0.027 | 33 | 1 | 8 | MGGTTSTR |
| 826.3724 | 826.3994 | 0.027 | 33 | 1 | 8 | MGGTTSTR |
| 835.438 | 835.4451 | 0.0071 | 8 | 2 | 9 | GGTTSTRR |
| 848.4108 | 848.3859 | −0.0249 | −29 | 113 | 119 | SEEERAK |
| 859.4631 | 859.498 | 0.0349 | 41 | 87 | 93 | QAKELDR |

16 LDHA Isoform 1 of L-lactate dehydrogenase A chain

Lactate dehydrogenase catalyzes the interconversion of pyruvate and lactate with concomitant interconversion of NADH and NAD+. It converts pyruvate, the final product of glycolysis, to lactate when oxygen is absent or in short supply, and it performs the reverse reaction during the Cori cycle in the liver. At high concentrations of lactate, the enzyme exhibits feedback inhibition, and the rate of conversion of pyruvate to lactate is decreased.

It also catalyzes the dehydrogenation of 2-Hydroxybutyrate, but it is a much poorer substrate than lactate. There is little to no activity with beta-hydroxybutyrate.

17 Fibrin beta

Fibrin (also called Factor Ia) is a fibrous, non-globular protein involved in the clotting of blood. It is a fibrillar protein that is polymerised to form a “mesh” that forms a hemostatic plug or clot (in conjunction with platelets) over a wound site.

18 KH2 Protein—No matched protein found, now named KH2 Protein

IPI0089369

3

Peptide Information

Tax_Id = 9606 Gene_Symbol = CCDC88A 137 kDa
protein

| | | | | Start | End | |
|---|---|---|---|---|---|---|
| Calc. Mass | Obsrv. Mass | ±da | ±ppm | Seq. | Seq. | Sequence |
| 879.4968 | 879.4153 | −0.0815 | −93 | 635 | 642 | KSSMVALK |
| 880.5138 | 880.4396 | −0.0742 | −84 | 129 | 135 | YKLLESK |
| 896.4584 | 896.4399 | −0.0185 | −21 | 365 | 371 | NLEVEHR |
| 985.5789 | 985.582 | 0.0031 | 3 | 16 | 23 | LRQQAEIK |
| 985.5789 | 985.582 | 0.0031 | 3 | 16 | 23 | LRQQAEIK |
| 1021.5272 | 1021.5333 | 0.0061 | 6 | 961 | 969 | ESSLSRQSK |
| 1021.5425 | 1021.5333 | −0.0092 | −9 | 178 | 185 | NYEALKQR |
| 1187.6267 | 1187.6656 | 0.0389 | 33 | 383 | 392 | QKGQLEDLEK |
| | 1187.6656 | | | | | |
| 1187.6267 | 1187.6656 | 0.0389 | 33 | 383 | 392 | QKGQLEDLEK |
| 1199.5903 | 1199.6674 | 0.0771 | 64 | 435 | 444 | ETEVLQTDHK |
| 1254.6212 | 1254.6615 | 0.0403 | 32 | 345 | 355 | QASEYESLISK |
| 1406.7274 | 1406.6833 | −0.0441 | −31 | 372 | 382 | DLEDRYNQLLK |
| 1406.7274 | 1406.6833 | −0.0441 | −31 | 372 | 382 | DLEDRYNQLLK |
| 1428.6754 | 1428.7153 | 0.0399 | 28 | 909 | 921 | SVSGKTPGDFYDR |
| 1479.6996 | 1479.7794 | 0.0798 | 54 | 875 | 887 | SSSQENLLDEVMK |
| 1502.8425 | 1502.8582 | 0.0157 | 10 | 78 | 90 | TLVTLREDLVSEK |
| 1727.9286 | 1727.8947 | −0.0339 | −20 | 223 | 237 | LIEVERNNATLQAEK |
| 2213.1084 | 2213.2441 | 0.1357 | 61 | 935 | 955 | KTEDTYFISSAGKPTP |
| | | | | | | GT |
| | | | | | | QGK |
| 2233.0918 | 2233.0076 | −0.0842 | −38 | 515 | 532 | TLLEQNMESKDLFHV |
| | | | | | | EQR |
| 2233.0918 | 2233.2017 | 0.1099 | 49 | 515 | 532 | TLLEQNMESKDLFHV |
| | | | | | | EQR |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3736 | 3743 | LMALGPIR |
| 870.5229 | 870.5385 | 0.0156 | 18 | 3736 | 3743 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 910.4265 | 910.4448 | 0.0183 | 20 | 3476 | 3482 | YSEIQDR |
| 910.4265 | 910.4448 | 0.0183 | 20 | 3476 | 3482 | YSEIQDR |
| 928.4669 | 928.4629 | −0.004 | −4 | 3652 | 3658 | KEVMEHR |
| 1021.5499 | 1021.5333 | −0.0166 | −16 | 2551 | 2558 | QQVQFMLK |
| 1021.5499 | 1021.5333 | −0.0166 | −16 | 2551 | 2558 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3312 | 3321 | VVKAQIQEQK |
| 1187.6201 | 1187.6656 | 0.0455 | 38 | 2757 | 2766 | NCPISAKLER |
| 1187.6201 | 1187.6656 | 0.0455 | 38 | 2757 | 2766 | NCPISAKLER |
| 1199.6896 | 1199.6674 | −0.0222 | −19 | 3758 | 3767 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4662 | 4672 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4647 | 4658 | QPVYDTTIRTGR |
| 1406.7386 | 1406.6833 | −0.0553 | −39 | 4647 | 4658 | QPVYDTTIRTGR |
| 1413.7809 | 1413.8057 | 0.0248 | 18 | 3156 | 3167 | ARQEQLELTLGR |
| 1420.7213 | 1420.6881 | −0.0332 | −23 | 2940 | 2951 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 5052 | 5063 | LNDALDRLEELK |
| 1465.7281 | 1465.7726 | 0.0445 | 30 | 4428 | 4439 | EETYNQLLDKGR |
| 1465.7316 | 1465.7726 | 0.041 | 28 | 4440 | 4453 | LMLLSRDDSGSGSK |
| 1487.7952 | 1487.7654 | −0.0298 | −20 | 3565 | 3577 | QTTGEEVLLIQEK |
| 1502.873 | 1502.8582 | −0.0148 | −10 | 380 | 391 | LLEVWIEFGRIK |
| 1532.6785 | 1532.7728 | 0.0943 | 62 | 3891 | 3903 | ELNPEEGEMVEEK |
| 1713.8728 | 1713.8539 | −0.0189 | −11 | 3123 | 3137 | HMLEEEGTLDLLGLK |
| 1727.9149 | 1727.8947 | −0.0202 | −12 | 2151 | 2165 | KLLPQAEMFEHLSGK |
| 1794.9636 | 1794.8103 | −0.1533 | −85 | 5106 | 5121 | QEFIDGILASKFPTTK |
| 1838.8412 | 1838.927 | 0.0858 | 47 | 4960 | 4974 | ALIAEHQTFMEEMTR |
| 2186.155 | 2185.9851 | −0.1699 | −78 | 1958 | 1978 | LLSDTVASDPGVLQE |
| | | | | | | QLA |
| | | | | | | TTK |
| 2202.1799 | 2201.9719 | −0.208 | −94 | 2864 | 2882 | MSELRVTLDPVQLES |
| | | | | | | SLLR |
| 2233.1135 | 2233.0076 | −0.1059 | −47 | 2462 | 2481 | EALAGLLVTYPNSQE |
| | | | | | | AEN |
| | | | | | | WK |
| 2233.1135 | 2233.2017 | 0.0882 | 39 | 2462 | 2481 | EALAGLLVTYPNSQE |
| | | | | | | AEN |
| | | | | | | WK |
| 2299.0217 | 2299.144 | 0.1223 | 53 | 3068 | 3088 | EMFSQLADLDDELDG |
| | | | | | | MG |
| | | | | | | AIGR |

Peptide Information

Tax_Id = 9606 Gene_Symbol = TSSK6
Conserved hypothetical protein

| | | | | Start | End | |
|---|---|---|---|---|---|---|
| Calc. Mass | Obsrv. Mass | ±da | ±ppm | Seq. | Seq. | Sequence |
| 856.4999 | 856.5223 | 0.0224 | 26 | 89 | 97 | AAQIAGAVR |
| 879.4907 | 879.4153 | −0.0754 | −86 | 55 | 62 | ATPAHRAR |
| 880.3876 | 880.4396 | 0.052 | 59 | 267 | 273 | GNMRSCR |
| 896.3825 | 896.4399 | 0.0574 | 64 | 267 | 273 | GNMRSCR |
| 912.4574 | 912.4597 | 0.0023 | 3 | 125 | 132 | LTDFGFGR |
| 1187.6136 | 1187.6656 | 0.052 | 44 | 271 | 279 | SCRVLLHMR |
| 1187.6136 | 1187.6656 | 0.052 | 44 | 271 | 279 | SCRVLLHMR |
| 1332.6768 | 1332.6146 | −0.0622 | −47 | 26 | 40 | GHQGGGPAASAPG |
| | | | | | | LR |
| 1413.771 | 1413.8057 | 0.0347 | 25 | 148 | 160 | GAPGHPLRPQEVR |
| 1487.7272 | 1487.7654 | 0.0382 | 26 | 111 | 122 | CENVLLSPDERR |
| 2299.2095 | 2299.144 | −0.0655 | −28 | 240 | 260 | LEAGWFQPFLQPRALGQ |
| | | | | | | GGAR |

Peptide Information

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3606 | 3613 | LMALGPIR |
| 870.5229 | 870.5385 | 0.0156 | 18 | 3606 | 3613 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 928.4669 | 928.4629 | −0.004 | −4 | 3522 | 3528 | KEVMEHR |
| 1021.5499 | 1021.5333 | −0.0166 | −16 | 2530 | 2537 | QQVQFMLK |
| 1021.5499 | 1021.5333 | −0.0166 | −16 | 2530 | 2537 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3291 | 3300 | VVKAQIQEQK |
| 1187.6201 | 1187.6656 | 0.0455 | 38 | 2736 | 2745 | NCPISAKLER |
| 1187.6201 | 1187.6656 | 0.0455 | 38 | 2736 | 2745 | NCPISAKLER |
| 1199.6896 | 1199.6674 | −0.0222 | −19 | 3628 | 3637 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4532 | 4542 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4517 | 4528 | QPVYDTTIRTGR |
| 1406.7386 | 1406.6833 | −0.0553 | −39 | 4517 | 4528 | QPVYDTTIRTGR |
| 1413.7809 | 1413.8057 | 0.0248 | 18 | 3135 | 3146 | ARQEQLELTLGR |
| 1420.7213 | 1420.6881 | −0.0332 | −23 | 2919 | 2930 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 4922 | 4933 | LNDALDRLEELK |
| 1465.7281 | 1465.7726 | 0.0445 | 30 | 4298 | 4309 | EETYNQLLDKGR |
| 1465.7316 | 1465.7726 | 0.041 | 28 | 4310 | 4323 | LMLLSRDDSGSGSK |
| 1487.7952 | 1487.7654 | −0.0298 | −20 | 3435 | 3447 | QTTGEEVLLIQEK |
| 1502.873 | 1502.8582 | −0.0148 | −10 | 380 | 391 | LLEVWIEFGRIK |
| 1532.6785 | 1532.7728 | 0.0943 | 62 | 3761 | 3773 | ELNPEEGEMVEEK |
| 1713.8728 | 1713.8539 | −0.0189 | −11 | 3102 | 3116 | HMLEEEGTLDLLG |
| | | | | | | LK |
| 1727.9149 | 1727.8947 | −0.0202 | −12 | 2151 | 2165 | KLLPQAEMFEHLS |
| | | | | | | GK |
| 1794.9636 | 1794.8103 | −0.1533 | −85 | 4976 | 4991 | QEFIDGILASKFPT |
| | | | | | | TK |
| 1838.8412 | 1838.927 | 0.0858 | 47 | 4830 | 4844 | ALIAEHQTFMEEM |
| | | | | | | TR |
| 2186.155 | 2185.9851 | −0.1699 | −78 | 1958 | 1978 | LLSDTVASDPGVLQE |
| | | | | | | QLA |
| | | | | | | TTK |
| 2202.1799 | 2201.9719 | −0.208 | −94 | 2843 | 2861 | MSELRVTLDPVQLES |
| | | | | | | SLLR |
| 2233.1135 | 2233.0076 | −0.1059 | −47 | 2441 | 2460 | EALAGLLVTYPNSQE |
| | | | | | | AEN |
| | | | | | | WK |
| 2233.1135 | 2233.2017 | 0.0882 | 39 | 2441 | 2460 | EALAGLLVTYPNSQE |
| | | | | | | AEN |
| | | | | | | WK |
| 2299.0217 | 2299.144 | 0.1223 | 53 | 3047 | 3067 | EMFSQLADLDDELDG |
| | | | | | | MG |
| | | | | | | AIGR |

Peptide Information

Tax_Id = 9606 Gene_Symbol = MACF1 Isoform
3 of Microtubule-actin cross-linking factor 1, isoforms 1/2/3/5

| | | | | Start | End | |
|---|---|---|---|---|---|---|
| Calc. Mass | Obsrv. Mass | ±da | ±ppm | Seq. | Seq. | Sequence |
| 870.5229 | 870.5385 | 0.0156 | 18 | 3680 | 3687 | LMALGPIR |
| 870.5229 | 870.5385 | 0.0156 | 18 | 3680 | 3687 | LMALGPIR |
| 879.4935 | 879.4153 | −0.0782 | −89 | 1839 | 1846 | FVTISGQK |
| 880.441 | 880.4396 | −0.0014 | −2 | 2205 | 2211 | DFTELQK |
| 910.4265 | 910.4448 | 0.0183 | 20 | 3420 | 3426 | YSEIQDR |
| 910.4265 | 910.4448 | 0.0183 | 20 | 3420 | 3426 | YSEIQDR |
| 928.4669 | 928.4629 | −0.004 | −4 | 3596 | 3602 | KEVMEHR |
| 1021.5499 | 1021.5333 | −0.0166 | −16 | 2495 | 2502 | QQVQFMLK |
| 1021.5499 | 1021.5333 | −0.0166 | −16 | 2495 | 2502 | QQVQFMLK |
| 1106.5034 | 1106.5583 | 0.0549 | 50 | 983 | 991 | LEEEVEACK |
| 1170.6841 | 1170.6443 | −0.0398 | −34 | 3256 | 3265 | VVKAQIQEQK |
| 1187.6201 | 1187.6656 | 0.0455 | 38 | 2701 | 2710 | NCPISAKLER |
| 1187.6201 | 1187.6656 | 0.0455 | 38 | 2701 | 2710 | NCPISAKLER |
| 1199.6896 | 1199.6674 | −0.0222 | −19 | 3702 | 3711 | AFSIDIIRHK |
| 1257.6797 | 1257.6525 | −0.0272 | −22 | 1471 | 1481 | QISEQLNALNK |
| 1257.6797 | 1257.6525 | −0.0272 | −22 | 1471 | 1481 | QISEQLNALNK |
| 1261.694 | 1261.6499 | −0.0441 | −35 | 345 | 354 | LLEVWIEFGR |
| 1287.6791 | 1287.6593 | −0.0198 | −15 | 4606 | 4616 | EKTLLPEDSQK |
| 1320.7271 | 1320.6016 | −0.1255 | −95 | 1835 | 1846 | GDLRFVTISGQK |
| 1406.7386 | 1406.6833 | −0.0553 | −39 | 4591 | 4602 | QPVYDTTIRTGR |
| 1406.7386 | 1406.6833 | −0.0553 | −39 | 4591 | 4602 | QPVYDTTIRTGR |
| 1413.7809 | 1413.8057 | 0.0248 | 18 | 3100 | 3111 | ARQEQLELTLGR |
| 1420.7213 | 1420.6881 | −0.0332 | −23 | 2884 | 2895 | TGSLEEMTQRLR |
| 1425.7156 | 1425.8075 | 0.0919 | 64 | 834 | 845 | NTISVKAVCDYR |
| 1428.7693 | 1428.7153 | −0.054 | −38 | 4996 | 5007 | LNDALDRLEELK |
| 1465.7281 | 1465.7726 | 0.0445 | 30 | 4372 | 4383 | EETYNQLLDK |
| | | | | | | GR |
| 1465.7316 | 1465.7726 | 0.041 | 28 | 4384 | 4397 | LMLLSRDDSGSGSK |
| 1487.7952 | 1487.7654 | −0.0298 | −20 | 3509 | 3521 | QTTGEEVLLIQEK |
| 1502.873 | 1502.8582 | −0.0148 | −10 | 345 | 356 | LLEVWIEFGRIK |
| 1532.6785 | 1532.7728 | 0.0943 | 62 | 3835 | 3847 | ELNPEEGEMVEEK |
| 1713.8728 | 1713.8539 | −0.0189 | −11 | 3067 | 3081 | HMLEEEGTLDLLGLK |
| 1727.9149 | 1727.8947 | −0.0202 | −12 | 2116 | 2130 | KLLPQAEMFEHLSGK |
| 1794.9636 | 1794.8103 | −0.1533 | −85 | 5050 | 5065 | QEFIDGILASKFPTTK |
| 1838.8412 | 1838.927 | 0.0858 | 47 | 4904 | 4918 | ALIAEHQTFMEEMTR |
| 2186.155 | 2185.9851 | −0.1699 | −78 | 1923 | 1943 | LLSDTVASDPGVLQE |
| | | | | | | QLA |
| | | | | | | TTK |
| 2202.1799 | 2201.9719 | −0.208 | −94 | 2808 | 2826 | MSELRVTLDPVQLES |
| | | | | | | SLLR |
| 2233.1135 | 2233.0076 | −0.1059 | −47 | 2406 | 2425 | EALAGLLVTYPNSQE |
| | | | | | | AEN |
| | | | | | | WK |
| 2233.1135 | 2233.2017 | 0.0882 | 39 | 2406 | 2425 | EALAGLLVTYPNSQE |
| | | | | | | AEN |
| | | | | | | WK |
| 2299.0217 | 2299.144 | 0.1223 | 53 | 3012 | 3032 | EMFSQLADLDDELDG |
| | | | | | | MG |
| | | | | | | AIGR |

Peptide Information

Tax_Id = 9606 Gene_Symbol = DNAH5 Dynein
heavy
chain 5, axonemal

| | | | | Start | End | |
|---|---|---|---|---|---|---|
| Calc. Mass | Obsrv. Mass | ±da | ±ppm | Seq. | Seq. | Sequence |
| 856.525 | 856.5223 | −0.0027 | −3 | 1408 | 1414 | QLNLLQK |
| 879.4683 | 879.4153 | −0.053 | −60 | 1654 | 1660 | RFSNIDK |
| 880.4774 | 880.4396 | −0.0378 | −43 | 1204 | 1211 | FALTAETK |
| 896.4407 | 896.4399 | −0.0008 | −1 | 747 | 753 | RNFSNMK |
| 910.488 | 910.4448 | −0.0432 | −47 | 1702 | 1709 | SLTGYLEK |
| 910.488 | 910.4448 | −0.0432 | −47 | 1702 | 1709 | SLTGYLEK |
| 912.4573 | 912.4597 | 0.0024 | 3 | 285 | 291 | AELEHWK |
| 928.5403 | 928.4629 | −0.0774 | −83 | 4440 | 4446 | IPAWWKK |
| 985.5941 | 985.582 | −0.0121 | −12 | 2503 | 2509 | RLELWLR |
| 985.5941 | 985.582 | −0.0121 | −12 | 2503 | 2509 | RLELWLR |
| 1005.5363 | 1005.6074 | 0.0711 | 71 | 820 | 827 | VNDLIEFR |
| 1021.4805 | 1021.5333 | 0.0528 | 52 | 2103 | 2111 | SVAMMVPDR |
| 1021.4805 | 1021.5333 | 0.0528 | 52 | 2103 | 2111 | SVAMMVPDR |
| 1106.5411 | 1106.5583 | 0.0172 | 16 | 326 | 333 | TWREMDIR |
| 1187.6816 | 1187.6656 | −0.016 | −13 | 4549 | 4558 | NMKLIESKPK |
| 1187.6816 | 1187.6656 | −0.016 | −13 | 4549 | 4558 | NMKLIESKPK |
| 1199.6995 | 1199.6674 | −0.0321 | −27 | 2585 | 2596 | AVLLIGEQGTAK |
| 1257.7566 | 1257.6525 | −0.1041 | −83 | 167 | 177 | LLSDIFIPALR |
| 1257.7566 | 1257.6525 | −0.1041 | −83 | 167 | 177 | LLSDIFIPALR |
| 1261.6212 | 1261.6499 | 0.0287 | 23 | 1299 | 1308 | VDTLHYAWEK |
| 1271.6553 | 1271.6659 | 0.0106 | 8 | 3711 | 3721 | TSIIDFTVTMK |
| 1332.7369 | 1332.6146 | −0.1223 | −92 | 3210 | 3222 | LKEASESVAALSK |
| 1413.8577 | 1413.8057 | −0.052 | −37 | 166 | 177 | RLLSDIFIPALR |
| 1428.7482 | 1428.7153 | −0.0329 | −23 | 3698 | 3710 | LPNPAYTPEISAR |
| 1502.9153 | 1502.8582 | −0.0571 | −38 | 1119 | 1132 | LVSVLSTIINSTKK |
| 1794.7972 | 1794.8103 | 0.0131 | 7 | 748 | 761 | NFSNMKMMLAEYQR |
| 1838.8668 | 1838.927 | 0.0602 | 33 | 3501 | 3515 | ERWTEQSQEFAAQTK |
| 2266.176 | 2266.0767 | −0.0993 | −44 | 957 | 975 | ELLSHFNHQNMDALL |
| | | | | | | KV |
| | | | | | | TR |

Peptide Information

| | | | | Start | End | |
|---|---|---|---|---|---|---|
| Calc. Mass | Obsrv. Mass | ±da | ±ppm | Seq. | Seq. | Sequence |
| 924.4897 | 924.4626 | −0.0271 | −29 | 385 | 392 | EILNNHGK |
| 985.5425 | 985.582 | 0.0395 | 40 | 157 | 166 | DAAPGASKLR |
| 985.5425 | 985.582 | 0.0395 | 40 | 157 | 166 | DAAPGASKLR |
| 1021.6153 | 1021.5333 | −0.082 | −80 | 188 | 196 | GVVDHLLLR |
| 1021.6153 | 1021.5333 | −0.082 | −80 | 188 | 196 | GVVDHLLLR |
| 1254.6161 | 1254.6615 | 0.0454 | 36 | 2 | 11 | QPWHGKAMQR |
| 1257.5422 | 1257.6525 | 0.1103 | 88 | 496 | 505 | NNEFPVFDEF |
| 1257.5422 | 1257.6525 | 0.1103 | 88 | 496 | 505 | NNEFPVFDEF |
| 1271.7206 | 1271.6659 | −0.0547 | −43 | 303 | 315 | GGSPAVTLLISEK |
| 1287.6652 | 1287.6593 | −0.0059 | −5 | 12 | 25 | ASEAGATAPKASAR |
| 1413.6719 | 1413.8057 | 0.1338 | 95 | 220 | 231 | ISAPNEFDVMFK |
| 1479.7472 | 1479.7794 | 0.0322 | 22 | 174 | 187 | LSRDDISTAAGMVK |

Peptide Information

Tax_Id = 9606 Gene_Symbol = DLG5 Isoform 4
of Disks
large homolog 5

| | | | | Start | End | |
|---|---|---|---|---|---|---|
| Calc. Mass | Obsrv. Mass | ±da | ±ppm | Seq. | Seq. | Sequence |
| 879.3876 | 879.4153 | 0.0277 | 31 | 132 | 138 | DDVDMLR |
| 880.4523 | 880.4396 | −0.0127 | −14 | 206 | 212 | DYDALRK |
| 924.4421 | 924.4626 | 0.0205 | 22 | 1766 | 1772 | LEQEYSR |
| 985.5537 | 985.582 | 0.0283 | 29 | 139 | 146 | RENGQLLR |
| 985.5537 | 985.582 | 0.0283 | 29 | 139 | 146 | RENGQLLR |
| 1187.5917 | 1187.6656 | 0.0739 | 62 | 712 | 722 | AHGPEVQAHNK |
| 1187.5917 | 1187.6656 | 0.0739 | 62 | 712 | 722 | AHGPEVQAHNK |
| 1261.6205 | 1261.6499 | 0.0294 | 23 | 358 | 368 | KAANEEMEALR |
| 1406.7526 | 1406.6833 | −0.0693 | −49 | 1495 | 1506 | LADVEQELSFKK |
| 1406.7526 | 1406.6833 | −0.0693 | −49 | 1495 | 1506 | LADVEQELSFKK |
| 1420.7026 | 1420.6881 | −0.0145 | −10 | 1562 | 1575 | DDNSATKTLSAAAR |
| 1487.8315 | 1487.7654 | −0.0661 | −44 | 339 | 351 | LQTEVELAESKLK |
| 1502.7632 | 1502.8582 | 0.095 | 63 | 359 | 371 | AANEEMEALRQIK |
| 1727.9539 | 1727.8947 | −0.0592 | −34 | 1243 | 1259 | VQKGSEPLGISIVSG |
| | | | | | | EK |

Peptide Information

Tax_Id = 9606 Gene_Symbol = LMCD1
Uncharacterized protein

| | | | | Start | End | |
|---|---|---|---|---|---|---|
| Calc. Mass | Obsrv. Mass | ±da | ±ppm | Seq. | Seq. | Sequence |
| 870.5043 | 870.5385 | 0.0342 | 39 | 7 | 14 | DLNPGVKK |
| 870.5043 | 870.5385 | 0.0342 | 39 | 7 | 14 | DLNPGVKK |
| 1106.5623 | 1106.5583 | −0.004 | −4 | 15 | 24 | MSLGQLQSAR |
| 1420.6063 | 1420.6881 | 0.0818 | 58 | 33 | 44 | GTCSGFEPHSWR |
| 2265.9951 | 2266.0767 | 0.0816 | 36 | 25 | 44 | GVACLGCKGTCSGFEPH |

19 growth-inhibiting protein 25

Identification of a human cell growth inhibiting gene

20 fibrinogen gamma

Fibrinogen (factor I) is a soluble plasma glycoprotein, synthesised by the liver, that is converted by thrombin into fibrin during blood coagulation. It consists of alpha, beta and gamma chain. This is achieved through processes in the coagulation cascade that activate the zymogen prothrombin to the serine protease thrombin, which is responsible for converting fibrinogen into fibrin. Fibrin is then cross linked by factor XIII to form a clot. FXIIIa stabilizes fibrin further by incorporation of the fibrinolysis inhibitors alpha-2-antiplasmin and TAFI (thrombin activatable fibrinolysis inhibitor, procarboxypeptidase B), and binding to several adhesive proteins of various cells. Both the activation of Factor XIII by thrombin and plasminogen activator (t-PA) are catalyzed by fibrin. Fibrin specifically binds the activated coagulation factors factor Xa and thrombin and entraps them in the network of fibers, thus functioning as a temporary inhibitor of these enzymes, which stay active and can be released during fibrinolysis. Recent research has shown that fibrin plays a key role in the inflammatory response and development of rheumatoid arthritis.

21 Chain L, Crystal Structure Of Human Fibrinogen

Please refer to above

22 growth-inhibiting protein 25

Refer to Nr 19

23 Chain A of IgM

Immunoglobulin M, or IgM for short, is a basic antibody that is produced by B cells. IgM is by far the physically largest antibody in the human circulatory system. It is the first antibody to appear in response to initial exposure to antigen. IgM forms polymers where multiple immunoglobulins are covalently linked together with disulfide bonds, mostly as a pentamer but also as a hexamer. IgM has a molecular mass of approximately 900 kDa (in its pentamer form). Because each monomer has two antigen binding sites, a pentameric IgM has 10 binding sites. Typically, however, IgM cannot bind 10 antigens at the same time because the large size of most antigens hinders binding to nearby sites. IgM antibodies appear early in the course of an infection and usually reappear, to a lesser extent, after further exposure. IgM antibodies do not pass across the human placenta (only isotype IgG). These two biological properties of IgM make it useful in the diagnosis of infectious diseases. Demonstrating IgM antibodies in a patient's serum indicates recent infection, or in a neonate's serum indicates intrauterine infection

24 Chain A, Crystal Structure Of The Fab Fragment Of A Human Monoclonal Igm Cold Agglutinin

Cold agglutinin disease is an autoimmune disease characterized by the presence of high concentrations of circulating antibodies, usually IgM, directed against red blood cells. It is a form of autoimmune hemolytic anemia, specifically one in which antibodies only bind red blood cells at low body temperatures, typically 28-31° C.

25 immunoglobulin light chain

Immunoglobulin is a large Y-shaped protein produced by B-cells that is used by the immune system to identify and neutralize foreign objects such as bacteria and viruses. Immunoglobin consists of light chain and heavy chain. The antibody recognizes a unique part of the foreign target, termed an antigen. Each tip of the “Y” of an antibody contains a paratope (a structure analogous to a lock) that is specific for one particular epitope (similarly analogous to a key) on an antigen, allowing these two structures to bind together with precision. Using this binding mechanism, an antibody can tag a microbe or an infected cell for attack by other parts of the immune system, or can neutralize its target directly (for example, by blocking a part of a microbe that is essential for its invasion and survival). The production of antibodies is the main function of the humoral immune system.

26 Chain C, Molecular Basis For Complement Recognition

The complement system helps or “complements” the ability of antibodies and phagocytic cells to clear pathogens from an organism. It is part of the immune system called the innate immune system that is not adaptable and does not change over the course of an individual's lifetime. However, it can be recruited and brought into action by the adaptive immune system.

The complement system consists of a number of small proteins found in the blood, generally synthesized by the liver, and normally circulating as inactive precursors (pro-proteins). When stimulated by one of several triggers, proteases in the system cleave specific proteins to release cytokines and initiate an amplifying cascade of further cleavages. The end-result of this activation cascade is massive amplification of the response and activation of the cell-killing membrane attack complex. Over 25 proteins and protein fragments make up the complement system, including serum proteins, serosal proteins, and cell membrane receptors. They account for about 5% of the globulin fraction of blood serum.

27 immunoglobulin light chain

FIG. 220

Description

PROCSS OF AFCC01 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until its concentration is 5%.

3,to cool down the suspension to 2-4 C.

4, to go to centrifugation at temperature of 2-4 C, obtain the paste, called paste31.

5, to dissolve above paste with buffer (PH8.50), dilution ratio is 1:9.

6, to go to centrifugation, obtain the supernatant

7, to perform filtration with depth filters such as 10 cp, 90 sp followed by 0.45 μm, obtain the clear filtrate.

8, to concentrate the solution to 5%? With 10 k ultra-filtration membrane, then dialysis with 10 volume of cold WFI,

9, to carry out DV20 filtration

10,to adjust the PH value to 7.00.

11, to add albumin to concentration of 2.5%? as stabilizer.

12, to go to sterile filtration and filling.

FIG. 221

Description

PROCSS OF AFCC02 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the paste, called paste31.

5, to dissolve above paste with buffer (PH8.50), dilution ratio is 1:9.

6, to go to centrifugation, obtain the supernatant

7, to perform filtration with depth filters such as 10 cp, 90 sp followed by 0.45 μm, obtain the clear filtrate.

8, to concentrate the solution to 5%? With 10 k ultra-filtration membrane, collect the permeate.

9, to concentrate the permeate to 3%? With 1-3 k ultra-filtration membrane, then dialysis with 10 volume of cold WFI

10, to carry out DV20 filtration

11,to adjust the PH value to 7.00.

12, to add albumin to concentration of 2.5%? as stabilizer.

13, to go to sterile filtration and filling.

FIG. 222

description

PROCSS OF AFCC03 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until its concentration is 5%.

3,to cool down the suspension to 2-4 C.

4, to go to centrifugation at temperature of 2-4 C, obtain the paste, called paste31.

5, to dissolve above paste with buffer (PH8.50), dilution ratio is 1:9.

6, to go to centrifugation, collect the paste

7, to dissolve above paste with buffer (PH8.50?), dilution ratio is 1:9?

7, to perform filtration with depth filters such as 10 cp, 90 sp followed by 0.45 μm, obtain the clear filtrate.

8, to concentrate the solution to 5%? With 10 k ultra-filtration membrane, then dialysis with 10 volume of cold WFI,

9, to carry out DV20 filtration

10,to adjust the PH value to 7.00.

11, to add albumin to concentration of 2.5%? as stabilizer.

12, to go to sterile filtration and filling.

FIG. 223

Description

PROCSS OF AFCC04 FROM FrIII PASTE

1, Firstly to dissolve the Fr.III paste with WFI, dilution ratio is 1:4,then add sodium chloride to concentration of 150 mM and adjust PH value of the suspension to about 7.00, keep temperature of the suspension to 23-25 C, to agitate at sufficient rate until fully dissolved.

2, to add PEG to the suspension until its concentration is 5%.

3,to cool down the suspension to 2-4 C.

4, to go to centrifugation at temperature of 2-4 C, obtain the paste, called paste31.

5, to dissolve above paste with buffer (PH8.50), dilution ratio is 1:9.

6, to go to centrifugation, collect the paste

7, to dissolve above paste with buffer (PH8.50?), dilution ratio is 1:9?

8, to perform filtration with depth filters such as 10 cp, 90 sp followed by 0.45 μm, obtain the clear filtrate.

9, to concentrate the solution to 5%? With 10 k ultra-filtration membrane, collect permeate.

10, to concentrate the solution to 3%? With 1-3 k ultra-filtration membrane, then dialysis with 10 volume of cold WFI,

11, to carry out DV20 filtration

12,to adjust the PH value to 7.00.

13, to add albumin to concentration of 2.5%? as stabilizer.

14, to go to sterile filtration and filling.

PROCESS OF AFCC05 FROM FrIII PASTE

FIG. 224

Description

1, Firstly to dissolve the Fr.III paste with WFI, dilution ratio is 1:4,then add sodium chloride to concentration of 150 mM and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 nm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect the paste, called paste32.

12, to dissolve the paste 32 with WFI, contain 150 mmol sodium chloride, dilution ratio is 1:100

13, to concentrate the solution to 5%? With 10 k ultra-filtration membrane, collect the permeate.

14, to concentrate the permeate to 3%? With 1-3 k ultra-filtration membrane, then dialysis with 10 volume of cold WFI

15, to carry out DV20 filtration

16,to adjust the PH value to 7.00.

17, to add albumin to concentration of 2.5%? as stabilizer.

18, to go to sterile filtration and filling.

FIG. 225—Flow chart of AFCC 06 PROCSS FROM FrIII PASTE

Description

PROCSS OF AFCC06 FROM FrIII PASTE

1, Firstly to dissolve the Fr.III paste with WFI, dilution ratio is 1:4,then add sodium chloride to concentration of 150 mM and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect the paste, called paste32.

12, to dissolve the paste 32 with WFI, contain 150 mmol sodium chloride, dilution ratio is 1:100

13, to concentrate the solution to 5%? With 10 k ultra-filtration membrane, then dialysis with 10 volume of cold WFI

14, to carry out DV20 filtration

15,to adjust the PH value to 7.00.

16, to add albumin to concentration of 2.5%? as stabilizer.

17, to go to sterile filtration and filling.

FIG. 226—Flow chart of AFCC 07 PROCSS FROM FrIII PASTE

Description

PROCSS OF AFCC07 FROM FrIII PASTE

1, Firstly to dissolve the Fr.III paste with WFI, dilution ratio is 1:4,then add sodium chloride to concentration of 150 mM and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the paste, called 33.

14, to dissolve the paste 33 with WFI, contain 150 mmol sodium chloride, dilution ratio is 1:100

15, to concentrate the solution to 5%? With 10 k ultra-filtration membrane, then dialysis with 10 volume of cold WFI

16, to carry out DV20 filtration

17,to adjust the PH value to 7.00.

18, to add albumin to concentration of 2.5%? as stabilizer.

19, to go to sterile filtration and filling.

FIG. 227—Flow chart of AFCC 08 PROCSS FROM FrIII PASTE

Description

PROCSS OF AFCC08 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the paste, called 33.

14, to dissolve the paste 33 with WFI, contain 150 mmol sodium chloride, dilution ratio is 1:100

15, to concentrate the solution to 5%? With 10 k ultra-filtration membrane, collect permeate

16, to concentrate the solution to 3%? With 1-3 k ultra-filtration membrane, then dialysis with 10 volume of cold WFI

17, to carry out DV20 filtration

18,to adjust the PH value to 7.00.

19, to add albumin to concentration of 2.5%? as stabilizer.

20, to go to sterile filtration and filling.

FIG. 228—Flow chart of AFCC 09 PROCSS FROM FrIII PASTE

Description

PROCSS OF AFCC09 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the supernatant.

14,to perform filtration with depth filters such as 10 cp, 90 sp followed by 0.45 μm, obtain the clear filtrate.

15, to load filtrate to column (DEAE FF),collect elute.

16, to concentrate the solution to 5%? With 10 k ultra-filtration membrane, then dialysis with 10 volume of cold WFI

17, to carry out DV20 filtration

18,to adjust the PH value to 7.00.

19, to add albumin to concentration of 2.5%? as stabilizer.

20, to go to sterile filtration and filling.

FIG. 229—Flow chart of AFCC 10 PROCSS FROM FrIII PASTE

Description

PROCSS OF AFCC10 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the supernatant.

14,to perform filtration with depth filters such as 10 cp, 90 sp followed by 0.45 μm, obtain the clear filtrate.

15, to load to column (DEAE FF),collect elute.

16, to concentrate the solution to 5%? With 10 k ultra-filtration membrane, collect permeate.

17, to concentrate the permeate to 3%? With 1-3 k ultra-filtration membrane, then dialysis with 10 volume of cold WFI

18, to carry out DV20 filtration

19,to adjust the PH value to 7.00.

20, to add albumin to concentration of 2.5%? as stabilizer.

21, to go to sterile filtration and filling.

FIG. 230—Flow chart of AFCC 11 PROCSS FROM FrIII PASTE

Description

PROCSS OF AFCC11 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the supernatant.

14,to perform filtration with depth filters such as 10 cp, 90 sp followed by 0.45 μm, obtain the clear filtrate.

15, to load to column (DEAE FF),collect flowthrough

16, to add alcohol to flowthrough until its concentration is 20%,adjust PH value to 5.80

17, to go to centrifugation at temperature of −4-6 C, obtain the paste.

18,to dissolve the paste with WFI, dilution ratio is 1:20?.

19,to perform filtration with depth filters such as 10 cp, 90 sp followed by 0.45 μm, obtain the clear filtrate

20, to concentrate the solution to 5%? With 10 k ultra-filtration membrane, then dialysis with 10 volume of cold WFI

21, to carry out DV20 filtration

22,to adjust the PH value to 7.00.

23, to add albumin to concentration of 2.5%? as stabilizer.

24, to go to sterile filtration and filling.

FIGS. 231A&B—Flow chart of AFCC 12 PROCSS FROM FrIII PASTE

Description

PROCSS OF AFCC12 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, remove the A-50 resin from the solution. collect the supernatant.

10,to add alcohol to supernatant until its concentration is 8%,adjust PH value to 7.00

11, to go to centrifugation at temperature of −1-1 C, collect supernatant

12, to add alcohol to supernatant until its concentration is 20%,adjust PH value to 5.80

13, to go to centrifugation at temperature of −4-6 C, obtain the supernatant.

14,to perform filtration with depth filters such as 10 cp, 90 sp followed by 0.45 μm, obtain the clear filtrate.

15, to load to column (DEAE FF),collect flowthrough

16, to add alcohol to flowthrough until its concentration is 20%,adjust PH value to 5.80

17, to go to centrifugation at temperature of −4-6 C, obtain the paste.

18,to dissolve the paste with WFI, dilution ratio is 1:20?.

19,to perform filtration with depth filters such as 10 cp, 90 sp followed by 0.45 μm, obtain the clear filtrate

20, to concentrate the solution to 5%? With 10 k ultra-filtration membrane, collect permeate.

21, to concentrate the permeate to 3%? With 1-3 k ultra-filtration membrane, then dialysis with 10 volume of cold WFI

22, to carry out DV20 filtration

23,to adjust the PH value to 7.00.

24, to add albumin to concentration of 2.5%? as stabilizer.

25, to go to sterile filtration and filling.

FIG. 232—Flow chart of AFCC 13 PROCSS FROM FrIII PASTE

Description

PROCSS OF AFCC13 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, collect the A-50 resin from the solution.

10,to wash the A-50 resin, collect washing solution

11,to adjust the PH value of the solution to ?

12,to go to centrifugation at temperature of −1-1 C?, collect paste

13,to dissolve the paste with WFI, dilution ratio is 1:100?.

14,to perform filtration with depth filters such as 10 cp, 90 sp followed by 0.45 μm, obtain the clear filtrate

15, to concentrate the solution to 2.5%? With 10 k ultra-filtration membrane, then dialysis with 10 volume of cold WFI

16, to carry out DV20 filtration

17,to adjust the PH value to 7.00.

18, to add albumin to concentration of 2.5%? as stabilizer.

19, to go to sterile filtration and filling.

Description

FIG. 233—Flow chart of AFCC 14 PROCSS FROM FrIII PASTE

PROCSS OF AFCC14 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, collect the A-50 resin from the solution.

10,to wash the A-50 resin, collect washing solution

11,to adjust the PH value of the solution to ?

12,to go to centrifugation at temperature of −1-1 C?, collect paste

13,to dissolve the paste with WFI, dilution ratio is 1:100?.

14,to perform filtration with depth filters such as 10 cp, 90 sp followed by 0.45 μm, obtain the clear filtrate

15, to concentrate the solution to 2.5%? With 10 k ultra-filtration membrane, collect permeate.

16,to concentrate the permeate to 2.5%? With 1-3 k ultra-filtration membrane, then dialysis with 10 volume of cold WFI

17, to carry out DV20 filtration

18,to adjust the PH value to 7.00.

19, to add albumin to concentration of 2.5%? as stabilizer.

20, to go to sterile filtration and filling.

Description

FIG. 234—Flow chart of AFCC 15 PROCSS FROM FrIII PASTE

PROCSS OF AFCC15 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 for 6 hours.

7 to cool down the solution to temperature below 10 C and adjust PH value to about ?

8, to add A-50 resin to the solution for PCC adsorption

9, collect the A-50 resin from the solution.

10,to wash the A-50 resin, collect washing solution

11,to adjust the PH value of the solution to ?

12,to go to centrifugation at temperature of −1-1?, collect supernatant.

13,to perform filtration with depth filters such as 10 cp, 90 sp followed by 0.45 μm, obtain the clear filtrate

14, to concentrate the solution to 2.5%? With 10 k ultra-filtration membrane, then dialysis with 10 volume of cold WFI

15, to carry out DV20 filtration

16,to adjust the PH value to 7.00.

17, to add albumin to concentration of 2.5%? as stabilizer.

18, to go to sterile filtration and filling.

Description

FIG. 235—Flow chart of AFCC 16 PROCSS FROM FrIII PASTE

PROCSS OF AFCC16 FROM FrIII PASTE

1, Firstly to dissolve the Fr.III paste with WFI, dilution ratio is 1:4,then add sodium chloride to concentration of 150 mM

and adjust PH value of the suspension to about 7.00, keep temperature of the suspension to 23-25 C, to agitate at sufficient rate until fully dissolved.

2, to add PEG to the suspension until concentration is 5%.

3,to cool down the suspension to 2-4 C.

4, to go to centrifugation at temperature of 2-4 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7 to cool down the solution to temperature below 10 C and adjust PH value to about ?

8, to add A-50 resin to the solution for PCC adsorption

9, collect the A-50 resin from the solution.

10,to wash the A-50 resin, collect washing solution

11,to adjust the PH value of the solution to ?

12,to go to centrifugation at temperature of −1-1 C?, collect supernatant.

13,to perform filtration with depth filters such as 10 cp, 90 sp followed by 0.45 μm, obtain the clear filtrate

14, to concentrate the solution to 2.5%? With 10 k ultra-filtration membrane, collect permeate.

15,to concentrate the permeate to 2.5%? With 1-3 k ultra-filtration membrane, then dialysis with 10 volume of cold WFI

16, to carry out DV20 filtration

17,to adjust the PH value to 7.00.

18, to add albumin to concentration of 2.5%? as stabilizer.

19, to go to sterile filtration and filling.

AFOD KH sequence result

FIG. 236—AFOD KH & Fr. IV

| | |
|---|---|
| 1 | CP 98 kDa protein |
| 2 | CP Ceruloplasmin |
| 3 | KRT2 Keratin, type II cytoskeletal 2 epidermal |
| 4 | no matched protein found |
| 5 | no matched protein found |
| 6 | no matched protein found |
| 7 | no matched protein found |
| 8 | APOA1 Apolipoprotein A-1 |
| 9 | APOA1 Apolipoprotein A-1 |
| 10 | APOA1 Apolipoprotein A-1 |
| 11 | APOA1 Apolipoprotein A-1 |
| 12 | Human albumin |
| 13 | Transferrin |
| 14 | Vimentin |
| 15 | Haptoqlobin |

AFOD KH

FIG. 237—AFOD KH

1 CP 98 kDa protein

Nup98 and Nup96 play a role in the bidirectional transport across the nucleoporin complex (NPC). The repeat domain in

Nup98 has a direct role in the transport.

Signal-mediated nuclear import and export proceed through the nuclear pore complex (NPC), which is composed of approximately 50 unique proteins collectively known as nucleoporins. The 98 kD nucleoporin is generated through a biogenesis pathway that involves synthesis and proteolytic cleavage of a 186 kD precursor protein. This cleavage results in the 98 kD nucleoporin as well as a 96 kD nucleoporin, both of which are localized to the nucleoplasmic side of the NPC. Rat studies show that the 98 kD nucleoporin functions as one of several docking site nucleoporins of transport substrates. The human gene has been shown to fuse to several genes following chromosome translocatons in acute myelogenous leukemia (AML) and T-cell acute lymphocytic leukemia (T-ALL). This gene is one of several genes located in the imprinted gene domain of 11p15.5, an important tumor-suppressor gene region. Alterations in this region have been associated with the Beckwith-Wiedemann syndrome, Wilms tumor, rhabdomyosarcoma, adrenocortical carcinoma, and lung, ovarian, and breast cancer.

2 CP Ceruloplasmin

Ceruloplasmin (or caeruloplasmin) is a ferroxidase enzyme that in humans is encoded by the CP gene. Ceruloplasmin is the major copper-carrying protein in the blood, and in addition plays a role in iron metabolism. Another protein, hephaestin, is noted for its homology to ceruloplasmin, and also participates in iron and probably copper metabolism. Ceruloplasmin carries about 70% of the total copper in human plasma while albumin carries about 15%. The rest is accounted for by macroglobulins. Albumin may be confused at times to have a greater importance as a copper carrier because it binds copper less tightly than ceruloplasmin. Ceruloplasmin exhibits a copper-dependent oxidase activity, which is associated with possible oxidation of Fe2+ (ferrous iron) into Fe3+ (ferric iron), therefore assisting in its transport in the plasma in association with transferrin, which can carry iron only in the ferric state. The molecular weight of human ceruloplasmin is reported to be 151 kDa.

3 KRT2 Keratin, type II cytoskeletal 2 epidermal

Keratin, type II cytoskeletal 2 epidermal is a protein that in humans is encoded by the KRT86 gene. The protein encoded by this gene is a member of the keratin gene family. As a type II hair keratin, it is a basic protein which heterodimerizes with type I keratins to form hair and nails. The type II hair keratins are clustered in a region of chromosome 12q13 and are grouped into two distinct subfamilies based on structure similarity. One subfamily, consisting of KRTHB1, KRTHB3, and KRTHB6, is highly related. The other less-related subfamily includes KRTHB2, KRTHB4, and KRTHB5. All hair keratins are expressed in the hair follicle; this hair keratin, as well as KRTHB1 and KRTHB3, is found primarily in the hair cortex. Mutations in this gene and KRTHB1 have been observed in patients with a rare dominant hair disease, monilethrix. 4 KH3 Protein—No matched protein found, now named KH3 Protein

| | | | | | |
|---|---|---|---|---|---|
| 891.4166891.451 | 0.0344 | 39 | 78 | 84 | ESEDQKR |
| 982.4734982.4398 | −0.0336 | −34 | 1 | 9 | MGGTTSTRR |

| | |
|---|---|
| 155/G6 | Instr./Gel Origin |
| [1] Sample Project | |
| 20111201 | |
| Accession No. | Protein Name |
| IPI00893693 | Tax_Id=9606 Gene_Symbol=CCDC88A |
| 137 kDa protein | |
| Instrument Sample Name | |

### Peptide Information

| | | | | Start | End | |
|---|---|---|---|---|---|---|
| Calc. Mass | Obsrv. Mass | ±da | ±ppm | Seq. | Seq. | Sequence |
| 879.4968 | 879.4153 | -0.0815 | -93 | 635 | 642 | KSSMVALK |
| 880.5138 | 880.4396 | -0.0742 | -84 | 129 | 135 | YKLLESK |
| 896.4584 | 896.4399 | -0.0185 | -21 | 365 | 371 | NLEVEHR |
| 985.5789 | 985.582 | 0.0031 | 3 | 16 | 23 | LRQQAEIK |
| 985.5789 | 985.582 | 0.0031 | 3 | 16 | 23 | LRQQAEIK |
| 1021.5272 | 1021.5333 | 0.0061 | 6 | 961 | 969 | ESSLSRQSK |
| 1021.5425 | 1021.5333 | -0.0092 | -9 | 178 | 185 | NYEALKQR |
| 1187.6267 | 1187.6656 | 0.0389 | 33 | 383 | 392 | QKGQLEDLEK |
| | 1187.6656 | | | | | |
| 1187.6267 | 1187.6656 | 0.0389 | 33 | 383 | 392 | QKGQLEDLEK |
| 1199.5903 | 1199.6674 | 0.0771 | 64 | 435 | 444 | ETEVLQTDHK |
| 1254.6212 | 1254.6615 | 0.0403 | 32 | 345 | 355 | QASEYESLISK |
| 1406.7274 | 1406.6833 | -0.0441 | -31 | 372 | 382 | DLEDRYNQLLK |
| 1406.7274 | 1406.6833 | -0.0441 | -31 | 372 | 382 | DLEDRYNQLLK |
| 1428.6754 | 1428.7153 | 0.0399 | 28 | 909 | 921 | SVSGKTPGDFYDR |
| 1479.6996 | 1479.7794 | 0.0798 | 54 | 875 | 887 | SSSQENLLDEVMK |
| 1502.8425 | 1502.8582 | 0.0157 | 10 | 78 | 90 | TLVTLREDLVSEK |
| 1727.9286 | 1727.8947 | -0.0339 | -20 | 223 | 237 | LIEVERNNATLQAEK |
| 2213.1084 | 2213.2441 | 0.1357 | 61 | 935 | 955 | KTEDTYFISSAGKPTPG |
| 2233.0918 | 2233.0076 | -0.0842 | -38 | 515 | 532 | T QGK |
| | | | | | | TLLEQNMESKDLFHVE |
| | | | | | | Q |
| | | | | | | R |
| 2233.0918 | 2233.2017 | 0.1099 | 49 | 515 | 532 | TLLEQNMESKDLFHVE |
| | | | | | | Q |
| | | | | | | R |

IPI01012199 Tax_Id=9606 Gene_Symbol=MACF1
Uncharacterized protein
Protein Group
IPI00256861 Tax_Id=9606 Gene_Symbol=MACF1
Isoform 2 of
Microtubule-actin cross-linking factor 1, isoforms 1/2/3/5

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5385 | 0.0156 | 18 | 3736 | 3743 | LMALGPIR |
| 870.5229 | 870.5385 | 0.0156 | 18 | 3736 | 3743 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 910.4265 | 910.4448 | 0.0183 | 20 | 3476 | 3482 | YSEIQDR |
| 910.4265 | 910.4448 | 0.0183 | 20 | 3476 | 3482 | YSEIQDR |
| 928.4669 | 928.4629 | −0.004 | −4 | 3652 | 3658 | KEVMEHR |
| 1021.5499 | 1021.5333 | −0.0166 | −16 | 2551 | 2558 | QQVQFMLK |
| 1021.5499 | 1021.5333 | −0.0166 | −16 | 2551 | 2558 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3312 | 3321 | VVKAQIQEQK |
| 1187.6201 | 1187.6656 | 0.0455 | 38 | 2757 | 2766 | NCPISAKLER |
| 1187.6201 | 1187.6656 | 0.0455 | 38 | 2757 | 2766 | NCPISAKLER |
| 1199.6896 | 1199.6674 | −0.0222 | −19 | 3758 | 3767 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4662 | 4672 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4647 | 4658 | QPVYDTTIRTGR |
| 1406.7386 | 1406.6833 | −0.0553 | −39 | 4647 | 4658 | QPVYDTTIRTGR |
| 1413.7809 | 1413.8057 | 0.0248 | 18 | 3156 | 3167 | ARQEQLELTLGR |
| 1420.7213 | 1420.6881 | −0.0332 | −23 | 2940 | 2951 | TGSLEEMTQRLR |
| 1425.7156 | 1425.8075 | 0.0919 | 64 | 869 | 880 | NTISVKAVCDYR |
| 1428.7693 | 1428.7153 | −0.054 | −38 | 5052 | 5063 | LNDALDRLEELK |
| 1465.7281 | 1465.7726 | 0.0445 | 30 | 4428 | 4439 | EETYNQLLDKGR |
| 1465.7316 | 1465.7726 | 0.041 | 28 | 4440 | 4453 | LMLLSRDDSGSGSK |
| 1487.7952 | 1487.7654 | −0.0298 | −20 | 3565 | 3577 | QTTGEEVLLIQEK |
| 1502.873 | 1502.8582 | −0.0148 | −10 | 380 | 391 | LLEVWIEFGRIK |
| 1532.6785 | 1532.7728 | 0.0943 | 62 | 3891 | 3903 | ELNPEEGEMVEEK |
| 1713.8728 | 1713.8539 | −0.0189 | −11 | 3123 | 3137 | HMLEEEGTLDLLGLK |
| 1727.9149 | 1727.8947 | −0.0202 | −12 | 2151 | 2165 | KLLPQAEMFEHLSGK |
| 1794.9636 | 1794.8103 | −0.1533 | −85 | 5106 | 5121 | QEFIDGILASKFPTTK |
| 1838.8412 | 1838.927 | 0.0858 | 47 | 4960 | 4974 | ALIAEHQTFMEEMTR |
| 2186.155 | 2185.9851 | −0.1699 | −78 | 1958 | 1978 | LLSDTVASDPGVLQE |
| | | | | | | QLA |
| | | | | | | TTK |
| 2202.1799 | 2201.9719 | −0.208 | −94 | 2864 | 2882 | MSELRVTLDPVQLESS |
| | | | | | | LLR |
| 2233.1135 | 2233.0076 | −0.1059 | −47 | 2462 | 2481 | EALAGLLVTYPNSQE |
| | | | | | | AEN |
| | | | | | | WK |
| 2233.1135 | 2233.2017 | 0.0882 | 39 | 2462 | 2481 | EALAGLLVTYPNSQE |
| | | | | | | AEN |
| | | | | | | WK |
| 2299.0217 | 2299.144 | 0.1223 | 53 | 3068 | 3088 | EMFSQLADLDDELDG |
| | | | | | | MG |
| | | | | | | AIGR |

Peptide Information

| | Obsrv. | | | Start | End | |
|---|---|---|---|---|---|---|
| Calc. Mass | Mass | ±da | ±ppm | Seq. | Seq. | Sequence |
| 856.4999 | 856.5223 | 0.0224 | 26 | 89 | 97 | AAQIAGAVR |
| 879.4907 | 879.4153 | −0.0754 | −86 | 55 | 62 | ATPAHRAR |
| 880.3876 | 880.4396 | 0.052 | 59 | 267 | 273 | GNMRSCR |
| 896.3825 | 896.4399 | 0.0574 | 64 | 267 | 273 | GNMRSCR |
| 912.4574 | 912.4597 | 0.0023 | 3 | 125 | 132 | LTDFGFGR |
| 1187.6136 | 1187.6656 | 0.052 | 44 | 271 | 279 | SCRVLLHMR |
| 1187.6136 | 1187.6656 | 0.052 | 44 | 271 | 279 | SCRVLLHMR |
| 1332.6768 | 1332.614647 | −0.0622 | — | 26 | 40 | GHQGGGPAASAPGLR |
| 1413.771 | 1413.8057 | 0.0347 | 25 | 148 | 160 | GAPGHPLRPQEVR |
| 1487.7272 | 1487.7654 | 0.0382 | 26 | 111 | 122 | CENVLLSPDERR |
| 2299.2095 | 2299.14428 | −0.0655 | — | 240 | 260 | LEAGWFQPFLQPRAL |
| | | | | | | GQ |
| | | | | | | GGAR |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5385 | 0.0156 | 18 | 3606 | 3613 | LMALGPIR |
| 870.5229 | 870.5385 | 0.0156 | 18 | 3606 | 3613 | LMALGPIR |
| 879.4935 | 879.4153 | −0.0782 | −89 | 1874 | 1881 | FVTISGQK |
| 880.441 | 880.4396 | −0.0014 | −2 | 2240 | 2246 | DFTELQK |
| 928.4669 | 928.4629 | −0.004 | −4 | 3522 | 3528 | KEVMEHR |
| 1021.5499 | 1021.5333 | −0.0166 | −16 | 2530 | 2537 | QQVQFMLK |
| 1021.5499 | 1021.5333 | −0.0166 | −16 | 2530 | 2537 | QQVQFMLK |
| 1106.5034 | 1106.5583 | 0.0549 | 50 | 1018 | 1026 | LEEEVEACK |
| 1170.6841 | 1170.6443 | −0.0398 | −34 | 3291 | 3300 | VVKAQIQEQK |
| 1187.6201 | 1187.6656 | 0.0455 | 38 | 2736 | 2745 | NCPISAKLER |
| 1187.6201 | 1187.6656 | 0.0455 | 38 | 2736 | 2745 | NCPISAKLER |
| 1199.6896 | 1199.6674 | −0.0222 | −19 | 3628 | 3637 | AFSIDIIRHK |
| 1257.6797 | 1257.6525 | −0.0272 | −22 | 1506 | 1516 | QISEQLNALNK |
| 1257.6797 | 1257.6525 | −0.0272 | −22 | 1506 | 1516 | QISEQLNALNK |
| 1261.694 | 1261.6499 | −0.0441 | −35 | 380 | 389 | LLEVWIEFGR |
| 1287.6791 | 1287.6593 | −0.0198 | −15 | 4532 | 4542 | EKTLLPEDSQK |
| 1320.7271 | 1320.6016 | −0.1255 | −95 | 1870 | 1881 | GDLRFVTISGQK |
| 1406.7386 | 1406.6833 | −0.0553 | −39 | 4517 | 4528 | QPVYDTTIRTGR |
| 1406.7386 | 1406.6833 | −0.0553 | −39 | 4517 | 4528 | QPVYDTTIRTGR |
| 1413.7809 | 1413.8057 | 0.0248 | 18 | 3135 | 3146 | ARQEQLELTLGR |
| 1420.7213 | 1420.6881 | −0.0332 | −23 | 2919 | 2930 | TGSLEEMTQR |
| | | | | | | LR |
| 1425.7156 | 1425.8075 | 0.0919 | 64 | 869 | 880 | NTISVKAVCD |
| | | | | | | YR |
| 1428.7693 | 1428.7153 | −0.054 | −38 | 4922 | 4933 | LNDALDRLEE |
| | | | | | | LK |
| 1465.7281 | 1465.7726 | 0.0445 | 30 | 4298 | 4309 | EETYNQLLDKGR |
| 1465.7316 | 1465.7726 | 0.041 | 28 | 4310 | 4323 | LMLLSRDDSGSGSK |
| 1487.7952 | 1487.7654 | −0.0298 | −20 | 3435 | 3447 | QTTGEEVLLIQEK |
| 1502.873 | 1502.8582 | −0.0148 | −10 | 380 | 391 | LLEVWIEFGRIK |
| 1532.6785 | 1532.7728 | 0.0943 | 62 | 3761 | 3773 | ELNPEEGEMVEEK |
| 1713.8728 | 1713.8539 | −0.0189 | −11 | 3102 | 3116 | HMLEEEGTLDLLGLK |
| 1727.9149 | 1727.8947 | −0.0202 | −12 | 2151 | 2165 | KLLPQAEMFEHLSGK |
| 1794.9636 | 1794.8103 | −0.1533 | −85 | 4976 | 4991 | QEFIDGILASKFPTTK |
| 1838.8412 | 1838.927 | 0.0858 | 47 | 4830 | 4844 | ALIAEHQTFMEEMTR |
| 2186.155 | 2185.9851 | −0.1699 | −78 | 1958 | 1978 | LLSDTVASDPGVLQE |
| | | | | | | QLA |
| | | | | | | TTK |
| 2202.1799 | 2201.9719 | −0.208 | −94 | 2843 | 2861 | MSELRVTLDPVQLESS |
| | | | | | | LLR |
| 2233.1135 | 2233.0076 | −0.1059 | −47 | 2441 | 2460 | EALAGLLVTYPNSQE |
| | | | | | | AEN |
| | | | | | | WK |
| 2233.1135 | 2233.2017 | 0.0882 | 39 | 2441 | 2460 | EALAGLLVTYPNSQE |
| | | | | | | AEN |
| | | | | | | WK |
| 2299.0217 | 2299.144 | 0.1223 | 53 | 3047 | 3067 | EMFSQLADLDDELDG |
| | | | | | | MG |
| | | | | | | AIGR |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5385 | 0.0156 | 18 | 3680 | 3687 | LMALGPIR |
| 870.5229 | 870.5385 | 0.0156 | 18 | 3680 | 3687 | LMALGPIR |
| 879.4935 | 879.4153 | −0.0782 | −89 | 1839 | 1846 | FVTISGQK |
| 880.441 | 880.4396 | −0.0014 | −2 | 2205 | 2211 | DFTELQK |
| 910.4265 | 910.4448 | 0.0183 | 20 | 3420 | 3426 | YSEIQDR |
| 910.4265 | 910.4448 | 0.0183 | 20 | 3420 | 3426 | YSEIQDR |
| 928.4669 | 928.4629 | −0.004 | −4 | 3596 | 3602 | KEVMEHR |
| 1021.5499 | 1021.5333 | −0.0166 | −16 | 2495 | 2502 | QQVQFMLK |
| 1021.5499 | 1021.5333 | −0.0166 | −16 | 2495 | 2502 | QQVQFMLK |
| 1106.5034 | 1106.5583 | 0.0549 | 50 | 983 | 991 | LEEEVEACK |
| 1170.6841 | 1170.6443 | −0.0398 | −34 | 3256 | 3265 | VVKAQIQEQK |
| 1187.6201 | 1187.6656 | 0.0455 | 38 | 2701 | 2710 | NCPISAKLER |
| 1187.6201 | 1187.6656 | 0.0455 | 38 | 2701 | 2710 | NCPISAKLER |
| 1199.6896 | 1199.6674 | −0.0222 | −19 | 3702 | 3711 | AFSIDIIRHK |
| 1257.6797 | 1257.6525 | −0.0272 | −22 | 1471 | 1481 | QISEQLNALNK |
| 1257.6797 | 1257.6525 | −0.0272 | −22 | 1471 | 1481 | QISEQLNALNK |
| 1261.694 | 1261.6499 | −0.0441 | −35 | 345 | 354 | LLEVWIEFGR |
| 1287.6791 | 1287.6593 | −0.0198 | −15 | 4606 | 4616 | EKTLLPEDSQK |
| 1320.7271 | 1320.6016 | −0.1255 | −95 | 1835 | 1846 | GDLRFVTISGQK |
| 1406.7386 | 1406.6833 | −0.0553 | −39 | 4591 | 4602 | QPVYDTTIRTGR |
| 1406.7386 | 1406.6833 | −0.0553 | −39 | 4591 | 4602 | QPVYDTTIRTGR |
| 1413.7809 | 1413.8057 | 0.0248 | 18 | 3100 | 3111 | ARQEQLELTLGR |
| 1420.7213 | 1420.6881 | −0.0332 | −23 | 2884 | 2895 | TGSLEEMTQRLR |
| 1425.7156 | 1425.8075 | 0.0919 | 64 | 834 | 845 | NTISVKAVCDYR |
| 1428.7693 | 1428.7153 | −0.054 | −38 | 4996 | 5007 | LNDALDRLEELK |
| 1465.7281 | 1465.7726 | 0.0445 | 30 | 4372 | 4383 | EETYNQLLDKGR |
| 1465.7316 | 1465.7726 | 0.041 | 28 | 4384 | 4397 | LMLLSRDDSGSGSK |
| 1487.7952 | 1487.7654 | −0.0298 | −20 | 3509 | 3521 | QTTGEEVLLIQEK |
| 1502.873 | 1502.8582 | −0.0148 | −10 | 345 | 356 | LLEVWIEFGRIK |
| 1532.6785 | 1532.7728 | 0.0943 | 62 | 3835 | 3847 | ELNPEEGEMVEEK |
| 1713.8728 | 1713.8539 | −0.0189 | −11 | 3067 | 3081 | HMLEEEGTLDLLGLK |
| 1727.9149 | 1727.8947 | −0.0202 | −12 | 2116 | 2130 | KLLPQAEMFEHLSGK |
| 1794.9636 | 1794.8103 | −0.1533 | −85 | 5050 | 5065 | QEFIDGILASKFPTTK |
| 1838.8412 | 1838.927 | 0.0858 | 47 | 4904 | 4918 | ALIAEHQTFMEEMTR |
| 2186.155 | 2185.9851 | −0.1699 | −78 | 1923 | 1943 | LLSDTVASDPGVLQE |
| | | | | | | QLA |
| | | | | | | TTK |
| 2202.1799 | 2201.9719 | −0.208 | −94 | 2808 | 2826 | MSELRVTLDPVQLESS |
| | | | | | | LLR |
| 2233.1135 | 2233.0076 | −0.1059 | −47 | 2406 | 2425 | EALAGLLVTYPNSQE |
| | | | | | | AEN |
| | | | | | | WK |
| 2233.1135 | 2233.2017 | 0.0882 | 39 | 2406 | 2425 | EALAGLLVTYPNSQE |
| | | | | | | AEN |
| | | | | | | WK |
| 2299.0217 | 2299.144 | 0.1223 | 53 | 3012 | 3032 | EMFSQLADLDDELDG |
| | | | | | | MG |
| | | | | | | AIGR |

Peptide Information

| | Obsrv. | | | Start | End | |
|---|---|---|---|---|---|---|
| Calc. Mass | Mass | ±da | ±ppm | Seq. | Seq. | Sequence |
| 856.525 | 856.5223 | −0.0027 | −3 | 1408 | 1414 | QLNLLQK |
| 879.4683 | 879.4153 | −0.053 | −60 | 1654 | 1660 | RFSNIDK |
| 880.4774 | 880.4396 | −0.0378 | −43 | 1204 | 1211 | FALTAETK |
| 896.4407 | 896.4399 | −0.0008 | −1 | 747 | 753 | RNFSNMK |
| 910.488 | 910.4448 | −0.0432 | −47 | 1702 | 1709 | SLTGYLEK |
| 910.488 | 910.4448 | −0.0432 | −47 | 1702 | 1709 | SLTGYLEK |
| 912.4573 | 912.4597 | 0.0024 | 3 | 285 | 291 | AELEHWK |
| 928.5403 | 928.4629 | −0.0774 | −83 | 4440 | 4446 | IPAWWKK |
| 985.5941 | 985.582 | −0.0121 | −12 | 2503 | 2509 | RLELWLR |
| 985.5941 | 985.582 | −0.0121 | −12 | 2503 | 2509 | RLELWLR |
| 1005.5363 | 1005.6074 | 0.0711 | 71 | 820 | 827 | VNDLIEFR |
| 1021.4805 | 1021.5333 | 0.0528 | 52 | 2103 | 2111 | SVAMMVPDR |
| 1021.4805 | 1021.5333 | 0.0528 | 52 | 2103 | 2111 | SVAMMVPDR |
| 1106.5411 | 1106.5583 | 0.0172 | 16 | 326 | 333 | TWREMDIR |
| 1187.6816 | 1187.6656 | −0.016 | −13 | 4549 | 4558 | NMKLIESKPK |
| 1187.6816 | 1187.6656 | −0.016 | −13 | 4549 | 4558 | NMKLIESKPK |
| 1199.6995 | 1199.6674 | −0.0321 | −27 | 2585 | 2596 | AVLLIGEQGTAK |
| 1257.7566 | 1257.6525 | −0.1041 | −83 | 167 | 177 | LLSDIFIPALR |
| 1257.7566 | 1257.6525 | −0.1041 | −83 | 167 | 177 | LLSDIFIPALR |
| 1261.6212 | 1261.6499 | 0.0287 | 23 | 1299 | 1308 | VDTLHYAWEK |
| 1271.6553 | 1271.6659 | 0.0106 | 8 | 3711 | 3721 | TSIIDFTVTMK |
| 1332.7369 | 1332.6146 | −0.1223 | −92 | 3210 | 3222 | LKEASESVAALSK |
| 1413.8577 | 1413.8057 | −0.052 | −37 | 166 | 177 | RLLSDIFIPALR |
| 1428.7482 | 1428.7153 | −0.0329 | −23 | 3698 | 3710 | LPNPAYTPEISAR |
| 1502.9153 | 1502.8582 | −0.0571 | −38 | 1119 | 1132 | LVSVLSTIINSTKK |
| 1794.7972 | 1794.8103 | 0.0131 | 7 | 748 | 761 | NFSNMKMMLAEYQR |
| 1838.8668 | 1838.927 | 0.0602 | 33 | 3501 | 3515 | ERWTEQSQEFAAQTK |
| 2266.176 | 2266.0767 | −0.0993 | −44 | 957 | 975 | ELLSHFNHQNMDALL |
| | | | | | | KVTR |

Peptide Information

| | Obsrv. | | | Start | End | |
|---|---|---|---|---|---|---|
| Calc. Mass | Mass | ±da | ±ppm | Seq. | Seq. | Sequence |
| 870.5043 | 870.5385 | 0.0342 | 39 | 2 | 9 | EAALTLPR |
| 870.5043 | 870.5385 | 0.0342 | 39 | 2 | 9 | EAALTLPR |

Peptide Information

| | | | | Start | End | |
|---|---|---|---|---|---|---|
| Calc. Mass | Obsrv. Mass | ±da | ±ppm | Seq. | Seq. | Sequence |
| 924.4897 | 924.4626 | −0.0271 | −29 | 385 | 392 | EILNNHGK |
| 985.5425 | 985.582 | 0.0395 | 40 | 157 | 166 | DAAPGASKLR |
| 985.5425 | 985.582 | 0.0395 | 40 | 157 | 166 | DAAPGASKLR |
| 1021.6153 | 1021.5333 | −0.082 | −80 | 188 | 196 | GVVDHLLLR |
| 1021.6153 | 1021.5333 | −0.082 | −80 | 188 | 196 | GVVDHLLLR |
| 1254.6161 | 1254.6615 | 0.0454 | 36 | 2 | 11 | QPWHGKAMQR |
| 1257.5422 | 1257.6525 | 0.1103 | 88 | 496 | 505 | NNEFPVFDEF |
| 1257.5422 | 1257.6525 | 0.1103 | 88 | 496 | 505 | NNEFPVFDEF |
| 1271.7206 | 1271.6659 | −0.0547 | −43 | 303 | 315 | GGSPAVTLLISEK |
| 1287.6652 | 1287.6593 | −0.0059 | −5 | 12 | 25 | ASEAGATAPKASAR |
| 1413.6719 | 1413.8057 | 0.1338 | 95 | 220 | 231 | ISAPNEFDVMFK |
| 1479.7472 | 1479.7794 | 0.0322 | 22 | 174 | 187 | LSRDDISTAAGMVK |

Peptide Information

| | | | | Start | End | |
|---|---|---|---|---|---|---|
| Calc. Mass | Obsrv. Mass | ±da | ±ppm | Seq. | Seq. | Sequence |
| 879.3876 | 879.4153 | 0.0277 | 31 | 132 | 138 | DDVDMLR |
| 880.4523 | 880.4396 | −0.0127 | −14 | 206 | 212 | DYDALRK |
| 924.4421 | 924.4626 | 0.0205 | 22 | 1766 | 1772 | LEQEYSR |
| 985.5537 | 985.582 | 0.0283 | 29 | 139 | 146 | RENGQLLR |
| 985.5537 | 985.582 | 0.0283 | 29 | 139 | 146 | RENGQLLR |
| 1187.5917 | 1187.6656 | 0.0739 | 62 | 712 | 722 | AHGPEVQAHNK |
| 1187.5917 | 1187.6656 | 0.0739 | 62 | 712 | 722 | AHGPEVQAHNK |
| 1261.6205 | 1261.6499 | 0.0294 | 23 | 358 | 368 | KAANEEMEALR |
| 1406.7526 | 1406.6833 | −0.0693 | −49 | 1495 | 1506 | LADVEQELSFKK |
| 1406.7526 | 1406.6833 | −0.0693 | −49 | 1495 | 1506 | LADVEQELSFKK |
| 1420.7026 | 1420.6881 | −0.0145 | −10 | 1562 | 1575 | DDNSATKTLSAAAR |
| 1487.8315 | 1487.7654 | −0.0661 | −44 | 339 | 351 | LQTEVELAESKLK |
| 1502.7632 | 1502.8582 | 0.095 | 63 | 359 | 371 | AANEEMEALRQIK |
| 1727.9539 | 1727.8947 | −0.0592 | −34 | 1243 | 1259 | VQKGSEPLGISIVSGEK |

Peptide Information

| | | | | Start | End | |
|---|---|---|---|---|---|---|
| Calc. Mass | Obsrv. Mass | ±da | ±ppm | Seq. | Seq. | Sequence |
| 870.5043 | 870.5385 | 0.0342 | 39 | 7 | 14 | DLNPGVKK |
| 870.5043 | 870.5385 | 0.0342 | 39 | 7 | 14 | DLNPGVKK |
| 1106.5623 | 1106.5583 | −0.004 | −4 | 15 | 24 | MSLGQLQSAR |
| 1420.6063 | 1420.6881 | 0.0818 | 58 | 33 | 44 | GTCSGFEPHSWR |
| 2265.9951 | 2266.0767 | 0.0816 | 36 | 25 | 44 | GVACLGCKGTCSGFEPH |

Peptide Information

Tax_Id = 9606 Gene_Symbol = CEP250 Isoform
1 of Centrosome-associated protein CEP250

| | Obsrv. | | | Start | End | |
|---|---|---|---|---|---|---|
| Calc. Mass | Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADVVNKALR |
| 1065.5067 | 1065.5122 | 0.0055 | 5 | 883 | 890 | EKMELEMR |
| 1232.6117 | 1232.6262 | 0.0145 | 12 | 1390 | 1399 | LKNEEVESER |
| 1235.5837 | 1235.5809 | −0.0028 | −2 | 68 | 76 | SWCQELEKR |
| 1257.691 | 1257.6628 | −0.0282 | −22 | 1667 | 1676 | IQVLEDQRTR |
| 1257.705 | 1257.6628 | −0.0422 | −34 | 601 | 612 | LSALNEALALDK |
| 1323.728 | 1323.6946 | −0.0334 | −25 | 172 | 182 | GEHGRLLSLWR |
| 1425.7081 | 1425.8451 | 0.137 | 96 | 2371 | 2382 | QDYITRSAQTSR |
| 1425.7081 | 1425.8451 | 0.137 | 96 | 2371 | 2382 | QDYITRSAQTSR |
| 1487.77 | 1487.8041 | 0.0341 | 23 | 753 | 766 | QDLAEQLQGLSSAK |
| 1497.8384 | 1497.7552 | −0.0832 | −56 | 1881 | 1893 | RVQALEEVLGDLR |
| 1532.785 | 1532.8186 | 0.0336 | 22 | 1698 | 1709 | ELTTQRQLMQER |
| 1579.7819 | 1579.8885 | 0.1066 | 67 | 522 | 534 | ERLQEMLMGLEAK |
| 1708.9089 | 1708.9078 | −0.0011 | −1 | 2292 | 2305 | HNVQLRSTLEQVER |
| | 1708.9078 | | | | | |
| 1713.8767 | 1713.9175 | 0.0408 | 24 | 492 | 507 | VNVELQLQGDSAQGQK |

Peptide Information

Tax_Id = 9606 Gene_Symbol = CEP250 Isoform
2 of Centrosome-associated protein CEP250

| | | | | Start | End | |
|---|---|---|---|---|---|---|
| Calc. Mass | Obsrv. Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADVVNKALR |
| 1232.6117 | 1232.6262 | 0.0145 | 12 | 1334 | 1343 | LKNEEVESER |
| 1235.5837 | 1235.5809 | −0.0028 | −2 | 68 | 76 | SWCQELEKR |
| 1257.691 | 1257.6628 | −0.0282 | −22 | 1611 | 1620 | IQVLEDQRTR |
| 1257.705 | 1257.6628 | −0.0422 | −34 | 601 | 612 | LSALNEALALDK |
| 1323.728 | 1323.6946 | −0.0334 | −25 | 172 | 182 | GEHGRLLSLWR |
| 1425.7081 | 1425.8451 | 0.137 | 96 | 2315 | 2326 | QDYITRSAQTSR |
| 1425.7081 | 1425.8451 | 0.137 | 96 | 2315 | 2326 | QDYITRSAQTSR |
| 1487.77 | 1487.8041 | 0.0341 | 23 | 753 | 766 | QDLAEQLQGLSSAK |
| 1497.8384 | 1497.7552 | −0.0832 | −56 | 1825 | 1837 | RVQALEEVLGDLR |
| 1532.785 | 1532.8186 | 0.0336 | 22 | 1642 | 1653 | ELTTQRQLMQER |
| 1579.7819 | 1579.8885 | 0.1066 | 67 | 522 | 534 | ERLQEMLMGLEAK |
| 1708.9089 | 1708.9078 | −0.0011 | −1 | 2236 | 2249 | HNVQLRSTLEQVER |
| 1713.8767 | 1713.9175 | 0.0408 | 24 | 492 | 507 | VNVELQLQGDSAQG |
| | | | | | | QK |

Peptide Information

Tax_Id = 9606 Gene_Symbol = CEP250
Uncharacterized protein

| | | | | Start | End | |
|---|---|---|---|---|---|---|
| Calc. Mass | Obsrv. Mass | ±da | ±ppm | Seq. | Seq. | Sequence |
| 985.5537 | 985.5696 | 0.0159 | 16 | 399 | 406 | RQAVQDLR |
| 985.5789 | 985.5696 | −0.0093 | −9 | 127 | 135 | ADVVNKALR |
| 1235.5837 | 1235.5809 | −0.0028 | −2 | 68 | 76 | SWCQELEKR |
| 1323.728 | 1323.6946 | −0.0334 | −25 | 172 | 182 | GEHGRLLSLWR |
| 1713.8767 | 1713.9175 | 0.0408 | 24 | 492 | 507 | VNVELQLQGDSAQGQK |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5196 | −0.0033 | −4 | 3736 | 3743 | LMALGPIR |
| 880.441 | 880.4194 | −0.0216 | −25 | 2240 | 2246 | DFTELQK |
| 910.4265 | 910.4317 | 0.0052 | 6 | 3476 | 3482 | YSEIQDR |
| 910.4265 | 910.4317 | 0.0052 | 6 | 3476 | 3482 | YSEIQDR |
| 928.4669 | 928.4491 | −0.0178 | −19 | 3652 | 3658 | KEVMEHR |
| 1021.5499 | 1021.5277 | −0.0222 | −22 | 2551 | 2558 | QQVQFMLK |
| 1021.5499 | 1021.5277 | −0.0222 | −22 | 2551 | 2558 | QQVQFMLK |
| 1170.5902 | 1170.6512 | 0.061 | 52 | 2820 | 2828 | NHWEELSKK |
| 1170.6841 | 1170.6512 | −0.0329 | −28 | 3312 | 3321 | VVKAQIQEQK |
| 1187.6201 | 1187.681 | 0.0609 | 51 | 2757 | 2766 | NCPISAKLER |
| 1187.6201 | 1187.681 | 0.0609 | 51 | 2757 | 2766 | NCPISAKLER |
| 1232.6117 | 1232.6262 | 0.0145 | 12 | 2659 | 2668 | QQLEETSEIR |
| 1235.6378 | 1235.5809 | −0.0569 | −46 | 1058 | 1066 | LRLEEYEQR |
| 1257.6797 | 1257.6628 | −0.0169 | −13 | 1506 | 1516 | QISEQLNALNK |
| 1257.6797 | 1257.6628 | −0.0169 | −13 | 1506 | 1516 | QISEQLNALNK |
| 1261.694 | 1261.6696 | −0.0244 | −19 | 380 | 389 | LLEVWIEFGR |
| 1320.7271 | 1320.6184 | −0.1087 | −82 | 1870 | 1881 | GDLRFVTISGQK |
| 1323.7896 | 1323.6946 | −0.095 | −72 | 3670 | 3680 | ALLELVPWRAR |
| 1406.7386 | 1406.7107 | −0.0279 | −20 | 4647 | 4658 | QPVYDTTIRTGR |
| 1406.7386 | 1406.7107 | −0.0279 | −20 | 4647 | 4658 | QPVYDTTIRTGR |
| 1413.7809 | 1413.8478 | 0.0669 | 47 | 3156 | 3167 | ARQEQLELTLGR |
| 1420.7213 | 1420.7368 | 0.0155 | 11 | 2940 | 2951 | TGSLEEMTQRLR |
| 1425.7156 | 1425.8451 | 0.1295 | 91 | 869 | 880 | NTISVKAVCDYR |
| 1425.7156 | 1425.8451 | 0.1295 | 91 | 869 | 880 | NTISVKAVCDYR |
| 1428.7693 | 1428.7944 | 0.0251 | 18 | 5052 | 5063 | LNDALDRLEELK |
| 1465.7281 | 1465.8011 | 0.073 | 50 | 4428 | 4439 | EETYNQLLDKGR |
| 1465.7316 | 1465.8011 | 0.0695 | 47 | 4440 | 4453 | LMLLSRDDSGSG |
| 1487.7952 | 1487.8041 | 0.0089 | 6 | 3565 | 3577 | QTTGEEVLLIQEK |
| 1502.873 | 1502.8989 | 0.0259 | 17 | 380 | 391 | LLEVWIEFGRIK |
| 1532.6785 | 1532.8186 | 0.1401 | 91 | 3891 | 3903 | ELNPEEGEMVEE |
| 1708.8389 | 1708.9078 | 0.0689 | 40 | 3681 | 3695 | EGLDKLVSDANEQY |
| 1713.8728 | 1713.9175 | 0.0447 | 26 | 3123 | 3137 | HMLEEEGTLDLLGLK |
| 1727.9149 | 1727.9177 | 0.0028 | 2 | 2151 | 2165 | KLLPQAEMFEHLSGK |
| 1950.9412 | 1951.001 | 0.0598 | 31 | 4960 | 4975 | ALIAEHQTFMEEMTR |
| 1966.9362 | 1966.9954 | 0.0592 | 30 | 4960 | 4975 | ALIAEHQTFMEEMTR |
| 2185.0693 | 2185.1575 | 0.0882 | 40 | 3885 | 3903 | IGPQLKELNPEEGEMK |
| 2186.155 | 2186.0022 | −0.1528 | −70 | 1958 | 1978 | LLSDTVASDPGVLQEA |
| | | | | | | TTK |
| 2186.1851 | 2186.1931 | 0.008 | 4 | 2864 | 2882 | MSELRVTLDPVQLESR |
| 2200.0632 | 2200.0994 | 0.0362 | 16 | 4015 | 4031 | EIQDKLDQMVFFWED |
| 2202.1799 | 2202.2275 | 0.0476 | 22 | 2864 | 2882 | MSELRVTLDPVQLESR |
| 2212.2183 | 2212.3137 | 0.0954 | 43 | 3558 | 3577 | NGQALLKQTTGEEVLQ |
| | | | | | | EK |

Peptide Information

Tax_Id = 9606 Gene_Symbol = MACF1 Isoform
3 of Microtubule-actin cross-linking factor 1,
isoforms 1/2/3/5

| | Obsrv. | | | Start | End | |
|---|---|---|---|---|---|---|
| Calc. Mass | Mass | ±da | ±ppm | Seq. | Seq. | Sequence |
| 870.5229 | 870.5196 | −0.0033 | −4 | 3680 | 3687 | LMALGPIR |
| 880.441 | 880.4194 | −0.0216 | −25 | 2205 | 2211 | DFTELQK |
| 910.4265 | 910.4317 | 0.0052 | 6 | 3420 | 3426 | YSEIQDR |
| 910.4265 | 910.4317 | 0.0052 | 6 | 3420 | 3426 | YSEIQDR |
| 928.4669 | 928.4491 | −0.0178 | −19 | 3596 | 3602 | KEVMEHR |
| 1021.5499 | 1021.5277 | −0.0222 | −22 | 2495 | 2502 | QQVQFMLK |
| 1021.5499 | 1021.5277 | −0.0222 | −22 | 2495 | 2502 | QQVQFMLK |
| 1170.5902 | 1170.6512 | 0.061 | 52 | 2764 | 2772 | NHWEELSKK |
| 1170.6841 | 1170.6512 | −0.0329 | −28 | 3256 | 3265 | VVKAQIQEQK |
| 1187.6201 | 1187.681 | 0.0609 | 51 | 2701 | 2710 | NCPISAKLER |
| 1187.6201 | 1187.681 | 0.0609 | 51 | 2701 | 2710 | NCPISAKLER |
| 1232.6117 | 1232.6262 | 0.0145 | 12 | 2603 | 2612 | QQLEETSEIR |
| 1235.6378 | 1235.5809 | −0.0569 | −46 | 1023 | 1031 | LRLEEYEQR |
| 1257.6797 | 1257.6628 | −0.0169 | −13 | 1471 | 1481 | QISEQLNALNK |
| 1257.6797 | 1257.6628 | −0.0169 | −13 | 1471 | 1481 | QISEQLNALN |
| 1261.694 | 1261.6696 | −0.0244 | −19 | 345 | 354 | LLEVWIEFGR |
| 1320.7271 | 1320.6184 | −0.1087 | −82 | 1835 | 1846 | GDLRFVTISGQK |
| 1323.7896 | 1323.6946 | −0.095 | −72 | 3614 | 3624 | ALLELVPWRAR |
| 1406.7386 | 1406.7107 | −0.0279 | −20 | 4591 | 4602 | QPVYDTTIRTGR |
| 1406.7386 | 1406.7107 | −0.0279 | −20 | 4591 | 4602 | QPVYDTTIRTGR |
| 1413.7809 | 1413.8478 | 0.0669 | 47 | 3100 | 3111 | ARQEQLELTLGR |
| 1420.7213 | 1420.7368 | 0.0155 | 11 | 2884 | 2895 | TGSLEEMTQRLR |
| 1425.7156 | 1425.8451 | 0.1295 | 91 | 834 | 845 | NTISVKAVCDYR |
| 1425.7156 | 1425.8451 | 0.1295 | 91 | 834 | 845 | NTISVKAVCDYR |
| 1428.7693 | 1428.7944 | 0.0251 | 18 | 4996 | 5007 | LNDALDRLEELK |
| 1465.7281 | 1465.8011 | 0.073 | 50 | 4372 | 4383 | EETYNQLLDKGR |
| 1465.7316 | 1465.8011 | 0.0695 | 47 | 4384 | 4397 | LMLLSRDDSGSG |
| | | | | | | SK |
| 1487.7952 | 1487.8041 | 0.0089 | 6 | 3509 | 3521 | QTTGEEVLLIQEK |
| 1502.873 | 1502.8989 | 0.0259 | 17 | 345 | 356 | LLEVWIEFGRIK |
| 1532.6785 | 1532.8186 | 0.1401 | 91 | 3835 | 3847 | ELNPEEGEMVEEK |
| 1708.8389 | 1708.9078 | 0.0689 | 40 | 3625 | 3639 | EGLDKLVSDANEQYK |
| 1713.8728 | 1713.9175 | 0.0447 | 26 | 3067 | 3081 | HMLEEEGTLDLLGLK |
| 1727.9149 | 1727.9177 | 0.0028 | 2 | 2116 | 2130 | KLLPQAEMFEHLSGK |
| 1950.9412 | 1951.001 | 0.0598 | 31 | 4904 | 4919 | ALIAEHQTFMEEMTRK |
| 1966.9362 | 1966.9954 | 0.0592 | 30 | 4904 | 4919 | ALIAEHQTFMEEMTRK |
| 2185.0693 | 2185.1575 | 0.0882 | 40 | 3829 | 3847 | IGPQLKELNPEEGEM |
| | | | | | | VEEK |
| 2186.155 | 2186.0022 | −0.1528 | −70 | 1923 | 1943 | LLSDTVASDPGVLQE |
| | | | | | | QLA |
| | | | | | | TTK |
| 2186.1851 | 2186.1931 | 0.008 | 4 | 2808 | 2826 | MSELRVTLDPVQLES |
| | | | | | | SLLR |
| 2200.0632 | 2200.0994 | 0.0362 | 16 | 3959 | 3975 | EIQDKLDQMVFFWED |
| | | | | | | IK |
| 2202.1799 | 2202.2275 | 0.0476 | 22 | 2808 | 2826 | MSELRVTLDPVQLES |
| | | | | | | SLLR |
| 2212.2183 | 2212.3137 | 0.0954 | 43 | 3502 | 3521 | NGQALLKQTTGEEVL |
| | | | | | | LIQ |
| | | | | | | EK |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5196 | −0.0033 | −4 | 3606 | 3613 | LMALGPIR |
| 880.441 | 880.4194 | −0.0216 | −25 | 2240 | 2246 | DFTELQK |
| 928.4669 | 928.4491 | −0.0178 | −19 | 3522 | 3528 | KEVMEHR |
| 1021.5499 | 1021.5277 | −0.0222 | −22 | 2530 | 2537 | QQVQFMLK |
| 1021.5499 | 1021.5277 | −0.0222 | −22 | 2530 | 2537 | QQVQFMLK |
| 1170.5902 | 1170.6512 | 0.061 | 52 | 2799 | 2807 | NHWEELSKK |
| 1170.6841 | 1170.6512 | −0.0329 | −28 | 3291 | 3300 | VVKAQIQEQK |
| 1187.6201 | 1187.681 | 0.0609 | 51 | 2736 | 2745 | NCPISAKLER |
| 1187.6201 | 1187.681 | 0.0609 | 51 | 2736 | 2745 | NCPISAKLER |
| 1232.6117 | 1232.6262 | 0.0145 | 12 | 2638 | 2647 | QQLEETSEIR |
| 1235.6378 | 1235.5809 | −0.0569 | −46 | 1058 | 1066 | LRLEEYEQR |
| 1257.6797 | 1257.6628 | −0.0169 | −13 | 1506 | 1516 | QISEQLNALNK |
| 1257.6797 | 1257.6628 | −0.0169 | −13 | 1506 | 1516 | QISEQLNALNK |
| 1261.694 | 1261.6696 | −0.0244 | −19 | 380 | 389 | LLEVWIEFGR |
| 1320.7271 | 1320.6184 | −0.1087 | −82 | 1870 | 1881 | GDLRFVTISGQK |
| 1323.7896 | 1323.6946 | −0.095 | −72 | 3540 | 3550 | ALLELVPWRAR |
| 1406.7386 | 1406.7107 | −0.0279 | −20 | 4517 | 4528 | QPVYDTTIRTGR |
| 1406.7386 | 1406.7107 | −0.0279 | −20 | 4517 | 4528 | QPVYDTTIRTGR |
| 1413.7809 | 1413.8478 | 0.0669 | 47 | 3135 | 3146 | ARQEQLELTLGR |
| 1420.7213 | 1420.7368 | 0.0155 | 11 | 2919 | 2930 | TGSLEEMTQRLR |
| 1425.7156 | 1425.8451 | 0.1295 | 91 | 869 | 880 | NTISVKAVCDYR |
| 1425.7156 | 1425.8451 | 0.1295 | 91 | 869 | 880 | NTISVKAVCDYR |
| 1428.7693 | 1428.7944 | 0.0251 | 18 | 4922 | 4933 | LNDALDRLEELK |
| 1465.7281 | 1465.8011 | 0.073 | 50 | 4298 | 4309 | EETYNQLLDKGR |
| 1465.7316 | 1465.8011 | 0.0695 | 47 | 4310 | 4323 | LMLLSRDDSGSG |
| | | | | | | SK |
| 1487.7952 | 1487.8041 | 0.008 | 96 | 3435 | 3447 | QTTGEEVLLIQEK |
| 1502.873 | 1502.8989 | 0.0259 | 17 | 380 | 391 | LLEVWIEFGRIK |
| 1532.6785 | 1532.8186 | 0.1401 | 91 | 3761 | 3773 | ELNPEEGEMVEEK |
| 1708.8389 | 1708.9078 | 0.0689 | 40 | 3551 | 3565 | EGLDKLVSDANEQYK |
| 1713.8728 | 1713.9175 | 0.0447 | 26 | 3102 | 3116 | HMLEEEGTLDLLGLK |
| 1727.9149 | 1727.9177 | 0.0028 | 2 | 2151 | 2165 | KLLPQAEMFEHLSGK |
| 1950.9412 | 1951.001 | 0.0598 | 31 | 4830 | 4845 | ALIAEHQTFMEEMTRK |
| 1966.9362 | 1966.9954 | 0.0592 | 30 | 4830 | 4845 | ALIAEHQTFMEEMTRK |
| 2185.0693 | 2185.1575 | 0.0882 | 40 | 3755 | 3773 | IGPQLKELNPEEGEM |
| | | | | | | VEEK |
| 2186.155 | 2186.0022 | −0.1528 | −70 | 1958 | 1978 | LLSDTVASDPGVLQE |
| | | | | | | QL |
| | | | | | | A TTK |
| 2186.1851 | 2186.1931 | 0.008 | 4 | 2843 | 2861 | MSELRVTLDPVQLES |
| | | | | | | SLLR |
| 2200.0632 | 2200.0994 | 0.0362 | 16 | 3885 | 3901 | EIQDKLDQMVFFWE |
| | | | | | | DIK |
| 2202.1799 | 2202.2275 | 0.0476 | 22 | 2843 | 2861 | MSELRVTLDPVQLES |
| | | | | | | SLL |
| | 2202.2275 | | | | | |
| 2212.2183 | 2212.3137 | 0.0954 | 43 | 3428 | 3447 | R |
| | | | | | | NGQALLKQTTGEEVL |
| | | | | | | LI |
| | | | | | | Q EK |

Peptide Information

Tax_Id = 9606 Gene_Symbol = COL6A3
collagen alpha-3(VI) chain isoform 2 precursor

| | Obsrv. | | | Start | End | |
|---|---|---|---|---|---|---|
| Calc. Mass | Mass | ±da | ±ppm | Seq. | Seq. | Sequence |
| 896.4625 | 896.4256 | −0.0369 | −41 | 246 | 252 | TNFPYVR |
| 910.5104 | 910.4317 | −0.0787 | −86 | 293 | 300 | SDILGHLR |
| 910.5104 | 910.4317 | −0.0787 | −86 | 293 | 300 | SDILGHLR |
| 1187.7008 | 1187.681 | −0.0198 | −17 | 915 | 924 | NIFKRPLGSR |
| 1187.7008 | 1187.681 | −0.0198 | −17 | 915 | 924 | NIFKRPLGSR |
| 1320.7311 | 1320.6184 | −0.1127 | −85 | 558 | 569 | QSGVVPFIFQAK |
| 1420.7948 | 1420.7368 | −0.058 | −41 | 635 | 646 | SGFPLLKEFVQR |
| 1425.7445 | 1425.8451 | 0.1006 | 71 | 219 | 231 | TLSGTPEVHSNKR |
| 1425.7445 | 1425.8451 | 0.1006 | 71 | 219 | 231 | TLSGTPEVHSNKR |
| 1579.9781 | 1579.8885 | −0.0896 | −57 | 138 | 153 | AAEGIPKLLVLITGGK |
| 1950.9518 | 1951.001 | 0.0492 | 25 | 1018 | 1036 | YPPPGEMGASEVLLG |
| | | | | | | AF |
| | | | | | | SI |
| 2185.1323 | 2185.1575 | 0.0252 | 12 | 107 | 126 | KMKPLDGSALYTGS |
| | | | | | | ALD |
| | | | | | | FVR |
| 2200.2449 | 2200.0994 | −0.1455 | −66 | 524 | 544 | SAGSRIEDGVLQFLV |
| | | | | | | LLV |
| | | | | | | AGR |
| 2202.1919 | 2202.2275 | 0.0356 | 16 | 549 | 569 | VDGPASNLKQSGVVP |
| | | | | | | FIF |
| | | | | | | QAK |

Peptide Information

Tax_Id = 9606 Gene_Symbol = FGF4 Fibroblast
growth factor 4

| | Obsrv. | | | Start | End | |
|---|---|---|---|---|---|---|
| Calc. Mass | Mass | ±da | ±ppm | Seq. | Seq. | Sequence |
| 1005.5146 | 1005.6021 | 0.0875 | 87 | 190 | 198 | GNRVSPTMK |
| 1257.6686 | 1257.6628 | −0.0058 | −5 | 113 | 123 | DSLLELSPVER |
| 1257.6686 | 1257.6628 | −0.0058 | −5 | 113 | 123 | DSLLELSPVER |
| 1425.7559 | 1425.8451 | 0.0892 | 63 | 174 | 186 | YPGMFIALSKNGK |
| 1425.7559 | 1425.8451 | 0.0892 | 63 | 174 | 186 | YPGMFIALSKNGK |
| 2186.1289 | 2186.0022 | −0.1267 | −58 | 85 | 103 | RLYCNVGIGFHLQALP |
| | | | | | | DGR |
| 2186.1289 | 2186.1931 | 0.0642 | 29 | 85 | 103 | RLYCNVGIGFHLQALP |
| | | | | | | DGR |

Peptide Information

Tax_Id = 9606 Gene_Symbol = FBXO41 F-box only protein 41

| | Obsrv. | | | Start | End | |
|---|---|---|---|---|---|---|
| Calc. Mass | Mass | ±da | ±ppm | Seq. | Seq. | Sequence |
| 982.4914 | 982.4271 | −0.0643 | −65 | 907 | 914 | LFEDMVTK |
| 1465.7329 | 1465.8011 | 0.0682 | 47 | 30 | 43 | MAGASPAVPHERAR |
| 1465.7329 | 1465.8011 | 0.0682 | 47 | 30 | 43 | MAGASPAVPHERAR |
| 1579.8513 | 1579.8885 | 0.0372 | 24 | 907 | 919 | LFEDMVTKLQALR |
| 1713.9065 | 1713.9175 | 0.011 | 6 | 808 | 826 | ALGVGGAGCGVQGL |
| | | | | | | ASL |
| | | | | | | AR |
| 2894.479 | 2894.4836 | 0.0046 | 2 | 2 | 27 | TTGLSDQQVVCDLDH |
| | | | | | | RA |
| | | | | | | VEALLQAVR |

Peptide Information

Tax_Id = 9606 Gene_Symbol = MCM8
Uncharacterized protein

| | Obsrv. | | | Start | End | |
|---|---|---|---|---|---|---|
| Calc. Mass | Mass | ±da | ±ppm | Seq. | Seq. | Sequence |
| 982.4523 | 982.4271 | −0.0252 | −26 | 1 | 8 | MNGEYRGR |
| 1065.5225 | 1065.5122 | −0.0103 | −10 | 23 | 32 | GGGNFSGKWR |
| 1479.769 | 1479.8186 | 0.0496 | 34 | 48 | 60 | TSEQTPQFLLSTK |
| 2170.1238 | 2170.114 | −0.0098 | −5 | 127 | 146 | ELTEGGEVTNLIPDIA |
| | | | | | | TELR |
| 2185.1467 | 2185.1575 | 0.0108 | 5 | 152 | 170 | TLACMGLAIHQVLTK |
| | | | | | | DLER |
| 2212.1465 | 2212.3137 | 0.1672 | 76 | 147 | 166 | DAPEKTLACMGLAIH |
| | | | | | | QVL |

Peptide Information

Tax_Id = 9606 Gene_Symbol = CEP250 Isoform 1 of
Centrosome-associated protein CEP250

| | Obsrv. | | | Start | End | |
|---|---|---|---|---|---|---|
| Calc. Mass | Mass | ±da | ±ppm | Seq. | Seq. | Sequence |
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1097.4966 | 1097.5127 | 0.0161 | 15 | 883 | 890 | EKMELEMR |
| 1232.6117 | 1232.6179 | 0.0062 | 5 | 1390 | 1399 | LKNEEVESER |
| 1257.691 | 1257.6606 | −0.0304 | −24 | 1667 | 1676 | IQVLEDQRTR |
| 1257.705 | 1257.6606 | −0.0444 | −35 | 601 | 612 | LSALNEALALDK |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1425.7081 | 1425.8483 | 0.1402 | 98 | 2371 | 2382 | QDYITRSAQTSR |
| 1425.7081 | 1425.8483 | 0.1402 | 98 | 2371 | 2382 | QDYITRSAQTSR |
| 1487.77 | 1487.7893 | 0.0193 | 13 | 753 | 766 | QDLAEQLQGLSSAK |
| 1532.785 | 1532.8113 | 0.0263 | 17 | 1698 | 1709 | ELTTQRQLMQER |
| 1579.7819 | 1579.8809 | 0.099 | 63 | 522 | 534 | ERLQEMLMGLEAK |
| 1657.9484 | 1657.8533 | −0.0951 | −57 | 926 | 939 | ERVSLLETLLQTQK |
| 1708.9089 | 1708.9053 | −0.0036 | −2 | 2292 | 2305 | HNVQLRSTLEQVER |
| 1713.8767 | 1713.9238 | 0.0471 | 27 | 492 | 507 | VNVELQLQGDSAQGQK |
| 2092.1001 | 2091.998 | −0.1021 | −49 | 212 | 230 | LSGSLLTCCLRLTVGAQSR |

Peptide Information

Tax_Id = 9606 Gene_Symbol = CEP250 Isoform 2 of
Centrosome-associated protein CEP250

| | Obsrv. | | | Start | End | |
|---|---|---|---|---|---|---|
| Calc. Mass | Mass | ±da | ±ppm | Seq. | Seq. | Sequence |
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1232.6117 | 1232.6179 | 0.0062 | 5 | 1334 | 1343 | LKNEEVESER |
| 1257.691 | 1257.6606 | −0.0304 | −24 | 1611 | 1620 | IQVLEDQRTR |
| 1257.705 | 1257.6606 | −0.0444 | −35 | 601 | 612 | LSALNEALALDK |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1425.7081 | 1425.8483 | 0.1402 | 98 | 2315 | 2326 | QDYITRSAQTSR |
| 1425.7081 | 1425.8483 | 0.1402 | 98 | 2315 | 2326 | QDYITRSAQTSR |
| 1487.77 | 1487.7893 | 0.0193 | 13 | 753 | 766 | QDLAEQLQGLSSAK |
| 1532.785 | 1532.8113 | 0.0263 | 17 | 1642 | 1653 | ELTTQRQLMQER |
| 1579.7819 | 1579.8809 | 0.099 | 63 | 522 | 534 | ERLQEMLMGLEAK |
| 1657.9484 | 1657.8533 | −0.0951 | −57 | 870 | 883 | ERVSLLETLLQTQK |
| 1708.9089 | 1708.9053 | −0.0036 | −2 | 2236 | 2249 | HNVQLRSTLEQVER |
| | 1708.9053 | | | | | |
| 1713.8767 | 1713.9238 | 0.0471 | 27 | 492 | 507 | VNVELQLQGDSAQG |
| | | | | | | QK |
| 2092.1001 | 2091.998 | −0.1021 | −49 | 212 | 230 | LSGSLLTCCLRLTVG |
| | | | | | | AQSR |

Peptide Information

Tax_Id = 9606 Gene_Symbol = CEP250
Uncharacterized protein

| | Obsrv. | | | Start | End | |
|---|---|---|---|---|---|---|
| Calc. Mass | Mass | ±da | ±ppm | Seq. | Seq. | Sequence |
| 985.5537 | 985.5631 | 0.0094 | 10 | 399 | 406 | RQAVQDLR |
| 985.5537 | 985.5631 | 0.0094 | 10 | 399 | 406 | RQAVQDLR |
| 1283.6776 | 1283.6473 | −0.0303 | −24 | 122 | 132 | LHMEKADVVNK |
| 1350.6471 | 1350.7144 | 0.0673 | 50 | 190 | 200 | HFLEMKSATDR |
| 1546.837 | 1546.7799 | −0.0571 | −37 | 524 | 536 | LQSSQLQSCRVLK |
| 1713.8767 | 1713.9238 | 0.0471 | 27 | 492 | 507 | VNVELQLQGDSAQG |
| | | | | | | QK |
| 2092.1001 | 2091.998 | −0.1021 | −49 | 212 | 230 | LSGSLLTCCLRLTVG |
| | | | | | | AQ |

Peptide Information

S R Tax_Id = 9606 Gene_Symbol = MACF1 Isoform 3 of
Microtubule-actin cross-linking factor 1, isoforms 1/2/3/5

| | Obsrv. | | | Start | End | |
|---|---|---|---|---|---|---|
| Calc. Mass | Mass | ±da | ±ppm | Seq. | Seq. | Sequence |
| 842.4519 | 842.4806 | 0.0287 | 34 | 4409 | 4415 | WHVVSSK |
| 870.5229 | 870.511 | −0.0119 | −14 | 3680 | 3687 | LMALGPIR |
| 910.4265 | 910.4239 | −0.0026 | −3 | 3420 | 3426 | YSEIQDR |
| 910.4265 | 910.4239 | −0.0026 | −3 | 3420 | 3426 | YSEIQDR |
| 1021.5499 | 1021.5206 | −0.0293 | −29 | 2495 | 2502 | QQVQFMLK |
| 1021.5499 | 1021.5206 | −0.0293 | −29 | 2495 | 2502 | QQVQFMLK |
| 1170.6841 | 1170.6456 | −0.0385 | −33 | 3256 | 3265 | VVKAQIQEQK |
| 1187.6201 | 1187.673 | 0.0529 | 45 | 2701 | 2710 | NCPISAKLER |
| 1232.6117 | 1232.6179 | 0.0062 | 5 | 2603 | 2612 | QQLEETSEIR |
| 1257.6797 | 1257.6606 | −0.0191 | −15 | 1471 | 1481 | QISEQLNALNK |
| 1257.6797 | 1257.6606 | −0.0191 | −15 | 1471 | 1481 | QISEQLNALNK |
| 1261.694 | 1261.6659 | −0.0281 | −22 | 345 | 354 | LLEVWIEFGR |
| 1320.7271 | 1320.6123 | −0.1148 | −87 | 1835 | 1846 | GDLRFVTISGQK |
| 1406.7386 | 1406.7148 | −0.0238 | −17 | 4591 | 4602 | QPVYDTTIRTGR |
| 1406.7386 | 1406.7148 | −0.0238 | −17 | 4591 | 4602 | QPVYDTTIRTGR |
| | 1406.7148 | | | | | |
| | 2 | | | | | |
| 1420.7213 | 1420.736 | 0.0147 | 10 | 2884 | 2895 | TGSLEEMTQRLR |
| 1425.7156 | 1425.8483 | 0.1327 | 93 | 834 | 845 | NTISVKAVCDYR |
| 1425.7156 | 1425.8483 | 0.1327 | 93 | 834 | 845 | NTISVKAVCDYR |
| 1428.7693 | 1428.7908 | 0.0215 | 15 | 4996 | 5007 | LNDALDRLEELK |
| 1450.6996 | 1450.7076 | 0.008 | 6 | 2100 | 2110 | FEQLCLQQQEK |
| 1465.7281 | 1465.804 | 0.0759 | 52 | 4372 | 4383 | EETYNQLLDKGR |
| 1465.7316 | 1465.804 | 0.0724 | 49 | 4384 | 4397 | LMLLSRDDSGSGSK |
| 1487.7952 | 1487.7893 | −0.0059 | −4 | 3509 | 3521 | QTTGEEVLLIQEK |
| 1502.873 | 1502.896 | 0.023 | 15 | 345 | 356 | LLEVWIEFGRIK |
| 1502.873 | 1502.896 | 0.023 | 15 | 345 | 356 | LLEVWIEFGRIK |
| 1532.6785 | 1532.8113 | 0.1328 | 87 | 3835 | 3847 | ELNPEEGEMVEEK |
| 1546.8727 | 1546.7799 | −0.0928 | −60 | 3982 | 3994 | EIKFLDVLELAEK |
| 1707.7603 | 1707.8604 | 0.1001 | 59 | 854 | 867 | NDECVLEDNSQRTK |
| 1708.8389 | 1708.9053 | 0.0664 | 39 | 3625 | 3639 | EGLDKLVSDANEQYK |
| 1713.8728 | 1713.9238 | 0.051 | 30 | 3067 | 3081 | HMLEEEGTLDLLGLK |
| 1727.9149 | 1727.9309 | 0.016 | 9 | 2116 | 2130 | KLLPQAEMFEHLSGK |
| 1813.8942 | 1813.937 | 0.0428 | 24 | 3964 | 3977 | LDQMVFFWEDIKAR |
| 1950.9412 | 1951.0114 | 0.0702 | 36 | 4904 | 4919 | ALIAEHQTFMEEMT |
| | | | | | | RK |
| 1966.9362 | 1967.0013 | 0.0651 | 33 | 4904 | 4919 | ALIAEHQTFMEEMT |
| | | | | | | RK |
| 2091.9805 | 2091.998 | 0.0175 | 8 | 461 | 476 | DENYYQLEELAFRV |
| | | | | | | MR |
| 2186.155 | 2185.9929 | −0.1621 | −74 | 1923 | 1943 | LLSDTVASDPGVLQE |
| | | | | | | QLA |
| | | | | | | TTK |
| 2186.1851 | 2186.1921 | 0.007 | 3 | 2808 | 2826 | MSELRVTLDPVQLES |
| | | | | | | SLLR |
| 2211.1301 | 2211.2874 | 0.1573 | 71 | 5151 | 5170 | STVMVRVGGGWMA |
| | | | | | | LDE |
| | | | | | | FLVK |
| 2501.2268 | 2501.4001 | 0.1733 | 69 | 1304 | 1323 | FSQQYSTIVKDYELQ |
| | | | | | | LMT |
| | | | | | | YK |

Peptide Information

Tax_Id = 9606 Gene Symbol = ENOPH1
Uncharacterized protei

| | Obsrv. | | | Start | End | |
|---|---|---|---|---|---|---|
| Calc. Mass | Mass | ±da | ±ppm | Seq. | Seq. | Sequence |
| 1320.6947 | 1320.6123 | −0.0824 | −62 | 129 | 140 | AEFFADVVPAVR |
| 1479.7729 | 1479.8213 | 0.0484 | 33 | 29 | 40 | DILFPYIEENVK |
| 1579.8302 | 1579.8809 | 0.0507 | 32 | 127 | 140 | MKAEFFADVVPAVR |
| 1745.8654 | 1745.9501 | 0.0847 | 49 | 112 | 126 | QLQGHMWRAAFTA |
| | | | | | | GR |
| | 1745.9501 | | | | | |
| 2878.4734 | 2878.4966 | 0.0232 | 8 | 162 | 187 | LLFGHSTEGDILELV |
| | | | | | | DG |
| | | | | | | H FDTKIGHK |
| 3854.9124 | 3855.2363 | 0.3239 | 84 | 149 | 183 | VYIYSSGSVEAQKLL |
| | | | | | | FGH |
| | | | | | | STEGDILELVDGHFD |
| | | | | | | TK |

Peptide Information

Tax_Id = 9606 Gene_Symbol = NCOR1 Nuclear
receptor co-repressor isoform 1

| | Obsrv. | | | Start | End | |
|---|---|---|---|---|---|---|
| Calc. Mass | Mass | ±da | ±ppm | Seq. | Seq. | Sequence |
| 1257.6184 | 1257.6606 | 0.0422 | 34 | 22 | 32 | SVAYMPYAEVK |
| 1257.6184 | 1257.6606 | 0.0422 | 34 | 22 | 32 | SVAYMPYAEVK |
| 1413.7195 | 1413.8362 | 0.1167 | 83 | 22 | 33 | SVAYMPYAEVKR |
| 1465.67 | 1465.804 | 0.134 | 91 | 3 | 16 | SSTSPCGTSKSPNR |
| 1465.67 | 1465.804 | 0.134 | 91 | 3 | 16 | SSTSPCGTSKSPNR |
| 1741.8398 | 1741.8694 | 0.0296 | 17 | 33 | 47 | RALEQEAQMHNTAAR |
| 2199.1809 | 2199.1213 | −0.0596 | −27 | 73 | 92 | YSVPPVLQPAPHQVIT |
| | | | | | | NL |
| | | | | | | PE |

Peptide Information

| | Obsrv. | | | Start | End | |
|---|---|---|---|---|---|---|
| Calc. Mass | Mass | ±da | ±ppm | Seq. | Seq. | Sequence |
| 842.4519 | 842.4806 | 0.0287 | 34 | 4465 | 4471 | WHVVSSK |
| 870.5229 | 870.511 | −0.0119 | −14 | 3736 | 3743 | LMALGPIR |
| 910.4265 | 910.4239 | −0.0026 | −3 | 3476 | 3482 | YSEIQDR |
| 910.4265 | 910.4239 | −0.0026 | −3 | 3476 | 3482 | YSEIQDR |
| 1021.5499 | 1021.5206 | −0.0293 | −29 | 2551 | 2558 | QQVQFMLK |
| 1021.5499 | 1021.5206 | −0.0293 | −29 | 2551 | 2558 | QQVQFMLK |
| 1170.6841 | 1170.6456 | −0.0385 | −33 | 3312 | 3321 | VVKAQIQEQK |
| 1187.6201 | 1187.673 | 0.0529 | 45 | 2757 | 2766 | NCPISAKLER |
| 1232.6117 | 1232.6179 | 0.0062 | 5 | 2659 | 2668 | QQLEETSEIR |
| 1257.6797 | 1257.6606 | −0.0191 | −15 | 1506 | 1516 | QISEQLNALNK |
| 1257.6797 | 1257.6606 | −0.0191 | −15 | 1506 | 1516 | QISEQLNALNK |
| 1261.694 | 1261.6659 | −0.0281 | −22 | 380 | 389 | LLEVWIEFGR |
| | 1261.6659 | | | | | |
| 1320.7271 | 1320.6123 | −0.1148 | −87 | 1870 | 1881 | GDLRFVTISGQK |
| 1406.7386 | 1406.7148 | −0.0238 | −17 | 4647 | 4658 | QPVYDTTIRTGR |
| 1406.7386 | 1406.7148 | −0.0238 | −17 | 4647 | 4658 | QPVYDTTIRTGR |
| 1413.7809 | 1413.8362 | 0.0553 | 39 | 3156 | 3167 | ARQEQLELTLGR |
| 1420.7213 | 1420.736 | 0.0147 | 10 | 2940 | 2951 | TGSLEEMTQRLR |
| 1425.7156 | 1425.8483 | 0.1327 | 93 | 869 | 880 | NTISVKAVCDYR |
| 1425.7156 | 1425.8483 | 0.1327 | 93 | 869 | 880 | NTISVKAVCDYR |
| 1428.7693 | 1428.7908 | 0.0215 | 15 | 5052 | 5063 | LNDALDRLEELK |
| 1450.6996 | 1450.7076 | 0.008 | 6 | 2135 | 2145 | FEQLCLQQQEK |
| 1465.7281 | 1465.804 | 0.0759 | 52 | 4428 | 4439 | EETYNQLLDKGR |
| 1465.7316 | 1465.804 | 0.0724 | 49 | 4440 | 4453 | LMLLSRDDSGSG |
| | | | | | | SK |
| 1487.7952 | 1487.7893 | −0.0059 | −4 | 3565 | 3577 | QTTGEEVLLIQEK |
| 1502.873 | 1502.896 | 0.023 | 15 | 380 | 391 | LLEVWIEFGRIK |
| 1502.873 | 1502.896 | 0.023 | 15 | 380 | 391 | LLEVWIEFGRIK |
| 1532.6785 | 1532.8113 | 0.1328 | 87 | 3891 | 3903 | ELNPEEGEMVEEK |
| 1546.8727 | 1546.7799 | −0.0928 | −60 | 4038 | 4050 | EIKFLDVLELAEK |
| 1707.7603 | 1707.8604 | 0.1001 | 59 | 889 | 902 | NDECVLEDNSQR |
| | | | | | | TK |
| 1708.8389 | 1708.9053 | 0.0664 | 39 | 3681 | 3695 | EGLDKLVSDANEQYK |
| 1713.8728 | 1713.9238 | 0.051 | 30 | 3123 | 3137 | HMLEEEGTLDLLGLK |
| 1727.9149 | 1727.9309 | 0.016 | 9 | 2151 | 2165 | KLLPQAEMFEHLSGK |
| 1813.8942 | 1813.937 | 0.0428 | 24 | 4020 | 4033 | LDQMVFFWEDIKAR |
| 1950.9412 | 1951.0114 | 0.0702 | 36 | 4960 | 4975 | ALIAEHQTFMEEMT |
| | | | | | | RK |
| 1966.9362 | 1967.0013 | 0.0651 | 33 | 4960 | 4975 | ALIAEHQTFMEEMT |
| | | | | | | RK |
| 2091.9805 | 2091.998 | 0.0175 | 8 | 496 | 511 | DENYYQLEELAFRV |
| | | | | | | MR |
| 2186.155 | 2185.9929 | −0.1621 | −74 | 1958 | 1978 | LLSDTVASDPGVLQE |
| | | | | | | QLA |
| | | | | | | TTK |
| 2186.1851 | 2186.1921 | 0.007 | 3 | 2864 | 2882 | MSELRVTLDPVQLES |
| | | | | | | SL |
| | | | | | | LR |
| 2211.1301 | 2211.2874 | 0.1573 | 71 | 5207 | 5226 | STVMVRVGGGWMA |
| | | | | | | LDE |
| | | | | | | FLVK |
| 2501.2268 | 2501.4001 | 0.1733 | 69 | 1339 | 1358 | FSQQYSTIVKDYELQ |
| | | | | | | LMT |
| | | | | | | YK |

Peptide Information

Tax_Id = 9606 Gene_Symbol = DECR2 5 kDa
protein

| | Obsrv. | | | Start | End | |
|---|---|---|---|---|---|---|
| Calc. Mass | Mass | ±da | ±ppm | Seq. | Seq. | Sequence |
| 1170.6089 | 1170.6456 | 0.0367 | 31 | 19 | 27 | HLFCPDLLR |
| 1413.7308 | 1413.8362 | 0.1054 | 75 | 19 | 29 | HLFCPDLLRDK |
| 2199.1743 | 2199.1213 | −0.053 | −24 | 30 | 50 | VAFITGGGSGIGFRIAE |
| | | | | | | MR |

Peptide Information

Tax_Id = 9606 Gene_Symbol = ENOPH1 Isoform
1 of Enolase-phosphatase E1

| | Obsrv. | | | Start | End | |
|---|---|---|---|---|---|---|
| Calc. Mass | Mass | ±da | ±ppm | Seq. | Seq. | Sequence |
| 1320.6947 | 1320.6123 | −0.0824 | −62 | 129 | 140 | AEFFADVVPAVR |
| 1479.7729 | 1479.8213 | 0.0484 | 33 | 29 | 40 | DILFPYIEENVK |
| 1579.8302 | 1579.8809 | 0.0507 | 32 | 127 | 140 | MKAEFFADVVPAVR |
| 1745.8654 | 1745.9501 | 0.0847 | 49 | 112 | 126 | QLQGHMWRAAFTAGR |
| 2878.4734 | 2878.4966 | 0.0232 | 8 | 162 | 187 | LLFGHSTEGDILELVDGH |
| | | | | | | FDTKIGHK |
| 3854.9124 | 3855.2363 | 0.3239 | 84 | 149 | 183 | VYIYSSGSVEAQKLLFGH |
| | | | | | | STEGDILELVDGHFDTK |

Peptide Information

Tax_Id = 9606 Gene_Symbol = WDR7 Isoform 2
of WD repeat-containing protein 7

| | | | | Start | End | |
|---|---|---|---|---|---|---|
| Calc. Mass | Obsrv. Mass | ±da | ±ppm | Seq. | Seq. | Sequence |
| 985.5676 | 985.5631 | −0.0045 | −5 | 868 | 877 | KLPASEGVGK |
| 985.5676 | 985.5631 | −0.0045 | −5 | 868 | 877 | KLPASEGVGK |
| 1097.5773 | 1097.5127 | −0.0646 | −59 | 1442 | 1451 | NVILMAHDGK |
| 1257.5609 | 1257.6606 | 0.0997 | 79 | 1323 | 1331 | FYMVSYYER |
| 1257.5609 | 1257.6606 | 0.0997 | 79 | 1323 | 1331 | FYMVSYYER |
| 1261.6107 | 1261.6659 | 0.0552 | 44 | 983 | 991 | WQDRCLEVR |
| 1271.6743 | 1271.6776 | 0.0033 | 3 | 1362 | 1374 | GPITAVAFAPDGR |
| 1320.7205 | 1320.6123 | −0.1082 | −82 | 636 | 647 | SLAALKNMAHHK |
| 1350.6294 | 1350.7144 | 0.085 | 63 | 1312 | 1322 | GLQECFPAICR |
| 1406.7097 | 1406.7148 | 0.0051 | 4 | 669 | 680 | YSHNSLMVQAIK |
| 1406.7097 | 1406.7148 | 0.0051 | 4 | 669 | 680 | YSHNSLMVQAIK |
| 1420.689 | 1420.736 | 0.047 | 33 | 285 | 297 | LPASCLPASDSFR |
| 1713.8846 | 1713.9238 | 0.0392 | 23 | 271 | 284 | VIIWTENGQSYIYK |
| 1951.0834 | 1951.0114 | −0.072 | −37 | 1141 | 1157 | HTCKALTFLLLQPPSPK |
| 2185.9666 | 2185.9929 | 0.0263 | 12 | 756 | 772 | EHLLDDEEEDEEIMRQR |
| 3038.4968 | 3038.5745 | 0.0777 | 26 | 480 | 505 | YDQRYLISGGVDFSVIIW |
| | | | | | | DIFSGEMK |
| 3854.9578 | 3855.2363 | 0.2785 | 72 | 949 | 981 | QGWSQLAAMHCVMLP D |
| | | | | | | LLGLDKFRPPLLEMLAR |

Peptide Information

Tax_Id = 9606 Gene_Symbol = WDR7 Isoform 2 of WD
repeat-containing protein 7

| | Obsrv. | | | Start | End | |
|---|---|---|---|---|---|---|
| Calc. Mass | Mass | ±da | ±ppm | Seq. | Seq. | Sequence |
| 985.5676 | 985.5671 | −0.0005 | −1 | 868 | 877 | KLPASEGVGK |
| 1257.5609 | 1257.6626 | 0.1017 | 81 | 1323 | 1331 | FYMVSYYER |
| 1261.6107 | 1261.6564 | 0.0457 | 36 | 983 | 991 | WQDRCLEVR |
| 1261.6107 | 1261.6564 | 0.0457 | 36 | 983 | 991 | WQDRCLEVR |
| 1271.6743 | 1271.6829 | 0.0086 | 7 | 1362 | 1374 | GPITAVAFAPDGR |
| 1320.7205 | 1320.6122 | −0.1083 | −82 | 636 | 647 | SLAALKNMAHHK |
| 1320.7205 | 1320.6122 | −0.1083 | −82 | 636 | 647 | SLAALKNMAHHK |
| 1350.6294 | 1350.6978 | 0.0684 | 51 | 1312 | 1322 | GLQECFPAICR |
| 1406.7097 | 1406.7087 | −0.001 | −1 | 669 | 680 | YSHNSLMVQAIK |
| 1406.7097 | 1406.7087 | −0.001 | −1 | 669 | 680 | YSHNSLMVQAIK |
| 1420.689 | 1420.7378 | 0.0488 | 34 | 285 | 297 | LPASCLPASDSFR |
| 1713.8846 | 1713.9213 | 0.0367 | 21 | 271 | 284 | VIIWTENGQSYIYK |
| 1901.8069 | 1901.9828 | 0.1759 | 92 | 756 | 770 | EHLLDDEEEDEEIMR |
| 1951.0834 | 1950.9768 | −0.1066 | −55 | 1141 | 1157 | HTCKALTFLLLQPPSPK |
| 2092.2278 | 2092.0271 | −0.2007 | −96 | 1224 | 1243 | HALSLIATARPPAFIT |
| | | | | | | TIAK |
| 2185.9666 | 2186.0493 | 0.0827 | 38 | 756 | 772 | EHLLDDEEEDEEIMR |
| | | | | | | QR |
| 2185.9666 | 2186.0493 | 0.0827 | 38 | 756 | 772 | EHLLDDEEEDEEIMR |
| | | | | | | QR |
| 2233.1296 | 2233.1709 | 0.0413 | 18 | 1354 | 1374 | CQTIHGHKGPITAVA |
| | | | | | | FAP |
| | | | | | | DGR |
| 3038.4968 | 3038.5193 | 0.0225 | 7 | 480 | 505 | YDQRYLISGGVDFSV |
| | | | | | | IIW |
| | | | | | | DIFSGEMK |

Peptide Information

Tax_Id = 9606 Gene_Symbol = WDR7 Isoform 1
of WD repeat-containing protein 7

| | Obsrv. | | | Start | End | |
|---|---|---|---|---|---|---|
| Calc. Mass | Mass | ±da | ±ppm | Seq. | Seq. | Sequence |
| 985.5676 | 985.5671 | −0.0005 | −1 | 868 | 877 | KLPASEGVGK |
| 1257.5609 | 1257.6626 | 0.1017 | 81 | 1356 | 1364 | FYMVSYYER |
| 1261.6107 | 1261.6564 | 0.0457 | 36 | 1016 | 1024 | WQDRCLEVR |
| 1261.6107 | 1261.6564 | 0.0457 | 36 | 1016 | 1024 | WQDRCLEVR |
| 1271.6743 | 1271.6829 | 0.0086 | 7 | 1395 | 1407 | GPITAVAFAPDGR |
| 1320.7205 | 1320.6122 | −0.1083 | −82 | 636 | 647 | SLAALKNMAHHK |
| 1320.7205 | 1320.6122 | −0.1083 | −82 | 636 | 647 | SLAALKNMAHHK |
| 1350.6294 | 1350.6978 | 0.0684 | 51 | 1345 | 1355 | GLQECFPAICR |
| 1406.7097 | 1406.7087 | −0.001 | −1 | 669 | 680 | YSHNSLMVQAIK |
| 1406.7097 | 1406.7087 | −0.001 | −1 | 669 | 680 | YSHNSLMVQAIK |
| 1420.689 | 1420.7378 | 0.0488 | 34 | 285 | 297 | LPASCLPASDSFR |
| 1713.8846 | 1713.9213 | 0.0367 | 21 | 271 | 284 | VIIWTENGQSYIYK |
| 1901.8069 | 1901.9828 | 0.1759 | 92 | 756 | 770 | EHLLDDEEEDEEIMR |
| 1951.0834 | 1950.9768 | −0.1066 | −55 | 1174 | 1190 | HTCKALTFLLLQPPSPK |
| 2092.2278 | 2092.0271 | −0.2007 | −96 | 1257 | 1276 | HALSLIATARPPAFITTIA |
| 2185.9666 | 2186.0493 | 0.0827 | 38 | 756 | 772 | EHLLDDEEEDEEIMRQR |
| 2185.9666 | 2186.0493 | 0.0827 | 38 | 756 | 772 | EHLLDDEEEDEEIMRQR |
| 2233.1296 | 2233.1709 | 0.0413 | 18 | 1387 | 1407 | CQTIHGHKGPITAVAFAPDGR |
| 3038.4968 | 3038.5193 | 0.0225 | 7 | 480 | 505 | YDQRYLISGGVDFSVIIW |
| IP101008928 | | | | | | DIFSGEMK |

Peptide Information

Tax_Id = 9606 Gene_Symbol = -Myosin-reactive
immunoglobulin heavy chain variable region
(Fragment)

| | Obsrv. | | | Start | End | |
|---|---|---|---|---|---|---|
| Calc. Mass | Mass | ±da | ±ppm | Seq. | Seq. | Sequence |
| 1287.6791 | 1287.6769 | −0.0022 | −2 | 1 | 12 | EVQLVESGAEVK |
| 1320.5525 | 1320.6122 | 0.0597 | 45 | 88 | 98 | SDDTAVYYCAR |
| 1320.5525 | 1320.6122 | 0.0597 | 45 | 88 | 98 | SDDTAVYYCAR |
| 1838.8319 | 1839.0062 | 0.1743 | 95 | 24 | 38 | ASGYTFTGYYMHWVR |
| 2092.0049 | 2092.0271 | 0.0222 | 11 | 68 | 85 | VTMTRDTTISTAYMEL |
| | | | | | | SR |
| 2096.9958 | 2097.0576 | 0.0618 | 29 | 105 | 125 | IAAAGDAFDIWGQGT |
| | | | | | | MVT |
| | | | | | | VSS |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5264 | 0.0035 | 4 | 3736 | 3743 | LMALGPIR |
| 870.5229 | 870.5264 | 0.0035 | 4 | 3736 | 3743 | LMALGPIR |
| 910.4265 | 910.4365 | 0.01 | 11 | 3476 | 3482 | YSEIQDR |
| 1021.5499 | 1021.5289 | −0.021 | −21 | 2551 | 2558 | QQVQFMLK |
| 1021.5499 | 1021.5289 | −0.021 | −21 | 2551 | 2558 | QQVQFMLK |
| 1170.6841 | 1170.6501 | −0.034 | −29 | 3312 | 3321 | VVKAQIQEQK |
| 1187.6201 | 1187.6735 | 0.0534 | 45 | 2757 | 2766 | NCPISAKLER |
| 1187.6201 | 1187.6735 | 0.0534 | 45 | 2757 | 2766 | NCPISAKLER |
| 1225.6497 | 1225.5806 | −0.0691 | −56 | 3958 | 3968 | MPPLIPAEVDK |
| 1232.6117 | 1232.6139 | 0.0022 | 2 | 2659 | 2668 | QQLEETSEIR |
| 1257.6797 | 1257.6626 | −0.0171 | −14 | 1506 | 1516 | QISEQLNALNK |
| 1261.694 | 1261.6564 | −0.0376 | −30 | 380 | 389 | LLEVWIEFGR |
| 1261.694 | 1261.6564 | −0.0376 | −30 | 380 | 389 | LLEVWIEFGR |
| 1287.6791 | 1287.6769 | −0.0022 | −2 | 4662 | 4672 | EKTLLPEDSQK |
| 1320.7271 | 1320.6122 | −0.1149 | −87 | 1870 | 1881 | GDLRFVTISGQK |
| 1320.7271 | 1320.6122 | −0.1149 | −87 | 1870 | 1881 | GDLRFVTISGQK |
| 1406.7386 | 1406.7087 | −0.0299 | −21 | 4647 | 4658 | QPVYDTTIRTGR |
| 1406.7386 | 1406.7087 | −0.0299 | −21 | 4647 | 4658 | QPVYDTTIRTGR |
| 1413.7809 | 1413.825 | 0.0441 | 31 | 3156 | 3167 | ARQEQLELTLGR |
| 1420.7213 | 1420.7378 | 0.0165 | 12 | 2940 | 2951 | TGSLEEMTQRLR |
| 1425.7156 | 1425.8256 | 0.11 | 77 | 869 | 880 | NTISVKAVCDYR |
| 1450.6996 | 1450.6963 | −0.0033 | −2 | 2135 | 2145 | FEQLCLQQQEK |
| 1465.7281 | 1465.7937 | 0.0656 | 45 | 4428 | 4439 | EETYNQLLDKGR |
| 1465.7316 | 1465.7937 | 0.0621 | 42 | 4440 | 4453 | LMLLSRDDSGSGSK |
| 1502.873 | 1502.8854 | 0.0124 | 8 | 380 | 391 | LLEVWIEFGRIK |
| 1532.6785 | 1532.8059 | 0.1274 | 83 | 3891 | 3903 | ELNPEEGEMVEEK |
| 1546.8727 | 1546.7936 | −0.0791 | −51 | 4038 | 4050 | EIKFLDVLELAEK |
| 1713.8728 | 1713.9213 | 0.0485 | 28 | 3123 | 3137 | HMLEEEGTLDLLG |
| | | | | | | LK |
| 1794.9636 | 1794.8539 | −0.1097 | −61 | 5106 | 5121 | QEFIDGILASKFPT |
| | | | | | | TK |
| 1838.8412 | 1839.0062 | 0.165 | 90 | 4960 | 4974 | ALIAEHQTFMEEMTR |
| 1950.9412 | 1950.9768 | 0.0356 | 18 | 4960 | 4975 | ALIAEHQTFMEEMTRK |
| 1966.9362 | 1966.9713 | 0.0351 | 18 | 4960 | 4975 | ALIAEHQTFMEEMTRK |
| 2092.0266 | 2092.0271 | 0.0005 | 0 | 2275 | 2293 | WLKETEGSIPPTETSM |
| | | | | | | SAK |
| 2186.155 | 2186.0493 | −0.1057 | −48 | 1958 | 1978 | LLSDTVASDPGVLQE |
| | | | | | | QLA |
| | | | | | | TTK |
| 2186.155 | 2186.0493 | −0.1057 | −48 | 1958 | 1978 | LLSDTVASDPGVLQE |
| | | | | | | QLA |
| | | | | | | TTK |
| 2200.0632 | 2200.0908 | 0.0276 | 13 | 4015 | 4031 | EIQDKLDQMVFFWED |
| | | | | | | IK |
| 2233.1135 | 2233.1709 | 0.0574 | 26 | 2462 | 2481 | EALAGLLVTYPNSQE |
| | | | | | | AEN |
| | | | | | | WK |
| 2299.0217 | 2299.2339 | 0.2122 | 92 | 3068 | 3088 | EMFSQLADLDDELDG |
| | | | | | | MG |
| | | | | | | AIGR |
| 2501.2268 | 2501.3357 | 0.1089 | 44 | 1339 | 1358 | FSQQYSTIVKDYELQL |
| | | | | | | MT |
| | | | | | | YK |

Peptide Information

Tax_Id = 9606 Gene_Symbol = NASP
Uncharacterized protein

| | Obsrv. | | | Start | End | |
|---|---|---|---|---|---|---|
| Calc. Mass | Mass | ±da | ±ppm | Seq. | Seq. | Sequence |
| 912.4322 | 912.4548 | 0.0226 | 25 | 40 | 46 | WADHEVR |
| 1851.9004 | 1852.002 | 0.1023 | 55 | 2 | 20 | AMESTATAAVAAELV |
| | | | | | | SADK |
| 1966.946 | 1966.9713 | 0.0253 | 13 | 1 | 20 | MAMESTATAAVAAEL |
| | | | | | | VS |
| | | | | | | ADK |
| 2299.0906 | 2299.2339 | 0.1433 | 62 | 2 | 24 | AMESTATAAVAAELV |
| | | | | | | SAD |
| | | | | | | KMSGR |

Peptide Information

Tax_Id = 9606 Gene_Symbol = MACF1 Isoform
3 of Microtubule-actin cross-linking factor 1,
isoforms 1/2/3/5

| | Obsrv. | | | Start | End | |
|---|---|---|---|---|---|---|
| Calc. Mass | Mass | ±da | ±ppm | Seq. | Seq. | Sequence |
| 870.5229 | 870.5264 | 0.0035 | 4 | 3680 | 3687 | LMALGPIR |
| 870.5229 | 870.5264 | 0.0035 | 4 | 3680 | 3687 | LMALGPIR |
| 910.4265 | 910.4365 | 0.01 | 11 | 3420 | 3426 | YSEIQDR |
| 1021.5499 | 1021.5289 | −0.021 | −21 | 2495 | 2502 | QQVQFMLK |
| 1021.5499 | 1021.5289 | −0.021 | −21 | 2495 | 2502 | QQVQFMLK |
| 1170.6841 | 1170.6501 | −0.034 | −29 | 3256 | 3265 | VVKAQIQEQK |
| 1187.6201 | 1187.6735 | 0.0534 | 45 | 2701 | 2710 | NCPISAKLER |
| 1187.6201 | 1187.6735 | 0.0534 | 45 | 2701 | 2710 | NCPISAKLER |
| 1225.6497 | 1225.5806 | −0.0691 | −56 | 3902 | 3912 | MPPLIPAEVDK |
| | 1225.5806 | | | | | |
| | 9 | | | | | |
| 1257.6797 | 1257.6626 | −0.0171 | −14 | 1471 | 1481 | QISEQLNALNK |
| 1261.694 | 1261.6564 | −0.0376 | −30 | 345 | 354 | LLEVWIEFGR |
| 1261.694 | 1261.6564 | −0.0376 | −30 | 345 | 354 | LLEVWIEFGR |
| 1287.6791 | 1287.6769 | −0.0022 | −2 | 4606 | 4616 | EKTLLPEDSQK |
| 1320.7271 | 1320.6122 | −0.1149 | −87 | 1835 | 1846 | GDLRFVTISGQK |
| 1320.7271 | 1320.6122 | −0.1149 | −87 | 1835 | 1846 | GDLRFVTISGQK |
| 1406.7386 | 1406.7087 | −0.0299 | −21 | 4591 | 4602 | QPVYDTTIRTGR |
| 1406.7386 | 1406.7087 | −0.0299 | −21 | 4591 | 4602 | QPVYDTTIRTGR |
| 1413.7809 | 1413.825 | 0.0441 | 31 | 3100 | 3111 | ARQEQLELTLGR |
| 1420.7213 | 1420.7378 | 0.0165 | 12 | 2884 | 2895 | TGSLEEMTQRLR |
| 1425.7156 | 1425.8256 | 0.11 | 77 | 834 | 845 | NTISVKAVCDYR |
| 1450.6996 | 1450.6963 | −0.0033 | −2 | 2100 | 2110 | FEQLCLQQQEK |
| 1465.7281 | 1465.7937 | 0.0656 | 45 | 4372 | 4383 | EETYNQLLDKGR |
| 1465.7316 | 1465.7937 | 0.0621 | 42 | 4384 | 4397 | LMLLSRDDSGSGSK |
| 1502.873 | 1502.8854 | 0.0124 | 8 | 345 | 356 | LLEVWIEFGRIK |
| 1532.6785 | 1532.8059 | 0.1274 | 83 | 3835 | 3847 | ELNPEEGEMVEEK |
| 1546.8727 | 1546.7936 | −0.0791 | −51 | 3982 | 3994 | EIKFLDVLELAEK |
| 1713.8728 | 1713.9213 | 0.0485 | 28 | 3067 | 3081 | HMLEEEGTLDLLGLK |
| 1794.9636 | 1794.8539 | −0.1097 | −61 | 5050 | 5065 | QEFIDGILASKFPTTK |
| 1838.8412 | 1839.0062 | 0.165 | 90 | 4904 | 4918 | ALIAEHQTFMEEMTR |
| 1950.9412 | 1950.9768 | 0.0356 | 18 | 4904 | 4919 | ALIAEHQTFMEEMTRK |
| 1966.9362 | 1966.9713 | 0.0351 | 18 | 4904 | 4919 | ALIAEHQTFMEEMTRK |
| 2092.0266 | 2092.0271 | 0.0005 | 0 | 2240 | 2258 | WLKETEGSIPPTETSM |
| | | | | | | SAK |
| 2186.155 | 2186.0493 | −0.1057 | −48 | 1923 | 1943 | LLSDTVASDPGVLQE |
| | | | | | | QLA |
| | | | | | | TTK |
| 2186.155 | 2186.0493 | −0.1057 | −48 | 1923 | 1943 | LLSDTVASDPGVLQE |
| | | | | | | QLA |
| | | | | | | TTK |
| 2200.0632 | 2200.0908 | 0.0276 | 13 | 3959 | 3975 | EIQDKLDQMVFFWED |
| | | | | | | IK |
| 2233.1135 | 2233.1709 | 0.0574 | 26 | 2406 | 2425 | EALAGLLVTYPNSQE |
| | | | | | | AEN |
| | | | | | | WK |
| 2299.0217 | 2299.2339 | 0.2122 | 92 | 3012 | 3032 | EMFSQLADLDDELDG |
| | | | | | | MG |
| | | | | | | AIGR |
| 2501.2268 | 2501.3357 | 0.1089 | 44 | 1304 | 1323 | FSQQYSTIVKDYELQL |
| | | | | | | MT |
| | | | | | | YK |

Peptide Information

| | Obsrv. | | | Start | End | |
|---|---|---|---|---|---|---|
| Calc. Mass | Mass | ±da | ±ppm | Seq. | Seq. | Sequence |
| 870.5229 | 870.5264 | 0.0035 | 4 | 3606 | 3613 | LMALGPIR |
| 870.5229 | 870.5264 | 0.0035 | 4 | 3606 | 3613 | LMALGPIR |
| 1021.5499 | 1021.5289 | −0.021 | −21 | 2530 | 2537 | QQVQFMLK |
| 1021.5499 | 1021.5289 | −0.021 | −21 | 2530 | 2537 | QQVQFMLK |
| 1170.6841 | 1170.6501 | −0.034 | −29 | 3291 | 3300 | VVKAQIQEQK |
| 1187.6201 | 1187.6735 | 0.0534 | 45 | 2736 | 2745 | NCPISAKLER |
| 1187.6201 | 1187.6735 | 0.0534 | 45 | 2736 | 2745 | NCPISAKLER |
| 1225.6497 | 1225.5806 | −0.0691 | −56 | 3828 | 3838 | MPPLIPAEVDK |
| 1232.6117 | 1232.6139 | 0.0022 | 2 | 2638 | 2647 | QQLEETSEIR |
| 1257.6797 | 1257.6626 | −0.0171 | −14 | 1506 | 1516 | QISEQLNALNK |
| 1261.694 | 1261.6564 | −0.0376 | −30 | 380 | 389 | LLEVWIEFGR |
| 1261.694 | 1261.6564 | −0.0376 | −30 | 380 | 389 | LLEVWIEFGR |
| 1287.6791 | 1287.6769 | −0.0022 | −2 | 4532 | 4542 | EKTLLPEDSQK |
| 1320.7271 | 1320.6122 | −0.1149 | −87 | 1870 | 1881 | GDLRFVTISGQK |
| 1320.7271 | 1320.6122 | −0.1149 | −87 | 1870 | 1881 | GDLRFVTISGQK |
| 1406.7386 | 1406.7087 | −0.0299 | −21 | 4517 | 4528 | QPVYDTTIRTGR |
| 1406.7386 | 1406.7087 | −0.0299 | −21 | 4517 | 4528 | QPVYDTTIRTGR |
| 1413.7809 | 1413.825 | 0.0441 | 31 | 3135 | 3146 | ARQEQLELTLGR |
| 1420.7213 | 1420.7378 | 0.0165 | 12 | 2919 | 2930 | TGSLEEMTQRLR |
| 1425.7156 | 1425.8256 | 0.11 | 77 | 869 | 880 | NTISVKAVCDYR |
| 1450.6996 | 1450.6963 | −0.0033 | −2 | 2135 | 2145 | FEQLCLQQQEK |
| 1465.7281 | 1465.7937 | 0.0656 | 45 | 4298 | 4309 | EETYNQLLDKGR |
| 1465.7316 | 1465.7937 | 0.0621 | 42 | 4310 | 4323 | LMLLSRDDSGSGSK |
| 1502.873 | 1502.8854 | 0.0124 | 8 | 380 | 391 | LLEVWIEFGRIK |
| 1532.6785 | 1532.8059 | 0.1274 | 83 | 3761 | 3773 | ELNPEEGEMVEEK |
| 1546.8727 | 1546.7936 | −0.0791 | −51 | 3908 | 3920 | EIKFLDVLELAEK |
| 1713.8728 | 1713.9213 | 0.0485 | 28 | 3102 | 3116 | HMLEEEGTLDLLGLK |
| 1794.9636 | 1794.8539 | −0.1097 | −61 | 4976 | 4991 | QEFIDGILASKFPTTK |
| 1838.8412 | 1839.0062 | 0.165 | 90 | 4830 | 4844 | ALIAEHQTFMEEMTR |
| 1950.9412 | 1950.9768 | 0.0356 | 18 | 4830 | 4845 | ALIAEHQTFMEEMT |
| | | | | | | RK |
| 1966.9362 | 1966.9713 | 0.0351 | 18 | 4830 | 4845 | ALIAEHQTFMEEMT |
| | | | | | | RK |
| 2092.0266 | 2092.0271 | 0.0005 | 0 | 2275 | 2293 | WLKETEGSIPPTETSM |
| | | | | | | SAK |
| 2186.155 | 2186.0493 | −0.1057 | −48 | 1958 | 1978 | LLSDTVASDPGVLQE |
| | | | | | | QLA |
| | | | | | | TTK |
| 2186.155 | 2186.0493 | −0.1057 | −48 | 1958 | 1978 | LLSDTVASDPGVLQE |
| | | | | | | QLA |
| | | | | | | TTK |
| 2200.0632 | 2200.0908 | 0.0276 | 13 | 3885 | 3901 | EIQDKLDQMVFFWED |
| | | | | | | IK |
| 2233.1135 | 2233.1709 | 0.0574 | 26 | 2441 | 2460 | EALAGLLVTYPNSQE |
| | | | | | | AEN |
| | | | | | | WK |
| 2299.0217 | 2299.2339 | 0.2122 | 92 | 3047 | 3067 | EMFSQLADLDDELDG |
| | | | | | | MG |
| | | | | | | AIGR |
| 2501.2268 | 2501.3357 | 0.1089 | 44 | 1339 | 1358 | FSQQYSTIVKDYELQL |
| | | | | | | MT |
| | | | | | | YK |

Peptide Information

Tax_Id = 9606 Gene_Symbol = HSD17B12
Estradiol 17-beta-dehydrogenas e 12

| | | | | Start | End | |
|---|---|---|---|---|---|---|
| Calc. Mass | Obsrv. Mass | ±da | ±ppm | Seq. | Seq. | Sequence |
| 910.4516 | 910.4365 | −0.0151 | −17 | 65 | 72 | SYAEELAK |
| 1170.6517 | 1170.6501 | −0.0016 | −1 | 26 | 35 | ISYSLFTALR |
| 1225.6028 | 1225.5806 | −0.0222 | −18 | 293 | 302 | IVMNMNKSTR |
| 1261.6635 | 1261.6564 | −0.0071 | −6 | 85 | 95 | DKLDQVSSEIK |
| 1261.6635 | 1261.6564 | −0.0071 | −6 | 85 | 95 | DKLDQVSSEIK |
| 1320.7014 | 1320.6122 | −0.0892 | −68 | 157 | 167 | MININILSVCK |
| 1320.7014 | 1320.6122 | −0.0892 | −68 | 157 | 167 | MININILSVCK |
| 1967.1365 | 1966.9713 | −0.1652 | −84 | 224 | 241 | GVFVQSVLPYFVATKLAK |
| 2691.4065 | 2691.3652 | −0.0413 | −15 | 157 | 179 | MININILSVCKMTQLV |
| | | | | | | LPG |
| | | | | | | MVER |
| 2707.4014 | 2707.4404 | 0.039 | 14 | 157 | 179 | MININILSVCKMTQLV |
| | | | | | | LPG |
| | | | | | | MVER |

Peptide Information

Tax_Id = 9606 Gene_Symbol = KRT2 Keratin,
type II cytoskeletal 2 epidermal

| | | | | Start | End | |
|---|---|---|---|---|---|---|
| Calc. Mass | Obsrv. Mass | ±da | ±ppm | Seq. | Seq. | Sequence |
| 910.4152 | 910.4365 | 0.0213 | 23 | 274 | 280 | YEDEINK |
| 985.5789 | 985.5671 | −0.0118 | −12 | 460 | 467 | EDLARLLR |
| 1254.6074 | 1254.6842 | 0.0768 | 61 | 21 | 34 | GFSSGSAVVSGGSR |
| 1287.6111 | 1287.6769 | 0.0658 | 51 | 35 | 45 | RSTSSFSCLSR |
| 1320.5829 | 1320.6122 | 0.0293 | 22 | 46 | 61 | HGGGGGGFGGGGF |
| | | | | | | GSR |
| 1320.5829 | 1320.6122 | 0.0293 | 22 | 46 | 61 | HGGGGGGFGGGGF |
| | | | | | | GSR |
| 1740.7057 | 1740.7649 | 0.0592 | 34 | 531 | 550 | GSSSGGGYSSGSSSY |
| | | | | | | GS |
| | | | | | | GGR |
| 1745.8235 | 1745.9114 | 0.0879 | 50 | 422 | 436 | QCKNVQDAIADAEQR |
| 1838.9144 | 1839.0062 | 0.0918 | 50 | 71 | 92 | SISISVAGGGGGFGAAG |
| | | | | | | GFGGR |
| 2384.2166 | 2384.16655 | −0.0501 | −21 | 468 | 487 | DYQELMNVKLALDV |
| | | | | | | EIAT |
| | | | | | | YR |

Peptide Information

Tax_Id = 9606 Gene_Symbol = LOC731282
hypothetical protein LOC731282

| | Obsrv. | | | Start | End | |
|---|---|---|---|---|---|---|
| Calc. Mass | Mass | ±da | ±ppm | Seq. | Seq. | Sequence |
| 856.4747 | 856.5074 | 0.0327 | 38 | 297 | 304 | NPGSLRGR |
| 912.4356 | 912.4548 | 0.0192 | 21 | 170 | 176 | LETHPCR |
| 985.5425 | 985.5671 | 0.0246 | 25 | 9 | 18 | GSIGQSAIPR |
| 1350.7311 | 1350.6978 | −0.0333 | −25 | 119 | 130 | SPCPIRSPLPAR |
| 1745.8929 | 1745.9114 | 0.0185 | 11 | 82 | 98 | ASAPWASLSTRADSGLR |
| 1901.975 | 1901.9828 | 0.0078 | 4 | 1 | 18 | MSPLETNKGSIGQSAIPR |
| 2384.2722 | 2384.1665 | −0.1057 | −44 | 238 | 259 | ATSASLPQETPFALSV |
| | | | | | | VW |
| | | | | | | APRR |

8 APOA1 Apolipoprotein A-I

Apolipoprotein A-I is a protein that in humans is encoded by the APOA1 gene. It has a specific role in lipid metabolism. Apolipoprotein A-I is the major protein component of high density lipoprotein (HDL) in plasma. Chylomicrons secreted from the intestinal enterocyte also contain ApoA1 but it is quickly transferred to HDL in the bloodstream. The protein promotes cholesterol efflux from tissues to the liver for excretion. It is a cofactor for lecithin cholesterolacyltransferase (LCAT) which is responsible for the formation of most plasma cholesteryl esters. ApoA-I was also isolated as a prostacyclin (PGI2) stabilizing factor, and thus may have an anticlotting effect. Defects in the gene encoding it are associated with HDL deficiencies, including Tangier disease, and with systemic non-neuropathic amyloidosis

9 APOA1 Apolipoprotein A-I

Please see above

10 APOA1 Apolipoprotein A-I

Please refer to Nr 8

11 APOA1 Apolipoprotein A-I

Please refer to Nr 8

12 Human albumin

Human serum albumin is the most abundant protein in human blood plasma. It is produced in the liver. Albumin constitutes about half of the blood serum protein. It is soluble and monomeric. Albumin transports hormones, fatty acids, and other compounds, buffers pH, and maintains osmotic pressure, among other functions. Albumin is synthesized in the liver as preproalbumin, which has an N-terminal peptide that is removed before the nascent protein is released from the rough endoplasmic reticulum. The product, proalbumin, is in turn cleaved in the Golgi vesicles to produce the secreted albumin.

13 Transferrin

Transferrins are iron-binding blood plasma glycoproteins that control the level of free iron in biological fluids.[1] In humans, it is encoded by the TF gene.

Transferrin is a glycoprotein that binds iron very tightly but reversibly. Although iron bound to transferrin is less than

0.1% (4 mg) of the total body iron, it is the most important iron pool, with the highest rate of turnover (25 mg/24 h). Transferrin has a molecular weight of around 80 kDa and contains 2 specific high-affinity Fe(III) binding sites. The affinity of transferrin for Fe(III) is extremely high (1023 M-1 at pH 7.4) but decreases progressively with decreasing pH below neutrality. When not bound to iron, it is known as “apo-transferrin” (see also apoprotein).

14 Vimentin

Vimentin is a type III intermediate filament (IF) protein that is expressed in mesenchymal cells. IF proteins are found in all metazoan cells as well as bacteria. IF, along with tubulin-based microtubules and actin-based microfilaments, comprise the cytoskeleton. All IF proteins are expressed in a highly developmentally-regulated fashion; vimentin is the

major cytoskeletal component of mesenchymal cells. Because of this, vimentin is often used as a marker of mesenchymally-derived cells or cells undergoing an epithelial-to-mesenchymal transition (EMT) during both normal development and metastatic progression.

15 Haptoglobin

Haptoglobin (abbreviated as Hp) is a protein that in humans is encoded by the HP gene. In blood plasma, haptoglobin binds free hemoglobin (Hb) released from erythrocytes with high affinity and thereby inhibits its oxidative activity. The haptoglobin-hemoglobin complex will then be removed by the reticuloendothelial system (mostly the spleen). In clinical settings, the haptoglobulin assay is used to screen for and monitor intravascular hemolytic anemia. In intravascular hemolysis free hemoglobin will be released into circulation and hence haptoglobin will bind the Hb. This causes a decline in Hp levels. Conversely, in extravascular hemolysis the reticuloendothelial system, especially -splenic monocytes, phagocytose the erythrocytes and hemoglobin is not released into circulation and hence haptoglobin levels are normal.

Fr.IV1+IV4 ppt

Description

FIG. 239—Flow chart of AFOD01 FROM FrIV1+IV4 PASTE

PROCESS OF AFOD01 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C.

4, to go to centrifugation at temperature of 20 C, obtain the paste, called paste41.

5, to dissolve the paste with TRIS-HCL buffer (PH8.50?), dilution ratio is 1:9?, temperature is 15-20 C?

6, to perform filtration with depth filters such as 10 cp, 90 sp followed by 0.45 μm, obtain the clear filtrate.

7, to add tween80 to concentration of 1% and TNBP to 0.3%, then keep the temperature of the solution at 25 for 6 hours.

8 to cool down the solution to temperature below 10 C and adjust PH value to about ?.

9, to perform filtration with depth filters such as 10 cp, 90 sp, then followed by 0.45 μm, obtain the clear filtrate.

10, to concentrate the solution to 3%? with ultra-filtration membrane, then dialysis with 10 volume of cold WFI.

11, to carry out DV20 filtration

12, to concentrate the solution to 7.5%? protein, and adjust the PH value to 7.00.

13, to add albumin to concentration of 2.5%? as stabilizer.

14, to go to sterile filtration and filling.

FIG. 240—Flow chart of AFOD02 FROM FrIV1+IV4 PASTE

Description

PROCESS OF AFOD02 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20.

4, to go to centrifugation at temperature of 20, obtain the paste, called paste41.

5, to dissolve the paste with TRIS-HCL buffer (PH8.50?), dilution ratio is 1:9?, temperature is 15-20?

6, to perform filtration with depth filters such as 10 cp, 90 sp followed by 0.45 nm, obtain the clear filtrate.

7, to add tween80 to concentration of 1% and TNBP to 0.3%, then keep the temperature of the solution at 25 for 6 hours.

8 to cool down the solution to temperature below 10 and adjust PH value to about ?.

9, to perform filtration with depth filters such as 10 cp, 90 sp, then followed by 0.45 nm, obtain the clear filtrate.

10, to concentrate the solution to 3%? With 10 k ultra-filtration membrane, collect permeate.

11, to concentrate the permeate to 3%? With 1-3 k ultra-filtration membrane, then dialysis with 10 volume of cold WFI

12, to carry out DV20 filtration

13, to concentrate the solution to 7.5%? protein, and adjust the PH value to 7.00.

14, to add albumin to concentration of 2.5%? as stabilizer.

15, to go to sterile filtration and filling.

Description

PROCESS OF AFOD03 FROM FrIV1+IV4 PASTE

FIG. 241—Flow chart of AFOD03 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20.

4, to go to centrifugation at temperature of 20, obtain the paste, called paste41.

5, to dissolve the paste with TRIS-HCL buffer (PH8.50?), dilution ratio is 1:9?, temperature is 15-20?

6, to perform filtration with depth filters such as 10 cp, 90 sp followed by 0.45 nm, obtain the clear filtrate.

7, to add tween80 to concentration of 1% and TNBP to 0.3%, then keep the temperature of the solution at 25 for 6 hours.

8 to cool down the solution to temperature below 10 and adjust PH value to about ?.

9, to perform filtration with depth filters such as 10 cp, 90 sp, then followed by 0.45 nm, obtain the clear filtrate.

10, to concentrate the solution to 3%? With 10 k ultra-filtration membrane, collect permeate.

11, to concentrate the permeate to 3%? With 1-3 k ultra-filtration membrane, then dialysis with

10 volume of cold WFI

12, to carry out DV20 filtration

13, to concentrate the solution to 7.5%? protein, and adjust the PH value to 7.00.

14, to add albumin to concentration of 2.5%? as stabilizer.

15, to go to sterile filtration and filling.

Sterile filtration and filling

FIG. 242—Flow chart of AFOD 04 FROM FrIV1+IV4 PASTE

Description

PROCESS OF AFOD04 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20

4, to go to centrifugation at temperature of 15-20, obtain the supernatant.

5, to perform filtration with depth filters such as 10 cp, 90 sp followed by 0.45 nm, obtain the clear filtrate.

6, to add tween80 to concentration of 1% and TNBP to 0.3%, then keep the temperature of the solution at 25 for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), eluted with 90 mM NaclTRIS-HCL buffer (PH8.50). Collect elutionl.

10, to concentrate the solution to 3%? With 10 k ultra-filtration membrane, then dialysis with 10 volume of cold WFI.

11, to carry out DV20 filtration

12, to concentrate the solution to 7.5%? protein, and adjust the PH value to 7.00.

13, to add albumin to concentration of 2.5%? as stabilizer.

14, to go to sterile filtration and filling.

FIG. 243—Flow chart of AFOD 05 FROM FrIV1+IV4 PASTE

Description

PROCESS OF AFOD05 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), eluted with 60 mM Nacl TRIS-HCL buffer (PH8.50). Collect elute, called elute2.

10, to concentrate the solution to 3%? With 10 k ultra-filtration membrane, collect permeate,

11, to concentrate the permeate to 3%? With 1-3 k ultra-filtration membrane, then dialysis with 10 volume of cold WFI

12, to carry out DV20 filtration

13, to concentrate the solution to 5%? protein, and adjust the PH value to 7.00.

14, to add albumin to concentration of 2.5%? as stabilizer.

15, to go to sterile filtration and filling.

FIG. 244—Flow chart of AFOD 06 FROM FrIV1+IV4 PASTE

Description

PROCESS OF AFOD06 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), eluted with 60 mM Nacl TRIS-HCL buffer (PH8.50). Collect elute, called elute2.

10, to concentrate the solution to 7.5%? With 10 k ultra-filtration membrane, then dialysis with 10 volume of cold WFI.

11, to adjust the PH value to 6.70-7.30,

12,carry out DV20 filtration

13, to add albumin to concentration of 2.5%? as stabilizer.

14, to go to sterile filtration and filling.

FIG. 245—Flow chart of AFOD 07 FROM FrIV1+IV4 PASTE

Description

PROCESS OF AFOD07 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), eluted with 2M Nacl TRIS-HCL buffer (PH8.50). Collect elute, called elute3.

10, to concentrate the solution to 5%? With 10 k ultra-filtration membrane, collect permeate,

11, to concentrate the permeate to 3%? With 1-3 k ultra-filtration membrane, then dialysis with

10 volume of cold WFI

12, to carry out DV20 filtration

13, and adjust the PH value to 7.00.

14, to add albumin to concentration of 2.5%? as stabilizer.

15, to go to sterile filtration and filling.

FIG. 246—Flow chart of AFOD 08 FROM FrIV1+IV4 PASTE

Description

PROCESS OF AFOD08 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), eluted with 2M Nacl TRIS-HCL buffer (PH8.50). Collect elute, called elute3.

10, to concentrate the solution to 7.5%? With 10 k ultra-filtration membrane, then dialysis with 10 volume of cold WFI

11, to carry out DV20 filtration

12, and adjust the PH value to 7.00.

13, to add albumin to concentration of 2.5%? as stabilizer.

14, to go to sterile filtration and filling.

FIGS. 247A&B—Flow chart of AFOD 09 FROM FrIV1+IV4 PASTE

Description

PROCESS OF AFOD09 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 nm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 nm, obtain the clear filtrate

9, to load the filtrate to column (resin DEAE FF), collect flowthrough.

10,to add alcohol to the flowthrough until the alcohol concentration is 40%.

11,to cool down the suspension to −5--7 C, and adjust the PH value to 5.80

12, to go to centrifugation, collect the paste, called paste 43

13, to dissolve the paste43 with TRIS-HCL buffer (PH8.50), dilution ratio is 1:9, temperature is 15-20 C

14, to perform filtration with depth filters such as 10 cp, 30 sp followed by 0.45 nm, obtain the clear filtrate

15, to concentrate the solution to 7.5%? With 10 k ultra-filtration membrane, collect the permeate

16, to concentrate the permeate to 3%? With 1-3 k ultra-filtration membrane, then dialysis with 10 volume of cold WFI

17, to carry out DV20 filtration

18,to adjust the PH value to 7.00.

19, to add albumin to concentration of 2.5%? as stabilizer.

20, to go to sterile filtration and filling.

FIGS. 248A&B—Flow chart of AFOD 10 FROM FrIV1+IV4 PASTE

Description

PROCESS OF AFOD 10 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), collect flowthrough.

10,to add alcohol to the flowthrough until the alcohol concentration is 40%.

11,to cool down the suspension to −5--7 C, and adjust the PH value to 5.80

12, to go to centrifugation, collect the paste, called paste 43

13, to dissolve the paste43 with TRIS-HCL buffer (PH8.50), dilution ratio is 1:9, temperature is 15-20 C

14, to perform filtration with depth filters such as 10 cp, 30 sp followed by 0.45 μm, obtain the clear filtrate

15, to concentrate the solution to 7.5%? With 10 k ultra-filtration membrane, then dialysis with 10 volume of cold WFI.

16, to carry out DV20 filtration

17, to adjust the PH value to 7.00.

18, to add albumin to concentration of 2.5%? as stabilizer.

19, to go to sterile filtration and filling.

FIGS. 249A&B—Flow chart of AFOD 11 FROM FrIV1+IV4 PASTE

Description

PROCESS OF AFOD11 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), collect flowthrough.

10,to add alcohol to the flowthrough until the alcohol concentration is 40%.

11,to cool down the suspension to −5˜-7 C, and adjust the PH value to 5.80

12, to go to centrifugation, collect supernatant

13, to perform filtration with depth filters such as 10 cp, 30 sp followed by 0.45 μm, obtain the clear filtrate

14,to load filtrate to column (resin DEAE sepharose FF),collect elute

15, to concentrate the elute to 2.5%? With 10 k ultra-filtration membrane, then dialysis with 10 volume of cold WFI

16, to carry out DV20 filtration

17, to concentrate to 5%? With 10 k ultra-filtration membrane,

18, and adjust the PH value to 7.00.

19, to add albumin to concentration of 2.5%? as stabilizer.

20, to go to sterile filtration and filling.

Description

FIGS. 250A&B—Flow chart of AFOD 12 FROM FrIV1+IV4 PASTE

PROCESS OF AFOD12 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect Apoa-I paste,

3, to dissolve the Apoa-I paste with TRIS-HCL buffer (PH8.50), dilution ratio is 1:9, temperature is 15-20 C

4, to go to centrifugation at temperature of 15-20 C, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?, then diluted with 1 volume of

cold WFI, add Nacl to 20 Mm

8, to perform filtration with depth filters such as 10 cp, 90 sp followed by 0.45 μm, obtain the clear filtrate

9, to load the filtrate to column (resin DEAE FF), collect flowthrough.

10,to add alcohol to the flowthrough until the alcohol concentration is 40%.

11,to cool down the suspension to −5--7 C, and adjust the PH value to 5.80

12, to go to centrifugation, collect supernatant

13, to perform filtration with depth filters such as 10 cp, 30 sp followed by 0.45 μm, obtain the clear filtrate

14,to load filtrate to column (resin DEAE sepharose FF),collect elute

15, to concentrate the elute to 2.5%? With 10 k ultra-filtration membrane, collect the permeate.

16, to concentrate the permeate to 2.5%? With 1-3K ultra-filtration membrane, then dialysis with 10 volume of cold WFI

17, to carry out DV20 filtration

18, to concentrate to 5%? With 1-3 k ultra-filtration membrane,

19, and adjust the PH value to 7.00.

20, to add albumin to concentration of 2.5%? as stabilizer.

21, to go to sterile filtration and filling.

FIGS. 251A&B—Flow chart of AFOD 13 FROM FrIV1+IV4 PASTE

Description

PROCESS OF AFOD13 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect filtrate,

3, to adjust PH value to 5.80?, dilution ratio is 1:9, temperature is 15-20 C

4, to go to centrifugation at temperature of 0-3 C?, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?,

8, to perform filtration with depth filters such as 10 cp, 90 sp followed by 0.45 μm, obtain the clear filtrate

9, to load the filtrate to column (resin DEAE FF), collect flow elute.

10, to perform filtration with depth filters such as 10 cp, 30 sp followed by 0.45 μm, obtain the clear filtrate

11,to load filtrate to column (resin DEAE sepharose FF),collect elute

12, to concentrate the elute to 5%? With 10 k ultra-filtration membrane, collect the permeate.

13, to concentrate the permeate to 2.5%? With 1-3K ultra-filtration membrane, then dialysis with 10 volume of cold WFI

14, to carry out DV20 filtration

15, and adjust the PH value to 7.00.

16, to add albumin to concentration of 2.5%? as stabilizer.

17, to go to sterile filtration and filling.

FIGS. 252A&B—Flow chart of AFOD 14 FROM FrIV1+IV4 PASTE

Description

PROCESS OF AFOD14 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect filtrate,

3, to adjust PH value to 5.80?,

4, to go to centrifugation at temperature of 0-3 C?, obtain the supernatant.

5, to perform filtration with depth filters such as 10 cp, 90 sp followed by 0.45 μm, obtain the clear filtrate.

6, to add tween80 to concentration of 1% and TNBP to 0.3%, then keep the temperature of the solution at 25 C for 6 hours.

7, to cool down the solution to temperature below 10 C and adjust PH value to about ?,

8, to perform filtration with depth filters such as 10 cp, 90 sp followed by 0.45 μm, obtain the clear filtrate

9, to load the filtrate to column (resin DEAE FF), collect elute.

10, to perform filtration with depth filters such as 10 cp, 30 sp followed by 0.45 μm, obtain the clear filtrate

11,to load filtrate to column (resin DEAE sepharose FF),collect elute

12, to concentrate the elute to 5%? With 10 k ultra-filtration membrane, then dialysis with 10 volume of cold WFI

13, to carry out DV20 filtration

14, to concentrate the solution to 20%? With 10 k ultra-filtration membrane,

15, and adjust the PH value to 7.00.

16, to add albumin to concentration of 2.5%? as stabilizer.

17, to go to sterile filtration and filling.

FIG. 253A—Flow chart of AFOD 15 FROM FrIV1+IV4 PASTE

Description

PROCESS OF AFOD15 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect paste, called paste42.

3, to dissolve the paste, dilution ratio is 1:9?, temperature is 15-20 C?

4, to perform filtration with depth filters such as 10 cp, 90 sp followed by 0.45 μm, obtain the clear filtrate.

5, to concentrate the filtrate to 3%? With 10 k ultra-filtration membrane, collect the permeate.

6, to concentrate the permeate to 2.5%? With 1-3K ultra-filtration membrane, then dialysis with 10 volume of cold WFI

7, to carry out DV20 filtration

8, to adjust the PH value to 7.00.

9, to add albumin to concentration of 2.5%? as stabilizer.

10, to go to sterile filtration and filling.

FIG. 254—Flow chart of AFOD 16 FROM FrIV1+IV4 PASTE

Description

PROCESS OF AFOD16 FROM FrIV1+IV4 PASTE

1, Firstly to dissolve the Fr.IV1+IV4 paste with cold WFI, dilution ratio is 1:9,then add sodium acetate to concentration of 20 mM and adjust PH value of the suspension to about 6.00, to agitate at sufficient rate until fully dissolved.

2, to cool down the suspension to temperature of 0 C, then perform press filtration with filters such as endures, s100 and

0.45 μm, etc. collect paste, called paste42.

3, to dissolve the paste, dilution ratio is 1:9?, temperature is 15-20 C?

4, to perform filtration with depth filters such as 10 cp, 90 sp followed by 0.45 μm, obtain the clear filtrate.

5, to concentrate the filtrate to 3%? With 10 k ultra-filtration membrane, then dialysis with 10 volume of cold WFI

6, to carry out DV20 filtration

7,to adjust the PH value to 7.00.

8, to add albumin to concentration of 2.5%? as stabilizer.

9, to go to sterile filtration and filling.

FIG. 255—Cryopaste and FVIII

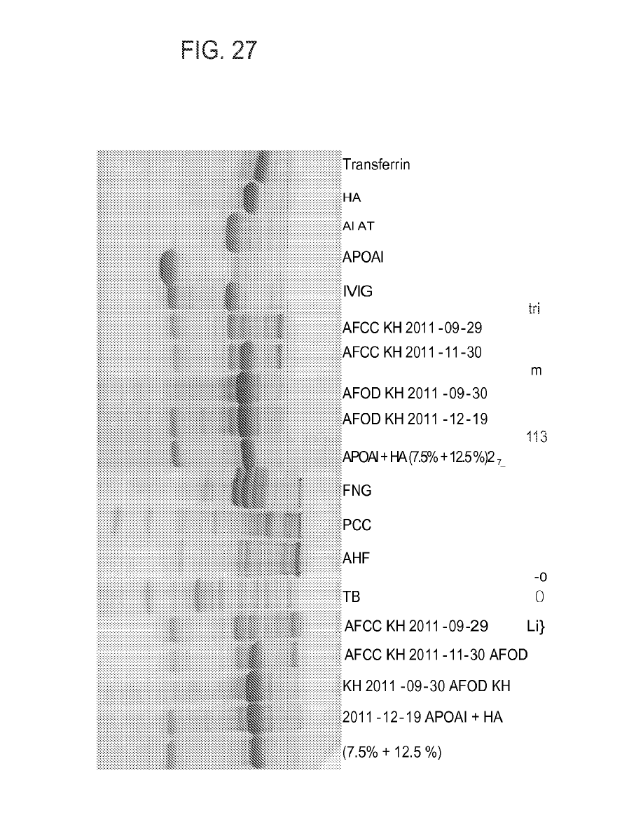

See FIGS. 256-265 and 27.

1. The process of obtaining 30% or higher of a protein selected from the group consisting of Human Albumin protein, Human Albumin uncharacterized protein, HPR 31 kDa protein, AIBG isoform 1 of Alpha-1b-glycoprotein protein, HPR haptoglobin protein, ACTC1 Actin protein, Alpha cardiac muscle 1, KH51 protein, Immunoglobulin proteins from fraction II, 120/E19 IGHV4-31 protein, IGHG1 44 kDa protein, 191/H18 IGHV4-31 protein, IGHG1 32 kDa, IGHV4-31 protein, IGHG1 putative uncharacterized protein, KH 33 protein, KH 34 protein, KH 35 protein, KH 36 protein, KH37 protein, Hepatitis B immunoglobulin protein from fraction II, TF protein sequence#197/H24 protein, TF serotransferrin protein, Immunoglobulin protein from fraction III, 193/H20 TF serotransferrin protein, 194/H21 APOH beta2-glycoprotein 1 protein, 195/H22 cDNA FLJ5165 protein, beta-2-glycoprotein protein, 196/H23 FCN3 isoform 1 of Ficolin-3 protein, KH 3 protein, KH 4 protein, KH 5 protein, KH 6 protein, KH 7 protein, KH 8 protein, KH 9 protein, KH 10 protein, KH 41 protein, KH 42 protein, KH 43 protein, in KH healthy cells in which the RNA synthesizes good proteins: 1—Send signals to the DAMAGED, SICK, AND BAD CELLS that triggers that synthesis of good proteins that transform these cells to become GOOD healthy cells; 2—Send signals to the other currently undamaged cells to synthesis of good proteins to protect them from being DAMAGED, INFECTED and PRONE to DNA and other cellular alterations; 3—Send signals to the body to produce new cells that are healthy and forbid them from being affected by intra- and extracellular damaging signals in order to cure diseases, viruses infections, bacteria infections, auto immune disease, neurological disorder, all type of solid and blood cancer, coagulation, diabetic, inhibitor, immune deficiency, muscle and nerve repair and restoration.

2. The process of claim 1, wherein the protein is Human Albumin uncharacterized protein.

3. The process of claim 1, wherein the protein is HPR 31 kDa protein.

4. The process of claim 1, wherein the protein is AIBG isoform 1 of Alpha-1b-glycoprotein protein.

5. The process of claim 1, wherein the protein is HPR haptoglobin protein.

6. The process of claim 1, wherein the protein is ACTC1 Actin protein.

7. The process of claim 1, wherein the protein is Alpha cardiac muscle 1 protein.

8. The process of claim 1, wherein the protein is KH51 protein.

9. The process of claim 1, wherein the protein is any combination of any of the following proteins found in Human Albumin: Human Albumin uncharacterized, HPR 31 kDa, AIBG isoform 1 of Alpha-1b-glycoprotein, HPR haptoglobin, ACTC1 Actin, Alpha cardiac muscle 1 and KH51 protein.

10. The process of claim 1, wherein the protein is HPR 31 kDa, ACTC1 Actin, Alpha cardiac muscle 1 and KH51 protein can only be found in Human Albumin with trademark AlbuRAAS®.

11. The process of claim 1, wherein the protein is an Immunoglobulin protein from fraction II.

12. The process of claim 1, wherein the protein is 120/E19 IGHV4-31 protein.

13. The process of claim 1, wherein the protein is IGHG1 44 kDa protein.

14. The process of claim 1, wherein the protein is 191/H18 IGHV4-31 protein.

15. The process of claim 1, wherein the protein is IGHG1 32 kDa protein.

16. The process of claim 1, wherein the protein is IGHV4-31 protein.

17. The process of claim 1, wherein the protein is IGHG1 putative uncharacterized protein DKFZp686G11190 protein

18. The process of claim 1, wherein the protein is KH33 protein.

19. The process of claim 1, wherein the protein is KH34 protein.

20. The process of claim 1, wherein the protein is KH35 protein.
